(19) United States
(12) Patent Application Publication
(10) Pub. No.: US 2024/0008360 A1
(43) Pub. Date: Jan. 4, 2024

(54) HETEROCYCLIC COMPOUND, ORGANIC LIGHT-EMITTING DEVICE COMPRISING SAME, MANUFACTURING METHOD THEREFOR, AND COMPOSITION FOR ORGANIC LAYER

(71) Applicant: LT MATERIALS CO., LTD., Yongin-si, Gyeonggi-do (KR)

(72) Inventors: Nam Jin LEE, Yongin-si (KR); GWANG IL DONG, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/276,084

(22) PCT Filed: Mar. 15, 2022
(86) PCT No.: PCT/KR2022/003589

§ 371 Date: Aug. 7, 2023

(30) Foreign Application Priority Data
Mar. 31, 2021 (KR) 10-2021-0041901

(51) Int. Cl. H10K 85/60; C07D 498/06

(52) U.S. Cl. CPC H10K 85/636; C07D 498/06; H10K 85/633; H10K 50/11

(57) ABSTRACT

The present specification relates to a heterocyclic compound represented by Chemical Formula 1, an organic light emitting device comprising the same, a method for manufacturing the same, and a composition for an organic material layer.

### TECHNICAL FIELD

This application claims priority to and the benefits of Korean Patent Application No. 10-2021-0041901, filed with the Korean Intellectual Property Office on Mar. 31, 2021, the entire contents of which are incorporated herein by reference.

The present disclosure relates to a heterocyclic compound, an organic light emitting device comprising the same and a method for manufacturing the same.

### BACKGROUND ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### Prior Art Documents

### Patent Documents

U.S. Pat. No. 4,356,429

### DISCLOSURE

### Technical Problem

An object of the present disclosure is to provide a heterocyclic compound, an organic light emitting device comprising the same and a method for manufacturing the same.

### Technical Solution

In order to achieve the above object, the present disclosure provides a heterocyclic compound represented by the following Chemical Formula 1.

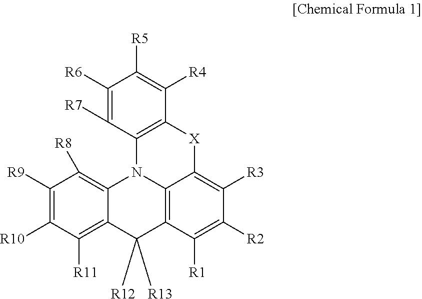

In Chemical Formula 1,
X is O or S,
R1 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R1 to R13 is a group represented by the following Chemical Formula 2,

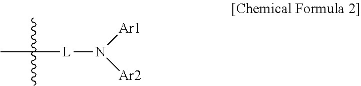

in Chemical Formula 2,
Ar1 and Ar2 are the same as or different from each other, and each independently a substituted or unsubstituted C6 to C60 aryl group; or a substituted or unsubstituted C2 to C60 heteroaryl group, and
L is a direct bond; a substituted or unsubstituted C6 to C60 arylene group; or a substituted or unsubstituted C2 to C60 heteroarylene group.

In addition, one embodiment of the present disclosure provides an organic light emitting device comprising:
a first electrode; a second electrode provided to face the first electrode; and one or more organic material layers provided between the first electrode and the second electrode, wherein one or more layers of the organic material layers comprise the heterocyclic compound represented by Chemical Formula 1.

In addition, one embodiment of the present disclosure provides an organic light emitting device, wherein the organic material layer further comprises a heterocyclic compound represented by the following Chemical Formula 3 or Chemical Formula 4.

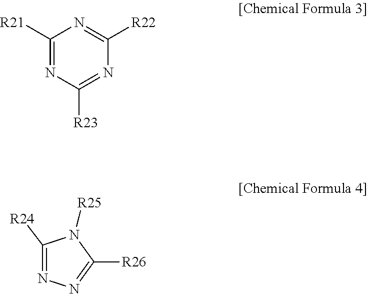

In Chemical Formula 3 and Chemical Formula 4,
R21 to R26 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group.

In addition, one embodiment of the present disclosure provides a composition for an organic material layer of an organic light emitting device, the composition including the heterocyclic compound represented by Chemical Formula 1 and the heterocyclic compound represented by Chemical Formula 3 or Chemical Formula 4.

In addition, one embodiment of the present disclosure provides a method for manufacturing an organic light emitting device, the method comprising the steps of:
preparing a substrate; forming a first electrode on the substrate; forming one or more organic material layers on the first electrode; and forming a second electrode on the one or more organic material layers, wherein the forming of one or more organic material layers includes forming the one or more organic material layers using the composition for an organic material layer of an organic light emitting device.

### Advantageous Effects

The compound described in the present specification can be used as a material of an organic material layer of an organic light emitting device. The compound is capable of performing a role of a hole injection layer material, an electron blocking layer material, a hole transfer layer material, a light emitting layer material, an electron transfer layer material, a hole blocking layer material, an electron injection layer material and the like in an organic light emitting device. Particularly, the compound can be used as a hole transfer layer material, an electron blocking layer material or a light emitting layer material of an organic light emitting device.

Specifically, the compound can be used either alone as a light emitting material, and can be used as a host material or a dopant material of a light emitting layer. In addition, as the host material of a light emitting layer, the compound represented by Chemical Formula 1 may be used either alone, or a plurality of host materials may be mixed and used. Using the compound represented by Chemical Formula 1 in an organic material layer is capable of lowering a driving voltage of an organic light emitting device, enhancing light emission efficiency, and enhancing lifetime properties.

Particularly, the heterocyclic compound represented by Chemical Formula 1 of the present disclosure enhances electron stability and mobility by the LUMO being delocalized, which is effective in enhancing a lifetime of an organic electroluminescent device.

In addition, the heterocyclic compound represented by Chemical Formula 1 of the present disclosure prevents reversed energy transfer from a dopant to a host by having a high triplet energy level (T₁ level), and is effective in well-conserving triplet excitons in a light emitting layer.

In addition, the heterocyclic compound represented by Chemical Formula 1 of the present disclosure facilitates charge transfer in the molecule, and well-conserves excitons by reducing an energy gap between the singlet energy level (Si) and the triplet energy level (T₁).

### DESCRIPTION OF DRAWINGS

FIG. 1 to FIG. 3 are diagrams each schematically illustrating a lamination structure of an organic light emitting device according to one embodiment of the present disclosure.

### MODE FOR DISCLOSURE

Hereinafter, the present application will be described in detail.

In the present specification, a term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent linking two or more substituents selected from among the substituents illustrated above or being unsubstituted.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, an n-propyl group, an isopropyl group, an n-butyl group, an isobutyl group, a tert-butyl group, a sec-butyl group, a 1-methyl-butyl group, a 1-ethyl-butyl group, an n-pentyl group, an isopentyl group, a neopentyl group, a tert-pentyl group, an n-hexyl group, a 1-methylpentyl group, a 2-methylpentyl group, a 4-methyl-2-pentyl group, a 3,3-dimethylbutyl group, a 2-ethylbutyl group, an n-heptyl group, a 1-methylhexyl group, a cyclopentylmethyl group, a cyclohexylmethyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be used. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR104R105R106. R104 to R106 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

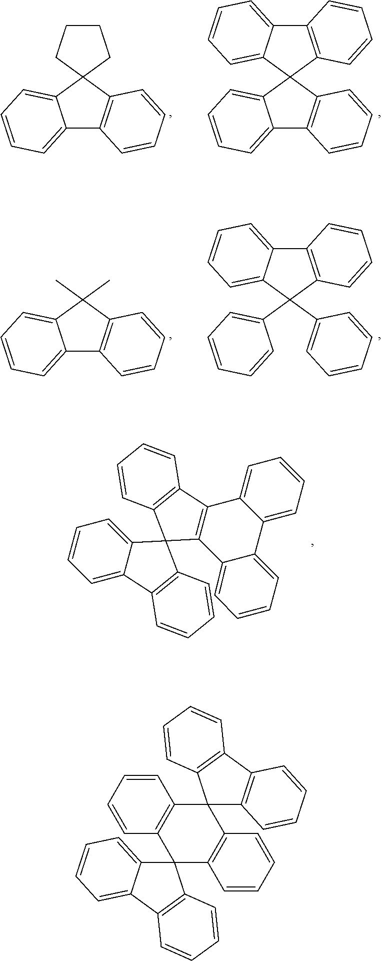

and the like may be included, however, the structure is not limited thereto.

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be mixed in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

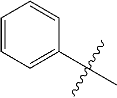 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

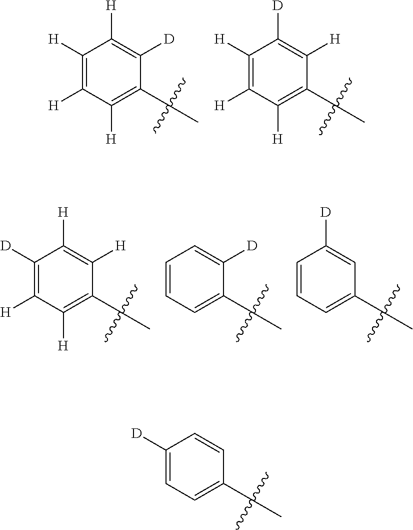 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the deuterium content may be from 0% to 100% and more preferably from 30% to 100% in the heterocyclic compound represented by Chemical Formula 1.

In the present disclosure, the C6 to C60 aromatic hydrocarbon ring means a compound including an aromatic ring formed with C6 to C60 carbons and hydrogens. Examples thereof may include benzene, biphenyl, triphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art satisfying the above-mentioned number of carbon atoms.

One embodiment of the present disclosure provides a heterocyclic compound represented by the following Chemical Formula 1.

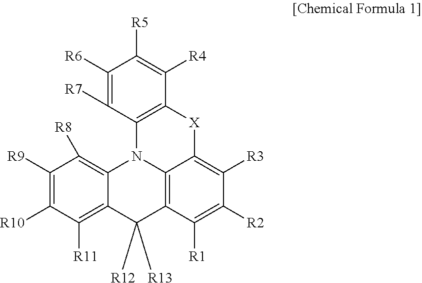

In Chemical Formula 1,
X is O or S,
R1 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
at least one of R1 to R13 is a group represented by the following Chemical Formula 2,

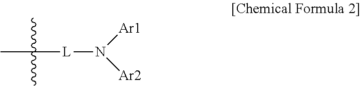

in Chemical Formula 2,
Ar1 and Ar2 are the same as or different from each other, and each independently a substituted or unsubstituted C6 to C60 aryl group; or a substituted or unsubstituted C2 to C60 heteroaryl group, and
L is a direct bond; a substituted or unsubstituted C6 to C60 arylene group; or a substituted or unsubstituted C2 to C60 heteroarylene group.

In one embodiment of the present disclosure, X may be O.

In another embodiment of the present disclosure, X may be S.

In one embodiment of the present disclosure, R1 to R11 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or the group represented by Chemical Formula 2.

In another embodiment of the present disclosure, R1 to R11 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or the group represented by Chemical Formula 2.

In another embodiment of the present disclosure, R1 to R11 are the same as or different from each other, and may be each independently hydrogen; deuterium; a substituted or unsubstituted C1 to C20 alkyl group; a substituted or unsubstituted C6 to C20 aryl group; a substituted or unsubstituted C2 to C20 heteroaryl group; or the group represented by Chemical Formula 2.

In another embodiment of the present disclosure, R1 to R11 are the same as or different from each other, and may be each independently hydrogen; deuterium; a substituted or unsubstituted methyl group, ethyl group or propyl group; a substituted or unsubstituted phenyl group, naphthyl group, phenanthrenyl group or isochrysenyl group; a substituted or unsubstituted dibenzofuranyl group, dibenzothiophenyl group or carbazolyl group; or the group represented by Chemical Formula 2.

In another embodiment of the present disclosure, R1 to R11 are the same as or different from each other, and may be each independently hydrogen; deuterium; a substituted or unsubstituted methyl group, ethyl group or propyl group; a substituted or unsubstituted phenyl group, naphthyl group, phenanthrenyl group or isochrysenyl group; or the group represented by Chemical Formula 2.

In one embodiment of the present disclosure, at least one of R1 to R11 may be the group represented by Chemical Formula 2.

In another embodiment of the present disclosure, one or two of R1 to R11 may be the group represented by Chemical Formula 2.

In one embodiment of the present disclosure, R12 and R13 are the same as or different from each other, and may be each independently a substituted or unsubstituted C1 to C20 alkyl group.

In another embodiment of the present disclosure, R12 and R13 are the same as or different from each other, and may be each independently a substituted or unsubstituted C1 to C10 alkyl group.

In another embodiment of the present disclosure, R12 and R13 are the same as or different from each other, and may be each independently a substituted or unsubstituted methyl group, ethyl group or propyl group.

In another embodiment of the present disclosure, R12 and R13 may all be a methyl group.

In one embodiment of the present disclosure, Ar1 and Ar2 are the same as or different from each other, and may be each independently a substituted or unsubstituted C6 to C30 aryl group; or a substituted or unsubstituted C2 to C30 heteroaryl group.

In another embodiment of the present disclosure, Ar1 and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted phenyl group, naphthyl group, fluorenyl group, phenanthrenyl group, isochrysenyl group or spirodifluorenyl group; or a substituted or unsubstituted dibenzofuranyl group or dibenzothiophenyl group.

In one embodiment of the present disclosure, L may be a direct bond; a substituted or unsubstituted C6 to C30 arylene group; or a substituted or unsubstituted C2 to C30 heteroarylene group.

In another embodiment of the present disclosure, L may be a direct bond; a substituted or unsubstituted C6 to C20 arylene group; or a substituted or unsubstituted C2 to C20 heteroarylene group.

In another embodiment of the present disclosure, L may be a direct bond; a substituted or unsubstituted phenylene group or biphenylene group.

Specific examples of L are shown below, however, L is not limited to these examples.

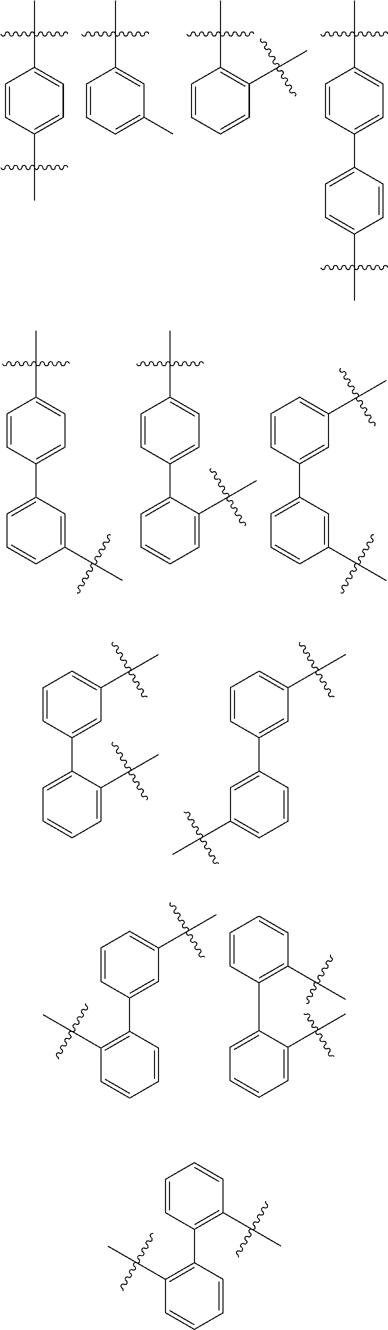

In one embodiment of the present disclosure, Chemical Formula 1 may be a heterocyclic compound represented by any one of the following Chemical Formula 1-1 to Chemical Formula 1-3.

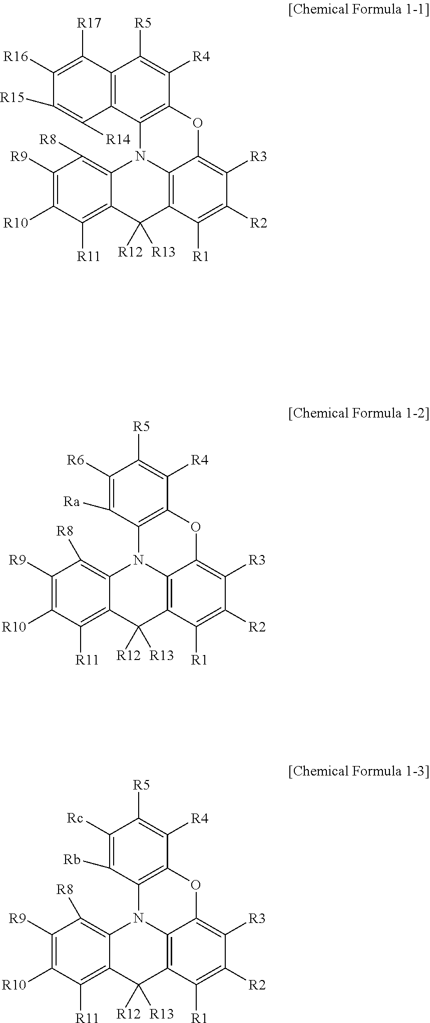

In Chemical Formula 1-1 to Chemical Formula 1-3,
R14 to R17 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and the group represented by Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
Ra is a substituted or unsubstituted C1 to C60 alkyl group,
Rb and Rc are the same as or different from each other, and each independently hydrogen; deuterium; a substituted or unsubstituted C6 to C60 aryl group; a substituted or unsubstituted C2 to C60 heteroaryl group; or the group represented by Chemical Formula 2, and
R1 to R6 and R8 to R13 have the same definitions as in Chemical Formula 1.

In one embodiment of the present disclosure, R14 to R17 are the same as or different from each other, and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or the group represented by Chemical Formula 2.

In another embodiment of the present disclosure, R14 to R17 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or the group represented by Chemical Formula 2.

In another embodiment of the present disclosure, R14 to R17 are the same as or different from each other, and may be each independently hydrogen; deuterium; a substituted or unsubstituted C6 to C20 aryl group; or a substituted or unsubstituted C2 to C20 heteroaryl group.

In another embodiment of the present disclosure, R14 to R17 are the same as or different from each other, and may be each independently hydrogen; or deuterium.

In one embodiment of the present disclosure, Ra may be a substituted or unsubstituted C1 to C60 alkyl group.

In another embodiment of the present disclosure, Ra may be a substituted or unsubstituted C1 to C30 alkyl group.

In another embodiment of the present disclosure, Ra may be a substituted or unsubstituted C1 to C10 alkyl group.

In another embodiment of the present disclosure, Ra may be a substituted or unsubstituted methyl group, ethyl group or propyl group.

In another embodiment of the present disclosure, Ra may be a substituted or unsubstituted methyl group.

In one embodiment of the present disclosure, Rb and Rc are the same as or different from each other, and may be each independently hydrogen; deuterium; a substituted or unsubstituted C6 to C30 aryl group; a substituted or unsubstituted C2 to C30 heteroaryl group; or the group represented by Chemical Formula 2.

In another embodiment of the present disclosure, Rb and Rc are the same as or different from each other, and may be each independently hydrogen; deuterium; a substituted or unsubstituted C6 to C20 aryl group; a substituted or unsubstituted C2 to C20 heteroaryl group; or the group represented by Chemical Formula 2.

In another embodiment of the present disclosure, Rb and Rc are the same as or different from each other, and may be each independently hydrogen; deuterium; a substituted or unsubstituted C6 to C20 aryl group; or the group represented by Chemical Formula 2.

In another embodiment of the present disclosure, Rb and Rc are the same as or different from each other, and may be each independently hydrogen; deuterium; a substituted or unsubstituted phenyl group, naphthyl group or phenanthrenyl group; or the group represented by Chemical Formula 2.

In another embodiment of the present disclosure, when Rb is hydrogen; or deuterium, Rc may be hydrogen; deuterium; a substituted or unsubstituted C6 to C20 aryl group; or the group represented by Chemical Formula 2.

In another embodiment of the present disclosure, when Rc is hydrogen; or deuterium, Rb may be hydrogen; deuterium; a substituted or unsubstituted C6 to C20 aryl group; or the group represented by Chemical Formula 2.

In another embodiment of the present disclosure, when Rb is a substituted or unsubstituted C6 to C30 aryl group, Rc may be hydrogen; or deuterium.

In another embodiment of the present disclosure, when Rb is the group represented by Chemical Formula 2, Rc may be hydrogen; or deuterium.

In another embodiment of the present disclosure, when Rc is a substituted or unsubstituted C6 to C30 aryl group, Rb may be hydrogen; or deuterium.

In another embodiment of the present disclosure, when Rc is the group represented by Chemical Formula 2, Rb may be hydrogen; or deuterium.

In one embodiment of the present disclosure, at least one of R1 to R3 and R9 to R11 of Chemical Formula 1-1 may be the group represented by Chemical Formula 2.

In another embodiment of the present disclosure, when at least one of R1 to R3 in Chemical Formula 1-1 is the group represented by Chemical Formula 2, R9 to R11 are the same as or different from each other and may be each independently hydrogen; deuterium; or a substituted or unsubstituted C6 to C60 aryl group, and
when at least one of R9 to R11 is the group represented by Chemical Formula 2, R1 to R3 are the same as or different from each other and may be each independently hydrogen; deuterium; or a substituted or unsubstituted C6 to C60 aryl group.

In another embodiment of the present disclosure, when at least one of R1 to R3 in Chemical Formula 1-1 is the group represented by Chemical Formula 2, R9 to R11 are the same as or different from each other and may be each independently hydrogen; deuterium; or a substituted or unsubstituted C6 to C30 aryl group, and
when at least one of R9 to R11 is the group represented by Chemical Formula 2, R1 to R3 are the same as or different from each other and may be each independently hydrogen; deuterium; or a substituted or unsubstituted C6 to C30 aryl group.

In another embodiment of the present disclosure, when at least one of R1 to R3 in Chemical Formula 1-1 is the group represented by Chemical Formula 2, R9 to R11 are the same as or different from each other and may be each independently hydrogen; deuterium; or a substituted or unsubstituted C6 to C20 aryl group, and
when at least one of R9 to R11 is the group represented by Chemical Formula 2, R1 to R3 are the same as or different from each other and may be each independently hydrogen; deuterium; or a substituted or unsubstituted C6 to C20 aryl group.

In another embodiment of the present disclosure, when at least one of R1 to R3 in Chemical Formula 1-1 is the group represented by Chemical Formula 2, R9 to R11 are the same as or different from each other and may be each independently hydrogen; deuterium; or a substituted or unsubstituted phenyl group, naphthyl group, phenanthrenyl group or isochrysenyl group, and
when at least one of R9 to R11 is the group represented by Chemical Formula 2, R1 to R3 are the same as or different from each other and may be each independently hydrogen; deuterium; or a substituted or unsubstituted phenyl group, naphthyl group, phenanthrenyl group or isochrysenyl group.

In another embodiment of the present disclosure, when at least one of R1 to R3 in Chemical Formula 1-1 is the group represented by Chemical Formula 2, at least one of R9 to R11 may be a substituted or unsubstituted C6 to C20 aryl group, and
when at least one of R9 to R11 is the group represented by Chemical Formula 2, at least one of R1 to R3 may be a substituted or unsubstituted C6 to C20 aryl group.

In one embodiment of the present disclosure, at least one of R1 to R6 and R9 to R11 in Chemical Formula 1-2 may be the group represented by Chemical Formula 2.

In another embodiment of the present disclosure, when at least one of R1 to R3 in Chemical Formula 1-2 is the group represented by Chemical Formula 2, R4 to R6 and R9 to R11 are the same as or different from each other and may be each independently hydrogen; deuterium; or a substituted or unsubstituted C6 to C60 aryl group,
when at least one of R4 to R6 is the group represented by Chemical Formula 2, R1 to R3 and R9 to R11 are the same as or different from each other and may be each independently hydrogen; deuterium; or a substituted or unsubstituted C6 to C60 aryl group, and
when at least one of R9 to R11 is the group represented by Chemical Formula 2, R1 to R6 are the same as or different from each other and may be each independently hydrogen; deuterium; or a substituted or unsubstituted C6 to C60 aryl group.

In another embodiment of the present disclosure, when at least one of R1 to R3 in Chemical Formula 1-2 is the group represented by Chemical Formula 2, R4 to R6 and R9 to R11 are the same as or different from each other and may be each independently hydrogen; deuterium; or a substituted or unsubstituted C6 to C30 aryl group,
when at least one of R4 to R6 is the group represented by Chemical Formula 2, R1 to R3 and R9 to R11 are the same as or different from each other and may be each independently hydrogen; deuterium; or a substituted or unsubstituted C6 to C30 aryl group, and
when at least one of R9 to R11 is the group represented by Chemical Formula 2, R1 to R6 are the same as or different from each other and may be each independently hydrogen; deuterium; or a substituted or unsubstituted C6 to C30 aryl group.

In another embodiment of the present disclosure, when at least one of R1 to R3 in Chemical Formula 1-2 is the group represented by Chemical Formula 2, R4 to R6 and R9 to R11 are the same as or different from each other and may be each independently hydrogen; deuterium; or a substituted or unsubstituted C6 to C20 aryl group,
when at least one of R4 to R6 is the group represented by Chemical Formula 2, R1 to R3 and R9 to R11 are the same as or different from each other and may be each independently hydrogen; deuterium; or a substituted or unsubstituted C6 to C20 aryl group, and
when at least one of R9 to R11 is the group represented by Chemical Formula 2, R1 to R6 are the same as or different from each other and may be each independently hydrogen; deuterium; or a substituted or unsubstituted C6 to C20 aryl group.

In another embodiment of the present disclosure, when at least one of R1 to R3 in Chemical Formula 1-2 is the group represented by Chemical Formula 2, R4 to R6 and R9 to R11 are the same as or different from each other and may be each independently hydrogen; deuterium; or a substituted or unsubstituted phenyl group, naphthyl group, phenanthrenyl group or isochrysenyl group,
when at least one of R4 to R6 is the group represented by Chemical Formula 2, R1 to R3 and R9 to R11 are the same as or different from each other and may be each independently hydrogen; deuterium; or a substituted or unsubstituted phenyl group, naphthyl group, phenanthrenyl group or isochrysenyl group, and
when at least one of R9 to R11 is the group represented by Chemical Formula 2, R1 to R6 are the same as or different from each other and may be each independently hydrogen; deuterium; or a substituted or unsubstituted phenyl group, naphthyl group, phenanthrenyl group or isochrysenyl group.

In another embodiment of the present disclosure, when at least one of R1 to R3 in Chemical Formula 1-2 is the group represented by Chemical Formula 2, at least one of R4 to R6 and R9 to R11 may be a substituted or unsubstituted C6 to C20 aryl group,
when at least one of R4 to R6 is the group represented by Chemical Formula 2, at least one of R1 to R3 and R9 to R11 is a substituted or unsubstituted C6 to C20 aryl group, and
when at least one of R9 to R11 is the group represented by Chemical Formula 2, at last one of R1 to R6 may be a substituted or unsubstituted C6 to C20 aryl group.

In one embodiment of the present disclosure, at least one of R1 to R7 and R9 to R11 in Chemical Formula 1-3 may be the group represented by Chemical Formula 2.

In another embodiment of the present disclosure, when at least one of R1 to R3 in Chemical Formula 1-3 is the group represented by Chemical Formula 2, R4 to R7 and R9 to R11 are the same as or different from each other and may be each independently hydrogen; deuterium; or a substituted or unsubstituted C6 to C60 aryl group,
when at least one of R4 to R7 is the group represented by Chemical Formula 2, R1 to R3 and R9 to R11 are the same as or different from each other and may be each independently hydrogen; deuterium; or a substituted or unsubstituted C6 to C60 aryl group, and
when at least one of R9 to R11 is the group represented by Chemical Formula 2, R1 to R7 are the same as or different from each other and may be each independently hydrogen; deuterium; or a substituted or unsubstituted C6 to C60 aryl group.

In another embodiment of the present disclosure, when at least one of R1 to R3 in Chemical Formula 1-3 is the group represented by Chemical Formula 2, R4 to R7 and R9 to R11 are the same as or different from each other and may be each independently hydrogen; deuterium; or a substituted or unsubstituted C6 to C30 aryl group,
when at least one of R4 to R7 is the group represented by Chemical Formula 2, R1 to R3 and R9 to R11 are the same as or different from each other and may be each independently hydrogen; deuterium; or a substituted or unsubstituted C6 to C30 aryl group, and
when at least one of R9 to R11 is the group represented by Chemical Formula 2, R1 to R7 are the same as or different from each other and may be each independently hydrogen; deuterium; or a substituted or unsubstituted C6 to C30 aryl group.

In another embodiment of the present disclosure, when at least one of R1 to R3 in Chemical Formula 1-3 is the group represented by Chemical Formula 2, R4 to R7 and R9 to R11 are the same as or different from each other and may be each independently hydrogen; deuterium; or a substituted or unsubstituted C6 to C20 aryl group,
when at least one of R4 to R7 is the group represented by Chemical Formula 2, R1 to R3 and R9 to R11 are the same as or different from each other and may be each independently hydrogen; deuterium; or a substituted or unsubstituted C6 to C20 aryl group, and
when at least one of R9 to R11 is the group represented by Chemical Formula 2, R1 to R7 are the same as or different from each other and may be each independently hydrogen; deuterium; or a substituted or unsubstituted C6 to C20 aryl group.

In another embodiment of the present disclosure, when at least one of R1 to R3 in Chemical Formula 1-3 is the group represented by Chemical Formula 2, R4 to R7 and R9 to R11 are the same as or different from each other and may be each independently hydrogen; deuterium; or a substituted or unsubstituted phenyl group, naphthyl group, phenanthrenyl group or isochrysenyl group,
when at least one of R4 to R7 is the group represented by Chemical Formula 2, R1 to R3 and R9 to R11 are the same as or different from each other and may be each independently hydrogen; deuterium; or a substituted or unsubstituted phenyl group, naphthyl group, phenanthrenyl group or isochrysenyl group, and
when at least one of R9 to R11 is the group represented by Chemical Formula 2, R1 to R7 are the same as or different from each other and may be each independently hydrogen; deuterium; or a substituted or unsubstituted phenyl group, naphthyl group, phenanthrenyl group or isochrysenyl group.

In another embodiment of the present disclosure, when at least one of R1 to R3 in Chemical Formula 1-3 is the group represented by Chemical Formula 2, at least one of R4 to R7 and R9 to R11 may be a substituted or unsubstituted C6 to C20 aryl group,
when at least one of R4 to R7 is the group represented by Chemical Formula 2, at least one of R1 to R3 and R9 to R11 may be a substituted or unsubstituted C6 to C20 aryl group, and
when at least one of R9 to R11 is the group represented by Chemical Formula 2, at least one of R1 to R7 may be a substituted or unsubstituted C6 to C20 aryl group.

In one embodiment of the present disclosure, the deuterium content in Chemical Formula 1 may be 0% or greater, 10% or greater, 20% or greater, 30% or greater, 40% or greater or 50% or greater, and may be 100% or less, 90% or less, 80% or less, 70% or less or 60% or less based on the total number of hydrogen atoms and deuterium atoms.

In another embodiment of the present disclosure, the deuterium content in Chemical Formula 1 may be from 30% to 100% based on the total number of hydrogen atoms and deuterium atoms.

In another embodiment of the present disclosure, the deuterium content in Chemical Formula 1 may be from 30% to 80% based on the total number of hydrogen atoms and deuterium atoms.

In another embodiment of the present disclosure, the deuterium content in Chemical Formula 1 may be from 50% to 60% based on the total number of hydrogen atoms and deuterium atoms.

In one embodiment of the present disclosure, Chemical Formula 1 may be a heterocyclic compound represented by any one of the following compounds.

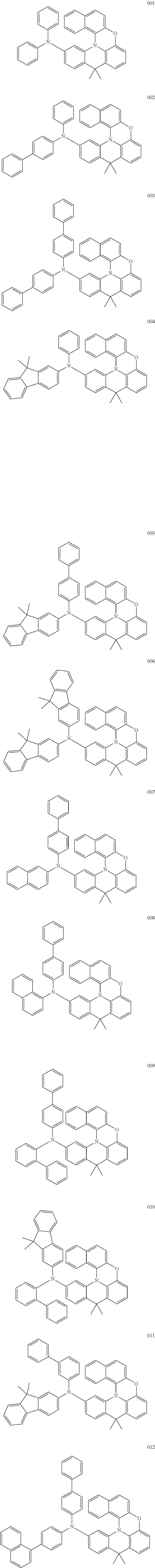

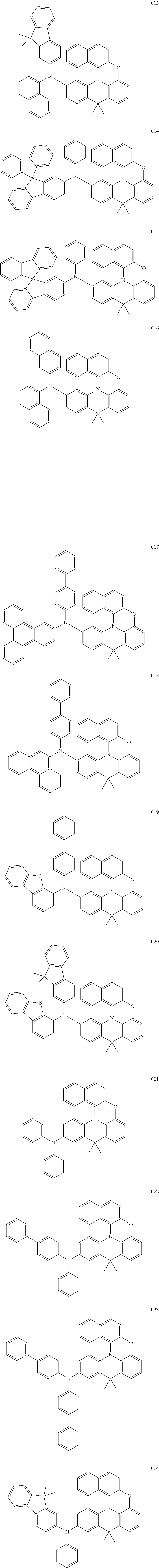

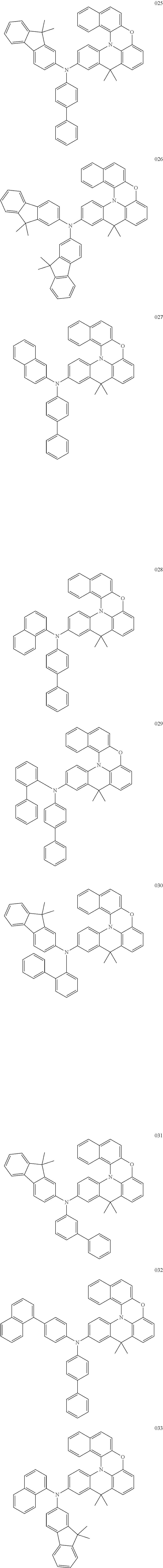

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

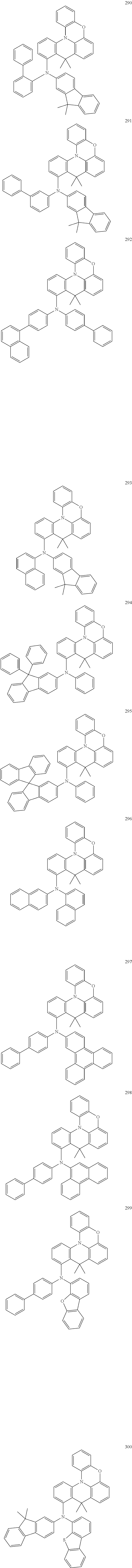

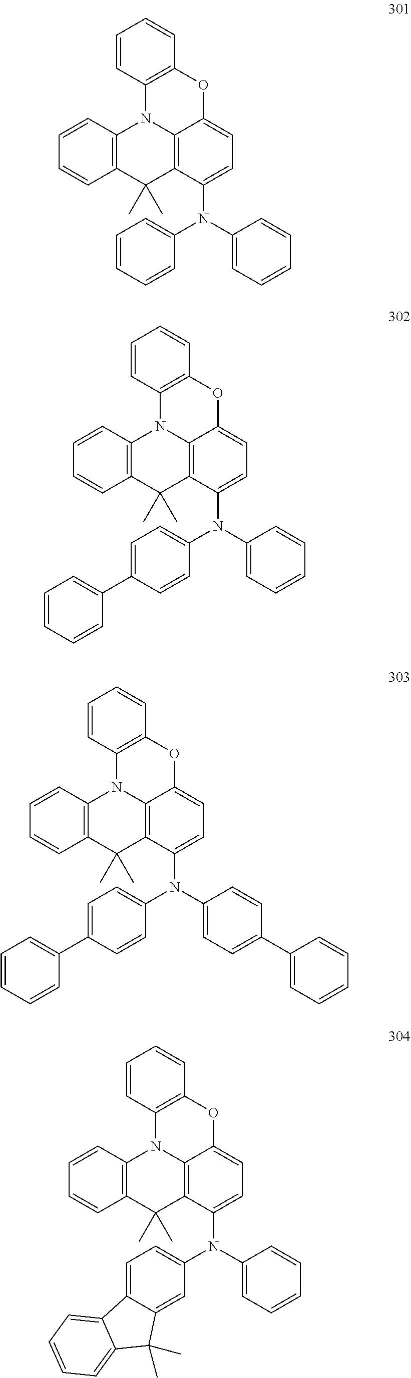

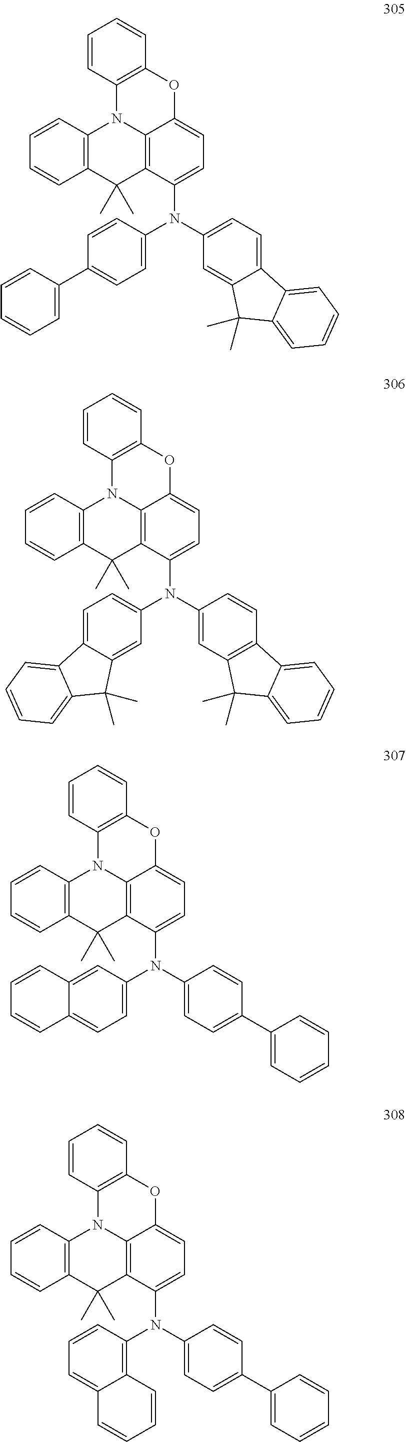

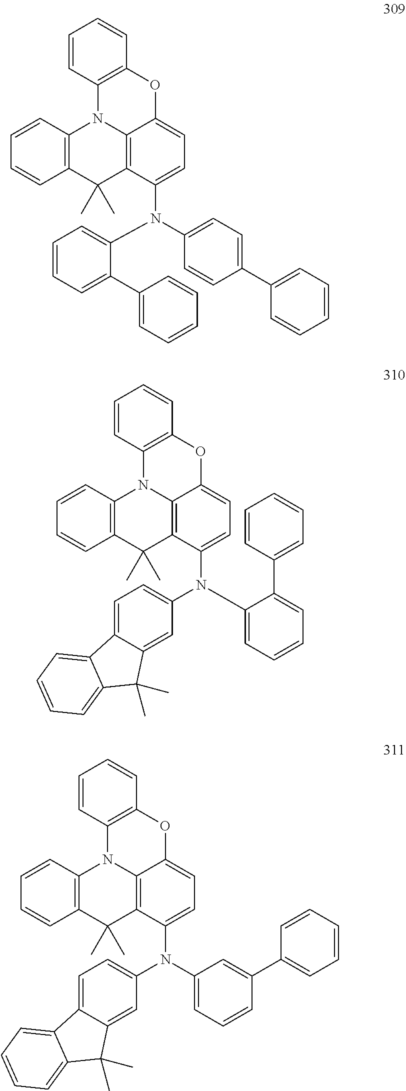 [SMILES: CC1(C)c2ccccc2N2c3ccccc3Oc3ccc(N(c4ccc(-c5ccccc5)cc4)c4ccccc4-c4ccccc4)c1c32 ; CC1(C)c2ccccc2-c2ccc(N(c3ccccc3-c3ccccc3)c3ccc4c5c3C(C)(C)c3ccccc3N5c3ccccc3O4)cc21 ; CC1(C)c2ccccc2-c2ccc(N(c3cccc(-c4ccccc4)c3)c3ccc4c5c3C(C)(C)c3ccccc3N5c3ccccc3O4)cc21]

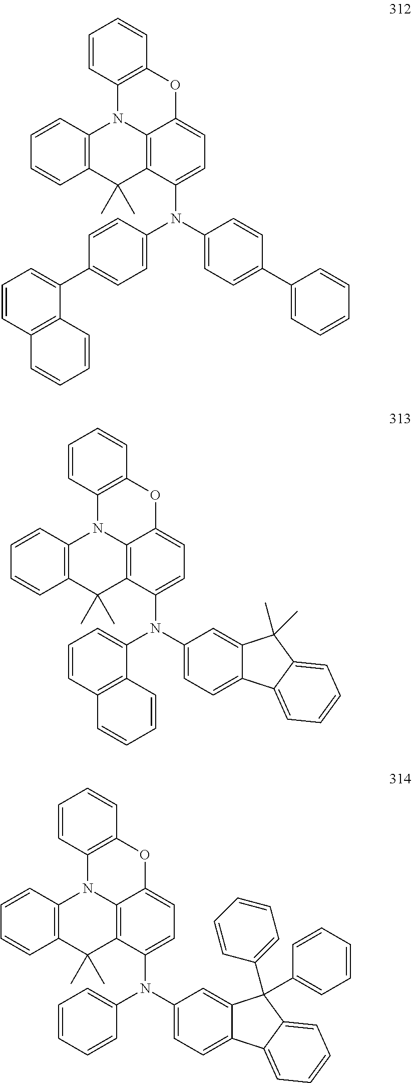 [SMILES: CC1(C)c2ccccc2N2c3ccccc3Oc3ccc(N(c4ccc(-c5ccccc5)cc4)c4ccc(-c5cccc6ccccc56)cc4)c1c32 ; CC1(C)c2ccccc2-c2ccc(N(c3ccc4c5c3C(C)(C)c3ccccc3N5c3ccccc3O4)c3cccc4ccccc34)cc21 ; CC1(C)c2ccccc2N2c3ccccc3Oc3ccc(N(c4ccccc4)c4ccc5c(c4)C(c4ccccc4)(c4ccccc4)c4ccccc4-5)c1c32]

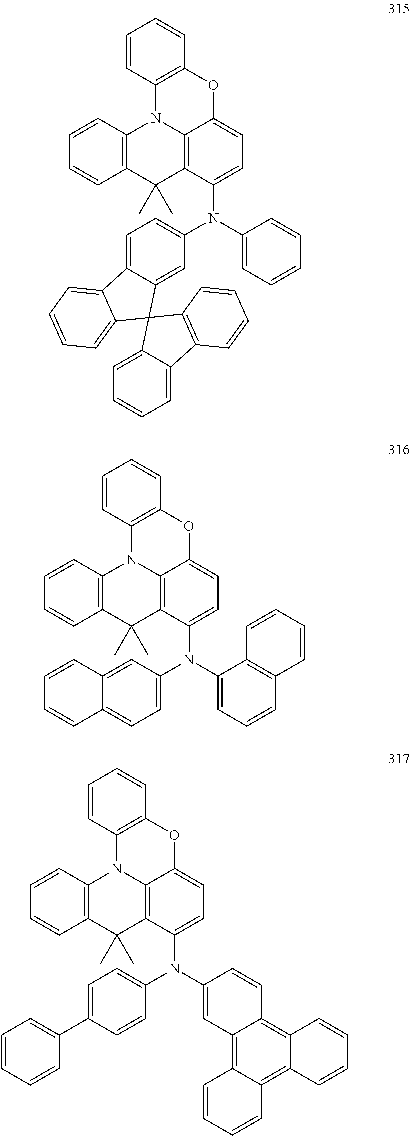

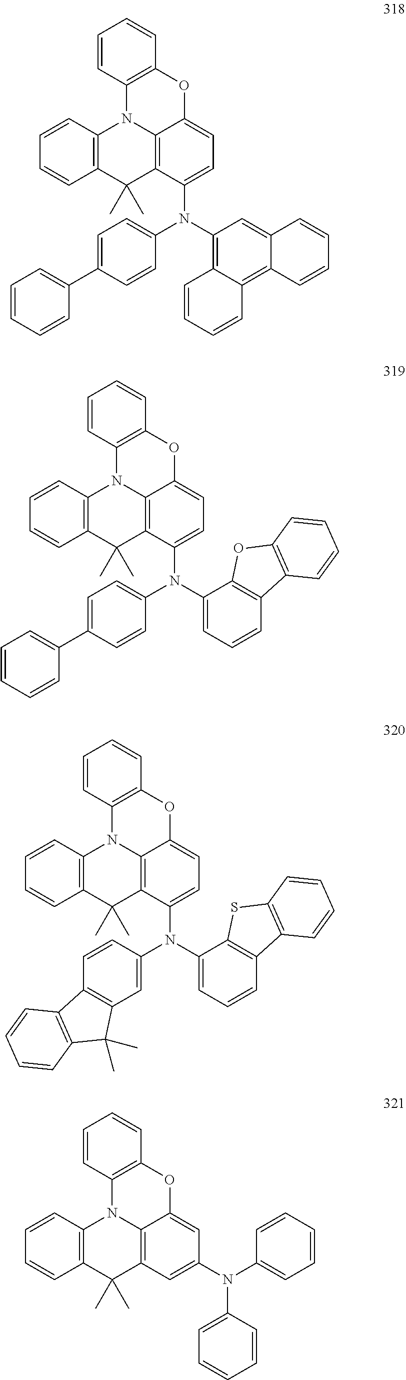

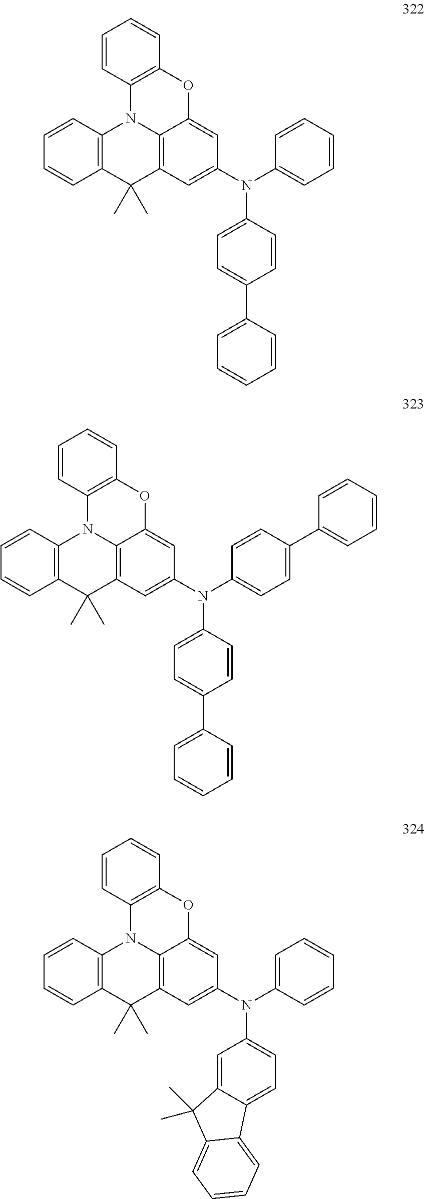 [SMILES: CC1(C)c2ccccc2N2c3ccccc3Oc3cc(N(c4ccccc4)c4ccc(-c5ccccc5)cc4)cc1c32 ; CC1(C)c2ccccc2N2c3ccccc3Oc3cc(N(c4ccc(-c5ccccc5)cc4)c4ccc(-c5ccccc5)cc4)cc1c32 ; CC1(C)c2ccccc2-c2ccc(N(c3ccccc3)c3cc4c5c(c3)C(C)(C)c3ccccc3N5c3ccccc3O4)cc21]

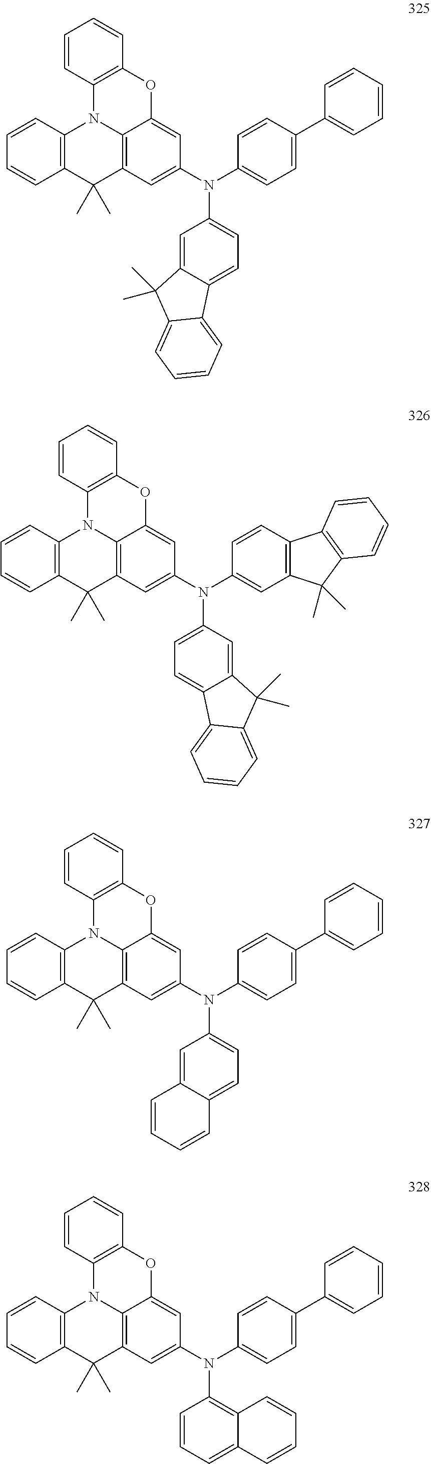 [SMILES: CC1(C)c2ccccc2-c2ccc(N(c3ccc(-c4ccccc4)cc3)c3cc4c5c(c3)C(C)(C)c3ccccc3N5c3ccccc3O4)cc21 ; CC1(C)c2ccccc2-c2ccc(N(c3ccc4c(c3)C(C)(C)c3ccccc3-4)c3cc4c5c(c3)C(C)(C)c3ccccc3N5c3ccccc3O4)cc21 ; CC1(C)c2ccccc2N2c3ccccc3Oc3cc(N(c4ccc(-c5ccccc5)cc4)c4ccc5ccccc5c4)cc1c32 ; CC1(C)c2ccccc2N2c3ccccc3Oc3cc(N(c4ccc(-c5ccccc5)cc4)c4cccc5ccccc45)cc1c32]

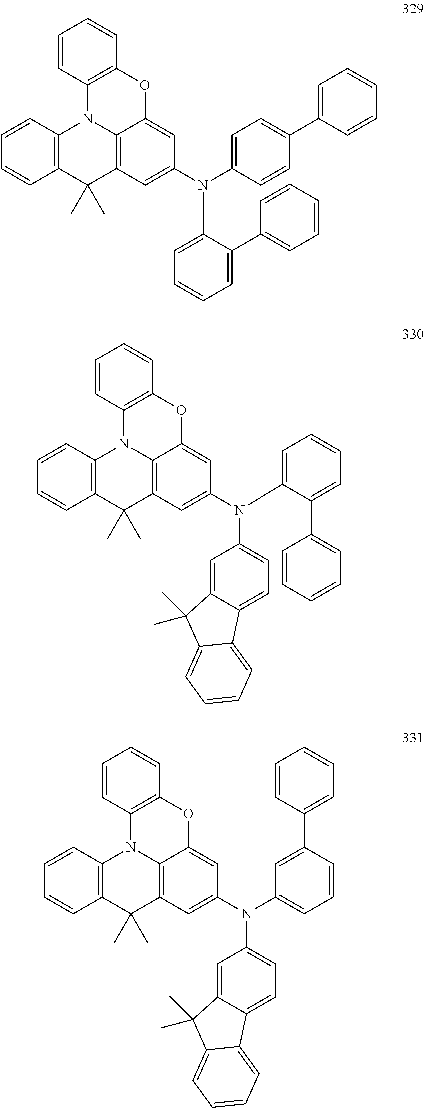 [SMILES: CC1(C)c2ccccc2N2c3ccccc3Oc3cc(N(c4ccc(-c5ccccc5)cc4)c4ccccc4-c4ccccc4)cc1c32 ; CC1(C)c2ccccc2-c2ccc(N(c3cc4c5c(c3)C(C)(C)c3ccccc3N5c3ccccc3O4)c3ccccc3-c3ccccc3)cc21 ; CC1(C)c2ccccc2-c2ccc(N(c3cccc(-c4ccccc4)c3)c3cc4c5c(c3)C(C)(C)c3ccccc3N5c3ccccc3O4)cc21]

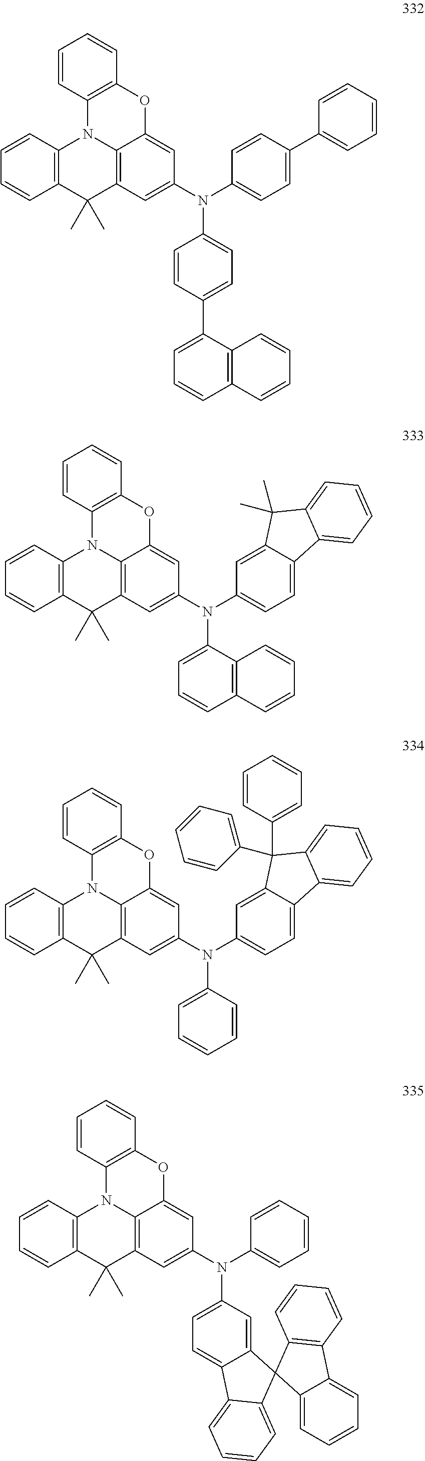

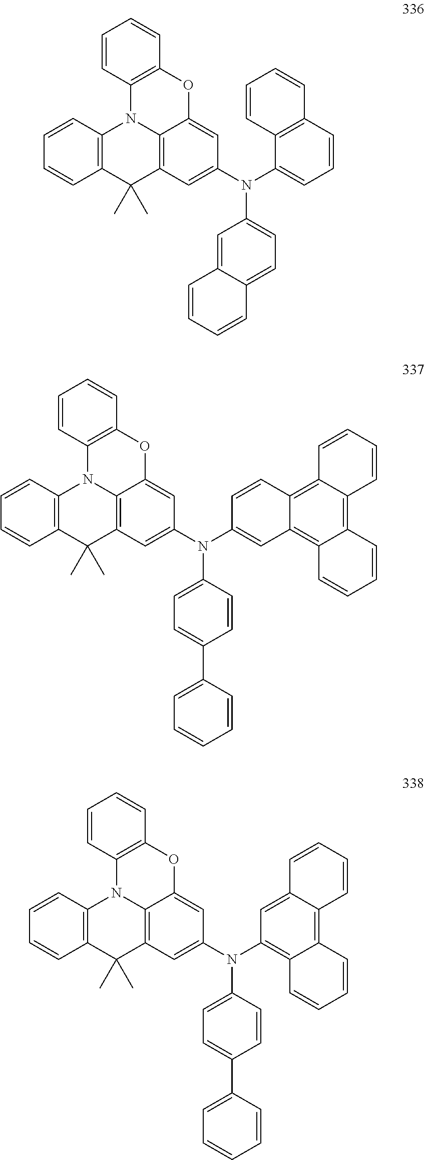 [SMILES: CC1(C)c2ccccc2N2c3ccccc3Oc3cc(N(c4ccc5ccccc5c4)c4cccc5ccccc45)cc1c32 ; CC1(C)c2ccccc2N2c3ccccc3Oc3cc(N(c4ccc(-c5ccccc5)cc4)c4ccc5c6ccccc6c6ccccc6c5c4)cc1c32 ; CC1(C)c2ccccc2N2c3ccccc3Oc3cc(N(c4ccc(-c5ccccc5)cc4)c4cc5ccccc5c5ccccc45)cc1c32]

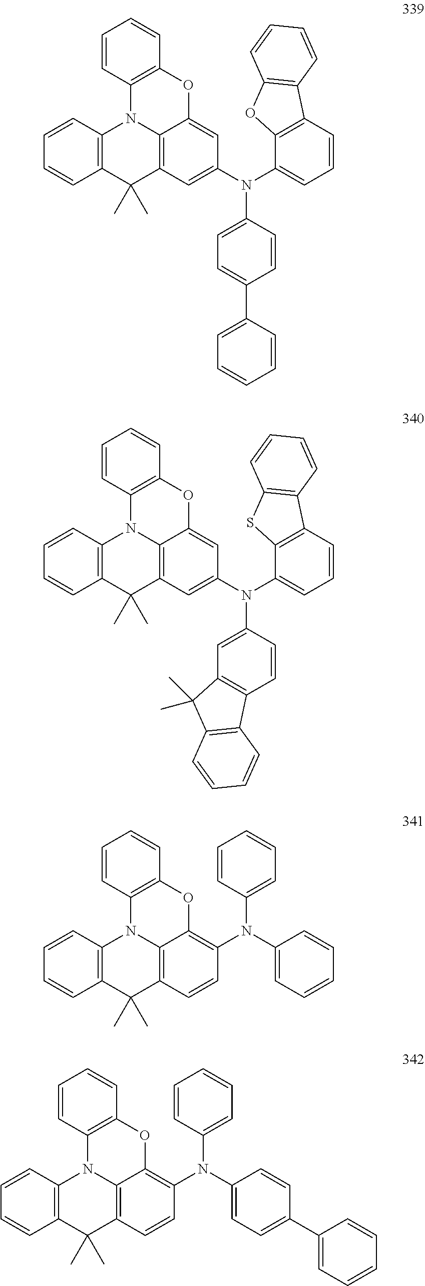

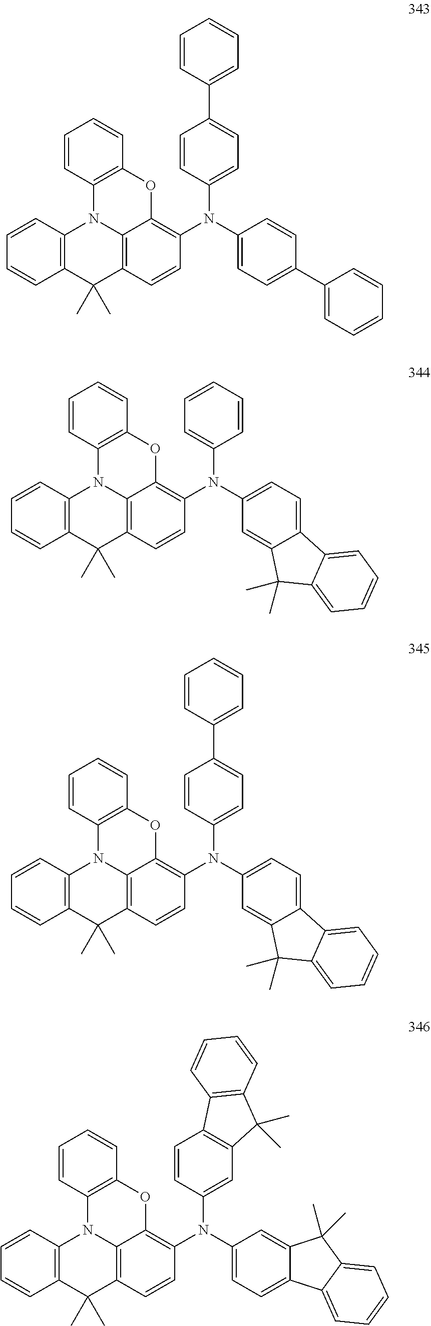 [SMILES: CC1(C)c2ccccc2N2c3ccccc3Oc3c(N(c4ccc(-c5ccccc5)cc4)c4ccc(-c5ccccc5)cc4)ccc1c32 ; CC1(C)c2ccccc2-c2ccc(N(c3ccccc3)c3ccc4c5c3Oc3ccccc3N5c3ccccc3C4(C)C)cc21 ; CC1(C)c2ccccc2-c2ccc(N(c3ccc(-c4ccccc4)cc3)c3ccc4c5c3Oc3ccccc3N5c3ccccc3C4(C)C)cc21 ; CC1(C)c2ccccc2-c2ccc(N(c3ccc4c(c3)C(C)(C)c3ccccc3-4)c3ccc4c5c3Oc3ccccc3N5c3ccccc3C4(C)C)cc21]

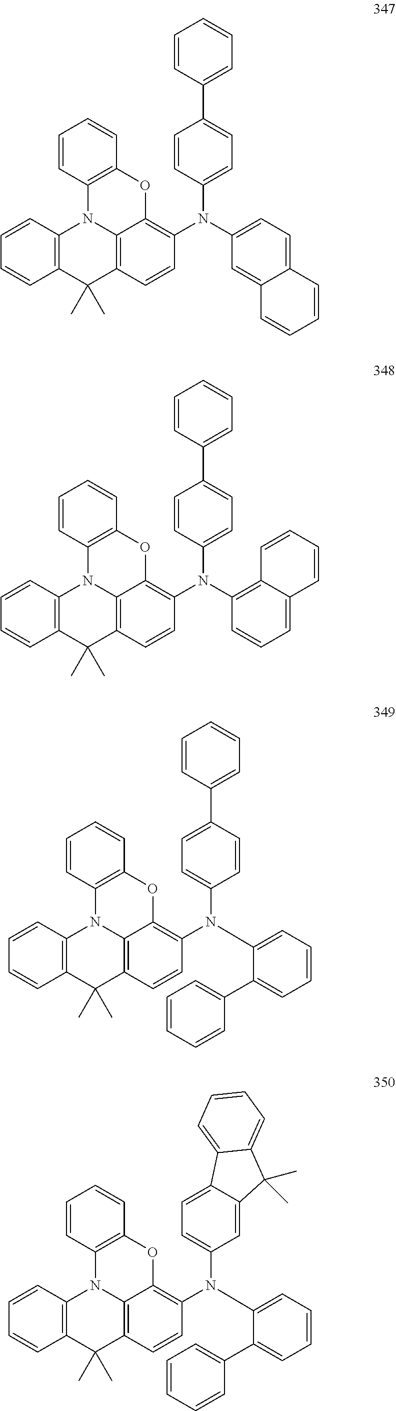 [SMILES: CC1(C)c2ccccc2N2c3ccccc3Oc3c(N(c4ccc(-c5ccccc5)cc4)c4ccc5ccccc5c4)ccc1c32 ; CC1(C)c2ccccc2N2c3ccccc3Oc3c(N(c4ccc(-c5ccccc5)cc4)c4cccc5ccccc45)ccc1c32 ; CC1(C)c2ccccc2N2c3ccccc3Oc3c(N(c4ccc(-c5ccccc5)cc4)c4ccccc4-c4ccccc4)ccc1c32 ; CC1(C)c2ccccc2-c2ccc(N(c3ccccc3-c3ccccc3)c3ccc4c5c3Oc3ccccc3N5c3ccccc3C4(C)C)cc21]

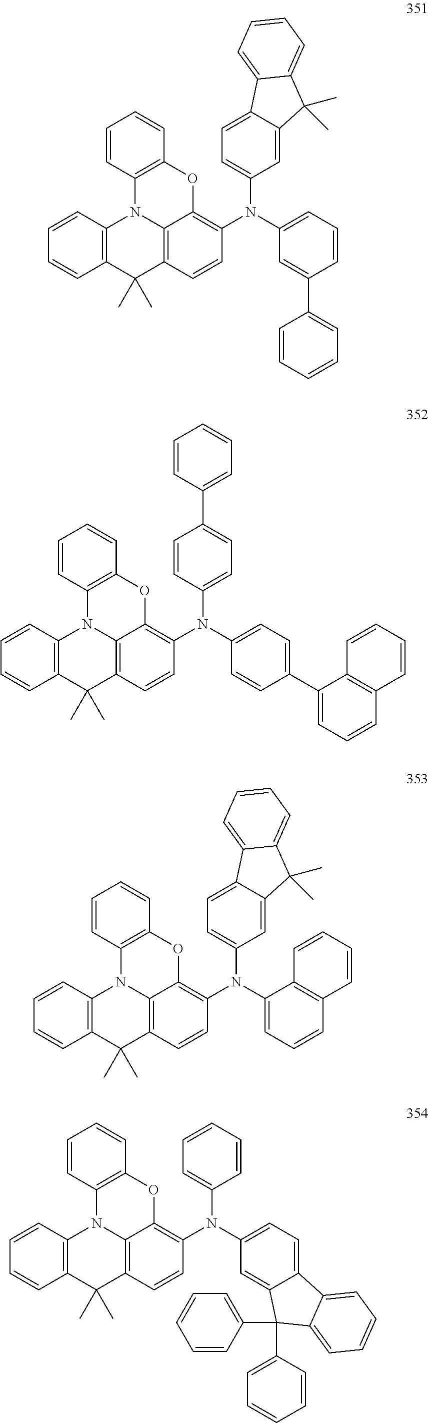

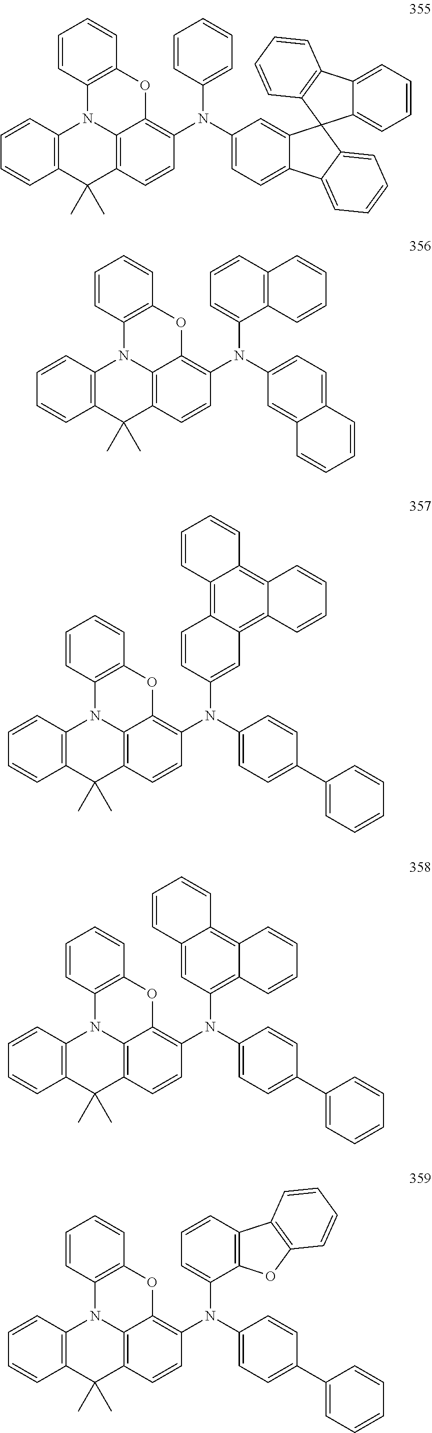

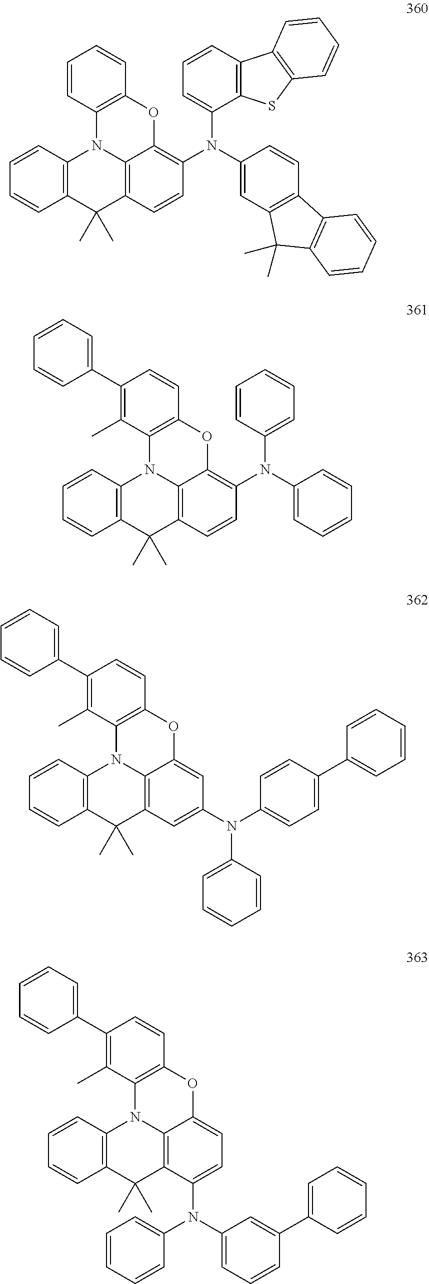 [SMILES: CC1(C)c2ccccc2-c2ccc(N(c3ccc4c5c3Oc3ccccc3N5c3ccccc3C4(C)C)c3cccc4c3sc3ccccc34)cc21 ; Cc1c(-c2ccccc2)ccc2c1N1c3ccccc3C(C)(C)c3ccc(N(c4ccccc4)c4ccccc4)c(c31)O2 ; Cc1c(-c2ccccc2)ccc2c1N1c3ccccc3C(C)(C)c3cc(N(c4ccccc4)c4ccc(-c5ccccc5)cc4)cc(c31)O2 ; Cc1c(-c2ccccc2)ccc2c1N1c3ccccc3C(C)(C)c3c(N(c4ccccc4)c4cccc(-c5ccccc5)c4)ccc(c31)O2]

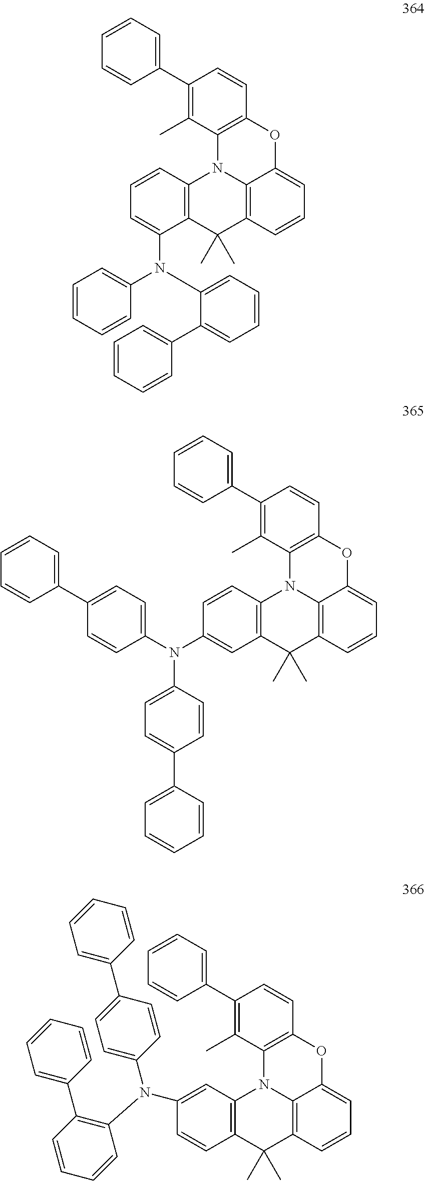 [SMILES: Cc1c(-c2ccccc2)ccc2c1N1c3cccc(N(c4ccccc4)c4ccccc4-c4ccccc4)c3C(C)(C)c3cccc(c31)O2 ; Cc1c(-c2ccccc2)ccc2c1N1c3ccc(N(c4ccc(-c5ccccc5)cc4)c4ccc(-c5ccccc5)cc4)cc3C(C)(C)c3cccc(c31)O2 ; Cc1c(-c2ccccc2)ccc2c1N1c3cc(N(c4ccc(-c5ccccc5)cc4)c4ccccc4-c4ccccc4)ccc3C(C)(C)c3cccc(c31)O2]

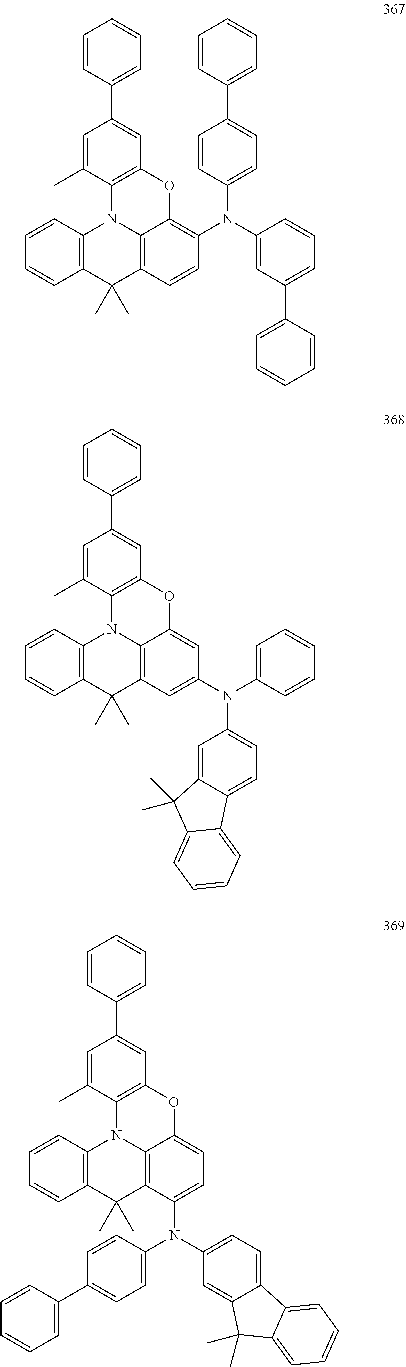 [SMILES: Cc1cc(-c2ccccc2)cc2c1N1c3ccccc3C(C)(C)c3ccc(N(c4ccc(-c5ccccc5)cc4)c4cccc(-c5ccccc5)c4)c(c31)O2 ; Cc1cc(-c2ccccc2)cc2c1N1c3ccccc3C(C)(C)c3cc(N(c4ccccc4)c4ccc5c(c4)C(C)(C)c4ccccc4-5)cc(c31)O2 ; Cc1cc(-c2ccccc2)cc2c1N1c3ccccc3C(C)(C)c3c(N(c4ccc(-c5ccccc5)cc4)c4ccc5c(c4)C(C)(C)c4ccccc4-5)ccc(c31)O2]

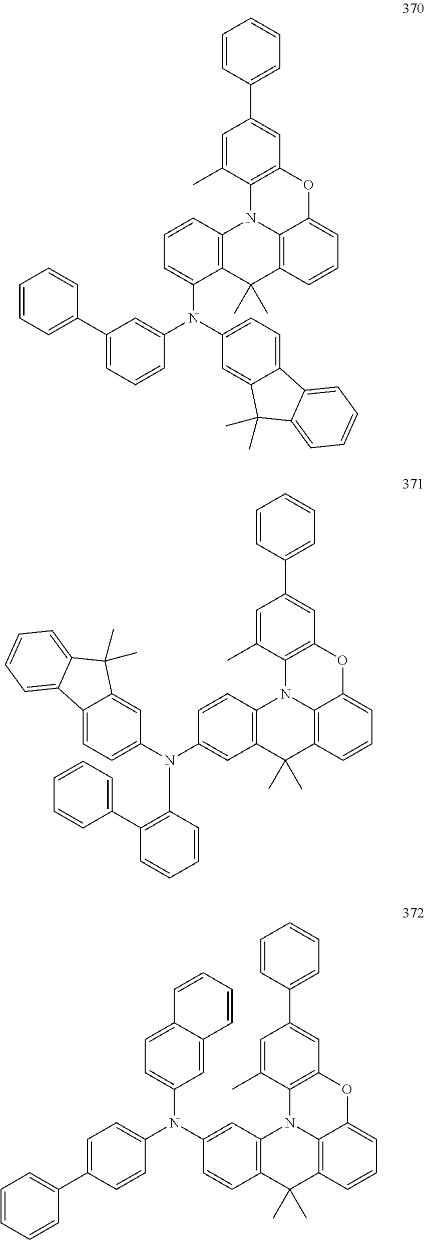 [SMILES: Cc1cc(-c2ccccc2)cc2c1N1c3cccc(N(c4cccc(-c5ccccc5)c4)c4ccc5c(c4)C(C)(C)c4ccccc4-5)c3C(C)(C)c3cccc(c31)O2 ; Cc1cc(-c2ccccc2)cc2c1N1c3ccc(N(c4ccc5c(c4)C(C)(C)c4ccccc4-5)c4ccccc4-c4ccccc4)cc3C(C)(C)c3cccc(c31)O2 ; Cc1cc(-c2ccccc2)cc2c1N1c3cc(N(c4ccc(-c5ccccc5)cc4)c4ccc5ccccc5c4)ccc3C(C)(C)c3cccc(c31)O2]

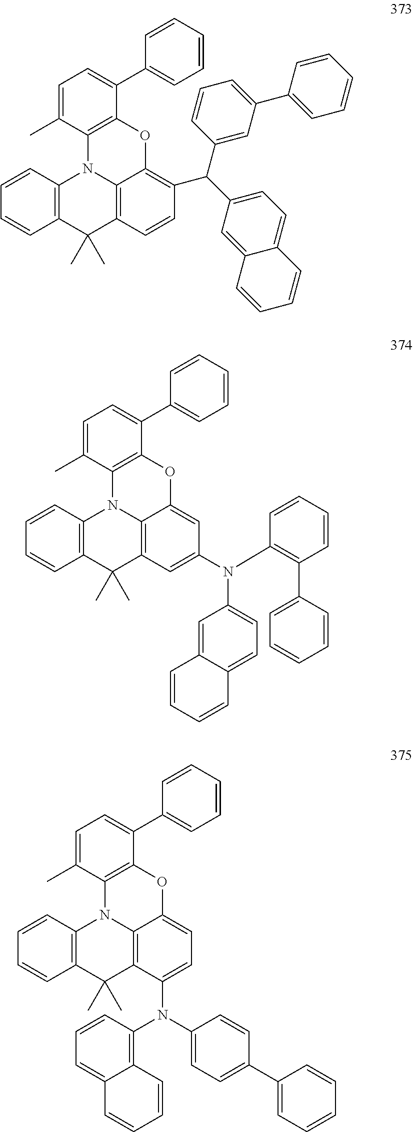

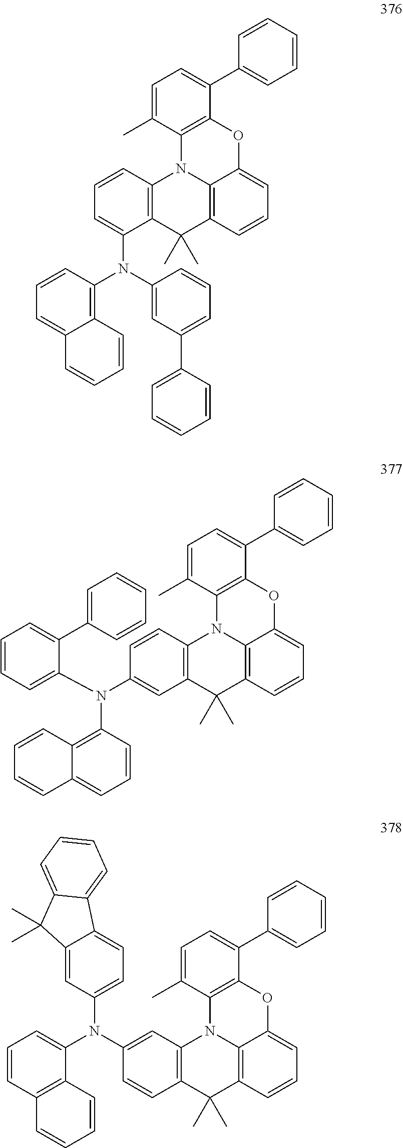

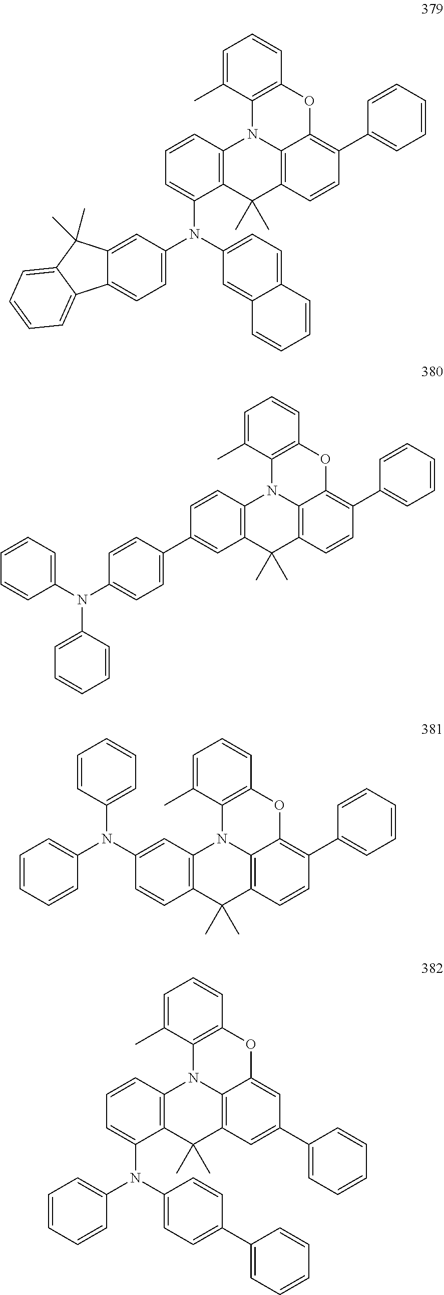

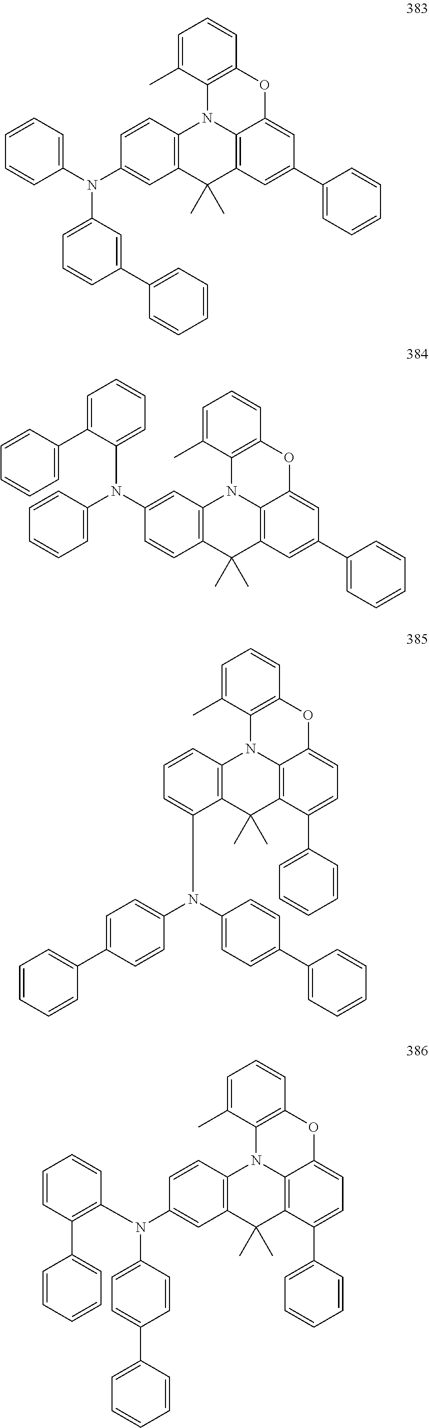 [SMILES: Cc1cccc2c1N1c3ccc(N(c4ccccc4)c4cccc(-c5ccccc5)c4)cc3C(C)(C)c3cc(-c4ccccc4)cc(c31)O2 ; Cc1cccc2c1N1c3cc(N(c4ccccc4)c4ccccc4-c4ccccc4)ccc3C(C)(C)c3cc(-c4ccccc4)cc(c31)O2 ; Cc1cccc2c1N1c3cccc(N(c4ccc(-c5ccccc5)cc4)c4ccc(-c5ccccc5)cc4)c3C(C)(C)c3c(-c4ccccc4)ccc(c31)O2 ; Cc1cccc2c1N1c3ccc(N(c4ccc(-c5ccccc5)cc4)c4ccccc4-c4ccccc4)cc3C(C)(C)c3c(-c4ccccc4)ccc(c31)O2]

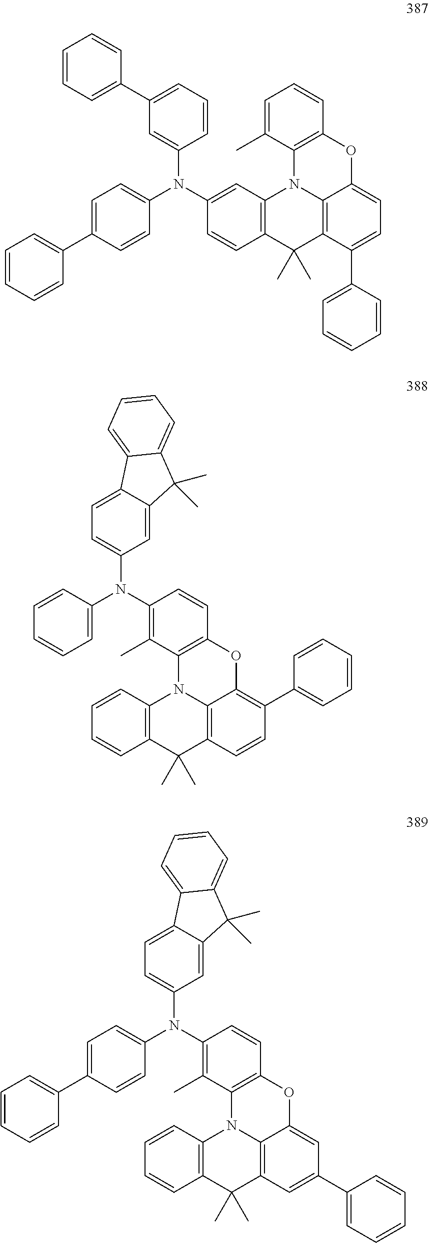 [SMILES: Cc1cccc2c1N1c3cc(N(c4ccc(-c5ccccc5)cc4)c4cccc(-c5ccccc5)c4)ccc3C(C)(C)c3c(-c4ccccc4)ccc(c31)O2 ; Cc1c(N(c2ccccc2)c2ccc3c(c2)C(C)(C)c2ccccc2-3)ccc2c1N1c3ccccc3C(C)(C)c3ccc(-c4ccccc4)c(c31)O2 ; Cc1c(N(c2ccc(-c3ccccc3)cc2)c2ccc3c(c2)C(C)(C)c2ccccc2-3)ccc2c1N1c3ccccc3C(C)(C)c3cc(-c4ccccc4)cc(c31)O2]

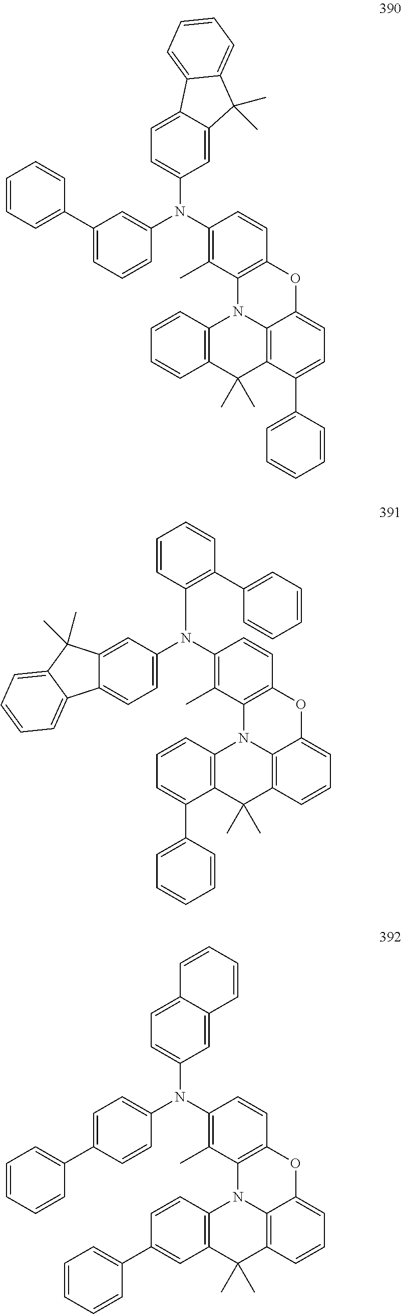 [SMILES: Cc1c(N(c2cccc(-c3ccccc3)c2)c2ccc3c(c2)C(C)(C)c2ccccc2-3)ccc2c1N1c3ccccc3C(C)(C)c3c(-c4ccccc4)ccc(c31)O2 ; Cc1c(N(c2ccc3c(c2)C(C)(C)c2ccccc2-3)c2ccccc2-c2ccccc2)ccc2c1N1c3cccc(-c4ccccc4)c3C(C)(C)c3cccc(c31)O2 ; Cc1c(N(c2ccc(-c3ccccc3)cc2)c2ccc3ccccc3c2)ccc2c1N1c3ccc(-c4ccccc4)cc3C(C)(C)c3cccc(c31)O2]

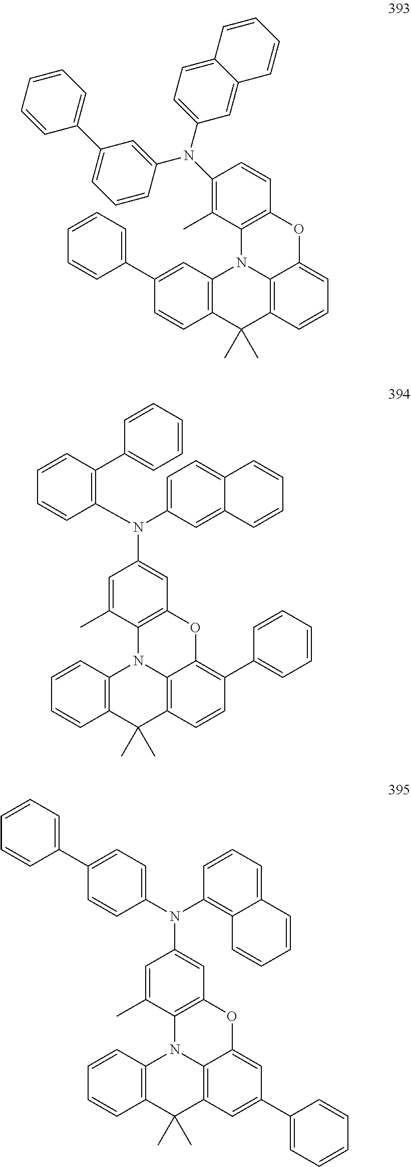 [SMILES: Cc1c(N(c2cccc(-c3ccccc3)c2)c2ccc3ccccc3c2)ccc2c1N1c3cc(-c4ccccc4)ccc3C(C)(C)c3cccc(c31)O2 ; Cc1cc(N(c2ccc3ccccc3c2)c2ccccc2-c2ccccc2)cc2c1N1c3ccccc3C(C)(C)c3ccc(-c4ccccc4)c(c31)O2 ; Cc1cc(N(c2ccc(-c3ccccc3)cc2)c2cccc3ccccc23)cc2c1N1c3ccccc3C(C)(C)c3cc(-c4ccccc4)cc(c31)O2]

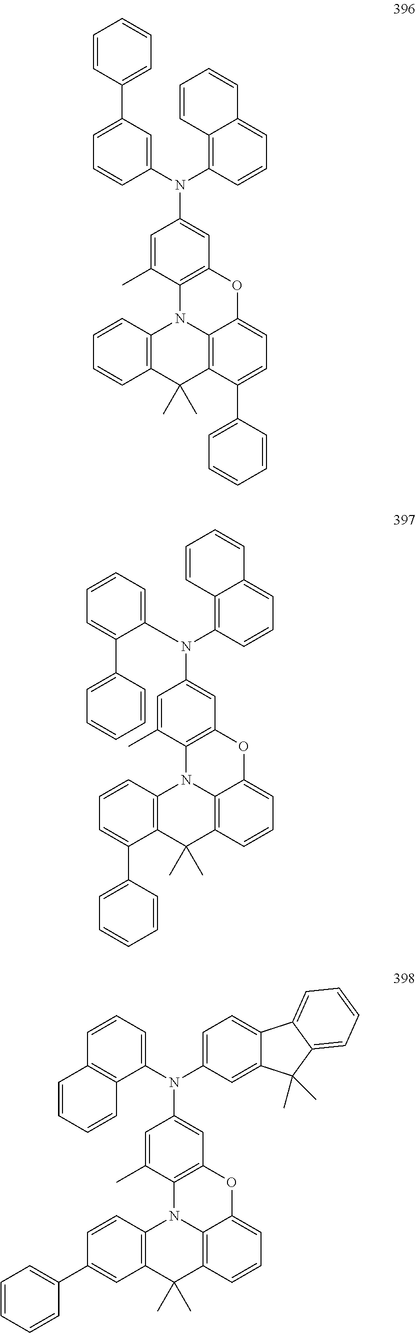 [SMILES: Cc1cc(N(c2cccc(-c3ccccc3)c2)c2cccc3ccccc23)cc2c1N1c3ccccc3C(C)(C)c3c(-c4ccccc4)ccc(c31)O2 ; Cc1cc(N(c2ccccc2-c2ccccc2)c2cccc3ccccc23)cc2c1N1c3cccc(-c4ccccc4)c3C(C)(C)c3cccc(c31)O2 ; Cc1cc(N(c2ccc3c(c2)C(C)(C)c2ccccc2-3)c2cccc3ccccc23)cc2c1N1c3ccc(-c4ccccc4)cc3C(C)(C)c3cccc(c31)O2]

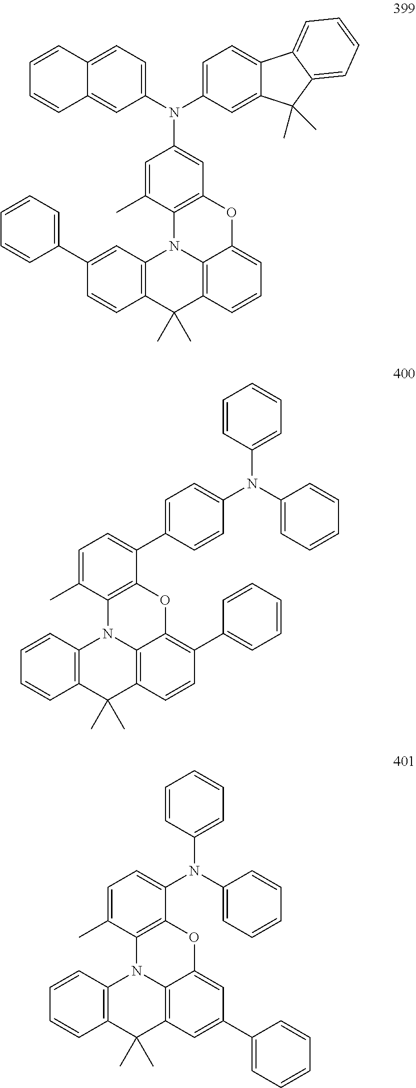 [SMILES: Cc1cc(N(c2ccc3c(c2)C(C)(C)c2ccccc2-3)c2ccc3ccccc3c2)cc2c1N1c3cc(-c4ccccc4)ccc3C(C)(C)c3cccc(c31)O2 ; Cc1ccc(-c2ccc(N(c3ccccc3)c3ccccc3)cc2)c2c1N1c3ccccc3C(C)(C)c3ccc(-c4ccccc4)c(c31)O2 ; Cc1ccc(N(c2ccccc2)c2ccccc2)c2c1N1c3ccccc3C(C)(C)c3cc(-c4ccccc4)cc(c31)O2]

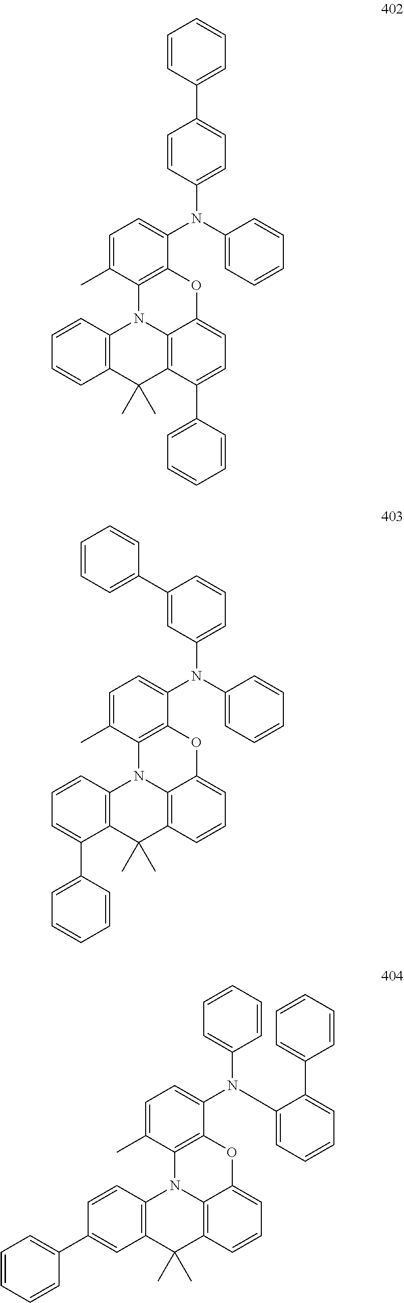 [SMILES: Cc1ccc(N(c2ccccc2)c2ccc(-c3ccccc3)cc2)c2c1N1c3ccccc3C(C)(C)c3c(-c4ccccc4)ccc(c31)O2 ; Cc1ccc(N(c2ccccc2)c2cccc(-c3ccccc3)c2)c2c1N1c3cccc(-c4ccccc4)c3C(C)(C)c3cccc(c31)O2 ; Cc1ccc(N(c2ccccc2)c2ccccc2-c2ccccc2)c2c1N1c3ccc(-c4ccccc4)cc3C(C)(C)c3cccc(c31)O2]

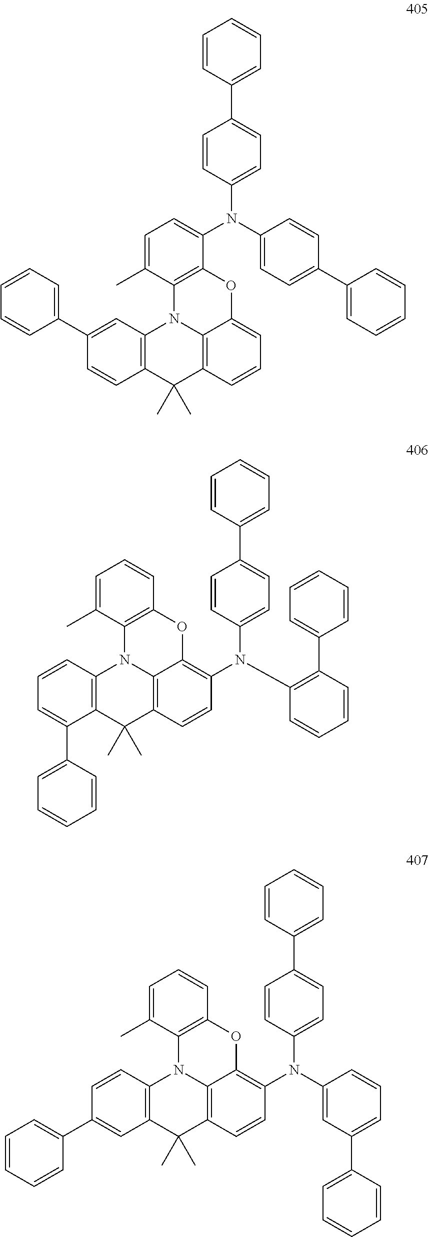 [SMILES: Cc1ccc(N(c2ccc(-c3ccccc3)cc2)c2ccc(-c3ccccc3)cc2)c2c1N1c3cc(-c4ccccc4)ccc3C(C)(C)c3cccc(c31)O2 ; Cc1cccc2c1N1c3cccc(-c4ccccc4)c3C(C)(C)c3ccc(N(c4ccc(-c5ccccc5)cc4)c4ccccc4-c4ccccc4)c(c31)O2 ; Cc1cccc2c1N1c3ccc(-c4ccccc4)cc3C(C)(C)c3ccc(N(c4ccc(-c5ccccc5)cc4)c4cccc(-c5ccccc5)c4)c(c31)O2]

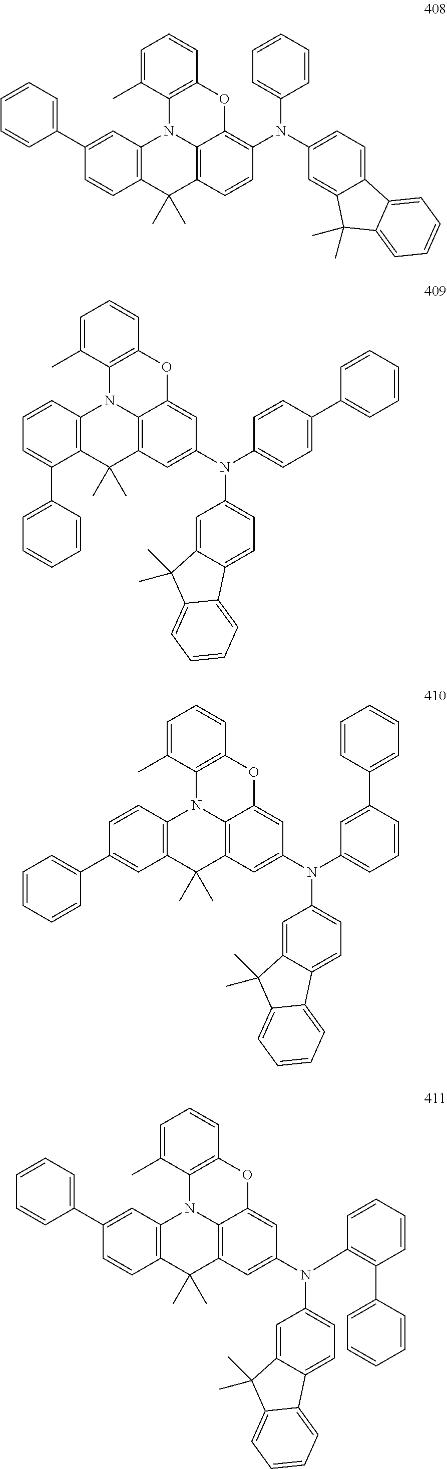

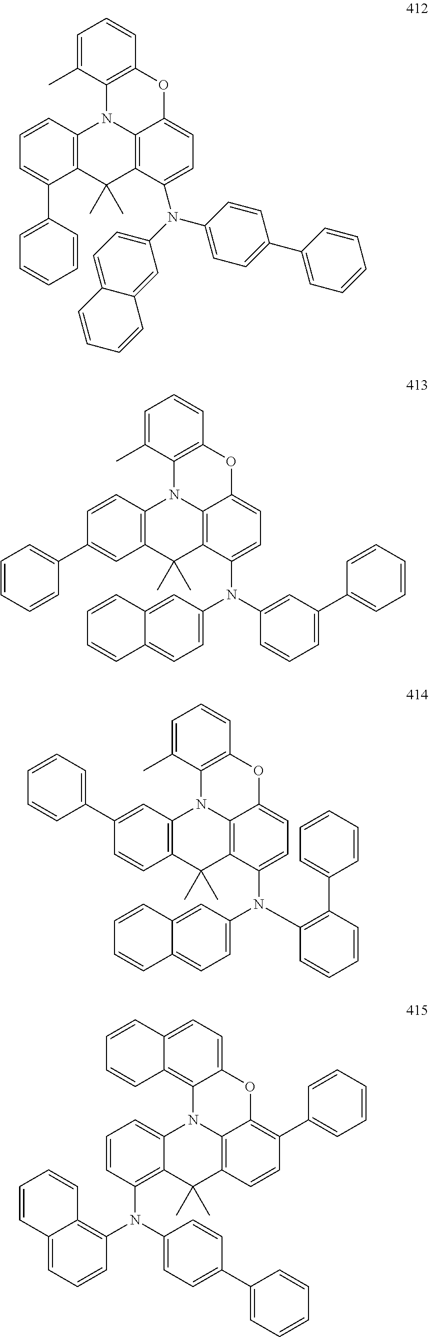 [SMILES: Cc1cccc2c1N1c3cccc(-c4ccccc4)c3C(C)(C)c3c(N(c4ccc(-c5ccccc5)cc4)c4ccc5ccccc5c4)ccc(c31)O2 ; Cc1cccc2c1N1c3ccc(-c4ccccc4)cc3C(C)(C)c3c(N(c4cccc(-c5ccccc5)c4)c4ccc5ccccc5c4)ccc(c31)O2 ; Cc1cccc2c1N1c3cc(-c4ccccc4)ccc3C(C)(C)c3c(N(c4ccc5ccccc5c4)c4ccccc4-c4ccccc4)ccc(c31)O2 ; CC1(C)c2ccc(-c3ccccc3)c3c2N(c2cccc(N(c4ccc(-c5ccccc5)cc4)c4cccc5ccccc45)c21)c1c(ccc2ccccc12)O3]

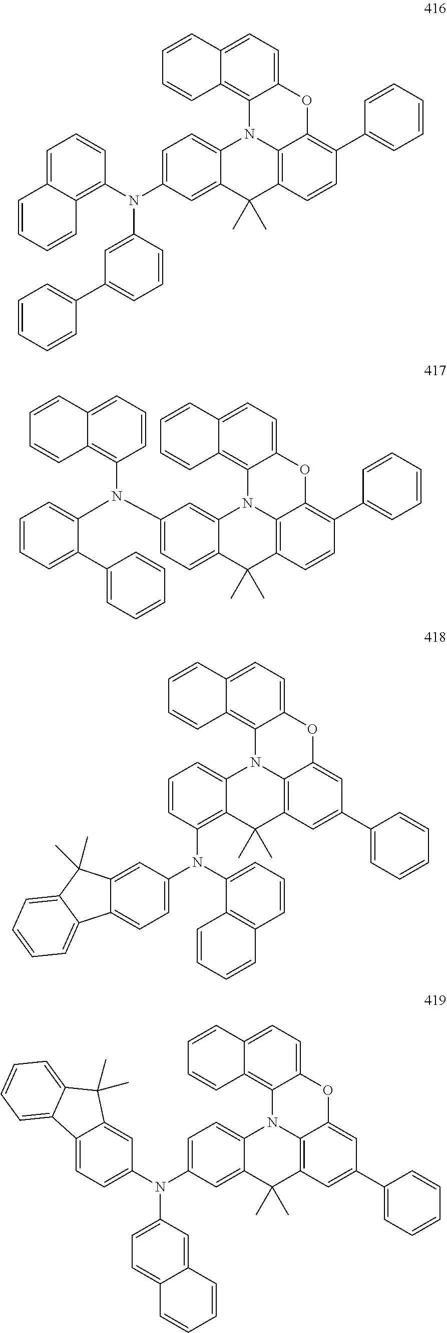 [SMILES: CC1(C)c2cc(N(c3cccc(-c4ccccc4)c3)c3cccc4ccccc34)ccc2N2c3c1ccc(-c1ccccc1)c3Oc1ccc3ccccc3c12 ; CC1(C)c2ccc(N(c3ccccc3-c3ccccc3)c3cccc4ccccc34)cc2N2c3c1ccc(-c1ccccc1)c3Oc1ccc3ccccc3c12 ; CC1(C)c2ccccc2-c2ccc(N(c3cccc4c3C(C)(C)c3cc(-c5ccccc5)cc5c3N4c3c(ccc4ccccc34)O5)c3cccc4ccccc34)cc21 ; CC1(C)c2ccccc2-c2ccc(N(c3ccc4c(c3)C(C)(C)c3cc(-c5ccccc5)cc5c3N4c3c(ccc4ccccc34)O5)c3ccc4ccccc4c3)cc21]

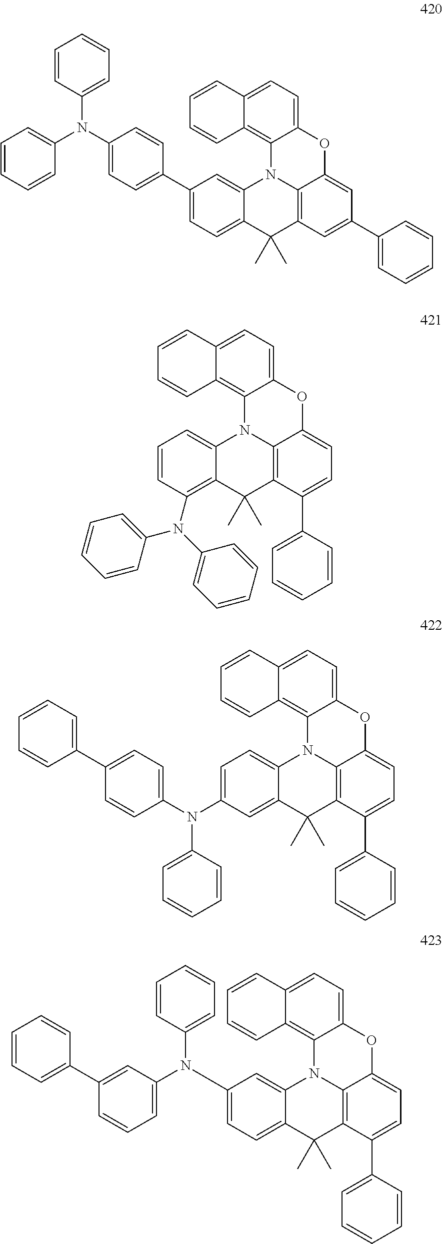 [SMILES: CC1(C)c2ccc(-c3ccc(N(c4ccccc4)c4ccccc4)cc3)cc2N2c3c(cc(-c4ccccc4)cc31)Oc1ccc3ccccc3c12 ; CC1(C)c2c(N(c3ccccc3)c3ccccc3)cccc2N2c3c(ccc(-c4ccccc4)c31)Oc1ccc3ccccc3c12 ; CC1(C)c2cc(N(c3ccccc3)c3ccc(-c4ccccc4)cc3)ccc2N2c3c(ccc(-c4ccccc4)c31)Oc1ccc3ccccc3c12 ; CC1(C)c2ccc(N(c3ccccc3)c3cccc(-c4ccccc4)c3)cc2N2c3c(ccc(-c4ccccc4)c31)Oc1ccc3ccccc3c12]

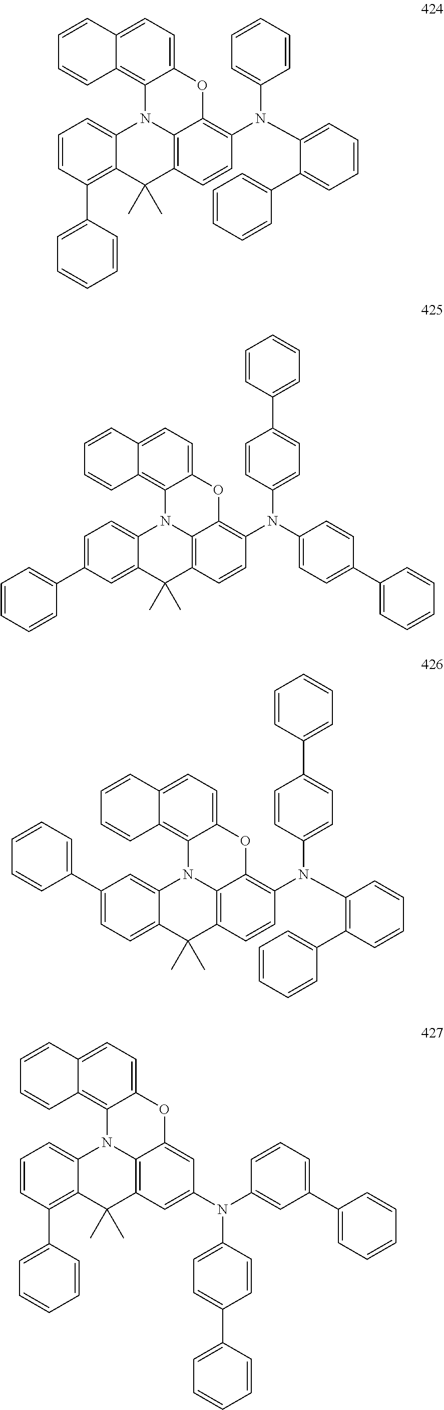 [SMILES: CC1(C)c2ccc(N(c3ccccc3)c3ccccc3-c3ccccc3)c3c2N(c2cccc(-c4ccccc4)c21)c1c(ccc2ccccc12)O3 ; CC1(C)c2cc(-c3ccccc3)ccc2N2c3c1ccc(N(c1ccc(-c4ccccc4)cc1)c1ccc(-c4ccccc4)cc1)c3Oc1ccc3ccccc3c12 ; CC1(C)c2ccc(-c3ccccc3)cc2N2c3c1ccc(N(c1ccc(-c4ccccc4)cc1)c1ccccc1-c1ccccc1)c3Oc1ccc3ccccc3c12 ; CC1(C)c2cc(N(c3ccc(-c4ccccc4)cc3)c3cccc(-c4ccccc4)c3)cc3c2N(c2cccc(-c4ccccc4)c21)c1c(ccc2ccccc12)O3]

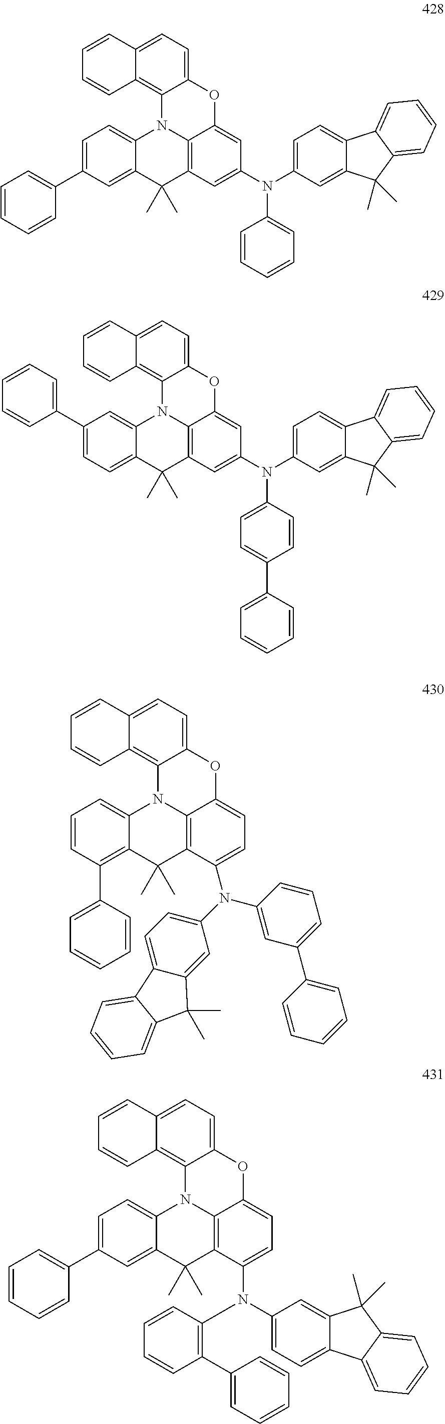

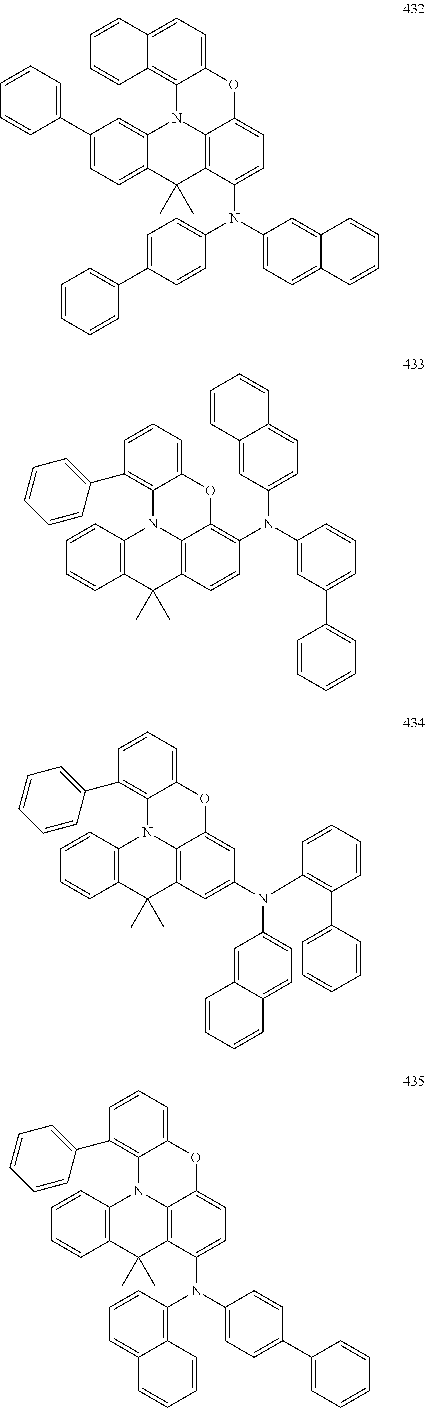 [SMILES: CC1(C)c2ccc(-c3ccccc3)cc2N2c3c(ccc(N(c4ccc(-c5ccccc5)cc4)c4ccc5ccccc5c4)c31)Oc1ccc3ccccc3c12 ; CC1(C)c2ccccc2N2c3c(cccc3-c3ccccc3)Oc3c(N(c4cccc(-c5ccccc5)c4)c4ccc5ccccc5c4)ccc1c32 ; CC1(C)c2ccccc2N2c3c(cccc3-c3ccccc3)Oc3cc(N(c4ccc5ccccc5c4)c4ccccc4-c4ccccc4)cc1c32 ; CC1(C)c2ccccc2N2c3c(cccc3-c3ccccc3)Oc3ccc(N(c4ccc(-c5ccccc5)cc4)c4cccc5ccccc45)c1c32]

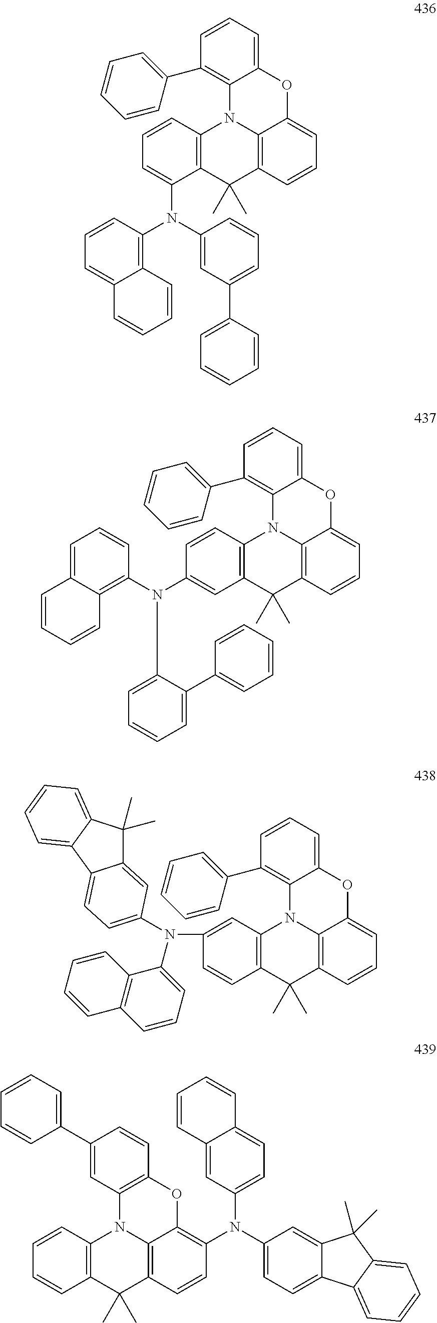 [SMILES: CC1(C)c2cccc3c2N(c2cccc(N(c4cccc(-c5ccccc5)c4)c4cccc5ccccc45)c21)c1c(cccc1-c1ccccc1)O3 ; CC1(C)c2cc(N(c3ccccc3-c3ccccc3)c3cccc4ccccc34)ccc2N2c3c(cccc3-c3ccccc3)Oc3cccc1c32 ; CC1(C)c2ccccc2-c2ccc(N(c3ccc4c(c3)N3c5c(cccc5-c5ccccc5)Oc5cccc(c53)C4(C)C)c3cccc4ccccc34)cc21 ; CC1(C)c2ccccc2-c2ccc(N(c3ccc4ccccc4c3)c3ccc4c5c3Oc3ccc(-c6ccccc6)cc3N5c3ccccc3C4(C)C)cc21]

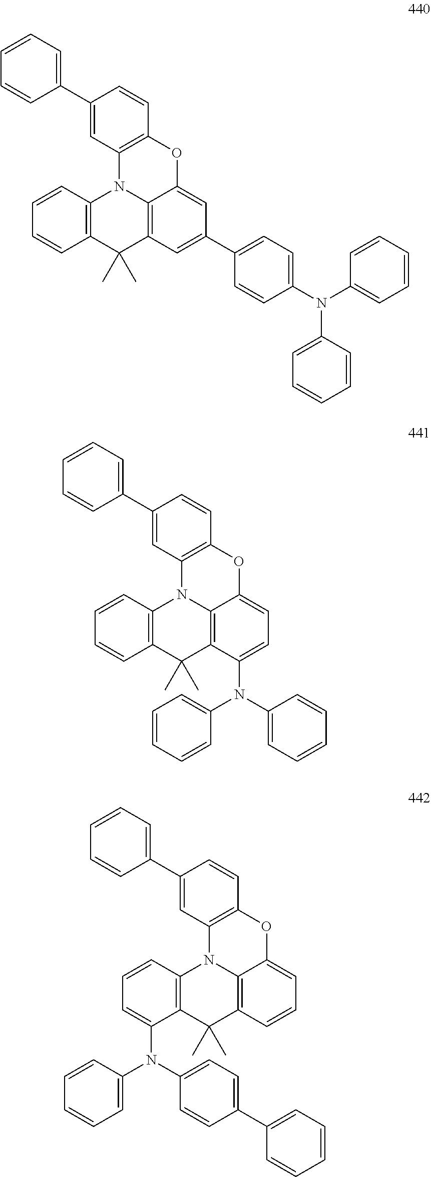 [SMILES: CC1(C)c2ccccc2N2c3cc(-c4ccccc4)ccc3Oc3cc(-c4ccc(N(c5ccccc5)c5ccccc5)cc4)cc1c32 ; CC1(C)c2ccccc2N2c3cc(-c4ccccc4)ccc3Oc3ccc(N(c4ccccc4)c4ccccc4)c1c32 ; CC1(C)c2cccc3c2N(c2cc(-c4ccccc4)ccc2O3)c2cccc(N(c3ccccc3)c3ccc(-c4ccccc4)cc3)c21]

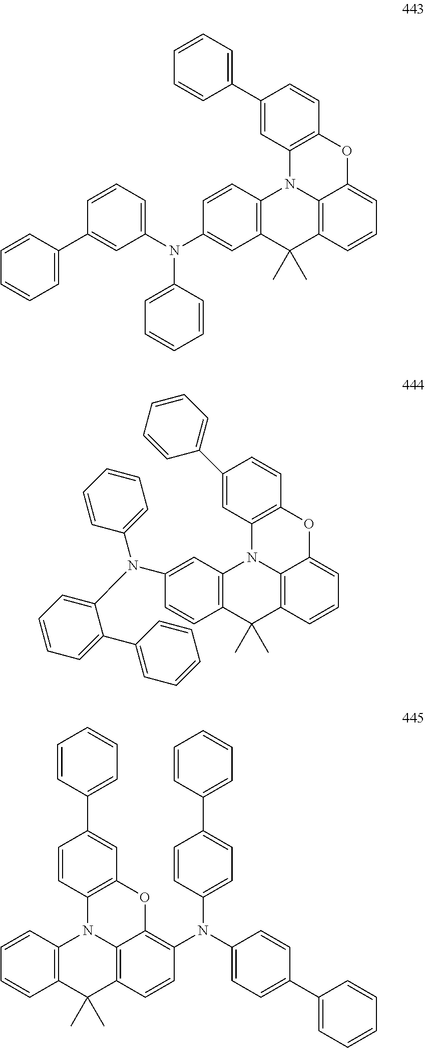

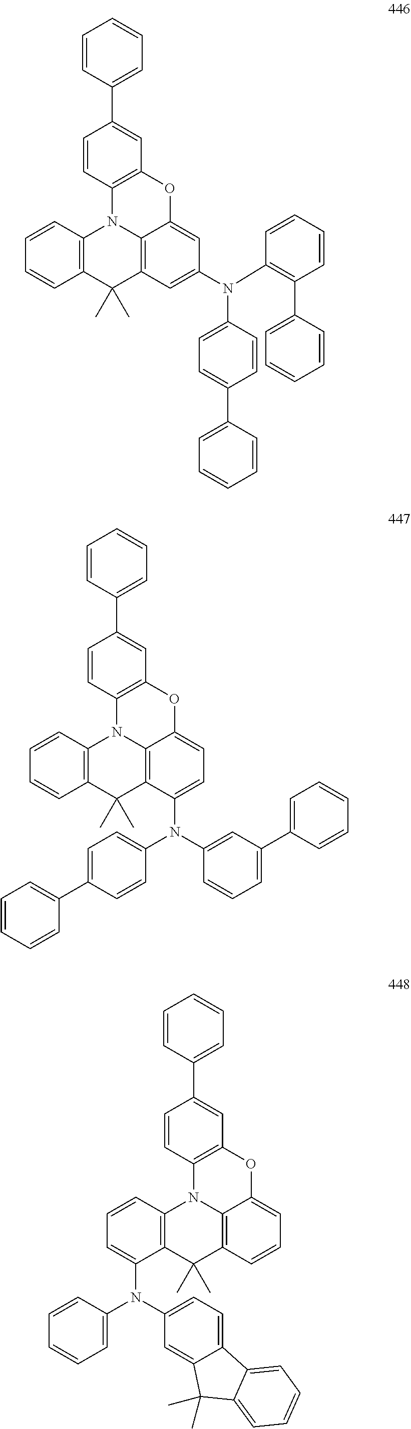 [SMILES: CC1(C)c2ccccc2N2c3ccc(-c4ccccc4)cc3Oc3cc(N(c4ccc(-c5ccccc5)cc4)c4ccccc4-c4ccccc4)cc1c32 ; CC1(C)c2ccccc2N2c3ccc(-c4ccccc4)cc3Oc3ccc(N(c4ccc(-c5ccccc5)cc4)c4cccc(-c5ccccc5)c4)c1c32 ; CC1(C)c2ccccc2-c2ccc(N(c3ccccc3)c3cccc4c3C(C)(C)c3cccc5c3N4c3ccc(-c4ccccc4)cc3O5)cc21]

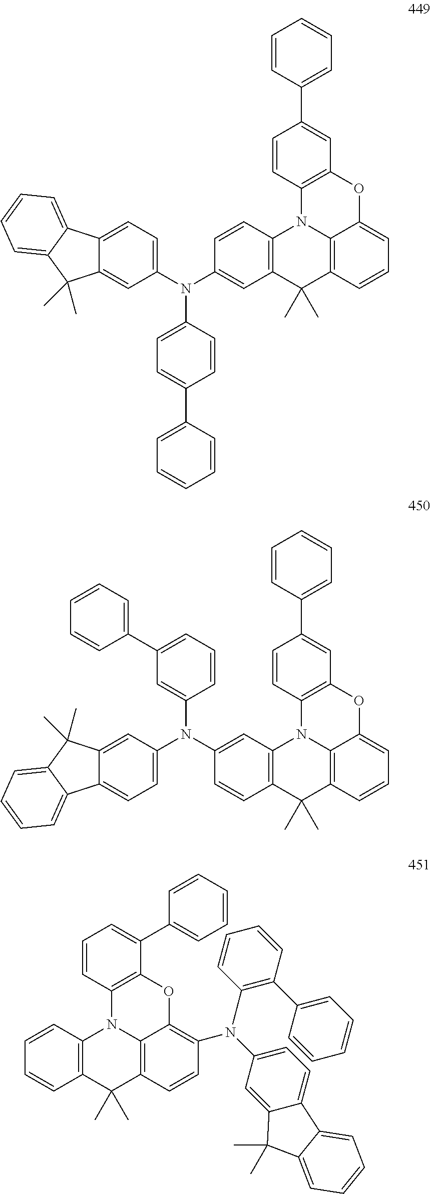 [SMILES: CC1(C)c2ccccc2-c2ccc(N(c3ccc(-c4ccccc4)cc3)c3ccc4c(c3)C(C)(C)c3cccc5c3N4c3ccc(-c4ccccc4)cc3O5)cc21 ; CC1(C)c2ccccc2-c2ccc(N(c3cccc(-c4ccccc4)c3)c3ccc4c(c3)N3c5ccc(-c6ccccc6)cc5Oc5cccc(c53)C4(C)C)cc21 ; CC1(C)c2ccccc2-c2ccc(N(c3ccccc3-c3ccccc3)c3ccc4c5c3Oc3c(-c6ccccc6)cccc3N5c3ccccc3C4(C)C)cc21]

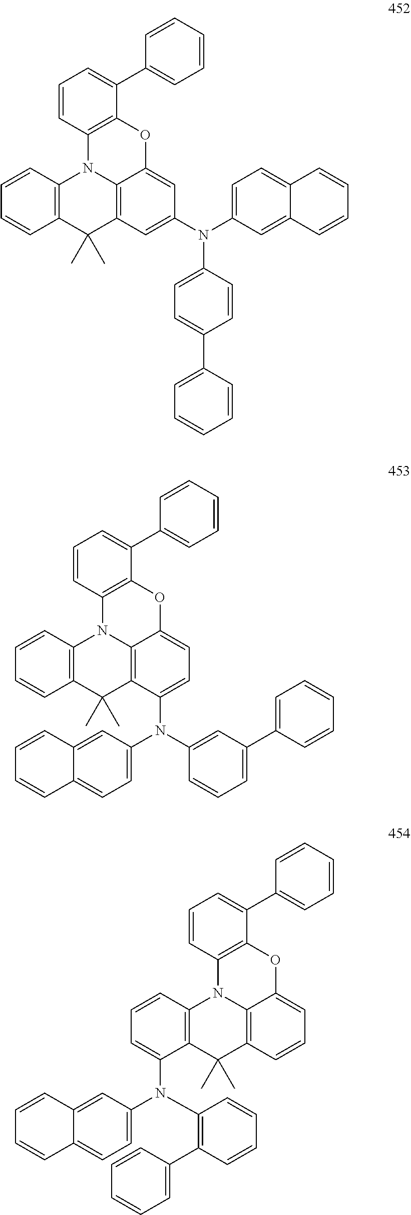 [SMILES: CC1(C)c2ccccc2N2c3cccc(-c4ccccc4)c3Oc3cc(N(c4ccc(-c5ccccc5)cc4)c4ccc5ccccc5c4)cc1c32 ; CC1(C)c2ccccc2N2c3cccc(-c4ccccc4)c3Oc3ccc(N(c4cccc(-c5ccccc5)c4)c4ccc5ccccc5c4)c1c32 ; CC1(C)c2cccc3c2N(c2cccc(-c4ccccc4)c2O3)c2cccc(N(c3ccc4ccccc4c3)c3ccccc3-c3ccccc3)c21]

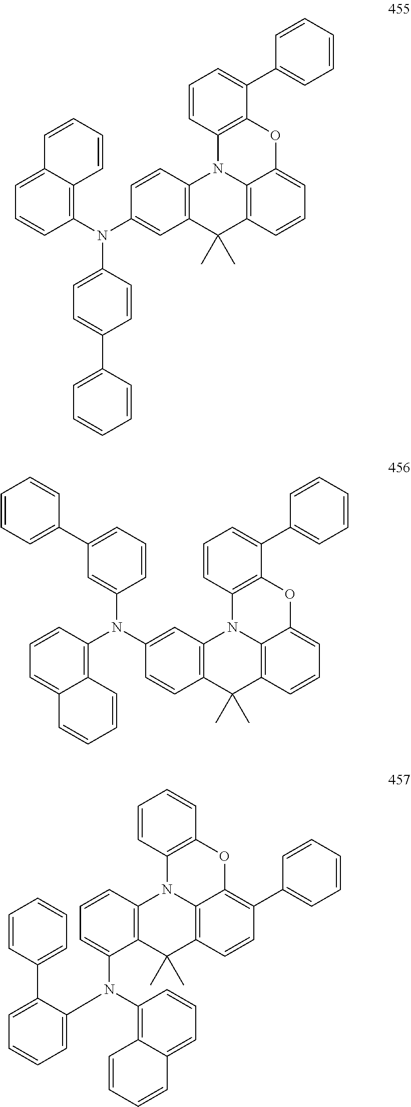 [SMILES: CC1(C)c2cc(N(c3ccc(-c4ccccc4)cc3)c3cccc4ccccc34)ccc2N2c3cccc(-c4ccccc4)c3Oc3cccc1c32 ; CC1(C)c2ccc(N(c3cccc(-c4ccccc4)c3)c3cccc4ccccc34)cc2N2c3cccc(-c4ccccc4)c3Oc3cccc1c32 ; CC1(C)c2ccc(-c3ccccc3)c3c2N(c2ccccc2O3)c2cccc(N(c3ccccc3-c3ccccc3)c3cccc4ccccc34)c21]

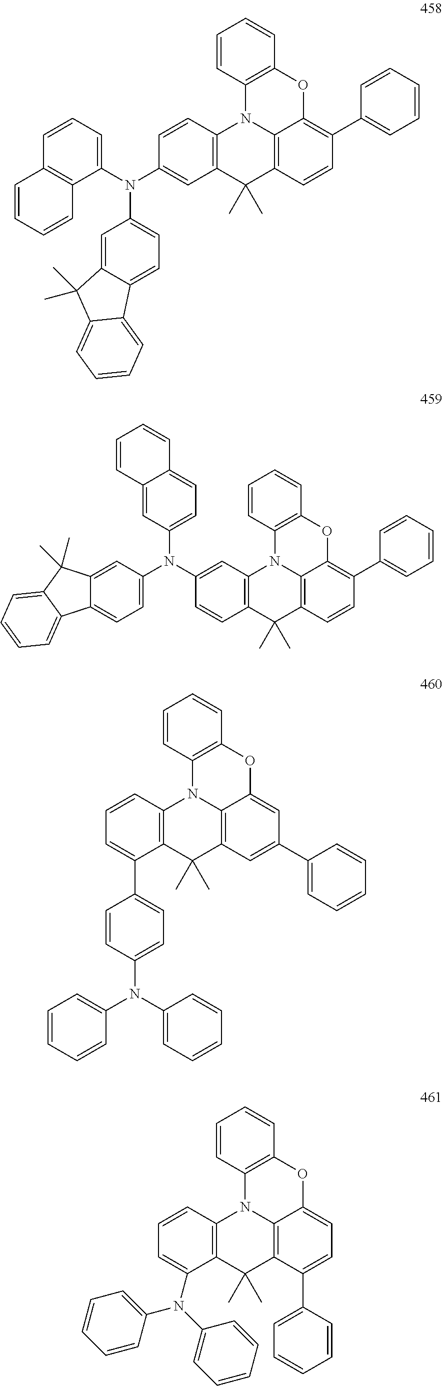 [SMILES: CC1(C)c2ccccc2-c2ccc(N(c3ccc4c(c3)C(C)(C)c3ccc(-c5ccccc5)c5c3N4c3ccccc3O5)c3cccc4ccccc34)cc21 ; CC1(C)c2ccccc2-c2ccc(N(c3ccc4c(c3)N3c5ccccc5Oc5c(-c6ccccc6)ccc(c53)C4(C)C)c3ccc4ccccc4c3)cc21 ; CC1(C)c2cc(-c3ccccc3)cc3c2N(c2ccccc2O3)c2cccc(-c3ccc(N(c4ccccc4)c4ccccc4)cc3)c21 ; CC1(C)c2c(N(c3ccccc3)c3ccccc3)cccc2N2c3ccccc3Oc3ccc(-c4ccccc4)c1c32]

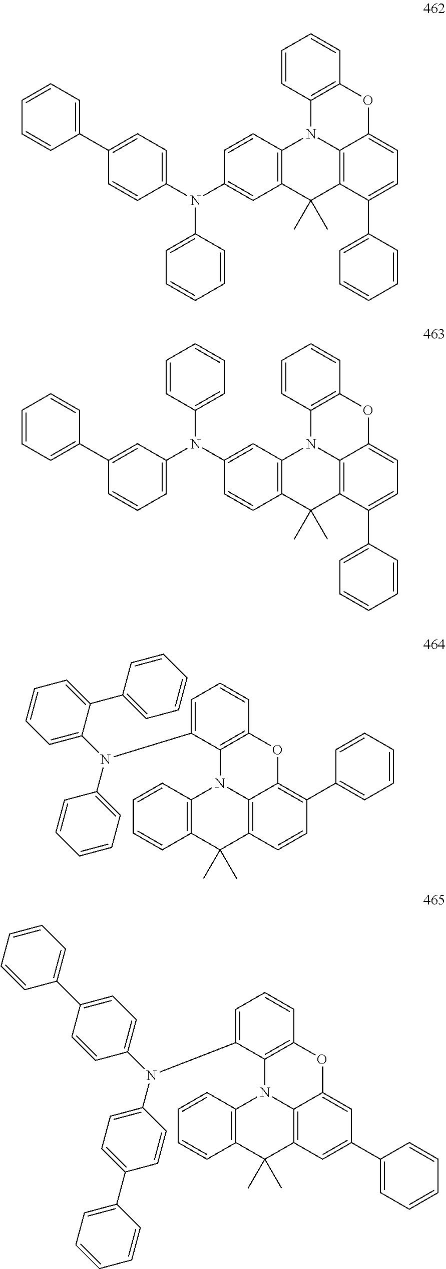

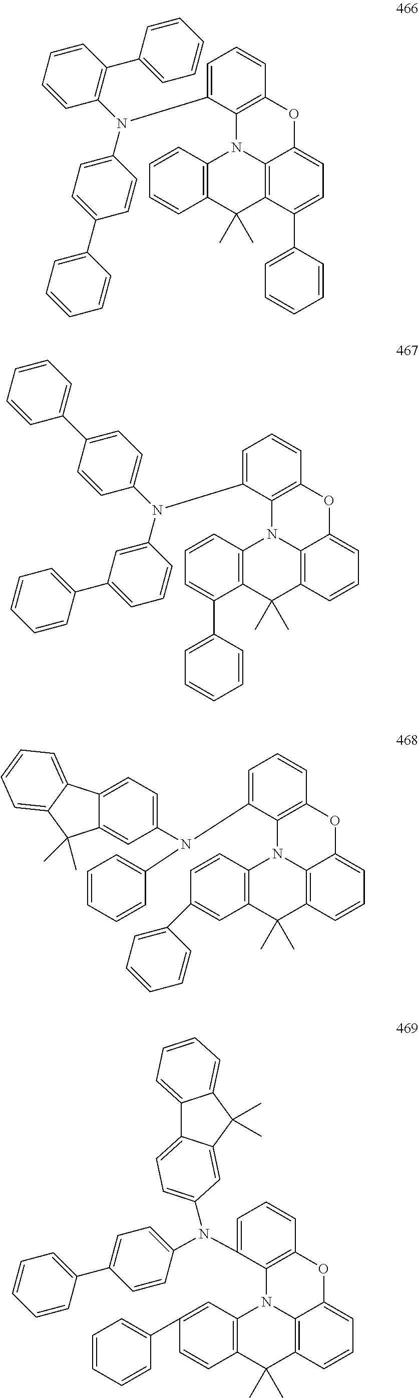 [SMILES: CC1(C)c2ccccc2N2c3c(cccc3N(c3ccc(-c4ccccc4)cc3)c3ccccc3-c3ccccc3)Oc3ccc(-c4ccccc4)c1c32 ; CC1(C)c2cccc3c2N(c2cccc(-c4ccccc4)c21)c1c(cccc1N(c1ccc(-c2ccccc2)cc1)c1cccc(-c2ccccc2)c1)O3 ; CC1(C)c2ccccc2-c2ccc(N(c3ccccc3)c3cccc4c3N3c5ccc(-c6ccccc6)cc5C(C)(C)c5cccc(c53)O4)cc21 ; CC1(C)c2ccccc2-c2ccc(N(c3ccc(-c4ccccc4)cc3)c3cccc4c3N3c5cc(-c6ccccc6)ccc5C(C)(C)c5cccc(c53)O4)cc21]

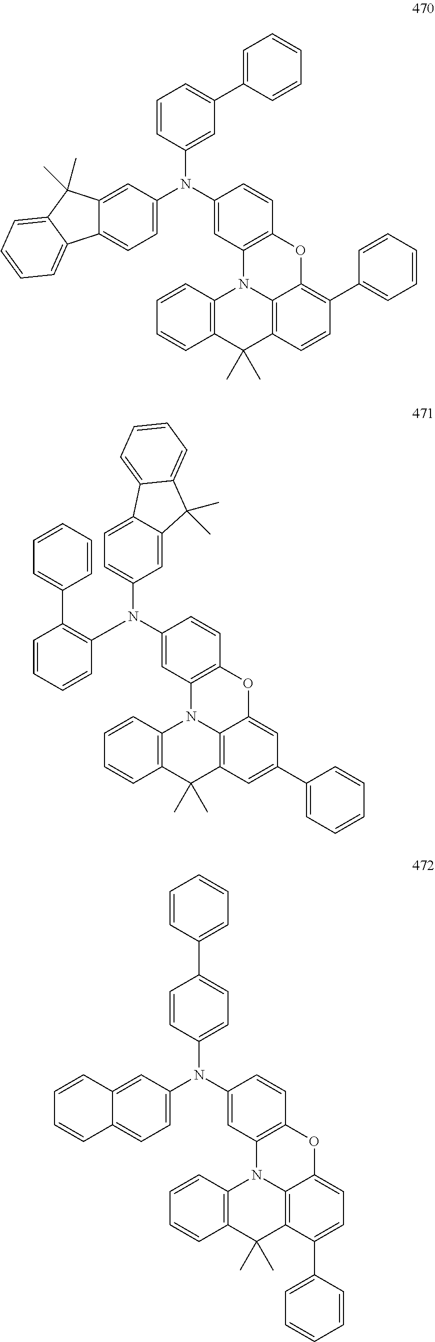 [SMILES: CC1(C)c2ccccc2-c2ccc(N(c3cccc(-c4ccccc4)c3)c3ccc4c(c3)N3c5ccccc5C(C)(C)c5ccc(-c6ccccc6)c(c53)O4)cc21 ; CC1(C)c2ccccc2-c2ccc(N(c3ccc4c(c3)N3c5ccccc5C(C)(C)c5cc(-c6ccccc6)cc(c53)O4)c3ccccc3-c3ccccc3)cc21 ; CC1(C)c2ccccc2N2c3cc(N(c4ccc(-c5ccccc5)cc4)c4ccc5ccccc5c4)ccc3Oc3ccc(-c4ccccc4)c1c32]

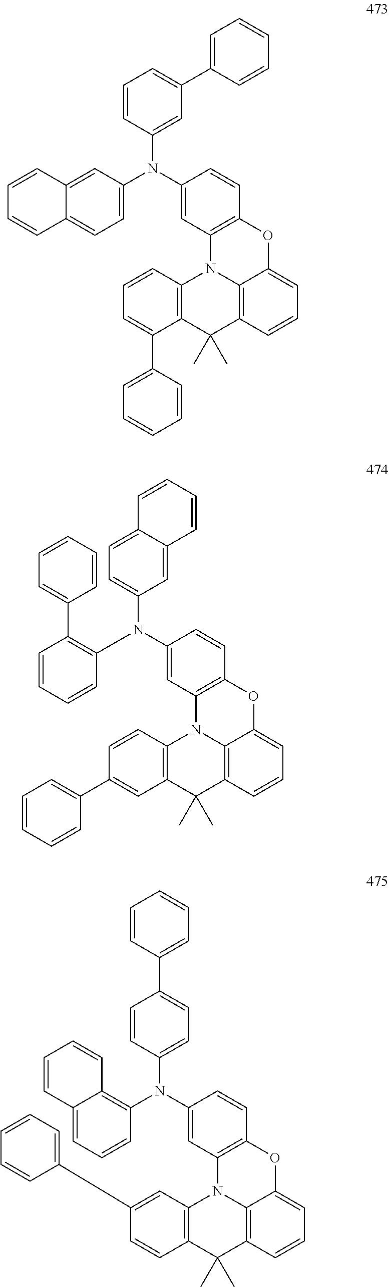

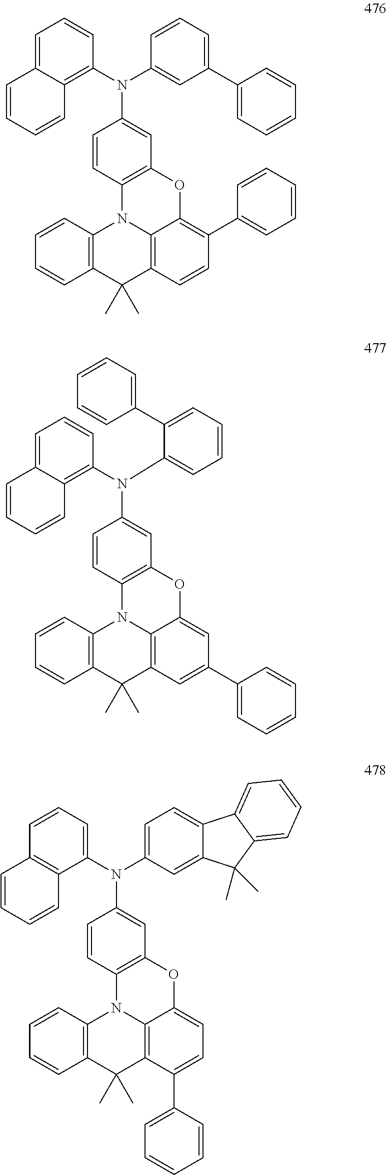

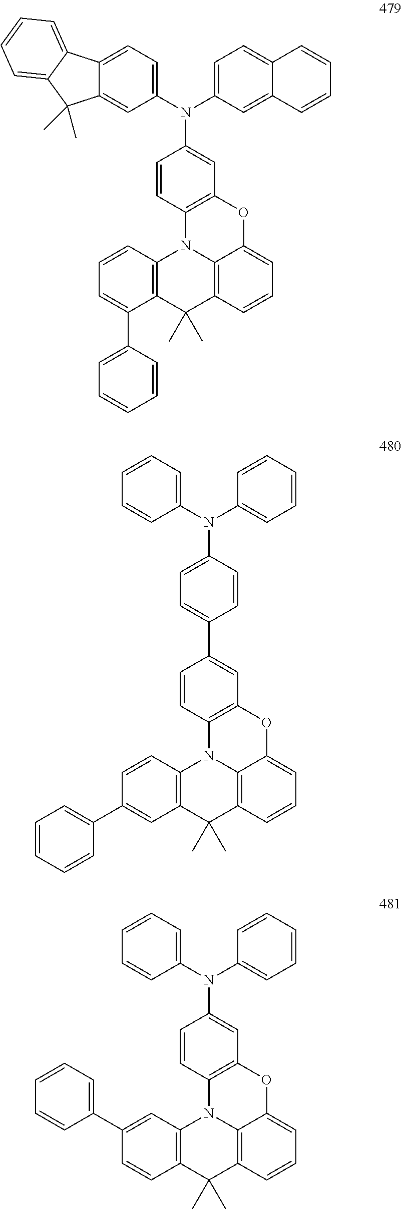 [SMILES: CC1(C)c2ccccc2-c2ccc(N(c3ccc4c(c3)Oc3cccc5c3N4c3cccc(-c4ccccc4)c3C5(C)C)c3ccc4ccccc4c3)cc21 ; CC1(C)c2cc(-c3ccccc3)ccc2N2c3ccc(-c4ccc(N(c5ccccc5)c5ccccc5)cc4)cc3Oc3cccc1c32 ; CC1(C)c2ccc(-c3ccccc3)cc2N2c3ccc(N(c4ccccc4)c4ccccc4)cc3Oc3cccc1c32]

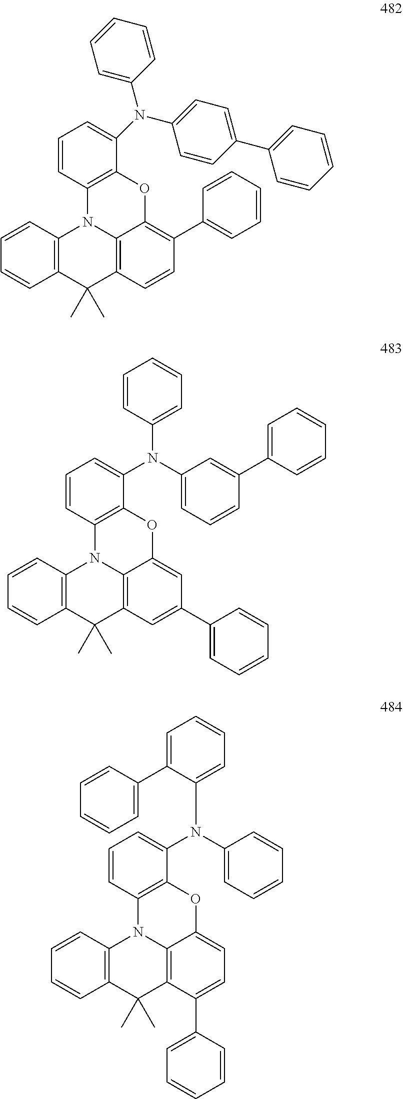 [SMILES: CC1(C)c2ccccc2N2c3cccc(N(c4ccccc4)c4ccc(-c5ccccc5)cc4)c3Oc3c(-c4ccccc4)ccc1c32 ; CC1(C)c2ccccc2N2c3cccc(N(c4ccccc4)c4cccc(-c5ccccc5)c4)c3Oc3cc(-c4ccccc4)cc1c32 ; CC1(C)c2ccccc2N2c3cccc(N(c4ccccc4)c4ccccc4-c4ccccc4)c3Oc3ccc(-c4ccccc4)c1c32]

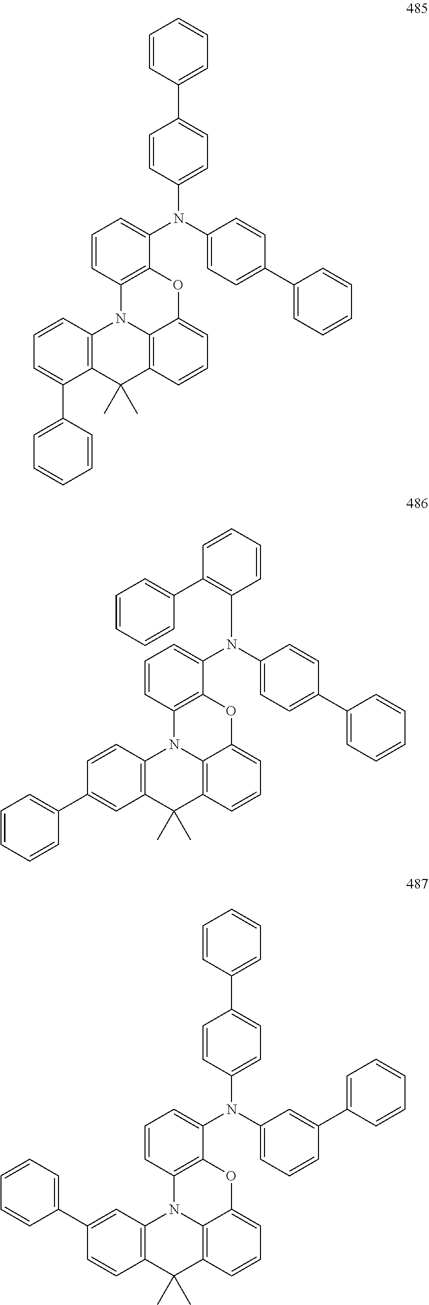 [SMILES: CC1(C)c2cccc3c2N(c2cccc(N(c4ccc(-c5ccccc5)cc4)c4ccc(-c5ccccc5)cc4)c2O3)c2cccc(-c3ccccc3)c21 ; CC1(C)c2cc(-c3ccccc3)ccc2N2c3cccc(N(c4ccc(-c5ccccc5)cc4)c4ccccc4-c4ccccc4)c3Oc3cccc1c32 ; CC1(C)c2ccc(-c3ccccc3)cc2N2c3cccc(N(c4ccc(-c5ccccc5)cc4)c4cccc(-c5ccccc5)c4)c3Oc3cccc1c32]

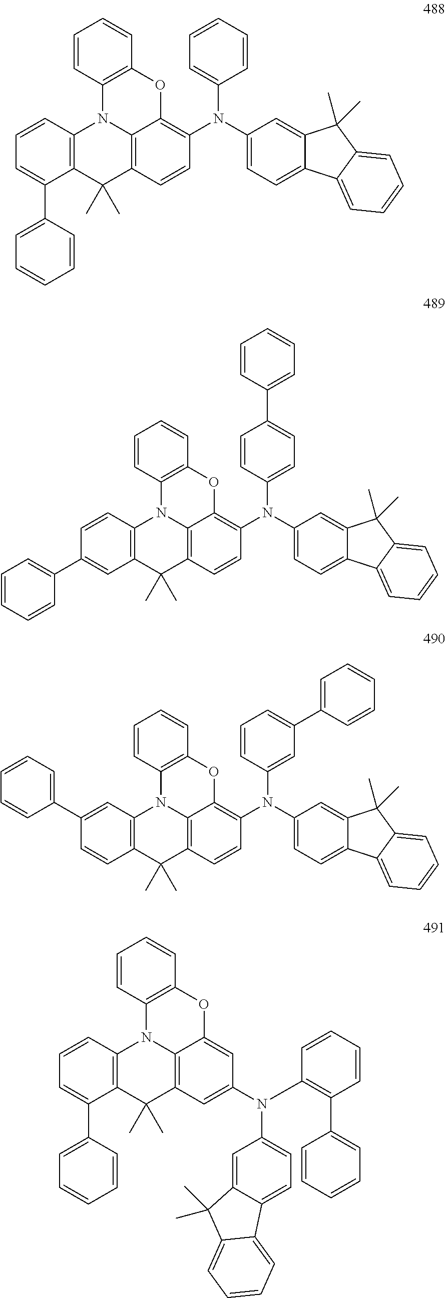 [SMILES: CC1(C)c2ccccc2-c2ccc(N(c3ccccc3)c3ccc4c5c3Oc3ccccc3N5c3cccc(-c5ccccc5)c3C4(C)C)cc21 ; CC1(C)c2ccccc2-c2ccc(N(c3ccc(-c4ccccc4)cc3)c3ccc4c5c3Oc3ccccc3N5c3ccc(-c5ccccc5)cc3C4(C)C)cc21 ; CC1(C)c2ccccc2-c2ccc(N(c3cccc(-c4ccccc4)c3)c3ccc4c5c3Oc3ccccc3N5c3cc(-c5ccccc5)ccc3C4(C)C)cc21 ; CC1(C)c2ccccc2-c2ccc(N(c3cc4c5c(c3)C(C)(C)c3c(-c6ccccc6)cccc3N5c3ccccc3O4)c3ccccc3-c3ccccc3)cc21]

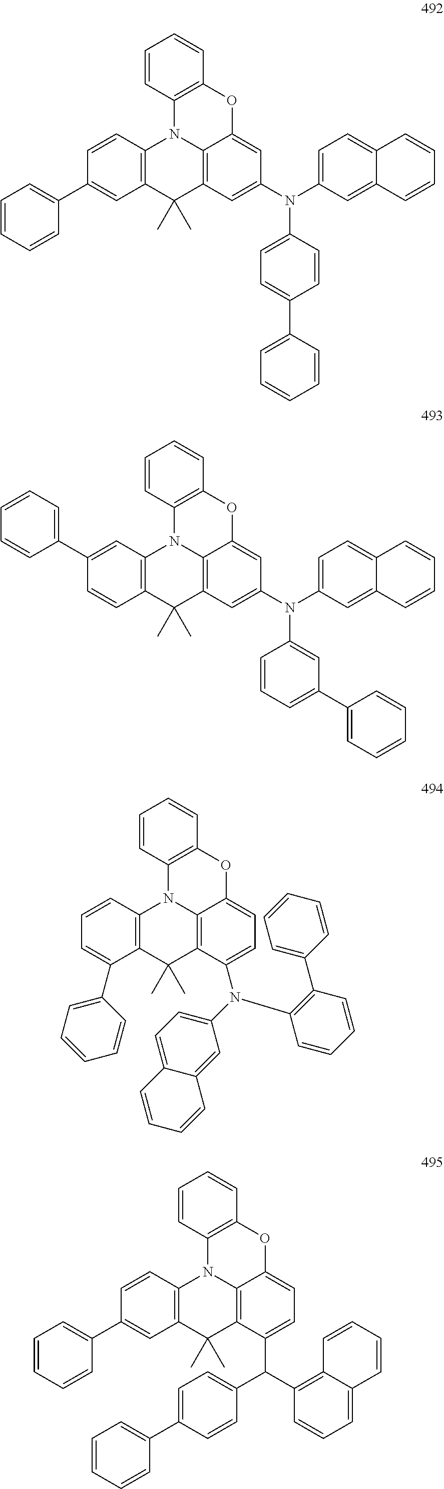 [SMILES: CC1(C)c2cc(-c3ccccc3)ccc2N2c3ccccc3Oc3cc(N(c4ccc(-c5ccccc5)cc4)c4ccc5ccccc5c4)cc1c32 ; CC1(C)c2ccc(-c3ccccc3)cc2N2c3ccccc3Oc3cc(N(c4cccc(-c5ccccc5)c4)c4ccc5ccccc5c4)cc1c32 ; CC1(C)c2c(-c3ccccc3)cccc2N2c3ccccc3Oc3ccc(N(c4ccc5ccccc5c4)c4ccccc4-c4ccccc4)c1c32 ; CC1(C)c2cc(-c3ccccc3)ccc2N2c3ccccc3Oc3ccc(C(c4ccc(-c5ccccc5)cc4)c4cccc5ccccc45)c1c32]

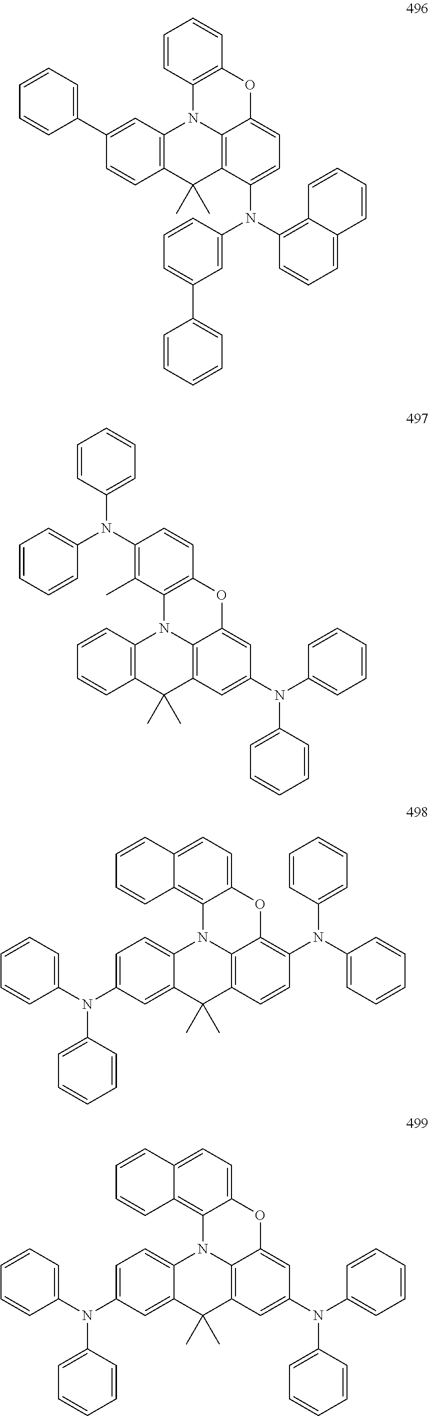 [SMILES: CC1(C)c2ccc(-c3ccccc3)cc2N2c3ccccc3Oc3ccc(N(c4cccc(-c5ccccc5)c4)c4cccc5ccccc45)c1c32 ; Cc1c(N(c2ccccc2)c2ccccc2)ccc2c1N1c3ccccc3C(C)(C)c3cc(N(c4ccccc4)c4ccccc4)cc(c31)O2 ; CC1(C)c2cc(N(c3ccccc3)c3ccccc3)ccc2N2c3c1ccc(N(c1ccccc1)c1ccccc1)c3Oc1ccc3ccccc3c12 ; CC1(C)c2cc(N(c3ccccc3)c3ccccc3)ccc2N2c3c(cc(N(c4ccccc4)c4ccccc4)cc31)Oc1ccc3ccccc3c12]

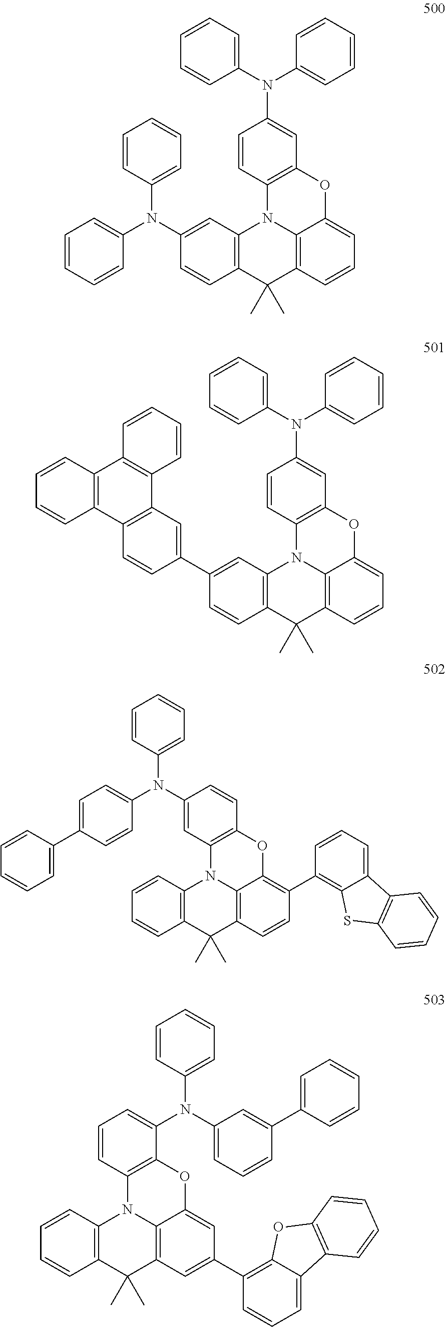

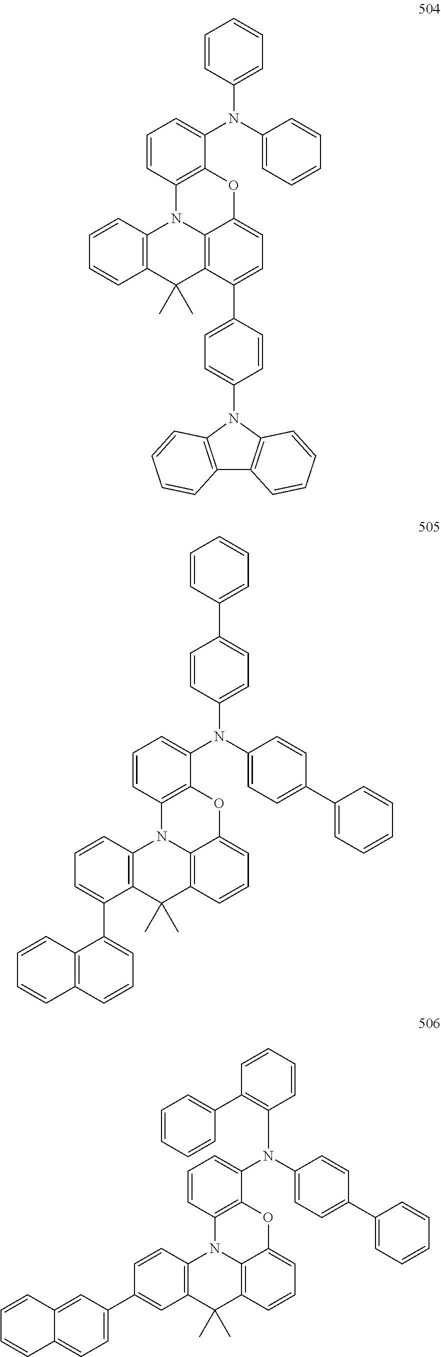 [SMILES: CC1(C)c2ccccc2N2c3cccc(N(c4ccccc4)c4ccccc4)c3Oc3ccc(-c4ccc(-n5c6ccccc6c6ccccc65)cc4)c1c32 ; CC1(C)c2cccc3c2N(c2cccc(N(c4ccc(-c5ccccc5)cc4)c4ccc(-c5ccccc5)cc4)c2O3)c2cccc(-c3cccc4ccccc34)c21 ; CC1(C)c2cc(-c3ccc4ccccc4c3)ccc2N2c3cccc(N(c4ccc(-c5ccccc5)cc4)c4ccccc4-c4ccccc4)c3Oc3cccc1c32]

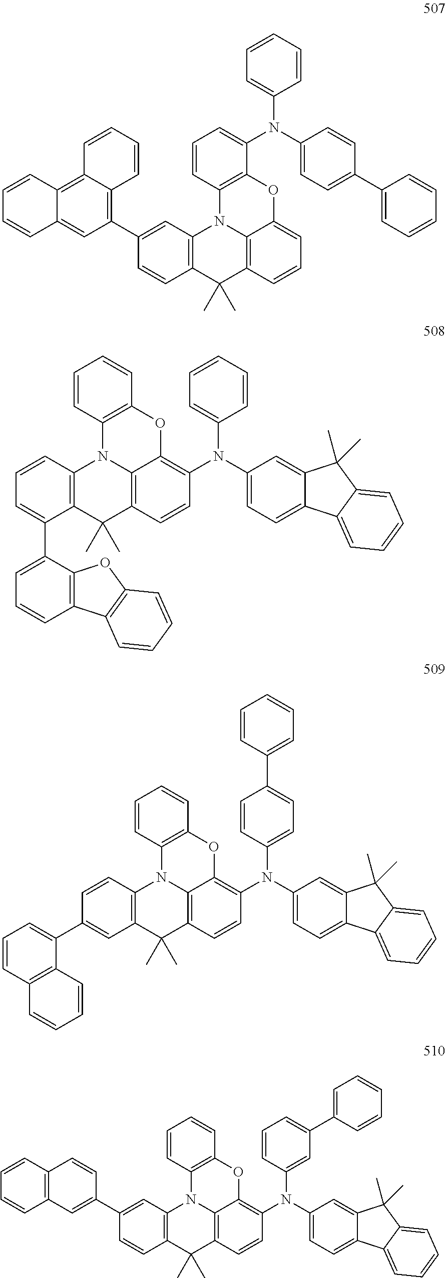 [SMILES: CC1(C)c2ccc(-c3cc4ccccc4c4ccccc34)cc2N2c3cccc(N(c4ccccc4)c4ccc(-c5ccccc5)cc4)c3Oc3cccc1c32 ; CC1(C)c2ccccc2-c2ccc(N(c3ccccc3)c3ccc4c5c3Oc3ccccc3N5c3cccc(-c5cccc6c5oc5ccccc56)c3C4(C)C)cc21 ; CC1(C)c2ccccc2-c2ccc(N(c3ccc(-c4ccccc4)cc3)c3ccc4c5c3Oc3ccccc3N5c3ccc(-c5cccc6ccccc56)cc3C4(C)C)cc21 ; CC1(C)c2ccccc2-c2ccc(N(c3cccc(-c4ccccc4)c3)c3ccc4c5c3Oc3ccccc3N5c3cc(-c5ccc6ccccc6c5)ccc3C4(C)C)cc21]

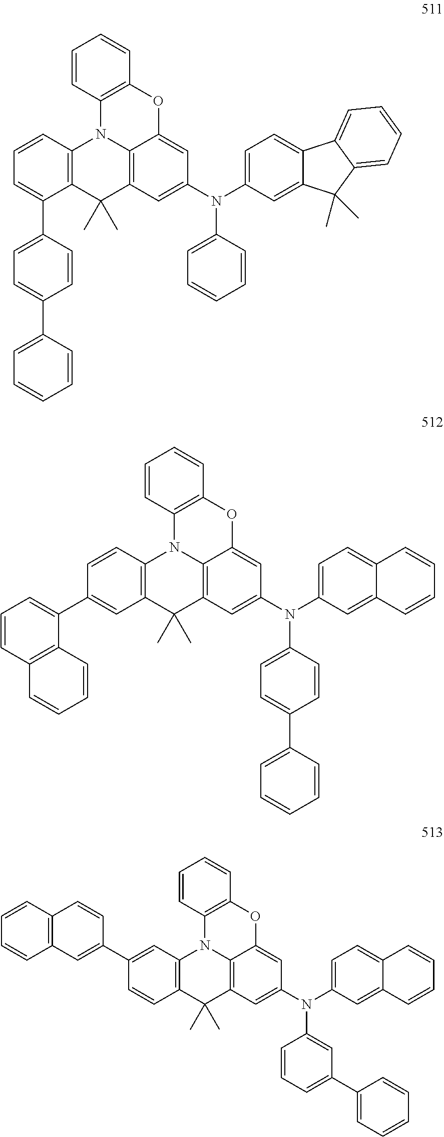 [SMILES: CC1(C)c2ccccc2-c2ccc(N(c3ccccc3)c3cc4c5c(c3)C(C)(C)c3c(-c6ccc(-c7ccccc7)cc6)cccc3N5c3ccccc3O4)cc21 ; CC1(C)c2cc(-c3cccc4ccccc34)ccc2N2c3ccccc3Oc3cc(N(c4ccc(-c5ccccc5)cc4)c4ccc5ccccc5c4)cc1c32 ; CC1(C)c2ccc(-c3ccc4ccccc4c3)cc2N2c3ccccc3Oc3cc(N(c4cccc(-c5ccccc5)c4)c4ccc5ccccc5c4)cc1c32]

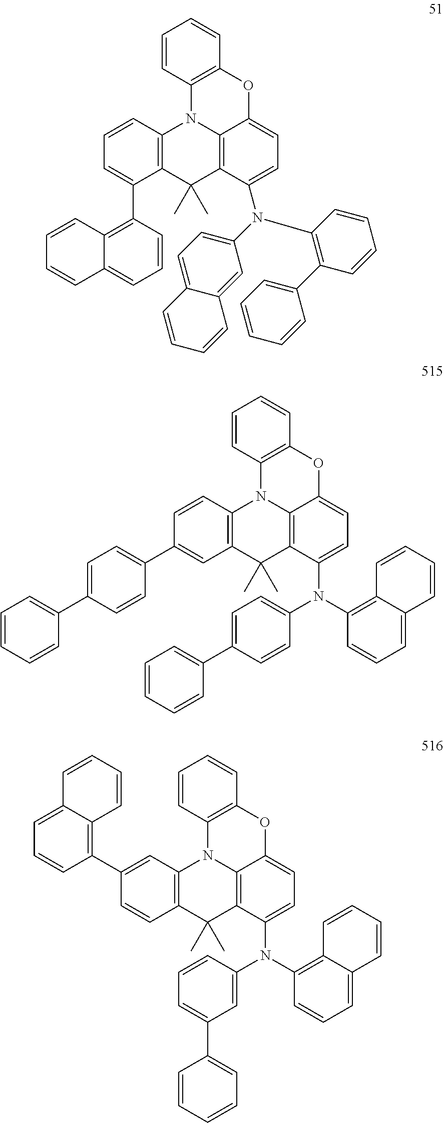 [SMILES: CC1(C)c2c(-c3cccc4ccccc34)cccc2N2c3ccccc3Oc3ccc(N(c4ccc5ccccc5c4)c4ccccc4-c4ccccc4)c1c32 ; CC1(C)c2cc(-c3ccc(-c4ccccc4)cc3)ccc2N2c3ccccc3Oc3ccc(N(c4ccc(-c5ccccc5)cc4)c4cccc5ccccc45)c1c32 ; CC1(C)c2ccc(-c3cccc4ccccc34)cc2N2c3ccccc3Oc3ccc(N(c4cccc(-c5ccccc5)c4)c4cccc5ccccc45)c1c32]

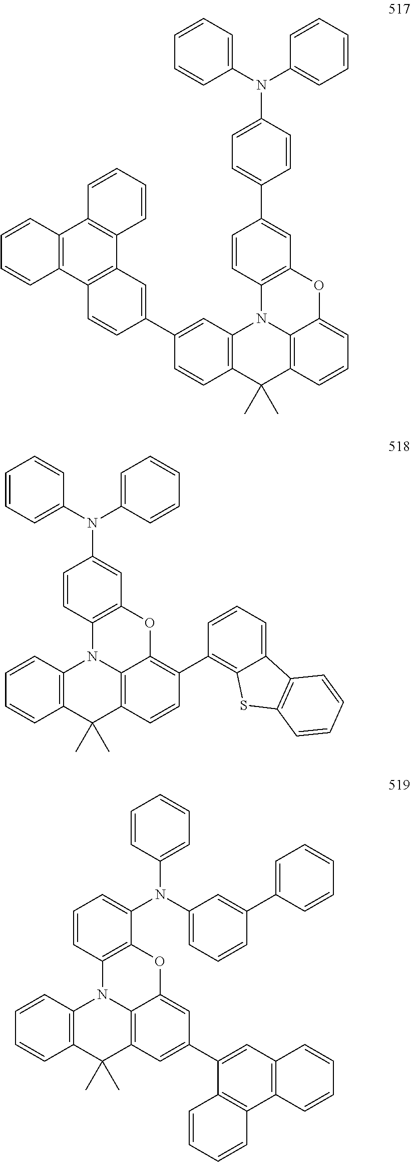 [SMILES: CC1(C)c2ccc(-c3ccc4c5ccccc5c5ccccc5c4c3)cc2N2c3ccc(-c4ccc(N(c5ccccc5)c5ccccc5)cc4)cc3Oc3cccc1c32 ; CC1(C)c2ccccc2N2c3ccc(N(c4ccccc4)c4ccccc4)cc3Oc3c(-c4cccc5c4sc4ccccc45)ccc1c32 ; CC1(C)c2ccccc2N2c3cccc(N(c4ccccc4)c4cccc(-c5ccccc5)c4)c3Oc3cc(-c4cc5ccccc5c5ccccc45)cc1c32]

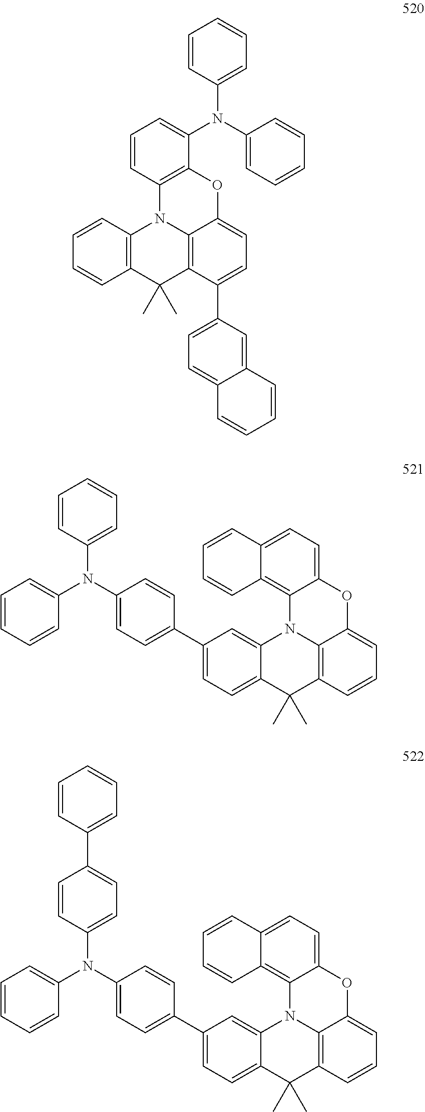 [SMILES: CC1(C)c2ccccc2N2c3cccc(N(c4ccccc4)c4ccccc4)c3Oc3ccc(-c4ccc5ccccc5c4)c1c32 ; CC1(C)c2ccc(-c3ccc(N(c4ccccc4)c4ccccc4)cc3)cc2N2c3c(cccc31)Oc1ccc3ccccc3c12 ; CC1(C)c2ccc(-c3ccc(N(c4ccccc4)c4ccc(-c5ccccc5)cc4)cc3)cc2N2c3c(cccc31)Oc1ccc3ccccc3c12]

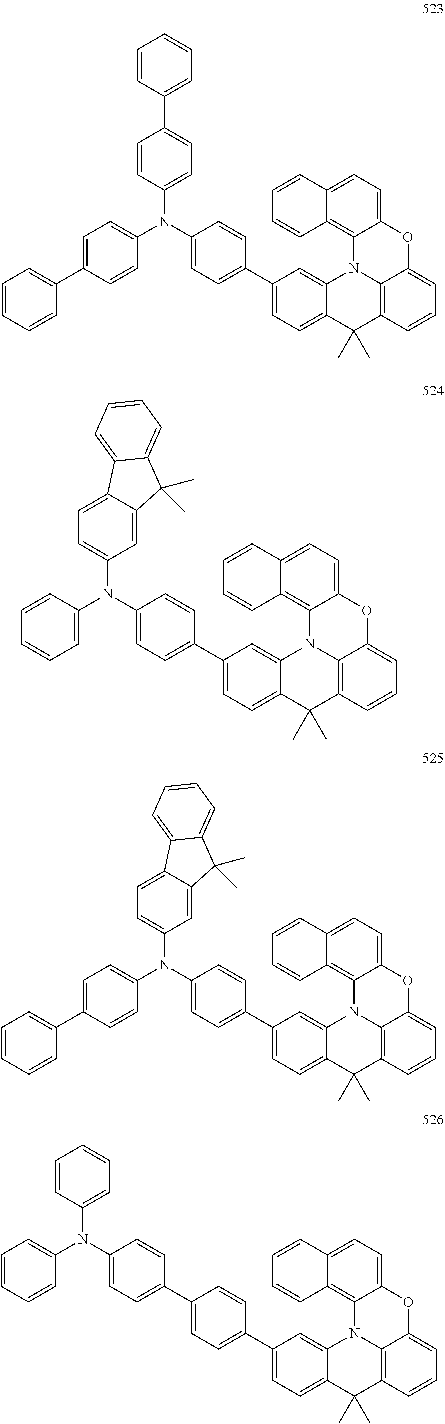 [SMILES: CC1(C)c2ccc(-c3ccc(N(c4ccc(-c5ccccc5)cc4)c4ccc(-c5ccccc5)cc4)cc3)cc2N2c3c(cccc31)Oc1ccc3ccccc3c12 ; CC1(C)c2ccccc2-c2ccc(N(c3ccccc3)c3ccc(-c4ccc5c(c4)N4c6c(cccc6C5(C)C)Oc5ccc6ccccc6c54)cc3)cc21 ; CC1(C)c2ccccc2-c2ccc(N(c3ccc(-c4ccccc4)cc3)c3ccc(-c4ccc5c(c4)N4c6c(cccc6C5(C)C)Oc5ccc6ccccc6c54)cc3)cc21 ; CC1(C)c2ccc(-c3ccc(-c4ccc(N(c5ccccc5)c5ccccc5)cc4)cc3)cc2N2c3c(cccc31)Oc1ccc3ccccc3c12]

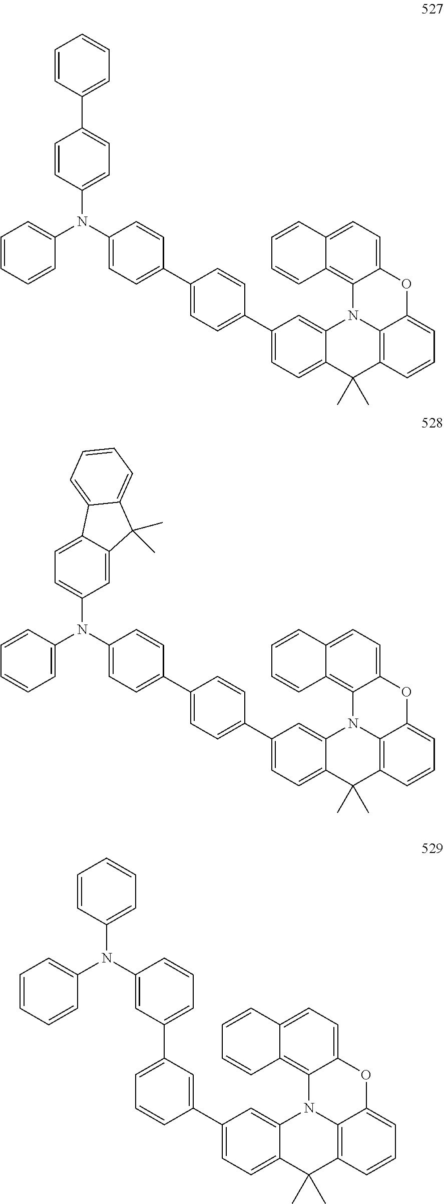 [SMILES: CC1(C)c2ccc(-c3ccc(-c4ccc(N(c5ccccc5)c5ccc(-c6ccccc6)cc5)cc4)cc3)cc2N2c3c(cccc31)Oc1ccc3ccccc3c12 ; CC1(C)c2ccccc2-c2ccc(N(c3ccccc3)c3ccc(-c4ccc(-c5ccc6c(c5)N5c7c(cccc7C6(C)C)Oc6ccc7ccccc7c65)cc4)cc3)cc21 ; CC1(C)c2ccc(-c3cccc(-c4cccc(N(c5ccccc5)c5ccccc5)c4)c3)cc2N2c3c(cccc31)Oc1ccc3ccccc3c12]

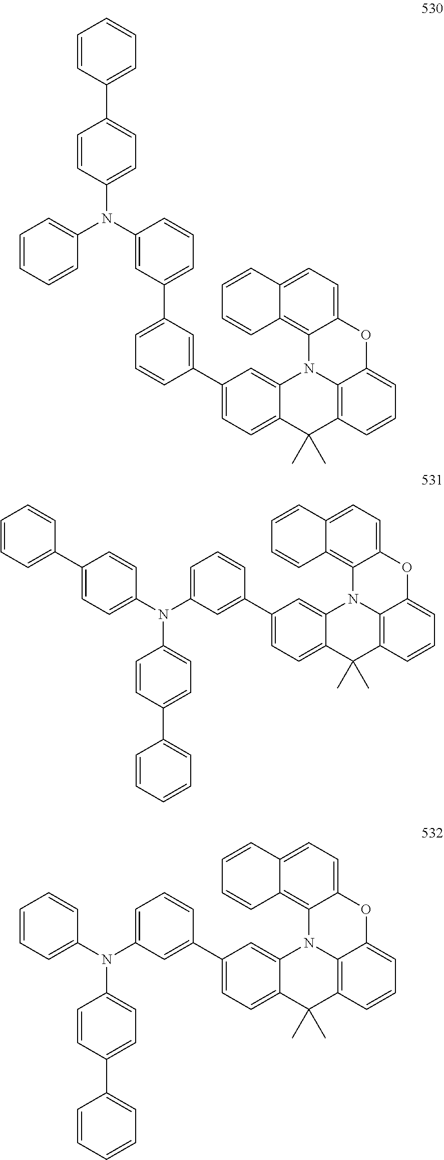

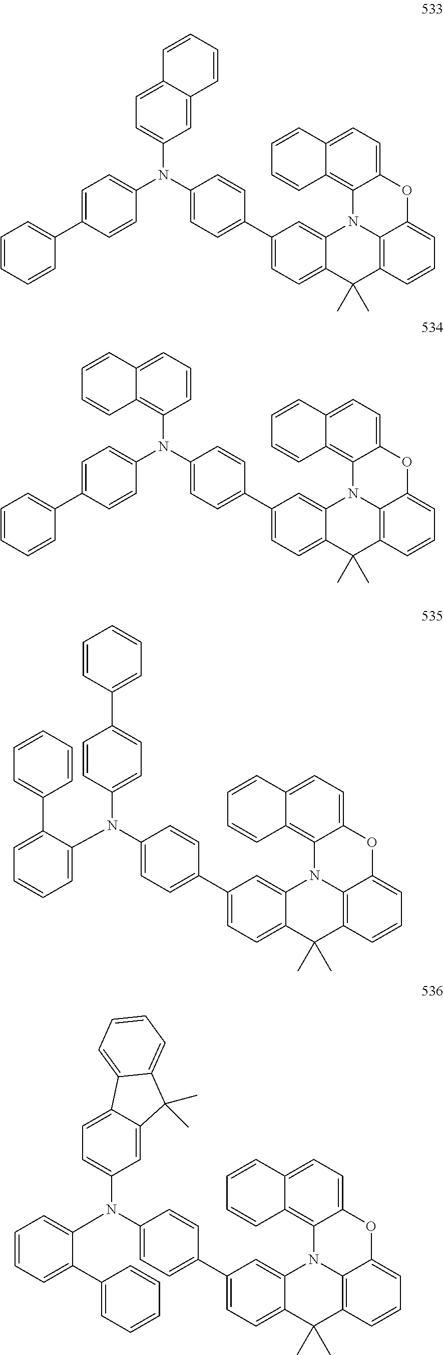 [SMILES: CC1(C)c2ccc(-c3ccc(N(c4ccc(-c5ccccc5)cc4)c4ccc5ccccc5c4)cc3)cc2N2c3c(cccc31)Oc1ccc3ccccc3c12 ; CC1(C)c2ccc(-c3ccc(N(c4ccc(-c5ccccc5)cc4)c4cccc5ccccc45)cc3)cc2N2c3c(cccc31)Oc1ccc3ccccc3c12 ; CC1(C)c2ccc(-c3ccc(N(c4ccc(-c5ccccc5)cc4)c4ccccc4-c4ccccc4)cc3)cc2N2c3c(cccc31)Oc1ccc3ccccc3c12 ; CC1(C)c2ccccc2-c2ccc(N(c3ccc(-c4ccc5c(c4)N4c6c(cccc6C5(C)C)Oc5ccc6ccccc6c54)cc3)c3ccccc3-c3ccccc3)cc21]

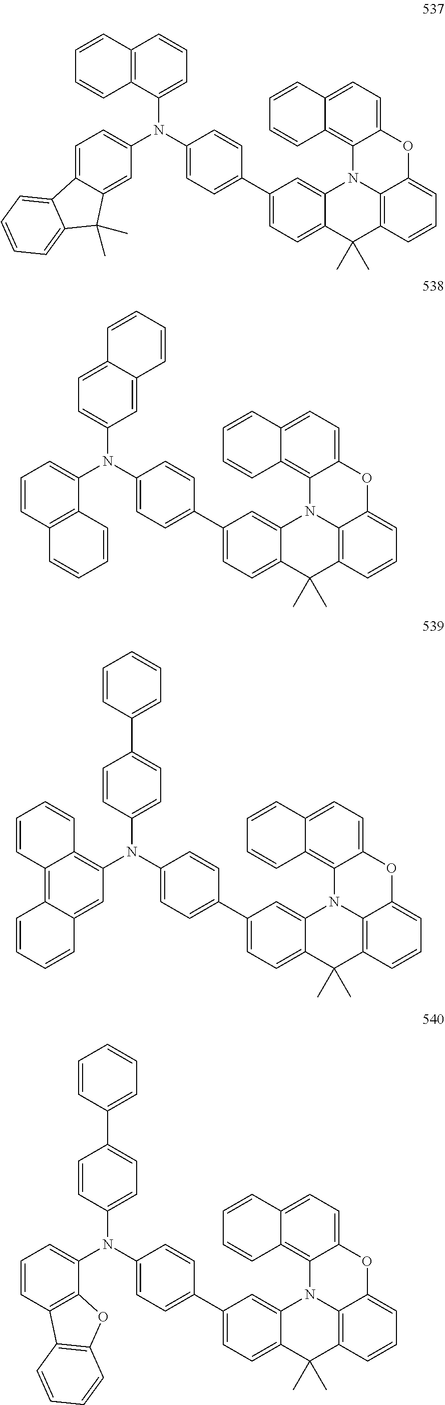 [SMILES: CC1(C)c2ccccc2-c2ccc(N(c3ccc(-c4ccc5c(c4)N4c6c(cccc6C5(C)C)Oc5ccc6ccccc6c54)cc3)c3cccc4ccccc34)cc21 ; CC1(C)c2ccc(-c3ccc(N(c4ccc5ccccc5c4)c4cccc5ccccc45)cc3)cc2N2c3c(cccc31)Oc1ccc3ccccc3c12 ; CC1(C)c2ccc(-c3ccc(N(c4ccc(-c5ccccc5)cc4)c4cc5ccccc5c5ccccc45)cc3)cc2N2c3c(cccc31)Oc1ccc3ccccc3c12 ; CC1(C)c2ccc(-c3ccc(N(c4ccc(-c5ccccc5)cc4)c4cccc5c4oc4ccccc45)cc3)cc2N2c3c(cccc31)Oc1ccc3ccccc3c12]

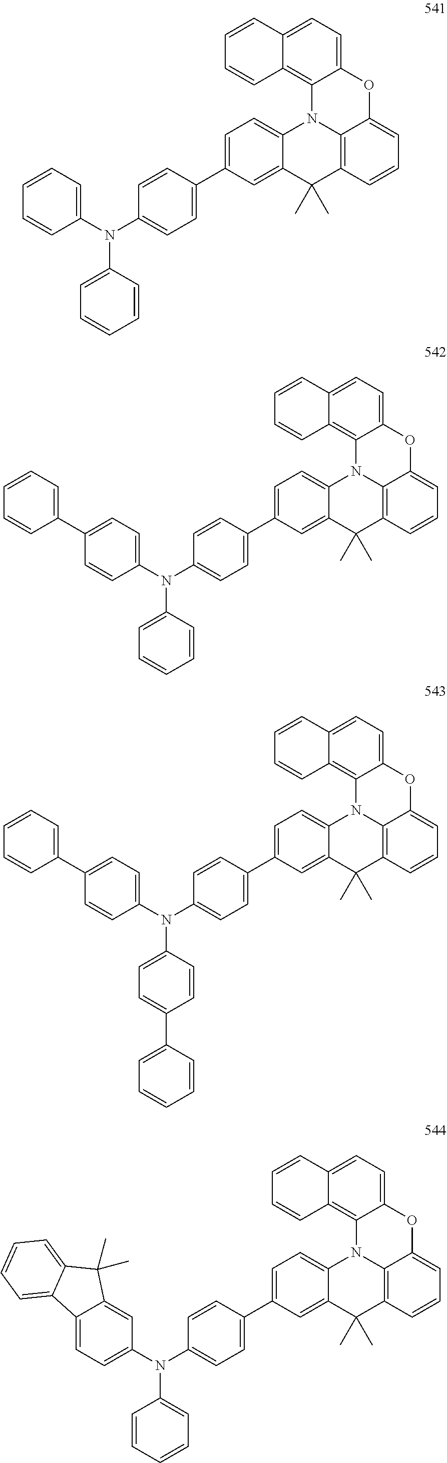 [SMILES: CC1(C)c2cc(-c3ccc(N(c4ccccc4)c4ccccc4)cc3)ccc2N2c3c(cccc31)Oc1ccc3ccccc3c12 ; CC1(C)c2cc(-c3ccc(N(c4ccccc4)c4ccc(-c5ccccc5)cc4)cc3)ccc2N2c3c(cccc31)Oc1ccc3ccccc3c12 ; CC1(C)c2cc(-c3ccc(N(c4ccc(-c5ccccc5)cc4)c4ccc(-c5ccccc5)cc4)cc3)ccc2N2c3c(cccc31)Oc1ccc3ccccc3c12 ; CC1(C)c2ccccc2-c2ccc(N(c3ccccc3)c3ccc(-c4ccc5c(c4)C(C)(C)c4cccc6c4N5c4c(ccc5ccccc45)O6)cc3)cc21]

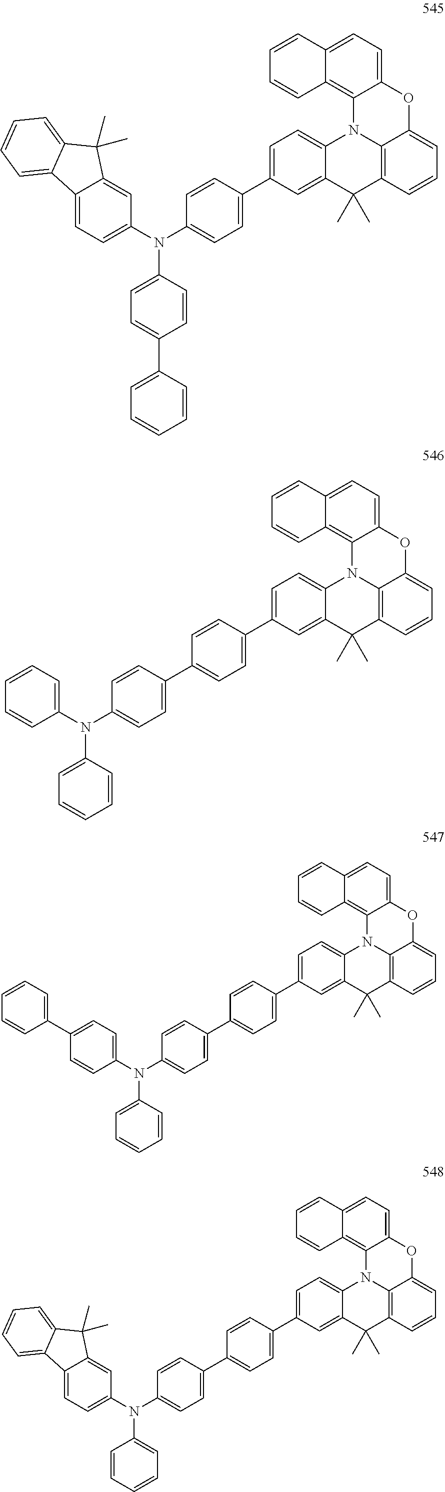 [SMILES: CC1(C)c2ccccc2-c2ccc(N(c3ccc(-c4ccccc4)cc3)c3ccc(-c4ccc5c(c4)C(C)(C)c4cccc6c4N5c4c(ccc5ccccc45)O6)cc3)cc21 ; CC1(C)c2cc(-c3ccc(-c4ccc(N(c5ccccc5)c5ccccc5)cc4)cc3)ccc2N2c3c(cccc31)Oc1ccc3ccccc3c12 ; CC1(C)c2cc(-c3ccc(-c4ccc(N(c5ccccc5)c5ccc(-c6ccccc6)cc5)cc4)cc3)ccc2N2c3c(cccc31)Oc1ccc3ccccc3c12 ; CC1(C)c2ccccc2-c2ccc(N(c3ccccc3)c3ccc(-c4ccc(-c5ccc6c(c5)C(C)(C)c5cccc7c5N6c5c(ccc6ccccc56)O7)cc4)cc3)cc21]

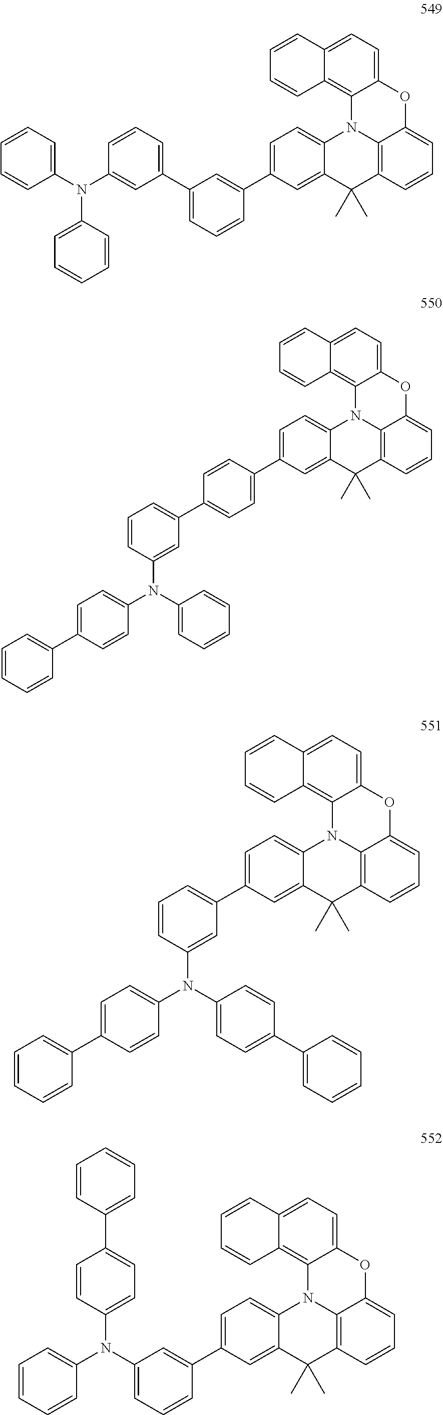 [SMILES: CC1(C)c2cc(-c3cccc(-c4cccc(N(c5ccccc5)c5ccccc5)c4)c3)ccc2N2c3c(cccc31)Oc1ccc3ccccc3c12 ; CC1(C)c2cc(-c3ccc(-c4cccc(N(c5ccccc5)c5ccc(-c6ccccc6)cc5)c4)cc3)ccc2N2c3c(cccc31)Oc1ccc3ccccc3c12 ; CC1(C)c2cc(-c3cccc(N(c4ccc(-c5ccccc5)cc4)c4ccc(-c5ccccc5)cc4)c3)ccc2N2c3c(cccc31)Oc1ccc3ccccc3c12 ; CC1(C)c2cc(-c3cccc(N(c4ccccc4)c4ccc(-c5ccccc5)cc4)c3)ccc2N2c3c(cccc31)Oc1ccc3ccccc3c12]

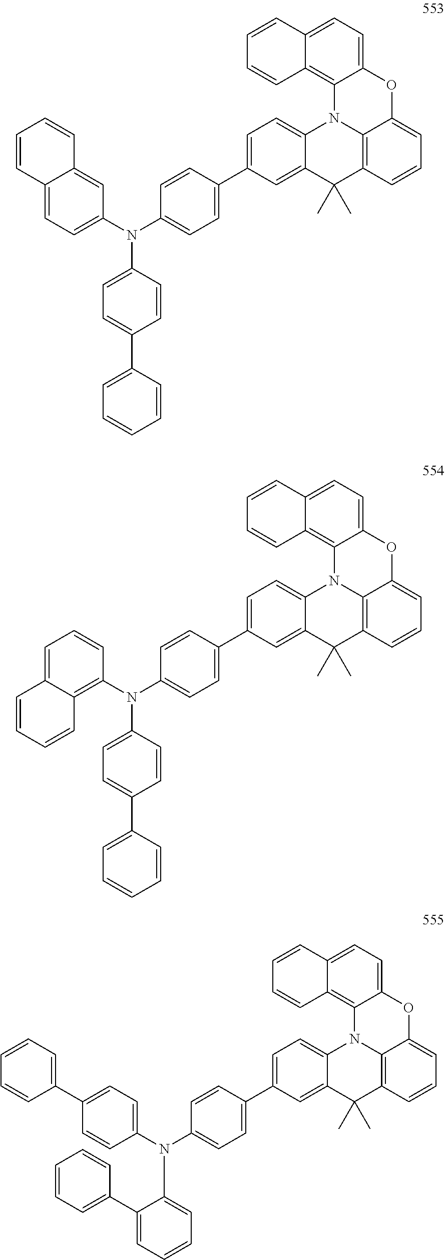 [SMILES: CC1(C)c2cc(-c3ccc(N(c4ccc(-c5ccccc5)cc4)c4ccc5ccccc5c4)cc3)ccc2N2c3c(cccc31)Oc1ccc3ccccc3c12 ; CC1(C)c2cc(-c3ccc(N(c4ccc(-c5ccccc5)cc4)c4cccc5ccccc45)cc3)ccc2N2c3c(cccc31)Oc1ccc3ccccc3c12 ; CC1(C)c2cc(-c3ccc(N(c4ccc(-c5ccccc5)cc4)c4ccccc4-c4ccccc4)cc3)ccc2N2c3c(cccc31)Oc1ccc3ccccc3c12]

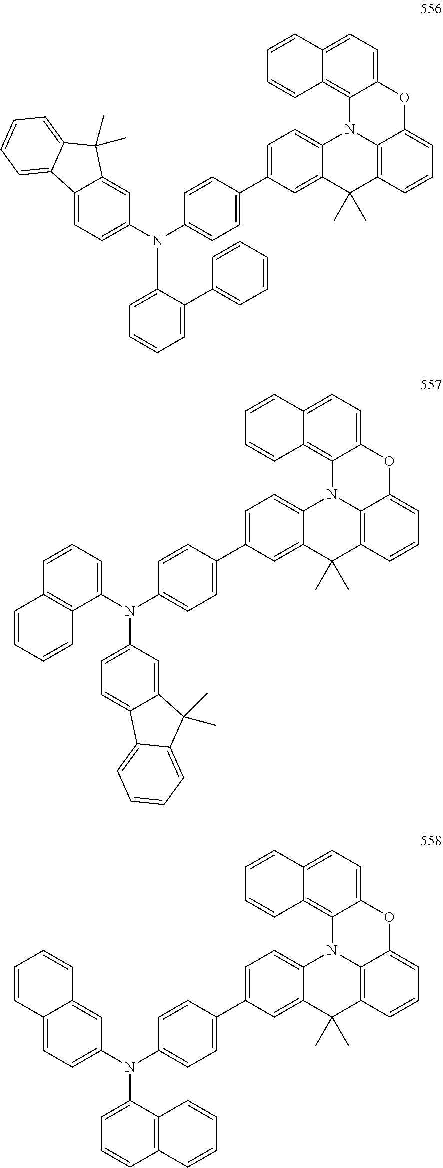 [SMILES: CC1(C)c2ccccc2-c2ccc(N(c3ccc(-c4ccc5c(c4)C(C)(C)c4cccc6c4N5c4c(ccc5ccccc45)O6)cc3)c3ccccc3-c3ccccc3)cc21 ; CC1(C)c2ccccc2-c2ccc(N(c3ccc(-c4ccc5c(c4)C(C)(C)c4cccc6c4N5c4c(ccc5ccccc45)O6)cc3)c3cccc4ccccc34)cc21 ; CC1(C)c2cc(-c3ccc(N(c4ccc5ccccc5c4)c4cccc5ccccc45)cc3)ccc2N2c3c(cccc31)Oc1ccc3ccccc3c12]

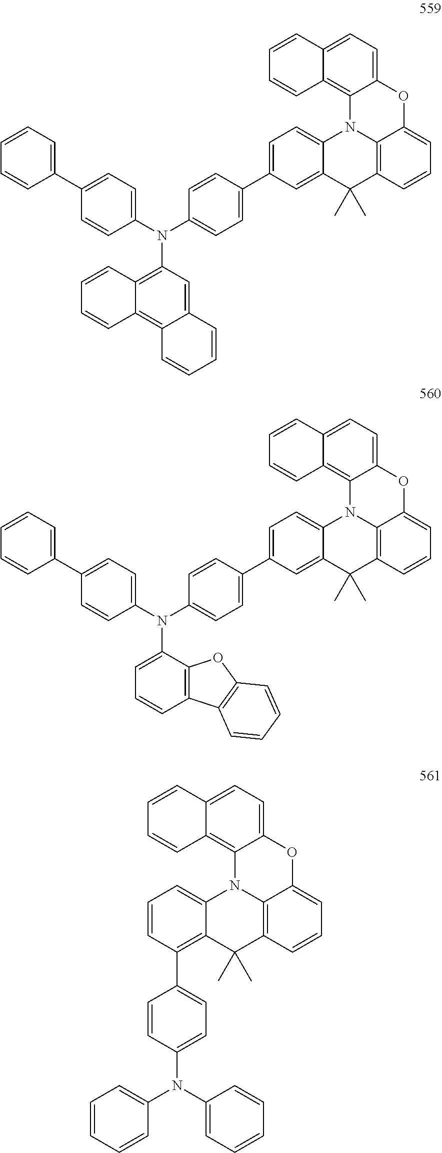 [SMILES: CC1(C)c2cc(-c3ccc(N(c4ccc(-c5ccccc5)cc4)c4cc5ccccc5c5ccccc45)cc3)ccc2N2c3c(cccc31)Oc1ccc3ccccc3c12 ; CC1(C)c2cc(-c3ccc(N(c4ccc(-c5ccccc5)cc4)c4cccc5c4oc4ccccc45)cc3)ccc2N2c3c(cccc31)Oc1ccc3ccccc3c12 ; CC1(C)c2cccc3c2N(c2cccc(-c4ccc(N(c5ccccc5)c5ccccc5)cc4)c21)c1c(ccc2ccccc12)O3]

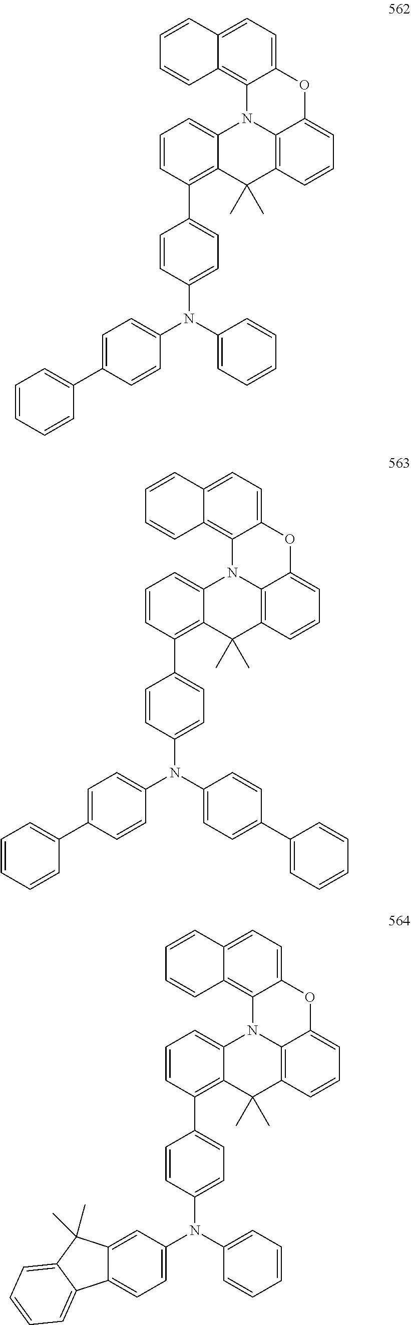 [SMILES: CC1(C)c2cccc3c2N(c2cccc(-c4ccc(N(c5ccccc5)c5ccc(-c6ccccc6)cc5)cc4)c21)c1c(ccc2ccccc12)O3 ; CC1(C)c2cccc3c2N(c2cccc(-c4ccc(N(c5ccc(-c6ccccc6)cc5)c5ccc(-c6ccccc6)cc5)cc4)c21)c1c(ccc2ccccc12)O3 ; CC1(C)c2ccccc2-c2ccc(N(c3ccccc3)c3ccc(-c4cccc5c4C(C)(C)c4cccc6c4N5c4c(ccc5ccccc45)O6)cc3)cc21]

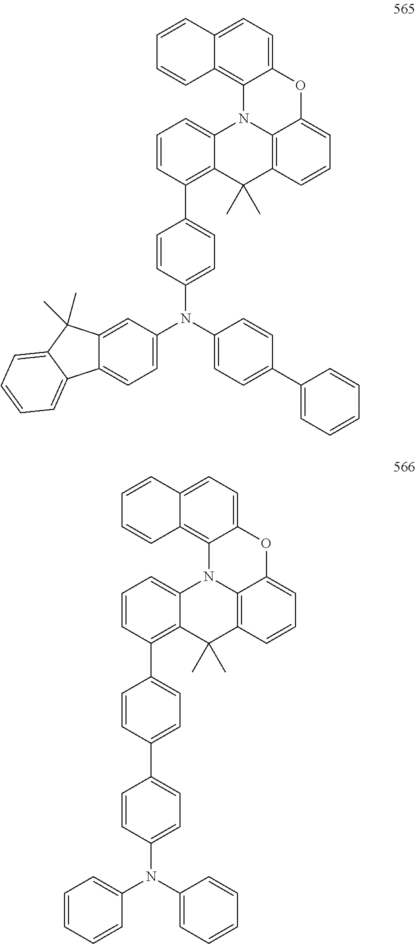 [SMILES: CC1(C)c2ccccc2-c2ccc(N(c3ccc(-c4ccccc4)cc3)c3ccc(-c4cccc5c4C(C)(C)c4cccc6c4N5c4c(ccc5ccccc45)O6)cc3)cc21 ; CC1(C)c2cccc3c2N(c2cccc(-c4ccc(-c5ccc(N(c6ccccc6)c6ccccc6)cc5)cc4)c21)c1c(ccc2ccccc12)O3]

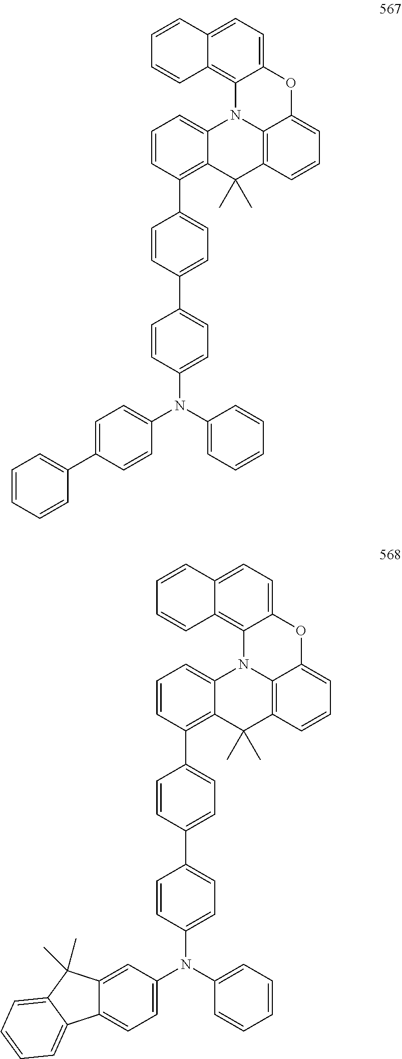 [SMILES: CC1(C)c2cccc3c2N(c2cccc(-c4ccc(-c5ccc(N(c6ccccc6)c6ccc(-c7ccccc7)cc6)cc5)cc4)c21)c1c(ccc2ccccc12)O3 ; CC1(C)c2ccccc2-c2ccc(N(c3ccccc3)c3ccc(-c4ccc(-c5cccc6c5C(C)(C)c5cccc7c5N6c5c(ccc6ccccc56)O7)cc4)cc3)cc21]

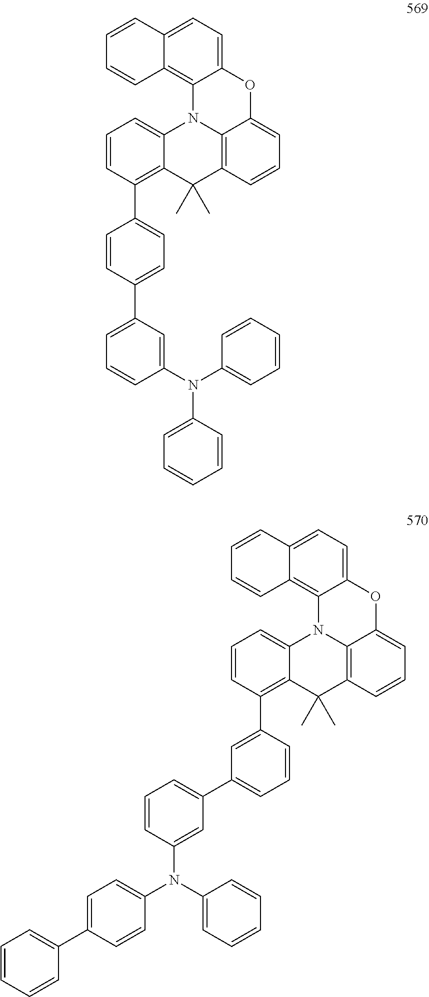 [SMILES: CC1(C)c2cccc3c2N(c2cccc(-c4ccc(-c5cccc(N(c6ccccc6)c6ccccc6)c5)cc4)c21)c1c(ccc2ccccc12)O3 ; CC1(C)c2cccc3c2N(c2cccc(-c4cccc(-c5cccc(N(c6ccccc6)c6ccc(-c7ccccc7)cc6)c5)c4)c21)c1c(ccc2ccccc12)O3]

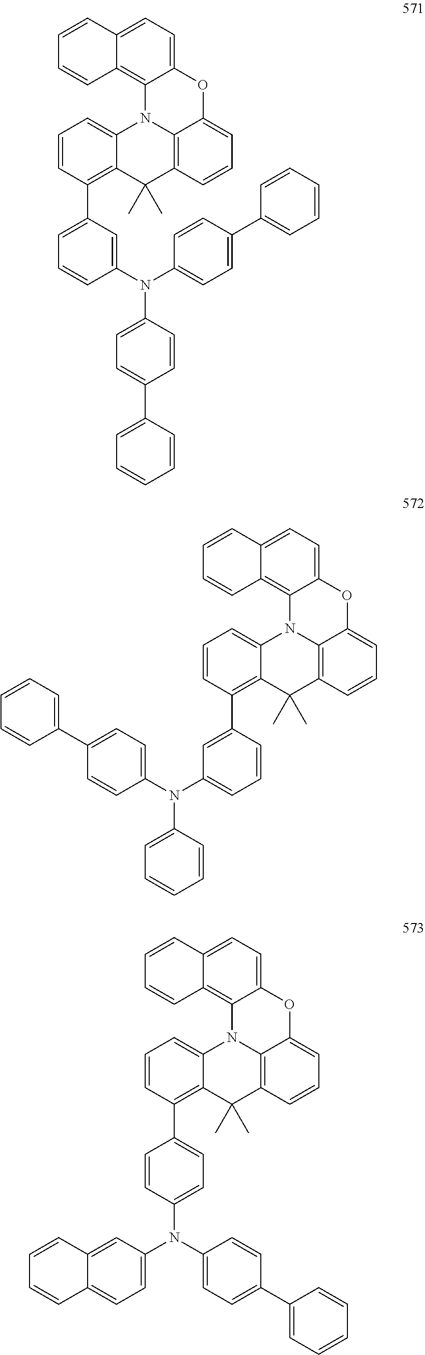

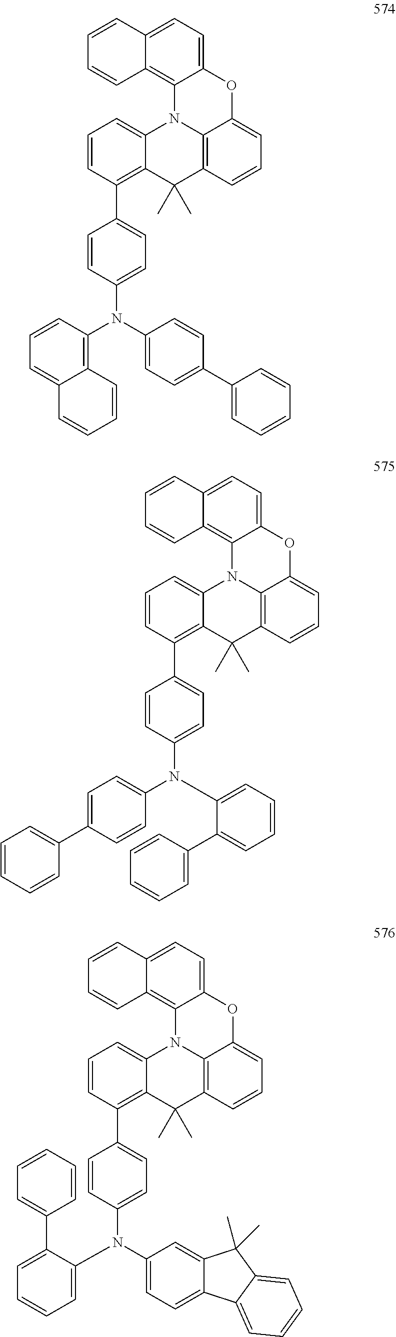

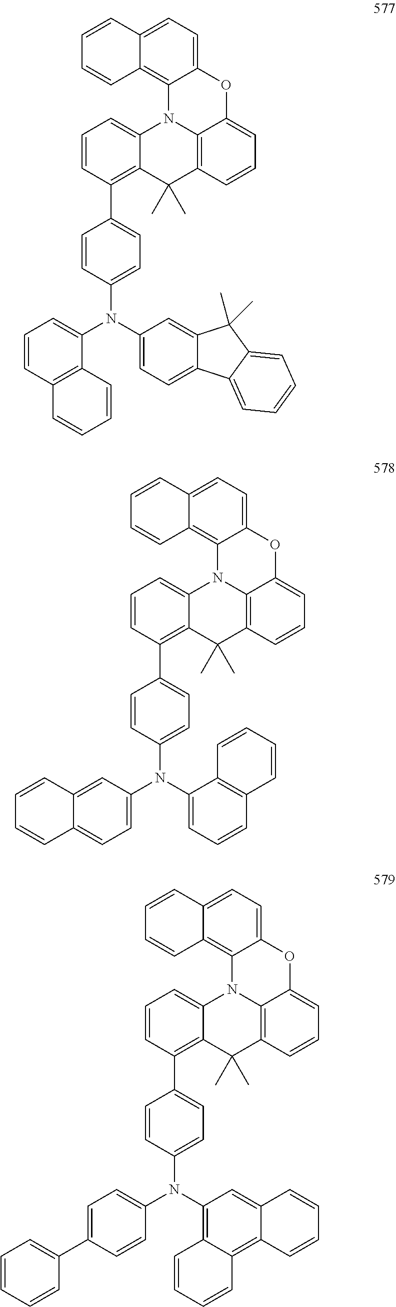 [SMILES: CC1(C)c2ccccc2-c2ccc(N(c3ccc(-c4cccc5c4C(C)(C)c4cccc6c4N5c4c(ccc5ccccc45)O6)cc3)c3cccc4ccccc34)cc21 ; CC1(C)c2cccc3c2N(c2cccc(-c4ccc(N(c5ccc6ccccc6c5)c5cccc6ccccc56)cc4)c21)c1c(ccc2ccccc12)O3 ; CC1(C)c2cccc3c2N(c2cccc(-c4ccc(N(c5ccc(-c6ccccc6)cc5)c5cc6ccccc6c6ccccc56)cc4)c21)c1c(ccc2ccccc12)O3]

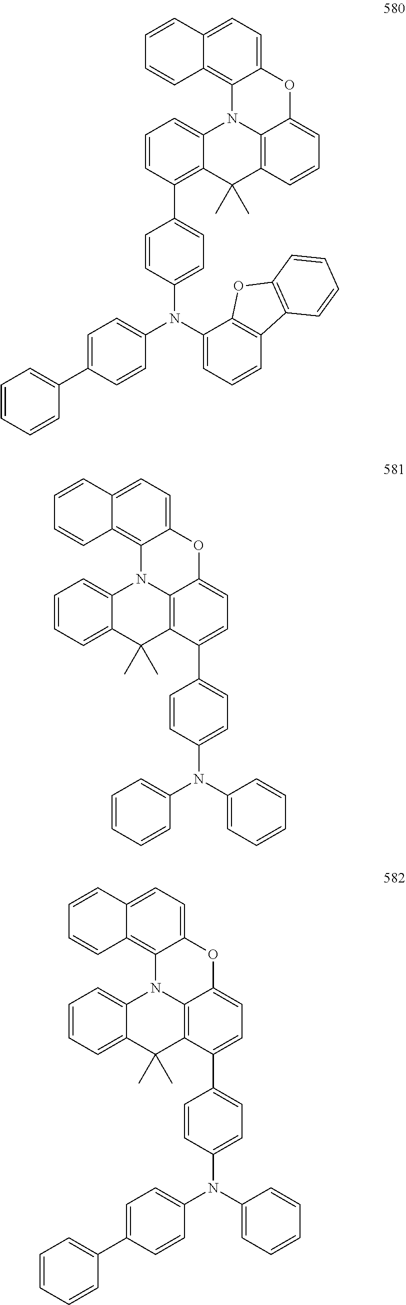

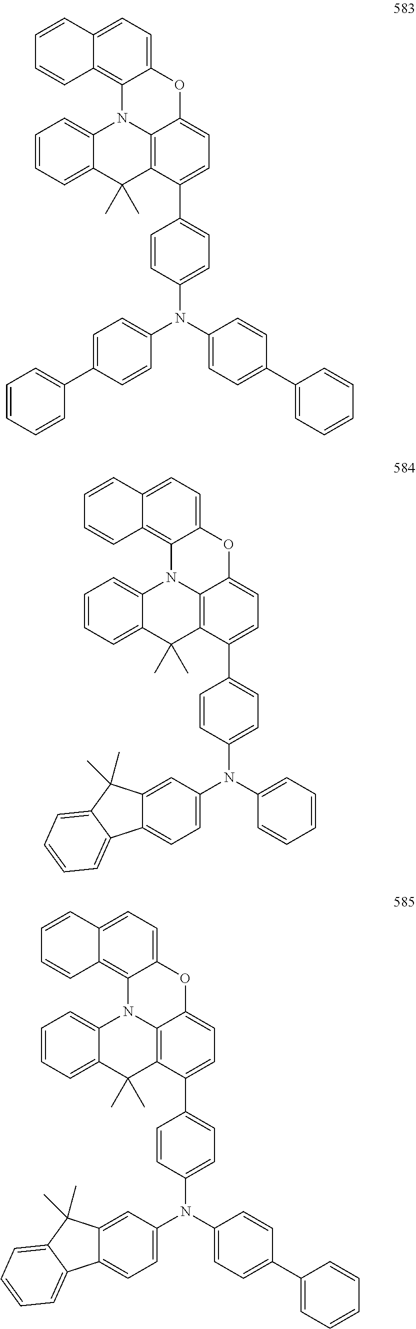

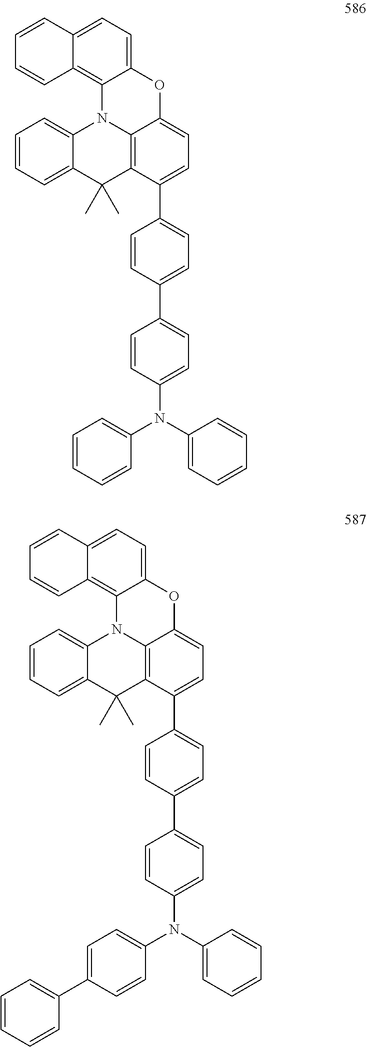

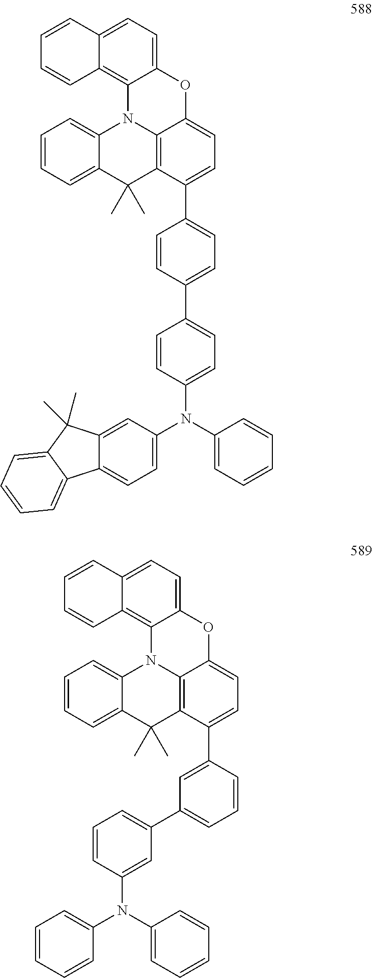 [SMILES: CC1(C)c2ccccc2-c2ccc(N(c3ccccc3)c3ccc(-c4ccc(-c5ccc6c7c5C(C)(C)c5ccccc5N7c5c(ccc7ccccc57)O6)cc4)cc3)cc21 ; CC1(C)c2ccccc2N2c3c(ccc(-c4cccc(-c5cccc(N(c6ccccc6)c6ccccc6)c5)c4)c31)Oc1ccc3ccccc3c12]

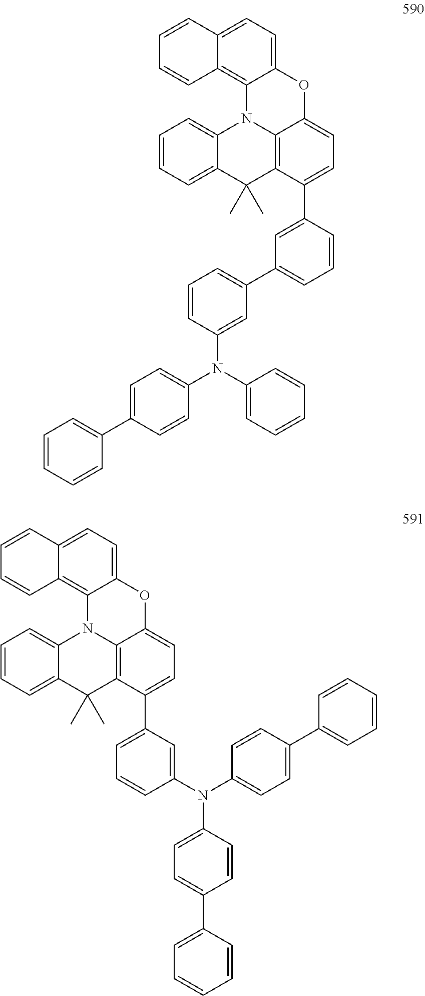

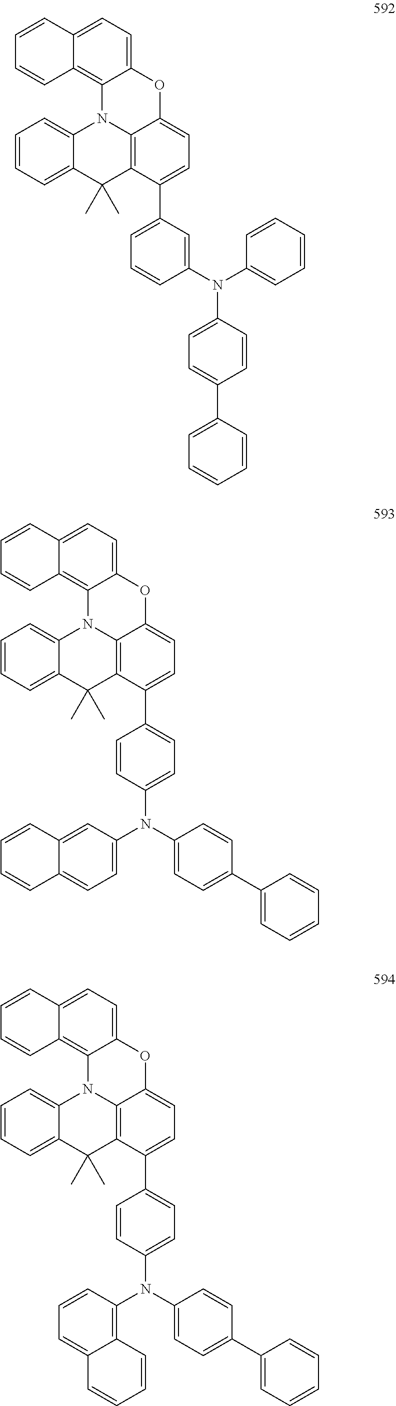 [SMILES: CC1(C)c2ccccc2N2c3c(ccc(-c4cccc(N(c5ccccc5)c5ccc(-c6ccccc6)cc5)c4)c31)Oc1ccc3ccccc3c12 ; CC1(C)c2ccccc2N2c3c(ccc(-c4ccc(N(c5ccc(-c6ccccc6)cc5)c5ccc6ccccc6c5)cc4)c31)Oc1ccc3ccccc3c12 ; CC1(C)c2ccccc2N2c3c(ccc(-c4ccc(N(c5ccc(-c6ccccc6)cc5)c5cccc6ccccc56)cc4)c31)Oc1ccc3ccccc3c12]

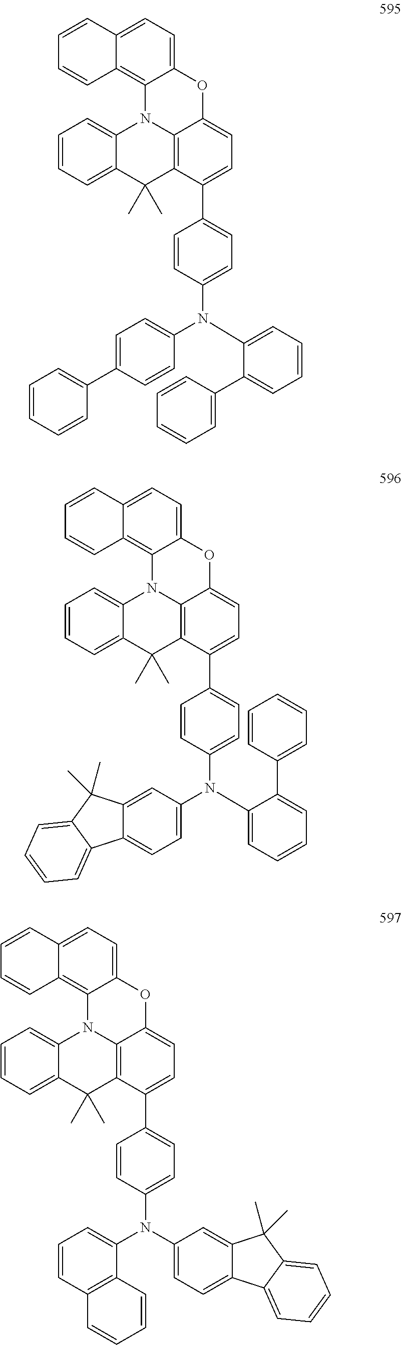 [SMILES: CC1(C)c2ccccc2N2c3c(ccc(-c4ccc(N(c5ccc(-c6ccccc6)cc5)c5ccccc5-c5ccccc5)cc4)c31)Oc1ccc3ccccc3c12 ; CC1(C)c2ccccc2-c2ccc(N(c3ccc(-c4ccc5c6c4C(C)(C)c4ccccc4N6c4c(ccc6ccccc46)O5)cc3)c3ccccc3-c3ccccc3)cc21 ; CC1(C)c2ccccc2-c2ccc(N(c3ccc(-c4ccc5c6c4C(C)(C)c4ccccc4N6c4c(ccc6ccccc46)O5)cc3)c3cccc4ccccc34)cc21]

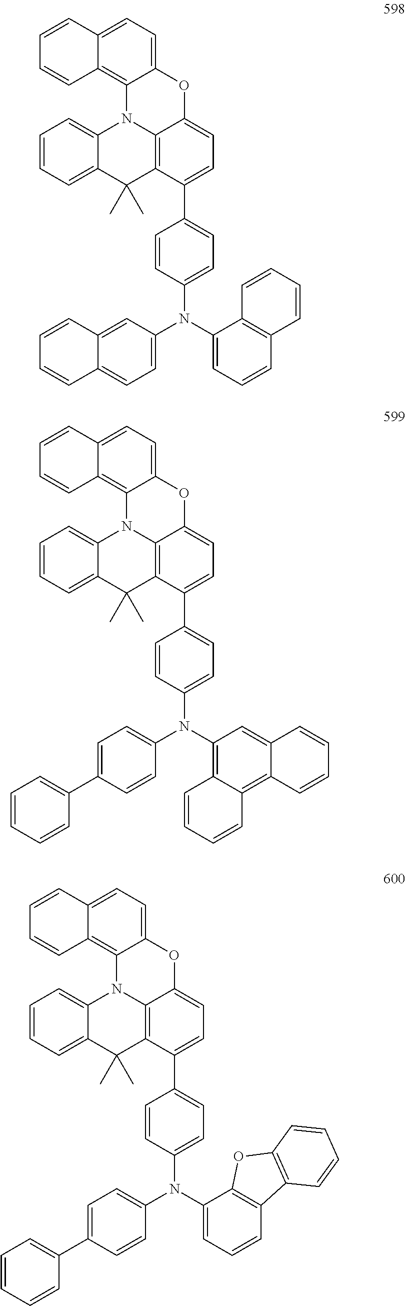

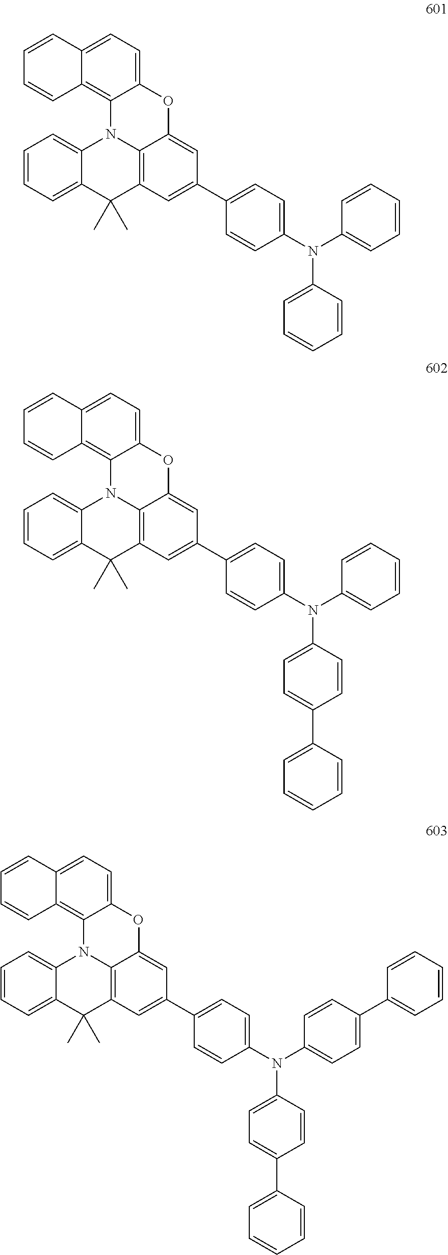 [SMILES: CC1(C)c2ccccc2N2c3c(cc(-c4ccc(N(c5ccccc5)c5ccccc5)cc4)cc31)Oc1ccc3ccccc3c12 ; CC1(C)c2ccccc2N2c3c(cc(-c4ccc(N(c5ccccc5)c5ccc(-c6ccccc6)cc5)cc4)cc31)Oc1ccc3ccccc3c12 ; CC1(C)c2ccccc2N2c3c(cc(-c4ccc(N(c5ccc(-c6ccccc6)cc5)c5ccc(-c6ccccc6)cc5)cc4)cc31)Oc1ccc3ccccc3c12]

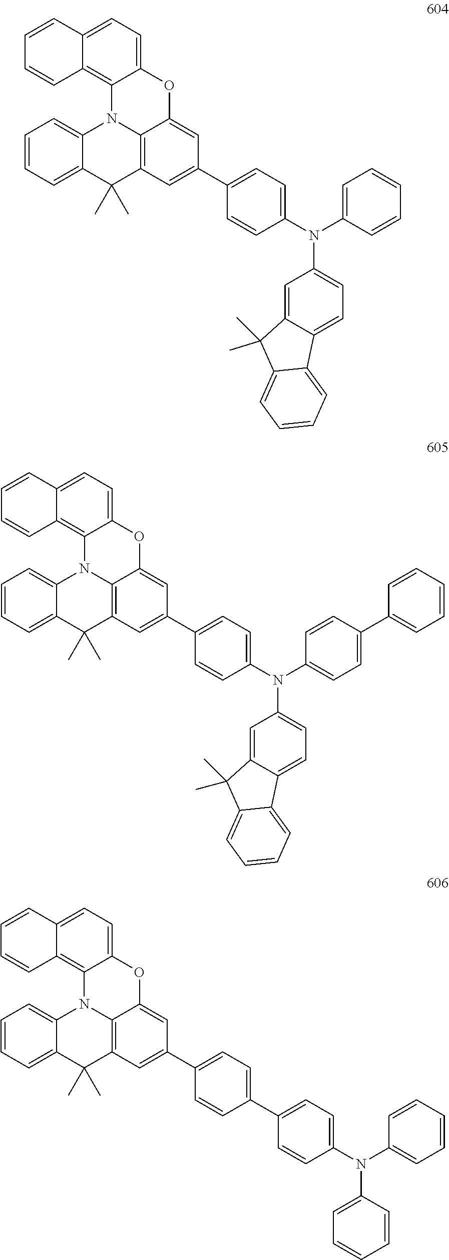 [SMILES: CC1(C)c2ccccc2-c2ccc(N(c3ccccc3)c3ccc(-c4cc5c6c(c4)C(C)(C)c4ccccc4N6c4c(ccc6ccccc46)O5)cc3)cc21 ; CC1(C)c2ccccc2-c2ccc(N(c3ccc(-c4ccccc4)cc3)c3ccc(-c4cc5c6c(c4)C(C)(C)c4ccccc4N6c4c(ccc6ccccc46)O5)cc3)cc21 ; CC1(C)c2ccccc2N2c3c(cc(-c4ccc(-c5ccc(N(c6ccccc6)c6ccccc6)cc5)cc4)cc31)Oc1ccc3ccccc3c12]

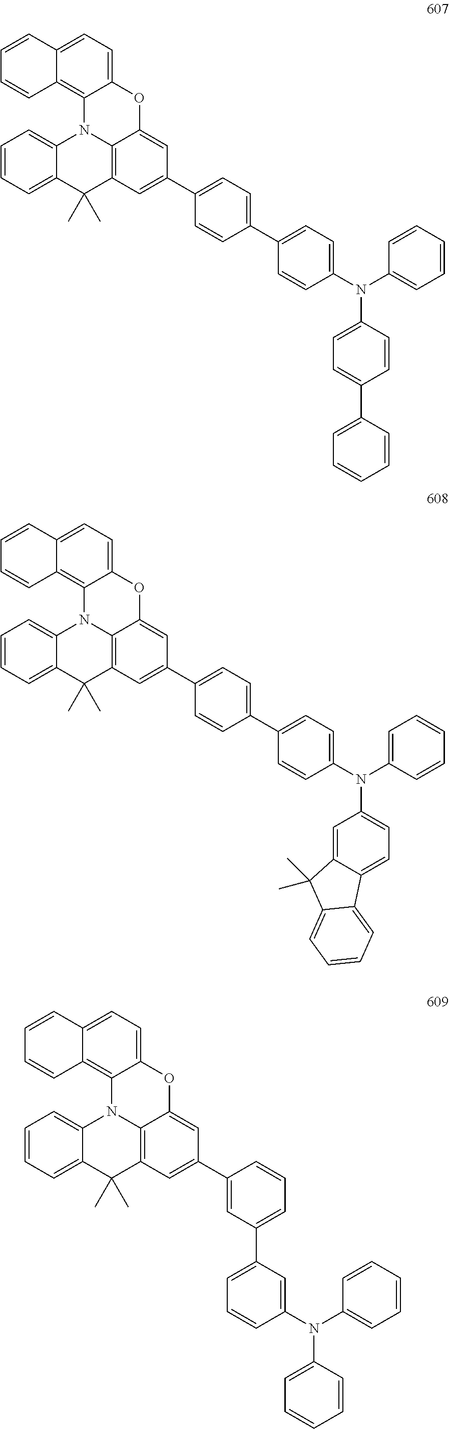 [SMILES: CC1(C)c2ccccc2N2c3c(cc(-c4ccc(-c5ccc(N(c6ccccc6)c6ccc(-c7ccccc7)cc6)cc5)cc4)cc31)Oc1ccc3ccccc3c12 ; CC1(C)c2ccccc2-c2ccc(N(c3ccccc3)c3ccc(-c4ccc(-c5cc6c7c(c5)C(C)(C)c5ccccc5N7c5c(ccc7ccccc57)O6)cc4)cc3)cc21 ; CC1(C)c2ccccc2N2c3c(cc(-c4cccc(-c5cccc(N(c6ccccc6)c6ccccc6)c5)c4)cc31)Oc1ccc3ccccc3c12]

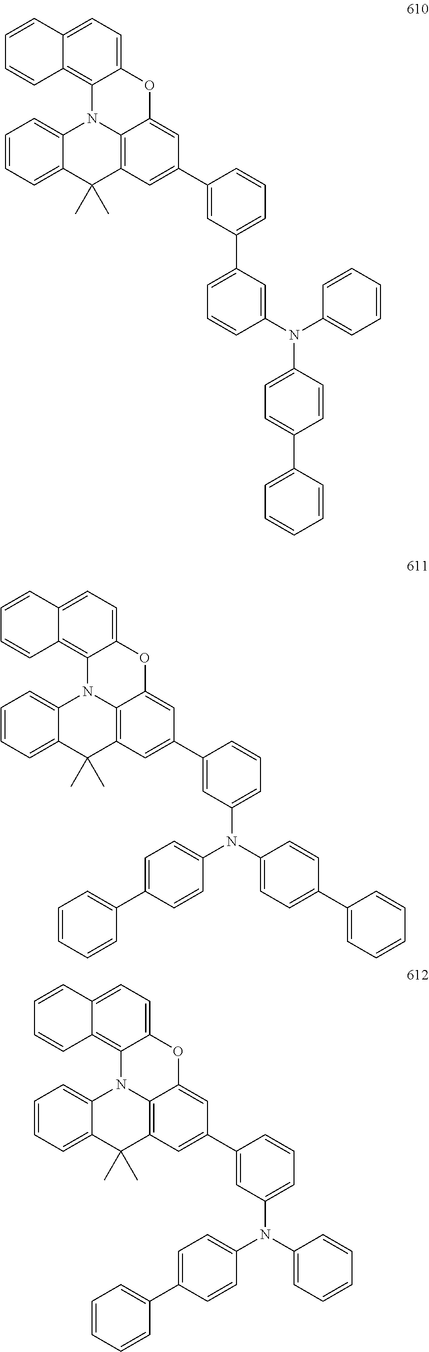

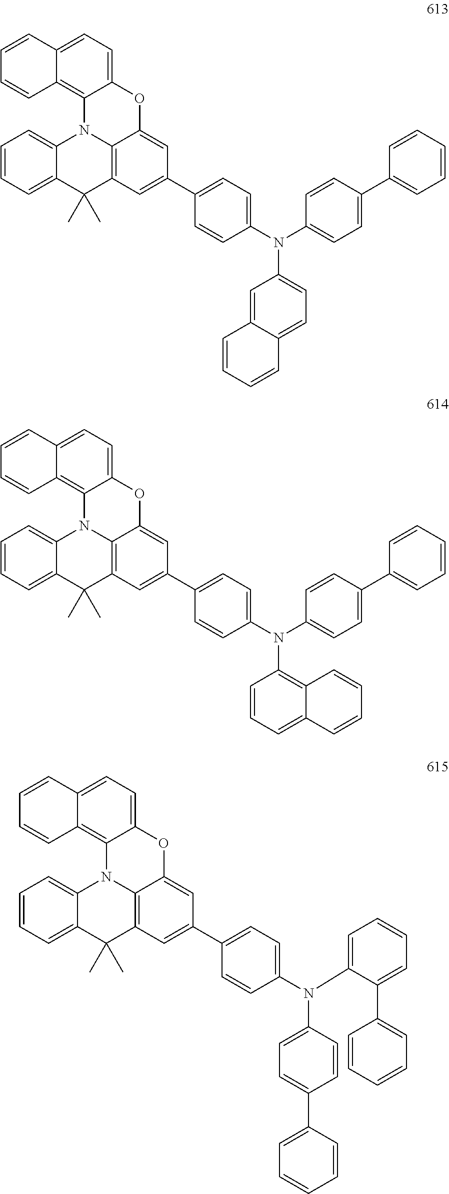 [SMILES: CC1(C)c2ccccc2N2c3c(cc(-c4ccc(N(c5ccc(-c6ccccc6)cc5)c5ccc6ccccc6c5)cc4)cc31)Oc1ccc3ccccc3c12 ; CC1(C)c2ccccc2N2c3c(cc(-c4ccc(N(c5ccc(-c6ccccc6)cc5)c5cccc6ccccc56)cc4)cc31)Oc1ccc3ccccc3c12 ; CC1(C)c2ccccc2N2c3c(cc(-c4ccc(N(c5ccc(-c6ccccc6)cc5)c5ccccc5-c5ccccc5)cc4)cc31)Oc1ccc3ccccc3c12]

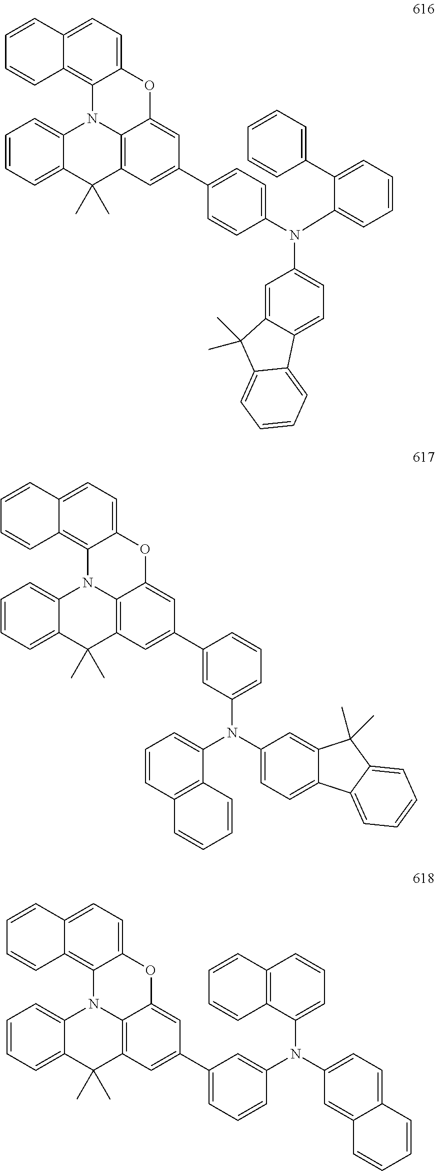 [SMILES: CC1(C)c2ccccc2-c2ccc(N(c3ccc(-c4cc5c6c(c4)C(C)(C)c4ccccc4N6c4c(ccc6ccccc46)O5)cc3)c3ccccc3-c3ccccc3)cc21 ; CC1(C)c2ccccc2-c2ccc(N(c3cccc(-c4cc5c6c(c4)C(C)(C)c4ccccc4N6c4c(ccc6ccccc46)O5)c3)c3cccc4ccccc34)cc21 ; CC1(C)c2ccccc2N2c3c(cc(-c4cccc(N(c5ccc6ccccc6c5)c5cccc6ccccc56)c4)cc31)Oc1ccc3ccccc3c12]

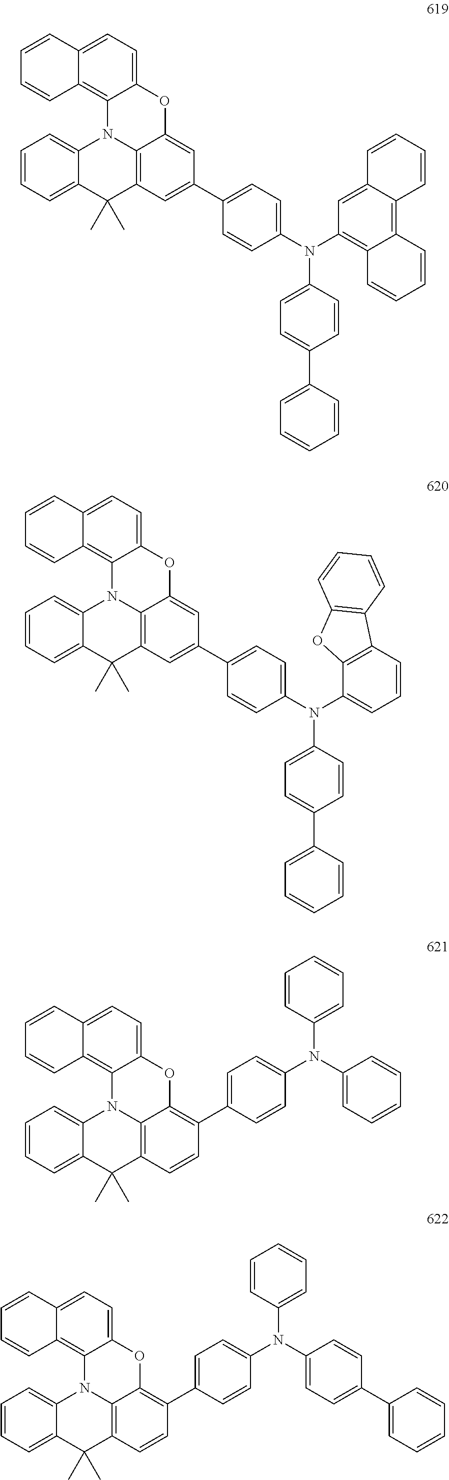

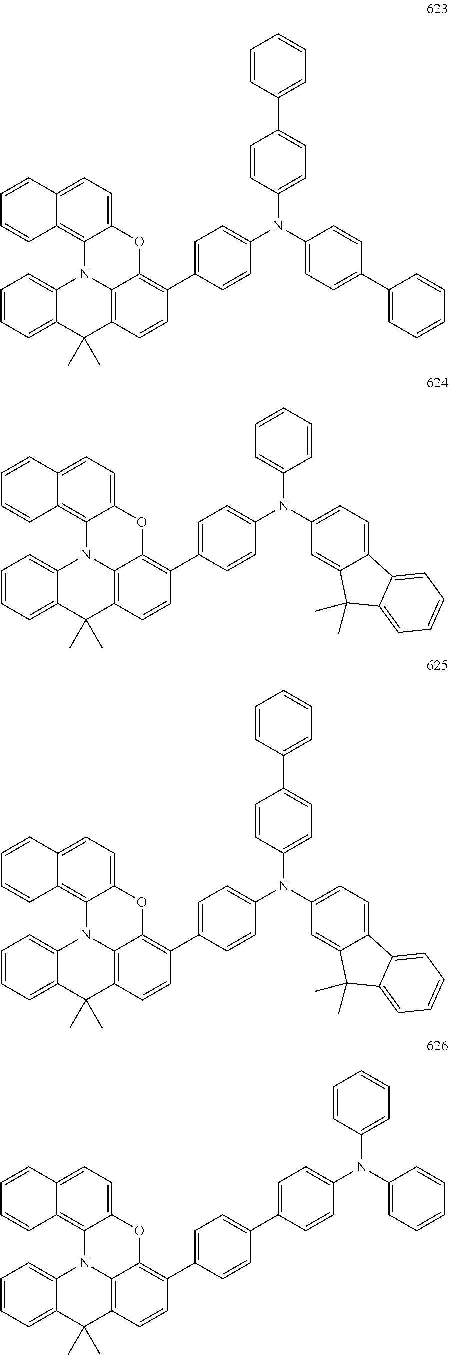 [SMILES: CC1(C)c2ccccc2N2c3c1ccc(-c1ccc(N(c4ccc(-c5ccccc5)cc4)c4ccc(-c5ccccc5)cc4)cc1)c3Oc1ccc3ccccc3c12 ; CC1(C)c2ccccc2-c2ccc(N(c3ccccc3)c3ccc(-c4ccc5c6c4Oc4ccc7ccccc7c4N6c4ccccc4C5(C)C)cc3)cc21 ; CC1(C)c2ccccc2-c2ccc(N(c3ccc(-c4ccccc4)cc3)c3ccc(-c4ccc5c6c4Oc4ccc7ccccc7c4N6c4ccccc4C5(C)C)cc3)cc21 ; CC1(C)c2ccccc2N2c3c1ccc(-c1ccc(-c4ccc(N(c5ccccc5)c5ccccc5)cc4)cc1)c3Oc1ccc3ccccc3c12]

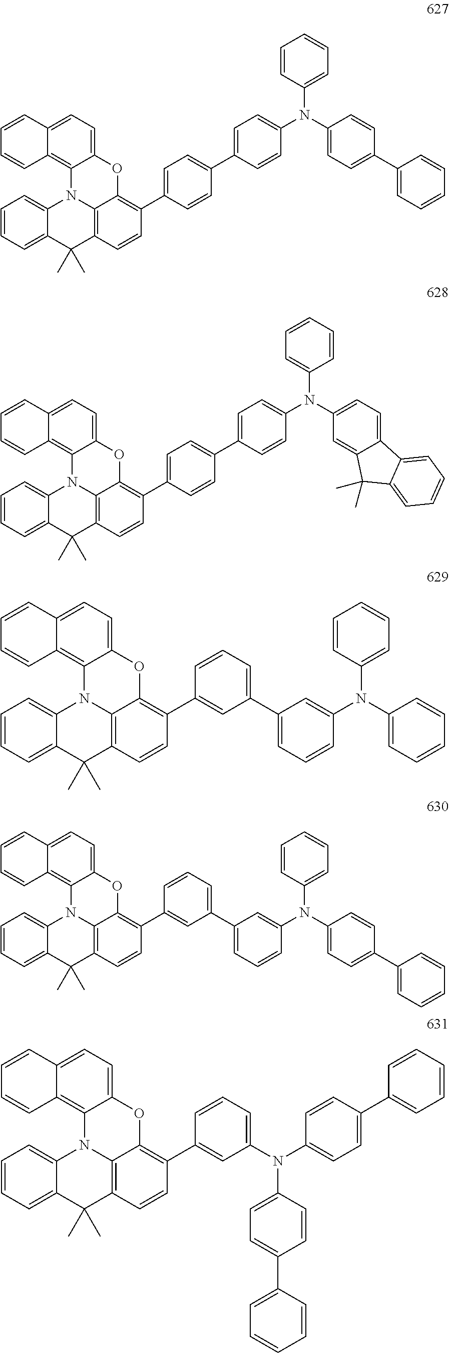 [SMILES: CC1(C)c2ccccc2N2c3c1ccc(-c1ccc(-c4ccc(N(c5ccccc5)c5ccc(-c6ccccc6)cc5)cc4)cc1)c3Oc1ccc3ccccc3c12 ; CC1(C)c2ccccc2-c2ccc(N(c3ccccc3)c3ccc(-c4ccc(-c5ccc6c7c5Oc5ccc8ccccc8c5N7c5ccccc5C6(C)C)cc4)cc3)cc21 ; CC1(C)c2ccccc2N2c3c1ccc(-c1cccc(-c4cccc(N(c5ccccc5)c5ccccc5)c4)c1)c3Oc1ccc3ccccc3c12 ; CC1(C)c2ccccc2N2c3c1ccc(-c1cccc(-c4cccc(N(c5ccccc5)c5ccc(-c6ccccc6)cc5)c4)c1)c3Oc1ccc3ccccc3c12 ; CC1(C)c2ccccc2N2c3c1ccc(-c1cccc(N(c4ccc(-c5ccccc5)cc4)c4ccc(-c5ccccc5)cc4)c1)c3Oc1ccc3ccccc3c12]

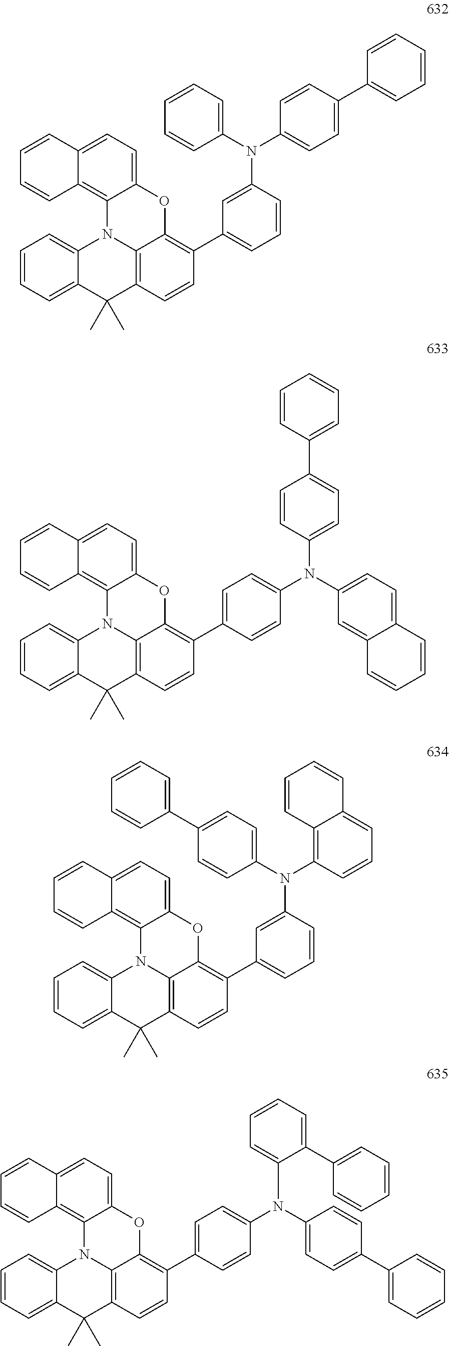

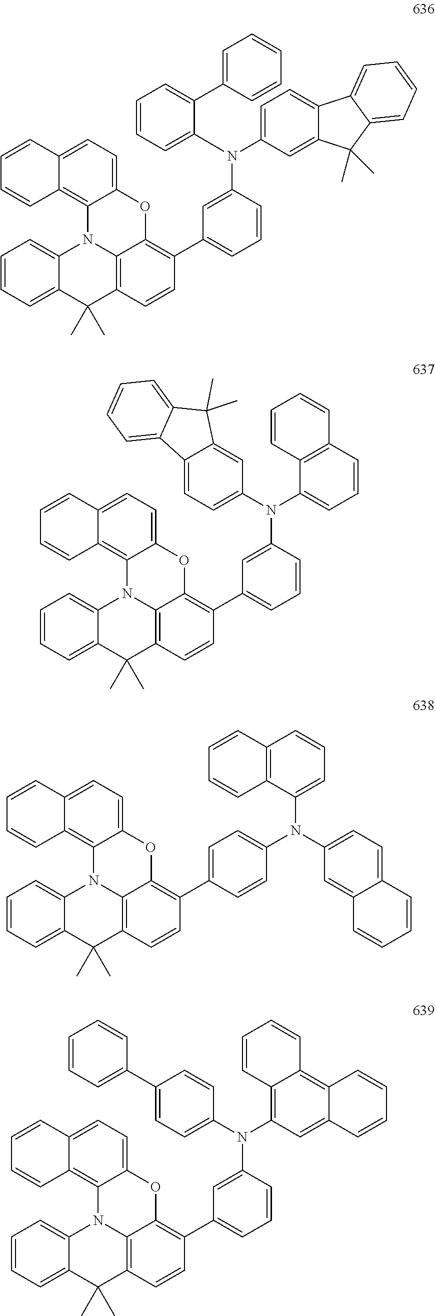

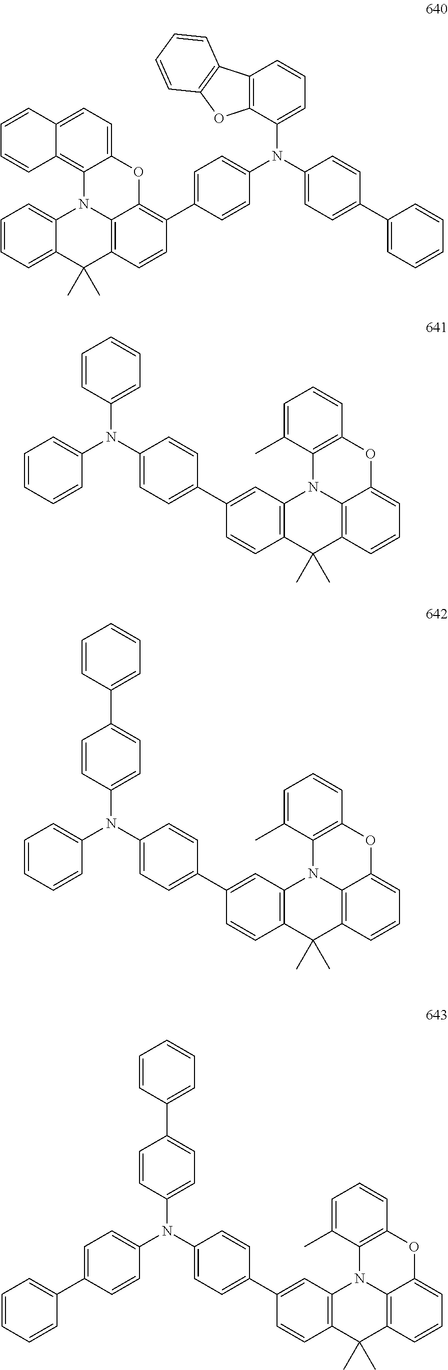

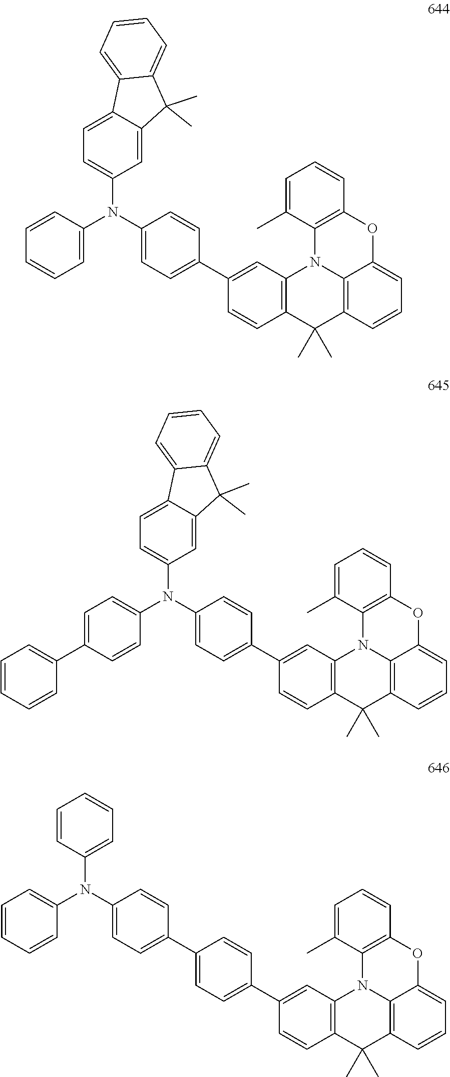 [SMILES: Cc1cccc2c1N1c3cc(-c4ccc(N(c5ccccc5)c5ccc6c(c5)C(C)(C)c5ccccc5-6)cc4)ccc3C(C)(C)c3cccc(c31)O2 ; Cc1cccc2c1N1c3cc(-c4ccc(N(c5ccc(-c6ccccc6)cc5)c5ccc6c(c5)C(C)(C)c5ccccc5-6)cc4)ccc3C(C)(C)c3cccc(c31)O2 ; Cc1cccc2c1N1c3cc(-c4ccc(-c5ccc(N(c6ccccc6)c6ccccc6)cc5)cc4)ccc3C(C)(C)c3cccc(c31)O2]

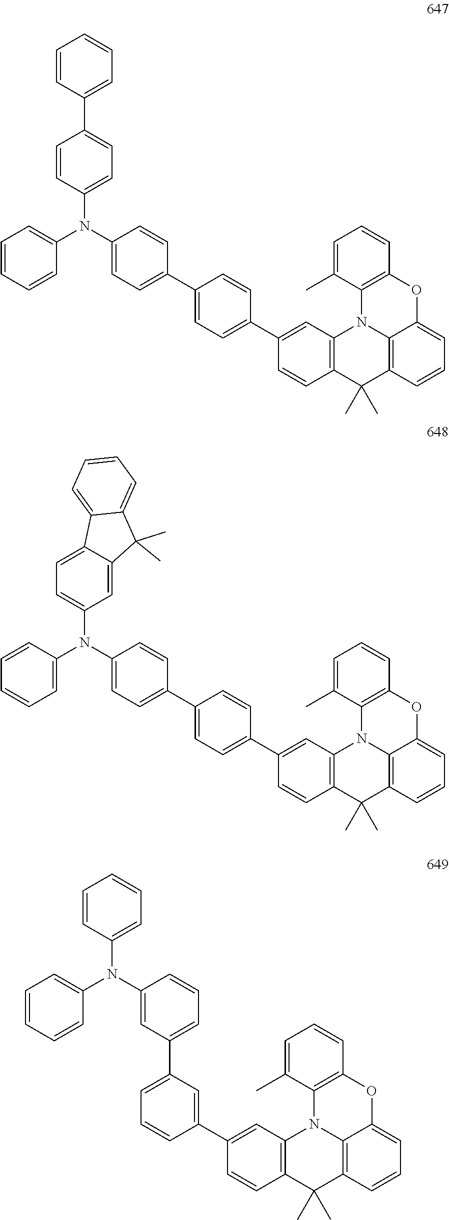 [SMILES: Cc1cccc2c1N1c3cc(-c4ccc(-c5ccc(N(c6ccccc6)c6ccc(-c7ccccc7)cc6)cc5)cc4)ccc3C(C)(C)c3cccc(c31)O2 ; Cc1cccc2c1N1c3cc(-c4ccc(-c5ccc(N(c6ccccc6)c6ccc7c(c6)C(C)(C)c6ccccc6-7)cc5)cc4)ccc3C(C)(C)c3cccc(c31)O2 ; Cc1cccc2c1N1c3cc(-c4cccc(-c5cccc(N(c6ccccc6)c6ccccc6)c5)c4)ccc3C(C)(C)c3cccc(c31)O2]

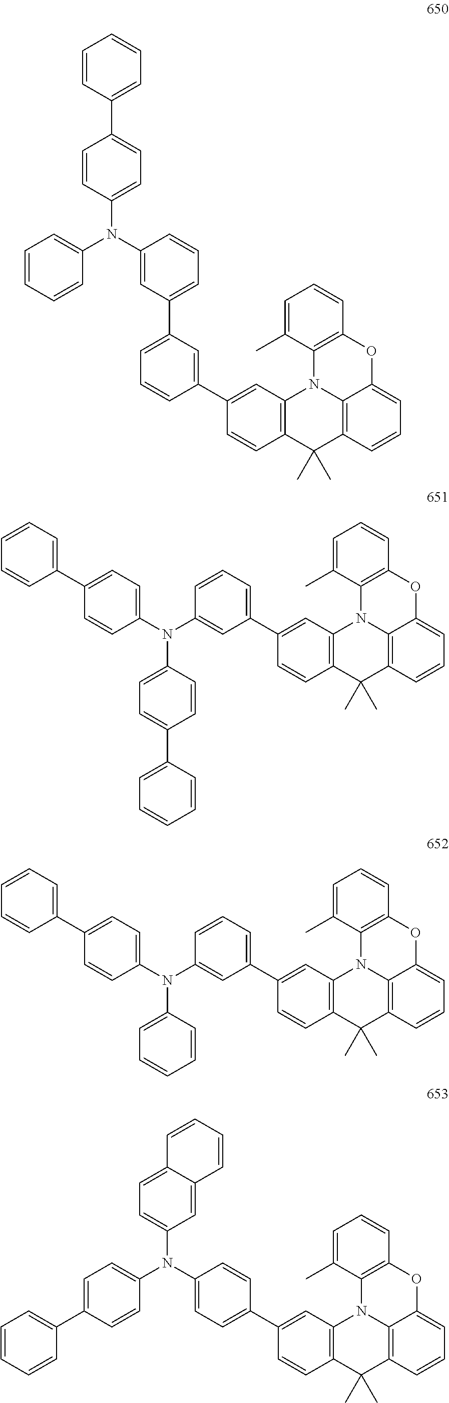 [SMILES: Cc1cccc2c1N1c3cc(-c4cccc(-c5cccc(N(c6ccccc6)c6ccc(-c7ccccc7)cc6)c5)c4)ccc3C(C)(C)c3cccc(c31)O2 ; Cc1cccc2c1N1c3cc(-c4cccc(N(c5ccc(-c6ccccc6)cc5)c5ccc(-c6ccccc6)cc5)c4)ccc3C(C)(C)c3cccc(c31)O2 ; Cc1cccc2c1N1c3cc(-c4cccc(N(c5ccccc5)c5ccc(-c6ccccc6)cc5)c4)ccc3C(C)(C)c3cccc(c31)O2 ; Cc1cccc2c1N1c3cc(-c4ccc(N(c5ccc(-c6ccccc6)cc5)c5ccc6ccccc6c5)cc4)ccc3C(C)(C)c3cccc(c31)O2]

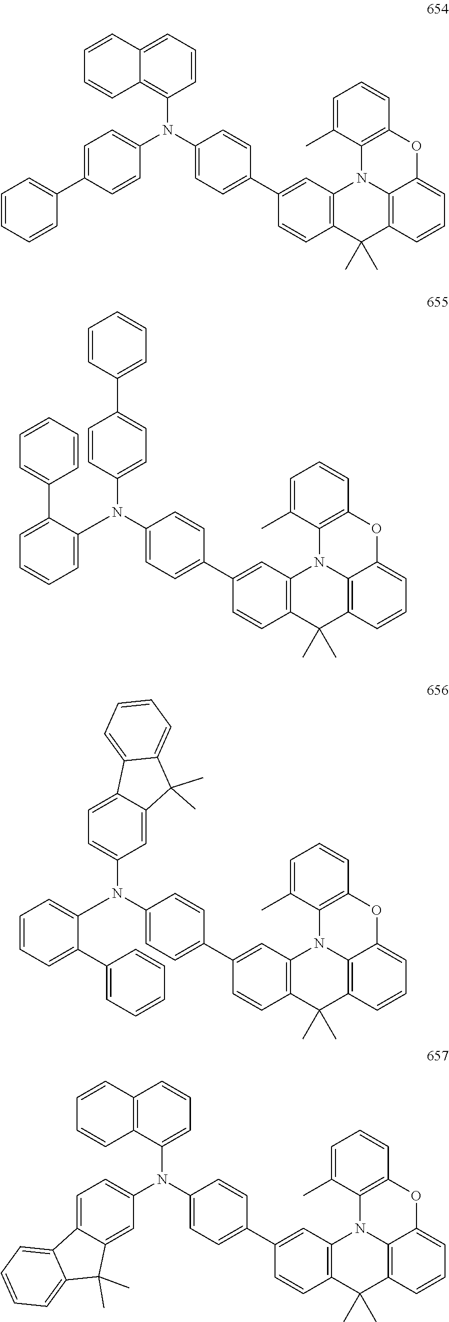 [SMILES: Cc1cccc2c1N1c3cc(-c4ccc(N(c5ccc(-c6ccccc6)cc5)c5cccc6ccccc56)cc4)ccc3C(C)(C)c3cccc(c31)O2 ; Cc1cccc2c1N1c3cc(-c4ccc(N(c5ccc(-c6ccccc6)cc5)c5ccccc5-c5ccccc5)cc4)ccc3C(C)(C)c3cccc(c31)O2 ; Cc1cccc2c1N1c3cc(-c4ccc(N(c5ccc6c(c5)C(C)(C)c5ccccc5-6)c5ccccc5-c5ccccc5)cc4)ccc3C(C)(C)c3cccc(c31)O2 ; Cc1cccc2c1N1c3cc(-c4ccc(N(c5ccc6c(c5)C(C)(C)c5ccccc5-6)c5cccc6ccccc56)cc4)ccc3C(C)(C)c3cccc(c31)O2]

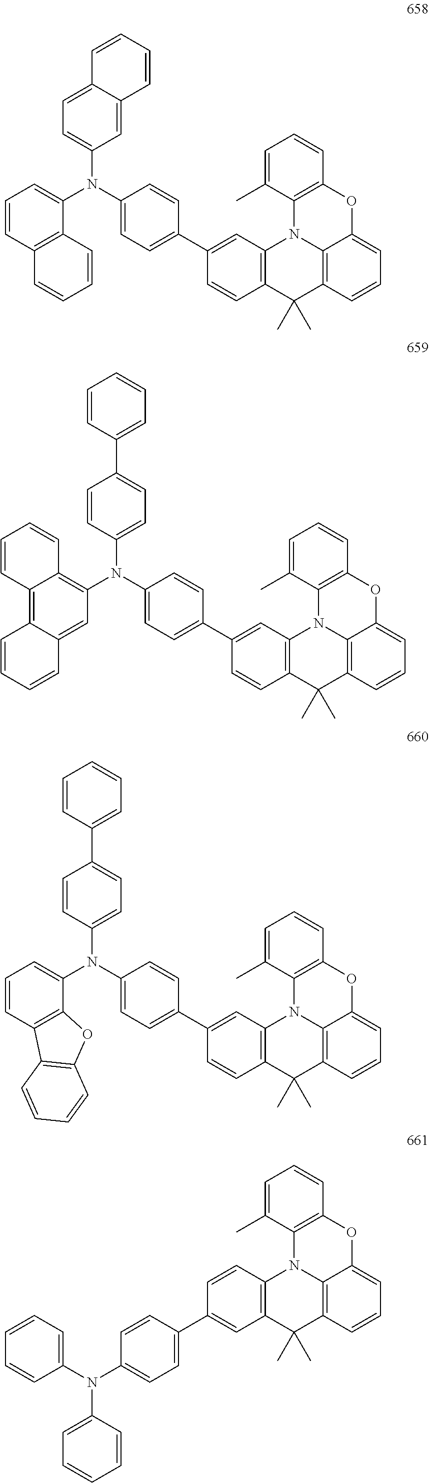 [SMILES: Cc1cccc2c1N1c3cc(-c4ccc(N(c5ccc6ccccc6c5)c5cccc6ccccc56)cc4)ccc3C(C)(C)c3cccc(c31)O2 ; Cc1cccc2c1N1c3cc(-c4ccc(N(c5ccc(-c6ccccc6)cc5)c5cc6ccccc6c6ccccc56)cc4)ccc3C(C)(C)c3cccc(c31)O2 ; Cc1cccc2c1N1c3cc(-c4ccc(N(c5ccc(-c6ccccc6)cc5)c5cccc6c5oc5ccccc56)cc4)ccc3C(C)(C)c3cccc(c31)O2 ; Cc1cccc2c1N1c3ccc(-c4ccc(N(c5ccccc5)c5ccccc5)cc4)cc3C(C)(C)c3cccc(c31)O2]

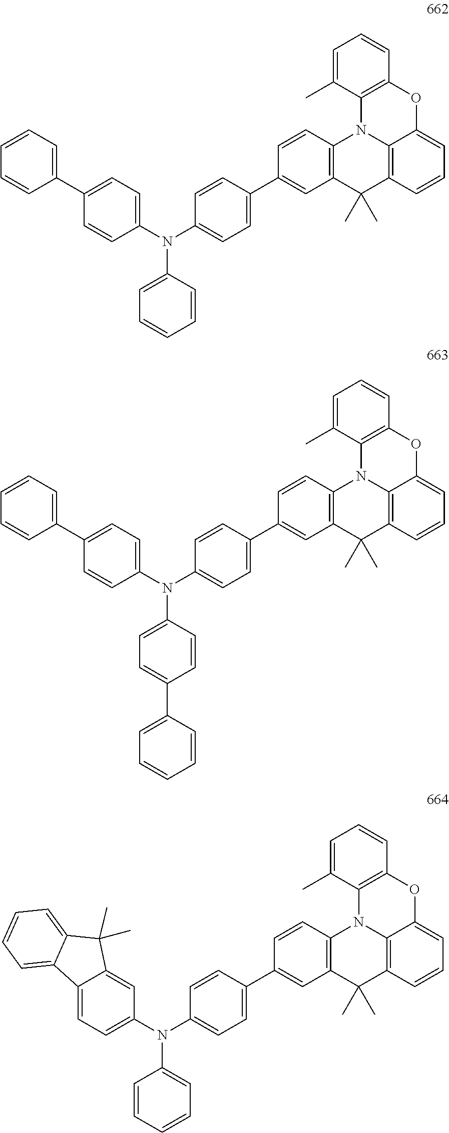 [SMILES: Cc1cccc2c1N1c3ccc(-c4ccc(N(c5ccccc5)c5ccc(-c6ccccc6)cc5)cc4)cc3C(C)(C)c3cccc(c31)O2 ; Cc1cccc2c1N1c3ccc(-c4ccc(N(c5ccc(-c6ccccc6)cc5)c5ccc(-c6ccccc6)cc5)cc4)cc3C(C)(C)c3cccc(c31)O2 ; Cc1cccc2c1N1c3ccc(-c4ccc(N(c5ccccc5)c5ccc6c(c5)C(C)(C)c5ccccc5-6)cc4)cc3C(C)(C)c3cccc(c31)O2]

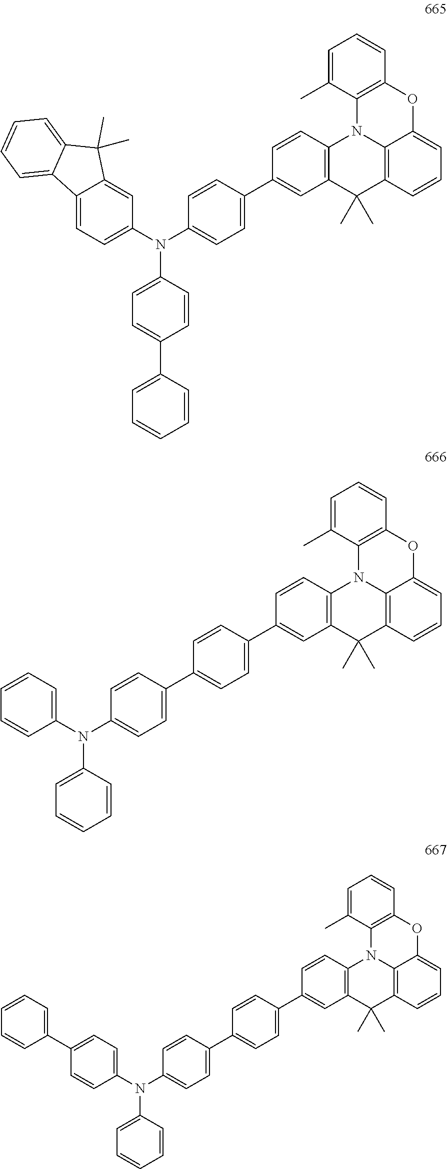 [SMILES: Cc1cccc2c1N1c3ccc(-c4ccc(N(c5ccc(-c6ccccc6)cc5)c5ccc6c(c5)C(C)(C)c5ccccc5-6)cc4)cc3C(C)(C)c3cccc(c31)O2 ; Cc1cccc2c1N1c3ccc(-c4ccc(-c5ccc(N(c6ccccc6)c6ccccc6)cc5)cc4)cc3C(C)(C)c3cccc(c31)O2 ; Cc1cccc2c1N1c3ccc(-c4ccc(-c5ccc(N(c6ccccc6)c6ccc(-c7ccccc7)cc6)cc5)cc4)cc3C(C)(C)c3cccc(c31)O2]

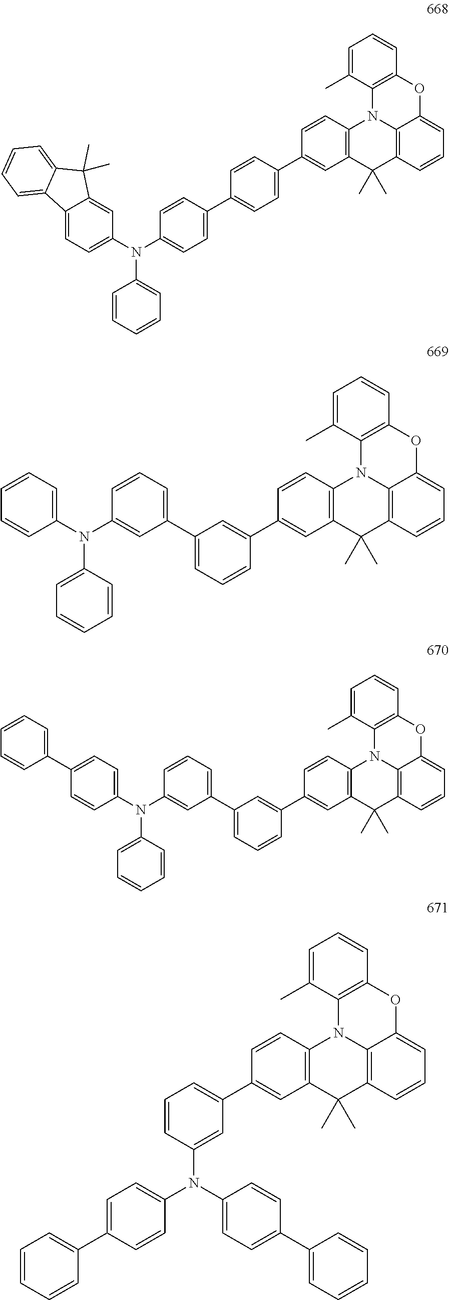 [SMILES: Cc1cccc2c1N1c3ccc(-c4ccc(-c5ccc(N(c6ccccc6)c6ccc7c(c6)C(C)(C)c6ccccc6-7)cc5)cc4)cc3C(C)(C)c3cccc(c31)O2 ; Cc1cccc2c1N1c3ccc(-c4cccc(-c5cccc(N(c6ccccc6)c6ccccc6)c5)c4)cc3C(C)(C)c3cccc(c31)O2 ; Cc1cccc2c1N1c3ccc(-c4cccc(-c5cccc(N(c6ccccc6)c6ccc(-c7ccccc7)cc6)c5)c4)cc3C(C)(C)c3cccc(c31)O2 ; Cc1cccc2c1N1c3ccc(-c4cccc(N(c5ccc(-c6ccccc6)cc5)c5ccc(-c6ccccc6)cc5)c4)cc3C(C)(C)c3cccc(c31)O2]

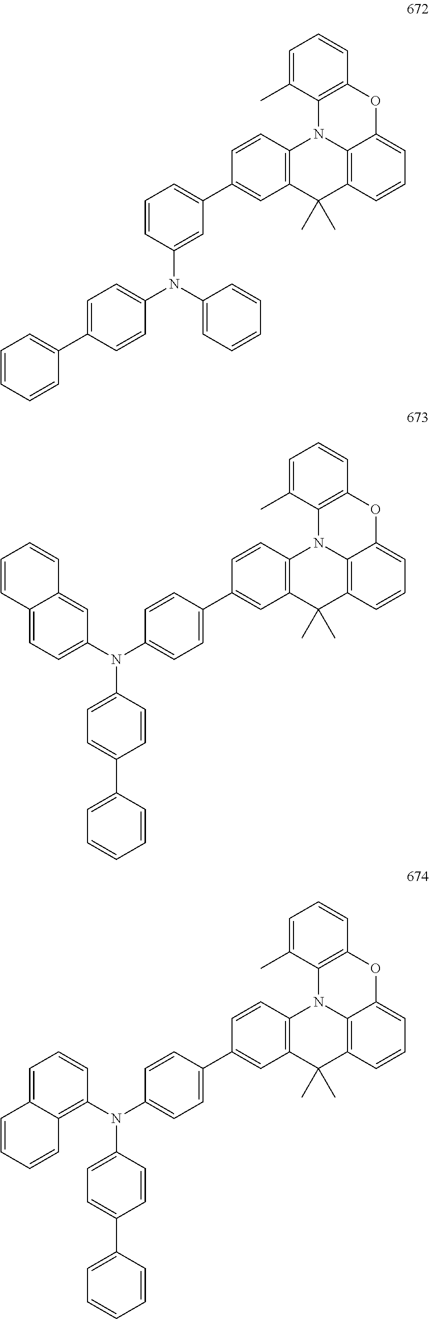 [SMILES: Cc1cccc2c1N1c3ccc(-c4cccc(N(c5ccccc5)c5ccc(-c6ccccc6)cc5)c4)cc3C(C)(C)c3cccc(c31)O2 ; Cc1cccc2c1N1c3ccc(-c4ccc(N(c5ccc(-c6ccccc6)cc5)c5ccc6ccccc6c5)cc4)cc3C(C)(C)c3cccc(c31)O2 ; Cc1cccc2c1N1c3ccc(-c4ccc(N(c5ccc(-c6ccccc6)cc5)c5cccc6ccccc56)cc4)cc3C(C)(C)c3cccc(c31)O2]

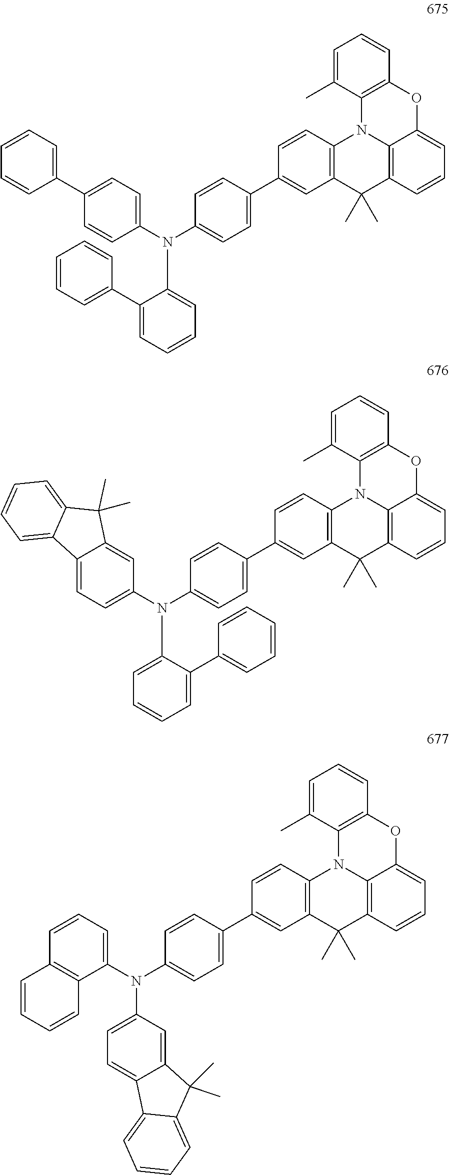 [SMILES: Cc1cccc2c1N1c3ccc(-c4ccc(N(c5ccc(-c6ccccc6)cc5)c5ccccc5-c5ccccc5)cc4)cc3C(C)(C)c3cccc(c31)O2 ; Cc1cccc2c1N1c3ccc(-c4ccc(N(c5ccc6c(c5)C(C)(C)c5ccccc5-6)c5ccccc5-c5ccccc5)cc4)cc3C(C)(C)c3cccc(c31)O2 ; Cc1cccc2c1N1c3ccc(-c4ccc(N(c5ccc6c(c5)C(C)(C)c5ccccc5-6)c5cccc6ccccc56)cc4)cc3C(C)(C)c3cccc(c31)O2]

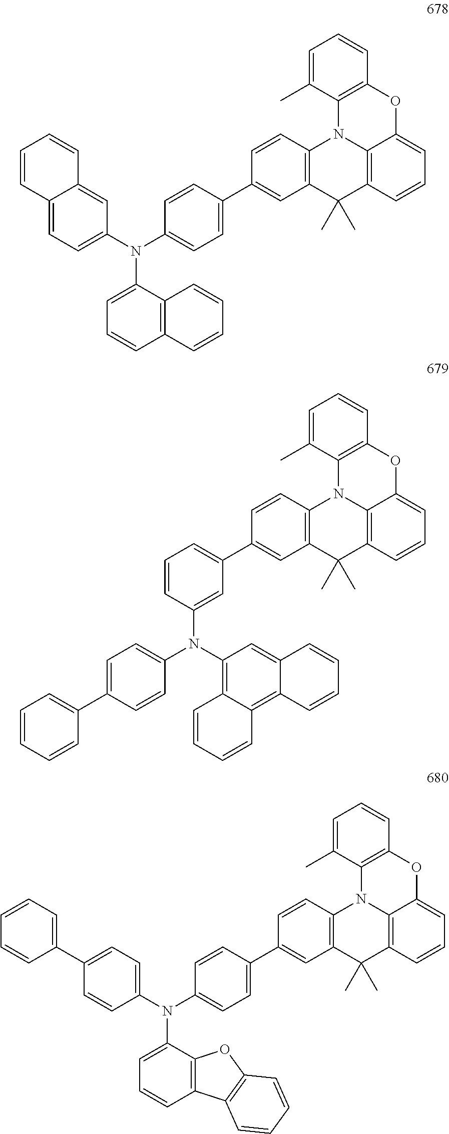 [SMILES: Cc1cccc2c1N1c3ccc(-c4ccc(N(c5ccc6ccccc6c5)c5cccc6ccccc56)cc4)cc3C(C)(C)c3cccc(c31)O2 ; Cc1cccc2c1N1c3ccc(-c4cccc(N(c5ccc(-c6ccccc6)cc5)c5cc6ccccc6c6ccccc56)c4)cc3C(C)(C)c3cccc(c31)O2 ; Cc1cccc2c1N1c3ccc(-c4ccc(N(c5ccc(-c6ccccc6)cc5)c5cccc6c5oc5ccccc56)cc4)cc3C(C)(C)c3cccc(c31)O2]

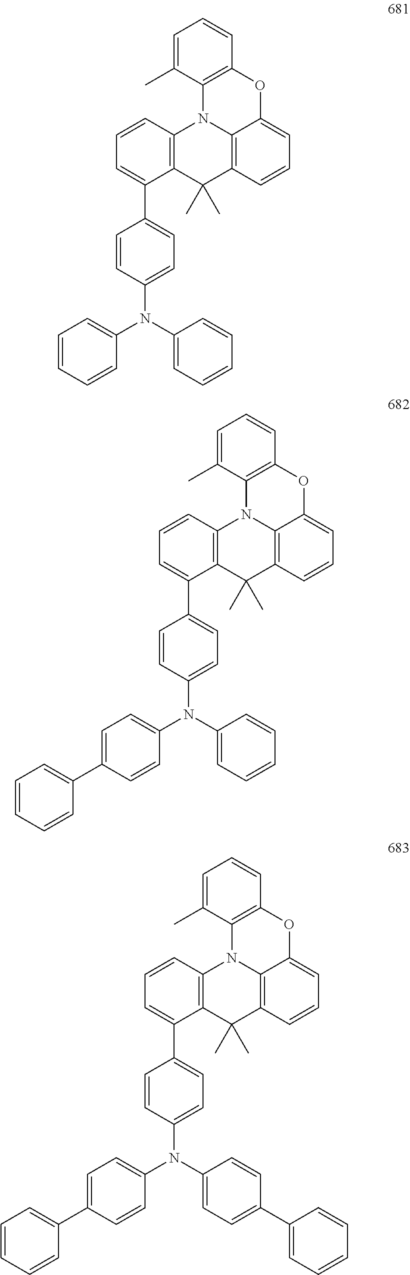 [SMILES: Cc1cccc2c1N1c3cccc(-c4ccc(N(c5ccccc5)c5ccccc5)cc4)c3C(C)(C)c3cccc(c31)O2 ; Cc1cccc2c1N1c3cccc(-c4ccc(N(c5ccccc5)c5ccc(-c6ccccc6)cc5)cc4)c3C(C)(C)c3cccc(c31)O2 ; Cc1cccc2c1N1c3cccc(-c4ccc(N(c5ccc(-c6ccccc6)cc5)c5ccc(-c6ccccc6)cc5)cc4)c3C(C)(C)c3cccc(c31)O2]

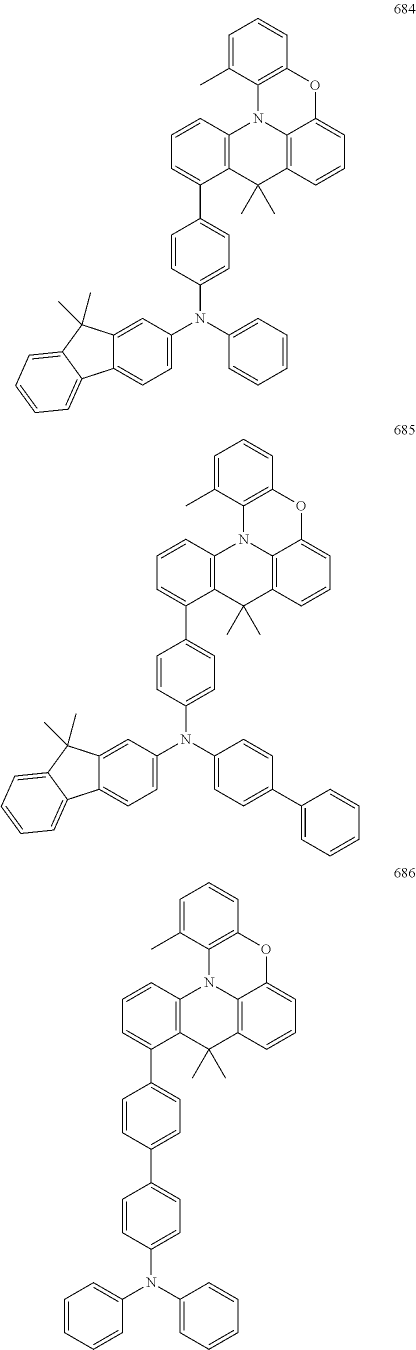 [SMILES: Cc1cccc2c1N1c3cccc(-c4ccc(N(c5ccccc5)c5ccc6c(c5)C(C)(C)c5ccccc5-6)cc4)c3C(C)(C)c3cccc(c31)O2 ; Cc1cccc2c1N1c3cccc(-c4ccc(N(c5ccc(-c6ccccc6)cc5)c5ccc6c(c5)C(C)(C)c5ccccc5-6)cc4)c3C(C)(C)c3cccc(c31)O2 ; Cc1cccc2c1N1c3cccc(-c4ccc(-c5ccc(N(c6ccccc6)c6ccccc6)cc5)cc4)c3C(C)(C)c3cccc(c31)O2]

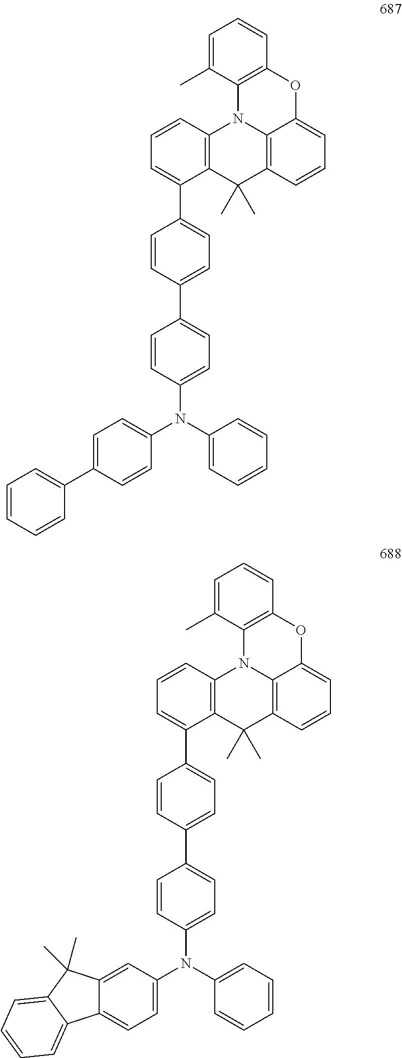 [SMILES: Cc1cccc2c1N1c3cccc(-c4ccc(-c5ccc(N(c6ccccc6)c6ccc(-c7ccccc7)cc6)cc5)cc4)c3C(C)(C)c3cccc(c31)O2 ; Cc1cccc2c1N1c3cccc(-c4ccc(-c5ccc(N(c6ccccc6)c6ccc7c(c6)C(C)(C)c6ccccc6-7)cc5)cc4)c3C(C)(C)c3cccc(c31)O2]

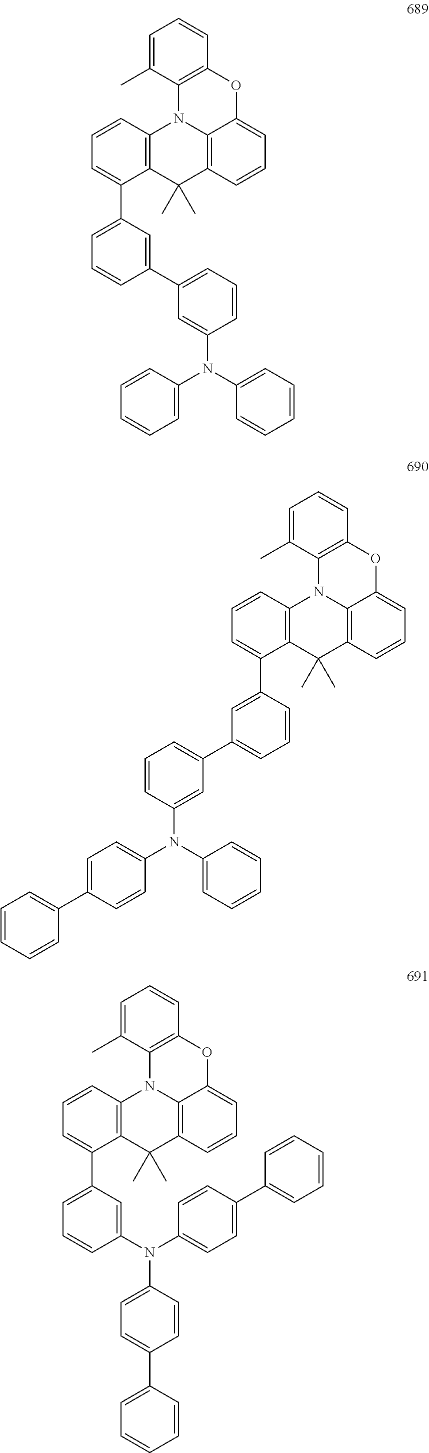 [SMILES: Cc1cccc2c1N1c3cccc(-c4cccc(-c5cccc(N(c6ccccc6)c6ccccc6)c5)c4)c3C(C)(C)c3cccc(c31)O2 ; Cc1cccc2c1N1c3cccc(-c4cccc(-c5cccc(N(c6ccccc6)c6ccc(-c7ccccc7)cc6)c5)c4)c3C(C)(C)c3cccc(c31)O2 ; Cc1cccc2c1N1c3cccc(-c4cccc(N(c5ccc(-c6ccccc6)cc5)c5ccc(-c6ccccc6)cc5)c4)c3C(C)(C)c3cccc(c31)O2]

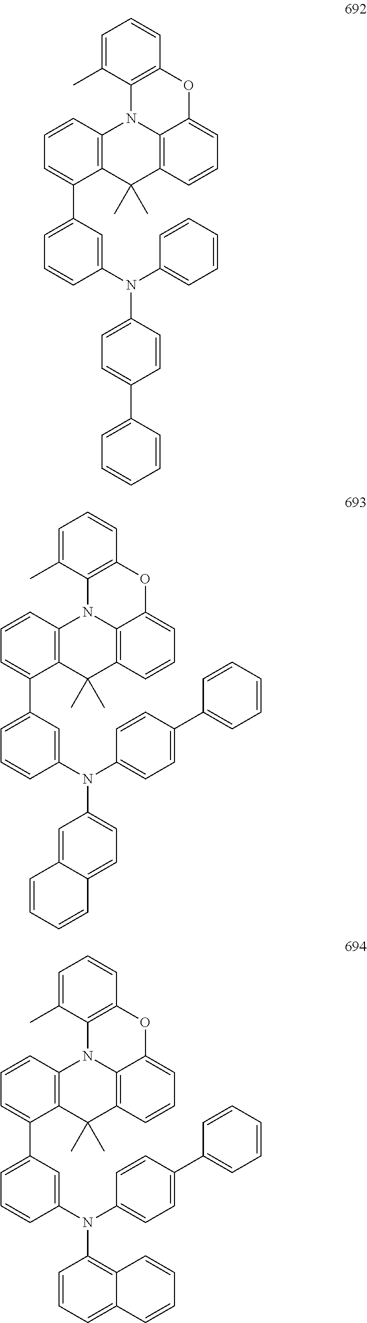

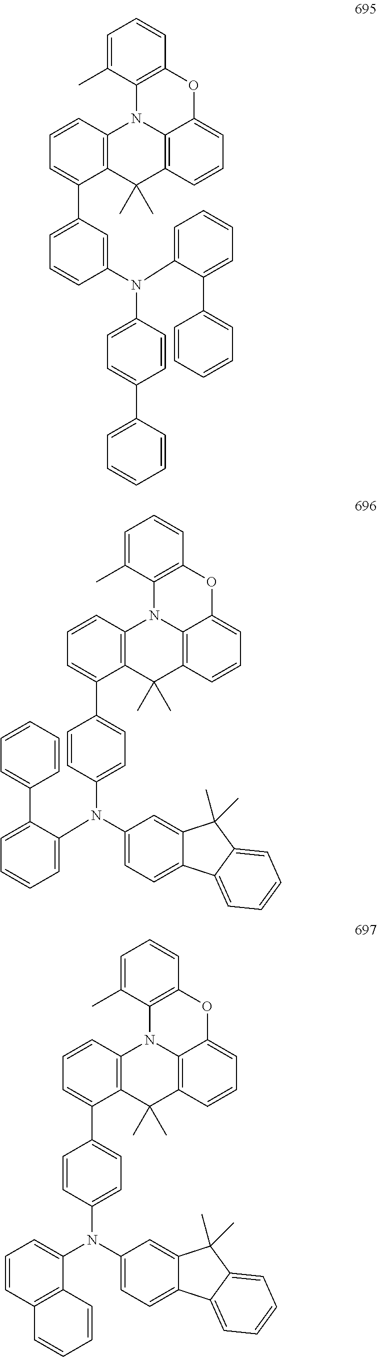

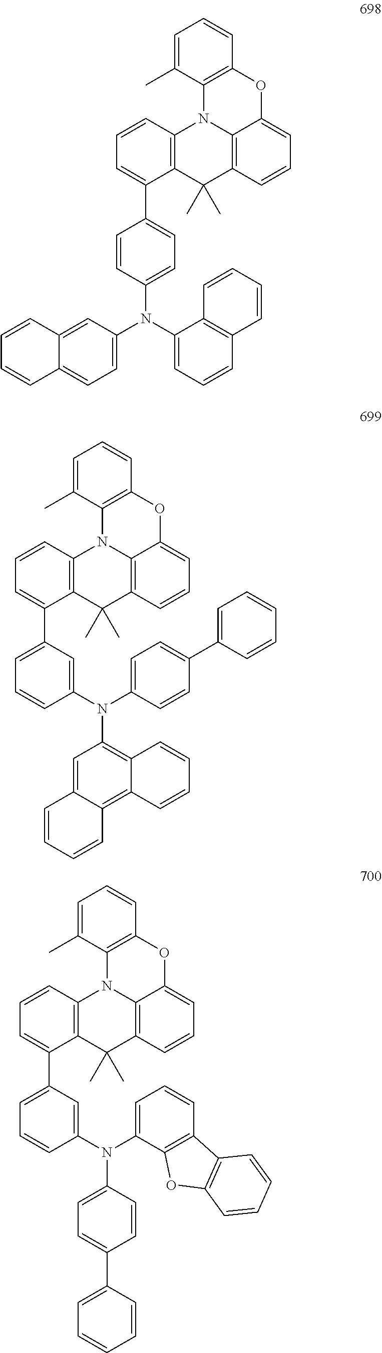

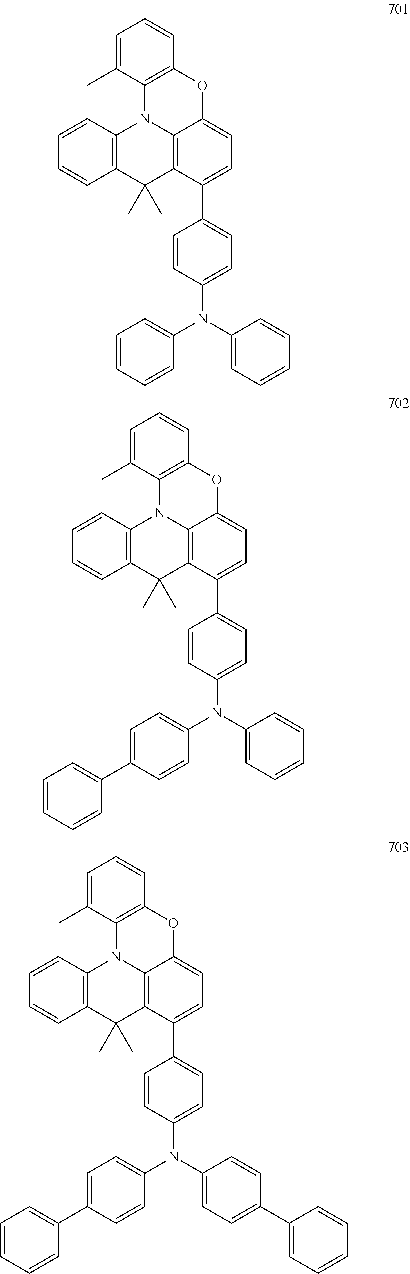

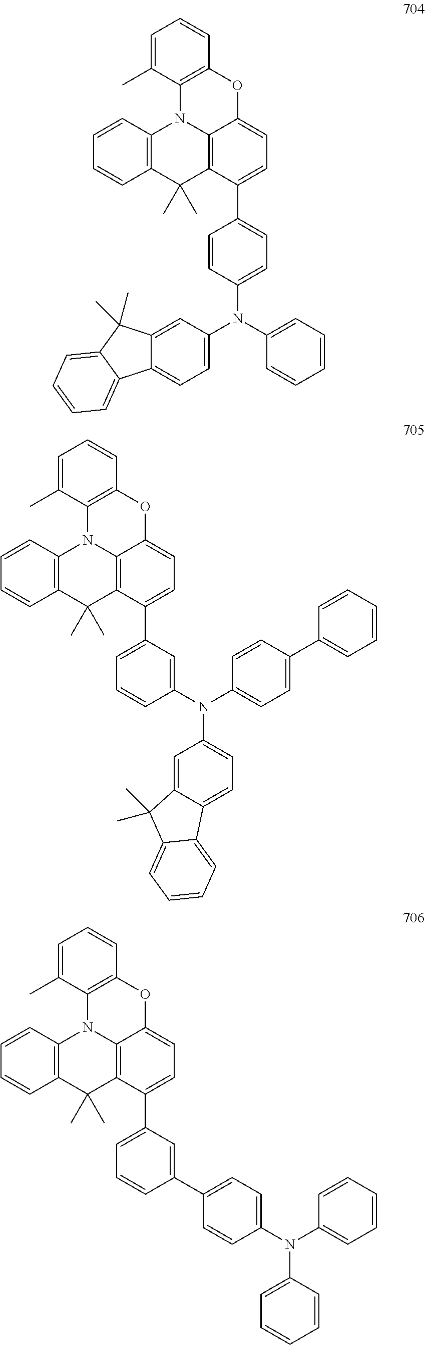

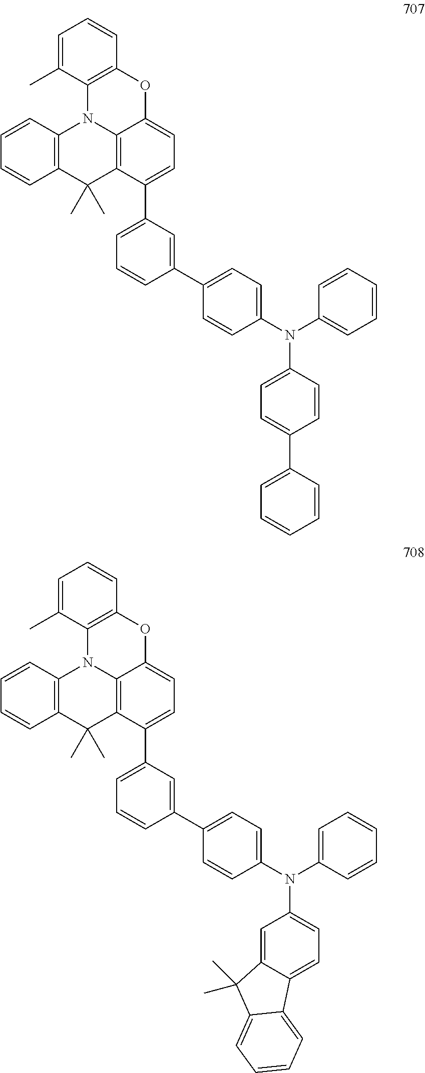 [SMILES: Cc1cccc2c1N1c3ccccc3C(C)(C)c3c(-c4cccc(-c5ccc(N(c6ccccc6)c6ccc(-c7ccccc7)cc6)cc5)c4)ccc(c31)O2 ; Cc1cccc2c1N1c3ccccc3C(C)(C)c3c(-c4cccc(-c5ccc(N(c6ccccc6)c6ccc7c(c6)C(C)(C)c6ccccc6-7)cc5)c4)ccc(c31)O2]

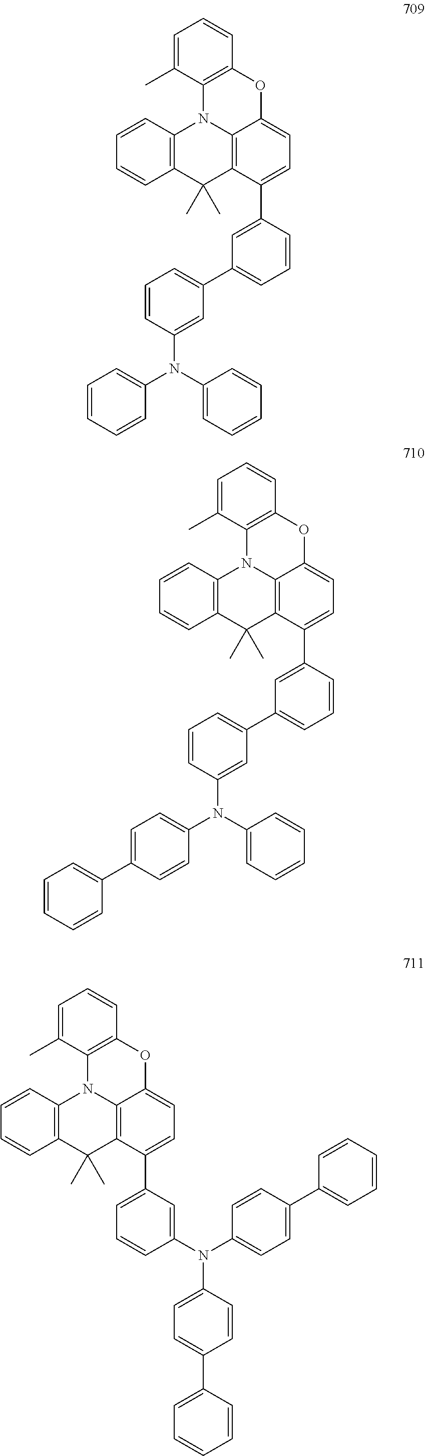

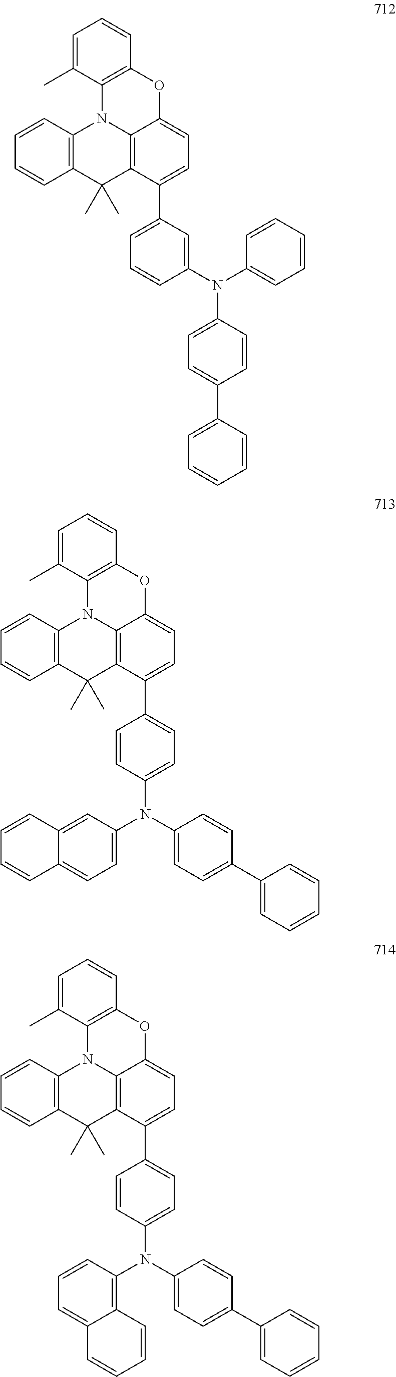 [SMILES: Cc1cccc2c1N1c3ccccc3C(C)(C)c3c(-c4cccc(N(c5ccccc5)c5ccc(-c6ccccc6)cc5)c4)ccc(c31)O2 ; Cc1cccc2c1N1c3ccccc3C(C)(C)c3c(-c4ccc(N(c5ccc(-c6ccccc6)cc5)c5ccc6ccccc6c5)cc4)ccc(c31)O2 ; Cc1cccc2c1N1c3ccccc3C(C)(C)c3c(-c4ccc(N(c5ccc(-c6ccccc6)cc5)c5cccc6ccccc56)cc4)ccc(c31)O2]

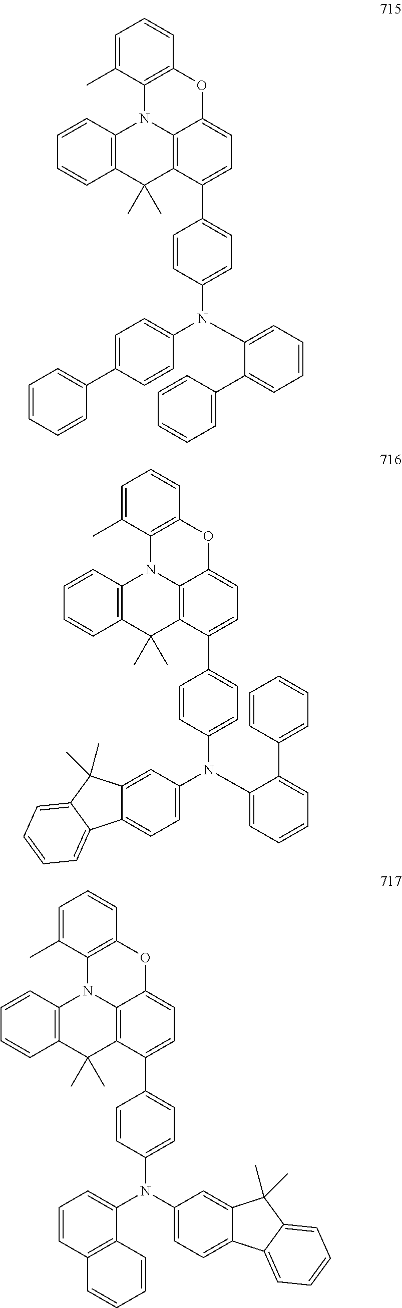

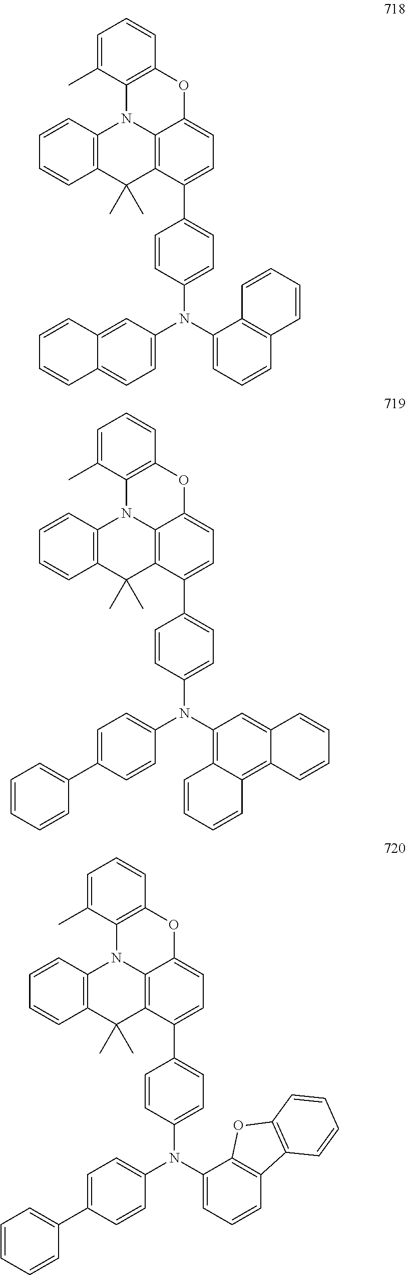

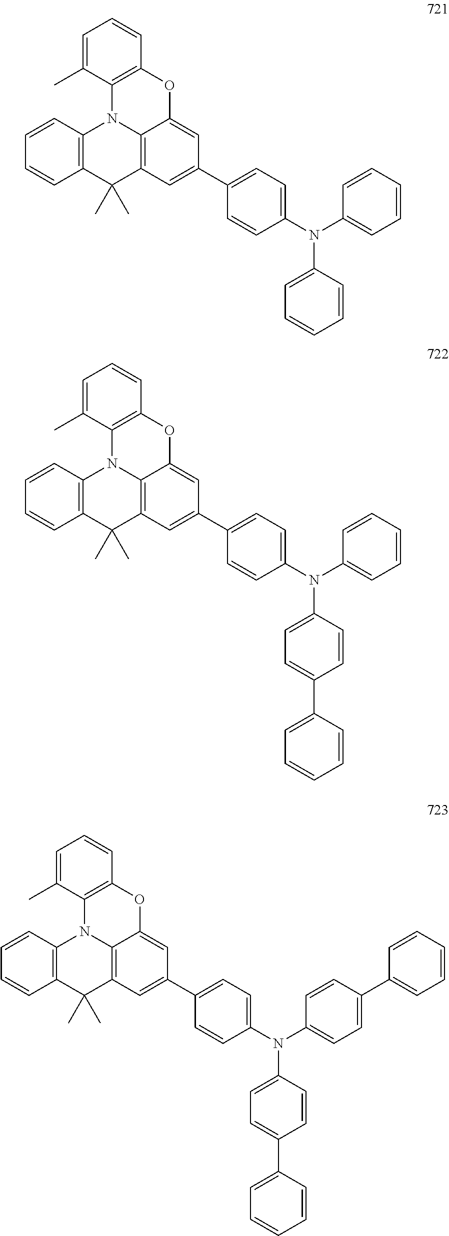 [SMILES: Cc1cccc2c1N1c3ccccc3C(C)(C)c3cc(-c4ccc(N(c5ccccc5)c5ccccc5)cc4)cc(c31)O2 ; Cc1cccc2c1N1c3ccccc3C(C)(C)c3cc(-c4ccc(N(c5ccccc5)c5ccc(-c6ccccc6)cc5)cc4)cc(c31)O2 ; Cc1cccc2c1N1c3ccccc3C(C)(C)c3cc(-c4ccc(N(c5ccc(-c6ccccc6)cc5)c5ccc(-c6ccccc6)cc5)cc4)cc(c31)O2]

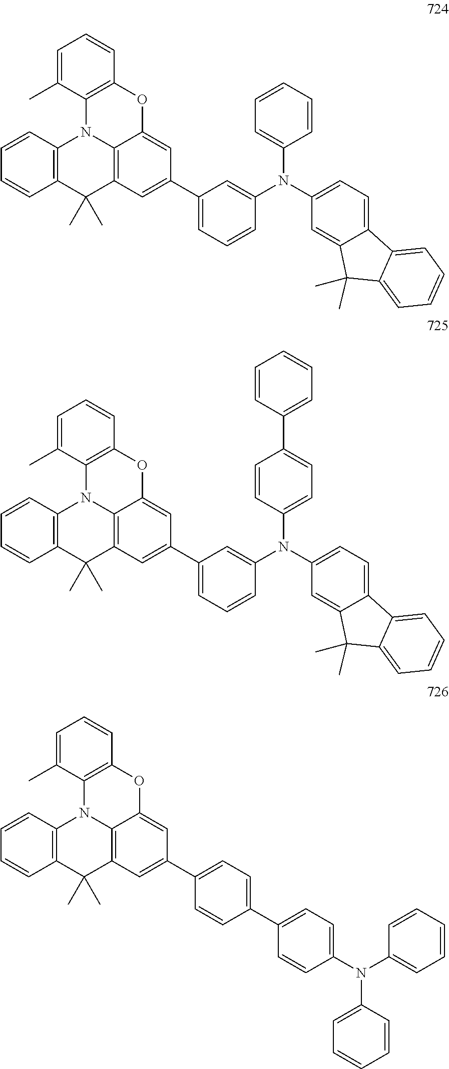 [SMILES: Cc1cccc2c1N1c3ccccc3C(C)(C)c3cc(-c4cccc(N(c5ccccc5)c5ccc6c(c5)C(C)(C)c5ccccc5-6)c4)cc(c31)O2 ; Cc1cccc2c1N1c3ccccc3C(C)(C)c3cc(-c4cccc(N(c5ccc(-c6ccccc6)cc5)c5ccc6c(c5)C(C)(C)c5ccccc5-6)c4)cc(c31)O2 ; Cc1cccc2c1N1c3ccccc3C(C)(C)c3cc(-c4ccc(-c5ccc(N(c6ccccc6)c6ccccc6)cc5)cc4)cc(c31)O2]

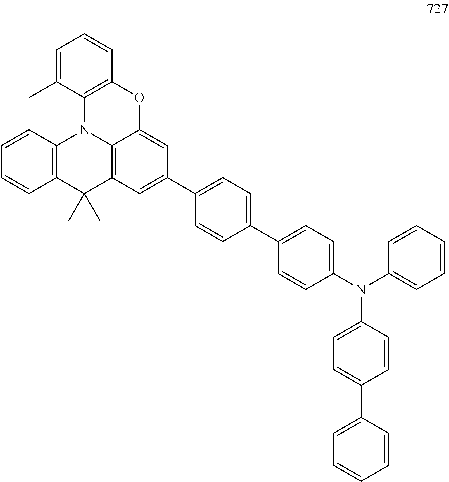 [SMILES: Cc1cccc2c1N1c3ccccc3C(C)(C)c3cc(-c4ccc(-c5ccc(N(c6ccccc6)c6ccc(-c7ccccc7)cc6)cc5)cc4)cc(c31)O2]

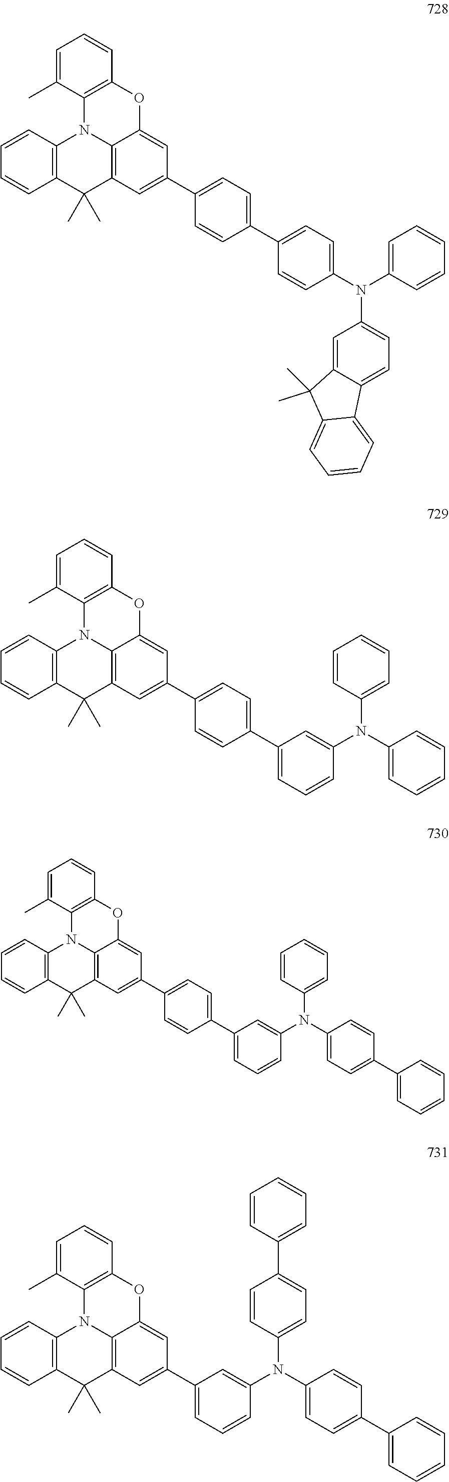 [SMILES: Cc1cccc2c1N1c3ccccc3C(C)(C)c3cc(-c4ccc(-c5ccc(N(c6ccccc6)c6ccc7c(c6)C(C)(C)c6ccccc6-7)cc5)cc4)cc(c31)O2 ; Cc1cccc2c1N1c3ccccc3C(C)(C)c3cc(-c4ccc(-c5cccc(N(c6ccccc6)c6ccccc6)c5)cc4)cc(c31)O2 ; Cc1cccc2c1N1c3ccccc3C(C)(C)c3cc(-c4ccc(-c5cccc(N(c6ccccc6)c6ccc(-c7ccccc7)cc6)c5)cc4)cc(c31)O2 ; Cc1cccc2c1N1c3ccccc3C(C)(C)c3cc(-c4cccc(N(c5ccc(-c6ccccc6)cc5)c5ccc(-c6ccccc6)cc5)c4)cc(c31)O2]

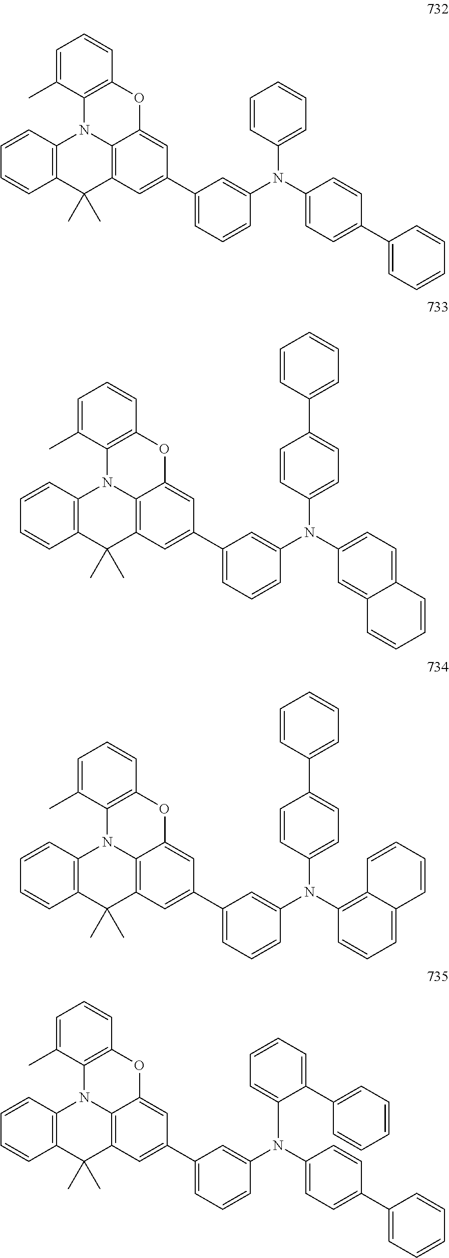 [SMILES: Cc1cccc2c1N1c3ccccc3C(C)(C)c3cc(-c4cccc(N(c5ccccc5)c5ccc(-c6ccccc6)cc5)c4)cc(c31)O2 ; Cc1cccc2c1N1c3ccccc3C(C)(C)c3cc(-c4cccc(N(c5ccc(-c6ccccc6)cc5)c5ccc6ccccc6c5)c4)cc(c31)O2 ; Cc1cccc2c1N1c3ccccc3C(C)(C)c3cc(-c4cccc(N(c5ccc(-c6ccccc6)cc5)c5cccc6ccccc56)c4)cc(c31)O2 ; Cc1cccc2c1N1c3ccccc3C(C)(C)c3cc(-c4cccc(N(c5ccc(-c6ccccc6)cc5)c5ccccc5-c5ccccc5)c4)cc(c31)O2]

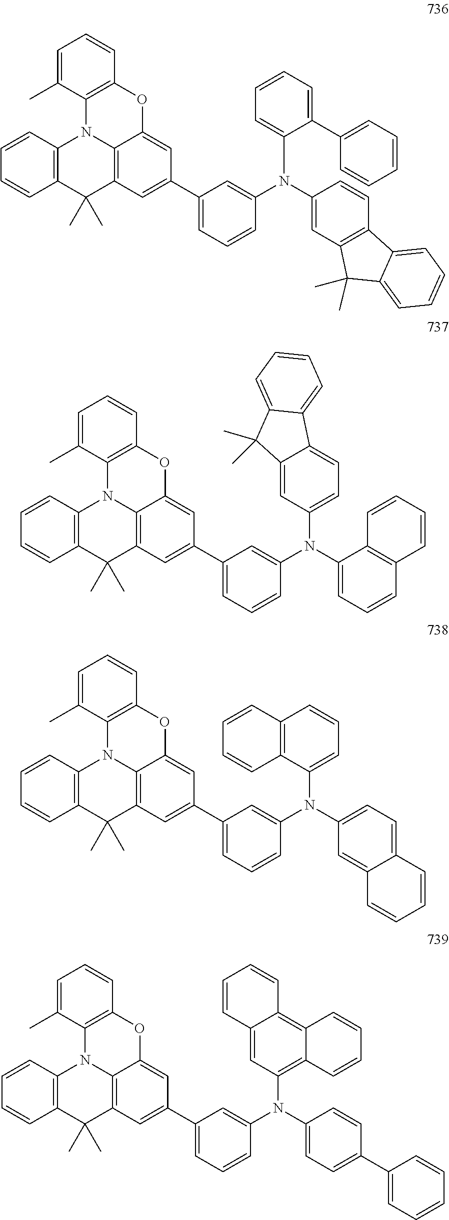 [SMILES: Cc1cccc2c1N1c3ccccc3C(C)(C)c3cc(-c4cccc(N(c5ccc6c(c5)C(C)(C)c5ccccc5-6)c5ccccc5-c5ccccc5)c4)cc(c31)O2 ; Cc1cccc2c1N1c3ccccc3C(C)(C)c3cc(-c4cccc(N(c5ccc6c(c5)C(C)(C)c5ccccc5-6)c5cccc6ccccc56)c4)cc(c31)O2 ; Cc1cccc2c1N1c3ccccc3C(C)(C)c3cc(-c4cccc(N(c5ccc6ccccc6c5)c5cccc6ccccc56)c4)cc(c31)O2 ; Cc1cccc2c1N1c3ccccc3C(C)(C)c3cc(-c4cccc(N(c5ccc(-c6ccccc6)cc5)c5cc6ccccc6c6ccccc56)c4)cc(c31)O2]

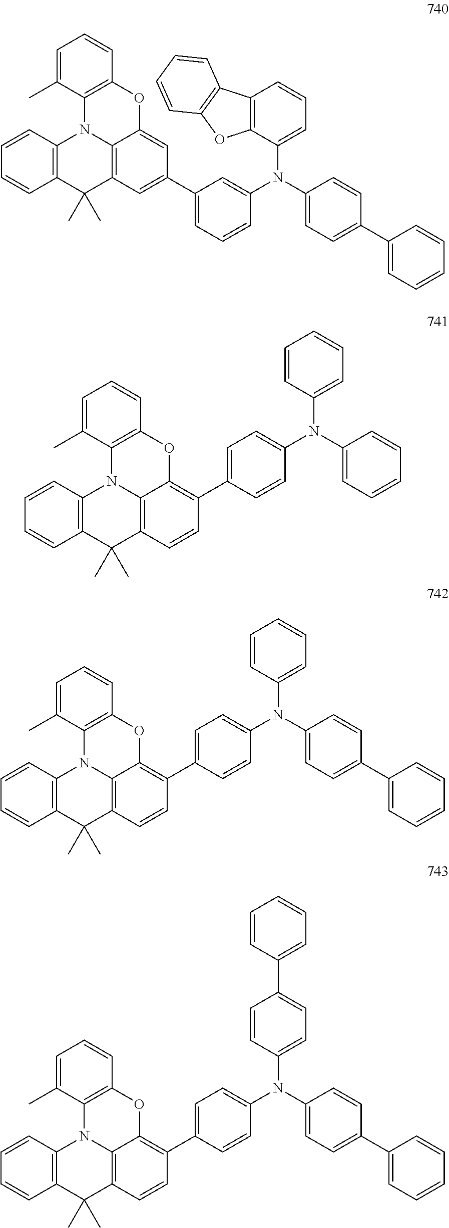 [SMILES: Cc1cccc2c1N1c3ccccc3C(C)(C)c3cc(-c4cccc(N(c5ccc(-c6ccccc6)cc5)c5cccc6c5oc5ccccc56)c4)cc(c31)O2 ; Cc1cccc2c1N1c3ccccc3C(C)(C)c3ccc(-c4ccc(N(c5ccccc5)c5ccccc5)cc4)c(c31)O2 ; Cc1cccc2c1N1c3ccccc3C(C)(C)c3ccc(-c4ccc(N(c5ccccc5)c5ccc(-c6ccccc6)cc5)cc4)c(c31)O2 ; Cc1cccc2c1N1c3ccccc3C(C)(C)c3ccc(-c4ccc(N(c5ccc(-c6ccccc6)cc5)c5ccc(-c6ccccc6)cc5)cc4)c(c31)O2]

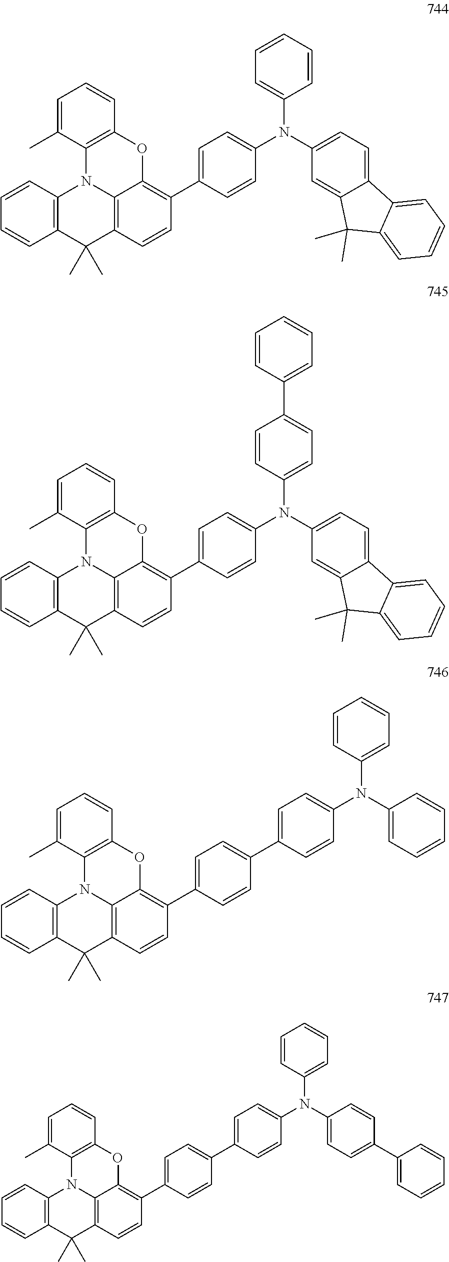 [SMILES: Cc1cccc2c1N1c3ccccc3C(C)(C)c3ccc(-c4ccc(N(c5ccccc5)c5ccc6c(c5)C(C)(C)c5ccccc5-6)cc4)c(c31)O2 ; Cc1cccc2c1N1c3ccccc3C(C)(C)c3ccc(-c4ccc(N(c5ccc(-c6ccccc6)cc5)c5ccc6c(c5)C(C)(C)c5ccccc5-6)cc4)c(c31)O2 ; Cc1cccc2c1N1c3ccccc3C(C)(C)c3ccc(-c4ccc(-c5ccc(N(c6ccccc6)c6ccccc6)cc5)cc4)c(c31)O2 ; Cc1cccc2c1N1c3ccccc3C(C)(C)c3ccc(-c4ccc(-c5ccc(N(c6ccccc6)c6ccc(-c7ccccc7)cc6)cc5)cc4)c(c31)O2]

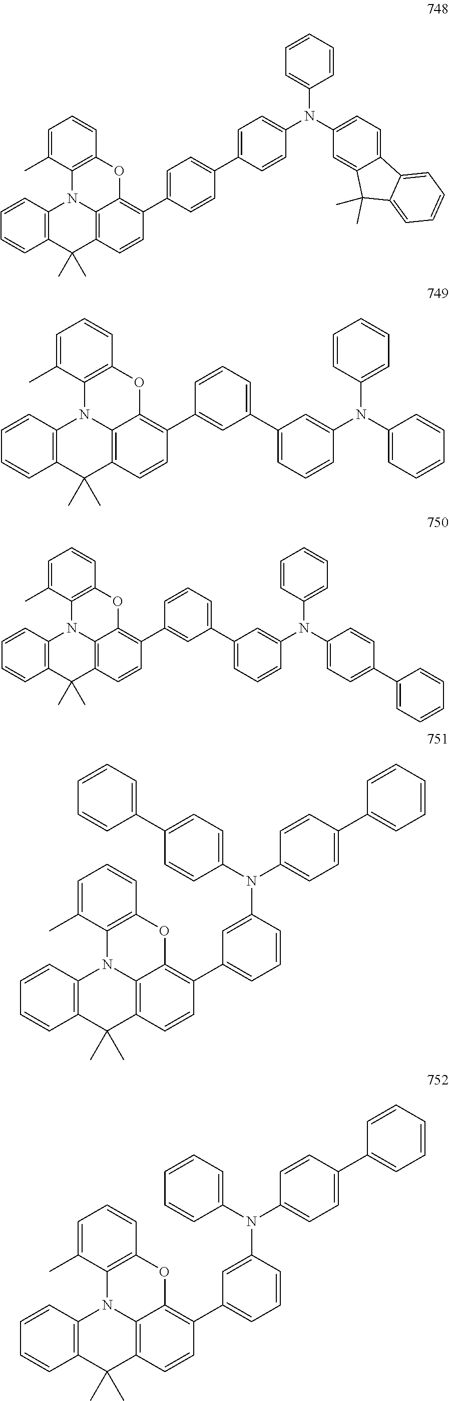

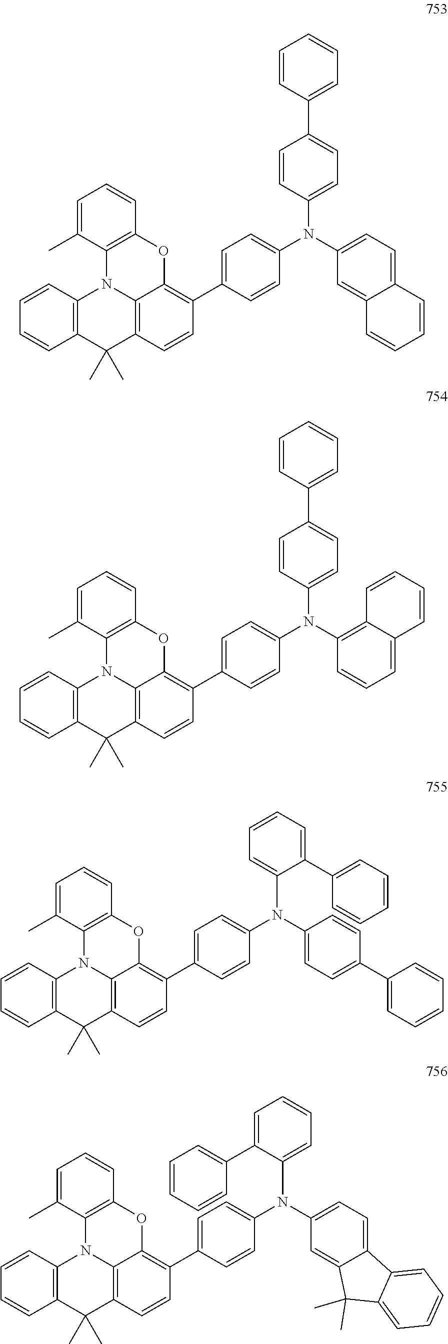 [SMILES: Cc1cccc2c1N1c3ccccc3C(C)(C)c3ccc(-c4ccc(N(c5ccc(-c6ccccc6)cc5)c5ccc6ccccc6c5)cc4)c(c31)O2 ; Cc1cccc2c1N1c3ccccc3C(C)(C)c3ccc(-c4ccc(N(c5ccc(-c6ccccc6)cc5)c5cccc6ccccc56)cc4)c(c31)O2 ; Cc1cccc2c1N1c3ccccc3C(C)(C)c3ccc(-c4ccc(N(c5ccc(-c6ccccc6)cc5)c5ccccc5-c5ccccc5)cc4)c(c31)O2 ; Cc1cccc2c1N1c3ccccc3C(C)(C)c3ccc(-c4ccc(N(c5ccc6c(c5)C(C)(C)c5ccccc5-6)c5ccccc5-c5ccccc5)cc4)c(c31)O2]

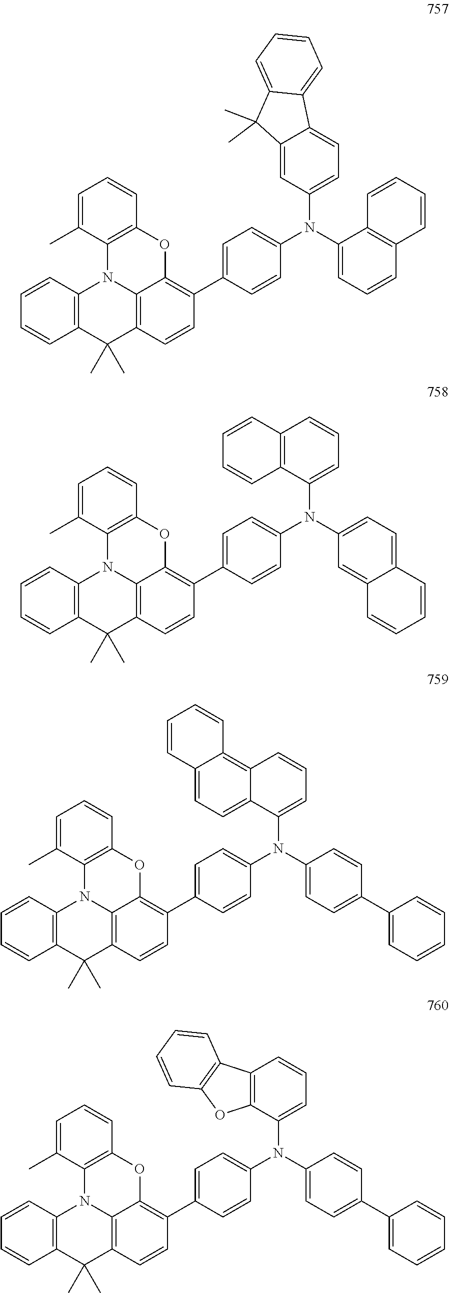 [SMILES: Cc1cccc2c1N1c3ccccc3C(C)(C)c3ccc(-c4ccc(N(c5ccc6c(c5)C(C)(C)c5ccccc5-6)c5cccc6ccccc56)cc4)c(c31)O2 ; Cc1cccc2c1N1c3ccccc3C(C)(C)c3ccc(-c4ccc(N(c5ccc6ccccc6c5)c5cccc6ccccc56)cc4)c(c31)O2 ; Cc1cccc2c1N1c3ccccc3C(C)(C)c3ccc(-c4ccc(N(c5ccc(-c6ccccc6)cc5)c5cccc6c5ccc5ccccc56)cc4)c(c31)O2 ; Cc1cccc2c1N1c3ccccc3C(C)(C)c3ccc(-c4ccc(N(c5ccc(-c6ccccc6)cc5)c5cccc6c5oc5ccccc56)cc4)c(c31)O2]

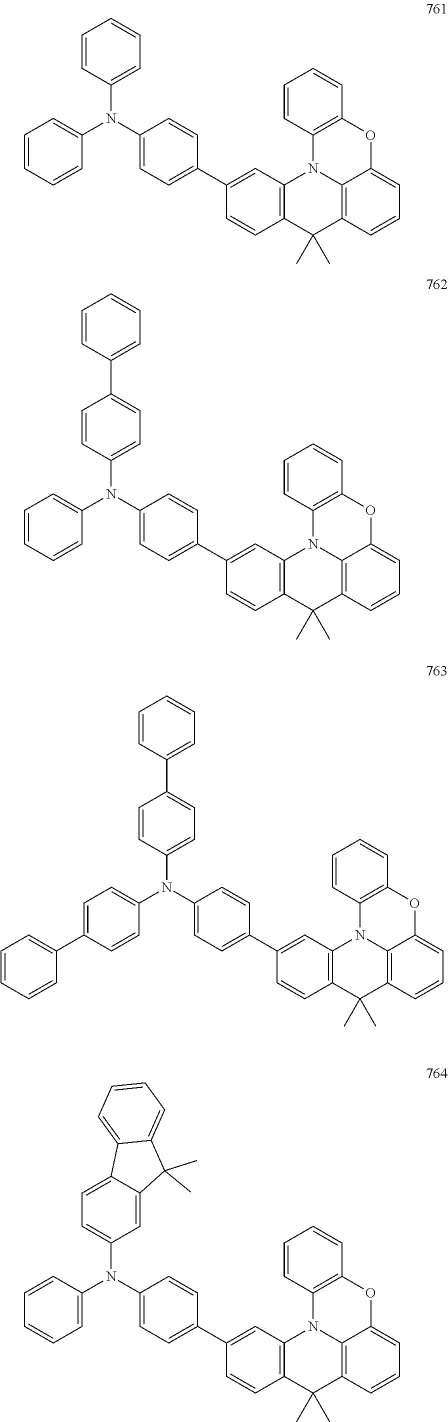 [SMILES: CC1(C)c2ccc(-c3ccc(N(c4ccccc4)c4ccccc4)cc3)cc2N2c3ccccc3Oc3cccc1c32 ; CC1(C)c2ccc(-c3ccc(N(c4ccccc4)c4ccc(-c5ccccc5)cc4)cc3)cc2N2c3ccccc3Oc3cccc1c32 ; CC1(C)c2ccc(-c3ccc(N(c4ccc(-c5ccccc5)cc4)c4ccc(-c5ccccc5)cc4)cc3)cc2N2c3ccccc3Oc3cccc1c32 ; CC1(C)c2ccccc2-c2ccc(N(c3ccccc3)c3ccc(-c4ccc5c(c4)N4c6ccccc6Oc6cccc(c64)C5(C)C)cc3)cc21]

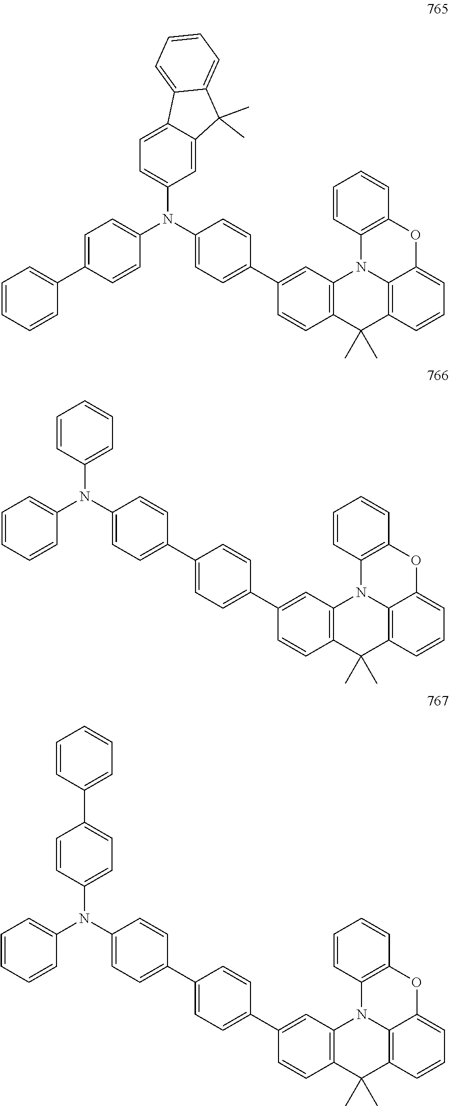 [SMILES: CC1(C)c2ccccc2-c2ccc(N(c3ccc(-c4ccccc4)cc3)c3ccc(-c4ccc5c(c4)N4c6ccccc6Oc6cccc(c64)C5(C)C)cc3)cc21 ; CC1(C)c2ccc(-c3ccc(-c4ccc(N(c5ccccc5)c5ccccc5)cc4)cc3)cc2N2c3ccccc3Oc3cccc1c32 ; CC1(C)c2ccc(-c3ccc(-c4ccc(N(c5ccccc5)c5ccc(-c6ccccc6)cc5)cc4)cc3)cc2N2c3ccccc3Oc3cccc1c32]

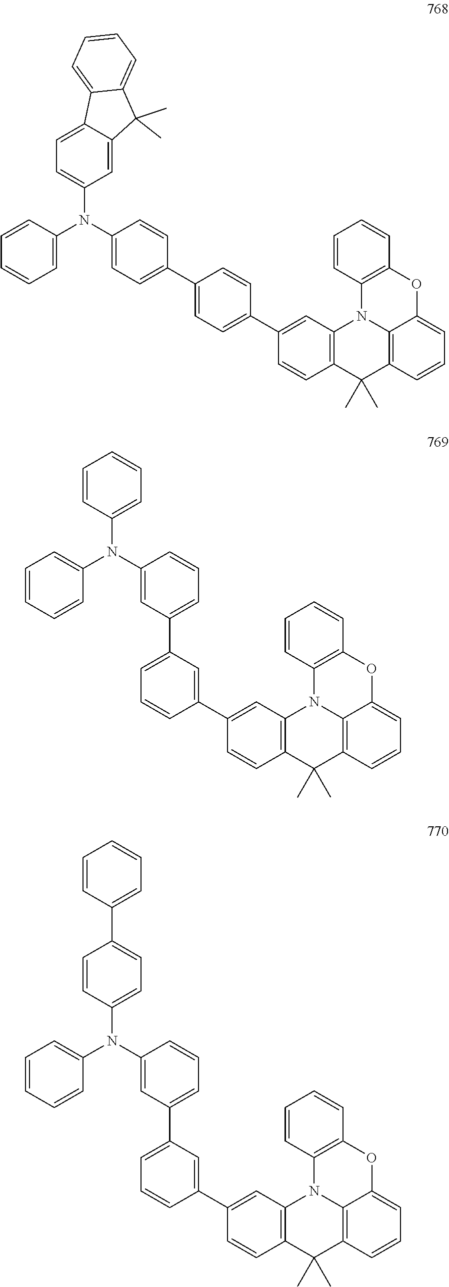 [SMILES: CC1(C)c2ccccc2-c2ccc(N(c3ccccc3)c3ccc(-c4ccc(-c5ccc6c(c5)N5c7ccccc7Oc7cccc(c75)C6(C)C)cc4)cc3)cc21 ; CC1(C)c2ccc(-c3cccc(-c4cccc(N(c5ccccc5)c5ccccc5)c4)c3)cc2N2c3ccccc3Oc3cccc1c32 ; CC1(C)c2ccc(-c3cccc(-c4cccc(N(c5ccccc5)c5ccc(-c6ccccc6)cc5)c4)c3)cc2N2c3ccccc3Oc3cccc1c32]

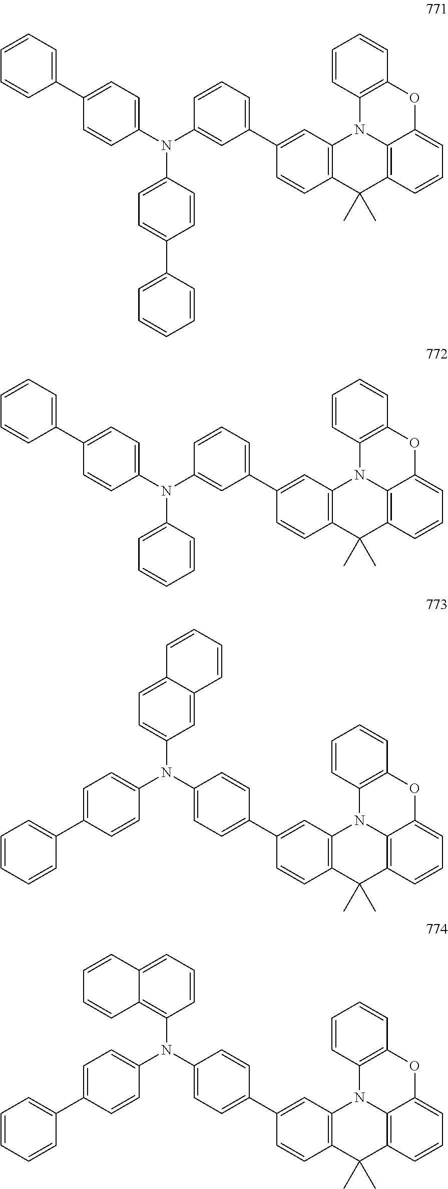 [SMILES: CC1(C)c2ccc(-c3cccc(N(c4ccc(-c5ccccc5)cc4)c4ccc(-c5ccccc5)cc4)c3)cc2N2c3ccccc3Oc3cccc1c32 ; CC1(C)c2ccc(-c3cccc(N(c4ccccc4)c4ccc(-c5ccccc5)cc4)c3)cc2N2c3ccccc3Oc3cccc1c32 ; CC1(C)c2ccc(-c3ccc(N(c4ccc(-c5ccccc5)cc4)c4ccc5ccccc5c4)cc3)cc2N2c3ccccc3Oc3cccc1c32 ; CC1(C)c2ccc(-c3ccc(N(c4ccc(-c5ccccc5)cc4)c4cccc5ccccc45)cc3)cc2N2c3ccccc3Oc3cccc1c32]

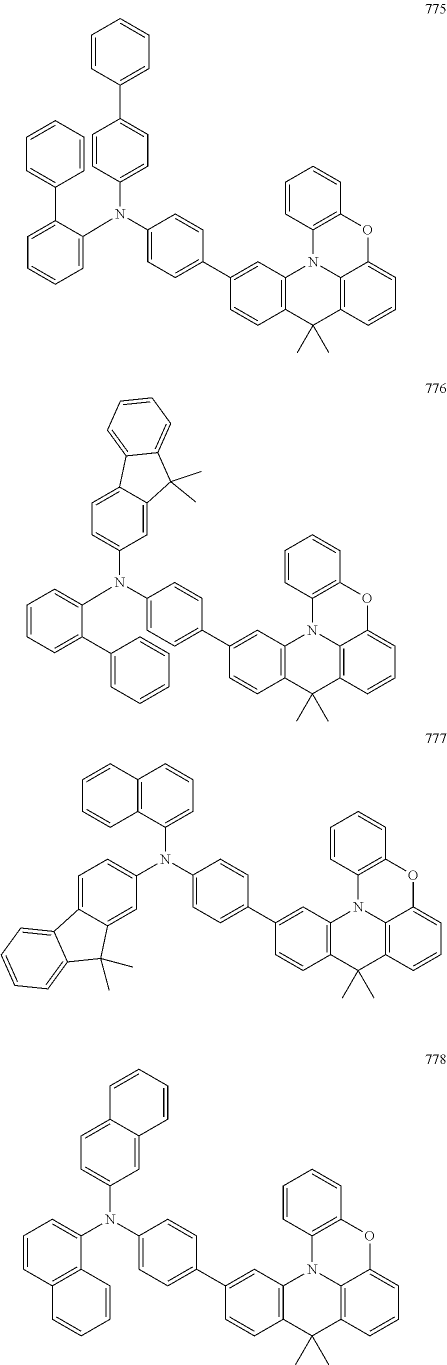 [SMILES: CC1(C)c2ccc(-c3ccc(N(c4ccc(-c5ccccc5)cc4)c4ccccc4-c4ccccc4)cc3)cc2N2c3ccccc3Oc3cccc1c32 ; CC1(C)c2ccccc2-c2ccc(N(c3ccc(-c4ccc5c(c4)N4c6ccccc6Oc6cccc(c64)C5(C)C)cc3)c3ccccc3-c3ccccc3)cc21 ; CC1(C)c2ccccc2-c2ccc(N(c3ccc(-c4ccc5c(c4)N4c6ccccc6Oc6cccc(c64)C5(C)C)cc3)c3cccc4ccccc34)cc21 ; CC1(C)c2ccc(-c3ccc(N(c4ccc5ccccc5c4)c4cccc5ccccc45)cc3)cc2N2c3ccccc3Oc3cccc1c32]

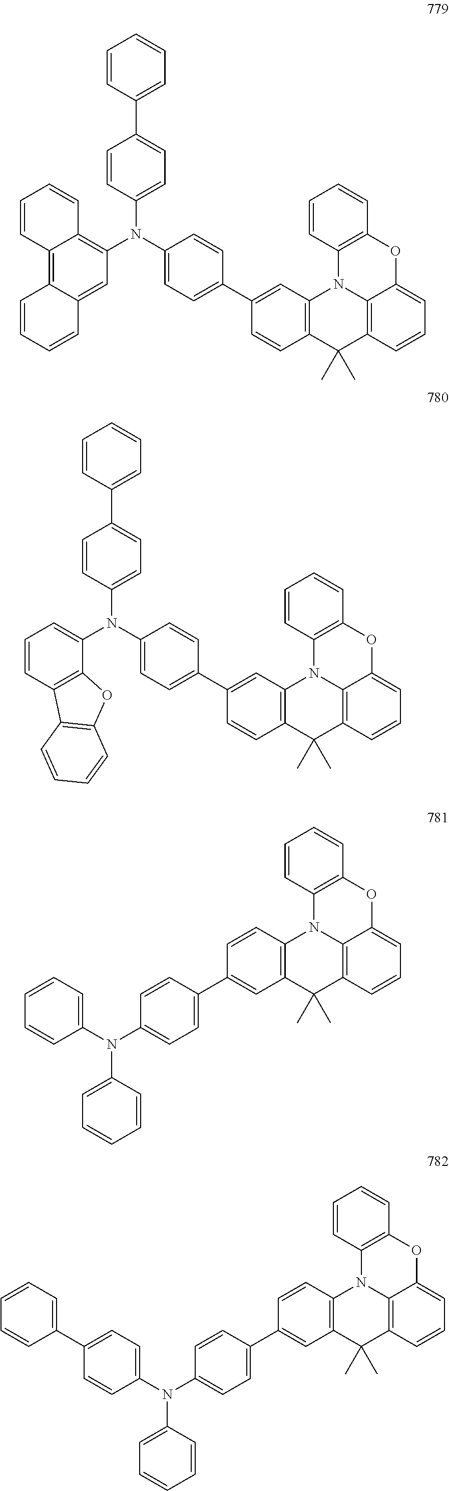 [SMILES: CC1(C)c2ccc(-c3ccc(N(c4ccc(-c5ccccc5)cc4)c4cc5ccccc5c5ccccc45)cc3)cc2N2c3ccccc3Oc3cccc1c32 ; CC1(C)c2ccc(-c3ccc(N(c4ccc(-c5ccccc5)cc4)c4cccc5c4oc4ccccc45)cc3)cc2N2c3ccccc3Oc3cccc1c32 ; CC1(C)c2cc(-c3ccc(N(c4ccccc4)c4ccccc4)cc3)ccc2N2c3ccccc3Oc3cccc1c32 ; CC1(C)c2cc(-c3ccc(N(c4ccccc4)c4ccc(-c5ccccc5)cc4)cc3)ccc2N2c3ccccc3Oc3cccc1c32]

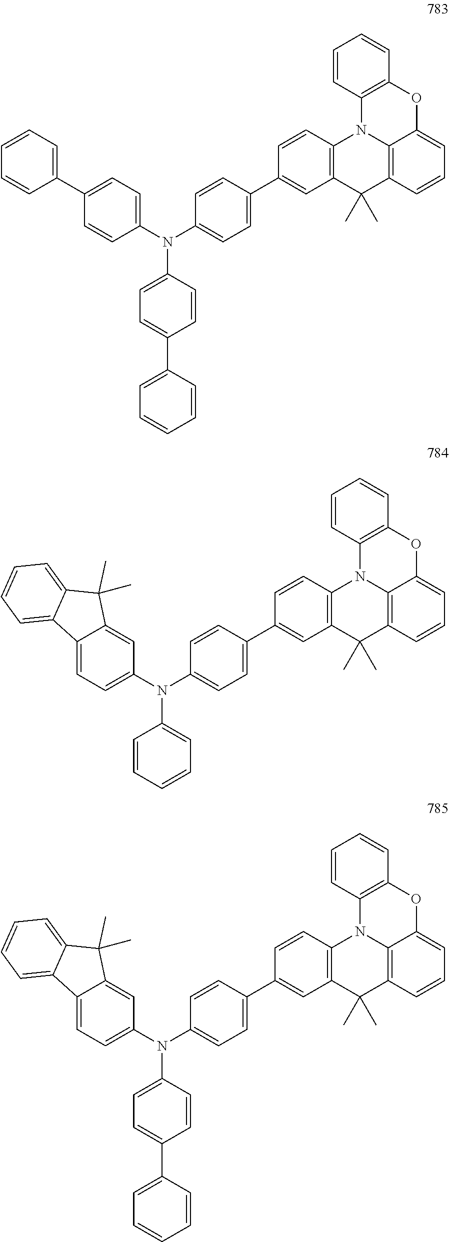 [SMILES: CC1(C)c2cc(-c3ccc(N(c4ccc(-c5ccccc5)cc4)c4ccc(-c5ccccc5)cc4)cc3)ccc2N2c3ccccc3Oc3cccc1c32 ; CC1(C)c2ccccc2-c2ccc(N(c3ccccc3)c3ccc(-c4ccc5c(c4)C(C)(C)c4cccc6c4N5c4ccccc4O6)cc3)cc21 ; CC1(C)c2ccccc2-c2ccc(N(c3ccc(-c4ccccc4)cc3)c3ccc(-c4ccc5c(c4)C(C)(C)c4cccc6c4N5c4ccccc4O6)cc3)cc21]

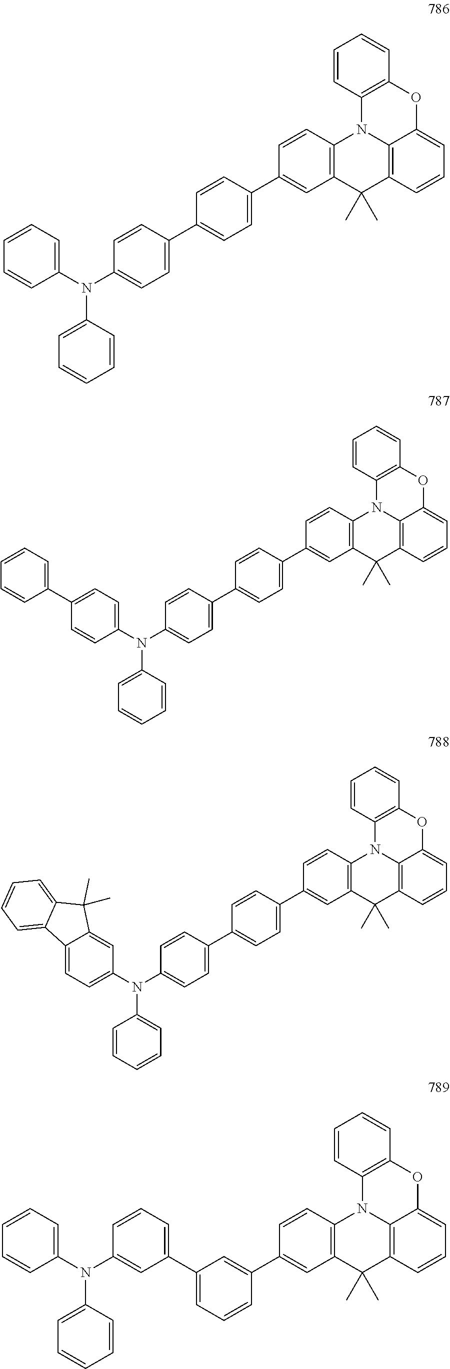 [SMILES: CC1(C)c2cc(-c3ccc(-c4ccc(N(c5ccccc5)c5ccccc5)cc4)cc3)ccc2N2c3ccccc3Oc3cccc1c32 ; CC1(C)c2cc(-c3ccc(-c4ccc(N(c5ccccc5)c5ccc(-c6ccccc6)cc5)cc4)cc3)ccc2N2c3ccccc3Oc3cccc1c32 ; CC1(C)c2ccccc2-c2ccc(N(c3ccccc3)c3ccc(-c4ccc(-c5ccc6c(c5)C(C)(C)c5cccc7c5N6c5ccccc5O7)cc4)cc3)cc21 ; CC1(C)c2cc(-c3cccc(-c4cccc(N(c5ccccc5)c5ccccc5)c4)c3)ccc2N2c3ccccc3Oc3cccc1c32]

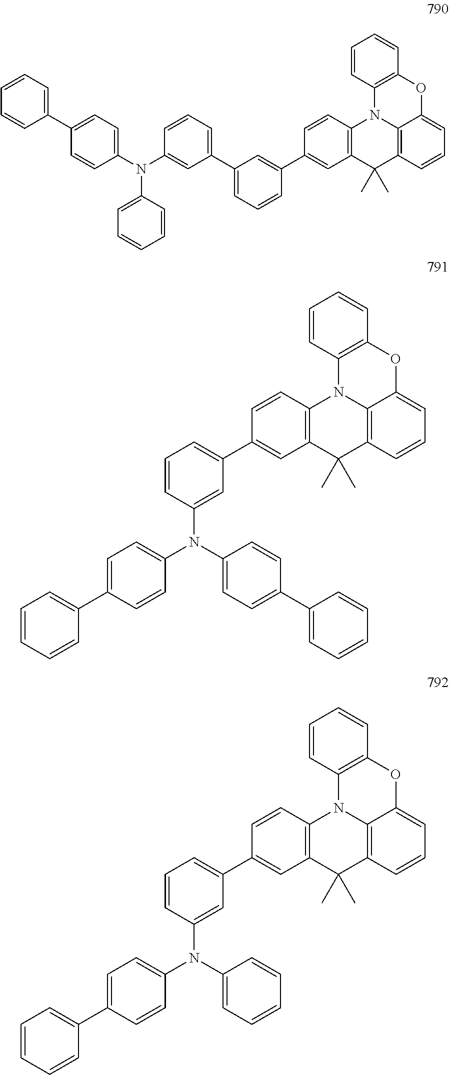 [SMILES: CC1(C)c2cc(-c3cccc(-c4cccc(N(c5ccccc5)c5ccc(-c6ccccc6)cc5)c4)c3)ccc2N2c3ccccc3Oc3cccc1c32 ; CC1(C)c2cc(-c3cccc(N(c4ccc(-c5ccccc5)cc4)c4ccc(-c5ccccc5)cc4)c3)ccc2N2c3ccccc3Oc3cccc1c32 ; CC1(C)c2cc(-c3cccc(N(c4ccccc4)c4ccc(-c5ccccc5)cc4)c3)ccc2N2c3ccccc3Oc3cccc1c32]

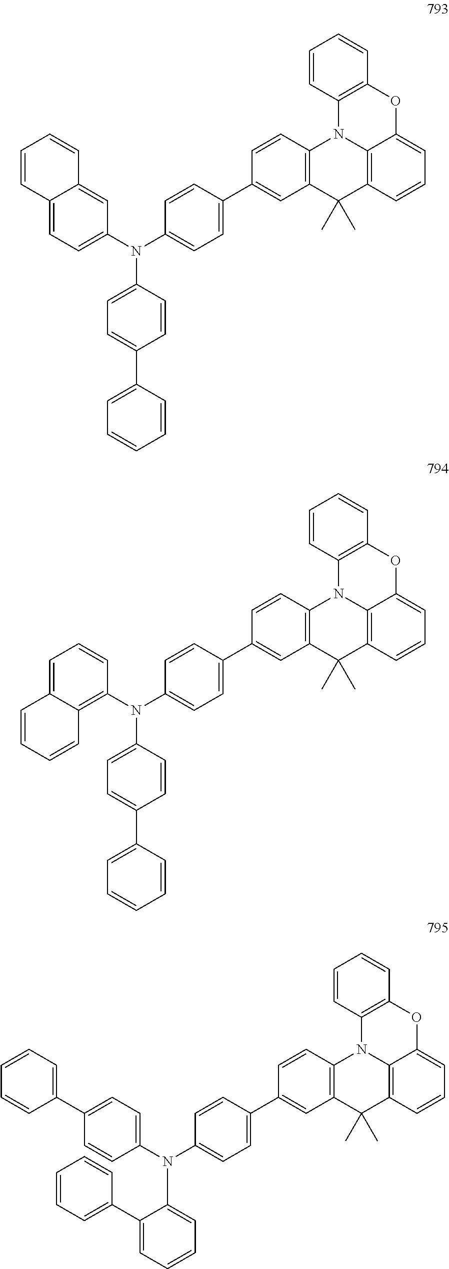 [SMILES: CC1(C)c2cc(-c3ccc(N(c4ccc(-c5ccccc5)cc4)c4ccc5ccccc5c4)cc3)ccc2N2c3ccccc3Oc3cccc1c32 ; CC1(C)c2cc(-c3ccc(N(c4ccc(-c5ccccc5)cc4)c4cccc5ccccc45)cc3)ccc2N2c3ccccc3Oc3cccc1c32 ; CC1(C)c2cc(-c3ccc(N(c4ccc(-c5ccccc5)cc4)c4ccccc4-c4ccccc4)cc3)ccc2N2c3ccccc3Oc3cccc1c32]

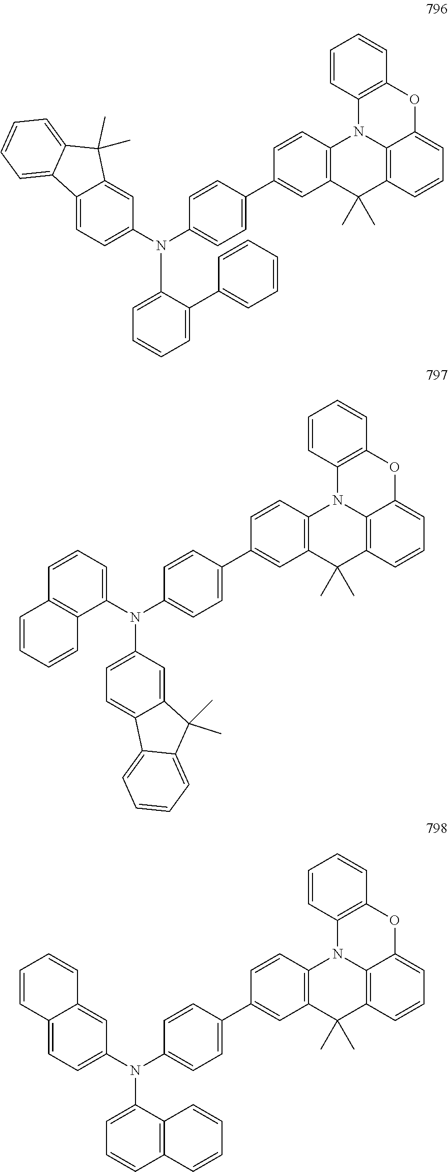 [SMILES: CC1(C)c2ccccc2-c2ccc(N(c3ccc(-c4ccc5c(c4)C(C)(C)c4cccc6c4N5c4ccccc4O6)cc3)c3ccccc3-c3ccccc3)cc21 ; CC1(C)c2ccccc2-c2ccc(N(c3ccc(-c4ccc5c(c4)C(C)(C)c4cccc6c4N5c4ccccc4O6)cc3)c3cccc4ccccc34)cc21 ; CC1(C)c2cc(-c3ccc(N(c4ccc5ccccc5c4)c4cccc5ccccc45)cc3)ccc2N2c3ccccc3Oc3cccc1c32]

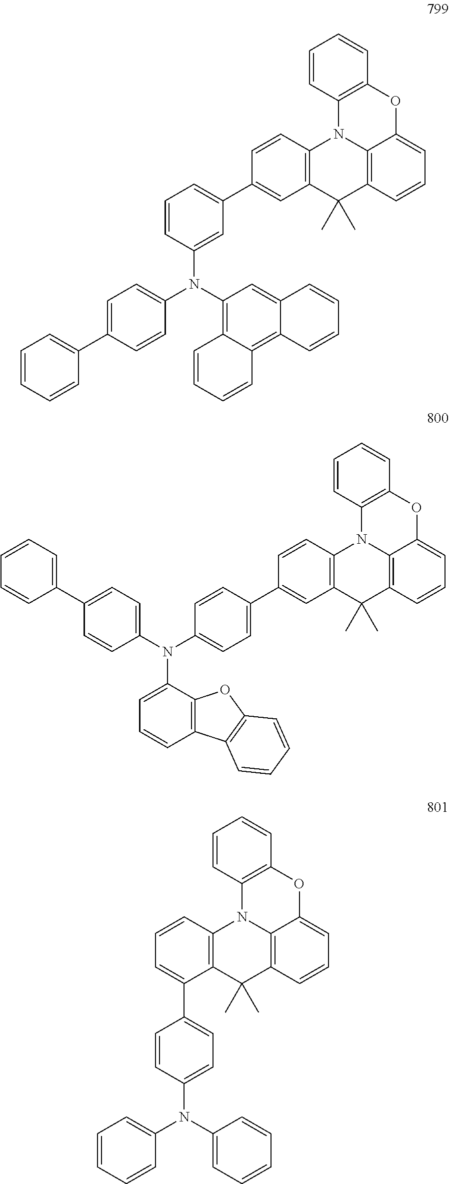

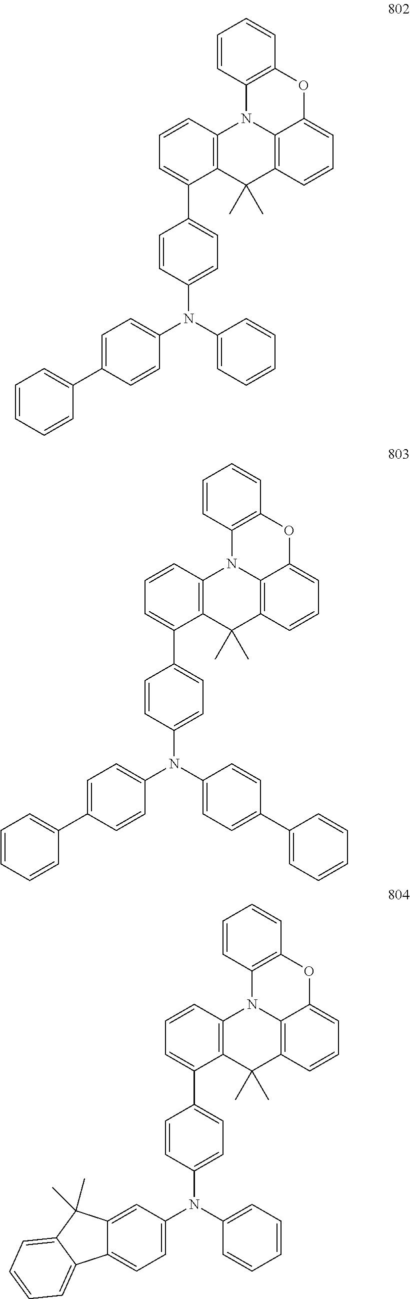 [SMILES: CC1(C)c2cccc3c2N(c2ccccc2O3)c2cccc(-c3ccc(N(c4ccccc4)c4ccc(-c5ccccc5)cc4)cc3)c21 ; CC1(C)c2cccc3c2N(c2ccccc2O3)c2cccc(-c3ccc(N(c4ccc(-c5ccccc5)cc4)c4ccc(-c5ccccc5)cc4)cc3)c21 ; CC1(C)c2ccccc2-c2ccc(N(c3ccccc3)c3ccc(-c4cccc5c4C(C)(C)c4cccc6c4N5c4ccccc4O6)cc3)cc21]

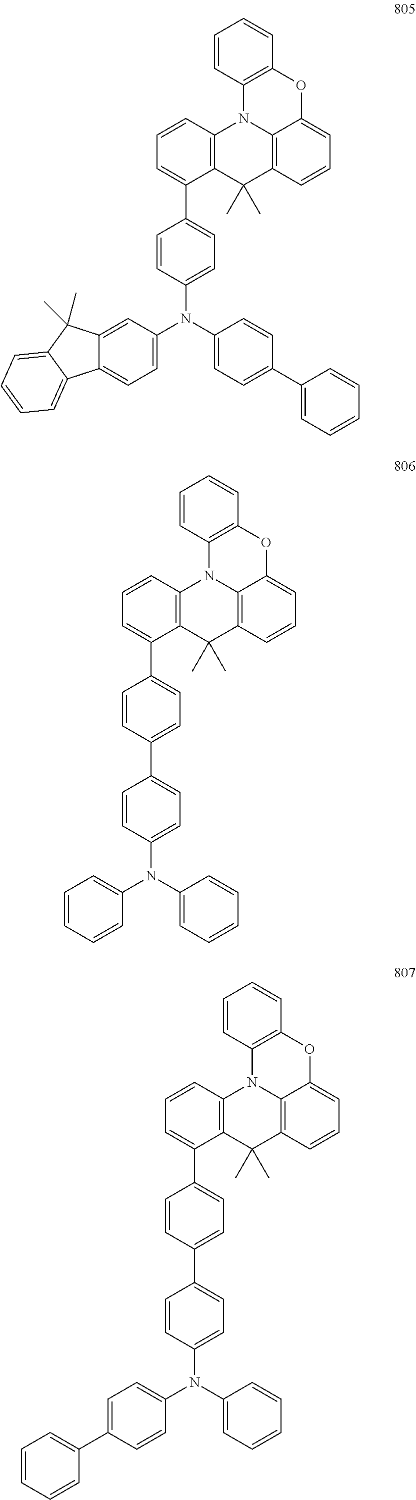 [SMILES: CC1(C)c2ccccc2-c2ccc(N(c3ccc(-c4ccccc4)cc3)c3ccc(-c4cccc5c4C(C)(C)c4cccc6c4N5c4ccccc4O6)cc3)cc21 ; CC1(C)c2cccc3c2N(c2ccccc2O3)c2cccc(-c3ccc(-c4ccc(N(c5ccccc5)c5ccccc5)cc4)cc3)c21 ; CC1(C)c2cccc3c2N(c2ccccc2O3)c2cccc(-c3ccc(-c4ccc(N(c5ccccc5)c5ccc(-c6ccccc6)cc5)cc4)cc3)c21]

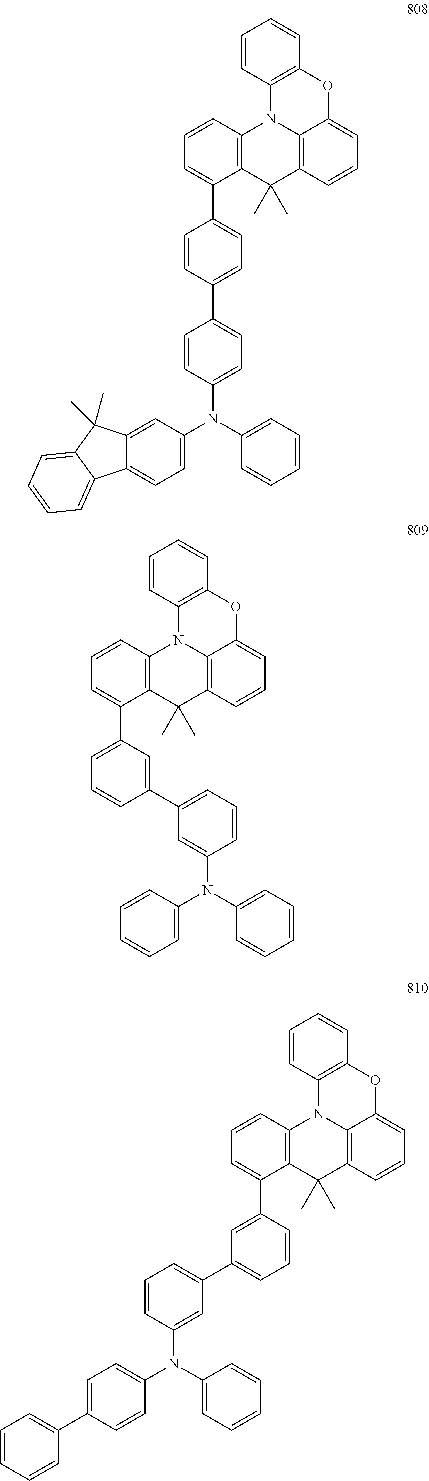 [SMILES: CC1(C)c2ccccc2-c2ccc(N(c3ccccc3)c3ccc(-c4ccc(-c5cccc6c5C(C)(C)c5cccc7c5N6c5ccccc5O7)cc4)cc3)cc21 ; CC1(C)c2cccc3c2N(c2ccccc2O3)c2cccc(-c3cccc(-c4cccc(N(c5ccccc5)c5ccccc5)c4)c3)c21 ; CC1(C)c2cccc3c2N(c2ccccc2O3)c2cccc(-c3cccc(-c4cccc(N(c5ccccc5)c5ccc(-c6ccccc6)cc5)c4)c3)c21]

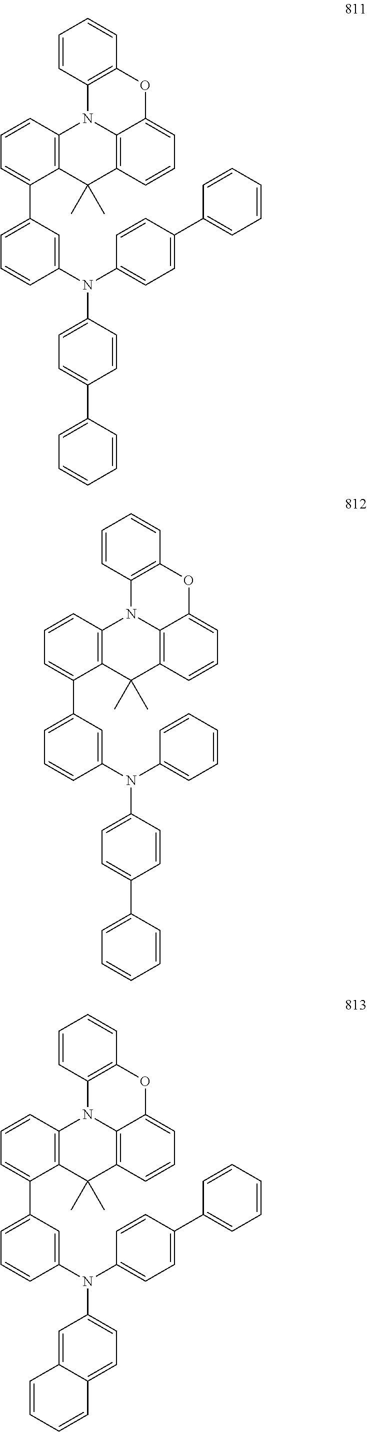 [SMILES: CC1(C)c2cccc3c2N(c2ccccc2O3)c2cccc(-c3cccc(N(c4ccc(-c5ccccc5)cc4)c4ccc(-c5ccccc5)cc4)c3)c21 ; CC1(C)c2cccc3c2N(c2ccccc2O3)c2cccc(-c3cccc(N(c4ccccc4)c4ccc(-c5ccccc5)cc4)c3)c21 ; CC1(C)c2cccc3c2N(c2ccccc2O3)c2cccc(-c3cccc(N(c4ccc(-c5ccccc5)cc4)c4ccc5ccccc5c4)c3)c21]

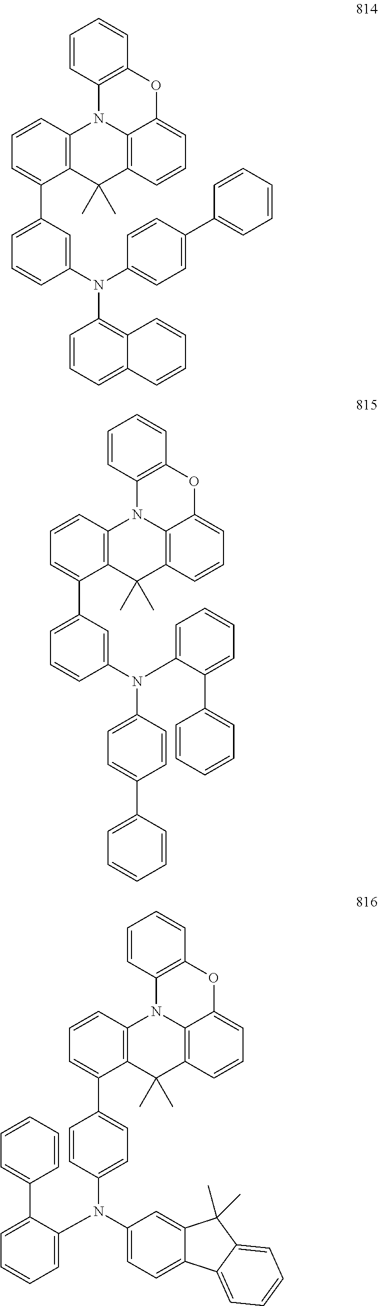

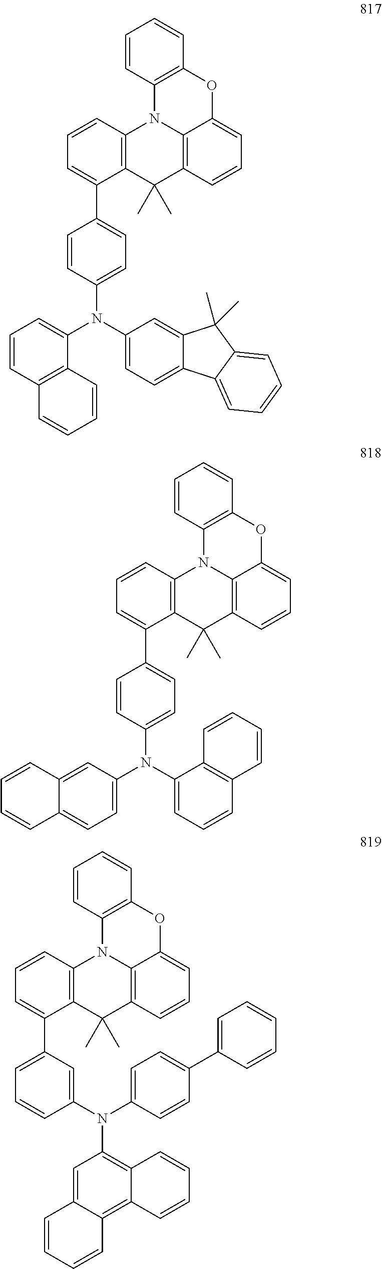

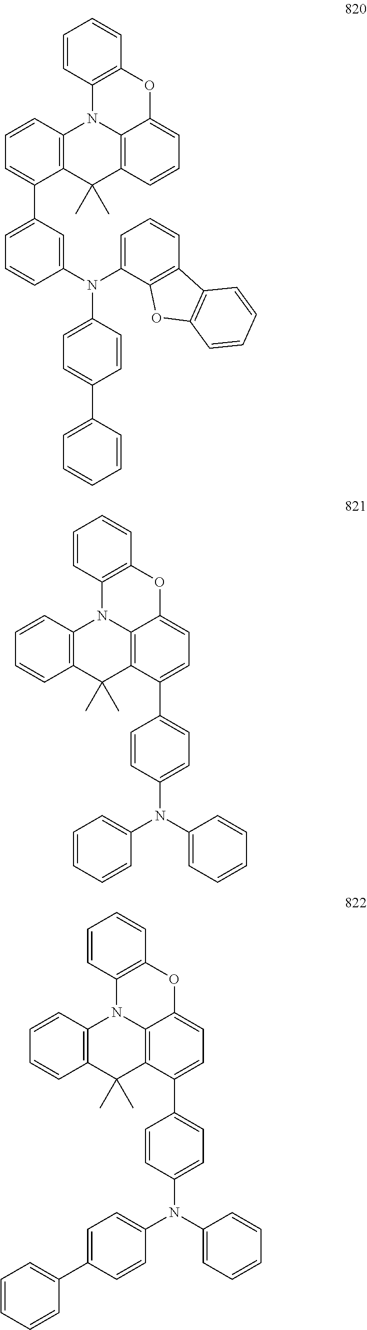

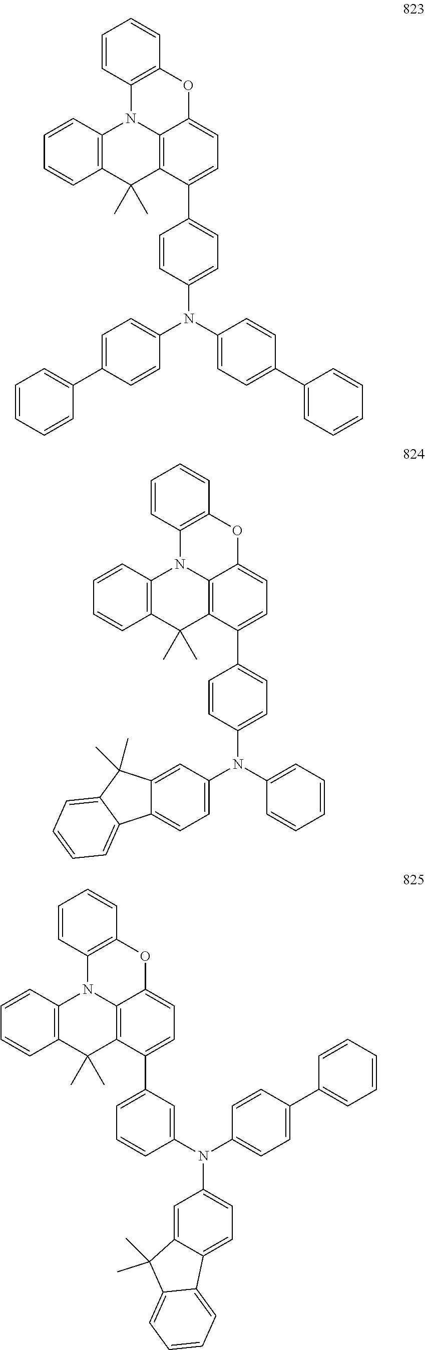

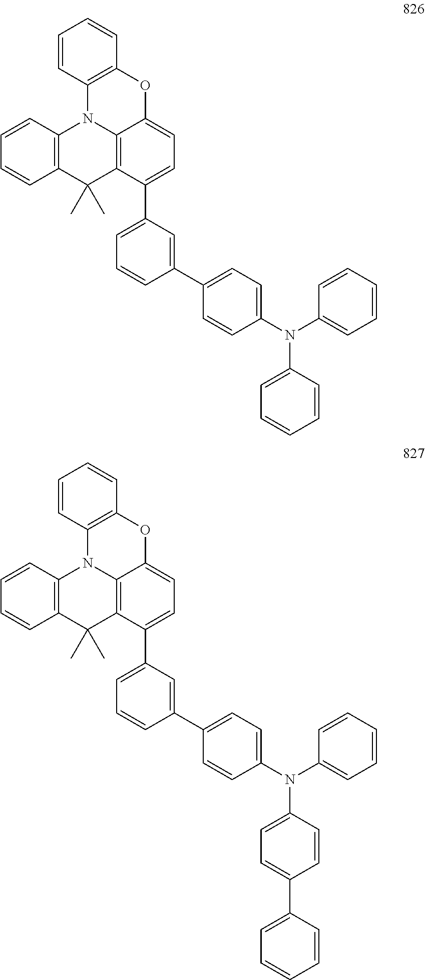 [SMILES: CC1(C)c2ccccc2N2c3ccccc3Oc3ccc(-c4cccc(-c5ccc(N(c6ccccc6)c6ccccc6)cc5)c4)c1c32 ; CC1(C)c2ccccc2N2c3ccccc3Oc3ccc(-c4cccc(-c5ccc(N(c6ccccc6)c6ccc(-c7ccccc7)cc6)cc5)c4)c1c32]

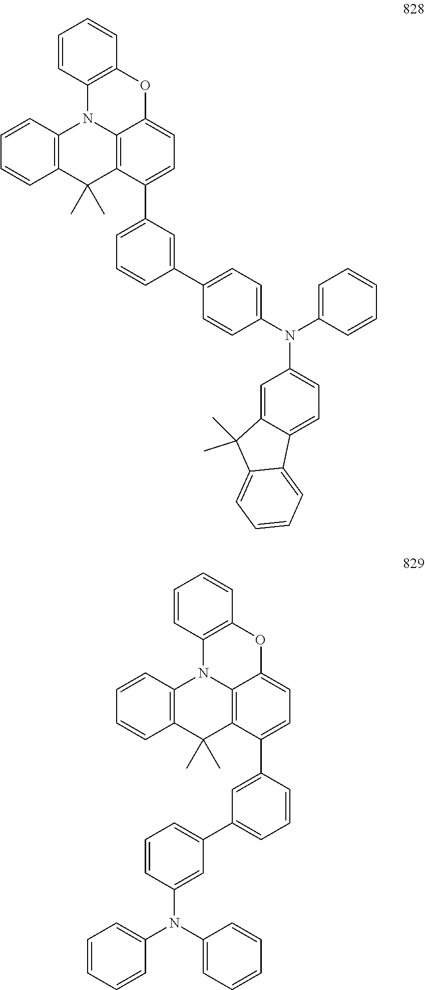 [SMILES: CC1(C)c2ccccc2-c2ccc(N(c3ccccc3)c3ccc(-c4cccc(-c5ccc6c7c5C(C)(C)c5ccccc5N7c5ccccc5O6)c4)cc3)cc21 ; CC1(C)c2ccccc2N2c3ccccc3Oc3ccc(-c4cccc(-c5cccc(N(c6ccccc6)c6ccccc6)c5)c4)c1c32]

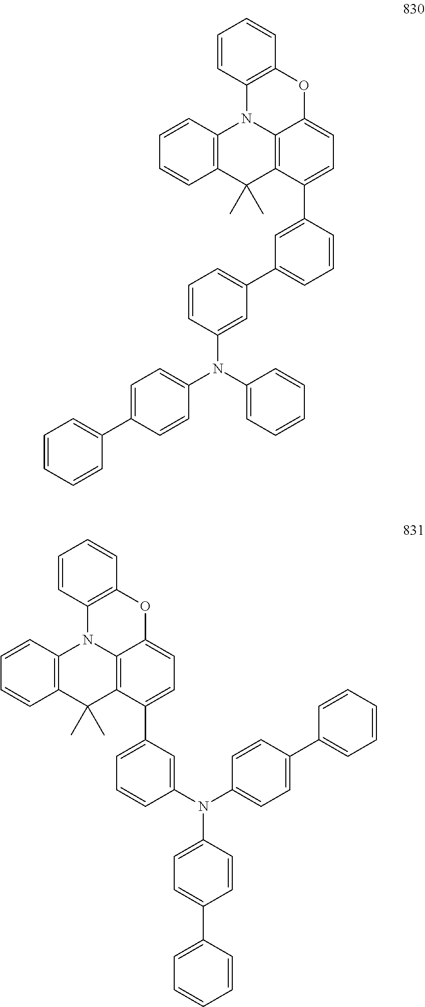 [SMILES: CC1(C)c2ccccc2N2c3ccccc3Oc3ccc(-c4cccc(-c5cccc(N(c6ccccc6)c6ccc(-c7ccccc7)cc6)c5)c4)c1c32 ; CC1(C)c2ccccc2N2c3ccccc3Oc3ccc(-c4cccc(N(c5ccc(-c6ccccc6)cc5)c5ccc(-c6ccccc6)cc5)c4)c1c32]

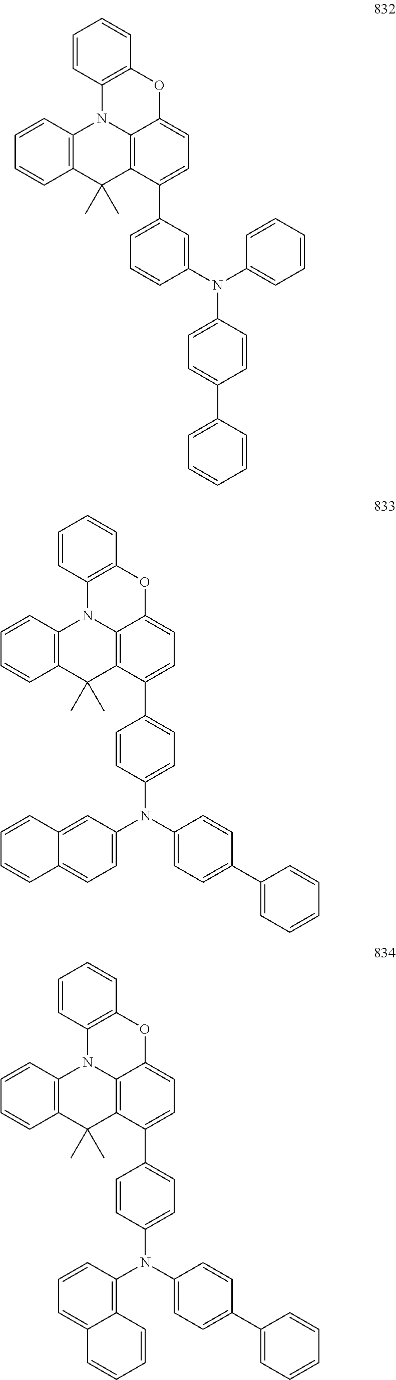 [SMILES: CC1(C)c2ccccc2N2c3ccccc3Oc3ccc(-c4cccc(N(c5ccccc5)c5ccc(-c6ccccc6)cc5)c4)c1c32 ; CC1(C)c2ccccc2N2c3ccccc3Oc3ccc(-c4ccc(N(c5ccc(-c6ccccc6)cc5)c5ccc6ccccc6c5)cc4)c1c32 ; CC1(C)c2ccccc2N2c3ccccc3Oc3ccc(-c4ccc(N(c5ccc(-c6ccccc6)cc5)c5cccc6ccccc56)cc4)c1c32]

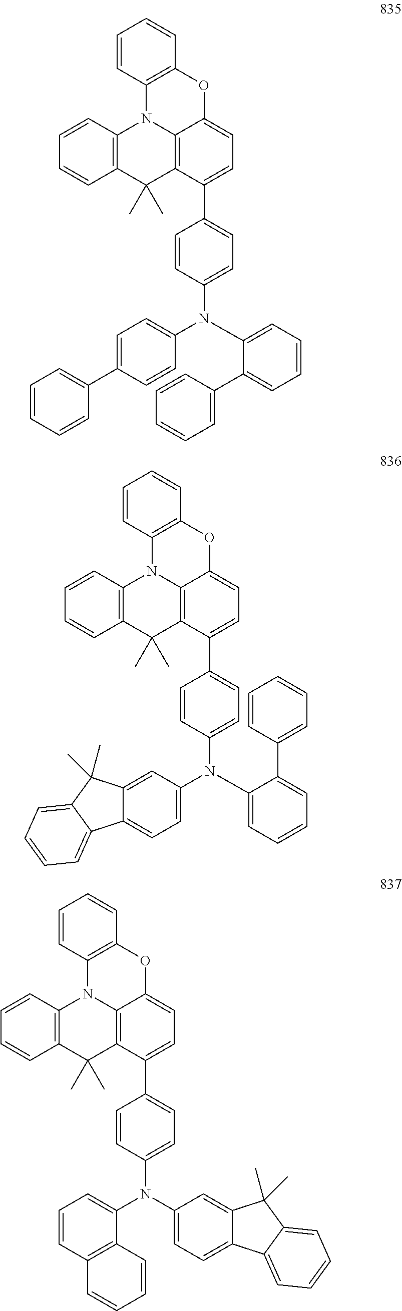

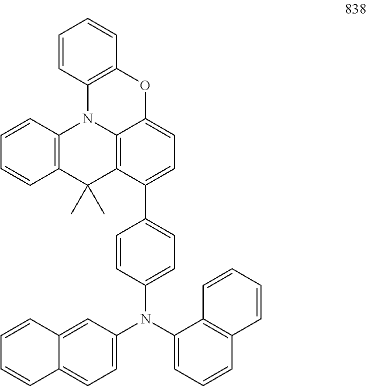 [SMILES: CC1(C)c2ccccc2N2c3ccccc3Oc3ccc(-c4ccc(N(c5ccc6ccccc6c5)c5cccc6ccccc56)cc4)c1c32]

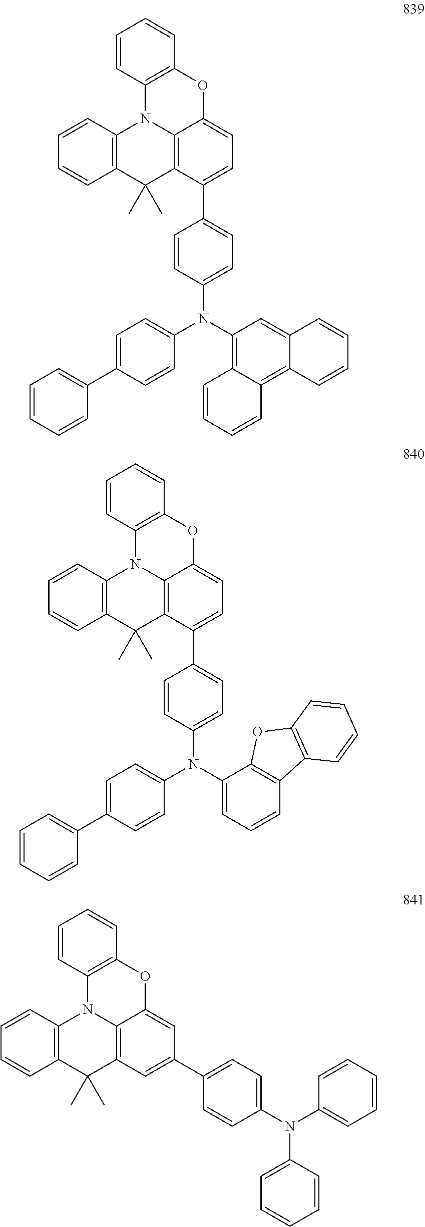

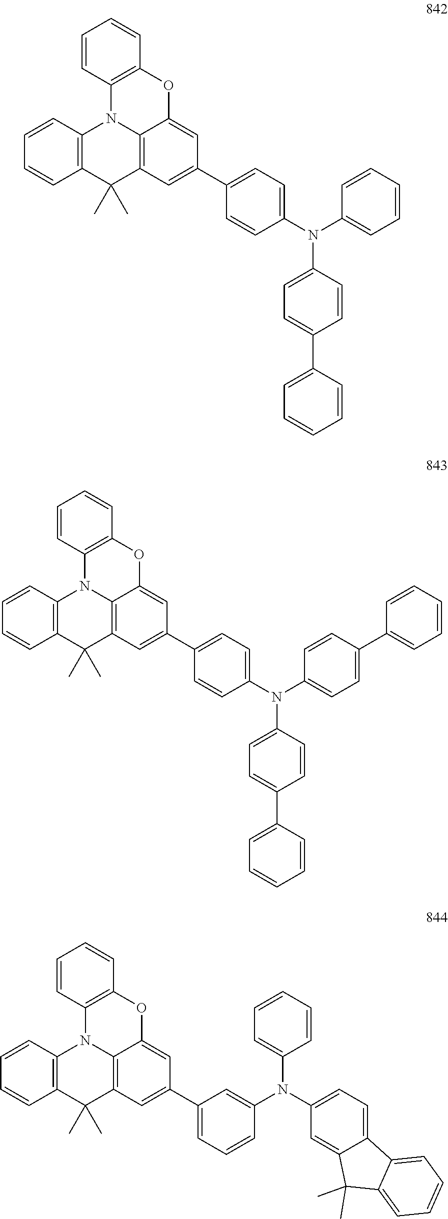 [SMILES: CC1(C)c2ccccc2N2c3ccccc3Oc3cc(-c4ccc(N(c5ccccc5)c5ccc(-c6ccccc6)cc5)cc4)cc1c32 ; CC1(C)c2ccccc2N2c3ccccc3Oc3cc(-c4ccc(N(c5ccc(-c6ccccc6)cc5)c5ccc(-c6ccccc6)cc5)cc4)cc1c32 ; CC1(C)c2ccccc2-c2ccc(N(c3ccccc3)c3cccc(-c4cc5c6c(c4)C(C)(C)c4ccccc4N6c4ccccc4O5)c3)cc21]

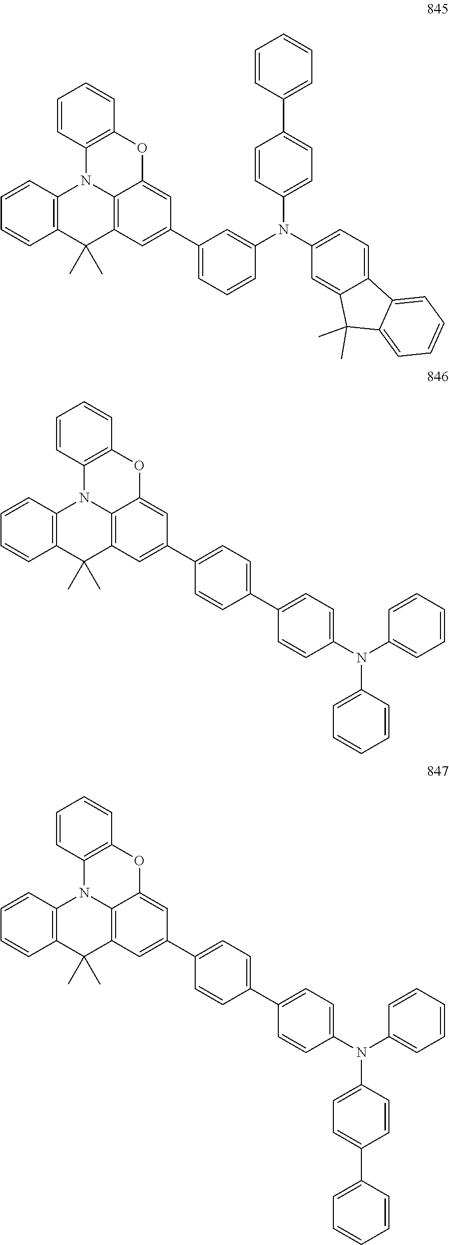 [SMILES: CC1(C)c2ccccc2-c2ccc(N(c3ccc(-c4ccccc4)cc3)c3cccc(-c4cc5c6c(c4)C(C)(C)c4ccccc4N6c4ccccc4O5)c3)cc21 ; CC1(C)c2ccccc2N2c3ccccc3Oc3cc(-c4ccc(-c5ccc(N(c6ccccc6)c6ccccc6)cc5)cc4)cc1c32 ; CC1(C)c2ccccc2N2c3ccccc3Oc3cc(-c4ccc(-c5ccc(N(c6ccccc6)c6ccc(-c7ccccc7)cc6)cc5)cc4)cc1c32]

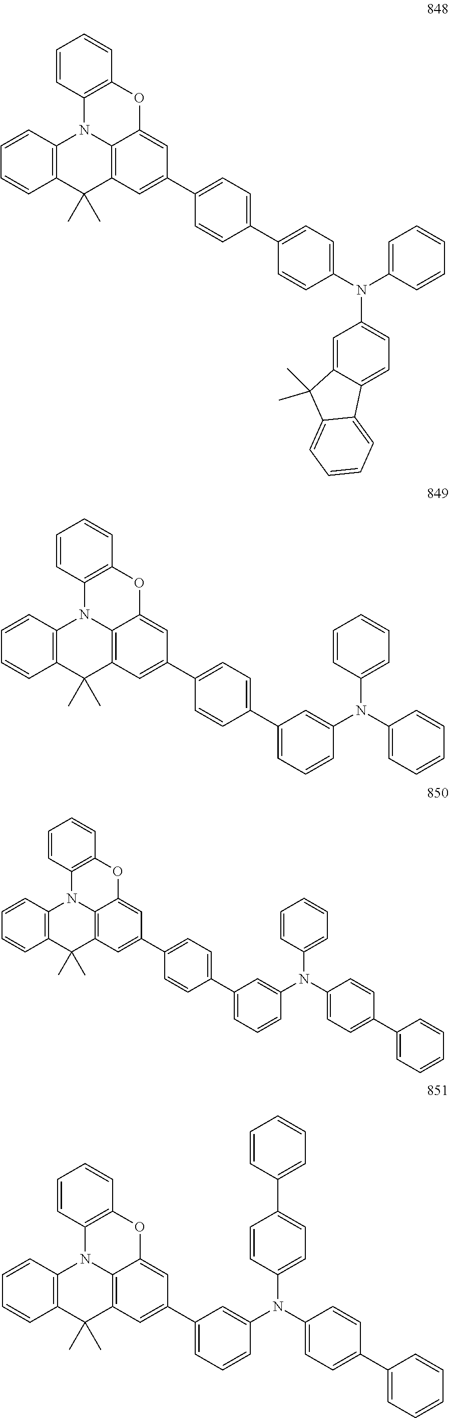 [SMILES: CC1(C)c2ccccc2-c2ccc(N(c3ccccc3)c3ccc(-c4ccc(-c5cc6c7c(c5)C(C)(C)c5ccccc5N7c5ccccc5O6)cc4)cc3)cc21 ; CC1(C)c2ccccc2N2c3ccccc3Oc3cc(-c4ccc(-c5cccc(N(c6ccccc6)c6ccccc6)c5)cc4)cc1c32 ; CC1(C)c2ccccc2N2c3ccccc3Oc3cc(-c4ccc(-c5cccc(N(c6ccccc6)c6ccc(-c7ccccc7)cc6)c5)cc4)cc1c32 ; CC1(C)c2ccccc2N2c3ccccc3Oc3cc(-c4cccc(N(c5ccc(-c6ccccc6)cc5)c5ccc(-c6ccccc6)cc5)c4)cc1c32]

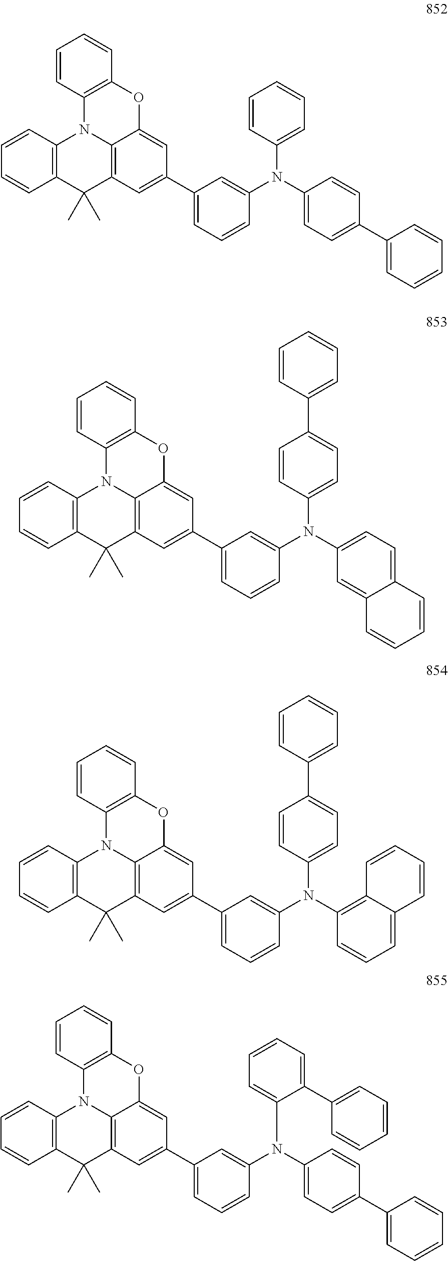 [SMILES: CC1(C)c2ccccc2N2c3ccccc3Oc3cc(-c4cccc(N(c5ccccc5)c5ccc(-c6ccccc6)cc5)c4)cc1c32 ; CC1(C)c2ccccc2N2c3ccccc3Oc3cc(-c4cccc(N(c5ccc(-c6ccccc6)cc5)c5ccc6ccccc6c5)c4)cc1c32 ; CC1(C)c2ccccc2N2c3ccccc3Oc3cc(-c4cccc(N(c5ccc(-c6ccccc6)cc5)c5cccc6ccccc56)c4)cc1c32 ; CC1(C)c2ccccc2N2c3ccccc3Oc3cc(-c4cccc(N(c5ccc(-c6ccccc6)cc5)c5ccccc5-c5ccccc5)c4)cc1c32]

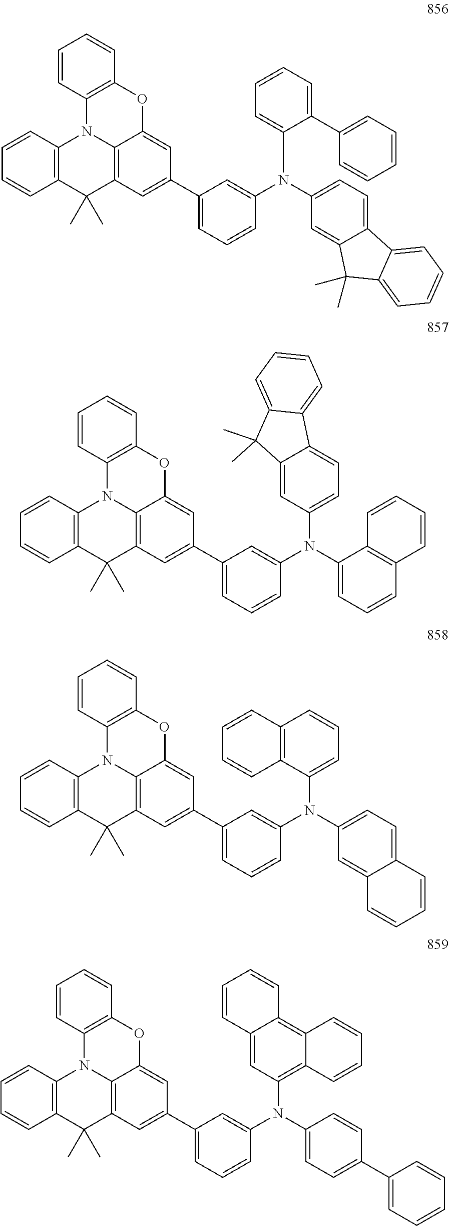 [SMILES: CC1(C)c2ccccc2-c2ccc(N(c3cccc(-c4cc5c6c(c4)C(C)(C)c4ccccc4N6c4ccccc4O5)c3)c3ccccc3-c3ccccc3)cc21 ; CC1(C)c2ccccc2-c2ccc(N(c3cccc(-c4cc5c6c(c4)C(C)(C)c4ccccc4N6c4ccccc4O5)c3)c3cccc4ccccc34)cc21 ; CC1(C)c2ccccc2N2c3ccccc3Oc3cc(-c4cccc(N(c5ccc6ccccc6c5)c5cccc6ccccc56)c4)cc1c32 ; CC1(C)c2ccccc2N2c3ccccc3Oc3cc(-c4cccc(N(c5ccc(-c6ccccc6)cc5)c5cc6ccccc6c6ccccc56)c4)cc1c32]

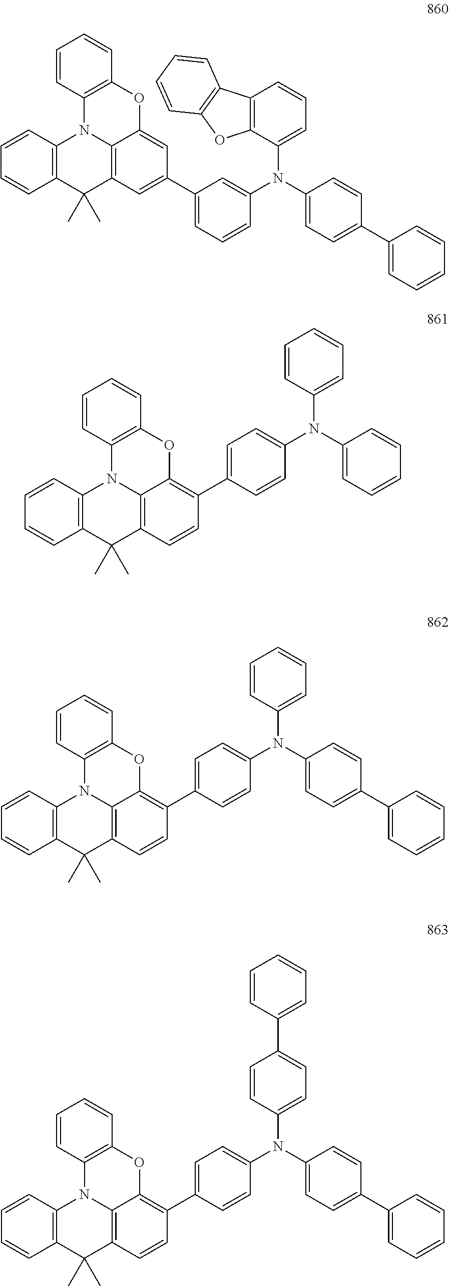 [SMILES: CC1(C)c2ccccc2N2c3ccccc3Oc3cc(-c4cccc(N(c5ccc(-c6ccccc6)cc5)c5cccc6c5oc5ccccc56)c4)cc1c32 ; CC1(C)c2ccccc2N2c3ccccc3Oc3c(-c4ccc(N(c5ccccc5)c5ccccc5)cc4)ccc1c32 ; CC1(C)c2ccccc2N2c3ccccc3Oc3c(-c4ccc(N(c5ccccc5)c5ccc(-c6ccccc6)cc5)cc4)ccc1c32 ; CC1(C)c2ccccc2N2c3ccccc3Oc3c(-c4ccc(N(c5ccc(-c6ccccc6)cc5)c5ccc(-c6ccccc6)cc5)cc4)ccc1c32]

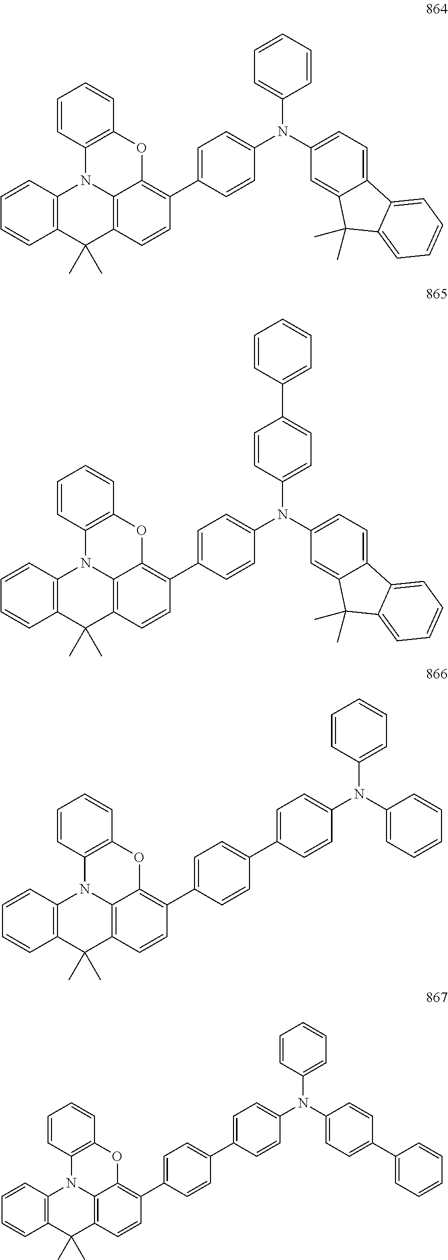 [SMILES: CC1(C)c2ccccc2-c2ccc(N(c3ccccc3)c3ccc(-c4ccc5c6c4Oc4ccccc4N6c4ccccc4C5(C)C)cc3)cc21 ; CC1(C)c2ccccc2-c2ccc(N(c3ccc(-c4ccccc4)cc3)c3ccc(-c4ccc5c6c4Oc4ccccc4N6c4ccccc4C5(C)C)cc3)cc21 ; CC1(C)c2ccccc2N2c3ccccc3Oc3c(-c4ccc(-c5ccc(N(c6ccccc6)c6ccccc6)cc5)cc4)ccc1c32 ; CC1(C)c2ccccc2N2c3ccccc3Oc3c(-c4ccc(-c5ccc(N(c6ccccc6)c6ccc(-c7ccccc7)cc6)cc5)cc4)ccc1c32]

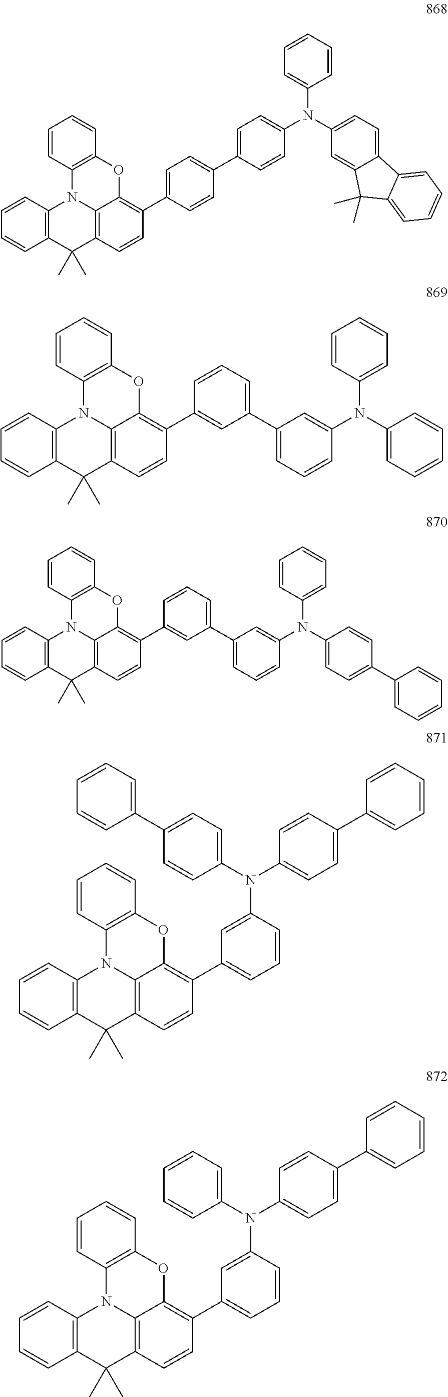

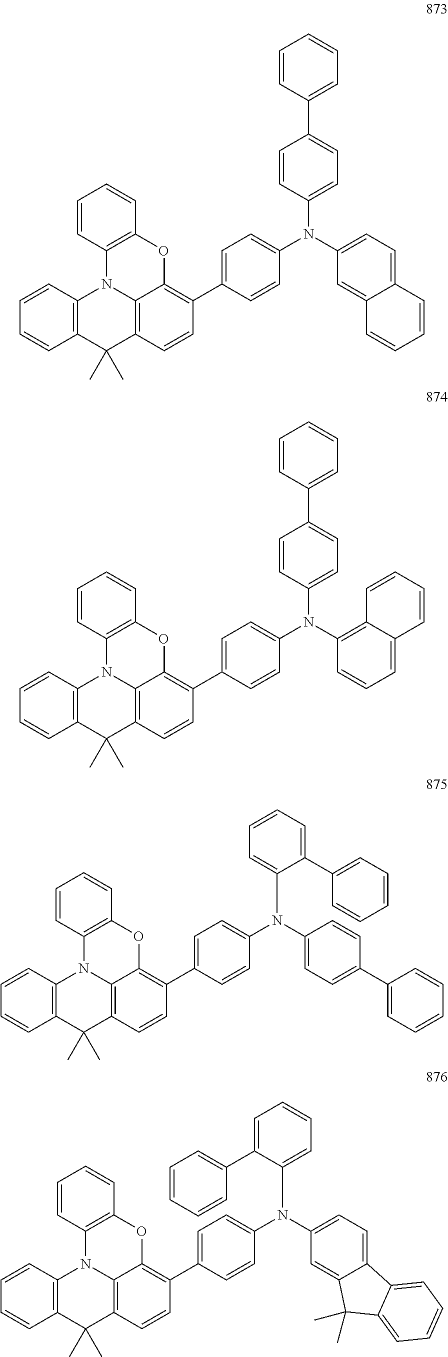 [SMILES: CC1(C)c2ccccc2N2c3ccccc3Oc3c(-c4ccc(N(c5ccc(-c6ccccc6)cc5)c5ccc6ccccc6c5)cc4)ccc1c32 ; CC1(C)c2ccccc2N2c3ccccc3Oc3c(-c4ccc(N(c5ccc(-c6ccccc6)cc5)c5cccc6ccccc56)cc4)ccc1c32 ; CC1(C)c2ccccc2N2c3ccccc3Oc3c(-c4ccc(N(c5ccc(-c6ccccc6)cc5)c5ccccc5-c5ccccc5)cc4)ccc1c32 ; CC1(C)c2ccccc2-c2ccc(N(c3ccc(-c4ccc5c6c4Oc4ccccc4N6c4ccccc4C5(C)C)cc3)c3ccccc3-c3ccccc3)cc21]

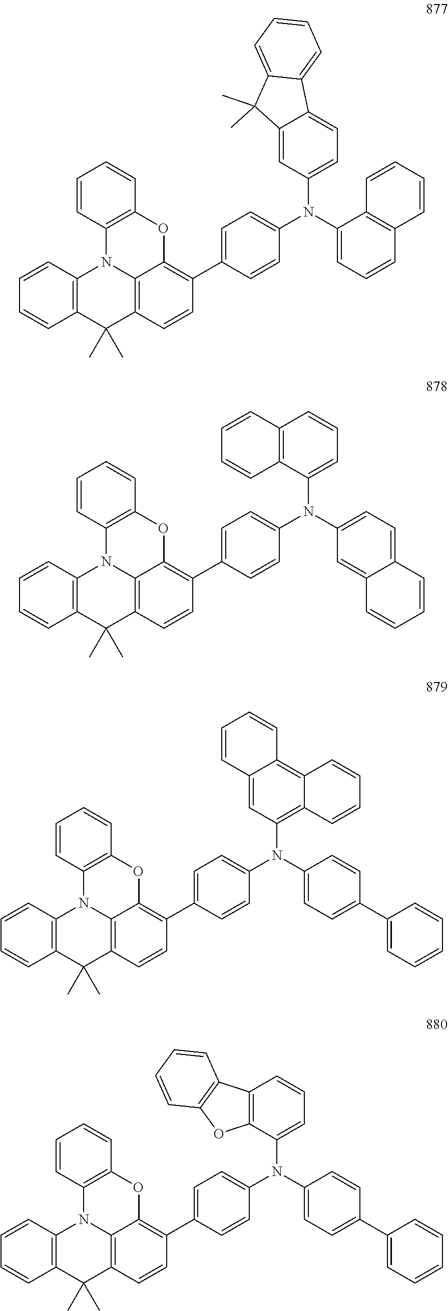 [SMILES: CC1(C)c2ccccc2-c2ccc(N(c3ccc(-c4ccc5c6c4Oc4ccccc4N6c4ccccc4C5(C)C)cc3)c3cccc4ccccc34)cc21 ; CC1(C)c2ccccc2N2c3ccccc3Oc3c(-c4ccc(N(c5ccc6ccccc6c5)c5cccc6ccccc56)cc4)ccc1c32 ; CC1(C)c2ccccc2N2c3ccccc3Oc3c(-c4ccc(N(c5ccc(-c6ccccc6)cc5)c5cc6ccccc6c6ccccc56)cc4)ccc1c32 ; CC1(C)c2ccccc2N2c3ccccc3Oc3c(-c4ccc(N(c5ccc(-c6ccccc6)cc5)c5cccc6c5oc5ccccc56)cc4)ccc1c32]

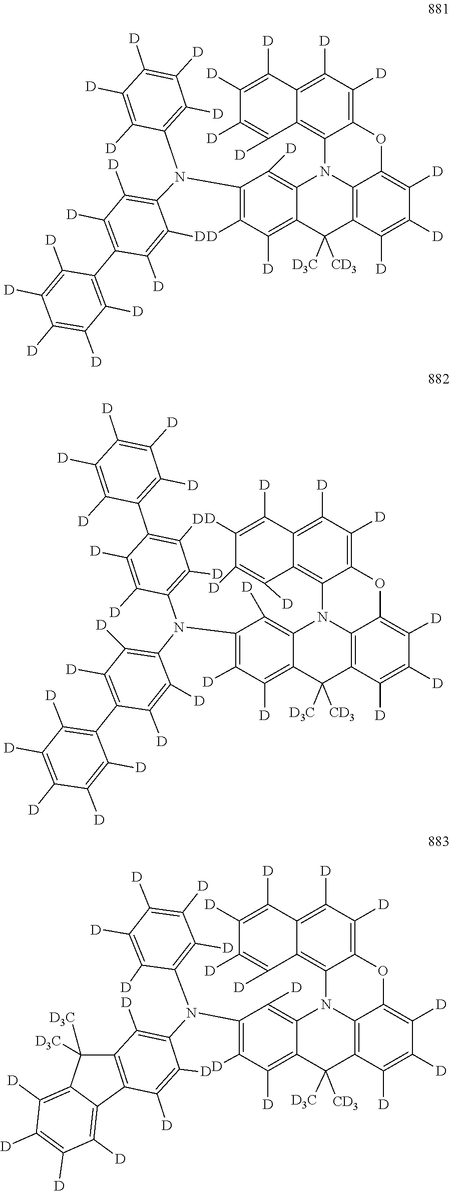

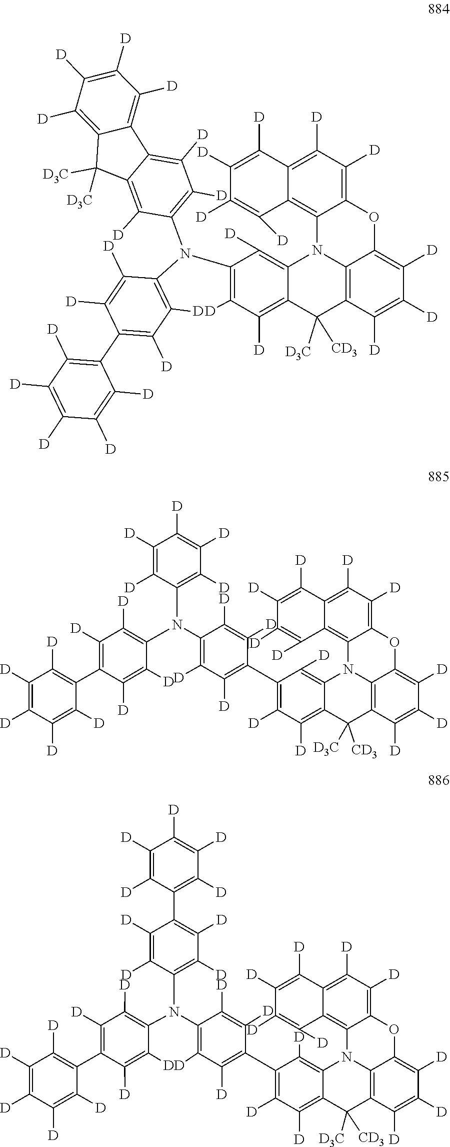

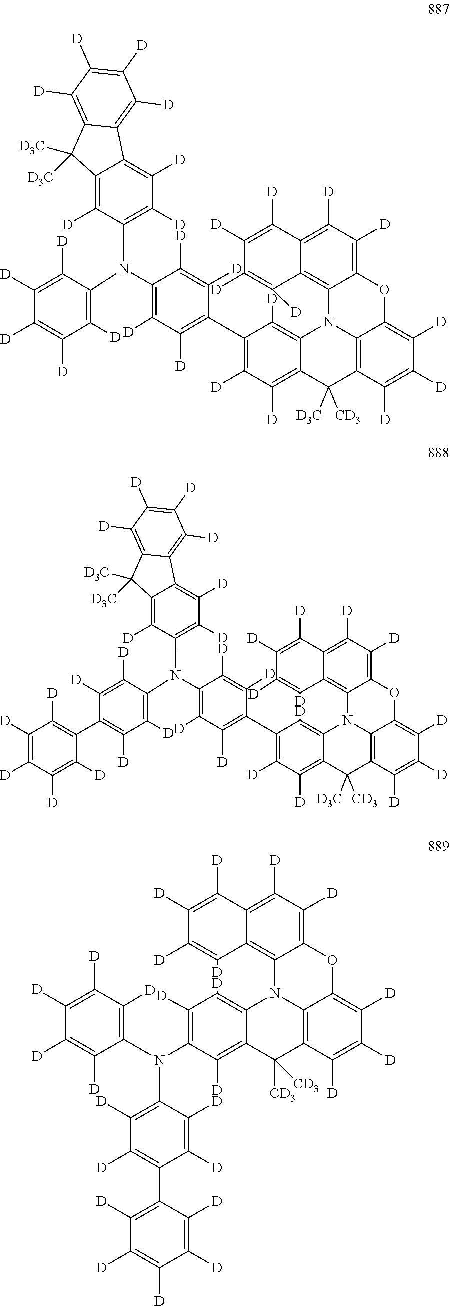

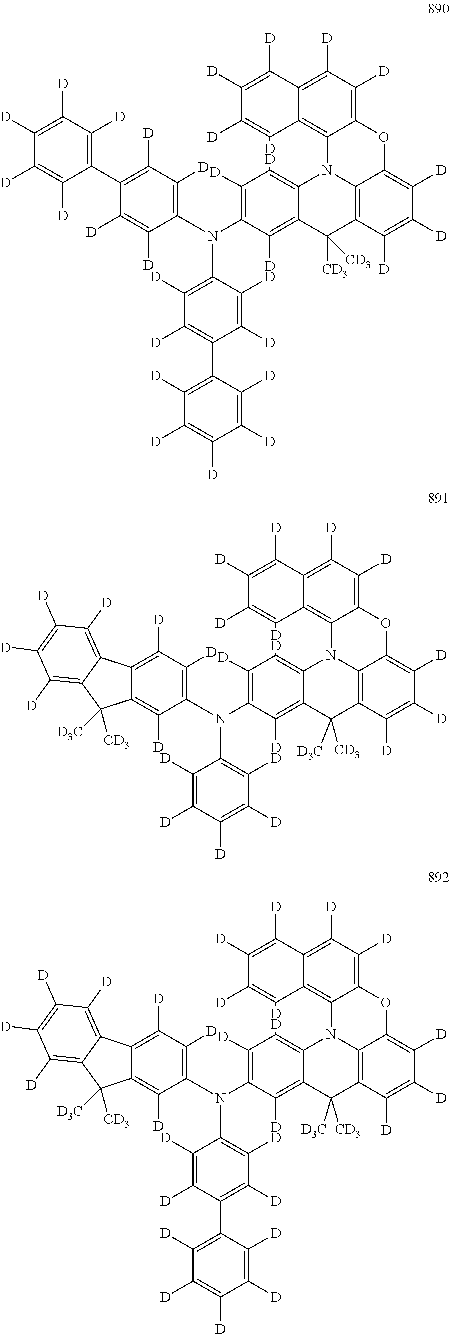

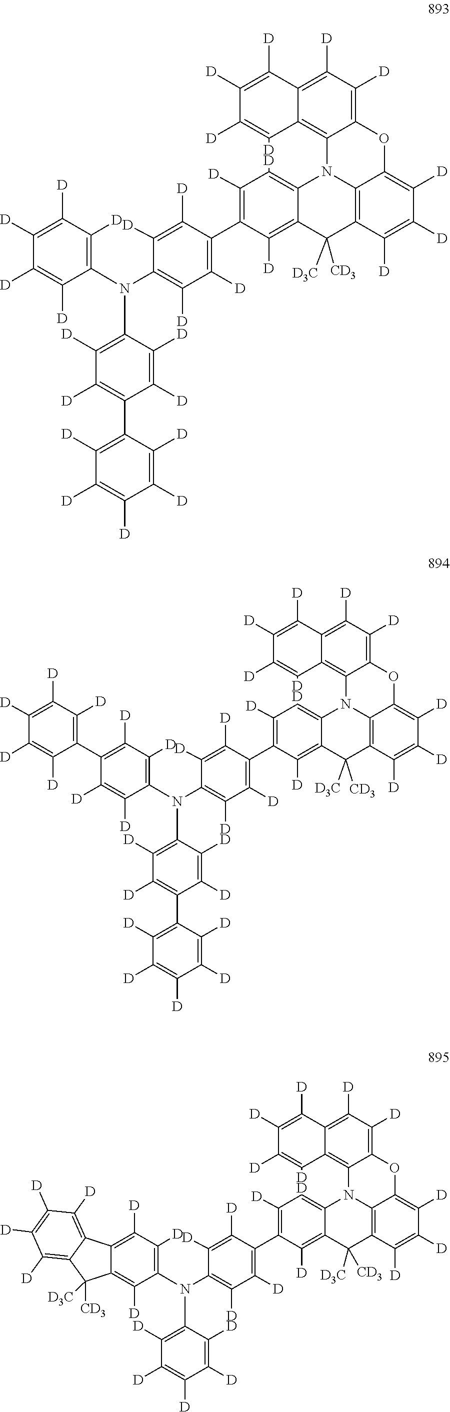

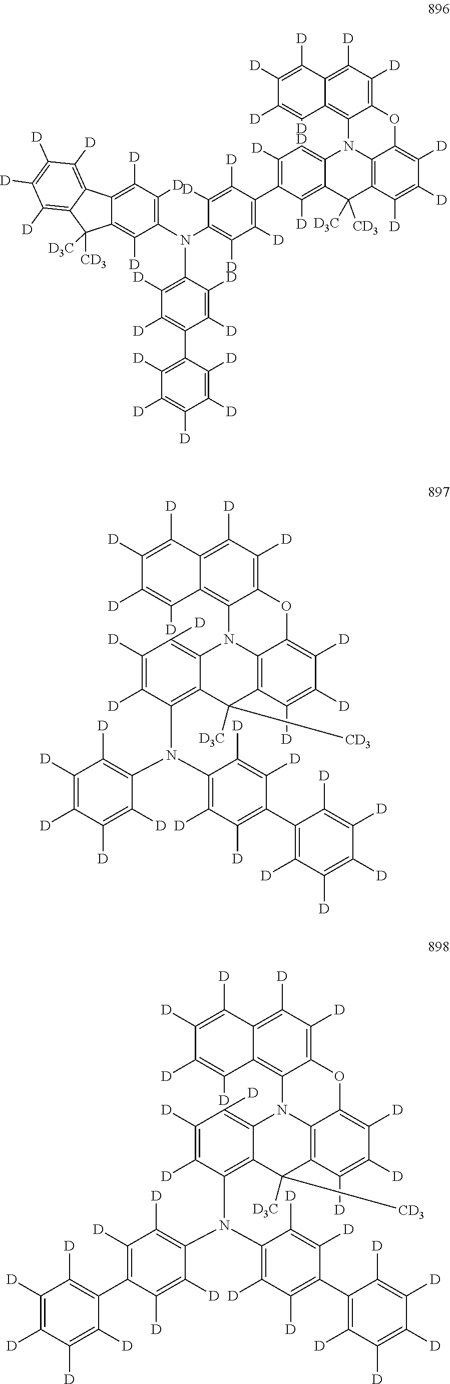

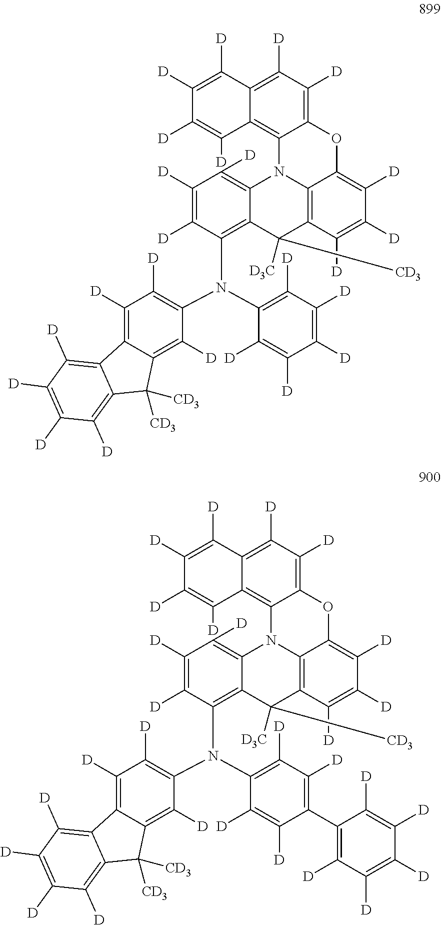

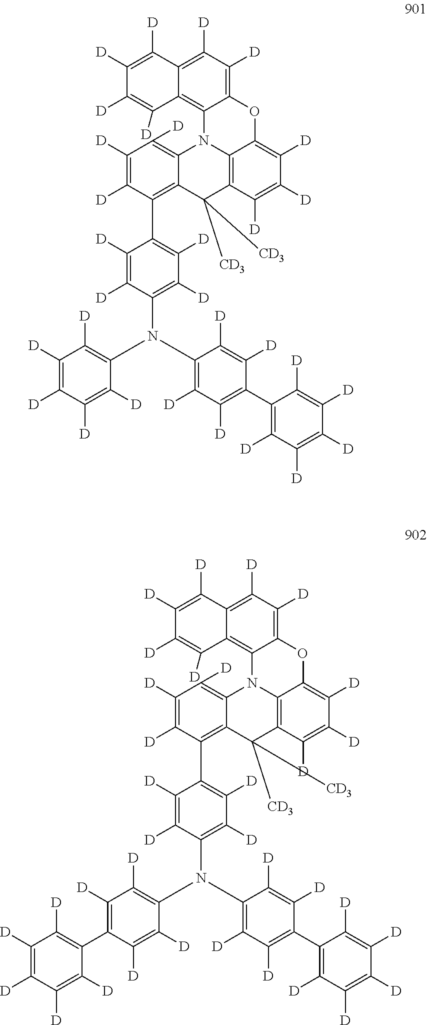

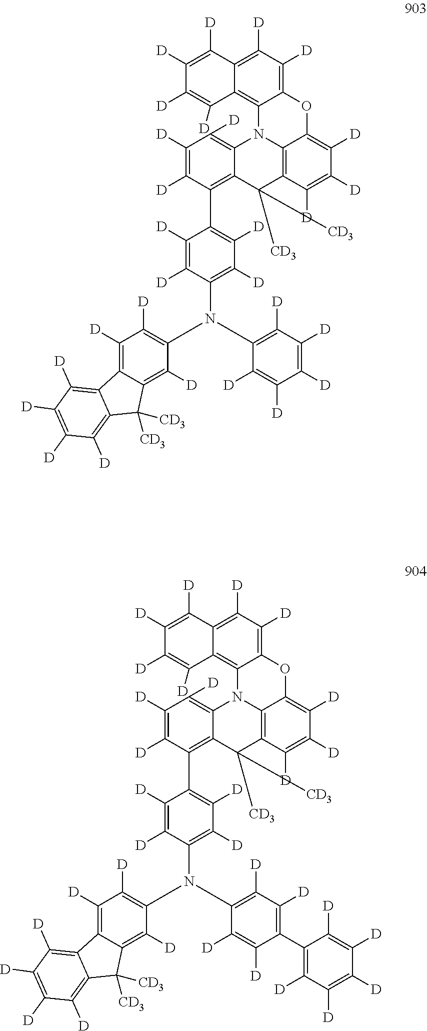

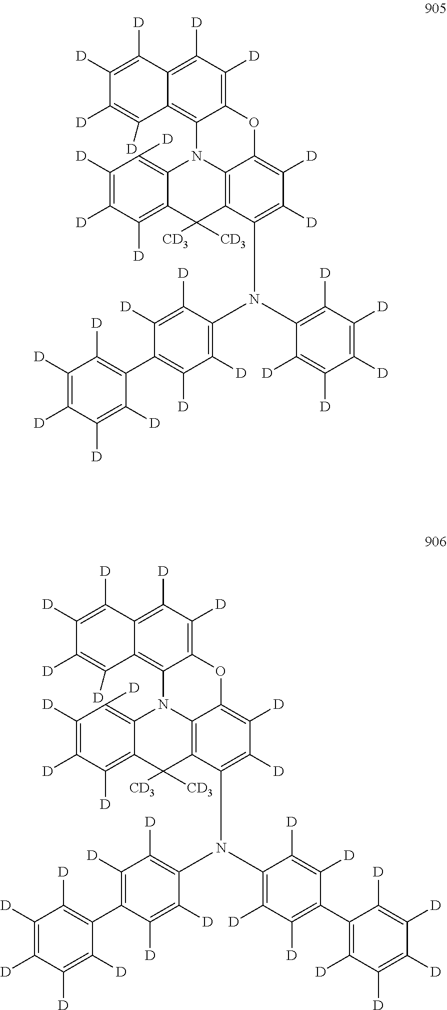

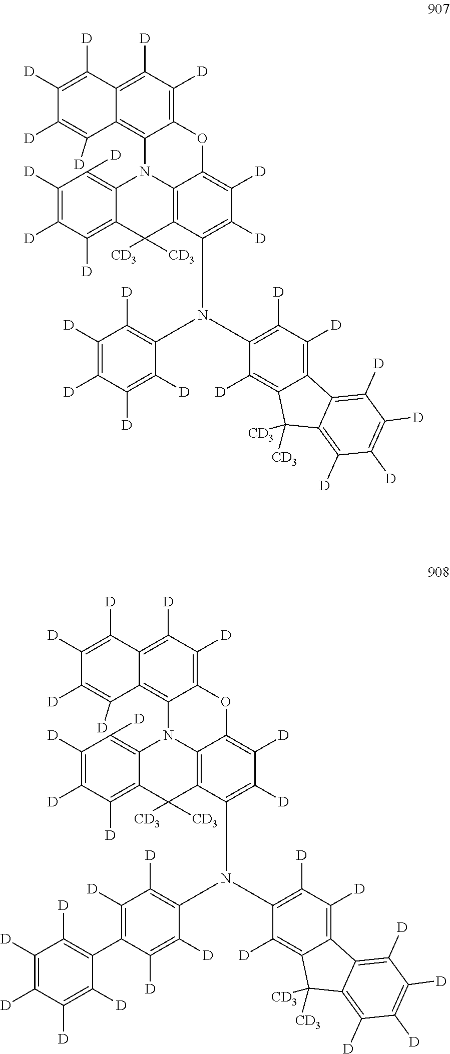

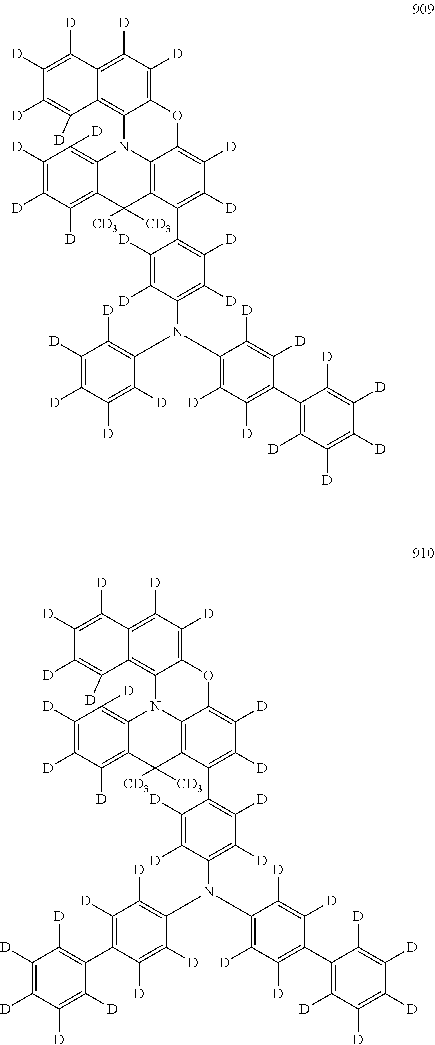

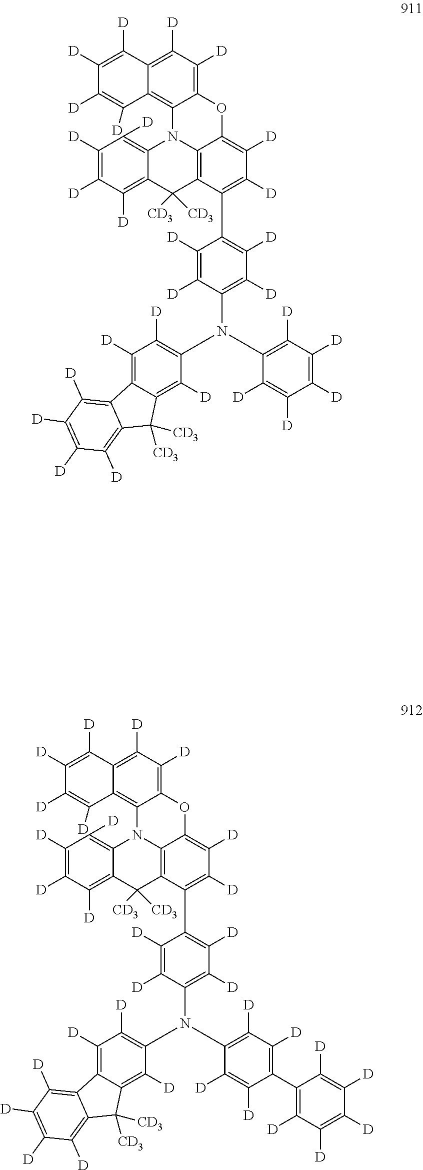

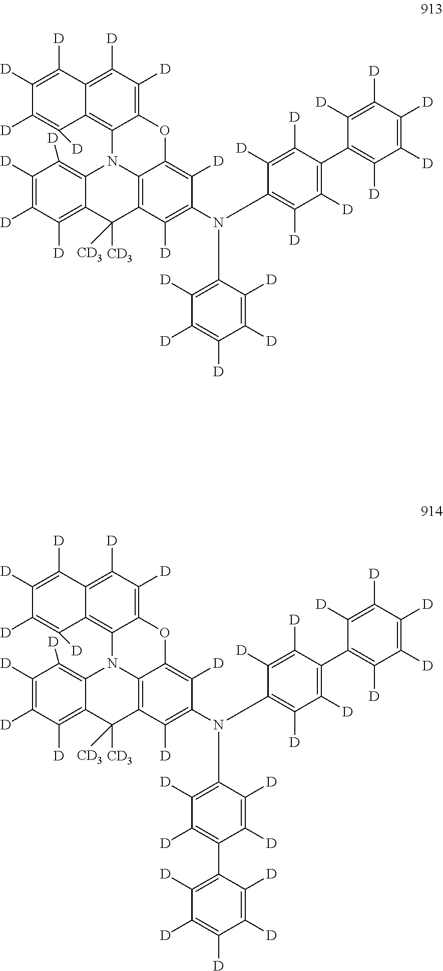

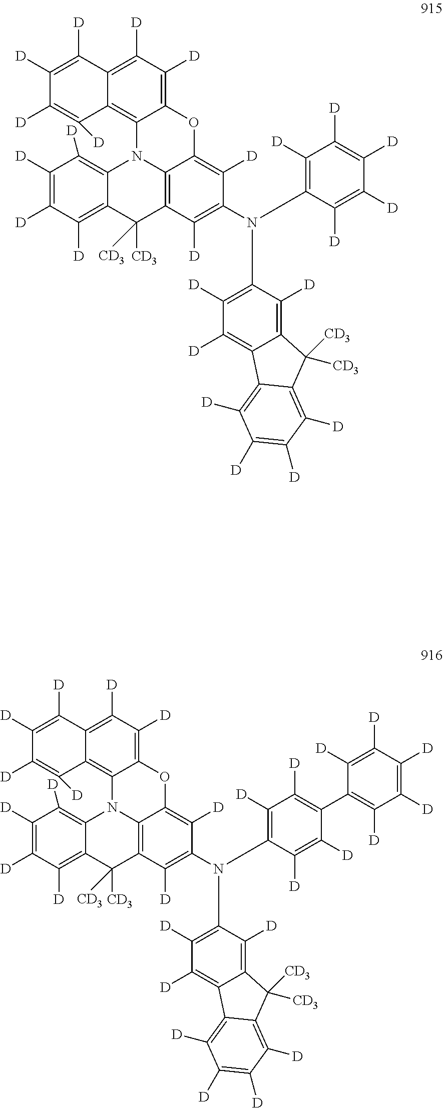

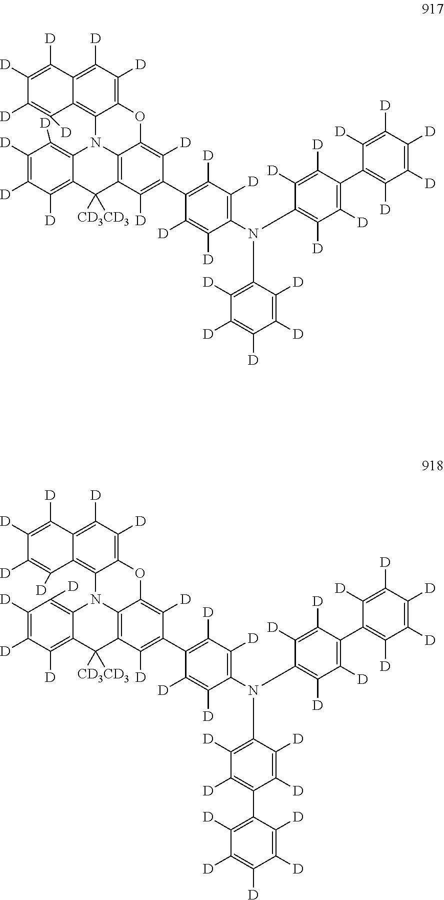

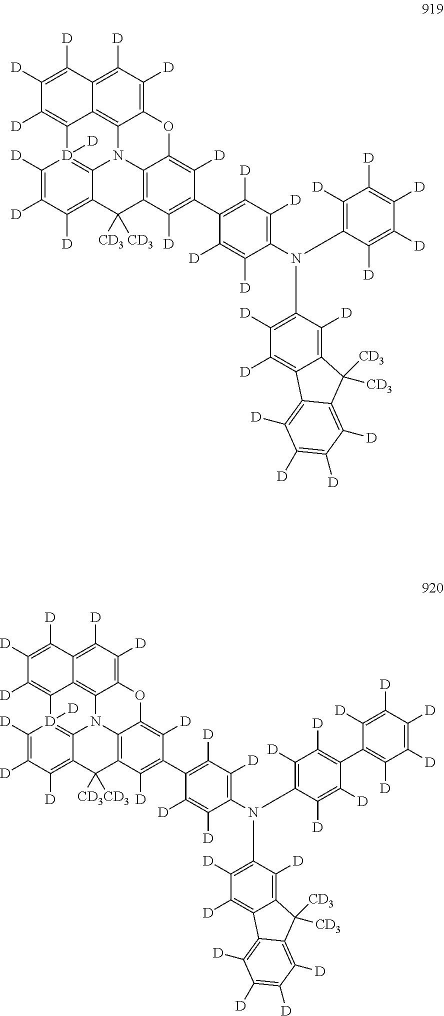

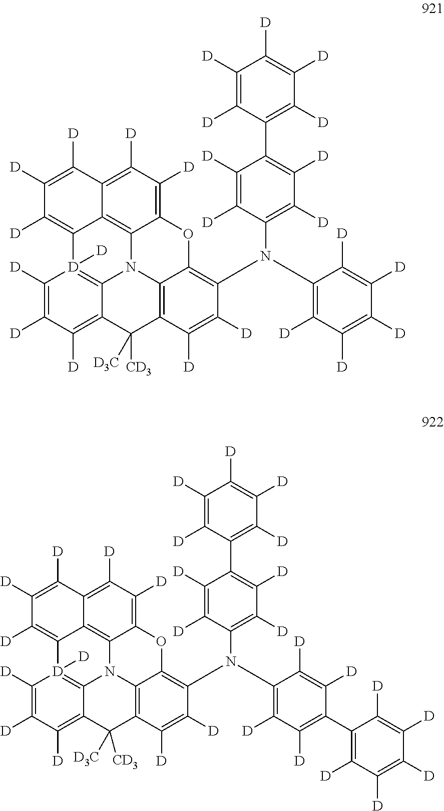

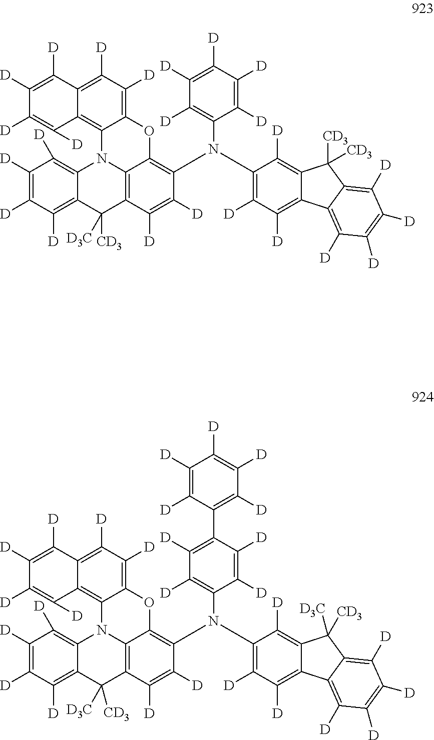

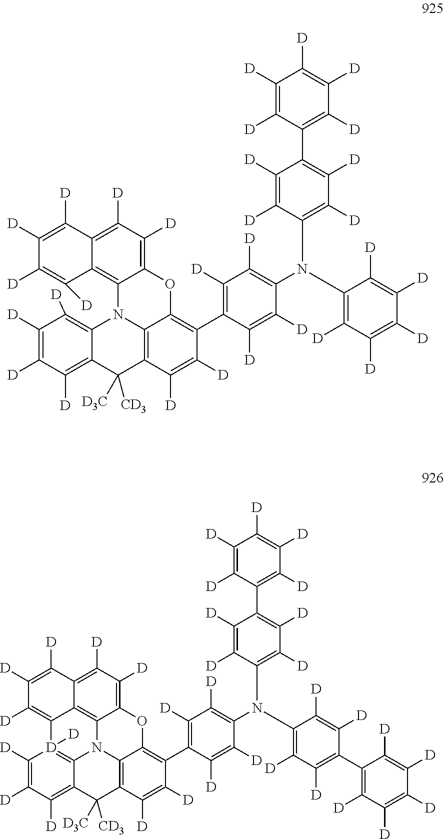

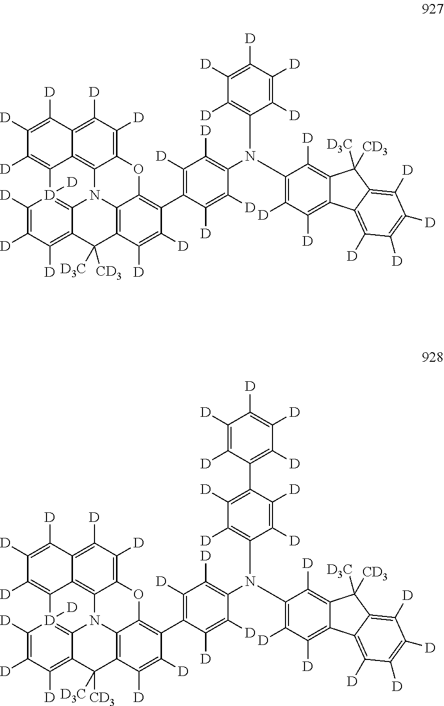

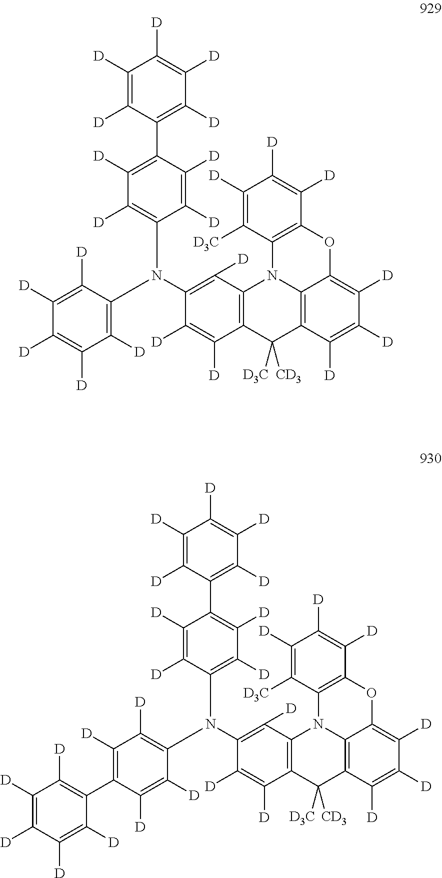

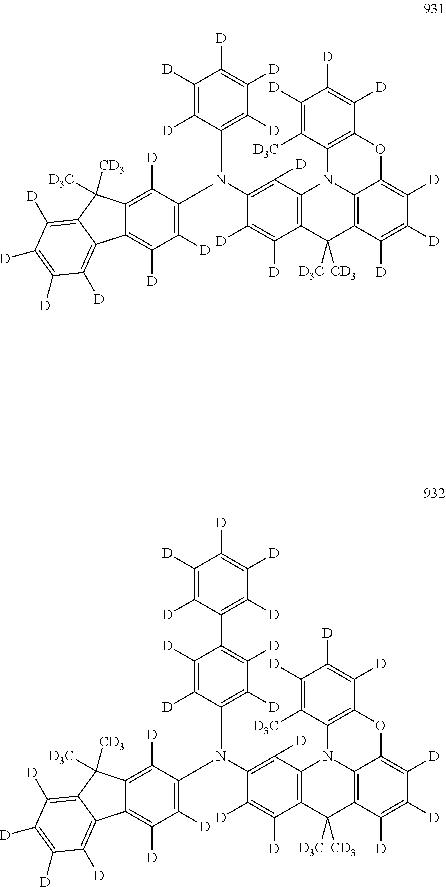

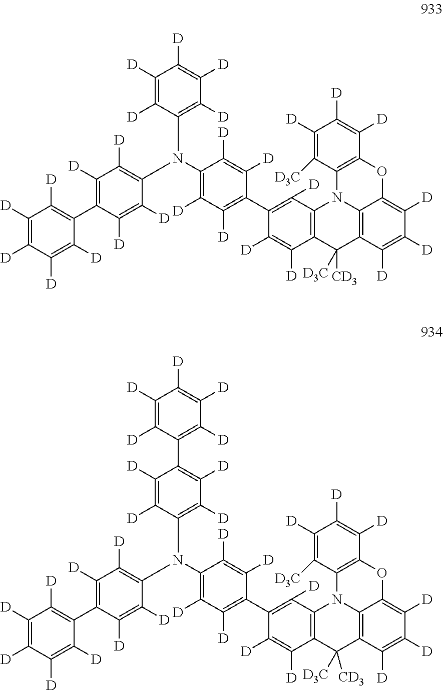

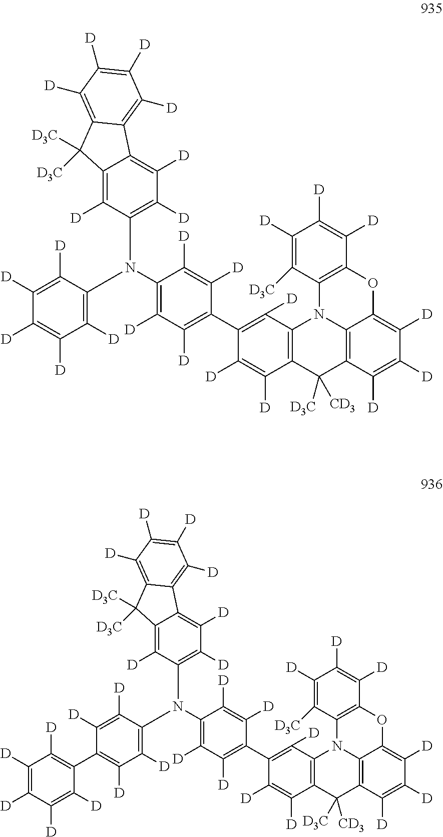

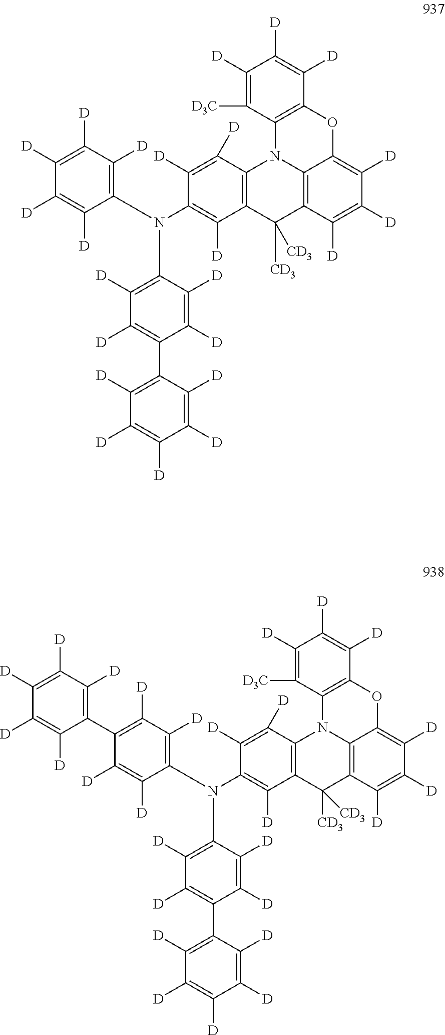

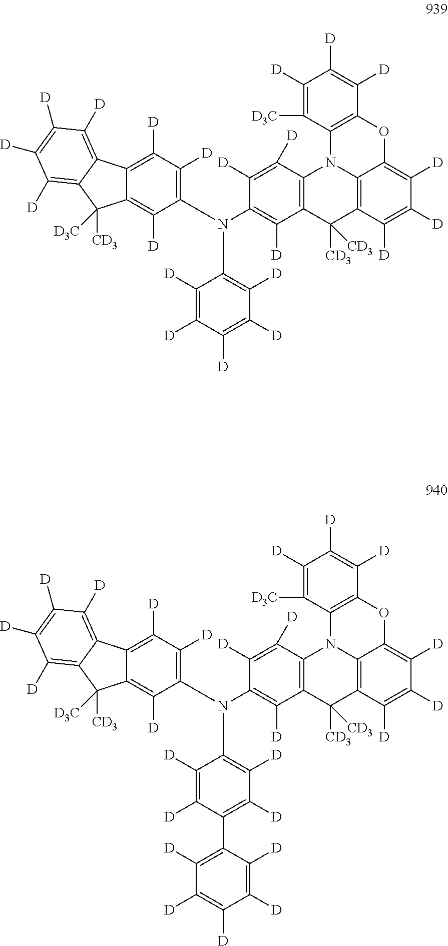

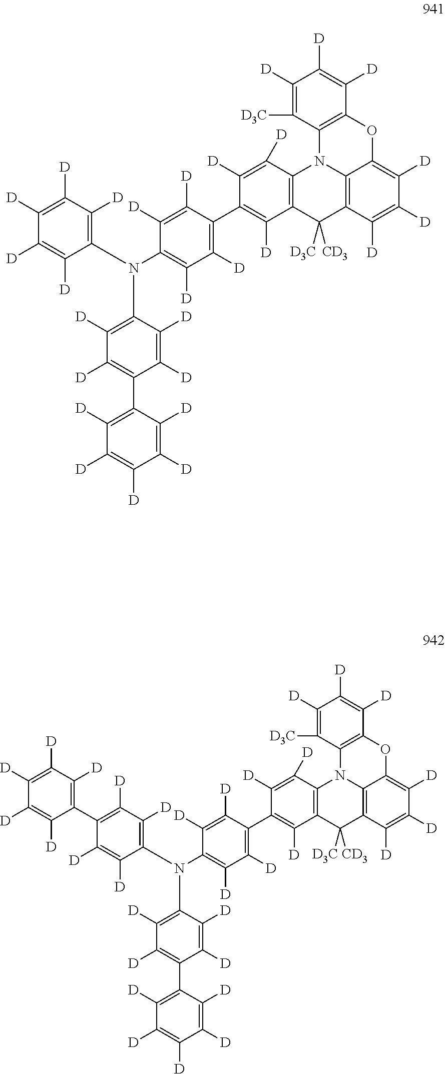

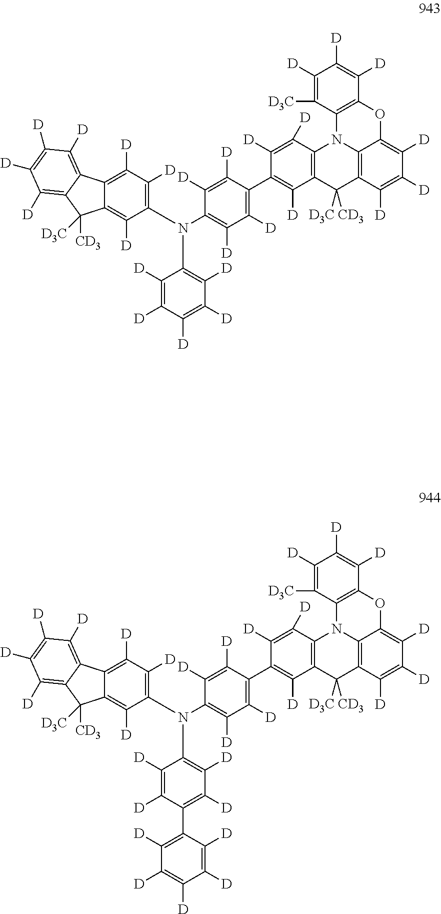

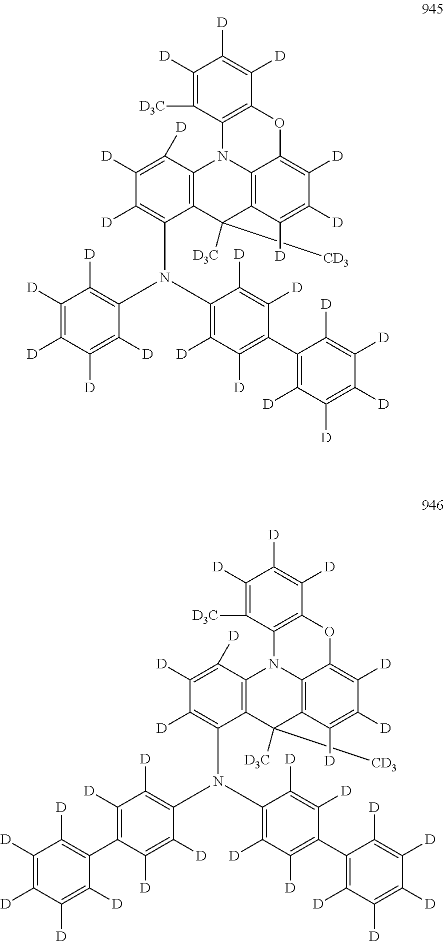

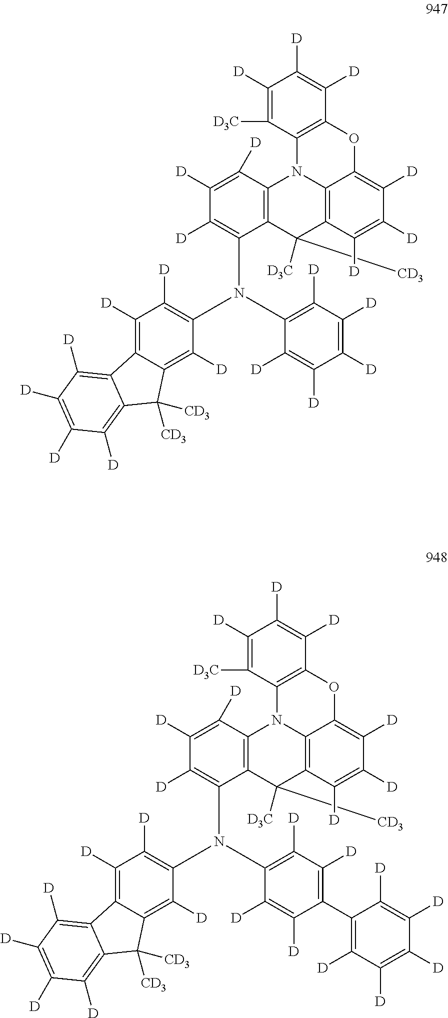

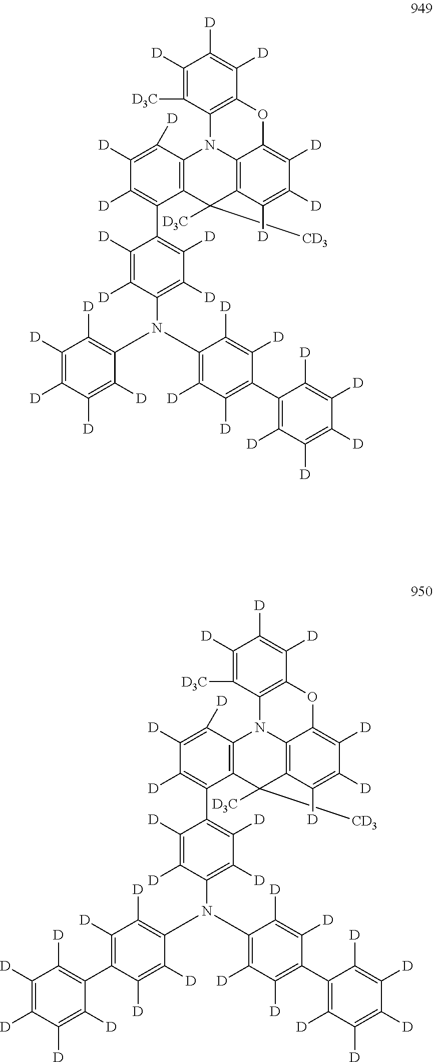

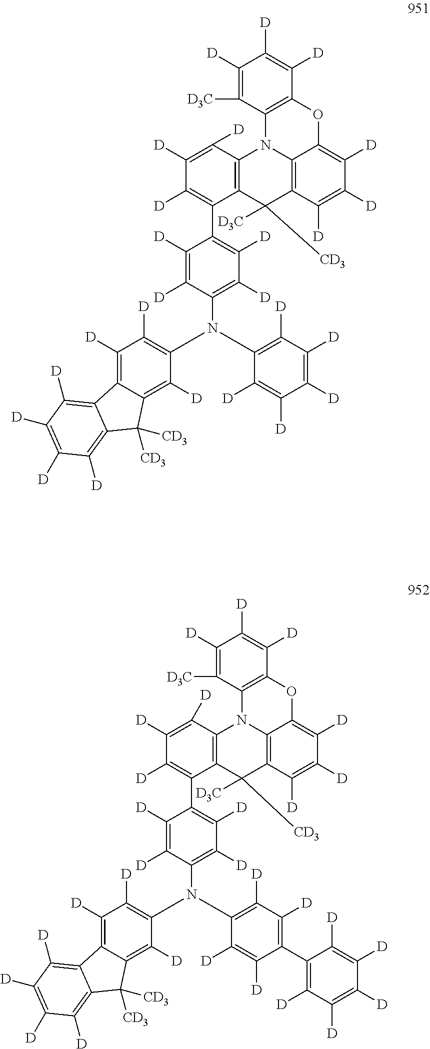

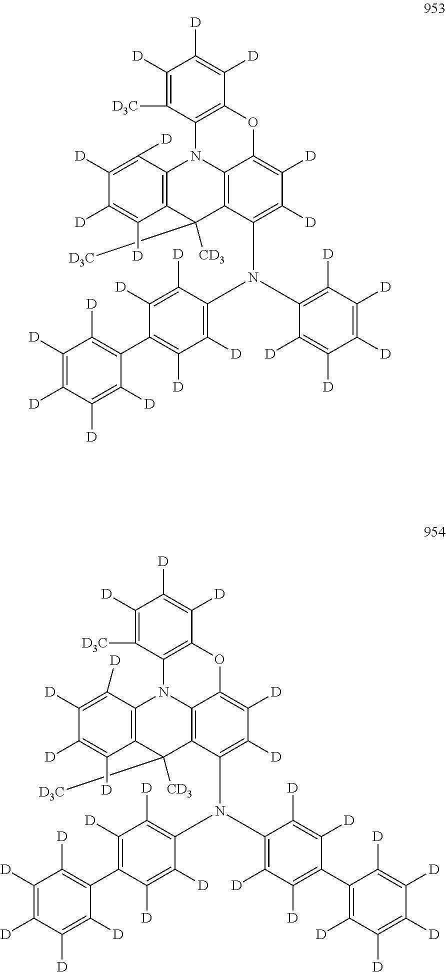

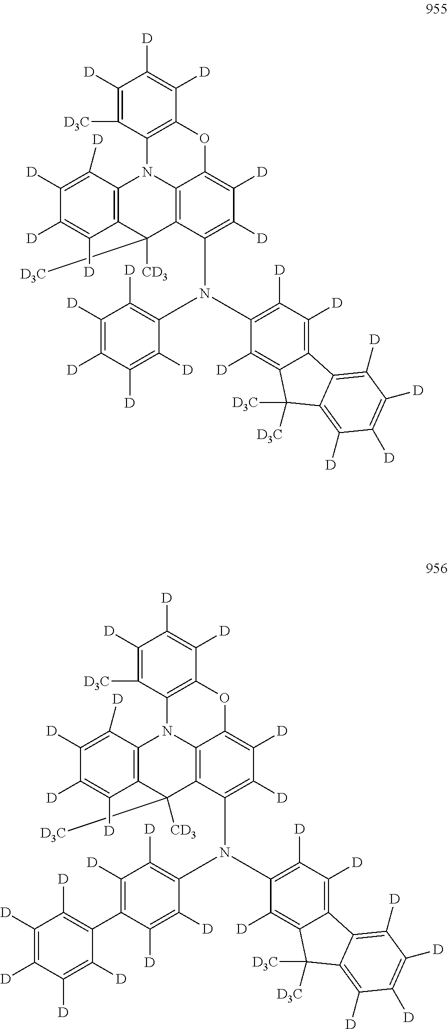

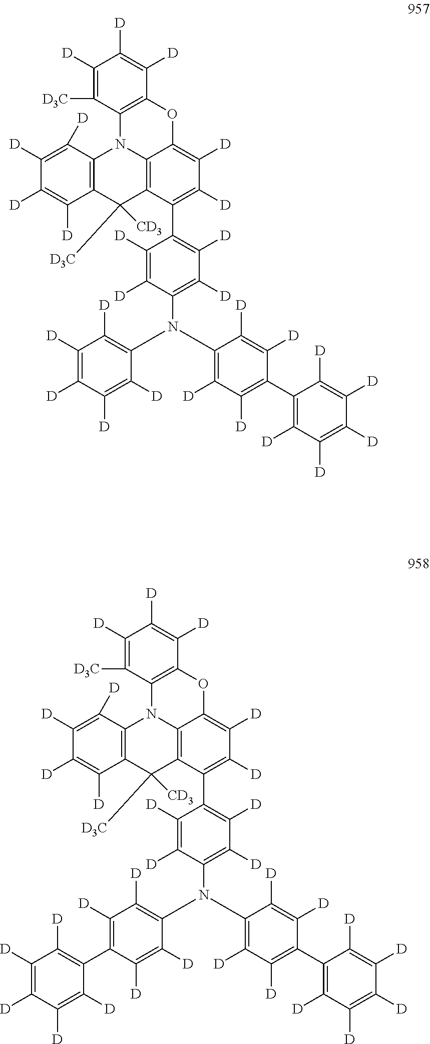

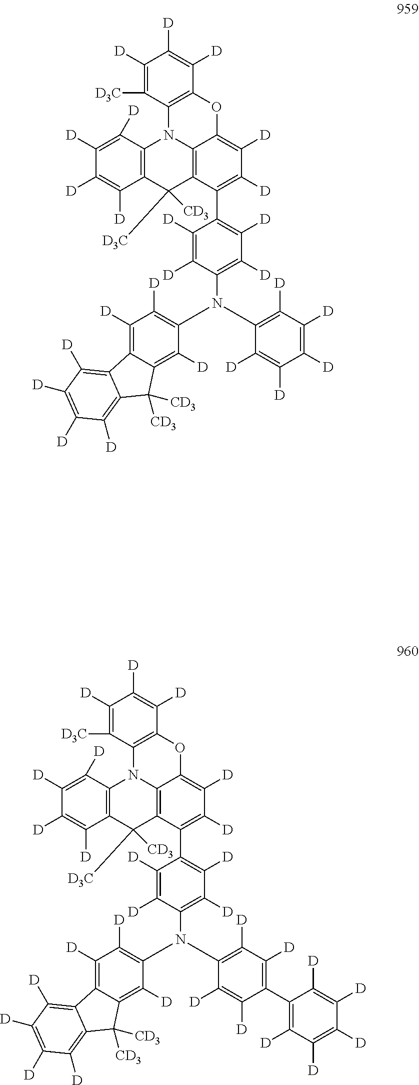

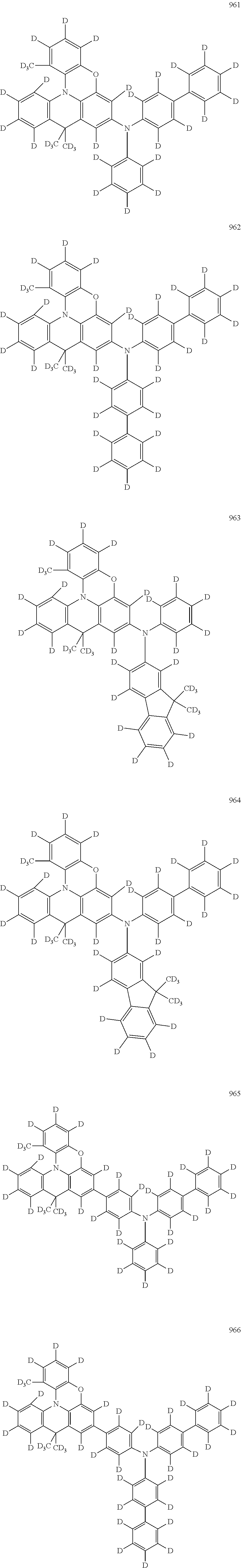

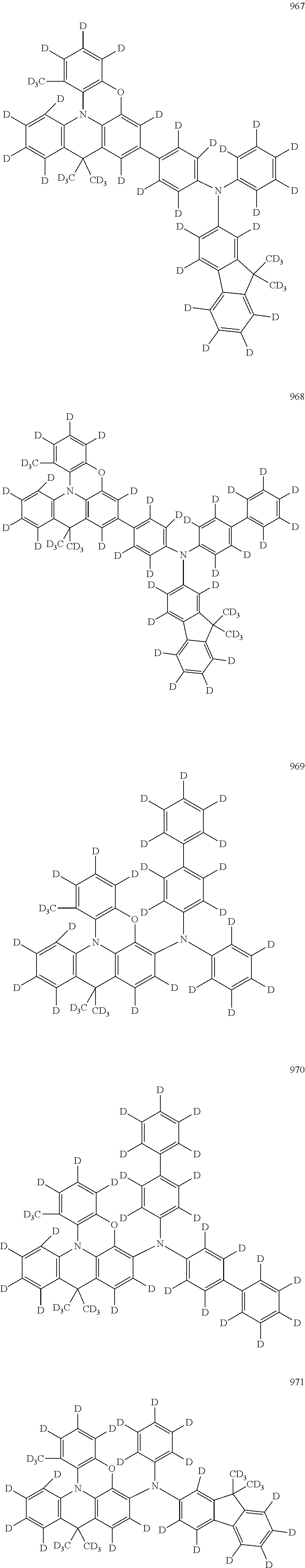

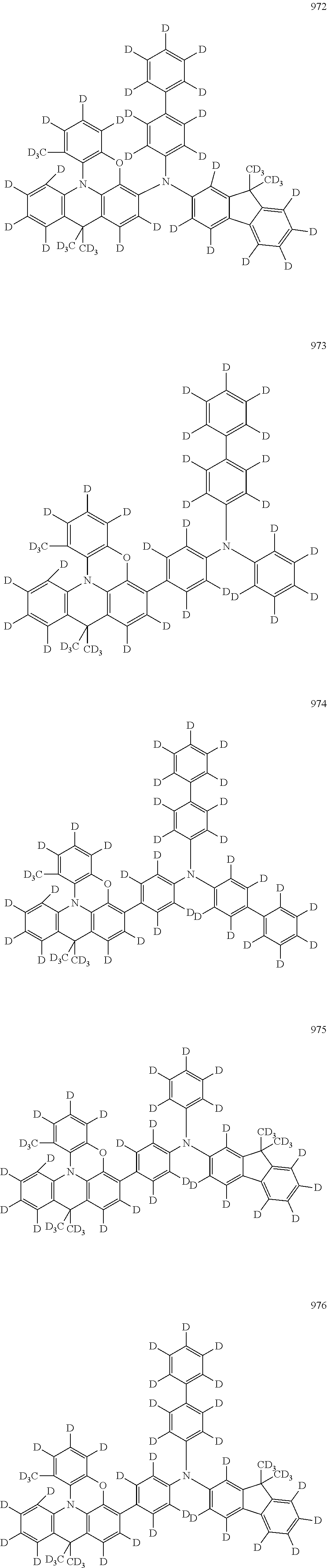

In addition, by introducing various substituents to the structure of Chemical Formula 1, compounds having unique properties of the introduced substituents may be synthesized. For example, by introducing substituents normally used as a hole injection layer material, an electron blocking layer material, a hole transfer layer material, a light emitting layer material, an electron transfer layer material, a hole blocking layer material and a charge generation layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

In addition, one embodiment of the present disclosure provides an organic light emitting device comprising:
a first electrode; a second electrode provided to face the first electrode; and one or more organic material layers provided between the first electrode and the second electrode, wherein one or more layers of the organic material layers include the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic light emitting device may be a blue organic light emitting device, and the heterocyclic compound represented by Chemical Formula 1 may be used as a material of the blue organic light emitting device.

In another embodiment of the present disclosure, the organic light emitting device may be a green organic light emitting device, and the heterocyclic compound represented by Chemical Formula 1 may be used as a material of the green organic light emitting device.

In another embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

In another embodiment of the present disclosure, the organic light emitting device may be a red organic light emitting device, and the heterocyclic compound represented by Chemical Formula 1 may be used as a light emitting layer material of the red organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transfer layer, a light emitting layer, an electron transfer layer, a hole blocking layer, an electron injection layer and the like as the organic material layer. However, the structure of the organic light emitting device is not limited thereto, and may include a smaller number of organic material layers.

In the organic light emitting device according to one embodiment of the present disclosure, the organic material layer including the heterocyclic compound represented by Chemical Formula 1 further includes a heterocyclic compound represented by the following Chemical Formula 3 or Chemical Formula 4.

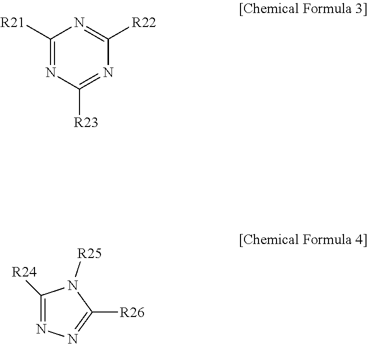

In Chemical Formula 3 and Chemical Formula 4,
R21 to R26 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group.

In one embodiment of the present disclosure, R21 to R26 are the same as or different from each other, and may be each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In another embodiment of the present disclosure, R21 to R26 are the same as or different from each other, and may be each independently hydrogen; deuterium; a substituted or unsubstituted C1 to C30 alkyl group; a substituted or unsubstituted C6 to C30 aryl group; or a substituted or unsubstituted C2 to C30 heteroaryl group.

In one embodiment of the present disclosure, R21 to R26 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In one embodiment of the present disclosure, R21 to R26 are the same as or different from each other, and may be each independently hydrogen; deuterium; a substituted or unsubstituted methyl group, ethyl group, propyl group, isopropyl group, butyl group or isobutyl group; a substituted or unsubstituted phenyl group, biphenyl group, naphthyl group, phenanthrenyl group or isochrysenyl group.

When including the compound represented by Chemical Formula 1 and the compound represented by any one of Chemical Formula 3 and Chemical Formula 4 at the same time, effects of more superior efficiency and lifetime are obtained. This may lead to a forecast that an exciplex phenomenon occurs when including the two compounds at the same time.

The exciplex phenomenon is a phenomenon of releasing energy having sizes of a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and a driving voltage may be lowered, which resultantly helps with enhancement in the lifetime. In other words, when using the compound represented by Chemical Formula 1 as the donor and using the compound represented by Chemical Formula 3 or Chemical Formula 4 as the acceptor, excellent device properties are obtained.

In one embodiment of the present disclosure, the heterocyclic compound represented by Chemical Formula 3 or Chemical Formula 4 may be one or more types selected from among the following compounds.

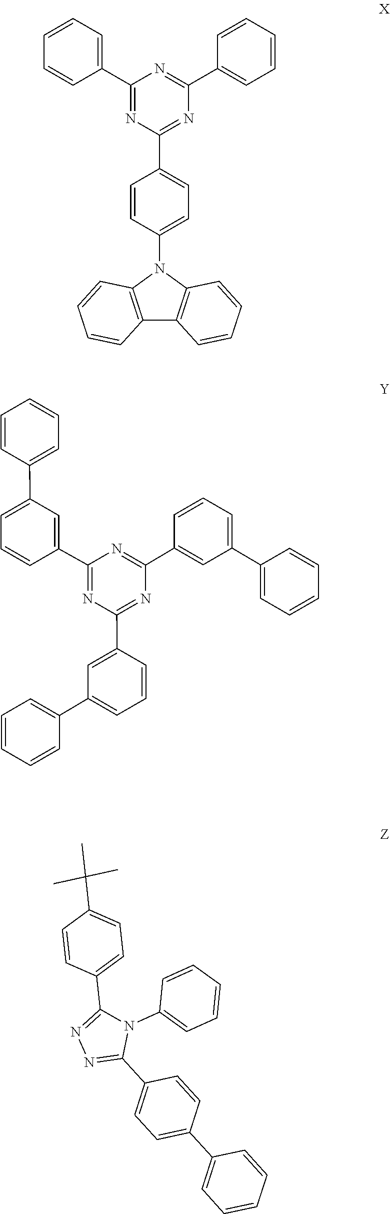 [SMILES: c1ccc(-c2nc(-c3ccccc3)nc(-c3ccc(-n4c5ccccc5c5ccccc54)cc3)n2)cc1 ; c1ccc(-c2cccc(-c3nc(-c4cccc(-c5ccccc5)c4)nc(-c4cccc(-c5ccccc5)c4)n3)c2)cc1 ; CC(C)(C)c1ccc(-c2nnc(-c3ccc(-c4ccccc4)cc3)n2-c2ccccc2)cc1]

In addition, one embodiment of the present disclosure provides a composition for an organic material layer of an organic light emitting device, the composition including the heterocyclic compound represented by Chemical Formula 1 and the heterocyclic compound represented by Chemical Formula 3 or Chemical Formula 4.

Specific descriptions on the heterocyclic compound represented by Chemical Formula 1 and the heterocyclic compound represented by Chemical Formula 3 or Chemical Formula 4 are the same as the descriptions provided above.

In one embodiment of the present disclosure, the heterocyclic compound represented by Chemical Formula 1 and the heterocyclic compound represented by Chemical Formula 3 or Chemical Formula 4 may have a weight ratio of 1:10 to 10:1, 1:8 to 8:1, 1:5 to 5:1 or 1:2 to 2:1 in the composition for an organic material layer of an organic light emitting device, however, the ratio is not limited thereto.

The composition for an organic material layer of an organic light emitting device may be used when forming an organic material of an organic light emitting device, and particularly, may be more preferably used when forming a host of a hole transfer layer, an electron blocking layer or a light emitting layer.

In one embodiment of the present disclosure, the organic material layer includes the heterocyclic compound represented by Chemical Formula 1 and the heterocyclic compound represented by Chemical Formula 3 or Chemical Formula 4, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited to these examples.

M may be iridium, platinum, osmium or the like.

L is an anionic bidentate ligand coordinated to M by sp2 carbon and heteroatom, and X may function to trap electrons or holes. Nonlimiting examples of L may include 2-(1-naphthyl)benzoxazole, 2-phenylbenzoxazole, 2-phenylbenzothiazole, 7,8-benzoquinoline, phenylpyridine, benzothiophene group pyridine, 3-methoxy-2-phenylpyridine, thiophene group pyridine, tolylpyridine and the like.

Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are described below, however, the phosphorescent dopant is not limited to these examples.

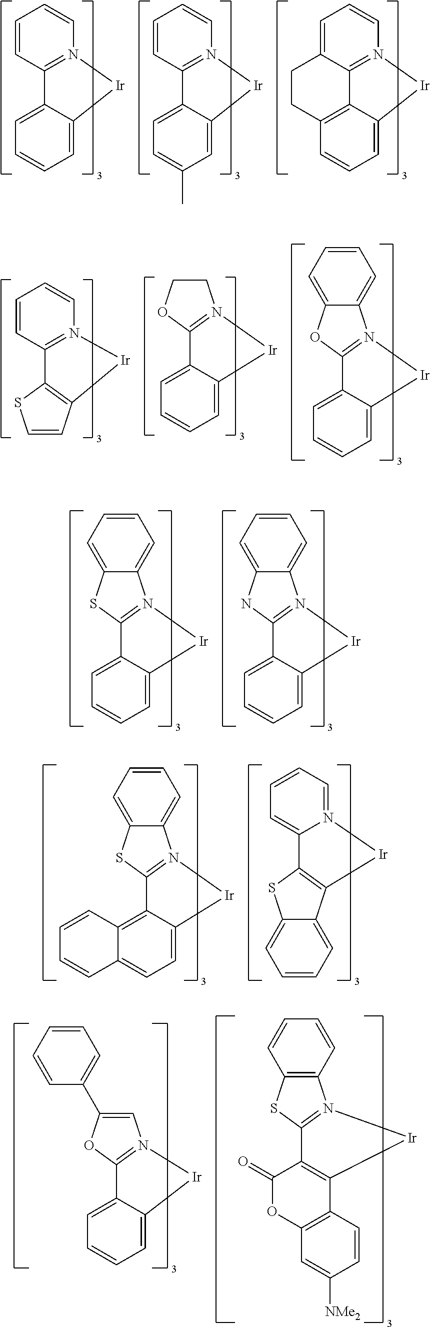

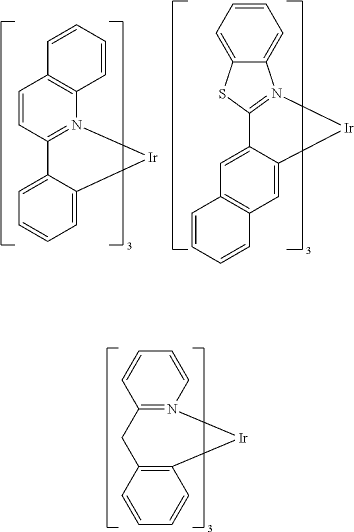

In one embodiment of the present disclosure, the organic material layer includes the heterocyclic compound represented by Chemical Formula 1 and the heterocyclic compound represented by Chemical Formula 3 or Chemical Formula 4, and an iridium-based dopant may be used therewith.

In one embodiment of the present disclosure, as the iridium-based dopant, Ir(ppy)₃ may be used as a green phosphorescent dopant and (piq)₂(Ir) (acac) may be used as a red phosphorescent dopant.

In one embodiment of the present disclosure, a content of the dopant may be from 1% to 15%, preferably from 3% to 10% and more preferably from 3% to 7% with respect to the weight of the host material.

In the organic light emitting device according to one embodiment of the present disclosure, the organic material layer includes an electron injection layer or an electron transfer layer, and the electron injection layer or the electron transfer layer may include the heterocyclic compound.

In the organic light emitting device according to another embodiment, the organic material layer includes an electron blocking layer or a hole blocking layer, and the electron blocking layer or the hole blocking layer may include the heterocyclic compound.

In the organic light emitting device according to another embodiment, the organic material layer includes an electron transfer layer, a light emitting layer or a hole blocking layer, and the electron transfer layer, the light emitting layer or the hole blocking layer may include the heterocyclic compound.

In the organic light emitting device according to another embodiment, the organic material layer includes a light emitting layer, and the light emitting layer may include the heterocyclic compound.

The organic light emitting device according to one embodiment of the present disclosure may further include one, two or more layers selected from the group consisting of a light emitting layer, a hole injection layer, a hole transfer layer, an electron injection layer, an electron transfer layer, an electron blocking layer and a hole blocking layer.

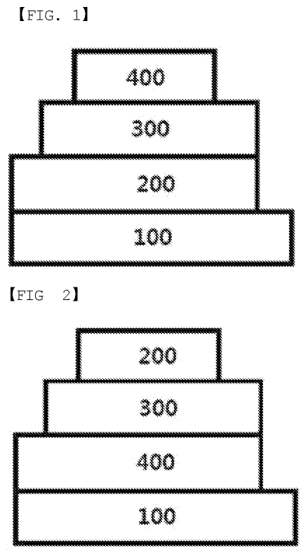

FIG. 1 to FIG. 3 illustrate a lamination order of electrodes and organic material layers of the organic light emitting device according to one embodiment of the present disclosure. However, the scope of the present application is not limited to these diagrams, and structures of organic light emitting devices known in the art may also be used in the present application.

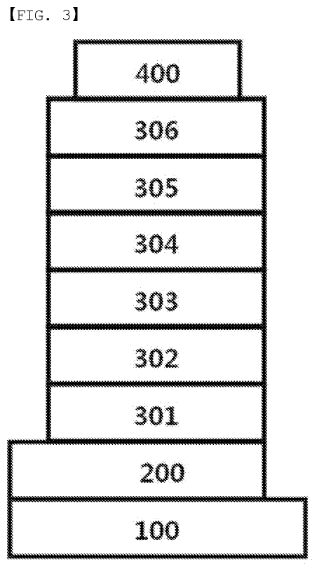

FIG. 1 illustrates the organic light emitting device in which a positive electrode (200), an organic material layer (300) and a negative electrode (400) are consecutively laminated on a substrate (100). However, the structure is not limited to such a structure, and as illustrated in FIG. 2, an organic light emitting device in which a negative electrode, an organic material layer and a positive electrode are consecutively laminated on a substrate may also be obtained.

FIG. 3 illustrates a case of the organic material layer being a multilayer. The organic light emitting device according to FIG. 3 includes a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, the layers other than the light emitting layer may not be included, and other necessary functional layers may be further added.

One embodiment of the present disclosure provides a method for manufacturing an organic light emitting device, the method comprising the steps of:
preparing a substrate; forming a first electrode on the substrate; forming one or more organic material layers on the first electrode; and forming a second electrode on the one or more organic material layers, wherein the forming of organic material layers includes forming the one or more organic material layers using the composition for an organic material layer according to one embodiment of the present disclosure.

In one embodiment of the present disclosure, the forming of organic material layers may be forming using a thermal vacuum deposition method after pre-mixing the heterocyclic compound represented by Chemical Formula 1 and the heterocyclic compound represented by Chemical Formula 3 or Chemical Formula 4.

The pre-mixing means first mixing the heterocyclic compound represented by Chemical Formula 1 and the heterocyclic compound represented by Chemical Formula 3 or Chemical Formula 4 in one source of supply before depositing on the organic material layer.

The pre-mixed material may be referred to as the composition for an organic material layer according to one embodiment of the present application.

The organic material layer including the heterocyclic compound represented by Chemical Formula 1 may further include other materials as necessary.

The organic material layer including the heterocyclic compound represented by Chemical Formula 1 and the heterocyclic compound represented by Chemical Formula 3 or Chemical Formula 4 at the same time may further include other materials as necessary.

In the organic light emitting device according to one embodiment of the present disclosure, materials other than the heterocyclic compound represented by Chemical Formula 1 or the heterocyclic compound represented by Chemical Formula 3 or Chemical Formula 4 are illustrated below, however, these are for illustrative purposes only and not for limiting the scope of the present application, and these materials may be replaced by materials known in the art.

As the positive electrode material, materials having relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO2:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials having relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p.677 (1994)], conductive polymers having solubility such as polyaniline/dodecylbenzene sulfonic acid or poly(3,4-ethylenedioxythiophene)/poly(4-styrene-sulfonate), polyaniline/camphor sulfonic acid or polyaniline/poly(4-styrenesulfonate), and the like, may be used.

As the hole transfer layer material, pyrazoline derivatives, arylamine-based derivatives, stilbene derivatives, triphenyldiamine derivatives and the like may be used, and low molecular or high molecular materials may also be used.

As the electron transfer layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light by binding holes and electrons injected from a positive electrode and a negative electrode, respectively, may be used alone, however, materials having a host material and a dopant material involving together in light emission may also be used.

When mixing hosts of the light emitting layer material, same series hosts may be mixed, or different series hosts may be mixed. For example, any two or more types of materials among n-type host materials or p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a similar principle used in the organic light emitting device.

Hereinafter, preferred examples are provided to illuminate the present disclosure, however, the following examples are provided to more readily understand the present disclosure, and the present disclosure is not limited thereto.

### Preparation Example

### <Preparation Example 1> Preparation of Compound 002-P

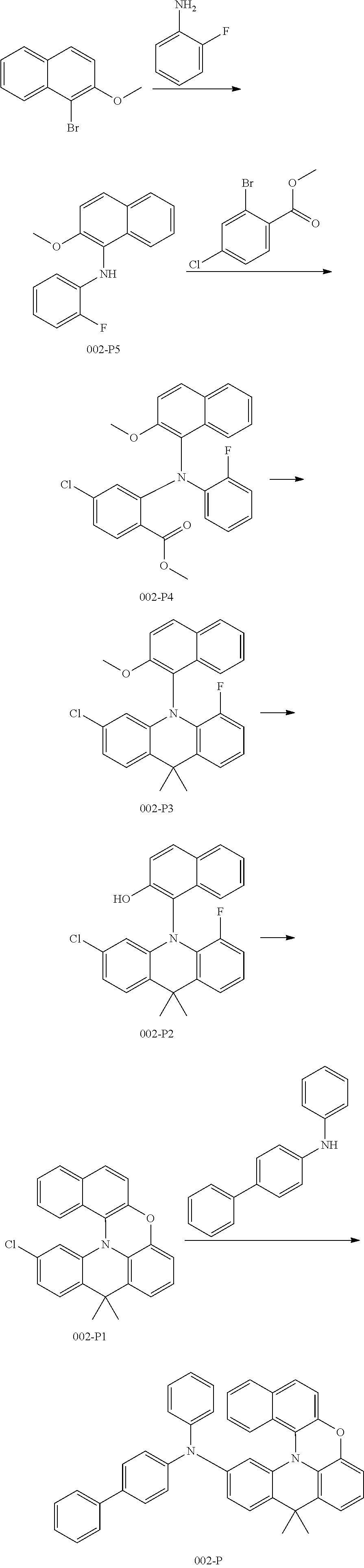

1) Preparation of Compound 002-P5

Dissolving 1-bromo-2-methoxynaphthalene (50 g, 210.89 mmol) and 2-fluoroaniline (30.46 g, 274.16 mmol) in toluene (500 ml), palladium(II) acetate (Pd(OAc)₂) (0.95 g, 4.22 mmol), 4,5-bis(diphenylphosphino)-9,9-dimethylxanthene (xantphos) (6.1 g, 10.54 mmol) and t-BuONa (40.53 g, 421.78 mmol) were introduced thereto, and the mixture was stirred under reflux for 2 hours. After the reaction was completed, dichloromethane was introduced to the reaction solution for dissolution. The result was extracted with distilled water, and after drying the organic layer with anhydrous MgSO₄, the solvent was removed using a rotary evaporator. The result was purified by column chromatography using dichloromethane and hexane as a developing solvent to obtain Compound 002-P5 (44 g, yield 78%).

2) Preparation of Compound 002-P4

Dissolving Compound 002-P5 (44 g, 164.61 mmol) and methyl 2-bromo-4-chlorobenzoate (53.39 g, 213.99 mmol) in toluene (500 ml), Pd(OAc)₂ (0.74 g, 3.29 mmol), xantphos (4.76 g, 8.23 mmol) and t-BuONa (31.64 g, 329.22 mmol) were introduced thereto, and the mixture was stirred under reflux for 2 hours. After the reaction was completed, dichloromethane was introduced to the reaction solution for dissolution. The result was extracted with distilled water, and after drying the organic layer with anhydrous MgSO₄, the solvent was removed using a rotary evaporator. The result was purified by column chromatography using dichloromethane and hexane as a developing solvent to obtain Compound 002-P4 (52 g, yield 72%).

3) Preparation of Compound 002-P3

Dissolving Compound 002-P4 (52 g, 119.30 mmol) in tetrahydrofuran (500 ml), methylmagnesium bromide (3 M solution in ether) (119 ml, 357.90 mmol) was slowly added thereto at 0° C., and the mixture was stirred for 6 hours at 60° C. After the reaction was finished, water was added to the reaction solution to terminate the reaction. The result was extracted using dichloromethane and distilled water, and after drying the organic layer with anhydrous MgSO₄, the solvent was removed using a rotary evaporator. After that, the result was dissolved in dichloromethane, boron trifluoride diethyl etherate was added to the reaction material, and the mixture was stirred for 4 hours at room temperature. After the reaction was finished, the result was purified by column chromatography using dichloromethane and hexane as a developing solvent to obtain Compound 002-P3 (40 g, yield 80%).

4) Preparation of Compound 002-P2

Dissolving Compound 002-P3 (40 g, 95.72 mmol) in dichloromethane (400 ml), boron tribromide (35.97 g, 143.58 mmol) was slowly added thereto at 0° C., and the mixture was stirred for 3 hours. After the reaction was finished, distilled water was slowly added to the reaction solution to terminate the reaction. The result was extracted with dichloromethane and distilled water, and after drying the organic layer with anhydrous MgSO₄, the solvent was removed using a rotary evaporator. The result was purified by column chromatography using dichloromethane and hexane as a developing solvent to obtain Compound 002-P2 (35 g, yield 91%).

5) Preparation of Compound 002-P1

Compound 002-P2 (35 g, 86.66 mmol) was dissolved in N,N-dimethylacetamide (400 ml), and after heating to 150° C., Cs₂CO₃ (56.47 g, 173.32 mmol) was introduced thereto, and the mixture was stirred under reflux for 30 minutes. After the reaction was completed, the result was extracted with dichloromethane and distilled water, and after drying the organic layer with anhydrous MgSO₄, the solvent was removed using a rotary evaporator. The result was purified by column chromatography using dichloromethane and hexane as a developing solvent to obtain Compound 002-P1 (24 g, yield 72%).

6) Preparation of Compound 002-P

Dissolving Compound 002-P1 (10 g, 26.05 mmol) and N-phenyl-[1,1′-biphenyl]-4-amine (6.39 g, 26.05 mmol) in toluene (100 ml), tris(dibenzylideneacetone)dipalladium (Pd₂(dba)₃) (0.48 g, 0.52 mmol), dicyclohexyl(2′,4′,6′-triisopropyl-[1,1′-biphenyl]-2-yl)phosphine (Xphos) (0.62 g, 1.30 mmol) and t-BuONa (5.01 g, 52.10 mmol) were introduced thereto, and the mixture was stirred under reflux for 2 hours. After the reaction was completed, dichloromethane was introduced to the reaction solution for dissolution. The result was extracted with distilled water, and after drying the organic layer with anhydrous MgSO₄, the solvent was removed using a rotary evaporator. The result was purified by column chromatography using dichloromethane and hexane as a developing solvent to obtain Compound 002 (11 g, yield 71%).

The following target compounds were synthesized in the same manner as in Preparation Example 1 except that Compound A was used instead of 1-bromo-2-methoxynaphthalene, Compound B was used instead of 2-fluoroaniline, Compound C was used instead of methyl 2-bromo-4-chlorobenzoate, and Compound D was used instead of N-phenyl-[1,1′-biphenyl]-4-amine.

TABLE 1

| | Compound | Compound | | | | |
|---|---|---|---|---|---|---|
| No. | A | B | Compound C | Compound D | Target Compound | Yield |
| 003 | 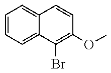 | 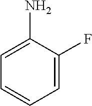 | 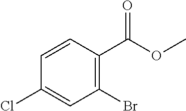 | 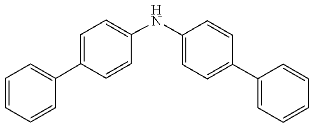 [SMILES: c1ccc(-c2ccc(Nc3ccc(-c4ccccc4)cc3)cc2)cc1] | 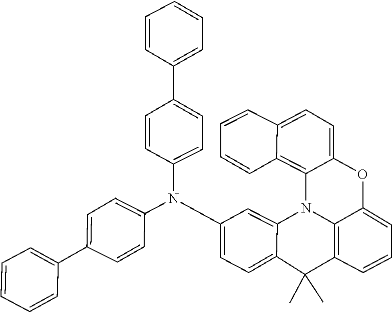 [SMILES: CC1(C)c2ccc(N(c3ccc(-c4ccccc4)cc3)c3ccc(-c4ccccc4)cc3)cc2N2c3c(cccc31)Oc1ccc3ccccc3c12] | 68% |
| 005 | 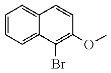 | 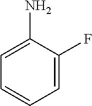 | 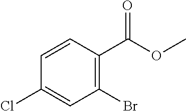 | 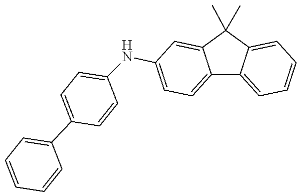 [SMILES: CC1(C)c2ccccc2-c2ccc(Nc3ccc(-c4ccccc4)cc3)cc21] | 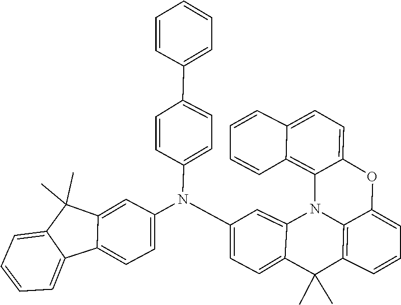 [SMILES: CC1(C)c2ccccc2-c2ccc(N(c3ccc(-c4ccccc4)cc3)c3ccc4c(c3)N3c5c(cccc5C4(C)C)Oc4ccc5ccccc5c43)cc21] | 72% |
| 011 | 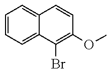 | 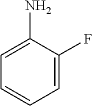 | 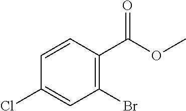 | 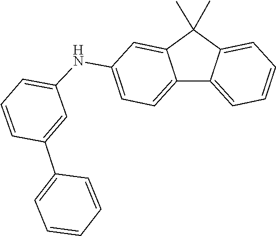 [SMILES: CC1(C)c2ccccc2-c2ccc(Nc3cccc(-c4ccccc4)c3)cc21] | 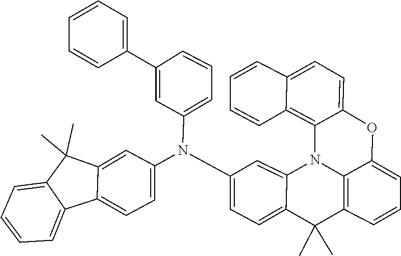 [SMILES: CC1(C)c2ccccc2-c2ccc(N(c3cccc(-c4ccccc4)c3)c3ccc4c(c3)N3c5c(cccc5C4(C)C)Oc4ccc5ccccc5c43)cc21] | 79% |
| 030 | 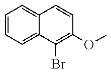 | 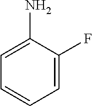 | 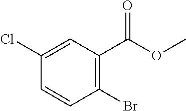 | 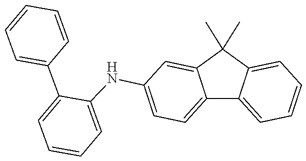 [SMILES: CC1(C)c2ccccc2-c2ccc(Nc3ccccc3-c3ccccc3)cc21] | 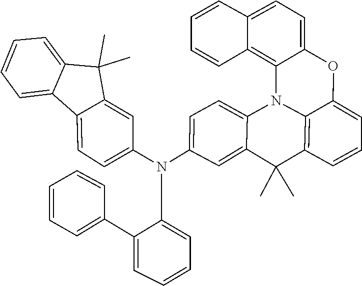 [SMILES: CC1(C)c2ccccc2-c2ccc(N(c3ccc4c(c3)C(C)(C)c3cccc5c3N4c3c(ccc4ccccc34)O5)c3ccccc3-c3ccccc3)cc21] | 69% |
| 031 | 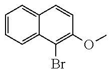 | 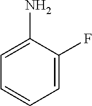 | 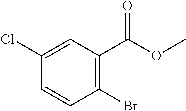 | 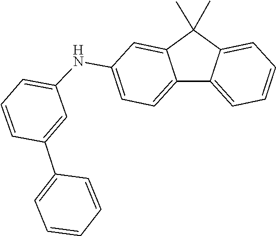 [SMILES: CC1(C)c2ccccc2-c2ccc(Nc3cccc(-c4ccccc4)c3)cc21] | 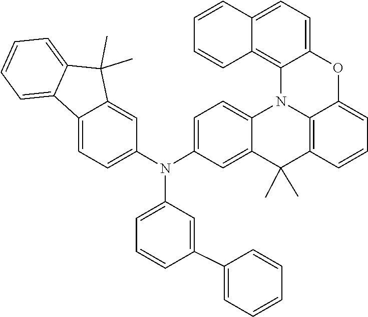 [SMILES: CC1(C)c2ccccc2-c2ccc(N(c3cccc(-c4ccccc4)c3)c3ccc4c(c3)C(C)(C)c3cccc5c3N4c3c(ccc4ccccc34)O5)cc21] | 81% |
| 039 | 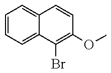 | 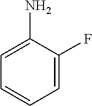 | 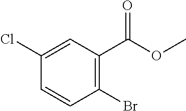 | 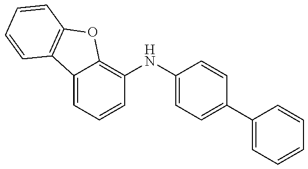 [SMILES: c1ccc(-c2ccc(Nc3cccc4c3oc3ccccc34)cc2)cc1] | 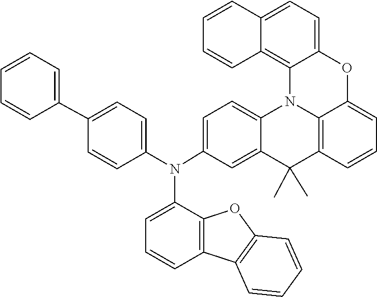 [SMILES: CC1(C)c2cc(N(c3ccc(-c4ccccc4)cc3)c3cccc4c3oc3ccccc34)ccc2N2c3c(cccc31)Oc1ccc3ccccc3c12] | 73% |
| 044 | 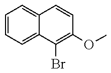 | 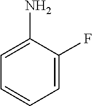 | 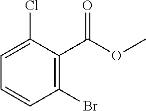 | 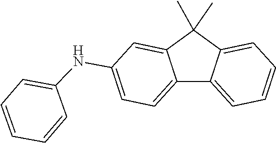 [SMILES: CC1(C)c2ccccc2-c2ccc(Nc3ccccc3)cc21] | 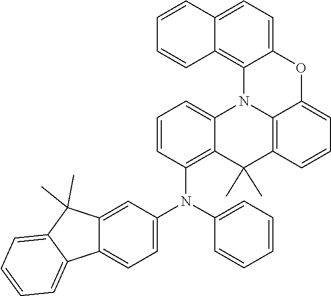 [SMILES: CC1(C)c2ccccc2-c2ccc(N(c3ccccc3)c3cccc4c3C(C)(C)c3cccc5c3N4c3c(ccc4ccccc34)O5)cc21] | 78% |
| 046 | 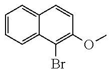 | 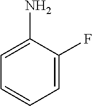 | 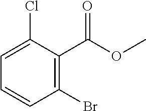 | 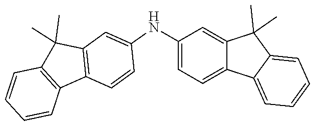 [SMILES: CC1(C)c2ccccc2-c2ccc(Nc3ccc4c(c3)C(C)(C)c3ccccc3-4)cc21] | 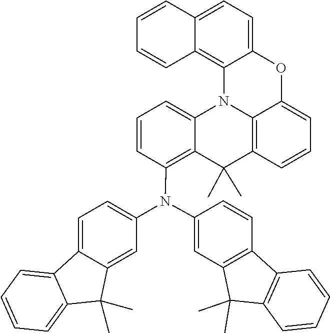 [SMILES: CC1(C)c2ccccc2-c2ccc(N(c3ccc4c(c3)C(C)(C)c3ccccc3-4)c3cccc4c3C(C)(C)c3cccc5c3N4c3c(ccc4ccccc34)O5)cc21] | 70% |
| 054 | 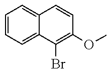 | 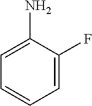 | 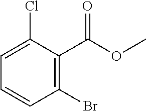 | 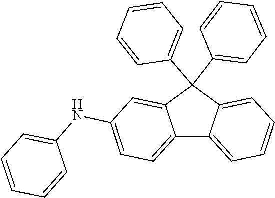 [SMILES: c1ccc(Nc2ccc3c(c2)C(c2ccccc2)(c2ccccc2)c2ccccc2-3)cc1] | 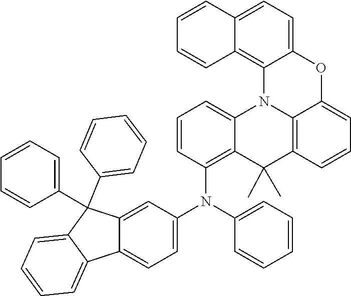 [SMILES: CC1(C)c2cccc3c2N(c2cccc(N(c4ccccc4)c4ccc5c(c4)C(c4ccccc4)(c4ccccc4)c4ccccc4-5)c21)c1c(ccc2ccccc12)O3] | 77% |
| 075 | 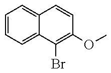 | 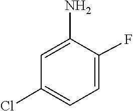 | 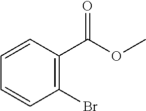 | 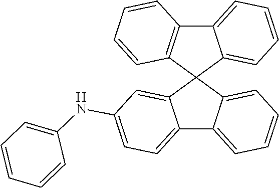 [SMILES: c1ccc(Nc2ccc3c(c2)C2(c4ccccc4-c4ccccc42)c2ccccc2-3)cc1] | 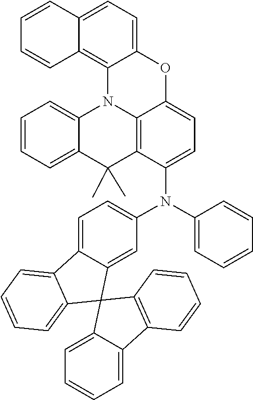 [SMILES: CC1(C)c2ccccc2N2c3c(ccc(N(c4ccccc4)c4ccc5c(c4)C4(c6ccccc6-c6ccccc64)c4ccccc4-5)c31)Oc1ccc3ccccc3c12] | 65% |
| 077 | 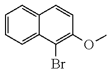 | 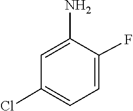 | 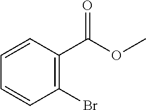 | 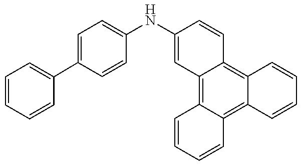 [SMILES: c1ccc(-c2ccc(Nc3ccc4c5ccccc5c5ccccc5c4c3)cc2)cc1] | 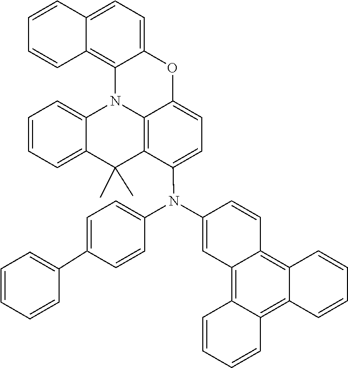 [SMILES: CC1(C)c2ccccc2N2c3c(ccc(N(c4ccc(-c5ccccc5)cc4)c4ccc5c6ccccc6c6ccccc6c5c4)c31)Oc1ccc3ccccc3c12] | 70% |
| 083 | 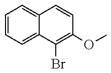 | 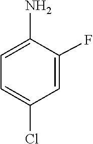 | 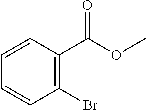 | 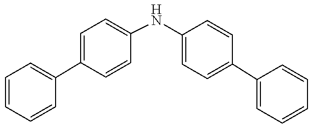 [SMILES: c1ccc(-c2ccc(Nc3ccc(-c4ccccc4)cc3)cc2)cc1] | 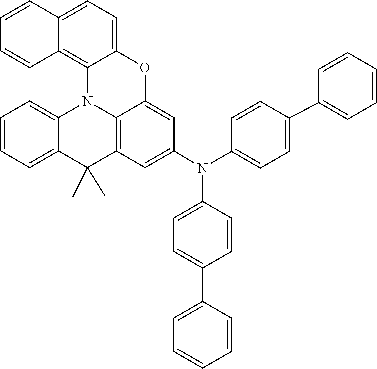 [SMILES: CC1(C)c2ccccc2N2c3c(cc(N(c4ccc(-c5ccccc5)cc4)c4ccc(-c5ccccc5)cc4)cc31)Oc1ccc3ccccc3c12] | 67% |
| 087 | 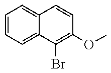 | 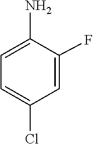 | 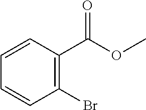 | 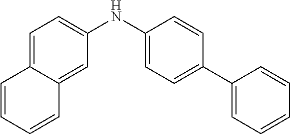 [SMILES: c1ccc(-c2ccc(Nc3ccc4ccccc4c3)cc2)cc1] | 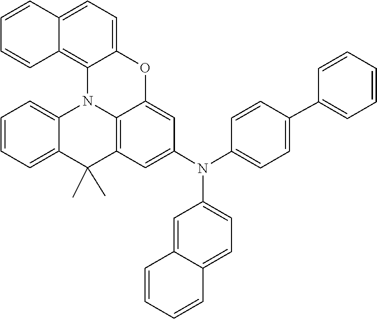 [SMILES: CC1(C)c2ccccc2N2c3c(cc(N(c4ccc(-c5ccccc5)cc4)c4ccc5ccccc5c4)cc31)Oc1ccc3ccccc3c12] | 73% |
| 098 | 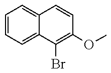 | 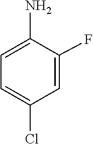 | 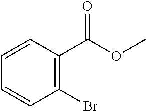 | 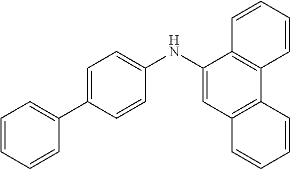 [SMILES: c1ccc(-c2ccc(Nc3cc4ccccc4c4ccccc34)cc2)cc1] | 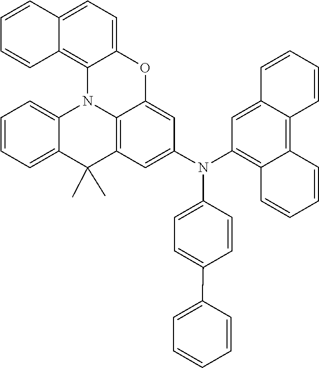 [SMILES: CC1(C)c2ccccc2N2c3c(cc(N(c4ccc(-c5ccccc5)cc4)c4cc5ccccc5c5ccccc45)cc31)Oc1ccc3ccccc3c12] | 71% |
| 108 | 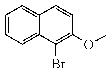 | 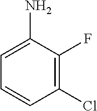 | 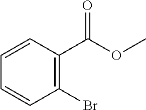 | 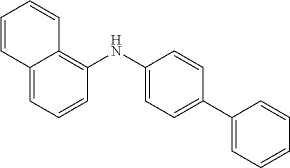 [SMILES: c1ccc(-c2ccc(Nc3cccc4ccccc34)cc2)cc1] | 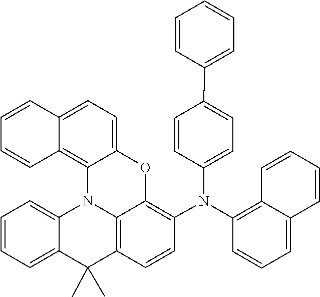 [SMILES: CC1(C)c2ccccc2N2c3c1ccc(N(c1ccc(-c4ccccc4)cc1)c1cccc4ccccc14)c3Oc1ccc3ccccc3c12] | 72% |
| 112 | 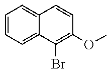 | 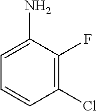 | 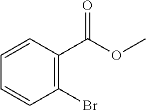 | 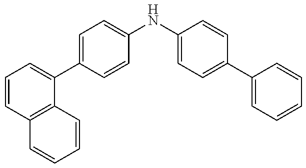 [SMILES: c1ccc(-c2ccc(Nc3ccc(-c4cccc5ccccc45)cc3)cc2)cc1] | 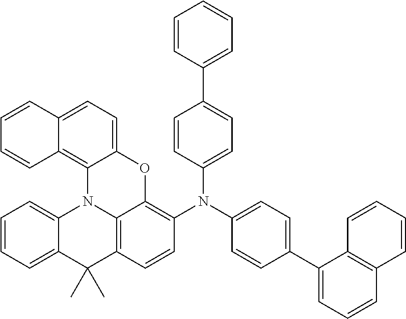 [SMILES: CC1(C)c2ccccc2N2c3c1ccc(N(c1ccc(-c4ccccc4)cc1)c1ccc(-c4cccc5ccccc45)cc1)c3Oc1ccc3ccccc3c12] | 76% |
| 119 | 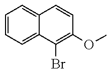 | 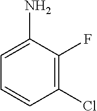 | 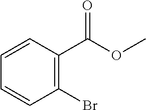 | 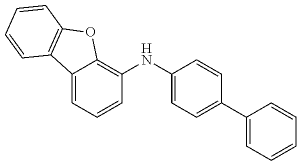 [SMILES: c1ccc(-c2ccc(Nc3cccc4c3oc3ccccc34)cc2)cc1] | 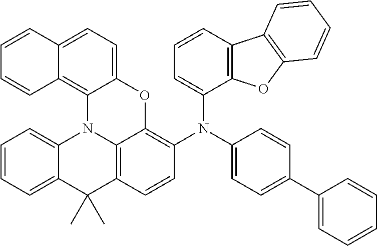 [SMILES: CC1(C)c2ccccc2N2c3c1ccc(N(c1ccc(-c4ccccc4)cc1)c1cccc4c1oc1ccccc14)c3Oc1ccc3ccccc3c12] | 78% |
| 131 | 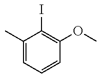 | 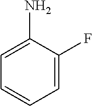 | 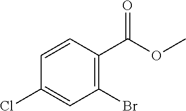 | 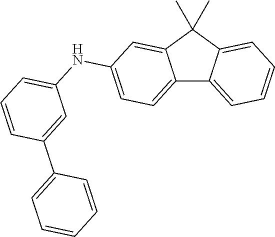 [SMILES: CC1(C)c2ccccc2-c2ccc(Nc3cccc(-c4ccccc4)c3)cc21] | 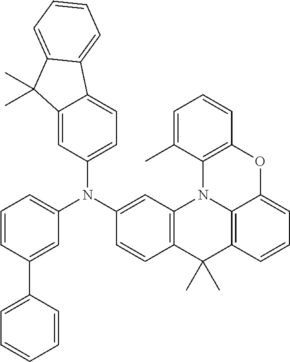 [SMILES: Cc1cccc2c1N1c3cc(N(c4cccc(-c5ccccc5)c4)c4ccc5c(c4)C(C)(C)c4ccccc4-5)ccc3C(C)(C)c3cccc(c31)O2] | 68% |
| 137 | 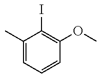 | 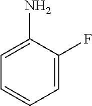 | 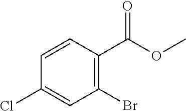 | 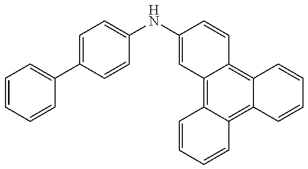 [SMILES: c1ccc(-c2ccc(Nc3ccc4c5ccccc5c5ccccc5c4c3)cc2)cc1] | 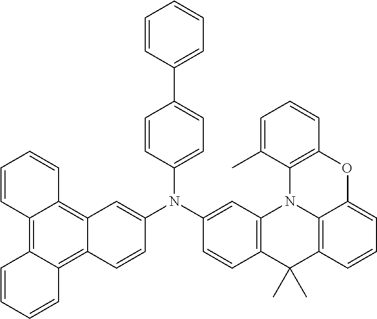 [SMILES: Cc1cccc2c1N1c3cc(N(c4ccc(-c5ccccc5)cc4)c4ccc5c6ccccc6c6ccccc6c5c4)ccc3C(C)(C)c3cccc(c31)O2] | 67% |
| 142 | 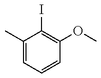 | 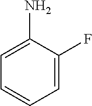 | 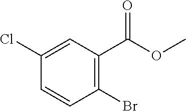 | 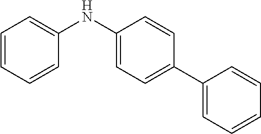 [SMILES: c1ccc(Nc2ccc(-c3ccccc3)cc2)cc1] | 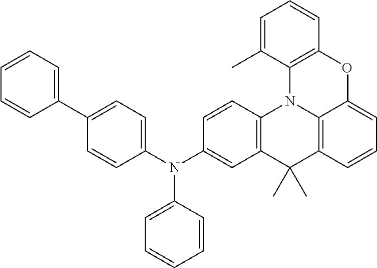 [SMILES: Cc1cccc2c1N1c3ccc(N(c4ccccc4)c4ccc(-c5ccccc5)cc4)cc3C(C)(C)c3cccc(c31)O2] | 72% |
| 144 | 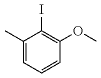 | 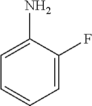 | 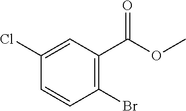 | 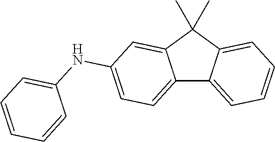 [SMILES: CC1(C)c2ccccc2-c2ccc(Nc3ccccc3)cc21] | 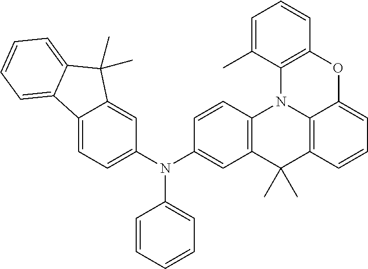 [SMILES: Cc1cccc2c1N1c3ccc(N(c4ccccc4)c4ccc5c(c4)C(C)(C)c4ccccc4-5)cc3C(C)(C)c3cccc(c31)O2] | 78% |
| 163 | 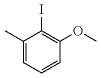 | 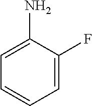 | 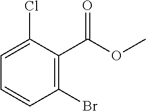 | 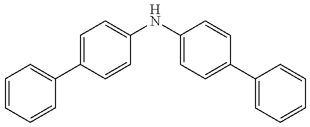 [SMILES: c1ccc(-c2ccc(Nc3ccc(-c4ccccc4)cc3)cc2)cc1] | 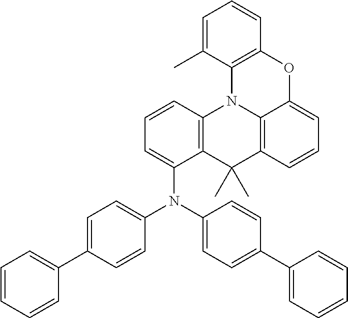 [SMILES: Cc1cccc2c1N1c3cccc(N(c4ccc(-c5ccccc5)cc4)c4ccc(-c5ccccc5)cc4)c3C(C)(C)c3cccc(c31)O2] | 69% |
| 165 | 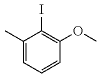 | 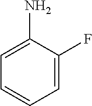 | 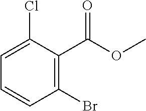 | 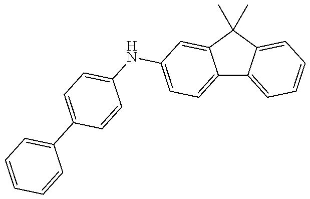 [SMILES: CC1(C)c2ccccc2-c2ccc(Nc3ccc(-c4ccccc4)cc3)cc21] | 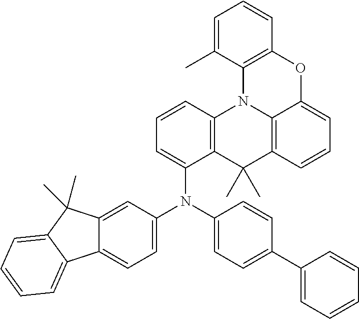 [SMILES: Cc1cccc2c1N1c3cccc(N(c4ccc(-c5ccccc5)cc4)c4ccc5c(c4)C(C)(C)c4ccccc4-5)c3C(C)(C)c3cccc(c31)O2] | 65% |
| 170 | 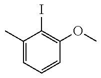 | 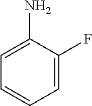 | 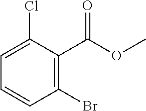 | 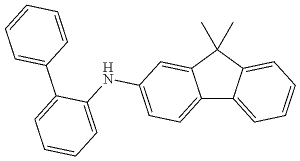 [SMILES: CC1(C)c2ccccc2-c2ccc(Nc3ccccc3-c3ccccc3)cc21] | 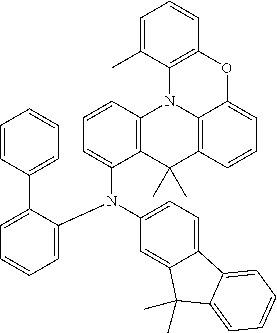 [SMILES: Cc1cccc2c1N1c3cccc(N(c4ccc5c(c4)C(C)(C)c4ccccc4-5)c4ccccc4-c4ccccc4)c3C(C)(C)c3cccc(c31)O2] | 75% |
| 196 | 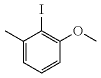 | 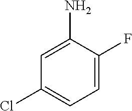 | 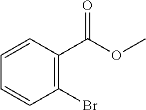 | 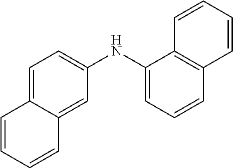 [SMILES: c1ccc2cc(Nc3cccc4ccccc34)ccc2c1] | 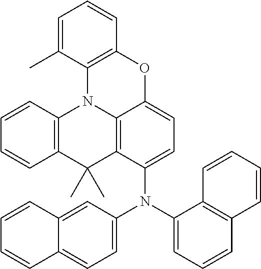 [SMILES: Cc1cccc2c1N1c3ccccc3C(C)(C)c3c(N(c4ccc5ccccc5c4)c4cccc5ccccc45)ccc(c31)O2] | 74% |
| 202 | 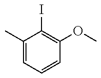 | 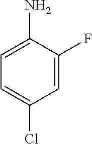 | 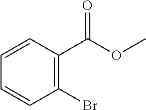 | 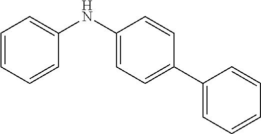 [SMILES: c1ccc(Nc2ccc(-c3ccccc3)cc2)cc1] | 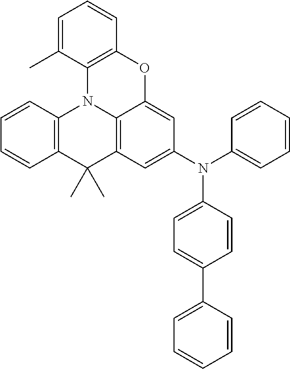 [SMILES: Cc1cccc2c1N1c3ccccc3C(C)(C)c3cc(N(c4ccccc4)c4ccc(-c5ccccc5)cc4)cc(c31)O2] | 75% |
| 204 | 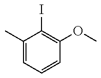 | 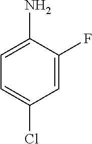 | 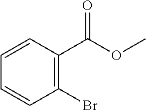 | 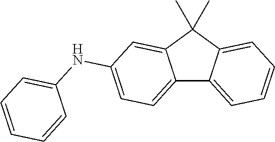 [SMILES: CC1(C)c2ccccc2-c2ccc(Nc3ccccc3)cc21] | 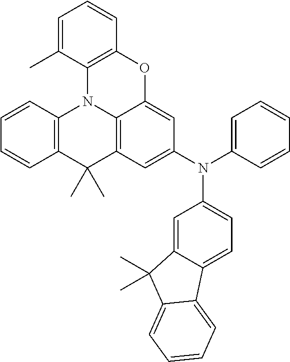 [SMILES: Cc1cccc2c1N1c3ccccc3C(C)(C)c3cc(N(c4ccccc4)c4ccc5c(c4)C(C)(C)c4ccccc4-5)cc(c31)O2] | 71% |
| 205 | 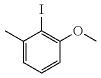 | 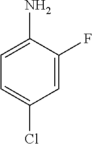 | 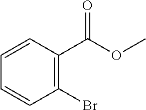 | 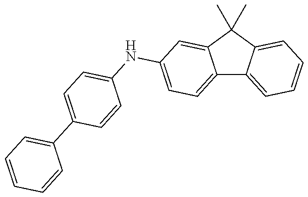 [SMILES: CC1(C)c2ccccc2-c2ccc(Nc3ccc(-c4ccccc4)cc3)cc21] | 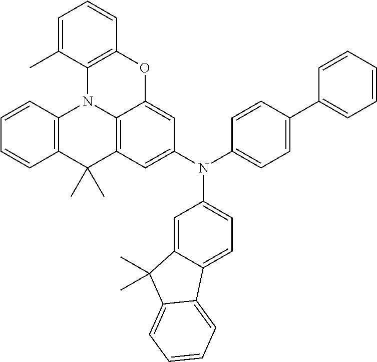 [SMILES: Cc1cccc2c1N1c3ccccc3C(C)(C)c3cc(N(c4ccc(-c5ccccc5)cc4)c4ccc5c(c4)C(C)(C)c4ccccc4-5)cc(c31)O2] | 79% |
| 212 | 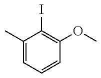 | 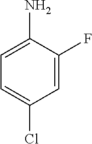 | 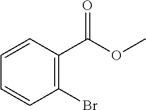 | 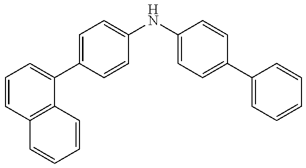 [SMILES: c1ccc(-c2ccc(Nc3ccc(-c4cccc5ccccc45)cc3)cc2)cc1] | 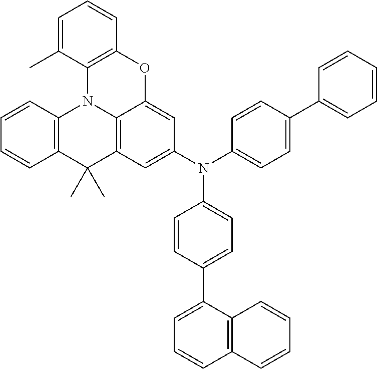 [SMILES: Cc1cccc2c1N1c3ccccc3C(C)(C)c3cc(N(c4ccc(-c5ccccc5)cc4)c4ccc(-c5cccc6ccccc56)cc4)cc(c31)O2] | 81% |
| 234 | 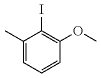 | 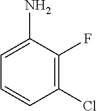 | 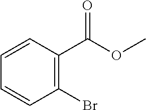 | 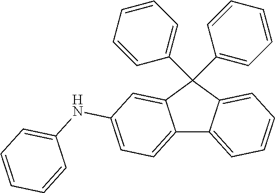 [SMILES: c1ccc(Nc2ccc3c(c2)C(c2ccccc2)(c2ccccc2)c2ccccc2-3)cc1] | 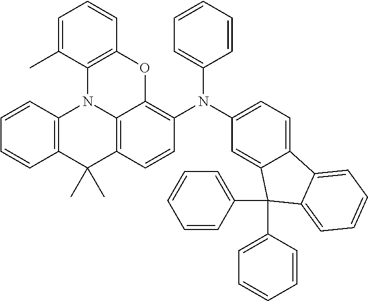 [SMILES: Cc1cccc2c1N1c3ccccc3C(C)(C)c3ccc(N(c4ccccc4)c4ccc5c(c4)C(c4ccccc4)(c4ccccc4)c4ccccc4-5)c(c31)O2] | 73% |
| 239 | 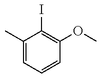 | 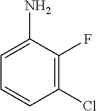 | 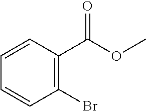 | 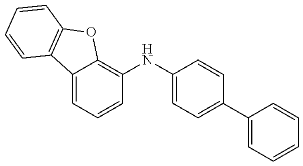 [SMILES: c1ccc(-c2ccc(Nc3cccc4c3oc3ccccc34)cc2)cc1] | 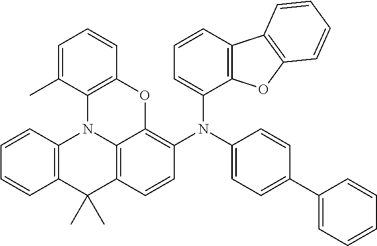 [SMILES: Cc1cccc2c1N1c3ccccc3C(C)(C)c3ccc(N(c4ccc(-c5ccccc5)cc4)c4cccc5c4oc4ccccc45)c(c31)O2] | 65% |
| 245 | 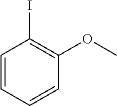 | 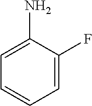 | 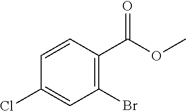 | 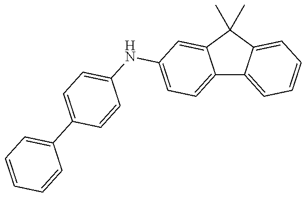 [SMILES: CC1(C)c2ccccc2-c2ccc(Nc3ccc(-c4ccccc4)cc3)cc21] | 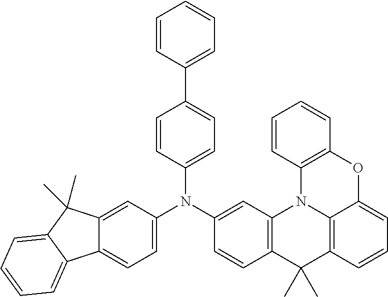 [SMILES: CC1(C)c2ccccc2-c2ccc(N(c3ccc(-c4ccccc4)cc3)c3ccc4c(c3)N3c5ccccc5Oc5cccc(c53)C4(C)C)cc21] | 74% |
| 247 | 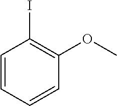 | 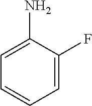 | 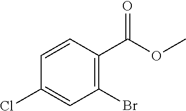 | 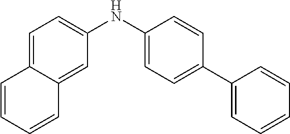 [SMILES: c1ccc(-c2ccc(Nc3ccc4ccccc4c3)cc2)cc1] | 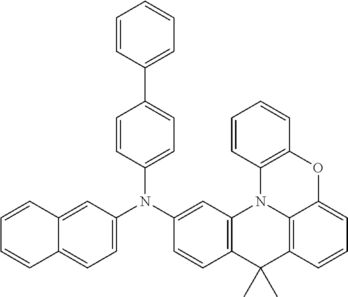 [SMILES: CC1(C)c2ccc(N(c3ccc(-c4ccccc4)cc3)c3ccc4ccccc4c3)cc2N2c3ccccc3Oc3cccc1c32] | 71% |
| 253 | 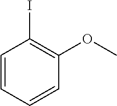 | 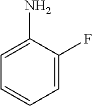 | 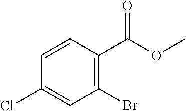 | 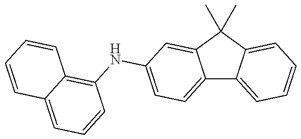 [SMILES: CC1(C)c2ccccc2-c2ccc(Nc3cccc4ccccc34)cc21] | 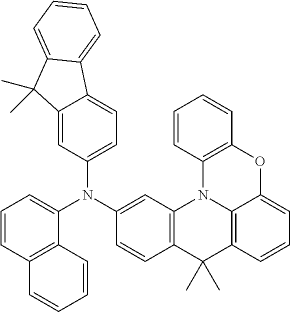 [SMILES: CC1(C)c2ccccc2-c2ccc(N(c3ccc4c(c3)N3c5ccccc5Oc5cccc(c53)C4(C)C)c3cccc4ccccc34)cc21] | 77% |
| 260 | 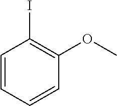 | 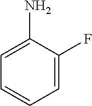 | 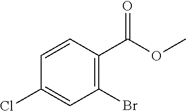 | 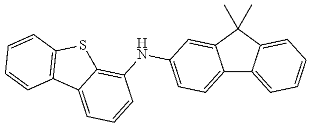 [SMILES: CC1(C)c2ccccc2-c2ccc(Nc3cccc4c3sc3ccccc34)cc21] | 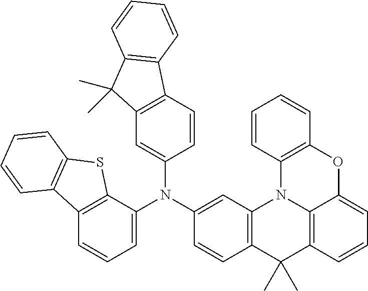 [SMILES: CC1(C)c2ccccc2-c2ccc(N(c3ccc4c(c3)N3c5ccccc5Oc5cccc(c53)C4(C)C)c3cccc4c3sc3ccccc34)cc21] | 77% |
| 269 | 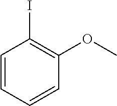 | 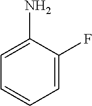 | 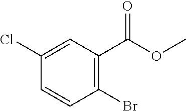 | 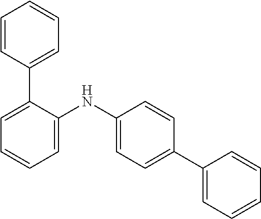 [SMILES: c1ccc(-c2ccc(Nc3ccccc3-c3ccccc3)cc2)cc1] | 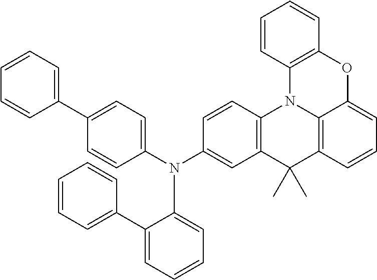 [SMILES: CC1(C)c2cc(N(c3ccc(-c4ccccc4)cc3)c3ccccc3-c3ccccc3)ccc2N2c3ccccc3Oc3cccc1c32] | 72% |
| 275 | 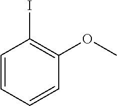 | 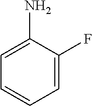 | 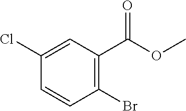 | 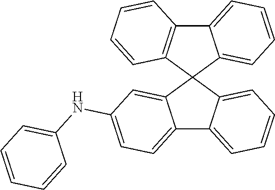 [SMILES: c1ccc(Nc2ccc3c(c2)C2(c4ccccc4-c4ccccc42)c2ccccc2-3)cc1] | 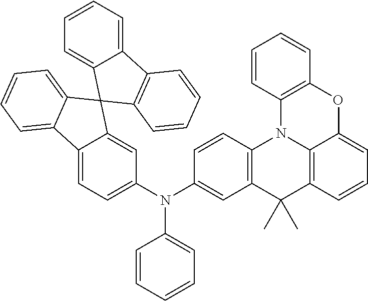 [SMILES: CC1(C)c2cc(N(c3ccccc3)c3ccc4c(c3)C3(c5ccccc5-c5ccccc53)c3ccccc3-4)ccc2N2c3ccccc3Oc3cccc1c32] | 78% |
| 290 | 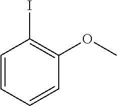 | 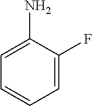 | 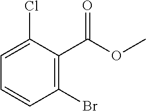 | 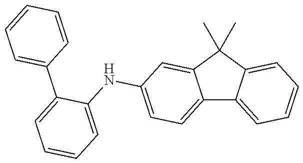 [SMILES: CC1(C)c2ccccc2-c2ccc(Nc3ccccc3-c3ccccc3)cc21] | 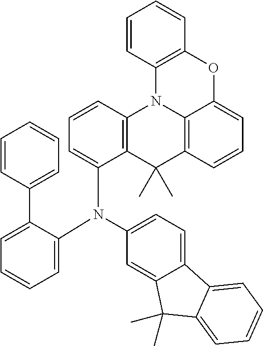 [SMILES: CC1(C)c2ccccc2-c2ccc(N(c3ccccc3-c3ccccc3)c3cccc4c3C(C)(C)c3cccc5c3N4c3ccccc3O5)cc21] | 76% |
| 302 | 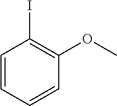 | 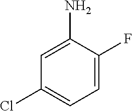 | 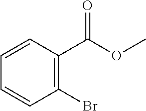 | 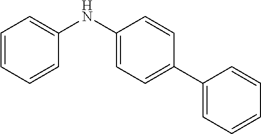 [SMILES: c1ccc(Nc2ccc(-c3ccccc3)cc2)cc1] | 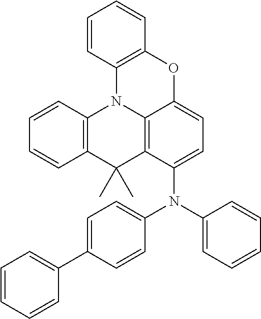 [SMILES: CC1(C)c2ccccc2N2c3ccccc3Oc3ccc(N(c4ccccc4)c4ccc(-c5ccccc5)cc4)c1c32] | 74% |
| 304 | 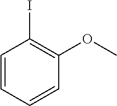 | 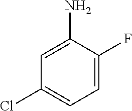 | 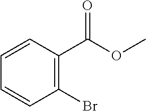 | 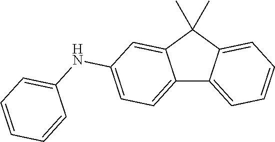 [SMILES: CC1(C)c2ccccc2-c2ccc(Nc3ccccc3)cc21] | 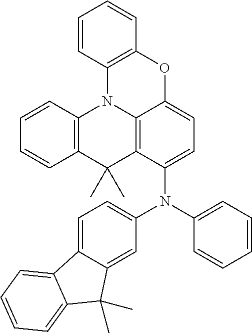 [SMILES: CC1(C)c2ccccc2-c2ccc(N(c3ccccc3)c3ccc4c5c3C(C)(C)c3ccccc3N5c3ccccc3O4)cc21] | 75% |
| 317 | 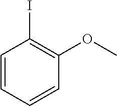 | 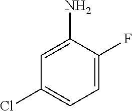 | 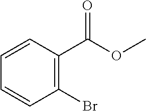 | 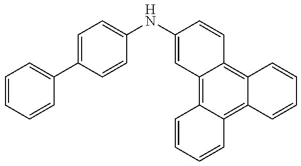 [SMILES: c1ccc(-c2ccc(Nc3ccc4c5ccccc5c5ccccc5c4c3)cc2)cc1] | 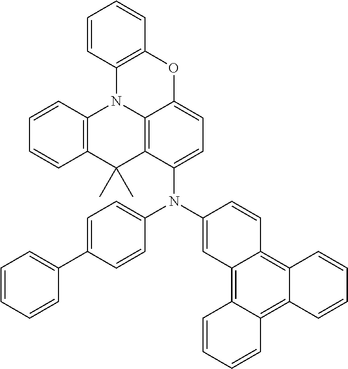 [SMILES: CC1(C)c2ccccc2N2c3ccccc3Oc3ccc(N(c4ccc(-c5ccccc5)cc4)c4ccc5c6ccccc6c6ccccc6c5c4)c1c32] | 72% |
| 323 | 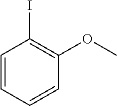 | 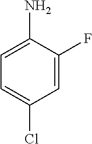 | 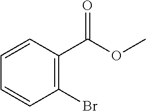 | 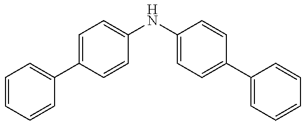 [SMILES: c1ccc(-c2ccc(Nc3ccc(-c4ccccc4)cc3)cc2)cc1] | 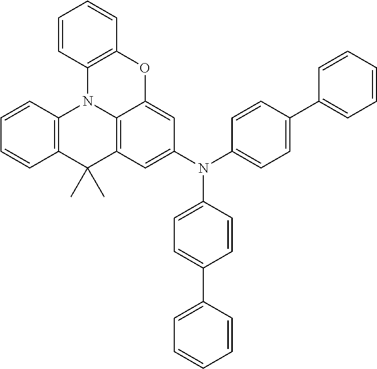 [SMILES: CC1(C)c2ccccc2N2c3ccccc3Oc3cc(N(c4ccc(-c5ccccc5)cc4)c4ccc(-c5ccccc5)cc4)cc1c32] | 68% |
| 338 | 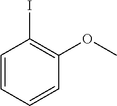 | 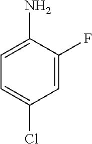 | 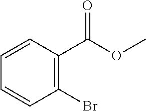 | 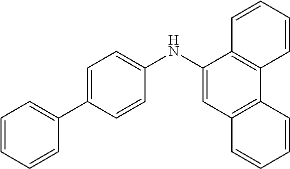 [SMILES: c1ccc(-c2ccc(Nc3cc4ccccc4c4ccccc34)cc2)cc1] | 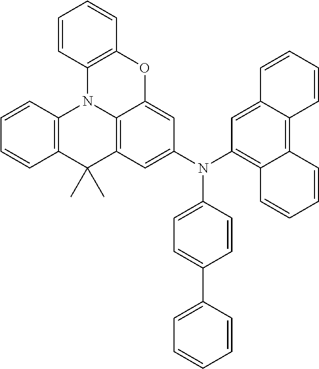 [SMILES: CC1(C)c2ccccc2N2c3ccccc3Oc3cc(N(c4ccc(-c5ccccc5)cc4)c4cc5ccccc5c5ccccc45)cc1c32] | 66% |
| 346 | 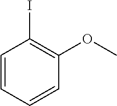 | 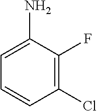 | 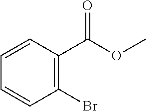 | 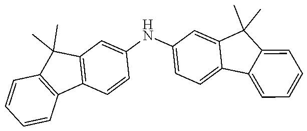 [SMILES: CC1(C)c2ccccc2-c2ccc(Nc3ccc4c(c3)C(C)(C)c3ccccc3-4)cc21] | 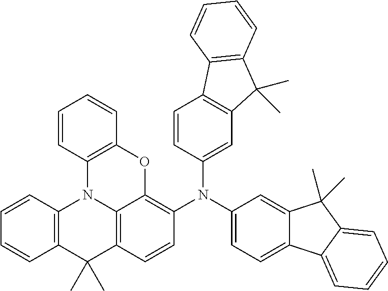 [SMILES: CC1(C)c2ccccc2-c2ccc(N(c3ccc4c(c3)C(C)(C)c3ccccc3-4)c3ccc4c5c3Oc3ccccc3N5c3ccccc3C4(C)C)cc21] | 69% |
| 357 | 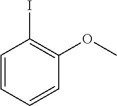 | 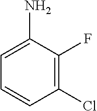 | 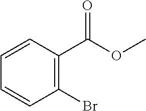 | 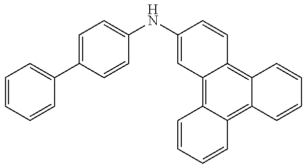 [SMILES: c1ccc(-c2ccc(Nc3ccc4c5ccccc5c5ccccc5c4c3)cc2)cc1] | 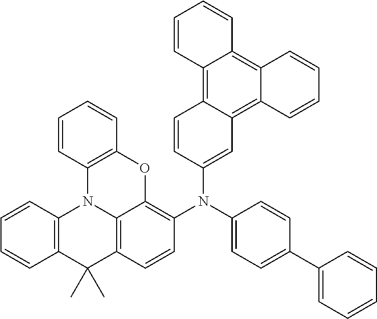 [SMILES: CC1(C)c2ccccc2N2c3ccccc3Oc3c(N(c4ccc(-c5ccccc5)cc4)c4ccc5c6ccccc6c6ccccc6c5c4)ccc1c32] | 73% |

### <Preparation Example 2> Preparation of Compound 426-P

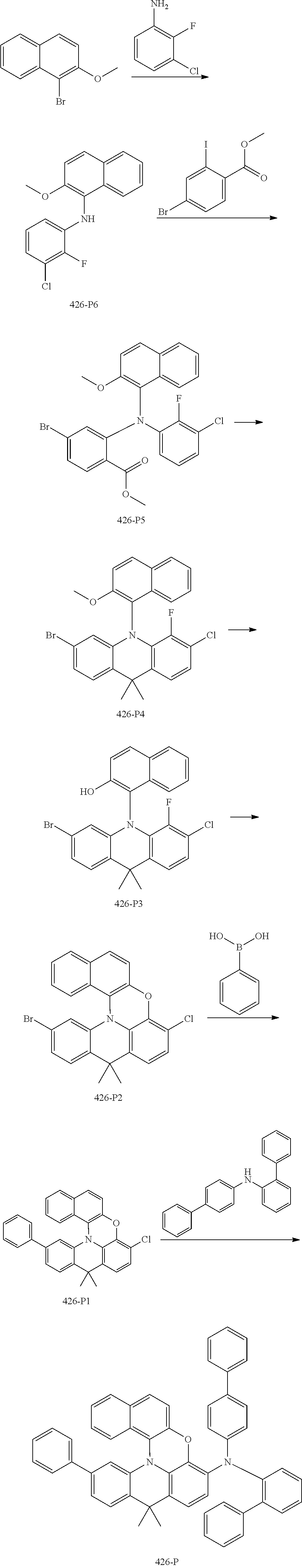

1) Preparation of Compound 426-P6

Dissolving 1-bromo-2-methoxynaphthalene (50 g, 210.89 mmol) and 3-cloro-2-fluoroaniline (39.91 g, 274.16 mmol) in toluene (500 ml), Pd(OAc)₂ (0.95 g, 4.22 mmol), xantphos (6.1 g, 10.54 mmol) and t-BuONa (40.53 g, 421.78 mmol) were introduced thereto, and the mixture was stirred under reflux for 2 hours. After the reaction was completed, dichloromethane was introduced to the reaction solution for dissolution. The result was extracted with distilled water, and after drying the organic layer with anhydrous MgSO₄, the solvent was removed using a rotary evaporator. The result was purified by column chromatography using dichloromethane and hexane as a developing solvent to obtain Compound 426-P6 (45 g, yield 71%).

2) Preparation of Compound 426-P5

Dissolving Compound 426-P6 (45 g, 149.14 mmol) and methyl 4-bromo-2-iodobenzoate (66.10 g, 193.88 mmol) in toluene (500 ml), Pd(OAc)₂ (0.67 g, 2.98 mmol), xantphos (4.31 g, 7.46 mmol) and t-BuONa (28.66 g, 298.27 mmol) were introduced thereto, and the mixture was stirred under reflux for 2 hours. After the reaction was completed, dichloromethane was introduced to the reaction solution for dissolution. The result was extracted with distilled water, and after drying the organic layer with anhydrous MgSO₄, the solvent was removed using a rotary evaporator. The result was purified by column chromatography using dichloromethane and hexane as a developing solvent to obtain Compound 426-P5 (57 g, yield 74%).

3) Preparation of Compound 426-P4

Dissolving Compound 426-P5 (57 g, 110.73 mmol) in tetrahydrofuran (500 ml), methylmagnesium bromide (3 M solution in ether) (110 ml, 332.19 mmol) was slowly added thereto at 0° C., and the mixture was stirred for 6 hours at 60° C. After the reaction was finished, water was added to the reaction solution to terminate the reaction. The result was extracted using dichloromethane and distilled water, and after drying the organic layer with anhydrous MgSO₄, the solvent was removed using a rotary evaporator. After that, the result was dissolved in dichloromethane, boron trifluoride diethyl etherate was added to the reaction material, and the mixture was stirred for 4 hours at room temperature. After the reaction was finished, the result was purified by column chromatography using dichloromethane and hexane as a developing solvent to obtain Compound 426-P4 (42 g, yield 76%).

4) Preparation of Compound 426-P3

Dissolving Compound 426-P4 (42 g, 84.54 mmol) in dichloromethane (400 ml), boron tribromide (31.77 g, 126.81 mmol) was slowly added thereto at 0° C., and the mixture was stirred for 3 hours. After the reaction was finished, distilled water was slowly added to the reaction solution to terminate the reaction.

The result was extracted with dichloromethane and distilled water, and after drying the organic layer with anhydrous MgSO₄, the solvent was removed using a rotary evaporator. The result was purified by column chromatography using dichloromethane and hexane as a developing solvent to obtain Compound 426-P3 (36 g, 88%).

5) Preparation of Compound 426-P2

Compound 426-P3 (36 g, 74.57 mmol) was dissolved in N,N-dimethylacetamide (400 ml), and after heating to 150° C., Cs₂CO₃ (48.59 g, 149.14 mmol) was introduced thereto, and the mixture was stirred under reflux for 30 minutes. After the reaction was completed, the result was extracted with dichloromethane and distilled water, and after drying the organic layer with anhydrous MgSO₄, the solvent was removed using a rotary evaporator. The result was purified by column chromatography using dichloromethane and hexane as a developing solvent to obtain Compound 426-P2 (26 g, yield 75%).

6) Preparation of Compound 426-P1

Dissolving Compound 426-P2 (26 g, 56.18 mmol) and phenylboronic acid (7.19 g, 58.99 mmol) in toluene (300 ml), ethanol (60 ml) and distilled water (60 ml), tetrakis(triphenylphosphine)palladium(0) (Pd(PPh₃)₄ (1.30 g, 1.12 mmol) and K₂CO₃ (19.41 g, 140.46 mmol) were introduced thereto, and the mixture was stirred under reflux for 12 hours. After the reaction was completed, the reaction solution was extracted with dichloromethane and distilled water, and after drying the organic layer with anhydrous MgSO₄, the solvent was removed using a rotary evaporator. The result was purified by column chromatography using dichloromethane and hexane as a developing solvent to obtain Compound 426-P1 (22 g, yield 85%).

7) Preparation of Compound 426-P

Dissolving Compound 426-P1 (10 g, 21.74 mmol) and N-([1,1′-biphenyl]-4-yl)-[1,1′-biphenyl]-2-amine (6.99 g, 21.74 mmol) in toluene (100 ml), tris(dibenzylideneacetone)dipalladium (Pd₂(dba)₃) (0.40 g, 0.43 mmol), Xphos (0.52 g, 1.09 mmol) and t-BuONa (4.18 g, 43.48 mmol) were introduced thereto, and the mixture was stirred under reflux for 2 hours. After the reaction was completed, dichloromethane was introduced to the reaction solution for dissolution. The result was extracted with distilled water, and after drying the organic layer with anhydrous MgSO₄, the solvent was removed using a rotary evaporator. The result was purified by column chromatography using dichloromethane and hexane as a developing solvent to obtain Compound 426 (12 g, yield 74%).

The following target compounds were synthesized in the same manner as in Preparation Example 2 except that Compound E was used instead of 1-bromo-2-methoxynaphthalene, Compound F was used instead of 3-chloro-2-fluoroaniline, Compound G was used instead of methyl 4-bromo-2-iodobenzoate, Compound H was used instead of phenylboronic acid, and Compound I was used instead of N-([1,1′-biphenyl]-4-yl)-[1,1′-biphenyl]-2-amine.

TABLE 2

| | Compound | Compound | Compound | Compound |
|---|---|---|---|---|
| No. | E | F | G | H |
| 362 | 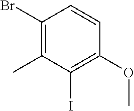 | 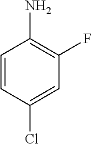 | 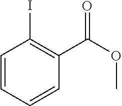 | 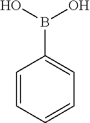 |
| 372 | 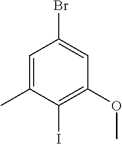 | 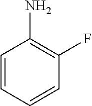 | 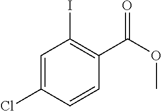 | 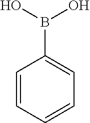 |
| 383 | 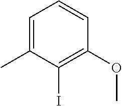 | 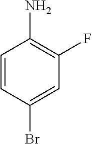 | 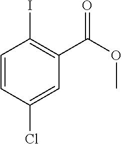 | 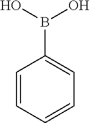 |
| 405 | 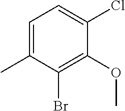 | 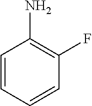 | 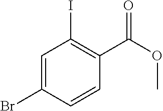 | 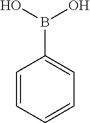 |
| 412 | 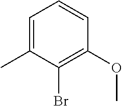 | 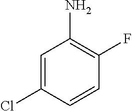 | 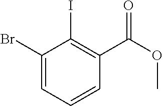 | 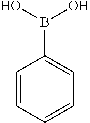 |
| 449 | 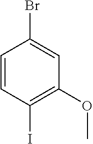 | 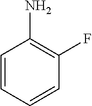 | 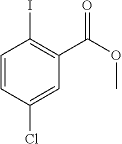 | 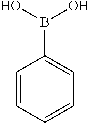 |
| 471 | 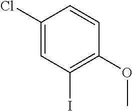 | 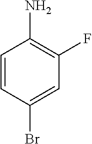 | 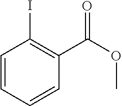 | 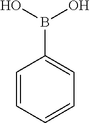 |
| 479 | 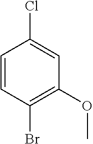 | 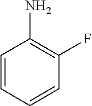 | 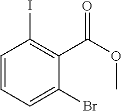 | 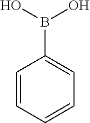 |
| 505 | 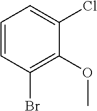 | 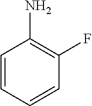 | 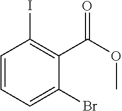 | 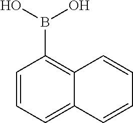 |
| 519 | 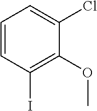 | 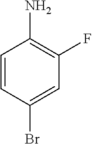 | 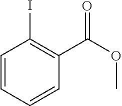 | 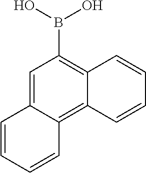 |

| No. | Compound I | Target Compound | Yield |
|---|---|---|---|
| 362 | 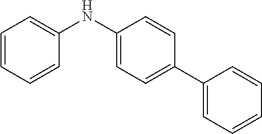 [SMILES: c1ccc(Nc2ccc(-c3ccccc3)cc2)cc1] | 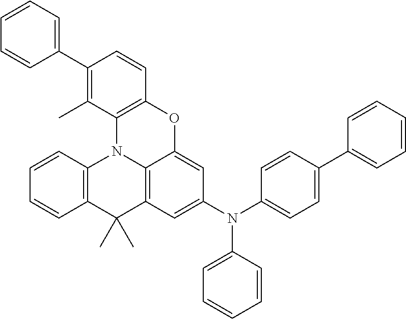 [SMILES: Cc1c(-c2ccccc2)ccc2c1N1c3ccccc3C(C)(C)c3cc(N(c4ccccc4)c4ccc(-c5ccccc5)cc4)cc(c31)O2] | 77% |
| 372 | 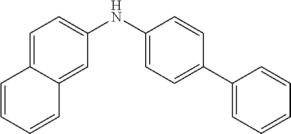 [SMILES: c1ccc(-c2ccc(Nc3ccc4ccccc4c3)cc2)cc1] | 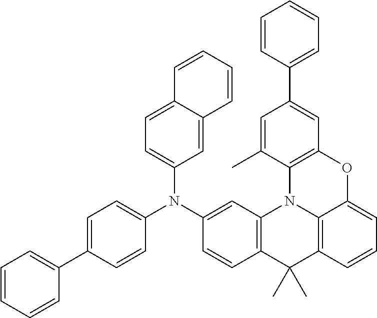 [SMILES: Cc1cc(-c2ccccc2)cc2c1N1c3cc(N(c4ccc(-c5ccccc5)cc4)c4ccc5ccccc5c4)ccc3C(C)(C)c3cccc(c31)O2] | 73% |
| 383 | 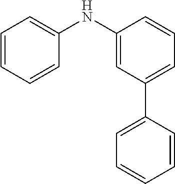 [SMILES: c1ccc(Nc2cccc(-c3ccccc3)c2)cc1] | 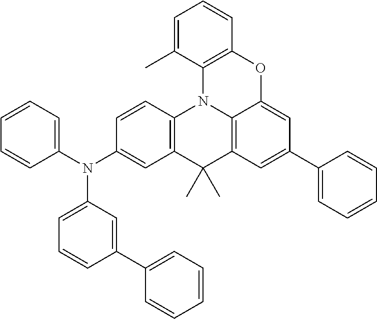 [SMILES: Cc1cccc2c1N1c3ccc(N(c4ccccc4)c4cccc(-c5ccccc5)c4)cc3C(C)(C)c3cc(-c4ccccc4)cc(c31)O2] | 78% |
| 405 | 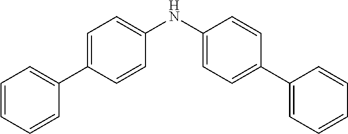 [SMILES: c1ccc(-c2ccc(Nc3ccc(-c4ccccc4)cc3)cc2)cc1] | 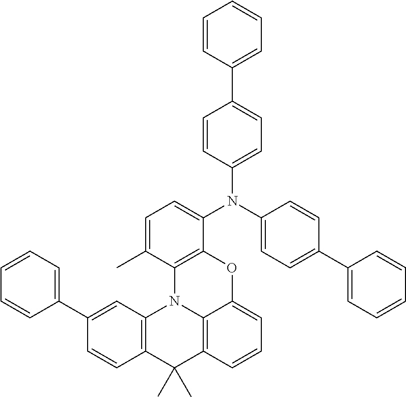 [SMILES: Cc1ccc(N(c2ccc(-c3ccccc3)cc2)c2ccc(-c3ccccc3)cc2)c2c1N1c3cc(-c4ccccc4)ccc3C(C)(C)c3cccc(c31)O2] | 74% |
| 412 | 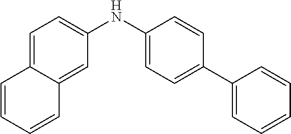 [SMILES: c1ccc(-c2ccc(Nc3ccc4ccccc4c3)cc2)cc1] | 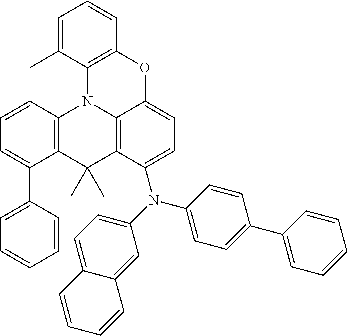 [SMILES: Cc1cccc2c1N1c3cccc(-c4ccccc4)c3C(C)(C)c3c(N(c4ccc(-c5ccccc5)cc4)c4ccc5ccccc5c4)ccc(c31)O2] | 71% |
| 449 | 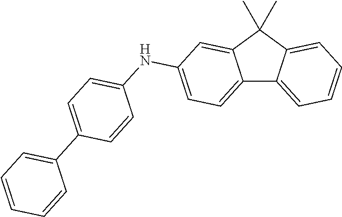 [SMILES: CC1(C)c2ccccc2-c2ccc(Nc3ccc(-c4ccccc4)cc3)cc21] | 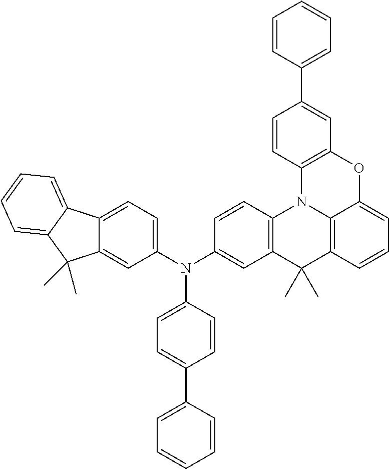 [SMILES: CC1(C)c2ccccc2-c2ccc(N(c3ccc(-c4ccccc4)cc3)c3ccc4c(c3)C(C)(C)c3cccc5c3N4c3ccc(-c4ccccc4)cc3O5)cc21] | 75% |
| 471 | 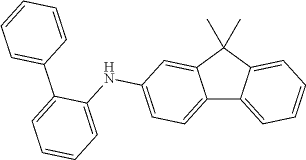 [SMILES: CC1(C)c2ccccc2-c2ccc(Nc3ccccc3-c3ccccc3)cc21] | 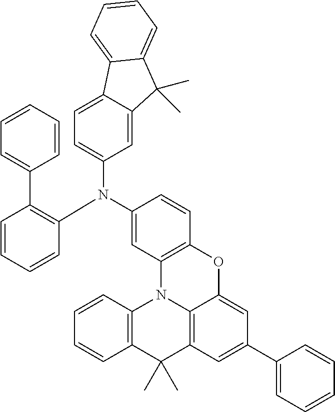 [SMILES: CC1(C)c2ccccc2-c2ccc(N(c3ccc4c(c3)N3c5ccccc5C(C)(C)c5cc(-c6ccccc6)cc(c53)O4)c3ccccc3-c3ccccc3)cc21] | 74% |
| 479 | 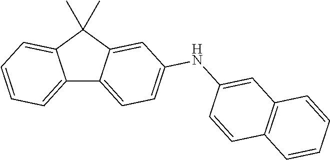 [SMILES: CC1(C)c2ccccc2-c2ccc(Nc3ccc4ccccc4c3)cc21] | 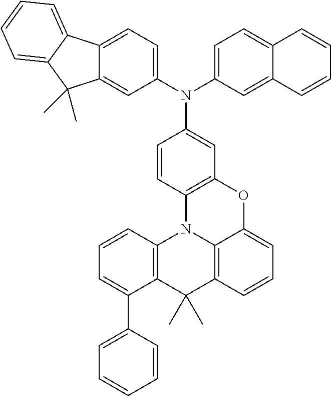 [SMILES: CC1(C)c2ccccc2-c2ccc(N(c3ccc4c(c3)Oc3cccc5c3N4c3cccc(-c4ccccc4)c3C5(C)C)c3ccc4ccccc4c3)cc21] | 68% |
| 505 | 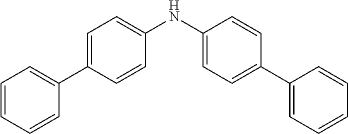 [SMILES: c1ccc(-c2ccc(Nc3ccc(-c4ccccc4)cc3)cc2)cc1] | 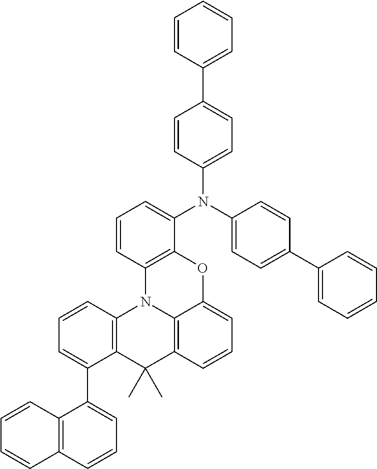 [SMILES: CC1(C)c2cccc3c2N(c2cccc(N(c4ccc(-c5ccccc5)cc4)c4ccc(-c5ccccc5)cc4)c2O3)c2cccc(-c3cccc4ccccc34)c21] | 66% |
| 519 | 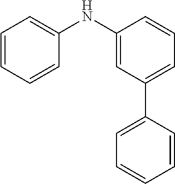 [SMILES: c1ccc(Nc2cccc(-c3ccccc3)c2)cc1] | 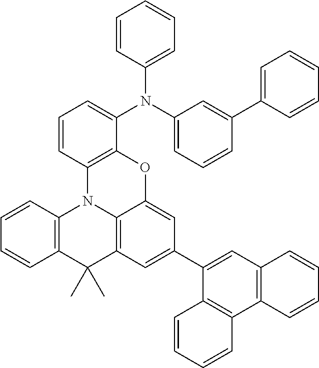 [SMILES: CC1(C)c2ccccc2N2c3cccc(N(c4ccccc4)c4cccc(-c5ccccc5)c4)c3Oc3cc(-c4cc5ccccc5c5ccccc45)cc1c32] | 72% |

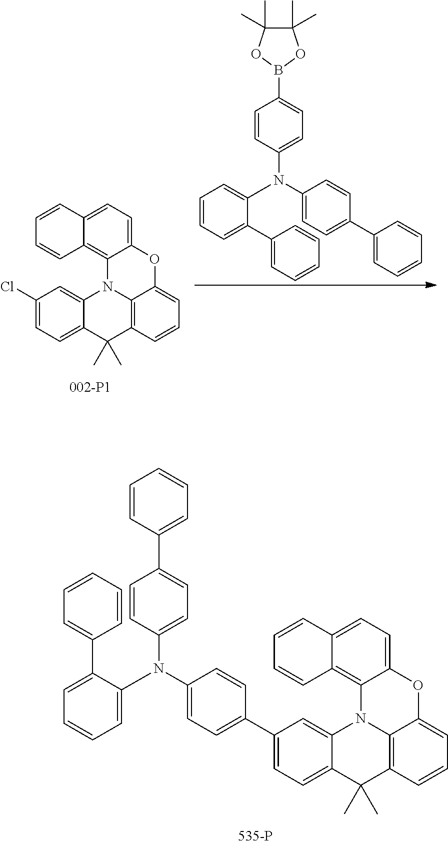 [SMILES: CC1(C)c2ccc(Cl)cc2N2c3c(cccc31)Oc1ccc3ccccc3c12 ; CC1(C)OB(c2ccc(N(c3ccc(-c4ccccc4)cc3)c3ccccc3-c3ccccc3)cc2)OC1(C)C ; CC1(C)c2ccc(-c3ccc(N(c4ccc(-c5ccccc5)cc4)c4ccccc4-c4ccccc4)cc3)cc2N2c3c(cccc31)Oc1ccc3ccccc3c12]

1) Preparation of Compound 535-P

Dissolving Compound 002-P1 (10 g, 26.05 mmol) and N-([1,1′-biphenyl]-4-yl)-N-(4-(4,4,5,5-tetramethyl-1,3,2-dioxaborolan-2-yl)phenyl)-[1,1′-biphenyl]-2-amine (14.32 g, 27.35 mmol) in 1,4-dioxane (100 ml) and distilled water (20 ml), bis(dibenzylideneacetone)palladium (Pd(dba)₂) (0.30 g, 0.52 mmol), Xphos (0.62 g, 1.30 mmol) and K₂CO₃ (9.00 g, 65.13 mmol) were introduced thereto, and the mixture was stirred under reflux for 12 hours. After the reaction was completed, the reaction solution was extracted with dichloromethane and distilled water, and after drying the organic layer with anhydrous MgSO₄, the solvent was removed using a rotary evaporator. The result was purified by column chromatography using dichloromethane and hexane as a developing solvent to obtain Compound 535 (15 g, yield 77%).

The following target compounds were synthesized in the same manner as in Preparation Example 3 except that Compound J was used instead of Compound 002-P1, and Compound K was used instead of N-([1,1′-biphenyl]-4-yl)-N-(4-(4,4,5,5-tetramethyl-1,3,2-dioxaborolan-2-yl)phenyl)-[1,1′-biphenyl]2-amine.

TABLE 3

| No. | Compound J | Compound K |
|---|---|---|
| 541 | 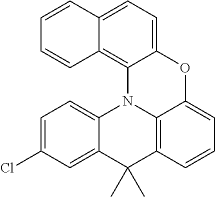 [SMILES: CC1(C)c2cc(Cl)ccc2N2c3c(cccc31)Oc1ccc3ccccc3c12] | 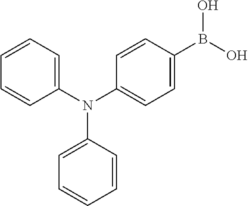 [SMILES: OB(O)c1ccc(N(c2ccccc2)c2ccccc2)cc1] |
| 545 | 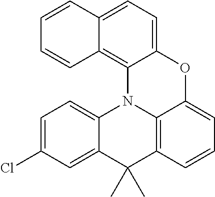 [SMILES: CC1(C)c2cc(Cl)ccc2N2c3c(cccc31)Oc1ccc3ccccc3c12] | 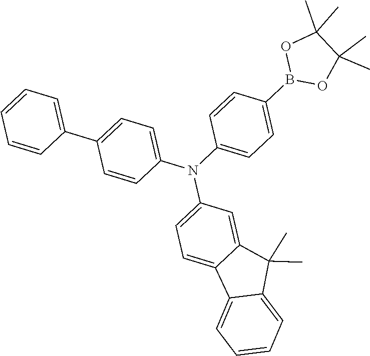 [SMILES: CC1(C)c2ccccc2-c2ccc(N(c3ccc(B4OC(C)(C)C(C)(C)O4)cc3)c3ccc(-c4ccccc4)cc3)cc21] |
| 603 | 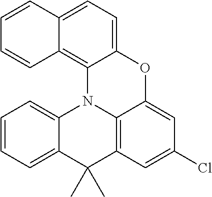 [SMILES: CC1(C)c2ccccc2N2c3c(cc(Cl)cc31)Oc1ccc3ccccc3c12] | 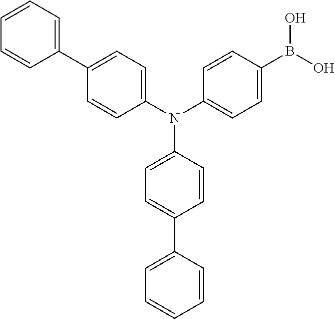 [SMILES: OB(O)c1ccc(N(c2ccc(-c3ccccc3)cc2)c2ccc(-c3ccccc3)cc2)cc1] |
| 622 | 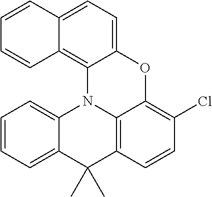 [SMILES: CC1(C)c2ccccc2N2c3c1ccc(Cl)c3Oc1ccc3ccccc3c12] | 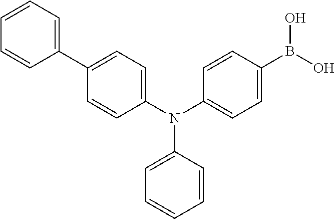 [SMILES: OB(O)c1ccc(N(c2ccccc2)c2ccc(-c3ccccc3)cc2)cc1] |
| 651 | 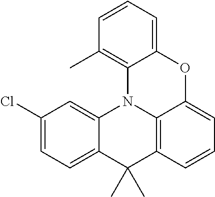 [SMILES: Cc1cccc2c1N1c3cc(Cl)ccc3C(C)(C)c3cccc(c31)O2] | 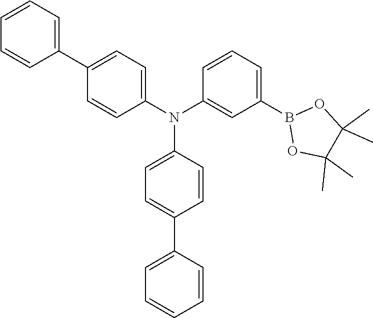 [SMILES: CC1(C)OB(c2cccc(N(c3ccc(-c4ccccc4)cc3)c3ccc(-c4ccccc4)cc3)c2)OC1(C)C] |
| 663 | 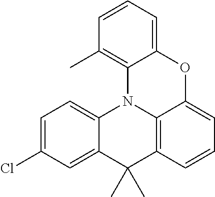 [SMILES: Cc1cccc2c1N1c3ccc(Cl)cc3C(C)(C)c3cccc(c31)O2] | 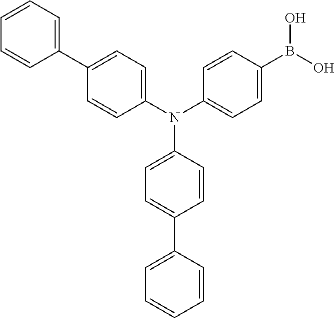 [SMILES: OB(O)c1ccc(N(c2ccc(-c3ccccc3)cc2)c2ccc(-c3ccccc3)cc2)cc1] |
| 685 | 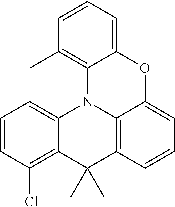 [SMILES: Cc1cccc2c1N1c3cccc(Cl)c3C(C)(C)c3cccc(c31)O2] | 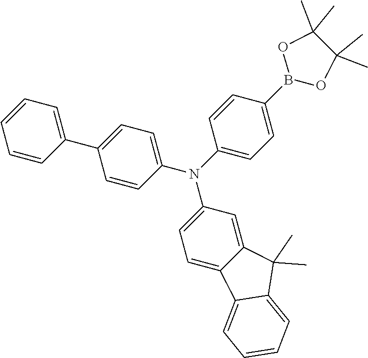 [SMILES: CC1(C)c2ccccc2-c2ccc(N(c3ccc(B4OC(C)(C)C(C)(C)O4)cc3)c3ccc(-c4ccccc4)cc3)cc21] |
| 762 | 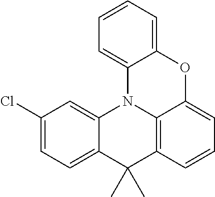 [SMILES: CC1(C)c2ccc(Cl)cc2N2c3ccccc3Oc3cccc1c32] | 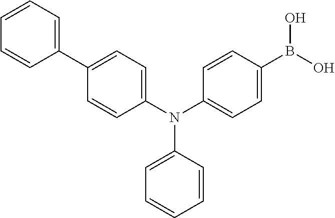 [SMILES: OB(O)c1ccc(N(c2ccccc2)c2ccc(-c3ccccc3)cc2)cc1] |
| 783 | 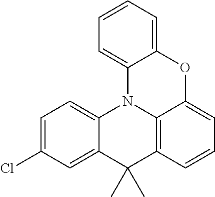 [SMILES: CC1(C)c2cc(Cl)ccc2N2c3ccccc3Oc3cccc1c32] | 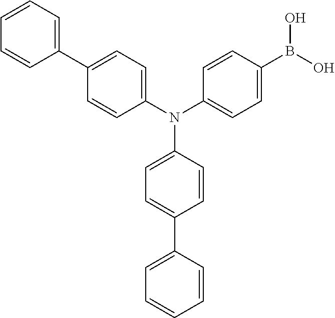 [SMILES: OB(O)c1ccc(N(c2ccc(-c3ccccc3)cc2)c2ccc(-c3ccccc3)cc2)cc1] |

| No. | Target Compound | Yield |
|---|---|---|
| 541 | 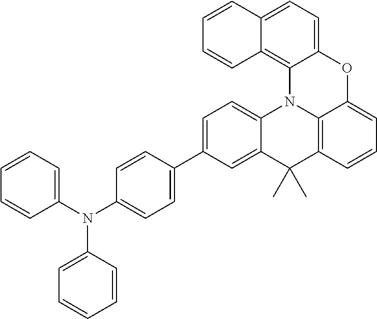 [SMILES: CC1(C)c2cc(-c3ccc(N(c4ccccc4)c4ccccc4)cc3)ccc2N2c3c(cccc31)Oc1ccc3ccccc3c12] | 78% |
| 545 | 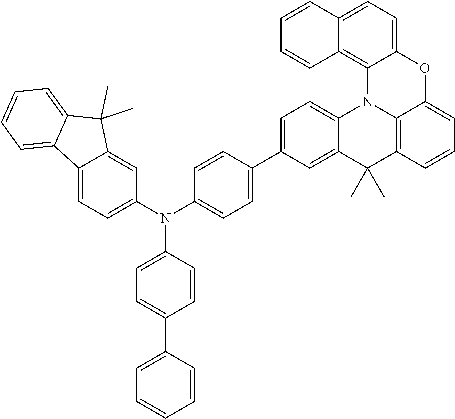 [SMILES: CC1(C)c2ccccc2-c2ccc(N(c3ccc(-c4ccccc4)cc3)c3ccc(-c4ccc5c(c4)C(C)(C)c4cccc6c4N5c4c(ccc5ccccc45)O6)cc3)cc21] | 75% |
| 603 | 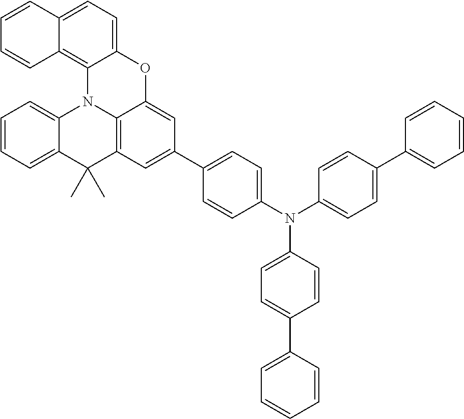 [SMILES: CC1(C)c2ccccc2N2c3c(cc(-c4ccc(N(c5ccc(-c6ccccc6)cc5)c5ccc(-c6ccccc6)cc5)cc4)cc31)Oc1ccc3ccccc3c12] | 65% |
| 622 | 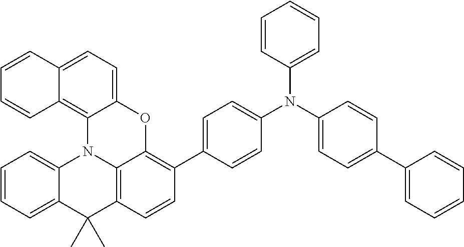 [SMILES: CC1(C)c2ccccc2N2c3c1ccc(-c1ccc(N(c4ccccc4)c4ccc(-c5ccccc5)cc4)cc1)c3Oc1ccc3ccccc3c12] | 70% |
| 651 | 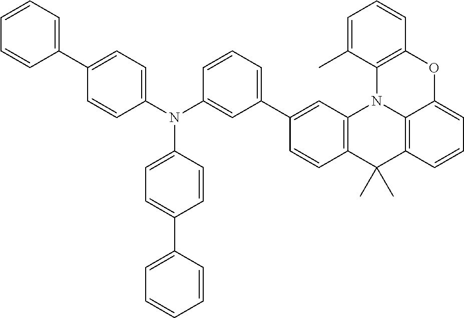 [SMILES: Cc1cccc2c1N1c3cc(-c4cccc(N(c5ccc(-c6ccccc6)cc5)c5ccc(-c6ccccc6)cc5)c4)ccc3C(C)(C)c3cccc(c31)O2] | 69% |
| 663 | 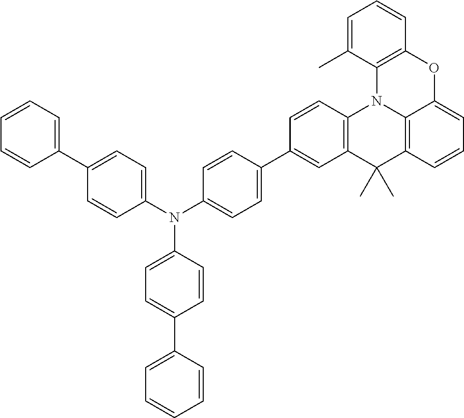 [SMILES: Cc1cccc2c1N1c3ccc(-c4ccc(N(c5ccc(-c6ccccc6)cc5)c5ccc(-c6ccccc6)cc5)cc4)cc3C(C)(C)c3cccc(c31)O2] | 73% |
| 685 | 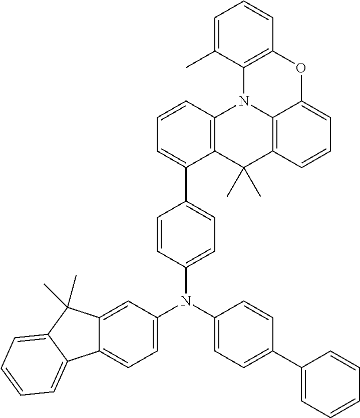 [SMILES: Cc1cccc2c1N1c3cccc(-c4ccc(N(c5ccc(-c6ccccc6)cc5)c5ccc6c(c5)C(C)(C)c5ccccc5-6)cc4)c3C(C)(C)c3cccc(c31)O2] | 77% |
| 762 | 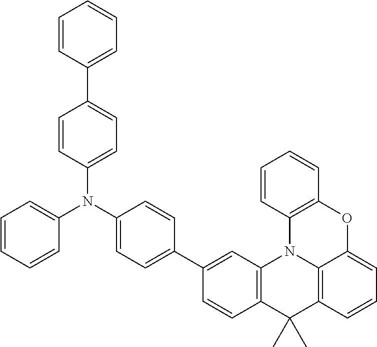 [SMILES: CC1(C)c2ccc(-c3ccc(N(c4ccccc4)c4ccc(-c5ccccc5)cc4)cc3)cc2N2c3ccccc3Oc3cccc1c32] | 74% |
| 783 | 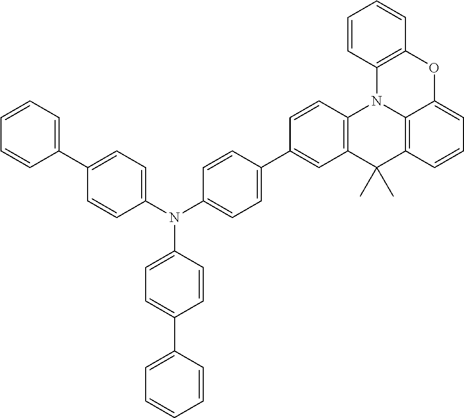 [SMILES: CC1(C)c2cc(-c3ccc(N(c4ccc(-c5ccccc5)cc4)c4ccc(-c5ccccc5)cc4)cc3)ccc2N2c3ccccc3Oc3cccc1c32] | 76% |

Compounds other than the compounds described in Preparation Example 1 to Preparation Example 3 and Table 1 to Table 3 were also prepared in the same manner as in the methods described in the preparation examples, and the synthesis results are shown in the following Table 4 and Table 5. The following Table 4 shows measurement values of ¹H NMR (CDCl₃, 300 MHz), and the following Table 5 shows measurement values of FD-mass spectrometry (FD-MS: field desorption mass spectrometry).

TABLE 4

| Compound | ¹H NMR (CDCl₃, 300 MHz) |
|---|---|
| 002 | δ = 7.98 (1H, d), 7.93 (1H, d), 7.54-7.41 (9H, m), 7.32 (1H, d), 7.20 |
| | (2H, t), 6.81-6.63 (10H, m), 5.91 (1H, d), 5.69 (1H, s), 1.72 (6H, s) |
| 003 | δ = 7.98 (1H, d), 7.93 (1H, d), 7.54-7.41 (16H, m), 7.32 (1H, d), |
| | 6.81-6.69 (9H, m), 5.91 (1H, d), 5.69 (1H, s), 1.72 (6H, s) |
| 005 | δ = 7.98 (1H, d), 7.93 (1H, d), 7.87 (1H, d), 7.62 (1H, d), 7.55-7.51 |
| | (8H, m), 7.42-7.28 (5H, m), 6.81-6.69 (8H, m), 6.58 (1H, d), 5.91 |
| | (1H, d), 5.89 (1H, s), 1.72 (12H, s) |
| 011 | δ = 7.98 (1H, d), 7.93 (1H, d), 7.87 (1H, d), 7.62 (1H, d), 7.55-7.28 |
| | (12H, m), 6.89-6.88 (2H, m), 6.81-6.69 (6H, m), 6.59-6.58 (2H, m), |
| | 5.91 (1H, d), 5.89 (1H, s), 1.72 (12H, s) |
| 030 | δ = 7.98 (1H, d), 7.93 (1H, d), 7.87 (1H, d), 7.62 (1H, d), 7.55-7.51 |
| | (5H, m), 7.42-7.28 (5H, m), 7.16 (1H, t), 7.08 (2H, d), 6.87-6.69 |
| | (7H, m), 6.58 (1H, d), 6.37 (1H, s), 6.30 (1H, d), 6.20 (1H, d), |
| | 1.72 (12H, s) |
| 031 | δ = 7.98 (1H, d), 7.93 (1H, d), 7.87 (1H, d), 7.62 (1H, d), 7.55-7.28 |
| | (12H, m), 6.89-6.88 (2H, m), 6.81-6.69 (5H, m), 6.59-6.58 (2H, m), |
| | 6.37 (1H, s), 6.30 (1H, d), 6.20 (1H, d), 1.72 (12H, s) |
| 039 | δ = 7.98 (1H, d), 7.93 (1H, d), 7.89 (1H, d), 7.66 (1H, d), 7.54-7.25 |
| | (13H, m), 7.07 (1H, t), 6.81-6.69 (6H, m), 6.39-6.37 (2H, m), 6.30 |
| | (1H, d), 6.20 (1H, d), 1.72 (6H, s) |
| 044 | δ = 7.98 (1H, d), 7.93 (1H, d), 7.87 (1H, d), 7.62 (1H, d), 7.55-7.52 |
| | (2H, m), 7.42-7.28 (4H, m), 7.20 (2H, t), 6.81-6.58 (10H, m), 5.91 |
| | (2H, d), 1.72 (12H, s) |
| 046 | δ = 7.98 (1H, d), 7.93 (1H, d), 7.87 (2H, d), 7.62 (2H, d), 7.55-7.52 |
| | (3H, m), 7.42-7.28 (6H, m), 6.81-6.69 (7H, m), 6.59 (2H, d), 5.91 |
| | (2H, d), 1.72 (18H, s) |
| 054 | δ = 7.98 (1H, d), 7.93 (1H, d), 7.87 (1H, d), 7.62 (1H, d), 7.55-7.52 |
| | (2H, m), 7.42-7.20 (12H, m), 7.11 (4H, d), 6.81-6.58 (10H, m), 5.91 |
| | (2H, d), 1.72 (6H, s) |
| 075 | δ = 7.98 (1H, d), 7.93 (1H, d), 7.87 (2H, d), 7.75 (1H, d), 7.55-7.50 |
| | (4H, m), 7.42-7.16 (11H, m), 7.05-7.02 (2H, m), 6.81-6.73 (3H, m), |
| | 6.63 (2H, d), 6.55 (2H, d), 6.46 (1H, d), 6.39 (1H, d), 5.87 (1H, |
| | d), 1.72 (6H, s) |
| 077 | δ = 8.93 (2H, d), 8.13-8.12 (3H, m), 7.98-7.82 (7H, m), 7.54-7.41 |
| | (9H, m), 7.32 (1H, d), 7.05-7.02 (3H, m), 6.81-6.69 (4H, m), 6.55 |
| | (1H, d), 6.46 (1H, d), 5.87 (1H, d), 1.72 (6H, s) |
| 083 | δ = 7.98 (1H, d), 7.93 (1H, d), 7.54-7.41 (16H, m), 7.32 (1H, d), |
| | 7.05-7.02 (2H, m), 6.81 (1H, d), 6.73-6.69 (5H, m), 6.55 (1H, d), |
| | 6.09 (1H, s), 5.90 (1H, s), 1.72 (6H, s) |
| 087 | δ = 7.98-7.74 (6H, m), 7.52-7.32 (13H, m), 7.05-7.02 (2H, m), 6.81 |
| | (1H, d), 6.73-6.69 (3H, m), 6.55 (1H, d), 6.09 (1H, s), 5.90 (1H, |
| | s), 1.72 (6H, s) |
| 098 | δ = 8.93 (2H, d), 8.12 (2H, d), 7.98-7.82 (6H, m), 7.54-7.41 (9H, m), |
| | 7.32 (1H, d), 7.05-7.02 (2H, m), 6.91 (1H, s), 6.81-6.69 (4H, m), |
| | 6.55 (1H, d), 6.09 (1H, s), 5.90 (1H, s), 1.72 (6H, s) |
| 108 | δ = 8.07-7.93 (4H, m), 7.57-7.32 (14H, m), 7.05-6.98 (3H, m), 6.81 |
| | (1H, d), 6.73-6.69 (3H, m), 6.55-6.52 (2H, m), 5.87 (1H, d), 1.72 |
| | (6H, s) |
| 112 | δ = 8.55 (1H, d), 8.42 (1H, d), 8.08-7.93 (4H, m), 7.61-7.41 (14H, |
| | m), 7.32 (1H, d), 7.05-7.02 (2H, m), 6.81-6.69 (6H, m), 6.55-6.52 |
| | (2H, m), 5.87 (1H, d), 1.72 (6H, s) |
| 119 | δ = 7.98 (1H, d), 7.93 (1H, d), 7.89 (1H, d), 7.66 (1H, d), 7.54-7.25 |
| | (13H, m), 7.07-7.02 (3H, m), 6.81 (1H, d), 6.73-6.69 (3H, m), |
| | 6.55-6.52 (2H, m), 6.39 (1H, d), 5.87 (1H, d), 1.72 (6H, s) |
| 131 | δ = 7.87 (1H, d), 7.62 (1H, d), 7.55-7.38 (8H, m), 7.28 (1H, t), |
| | 6.89-6.87 (3H, m), 6.89-6.58 (9H, m), 5.91 (1H, d), 5.69 (1H, s), |
| | 2.12 (3H, s), 1.72 (12H, s) |
| 137 | δ = 8.93 (2H, d), 8.13-8.12 (3H, m), 7.88-7.82 (5H, m), 7.54-7.41 |
| | (7H, m), 7.02 (1H, d), 6.80-6.65 (9H, m), 5.91 (1H, d), 5.69 (1H, |
| | s), 2.12 (3H, s), 1.72 (6H, s) |
| 142 | δ = 7.54-7.51 (6H, m), 7.41 (1H, t), 7.20 (2H, t), 6.87-6.63 (11H, |
| | m), 6.37 (1H, s), 6.30 (1H, d), 6.20 (1H, d), 2.12 (3H, s), 1.72 |
| | (6H, s) |
| 144 | δ = 7.87 (1H, d), 7.62 (1H, d), 7.55 (1H, d), 7.38 (1H, t), 7.28 (1H, |
| | t), 7.20 (2H, t), 6.87-6.58 (11H, m), 6.37 (1H, s), 6.30 (1H, d), |
| | 6.20 (1H, d), 2.12 (3H, s), 1.72 (12H, s) |
| 163 | δ = 7.54-7.41 (14H, m), 6.87 (1H, d), 6.77-6.65 (10H, m), 5.91 (2H, |
| | d), 2.12 (3H, s), 1.72 (6H, s) |
| 165 | δ = 7.87 (1H, d), 7.62 (1H, d), 7.55-7.51 (7H, m), 7.41-7.38 (2H, m), |
| | 7.28 (1H, t), 6.87 (1H, d), 7.77-7.65 (9H, m), 6.58 (1H, d), 5.91 |
| | (2H, d), 2.12 (3H, s), 1.72 (12H, s) |
| 170 | δ = 7.87 (1H, d), 7.62 (1H, d), 7.55-7.51 (4H, m), 7.46-7.38 (2H, m), |
| | 7.28 (1H, t), 7.16 (1H, t), 7.08 (2H, d), 6.87 (2H, m), 6.77-6.69 |
| | (8H, m), 6.58 (1H, d), 5.91 (2H, d), 2.12 (3H, s), 1.72 (12H, s) |
| 196 | δ = 8.07 (1H, d), 8.02 (1H, d), 7.88-7.74 (4H, m), 7.57-7.49 (5H, m), |
| | 7.38-7.36 (2H, m), 7.05-6.98 (3H, m), 6.87 (1H, d), 6.73-6.65 (3H, |
| | m), 6.55 (1H, d), 6.46 (1H, d), 5.87 (1H, d), 2.12 (3H, s), 1.72 |
| | (6H, s) |
| 202 | δ = 7.54-7.51 (6H, m), 7.41 (1H, t), 7.20 (2H, t), 7.05-7.02 (2H, m), |
| | 6.87-6.65 (9H, m), 6.55 (1H, d), 6.09 (1H, s), 5.90 (1H, s), 2.12 |
| | (3H, s), 1.72 (6H, s) |
| 204 | δ = 7.87 (1H, d), 7.62 (1H, d), 7.55 (1H, d), 7.38 (1H, t), 7.28 (1H, |
| | t), 7.20 (2H, t), 7.05-7.02 (2H, m), 6.87-6.58 (10H, m), 6.09 (1H, |
| | s), 5.90 (1H, s), 2.12 (3H, s), 1.72 (12H, s) |
| 205 | δ = 7.87 (1H, d), 7.62 (1H, d), 7.54-7.51 (7H, m), 7.41-7.38 (2H, m), |
| | 7.28 (1H, t), 7.05-7.02 (2H, m), 6.87 (1H, d), 6.73-6.65 (6H, m), |
| | 6.58-6.55 (2H, m), 6.09 (1H, s), 5.90 (1H, s), 2.12 (3H, s), 1.72 |
| | (12H, s) |
| 212 | δ = 8.55 (1H, d), 8.42 (1H, d), 8.08-8.04 (2H, m), 7.61-7.41 (12H, |
| | m), 7.05-7.02 (2H, m), 6.87 (1H, d), 6.73-6.65 (7H, m), 6.55 (1H, |
| | d), 6.09 (1H, s), 5.90 (1H, s), 2.12 (3H, s), 1.72 (6H, s) |
| 234 | δ = 7.87 (1H, d), 7.62 (1H, d), 7.55 (1H, d), 7.38-7.02 (16H, m), |
| | 6.87-6.52 (11H, m), 5.87 (1H, d), 2.12 (3H, s), 1.72 (6H, s) |
| 239 | δ = 7.89 (1H, d), 7.66 (1H, d), 7.54-7.51 (6H, m), 7.41-7.25 (4H, m), |
| | 7.07-7.02 (3H, m), 6.87 (1H, d), 6.73-6.65 (5H, m), 6.55-6.52 (2H, |
| | m), 6.39 (1H, d), 5.87 (1H, d), 2.12 (3H, s), 1.72 (6H, s) |
| 245 | δ = 7.87 (1H, d), 7.62 (1H, d), 7.55-7.51 (7H, m), 7.41-7.38 (2H, m), |
| | 7.28 (1H, t), 6.92-6.89 (2H, m), 6.80-6.69 (8H, m), 6.59-6.58 (2H, |
| | m), 5.91 (1H, d), 5.69 (1H, s), 1.72 (12H, s) |
| 247 | δ = 7.88-7.74 (4H, m), 7.54-7.36 (10H, m), 6.92-6.89 (2H, m), |
| | 6.80-6.69 (7H, m), 6.59 (1H, d), 5.91 (1H, d), 5.69 (1H, s), 1.72 |
| | (6H, s) |
| 253 | δ = 8.07-8.02 (2H, m), 7.87 (1H, d), 7.62-7.53 (5H, m), 7.38 (2H, t), |
| | 7.28 (1H, t), 6.98-6.89 (3H, m), 6.80-6.69 (6H, m), 6.59-6.58 (2H, |
| | m), 5.91 (1H, d), 5.69 (1H, s), 1.72 (12H, s) |
| 260 | δ = 8.45 (1H, d), 7.98 (1H, d), 7.87-7.81 (2H, m), 7.62 (1H, d), |
| | 7.55-7.50 (3H, m), 7.38 (1H, t), 7.28-7.27 (2H, m), 6.89-6.69 (9H, |
| | m), 6.59-6.58 (2H, m), 5.91 (1H, d), 5.69 (1H, s), 1.72 (12H, s) |
| 269 | δ = 7.54-7.41 (11H, m), 7.16 (1H, t), 7.08 (2H, d), 6.92-6.87 (3H, |
| | m), 6.77-6.69 (7H, m), 6.59 (1H, d), 6.37 (1H, s), 6.30 (1H, d), |
| | 6.20 (1H, d), 1.72 (6H, s) |
| 275 | δ = 7.87 (2H, d), 7.75 (1H, d), 7.55-7.50 (3H, m), 7.38-7.16 (9H, m), |
| | 6.92-6.63 (11H, m), 6.39-6.37 (2H, m), 6.30 (1H, d), 6.20 (1H, d), |
| | 1.72 (6H, s) |
| 290 | δ = 7.87 (1H, d), 7.62 (1H, d), 7.55-7.51 (4H, m), 7.41-7.38 (2H, m), |
| | 7.28 (1H, t), 7.16 (1H, t), 7.08 (2H, d), 6.92-6.87 (3H, m), |
| | 6.77-6.69 (7H, m), 6.59-6.58 (2H, m), 5.19 (2H, d), 1.72 (12H, s) |
| 302 | δ = 7.54-7.51 (6H, m), 7.41 (1H, t), 7.20 (2H, t), 7.05-7.02 (2H, m), |
| | 6.92-6.89 (2H, m), 6.81-6.55 (9H, m), 6.46 (1H, d), 5.87 (1H, d), |
| | 1.72 (6H, s) |
| 304 | δ = 7.87 (1H, d), 7.62 (1H, d), 7.55 (1H, d), 7.38 (1H, t), 7.28 (1H, |
| | t), 7.20 (2H, t), 7.05-7.02 (2H, m), 6.92-6.89 (2H, m), 6.81-6.73 |
| | (4H, m), 6.63-6.55 (5H, m), 6.46 (1H, d), 5.87 (1H, d), 1.72 (12H, s) |
| 317 | δ = 8.93 (2H, d), 8.13-8.12 (3H, m), 7.88-7.82 (5H, m), 7.54-7.41 |
| | (7H, m), 7.05-7.02 (3H, m), 6.92-6.89 (2H, m), 6.77-6.69 (4H, m), |
| | 6.59-6.55 (2H, m), 6.46 (1H, d), 5.87 (1H, d), 1.72 (6H, s) |
| 323 | δ = 7.54-7.41 (14H, m), 7.05-7.02 (2H, m), 6.92-6.89 (2H, m), |
| | 6.77-6.69 (6H, m), 6.59-6.55 (2H, m), 6.09 (1H, s), 5.90 (1H, s), |
| | 1.72 (6H, s) |
| 338 | δ = 8.93 (2H, d), 8.12 (2H, d), 7.88-7.82 (4H, m), 7.54-7.41 (7H, m), |
| | 7.05-7.02 (2H, m), 6.92-6.89 (3H, m), 6.77-6.69 (4H, m), 6.59-6.55 |
| | (2H, m), 6.09 (1H, s), 5.90 (1H, s), 1.72 (6H, s) |
| 346 | δ = 7.87 (2H, d), 7.62 (2H, d), 7.55 (2H, d), 7.38 (2H, t), 7.28 (2H, |
| | t), 7.05-7.02 (2H, m), 6.92-6.89 (2H, m), 6.77-6.73 (4H, m), |
| | 6.59-6.52 (5H, m), 5.87 (1H, d), 1.72 (18H, s) |
| 357 | δ = 8.93 (2H, d), 8.13-8.12 (3H, m), 7.88-7.82 (5H, m), 7.54-7.41 |
| | (7H, m), 7.05-7.02 (3H, m), 6.92-6.89 (2H, m), 6.77-6.69 (4H, m), |
| | 6.59-6.52 (3H, m), 5.87 (1H, d), 1.72 (6H, s) |
| 362 | δ = 7.54-7.41 (12H, m), 7.20 (2H, t), 7.05-7.02 (2H, m), 6.81-6.63 |
| | (7H, m), 6.55 (1H, d), 6.09 (1H, s), 5.90 (1H, s), 2.34 (3H, s), |
| | 1.72 (6H, s) |
| 372 | δ = 7.88-7.84 (2H, m), 7.77-7.74 (2H, m), 7.54-7.36 (15H, m), 7.26 |
| | (1H, s), 7.07 (1H, s), 6.80-6.69 (6H, m), 5.91 (1H, d), 5.69 (1H, |
| | s), 2.12 (3H, s), 1.72 (6H, s) |
| 383 | δ = 7.52-7.41 (11H, m), 7.33 (1H, s), 7.20 (2H, t), 7.08 (1H, s), |
| | 6.89-6.81 (4H, m), 6.70-6.59 (5H, m), 6.37 (1H, s), 6.30 (1H, d), |
| | 6.20 (1H, d), 2.12 (3H, s), 1.72 (6H, s) |
| 405 | δ = 7.54-7.41 (19H, m), 7.11 (1H, d), 6.81-6.69 (9H, m), 6.45 (1H, |
| | d), 5.83 (1H, d), 2.12 (3H, s), 1.72 (6H, s) |
| 412 | δ = 7.88-7.84 (2H, m), 7.77-7.74 (2H, m), 7.54-7.36 (15H, m), 7.26 |
| | (1H, t), 7.07 (1H, d), 6.87 (1H, d), 6.70-6.65 (4H, m), 6.51-6.46 |
| | (2H, m), 5.87 (1H, d), 2.12 (3H, s), 1.72 (6H, s) |
| 426 | δ = 7.98 (1H, d), 7.92 (1H, d), 7.54-7.41 (18H, m), 7.32 (1H, d), |
| | 7.16-7.08 (4H, m), 6.87-6.81 (3H, m), 6.73-6.69 (4H, m), 6.52 (1H, |
| | d), 5.87 (1H, d), 1.72 (6H, s) |
| 449 | δ = 7.87 (1H, d), 7.62 (1H, d), 7.55-7.51 (11H, m), 7.41-7.38 (3H, |
| | m), 7.28-7.26 (3H, m), 6.77-6.65 (7H, m), 6.58 (1H, d), 6.37 (1H, |
| | s), 6.30 (1H, d), 6.20 (1H, d), 1.72 (12H, s) |
| 471 | δ = 7.87 (1H, d), 7.62 (1H, d), 7.55-7.51 (8H, m), 7.41-7.28 (5H, m), |
| | 7.08-7.02 (5H, m), 6.87 (1H, t), 6.75-6.55 (6H, m), 5.95 (1H, d), |
| | 5.73 (1H, s), 1.72 (12H, s) |
| 479 | δ = 7.88-7.84 (3H, m), 7.77-7.74 (2H, m), 7.62 (1H, d), 7.55-7.41 |
| | (8H, m), 7.38-7.26 (4H, m), 7.07 (1H, d), 6.77-6.69 (4H, m), 6.58 |
| | (1H, d), 6.51 (1H, d), 6.34 (1H, d), 6.10 (2H, m), 1.72 (12H, s) |
| 505 | δ = 8.55 (1H, d), 8.42 (1H, d), 8.08-8.04 (2H, m), 7.61-7.41 (17H, |
| | m), 7.26 (1H, t), 7.07 (1H, d), 6.77-6.67 (8H, m), 6.51 (1H, d), |
| | 5.95 (2H, d), 1.72 (6H, s) |
| 519 | δ = 8.93 (2H, d), 8.12 (2H, d), 7.93-7.82 (5H, m), 7.52-7.44 (6H, m), |
| | 7.33 (1H, s), 7.20 (2H, t), 7.08-7.02 (3H, m), 6.89-6.55 (9H, m), |
| | 5.95 (2H, d), 1.72 (6H, s) |
| 535 | δ = 7.98 (1H, d), 7.93 (1H, d), 7.54-7.41 (15H, m), 7.32 (1H, d), |
| | 7.16-7.08 (4H, m), 6.87-6.69 (12H, m), 1.72 (6H, s) |
| 541 | δ = 7.98 (1H, d), 7.93 (1H, d), 7.61 (1H, s), 7.54-7.52 (3H, m), |
| | 7.42-7.32 (3H, m), 7.20 (4H, t), 6.81-6.63 (13H, m), 1.72 (6H, s) |
| 545 | δ = 7.98 (1H, d), 7.93 (1H, d), 7.87 (1H, d), 7.62-7.61 (2H m), |
| | 7.55-7.51 (10H, m), 7.42-7.28 (6H, m), 6.81-6.69 (9H, m), 6.61-6.58 |
| | (2H, m), 1.72 (12H, s) |
| 603 | δ = 7.98 (1H, d), 7.93 (1H, d), 7.54-7.41 (18H, m), 7.33-7.32 (2H, |
| | m), 7.08-7.02 (3H, m), 6.81 (1H, d), 6.73-6.69 (7H, m), 6.55 (1H, |
| | d), 1.72 (6H, s) |
| 622 | δ = 7.98 (1H, d), 7.93 (1H, d), 7.54-7.41 (11H, m), 7.32 (1H, d), |
| | 7.20 (2H, t), 7.05-7.02 (3H, m), 6.83-6.63 (10H, m), 6.55 (1H, d), |
| | 1.72 (6H, s) |
| 651 | δ = 7.54-7.44 (15H, m), 7.11 (1H, d), 6.89-6.87 (3H, m), 6.77-6.59 |
| | (12H, m), 2.12 (3H, s), 1.72 (6H, s) |
| 663 | δ = 7.61 (1H, s), 7.54-7.51 (14H, m), 7.41-7.36 (3H, m), 6.87 (1H, |
| | d), 6.77-6.65 (12H, m), 2.12 (3H, s), 1.72 (6H, s) |
| 685 | δ = 7.87 (1H, d), 7.62 (1H, d), 7.55-7.51 (9H, m), 7.41-7.38 (2H, m), |
| | 7.28-7.26 (2H, m), 7.07 (1H, d), 6.87 (1H, d), 6.77-6.65 (10H, m), |
| | 6.58 (1H, d), 6.51 (1H, d), 2.12 (3H, s), 1.72 (6H, s) |
| 762 | δ = 7.54-7.51 (8H, m), 7.41 (1H, t), 7.20 (2H, t), 7.11 (1H, d), |
| | 6.92-6.89 (2H, m), 6.81-6.59 (14H, m), 1.72 (6H, s) |
| 783 | δ = 7.61 (1H, s), 7.54-7.51 (14H, m), 7.41-7.36 (3H, m), 6.92-6.89 |
| | (2H, m), 6.77-6.69 (10H, m), 6.61-6.59 (2H, m), 1.72 (6H, s) |

TABLE 5

| Compound | FD-MS | Compound | FD-MS |
|---|---|---|---|
| 002 | m/z = 592.73 (C₄₃H₃₂N₂O = 592.25) | 003 | m/z = 668.82 (C₄₉H₃₆N₂O = 668.28) |
| 005 | m/z = 708.89 (C₅₂H₄₀N₂O = 708.31) | 011 | m/z = 708.89 (C₅₂H₄₀N₂O = 708.31) |
| 030 | m/z = 708.89 (C₅₂H₄₀N₂O = 708.31) | 031 | m/z = 708.89 (C₅₂H₄₀N₂O = 708.31) |
| 039 | m/z = 682.81 (C₄₉H₃₉N₂O₂ = 682.26) | 044 | m/z = 632.79 (C₄₆H₃₆N₂O = 632.28) |
| 046 | m/z = 748.95 (C₅₅H₄₄N₂O = 748.35) | 054 | m/z = 756.93 (C₅₆H₄₀N₂O = 756.31) |
| 075 | m/z = 754.91 (C₅₆H₃₈N₂O = 754.30) | 077 | m/z = 742.90 (C₅₅H₃₈N₂O = 742.30) |
| 083 | m/z = 668.82 (C₄₉H₃₆N₂O = 668.28) | 087 | m/z = 642.79 (C₄₇H₃₄N₂O = 642.27) |
| 098 | m/z = 692.84 (C₅₁H₃₆N₂O = 692.28) | 108 | m/z = 642.79 (C₄₇H₃₄N₂O = 642.27) |
| 112 | m/z = 718.88 (C₅₃H₃₈N₂O = 718.30) | 119 | m/z = 682.81 (C₄₉H₃₉N₂O₂ = 682.26) |
| 131 | m/z = 672.85 (C₄₉H₄₀N₂O = 672.31) | 137 | m/z = 706.87 (C₅₂H₃₈N₂O = 706.30) |
| 142 | m/z = 556.69 (C₄₀H₃₂N₂O = 556.25) | 144 | m/z = 596.76 (C₄₃H₃₆N₂O = 596.28) |
| 163 | m/z = 632.79 (C₄₆H₃₆N₂O = 632.28) | 165 | m/z = 672.85 (C₄₉H₄₀N₂O = 672.31) |
| 170 | m/z = 672.85 (C₄₉H₄₀N₂O = 672.31) | 196 | m/z = 580.72 (C₄₂H₃₂N₂O = 580.25) |
| 202 | m/z = 556.69 (C₄₀H₃₂N₂O = 556.25) | 204 | m/z = 596.76 (C₄₃H₃₆N₂O = 596.28) |
| 205 | m/z = 672.85 (C₄₉H₄₀N₂O = 672.31) | 212 | m/z = 682.85 (C₅₀H₃₈N₂O = 682.30) |
| 234 | m/z = 720.90 (C₅₃H₄₀N₂O = 720.31) | 239 | m/z = 646.77 (C₄₆H₃₄N₂O₂ = 646.26) |
| 245 | m/z = 658.83 (C₄₈H₃₈N₂O = 658.30) | 247 | m/z = 592.73 (C₄₃H₃₂N₂O = 592.25) |
| 253 | m/z = 632.79 (C₄₆H₃₆N₂O = 632.28) | 260 | m/z = 688.88 (C₄₈H₃₆N₂OS = 688.25) |
| 269 | m/z = 618.76 (C₄₅H₃₄N₂O = 618.27) | 275 | m/z = 704.86 (C₅₂H₃₆N₂O = 704.28) |
| 290 | m/z = 658.83 (C₄₈H₃₈N₂O = 658.30) | 302 | m/z = 542.67 (C₃₉H₃₀N₂O = 542.24) |
| 304 | m/z = 582.73 (C₄₂H₃₄N₂O = 582.27) | 317 | m/z = 692.84 (C₅₁H₃₆N₂O = 692.28) |
| 323 | m/z = 618.76 (C₄₅H₃₄N₂O = 618.27) | 338 | m/z = 642.79 (C₄₇H₃₄N₂O = 642.27) |
| 346 | m/z = 658.83 (C₄₈H₃₈N₂O = 658.30) | 357 | m/z = 692.84 (C₅₁H₃₆N₂O = 692.28) |
| 362 | m/z = 632.79 (C₄₆H₃₆N₂O = 632.28) | 372 | m/z = 682.85 (C₅₀H₃₈N₂O = 682.30) |
| 383 | m/z = 682.85 (C₅₀H₃₈N₂O = 682.30) | 405 | m/z = 708.89 (C₅₂H₄₀N₂O = 708.31) |
| 412 | m/z = 682.85 (C₅₀H₃₈N₂O = 682.30) | 426 | m/z = 744.92 (C₅₅H₄₀N₂O = 744.31) |
| 449 | m/z = 734.92 (C₅₄H₄₂N₂O = 734.33) | 471 | m/z = 734.92 (C₅₄H₄₂N₂O = 734.33) |
| 479 | m/z = 708.89 (C₅₂H₄₀N₂O = 708.31) | 505 | m/z = 744.92 (C₅₅H₄₀N₂O = 744.31) |
| 519 | m/z = 718.88 (C₅₃H₃₈N₂O = 718.30) | 535 | m/z = 744.92 (C₅₅H₄₀N₂O = 744.31) |
| 541 | m/z = 592.73 (C₄₃H₃₂N₂O = 592.25) | 545 | m/z = 784.98 (C₅₈H₄₄N₂O = 784.35) |
| 603 | m/z = 744.92 (C₅₅H₄₀N₂O = 744.31) | 622 | m/z = 668.82 (C₄₉H₃₆N₂O = 668.28) |
| 651 | m/z = 708.89 (C₅₂H₄₀N₂O = 708.31) | 663 | m/z = 708.89 (C₅₂H₄₀N₂O = 708.31) |
| 685 | m/z = 748.95 (C₅₅H₄₄N₂O = 748.35) | 762 | m/z = 618.76 (C₄₅H₃₄N₂O = 618.27) |
| 783 | m/z = 694.86 (C₅₁H₃₈N₂O = 694.30) | | |

### Experimental Example 1

(1) Manufacture of Organic Light Emitting Device

A glass substrate on which ITO (indium tin oxide) was coated as a thin film to a thickness of 1, 500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function increase and residual film removal, the substrate was transferred to a thermal deposition apparatus for organic deposition.

Subsequently, the chamber was evacuated until the degree of vacuum therein reached 10⁻⁶ torr, and then 2-TNATA was evaporated by applying a current to the cell to deposit a hole injection layer on the ITO substrate to a thickness of 600 Å. To another cell in the vacuum deposition apparatus, a compound described in the following Table 6 was introduced, and evaporated by applying a current to the cell to deposit a hole transfer layer having a thickness of 300 Å on the hole injection layer.

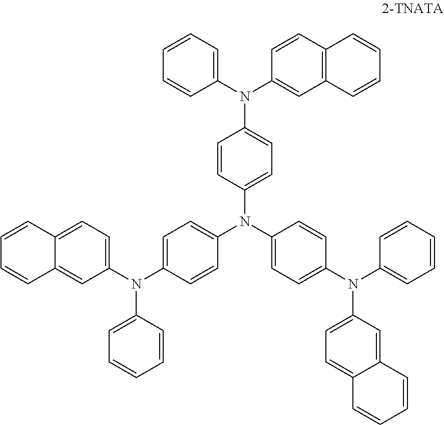 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

A light emitting layer was thermal vacuum deposited thereon as follows. As the light emitting layer, a compound of 9-[4-(4,6-diphenyl-1,3,5-triazin-2-yl)phenyl]-9′-phenyl-3,3′-bi-9H-carbazole was deposited to a thickness of 400 Å as a host, and a green phosphorescent dopant [Ir(ppy)₃] was deposited by being doped to the host by 7% with respect to the weight of the host material. After that, BCP (bathocuproine) was deposited to a thickness of 60 Å as a hole blocking layer, and Alq₃ was deposited to a thickness of 200 Å thereon as an electron transfer layer.

Lastly, an electron injection layer was formed on the electron transfer layer by depositing lithium fluoride (LiF) to a thickness of 10 Å, and then a negative electrode was formed on the electron injection layer by depositing an aluminum (Al) negative electrode to a thickness of 1,200 Å, and as a result, an organic electroluminescent device was manufactured.

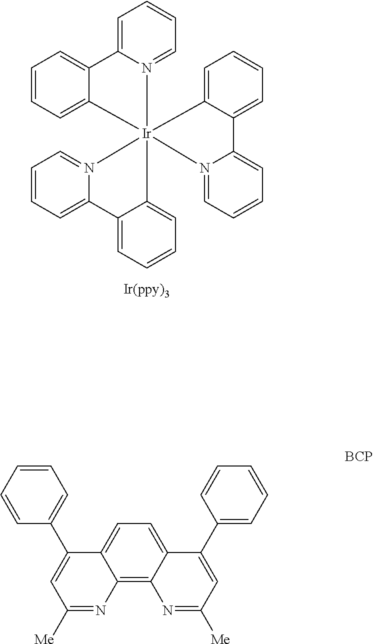

Meanwhile, a the organic compounds require to manufacture the OLED were vacuum sublimation purified under 10⁻⁸ torr to 10⁻⁶ torr for each material to be used in the OLED manufacture.

Herein, the comparative compounds used as the hole transfer layer of the following comparative examples are as follows.

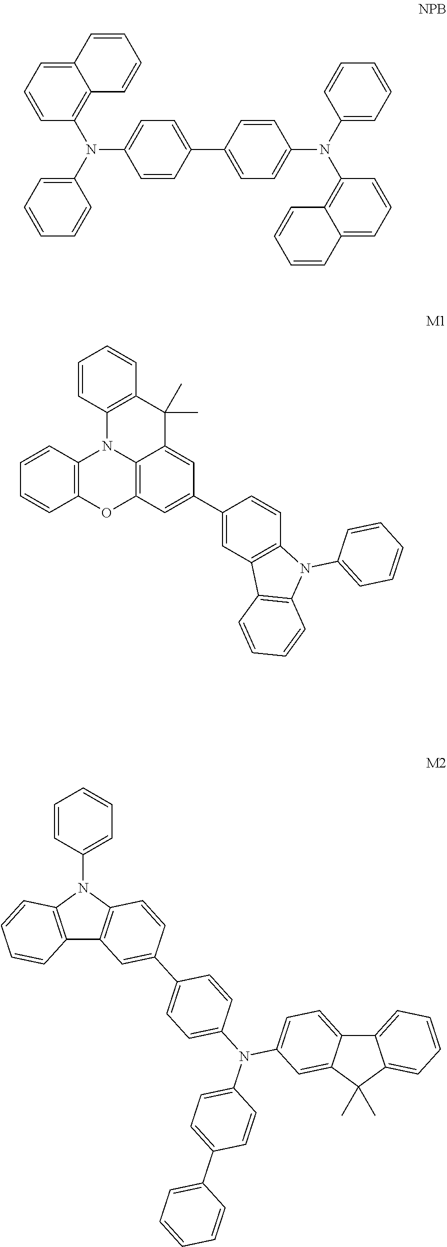 [SMILES: c1ccc(N(c2ccc(-c3ccc(N(c4ccccc4)c4cccc5ccccc45)cc3)cc2)c2cccc3ccccc23)cc1 ; CC1(C)c2ccccc2N2c3ccccc3Oc3cc(-c4ccc5c(c4)c4ccccc4n5-c4ccccc4)cc1c32 ; CC1(C)c2ccccc2-c2ccc(N(c3ccc(-c4ccccc4)cc3)c3ccc(-c4ccc5c(c4)c4ccccc4n5-c4ccccc4)cc3)cc21]

(2) Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, T₉₀ was measured when standard luminance was 6,000 cd/in² through a lifetime measurement system (M6000) manufactured by McScience Inc.

Properties of the organic electroluminescent devices of the present disclosure are as shown in the following Table 6.

TABLE 6

| | | Driving | Light Emission | Lifetime |
|---|---|---|---|---|
| | Compound | Voltage (V) | Efficiency (cd/A) | (T₉₀) |
| Example 1 | 002 | 4.15 | 121.42 | 145 |
| Example 2 | 003 | 4.21 | 118.53 | 133 |
| Example 3 | 005 | 4.06 | 123.94 | 142 |
| Example 4 | 011 | 4.09 | 125.22 | 151 |
| Example 5 | 030 | 4.14 | 115.11 | 144 |
| Example 6 | 031 | 4.09 | 117.56 | 136 |
| Example 7 | 039 | 4.05 | 118.32 | 129 |
| Example 8 | 044 | 4.10 | 115.73 | 150 |
| Example 9 | 046 | 4.25 | 120.12 | 139 |
| Example 10 | 054 | 4.05 | 116.42 | 145 |
| Example 11 | 075 | 4.01 | 118.95 | 142 |
| Example 12 | 077 | 4.33 | 120.35 | 144 |
| Example 13 | 083 | 3.99 | 118.59 | 123 |
| Example 14 | 087 | 3.96 | 113.32 | 142 |
| Example 15 | 098 | 3.90 | 120.17 | 138 |
| Example 16 | 108 | 4.07 | 120.81 | 130 |
| Example 17 | 112 | 4.10 | 121.43 | 123 |
| Example 18 | 119 | 4.01 | 120.10 | 147 |
| Example 19 | 131 | 3.96 | 118.77 | 144 |
| Example 20 | 137 | 4.03 | 119.98 | 142 |
| Example 21 | 142 | 4.11 | 116.99 | 135 |
| Example 22 | 144 | 4.04 | 119.86 | 149 |
| Example 23 | 163 | 3.88 | 121.05 | 142 |
| Example 24 | 165 | 4.07 | 123.79 | 141 |
| Example 25 | 170 | 4.15 | 119.51 | 135 |
| Example 26 | 196 | 4.13 | 120.25 | 151 |
| Example 27 | 202 | 4.05 | 121.11 | 136 |
| Example 28 | 204 | 3.96 | 120.76 | 124 |
| Example 29 | 205 | 3.96 | 119.97 | 131 |
| Example 30 | 212 | 4.11 | 120.65 | 133 |
| Example 31 | 234 | 3.87 | 120.07 | 147 |
| Example 32 | 239 | 4.11 | 121.09 | 148 |
| Example 33 | 245 | 4.07 | 121.54 | 146 |
| Example 34 | 247 | 4.10 | 117.86 | 137 |
| Example 35 | 253 | 3.89 | 115.77 | 136 |
| Example 36 | 260 | 4.18 | 120.14 | 149 |
| Example 37 | 269 | 3.90 | 121.54 | 136 |
| Example 38 | 275 | 4.06 | 120.76 | 126 |
| Example 39 | 290 | 4.02 | 116.30 | 128 |
| Example 40 | 302 | 3.83 | 117.38 | 147 |
| Example 41 | 304 | 4.18 | 114.31 | 140 |
| Example 42 | 317 | 4.17 | 120.72 | 138 |
| Example 43 | 323 | 3.93 | 119.45 | 142 |
| Example 44 | 338 | 3.92 | 120.70 | 140 |
| Example 45 | 346 | 4.11 | 120.09 | 134 |
| Example 46 | 357 | 3.96 | 121.39 | 137 |
| Example 47 | 362 | 3.84 | 119.74 | 132 |
| Example 48 | 372 | 4.19 | 117.37 | 141 |
| Example 49 | 383 | 4.22 | 120.33 | 147 |
| Example 50 | 405 | 3.98 | 115.34 | 136 |
| Example 51 | 412 | 4.13 | 120.17 | 150 |
| Example 52 | 426 | 4.16 | 121.41 | 149 |
| Example 53 | 449 | 4.07 | 120.11 | 135 |
| Example 54 | 471 | 3.95 | 120.84 | 132 |
| Example 55 | 479 | 4.12 | 120.45 | 146 |
| Example 56 | 505 | 4.15 | 120.19 | 129 |
| Example 57 | 519 | 4.01 | 112.71 | 125 |
| Example 58 | 535 | 3.98 | 116.48 | 132 |
| Example 59 | 541 | 4.07 | 118.96 | 144 |
| Example 60 | 545 | 4.16 | 121.86 | 131 |
| Example 61 | 603 | 4.13 | 121.25 | 125 |
| Example 62 | 622 | 4.04 | 120.79 | 126 |
| Example 63 | 651 | 3.95 | 119.81 | 130 |
| Example 64 | 663 | 3.97 | 120.06 | 144 |
| Example 65 | 685 | 4.13 | 112.51 | 132 |
| Example 66 | 762 | 3.95 | 118.76 | 151 |
| Example 67 | 783 | 4.17 | 119.24 | 135 |
| Comparative | NPB | 4.55 | 101.01 | 113 |
| Example 1 | | | | |
| Comparative | M1 | 5.49 | 87.41 | 112 |
| Example 2 | | | | |
| Comparative | M2 | 4.46 | 105.77 | 122 |
| Example 3 | | | | |

It was identified that the organic light emitting devices of Examples 1 to 67 according to one embodiment of the present disclosure had lower driving voltage, and superior efficiency and lifetime compared to the organic light emitting devices of Comparative Examples 1 to 3.

### Experimental Example 2

(1) Manufacture of Organic Light Emitting Device

A transparent electrode ITO thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used. Next, the ITO substrate was installed in a substrate folder of a vacuum deposition apparatus, and the following 4,4′,4″-tris(N,N-(2-naphthyl)-phenylamino)triphenyl amine (2-TNATA) was introduced to a cell in the vacuum deposition apparatus.

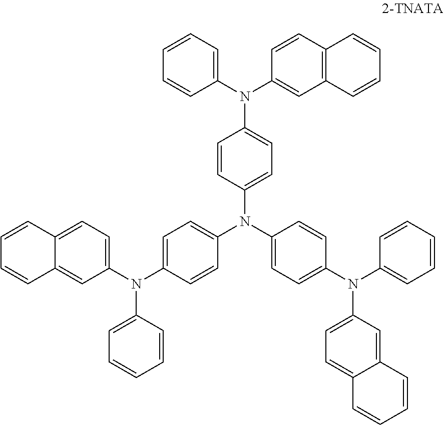 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate. To another cell in the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 150 Å on the hole injection layer. After that, an electron blocking layer having a thickness of 50 Å was formed on the hole transfer layer using a compound described in the following Table 7.

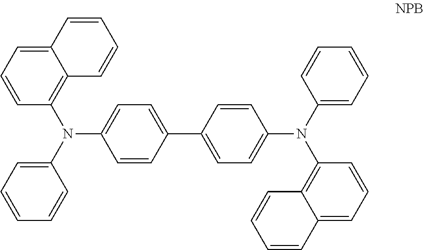 [SMILES: c1ccc(N(c2ccc(-c3ccc(N(c4ccccc4)c4cccc5ccccc45)cc3)cc2)c2cccc3ccccc23)cc1]

After forming the hole injection layer, the hole transfer layer and the electron blocking layer as above, a blue light emitting material having a structure as below was deposited thereon as a light emitting layer. Specifically, in one cell in the vacuum deposition apparatus, H1, a blue light emitting host material, was vacuum deposited to a thickness of 200 Å, and D1, a blue light emitting dopant material, was vacuum deposited thereon by 5% with respect to the weight of the host material.

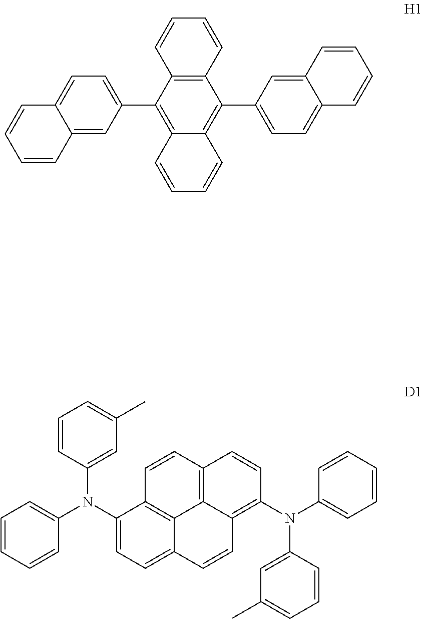

Subsequently, a compound of the following Structural Formula E1 was deposited to a thickness of 300 Å as an electron transfer layer.

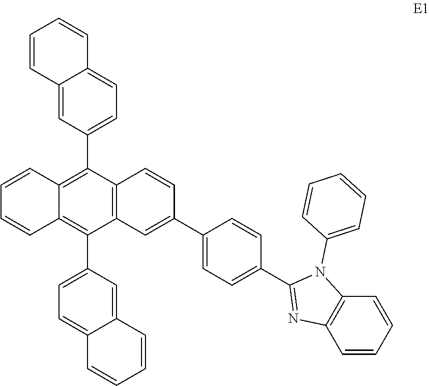 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

As an electron injection layer, lithium fluoride (LiF) was deposited to a thickness of 10 Å, and an Al negative electrode was deposited to a thickness of 1,000 Å, and as a result, an OLED was manufactured.

Meanwhile, all the organic compounds required to manufacture the OLED were vacuum sublimation purified under 10⁻⁸ torr to 10⁻⁶ torr by each material to be used in the OLED manufacture.

Herein, the comparative compounds used as the electron blocking layer of the following comparative examples are as follows.

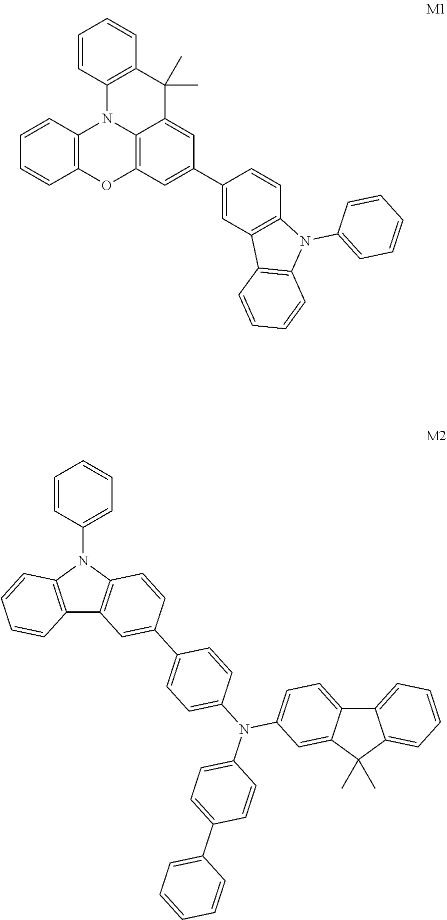 [SMILES: CC1(C)c2ccccc2N2c3ccccc3Oc3cc(-c4ccc5c(c4)c4ccccc4n5-c4ccccc4)cc1c32 ; CC1(C)c2ccccc2-c2ccc(N(c3ccc(-c4ccccc4)cc3)c3ccc(-c4ccc5c(c4)c4ccccc4n5-c4ccccc4)cc3)cc21]

(2) Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, T₉₅ was measured when standard luminance was 6,000 cd/m² through a lifetime measurement system (M6000) manufactured by McScience Inc.

Properties of the organic electroluminescent devices of the present disclosure are as shown in the following Table 7.

TABLE 7

| | | Driving | Light Emission | Lifetime |
|---|---|---|---|---|
| | Compound | Voltage (V) | Efficiency (cd/A) | (T₉₅) |
| Example 68 | 039 | 5.44 | 7.21 | 55 |
| Example 69 | 054 | 5.38 | 6.99 | 60 |
| Example 70 | 119 | 5.35 | 6.81 | 49 |
| Example 71 | 131 | 5.31 | 7.13 | 57 |
| Example 72 | 137 | 5.28 | 6.92 | 53 |
| Example 73 | 163 | 5.26 | 6.70 | 60 |
| Example 74 | 234 | 5.29 | 7.01 | 51 |
| Example 75 | 239 | 5.31 | 6.86 | 55 |
| Example 76 | 247 | 5.43 | 6.92 | 59 |
| Example 77 | 269 | 5.33 | 6.97 | 61 |
| Example 78 | 290 | 5.35 | 6.82 | 52 |
| Example 79 | 304 | 5.40 | 6.95 | 57 |
| Example 80 | 317 | 5.31 | 7.21 | 60 |
| Example 81 | 323 | 5.36 | 6.94 | 53 |
| Example 82 | 338 | 5.41 | 7.16 | 56 |
| Example 83 | 357 | 5.33 | 7.18 | 59 |
| Example 84 | 362 | 5.30 | 6.78 | 55 |
| Example 85 | 383 | 5.37 | 6.92 | 53 |
| Example 86 | 405 | 5.25 | 7.21 | 53 |
| Example 87 | 426 | 5.38 | 6.87 | 59 |
| Example 88 | 479 | 5.36 | 6.93 | 54 |
| Example 89 | 535 | 5.35 | 6.83 | 55 |
| Example 90 | 541 | 5.29 | 7.01 | 60 |
| Example 91 | 545 | 5.38 | 7.10 | 61 |
| Example 92 | 603 | 5.36 | 6.92 | 54 |
| Example 93 | 622 | 5.30 | 7.12 | 55 |
| Example 94 | 783 | 5.28 | 7.03 | 50 |
| Comparative | M1 | 5.97 | 5.86 | 37 |
| Example 4 | | | | |
| Comparative | M2 | 5.61 | 6.59 | 44 |
| Example 5 | | | | |

It was identified that the organic light emitting devices of Examples 68 to 94 according to one embodiment of the present disclosure had lower driving voltage, and superior efficiency and lifetime compared to the organic light emitting devices of Comparative Examples 4 and 5.

### Experimental Example 3

(1) Manufacture of Organic Light Emitting Device

A glass substrate on which ITO (indium tin oxide) was coated as a thin film to a thickness of 1, 500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function increase and residual film removal, the substrate was transferred to a thermal deposition apparatus for organic deposition.

Subsequently, the chamber was evacuated until the degree of vacuum therein reached 10⁻⁶ torr, and then 2-TNATA was evaporated by applying a current to the cell to deposit a hole injection layer on the ITO substrate to a thickness of 600 Å. The following N,N′-bis(α-naphthyl)-N,N′-diphenyl-4,4′-diamine (NPB) was introduced and evaporated by applying a current to the cell to deposit a hole transfer layer having a thickness of 300 Å on the hole injection layer.

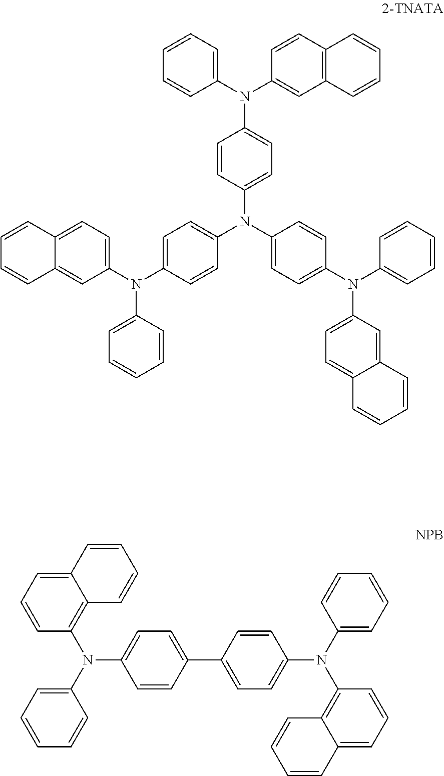 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1]

A light emitting layer was thermal vacuum deposited thereon as follows. The light emitting layer was deposited to a thickness of 500 Å using, as a host, a compound described in the following Table 8 as a single host or using an n-host (n-type host) having a favorable electron transfer ability as a first host and a p-host (p-type host) having a favorable hole transfer ability as a second host in a manner of depositing the two host compounds in one source of supply, and either doping a red phosphorescent dopant [(piq)₂(Ir) (acac)] to the host by 3% with respect to the weight of the host material or doping a green phosphorescent dopant [Ir(ppy)₃] to the host by 7% with respect to the weight of the host material.

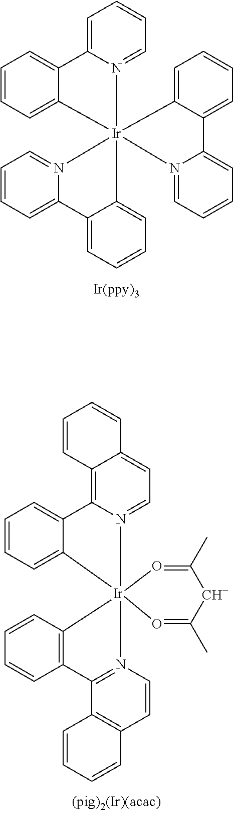

Herein, when using the two hosts, the compound used as the n-host is as follows.

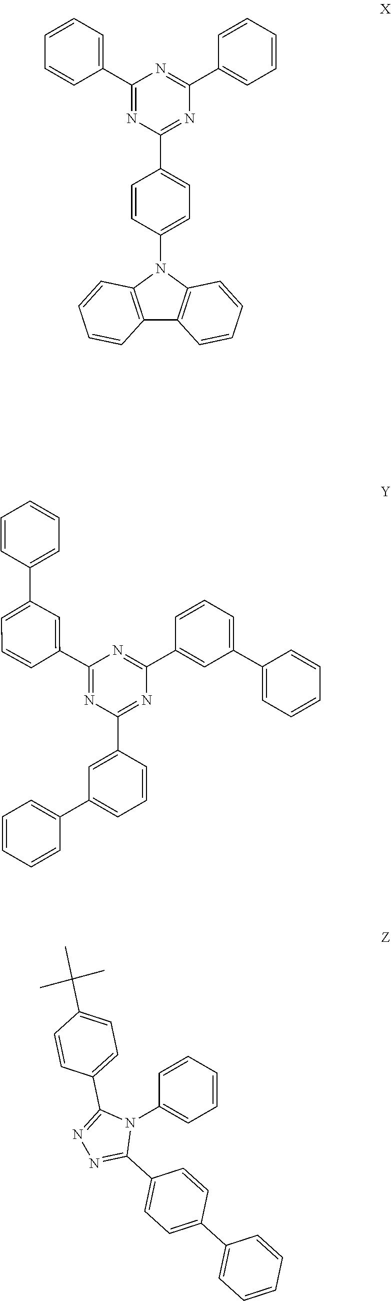 [SMILES: c1ccc(-c2nc(-c3ccccc3)nc(-c3ccc(-n4c5ccccc5c5ccccc54)cc3)n2)cc1 ; c1ccc(-c2cccc(-c3nc(-c4cccc(-c5ccccc5)c4)nc(-c4cccc(-c5ccccc5)c4)n3)c2)cc1 ; CC(C)(C)c1ccc(-c2nnc(-c3ccc(-c4ccccc4)cc3)n2-c2ccccc2)cc1]

After that, BCP (bathocuproine) was deposited to a thickness of 60 Å as a hole blocking layer, and Alq₃ was deposited to a thickness of 200 Å thereon as an electron transfer layer. Lastly, an electron injection layer was formed on the electron transfer layer by depositing lithium fluoride (LiF) to a thickness of 10 Å, and then a negative electrode was formed on the electron injection layer by depositing an aluminum (Al) negative electrode to a thickness of 1,200 Å, and as a result, an organic electroluminescent device was manufactured.

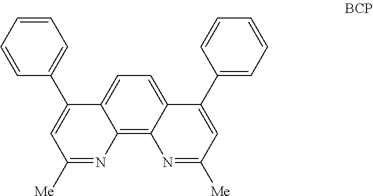 [SMILES: Cc1cc(-c2ccccc2)c2ccc3c(-c4ccccc4)cc(C)nc3c2n1]

Meanwhile, all the organic compounds required to manufacture the OLED were vacuum sublimation purified under 10⁻⁸ torr to 10⁻⁶ torr by each material to be used in the OLED manufacture.

Herein, the comparative compounds used as the host of the following comparative examples are as follows.

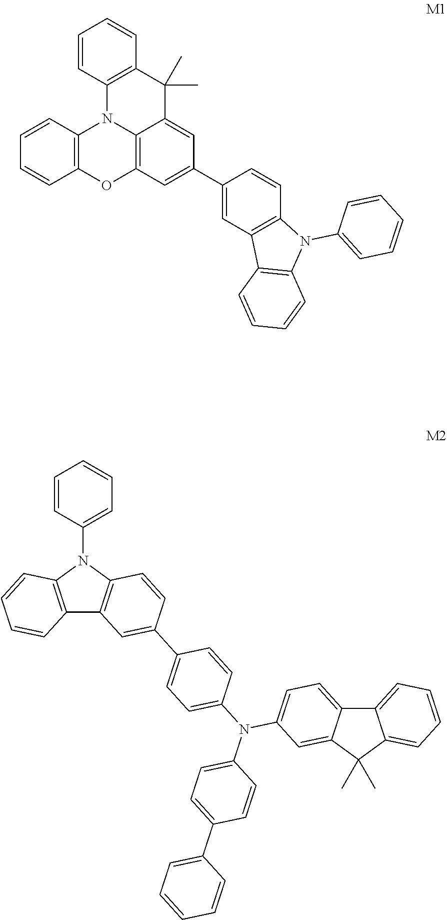 [SMILES: CC1(C)c2ccccc2N2c3ccccc3Oc3cc(-c4ccc5c(c4)c4ccccc4n5-c4ccccc4)cc1c32 ; CC1(C)c2ccccc2-c2ccc(N(c3ccc(-c4ccccc4)cc3)c3ccc(-c4ccc5c(c4)c4ccccc4n5-c4ccccc4)cc3)cc21]

(2) Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, T₉₅ was measured when standard luminance was 6,000 cd/m² through a lifetime measurement system (M6000) manufactured by McScience Inc.

Properties of the organic electroluminescent devices of the present disclosure are as shown in the following Table 8.

TABLE 8

| | | | | Light | | |
|---|---|---|---|---|---|---|
| | | | Driving | Emission | Light | Life- |
| | First | Second | Voltage | Efficiency | Emitting | time |
| | Host | Host | (V) | (cd/A) | Color | (T₉₅) |
| Example 95 | 083 | | 4.23 | 25.3 | Red | 68 |
| Example 96 | | | 4.22 | 67.8 | Green | 78 |
| Example 97 | 137 | | 4.19 | 24.7 | Red | 63 |
| Example 98 | | | 4.26 | 72.2 | Green | 80 |
| Example 99 | 239 | | 4.21 | 25.7 | Red | 66 |
| Example 100 | | | 4.19 | 70.3 | Green | 82 |
| Example 101 | 535 | | 4.18 | 25.3 | Red | 65 |
| Example 102 | | | 4.27 | 70.6 | Green | 79 |
| Example 103 | 783 | | 4.06 | 24.9 | Red | 70 |
| Example 104 | | | 4.16 | 69.6 | Green | 81 |
| Example 105 | X | 103 | 3.83 | 45.5 | Red | 133 |
| Example 106 | | 116 | 3.95 | 43.0 | Red | 152 |
| Example 107 | | 119 | 3.92 | 44.5 | Red | 147 |
| Example 108 | | 319 | 3.87 | 41.6 | Red | 136 |
| Example 109 | | 333 | 3.88 | 42.8 | Red | 148 |
| Example 110 | Y | 103 | 3.83 | 112.3 | Green | 168 |
| Example 111 | | 116 | 3.94 | 107.1 | Green | 162 |
| Example 112 | | 119 | 3.83 | 111.8 | Green | 161 |
| Example 113 | | 319 | 3.88 | 113.7 | Green | 170 |
| Example 114 | | 333 | 4.01 | 109.8 | Green | 163 |
| Example 115 | Z | 103 | 3.91 | 43.5 | Red | 139 |
| Example 116 | | 116 | 3.97 | 41.2 | Red | 142 |
| Example 117 | | 119 | 3.90 | 41.7 | Red | 140 |
| Example 118 | | 319 | 3.99 | 43.8 | Red | 145 |
| Example 119 | | 333 | 3.84 | 42.3 | Red | 151 |
| Comparative | M1 | | 4.46 | 21.1 | Red | 52 |
| Example 6 | | | | | | |
| Comparative | X | M1 | 4.22 | 34.3 | Red | 111 |
| Example 7 | | | | | | |
| Comparative | M2 | | 4.57 | 63.2 | Green | 65 |
| Example 8 | | | | | | |
| Comparative | Y | M2 | 4.32 | 89.5 | Green | 134 |
| Example 9 | | | | | | |
| Comparative | Z | M1 | 4.31 | 33.2 | Red | 105 |
| Example 10 | | | | | | |

From Experimental Example 3, it was identified that the organic light emitting devices of Examples 95 to 104 forming a light emitting layer using the compound according to the present disclosure as a single host material had superior light emission efficiency and lifetime compared to the organic light emitting devices of Comparative Examples 6 and 8 not using the compound according to the present disclosure as a single host material.

In addition, it was identified from Experimental Example 3 that the organic light emitting devices of Examples 105 to 119 forming a light emitting layer using a first host material corresponding to an n-host and the compound according to the present disclosure as a second host material corresponding to a p-host had superior light emission efficiency and lifetime compared to the organic light emitting devices of Comparative Examples 7, 9 and 10 forming a light emitting layer using a first host material corresponding to an n-host and a compound that is not the compound according to the present disclosure as a second host material corresponding to a p-host.

In addition, it was identified that the organic light emitting devices of Examples 95 to 104 forming a light emitting layer using the compound according to the present disclosure as a single host material exhibited equal or more superior performance in the light emission efficiency and the lifetime compared to the organic light emitting devices of Comparative Examples 6 and 8 forming a light emitting layer using a first host material corresponding to an n-host and a compound that is not the compound according to the present disclosure as a second host material corresponding to a p-host.

Considering such points, it is seen that an organic light emitting device has significantly improved light emission efficiency and lifetime when using the compound according to the present disclosure as a host material.

This is considered to be due to the fact that holes and electrons may be efficiently injected to a light emitting layer from each charge transfer layer when using the compound according to the present disclosure as a host material, and, as described above, due to orientation and space size formed by interactions between materials during deposition. In other words, this is considered to be effects obtained from the compound according to the present disclosure and differences in the orientation characteristics and space sizes between M1 and M2.

In addition, as seen from the results of Table 8, it is identified that effects of more superior efficiency and lifetime are obtained when including the compound represented by Chemical Formula 1 of the present disclosure (P type) and the compound represented by Chemical Formula 3 or Chemical Formula 4 of the present disclosure (N type) at the same time. This may lead to a forecast that an exciplex phenomenon occurs when including the two compounds at the same time.

The exciplex phenomenon is a phenomenon of releasing energy having sizes of a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and a driving voltage may be lowered, which resultantly helps with enhancement in the lifetime.

In the present disclosure, it was identified that superior device properties were obtained when, as the host of the light emitting layer, using the compound represented by Chemical Formula 1 as a donor role and the compound represented by Chemical Formula 3 or Chemical Formula 4 as an acceptor role.

The present disclosure is not limited to the above-described examples and may be prepared in various different forms, and those skilled in the art may understand that the present disclosure is embodied in other specific forms without changing technical ideas or essential characteristics of the present disclosure. Accordingly, it needs to be understood that the examples described above are for illustrative purposes only in all aspects and are not limitative.

### REFERENCE NUMERAL

100: Substrate
200: Positive electrode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Negative electrode

1. A heterocyclic compound represented by the following Chemical Formula 1: 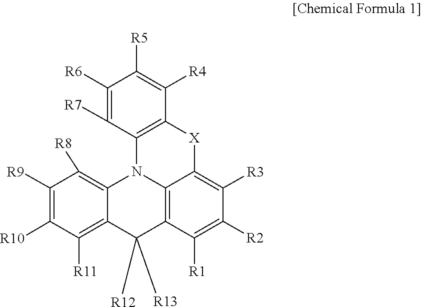
wherein, in Chemical Formula 1,
X is O or S;
R1 to R13 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and a group represented by the following Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group; and
at least one of R1 to R13 is a group represented by the following Chemical Formula 2, 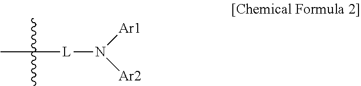
in Chemical Formula 2,
Ar1 and Ar2 are the same as or different from each other, and each independently a substituted or unsubstituted C6 to C60 aryl group; or a substituted or unsubstituted C2 to C60 heteroaryl group; and
L is a direct bond; a substituted or unsubstituted C6 to C60 arylene group; or a substituted or unsubstituted C2 to C60 heteroarylene group.

2. The heterocyclic compound of claim 1, wherein Chemical Formula 1 is represented by any one of the following Chemical Formula 1-1 to Chemical Formula 1-3: 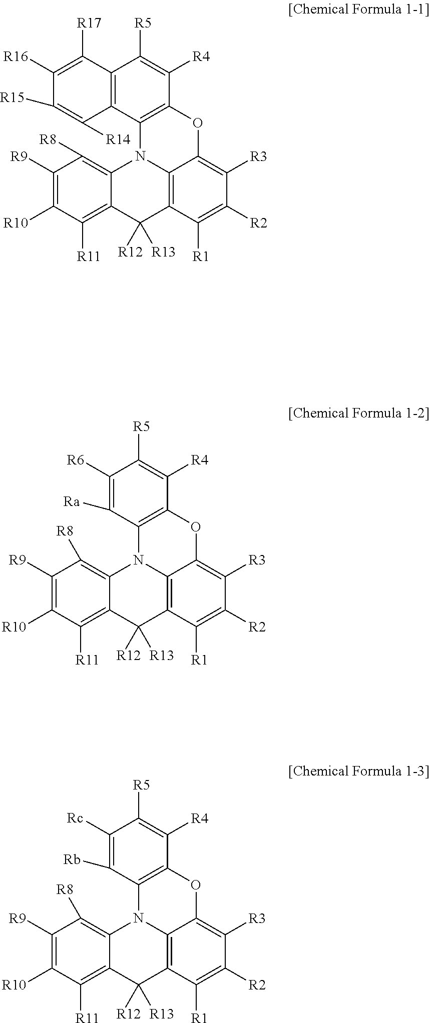
in Chemical Formula 1-1 to Chemical Formula 1-3,
R14 to R17 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and the group represented by Chemical Formula 2, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
Ra is a substituted or unsubstituted C1 to C60 alkyl group;
Rb and Re are the same as or different from each other, and each independently hydrogen; deuterium; a substituted or unsubstituted C6 to C60 aryl group; a substituted or unsubstituted C2 to C60 heteroaryl group; or the group represented by Chemical Formula 2; and
R1 to R6 and R8 to R13 have the same definitions as in Chemical Formula 1.

3. The heterocyclic compound of claim 1, wherein R12 and R13 are the same as or different from each other, and each independently a substituted or unsubstituted C1 to C60 alkyl group.

4. The heterocyclic compound of claim 2, wherein at least one of R1 to R3 and R9 to R11 in Chemical Formula 1-1 is the group represented by Chemical Formula 2.

5. The heterocyclic compound of claim 4, wherein, when at least one of R1 to R3 in Chemical Formula 1-1 is the group represented by Chemical Formula 2, at least one of R9 to R11 is a substituted or unsubstituted C6 to C60 aryl group; and
when at least one of R9 to R11 is the group represented by Chemical Formula 2, at least one of R1 to R3 is a substituted or unsubstituted C6 to C60 aryl group.

6. The heterocyclic compound of claim 2, wherein at least one of R1 to R6 and R9 to R11 in Chemical Formula 1-2 is the group represented by Chemical Formula 2.

7. The heterocyclic compound of claim 6, wherein, when at least one of R1 to R3 in Chemical Formula 1-2 is the group represented by Chemical Formula 2, at least one of R4 to R6 and R9 to R11 is a substituted or unsubstituted C6 to C60 aryl group;
when at least one of R4 to R6 is the group represented by Chemical Formula 2, at least one of R1 to R3 and R9 to R11 is a substituted or unsubstituted C6 to C60 aryl group; and
when at least one of R9 to R11 is the group represented by Chemical Formula 2, at least one of R1 to R6 is a substituted or unsubstituted C6 to C60 aryl group.

8. The heterocyclic compound of claim 2, wherein at least one of R1 to R7 and R9 to R11 in Chemical Formula 1-3 is the group represented by Chemical Formula 2.

9. The heterocyclic compound of claim 8, wherein, when at least one of R1 to R3 in Chemical Formula 1-3 is the group represented by Chemical Formula 2, at least one of R4 to R7 and R9 to R11 is a substituted or unsubstituted C6 to C60 aryl group;
when at least one of R4 to R7 is the group represented by Chemical Formula 2, at least one of R1 to R3 and R9 to R11 is a substituted or unsubstituted C6 to C60 aryl group; and
when at least one of R9 to R11 is the group represented by Chemical Formula 2, at least one of R1 to R7 is a substituted or unsubstituted C6 to C60 aryl group.

10. The heterocyclic compound of claim 1, wherein a deuterium content in Chemical Formula 1 is from 30% to 100% based on a total number of hydrogen atoms and deuterium atoms.

11. The heterocyclic compound of claim 1, wherein Chemical Formula 1 is represented by any one of the following compounds:

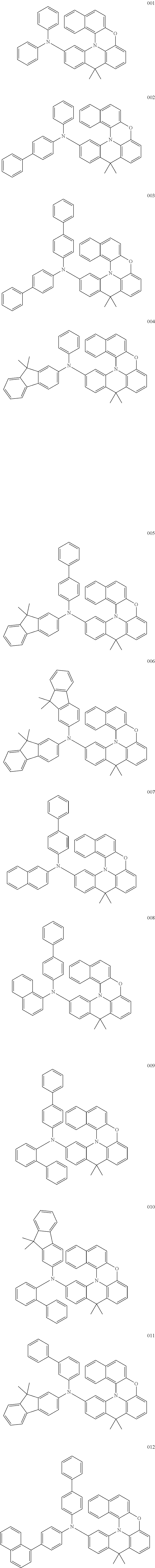

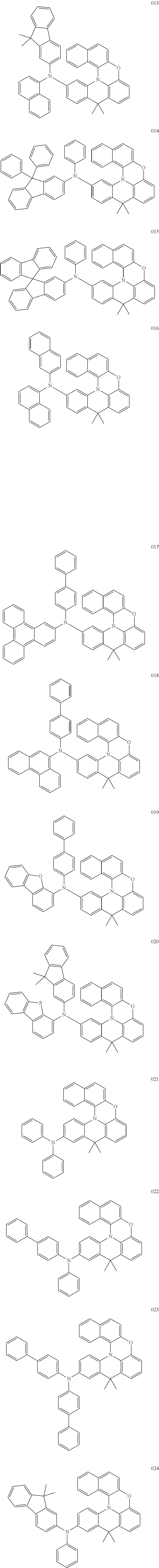

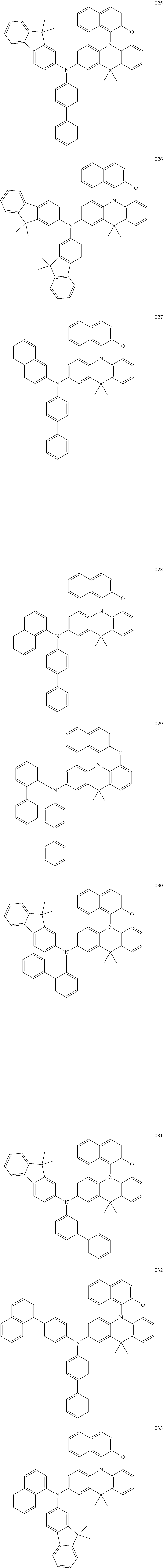

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

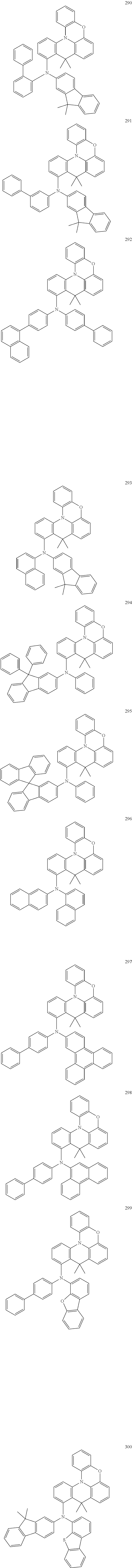

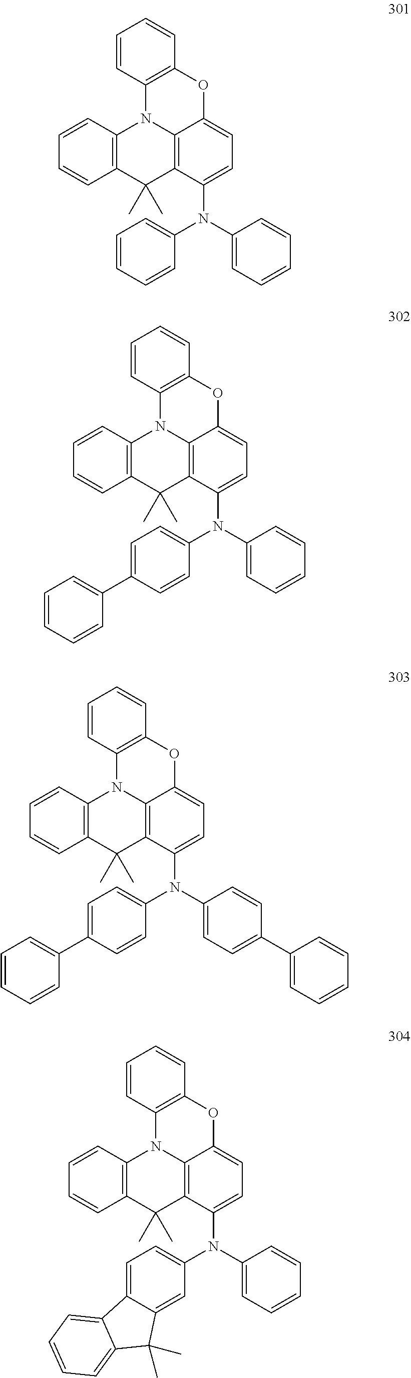

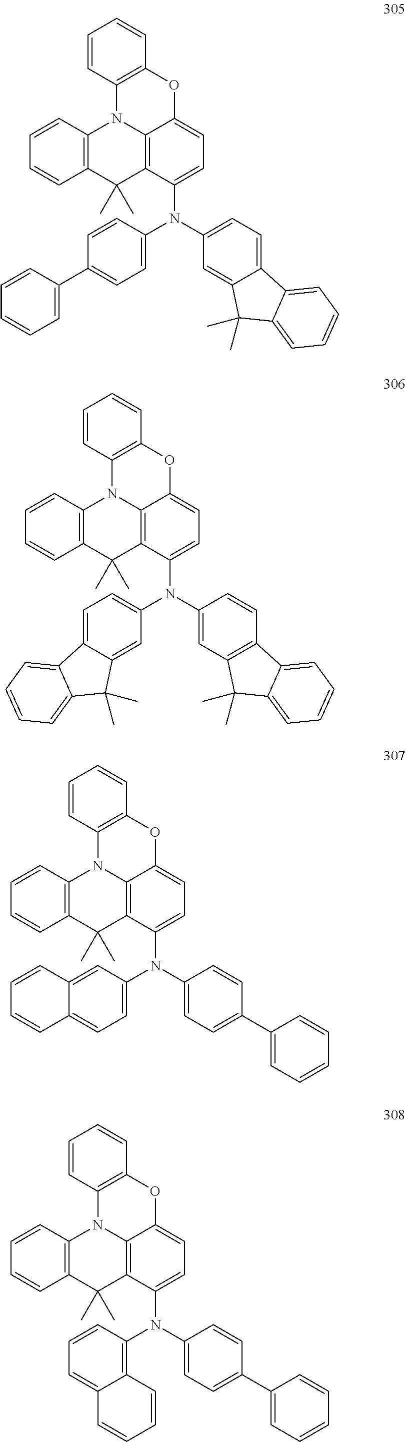

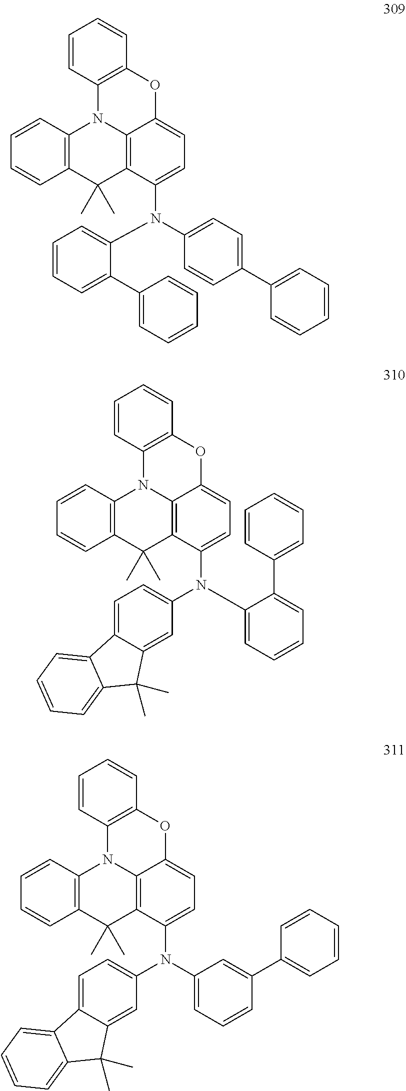 [SMILES: CC1(C)c2ccccc2N2c3ccccc3Oc3ccc(N(c4ccc(-c5ccccc5)cc4)c4ccccc4-c4ccccc4)c1c32 ; CC1(C)c2ccccc2-c2ccc(N(c3ccccc3-c3ccccc3)c3ccc4c5c3C(C)(C)c3ccccc3N5c3ccccc3O4)cc21 ; CC1(C)c2ccccc2-c2ccc(N(c3cccc(-c4ccccc4)c3)c3ccc4c5c3C(C)(C)c3ccccc3N5c3ccccc3O4)cc21]

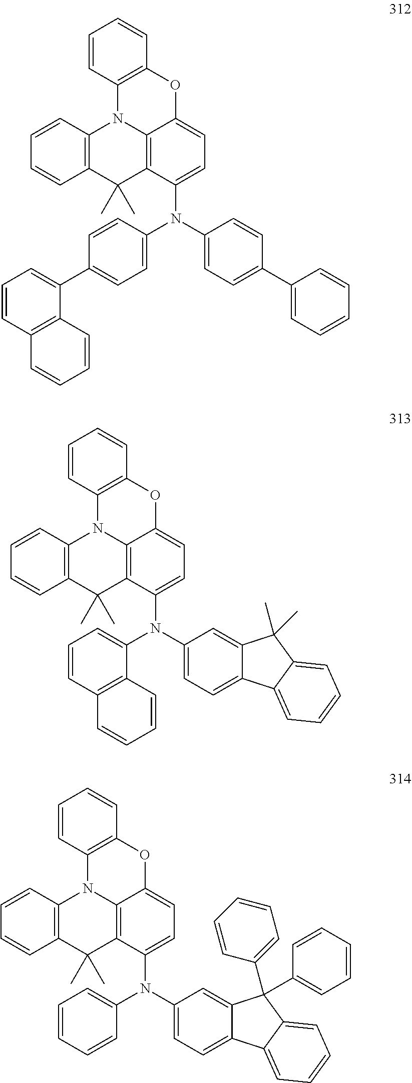 [SMILES: CC1(C)c2ccccc2N2c3ccccc3Oc3ccc(N(c4ccc(-c5ccccc5)cc4)c4ccc(-c5cccc6ccccc56)cc4)c1c32 ; CC1(C)c2ccccc2-c2ccc(N(c3ccc4c5c3C(C)(C)c3ccccc3N5c3ccccc3O4)c3cccc4ccccc34)cc21 ; CC1(C)c2ccccc2N2c3ccccc3Oc3ccc(N(c4ccccc4)c4ccc5c(c4)C(c4ccccc4)(c4ccccc4)c4ccccc4-5)c1c32]

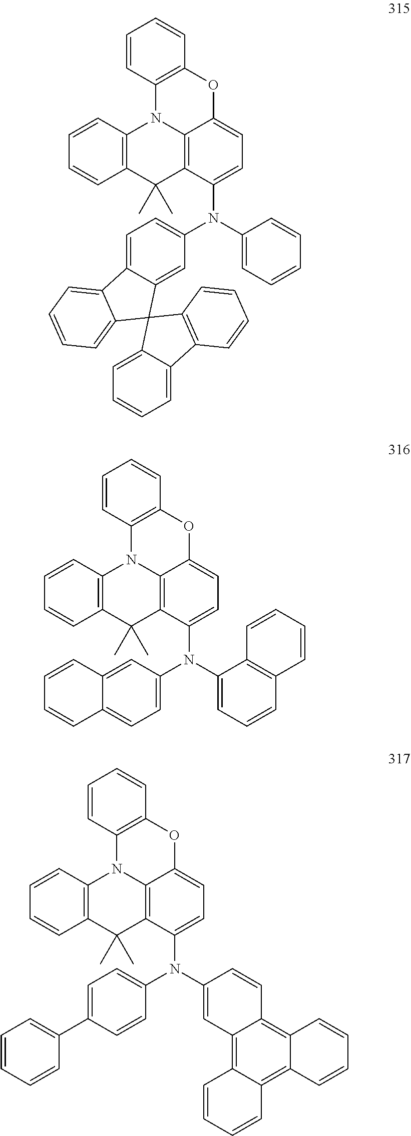

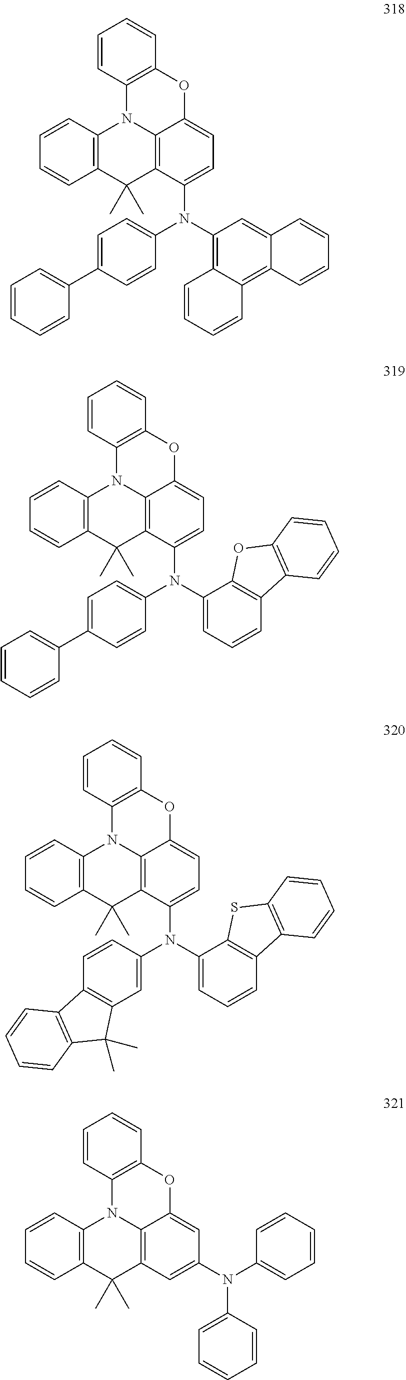

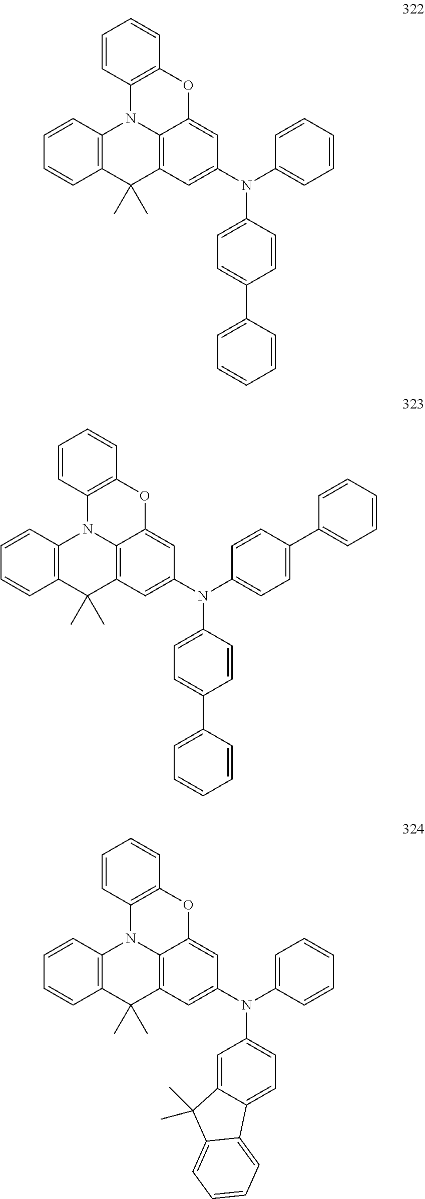 [SMILES: CC1(C)c2ccccc2N2c3ccccc3Oc3cc(N(c4ccccc4)c4ccc(-c5ccccc5)cc4)cc1c32 ; CC1(C)c2ccccc2N2c3ccccc3Oc3cc(N(c4ccc(-c5ccccc5)cc4)c4ccc(-c5ccccc5)cc4)cc1c32 ; CC1(C)c2ccccc2-c2ccc(N(c3ccccc3)c3cc4c5c(c3)C(C)(C)c3ccccc3N5c3ccccc3O4)cc21]

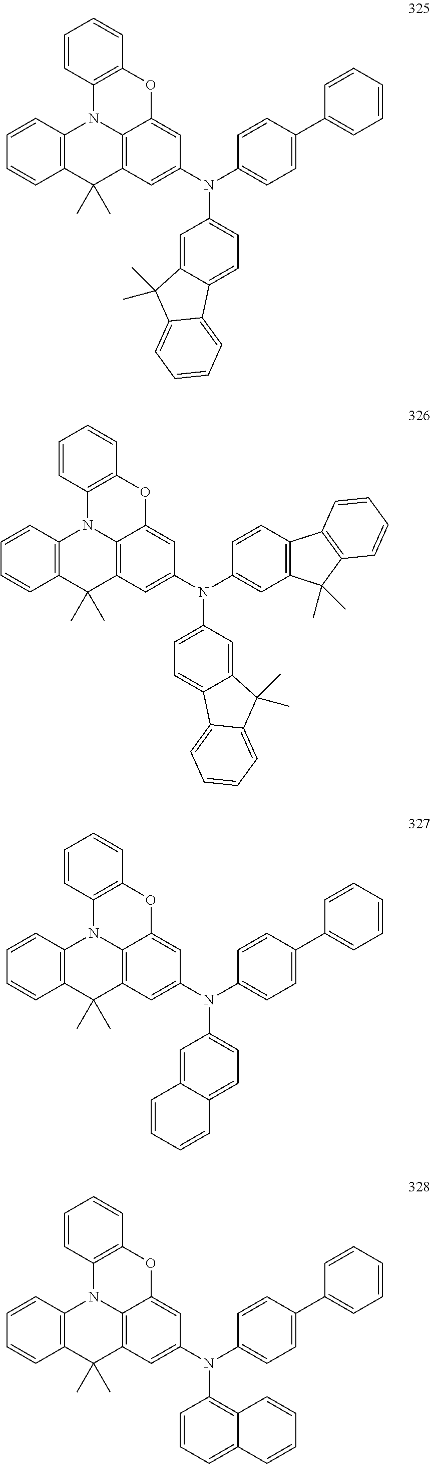 [SMILES: CC1(C)c2ccccc2-c2ccc(N(c3ccc(-c4ccccc4)cc3)c3cc4c5c(c3)C(C)(C)c3ccccc3N5c3ccccc3O4)cc21 ; CC1(C)c2ccccc2-c2ccc(N(c3ccc4c(c3)C(C)(C)c3ccccc3-4)c3cc4c5c(c3)C(C)(C)c3ccccc3N5c3ccccc3O4)cc21 ; CC1(C)c2ccccc2N2c3ccccc3Oc3cc(N(c4ccc(-c5ccccc5)cc4)c4ccc5ccccc5c4)cc1c32 ; CC1(C)c2ccccc2N2c3ccccc3Oc3cc(N(c4ccc(-c5ccccc5)cc4)c4cccc5ccccc45)cc1c32]

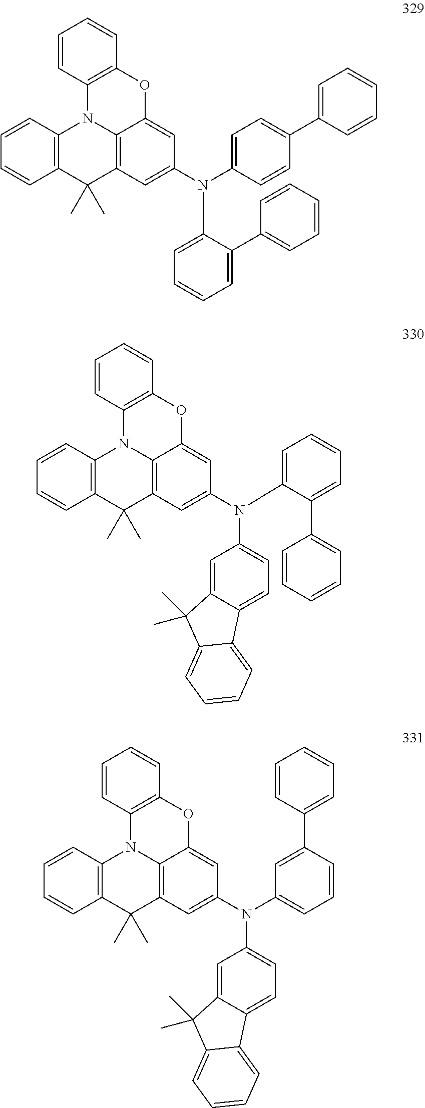 [SMILES: CC1(C)c2ccccc2N2c3ccccc3Oc3cc(N(c4ccc(-c5ccccc5)cc4)c4ccccc4-c4ccccc4)cc1c32 ; CC1(C)c2ccccc2-c2ccc(N(c3cc4c5c(c3)C(C)(C)c3ccccc3N5c3ccccc3O4)c3ccccc3-c3ccccc3)cc21 ; CC1(C)c2ccccc2-c2ccc(N(c3cccc(-c4ccccc4)c3)c3cc4c5c(c3)C(C)(C)c3ccccc3N5c3ccccc3O4)cc21]

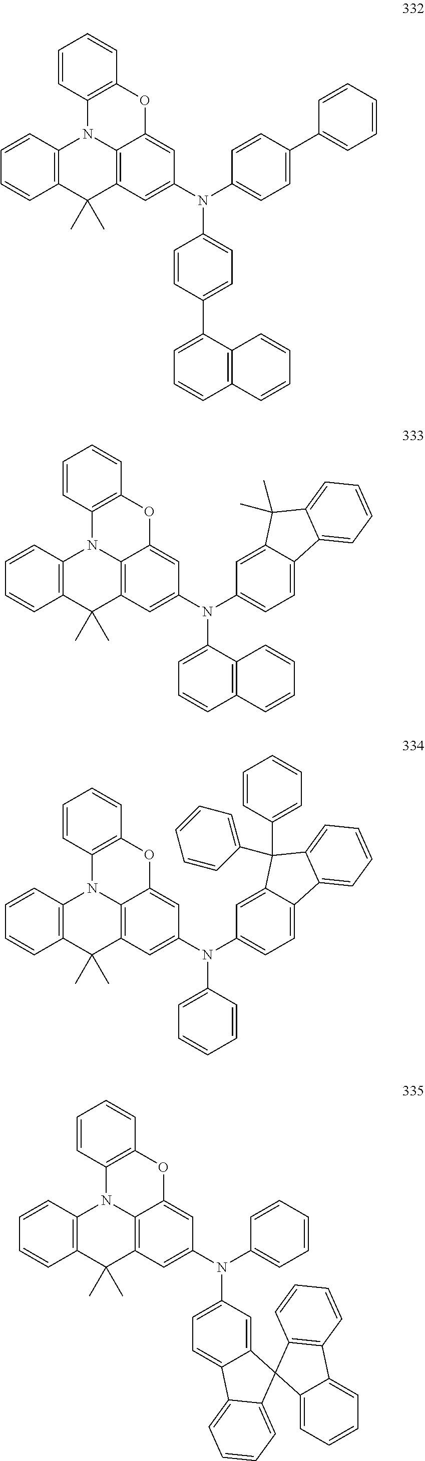

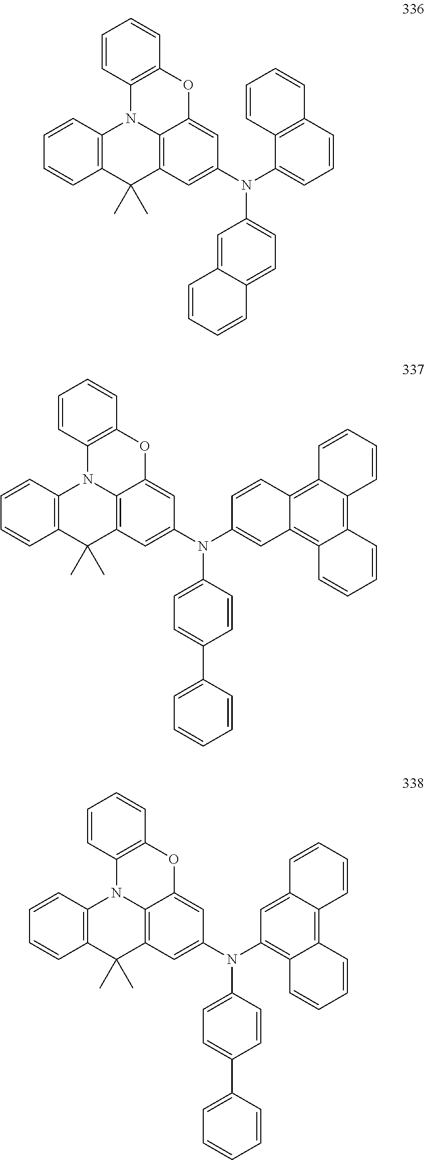 [SMILES: CC1(C)c2ccccc2N2c3ccccc3Oc3cc(N(c4ccc5ccccc5c4)c4cccc5ccccc45)cc1c32 ; CC1(C)c2ccccc2N2c3ccccc3Oc3cc(N(c4ccc(-c5ccccc5)cc4)c4ccc5c6ccccc6c6ccccc6c5c4)cc1c32 ; CC1(C)c2ccccc2N2c3ccccc3Oc3cc(N(c4ccc(-c5ccccc5)cc4)c4cc5ccccc5c5ccccc45)cc1c32]

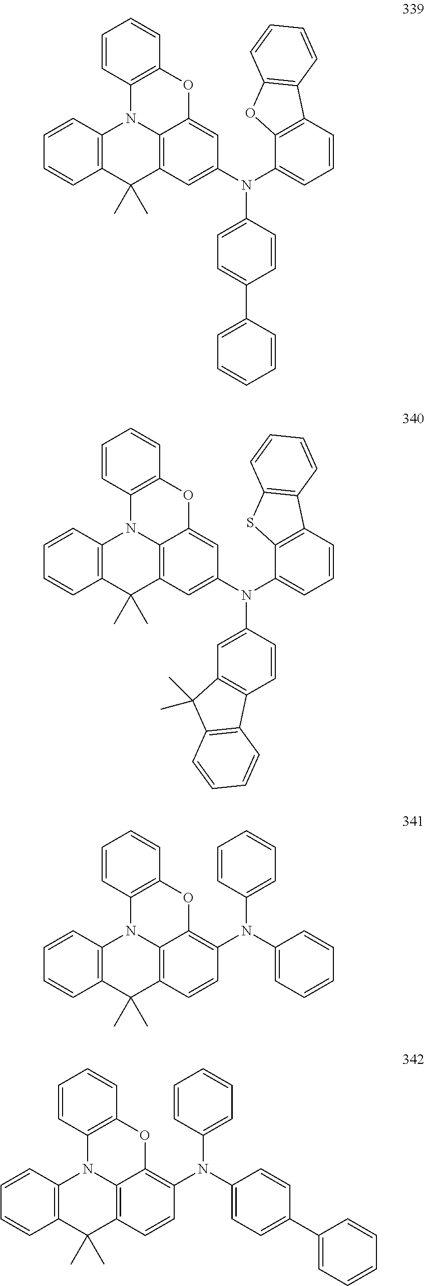

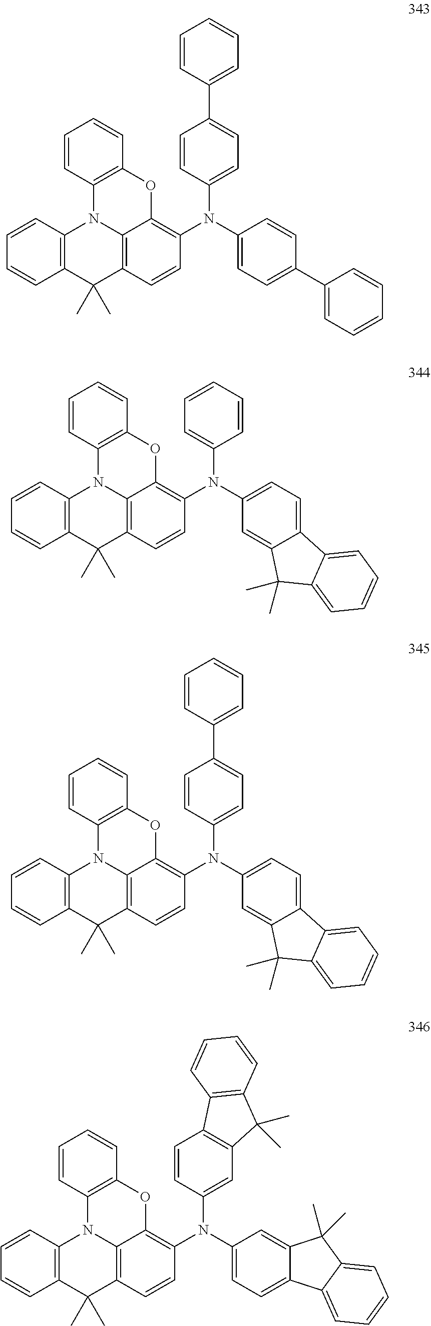 [SMILES: CC1(C)c2ccccc2N2c3ccccc3Oc3c(N(c4ccc(-c5ccccc5)cc4)c4ccc(-c5ccccc5)cc4)ccc1c32 ; CC1(C)c2ccccc2-c2ccc(N(c3ccccc3)c3ccc4c5c3Oc3ccccc3N5c3ccccc3C4(C)C)cc21 ; CC1(C)c2ccccc2-c2ccc(N(c3ccc(-c4ccccc4)cc3)c3ccc4c5c3Oc3ccccc3N5c3ccccc3C4(C)C)cc21 ; CC1(C)c2ccccc2-c2ccc(N(c3ccc4c(c3)C(C)(C)c3ccccc3-4)c3ccc4c5c3Oc3ccccc3N5c3ccccc3C4(C)C)cc21]

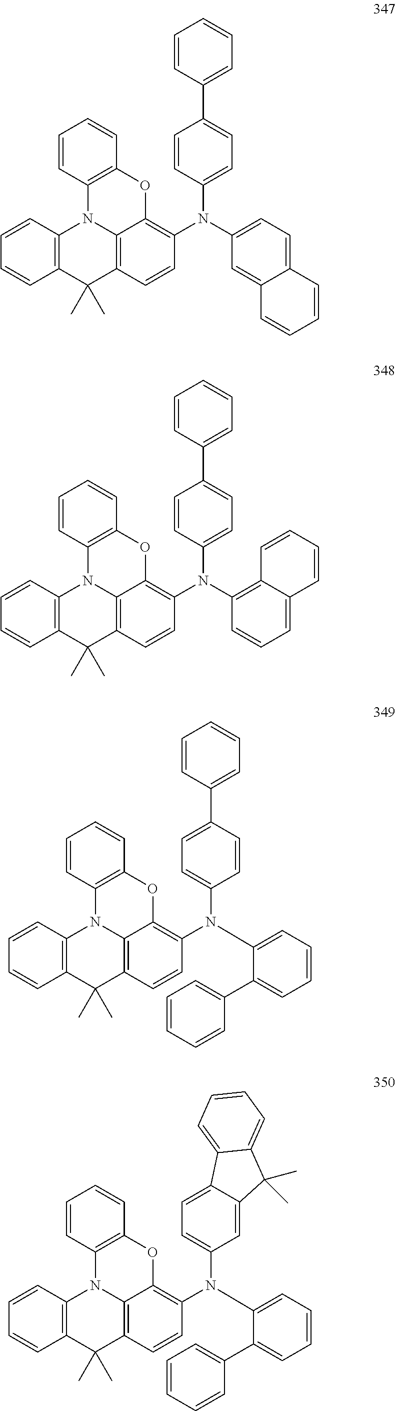 [SMILES: CC1(C)c2ccccc2N2c3ccccc3Oc3c(N(c4ccc(-c5ccccc5)cc4)c4ccc5ccccc5c4)ccc1c32 ; CC1(C)c2ccccc2N2c3ccccc3Oc3c(N(c4ccc(-c5ccccc5)cc4)c4cccc5ccccc45)ccc1c32 ; CC1(C)c2ccccc2N2c3ccccc3Oc3c(N(c4ccc(-c5ccccc5)cc4)c4ccccc4-c4ccccc4)ccc1c32 ; CC1(C)c2ccccc2-c2ccc(N(c3ccccc3-c3ccccc3)c3ccc4c5c3Oc3ccccc3N5c3ccccc3C4(C)C)cc21]

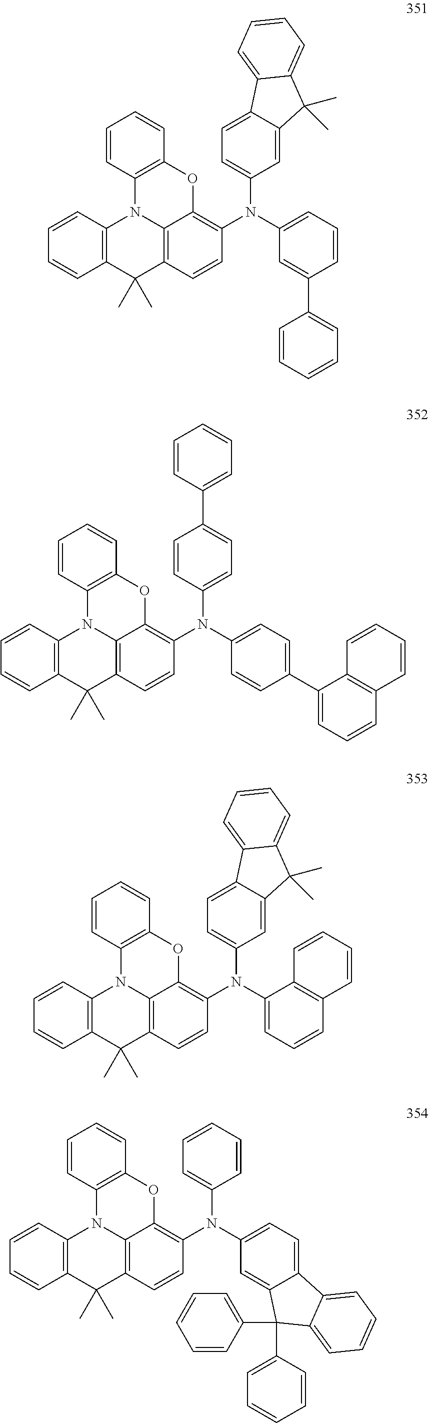

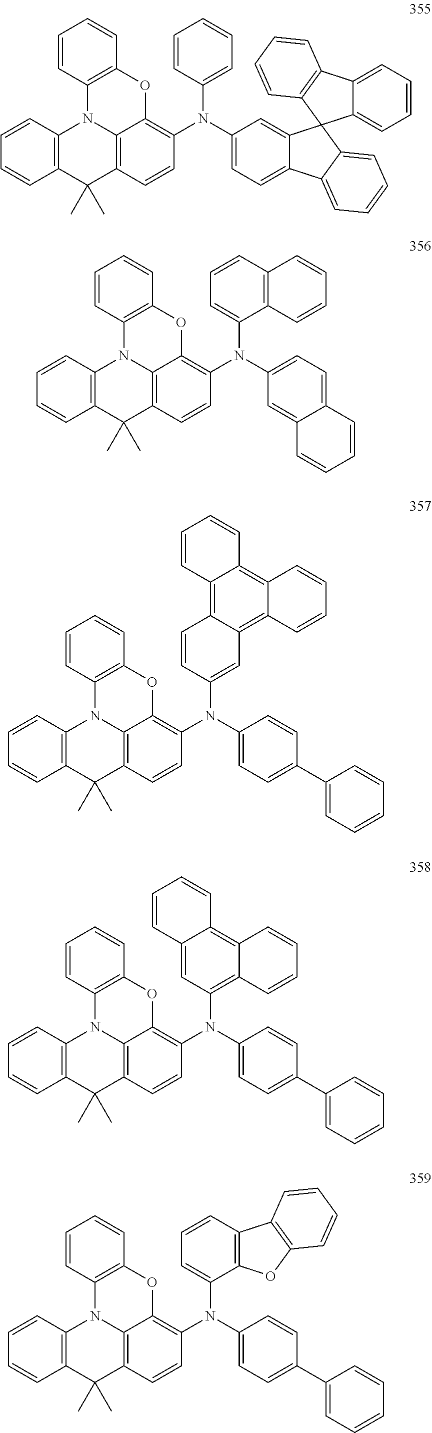

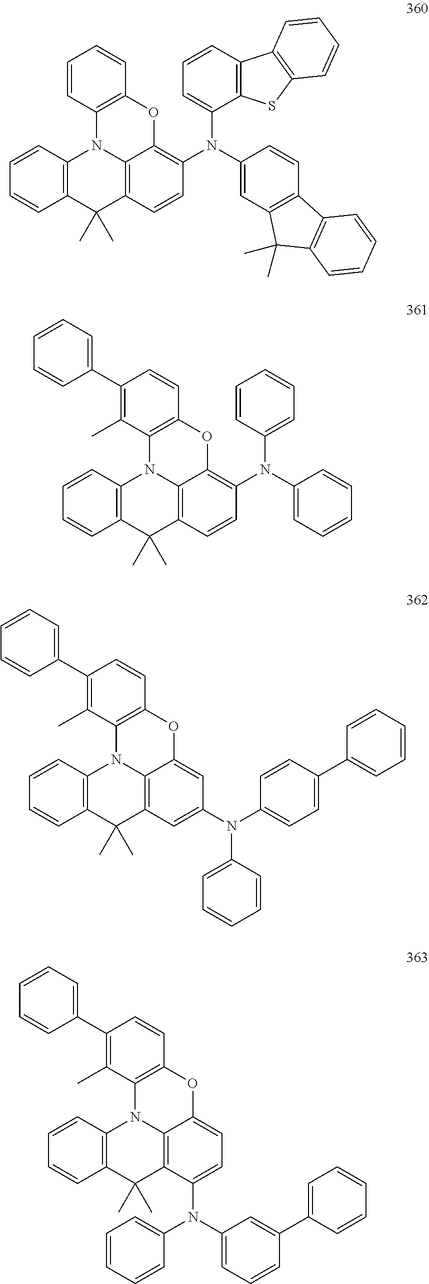 [SMILES: CC1(C)c2ccccc2-c2ccc(N(c3ccc4c5c3Oc3ccccc3N5c3ccccc3C4(C)C)c3cccc4c3sc3ccccc34)cc21 ; Cc1c(-c2ccccc2)ccc2c1N1c3ccccc3C(C)(C)c3ccc(N(c4ccccc4)c4ccccc4)c(c31)O2 ; Cc1c(-c2ccccc2)ccc2c1N1c3ccccc3C(C)(C)c3cc(N(c4ccccc4)c4ccc(-c5ccccc5)cc4)cc(c31)O2 ; Cc1c(-c2ccccc2)ccc2c1N1c3ccccc3C(C)(C)c3c(N(c4ccccc4)c4cccc(-c5ccccc5)c4)ccc(c31)O2]

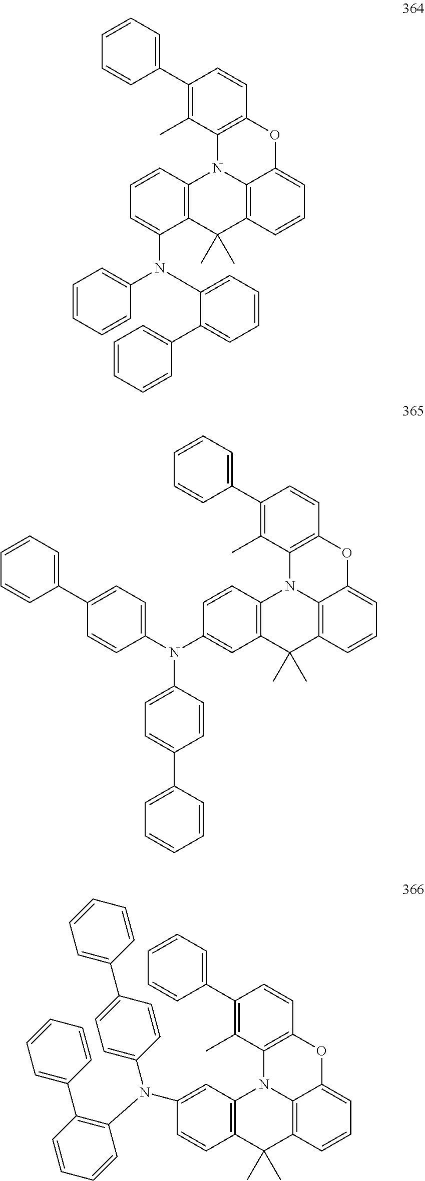 [SMILES: Cc1c(-c2ccccc2)ccc2c1N1c3cccc(N(c4ccccc4)c4ccccc4-c4ccccc4)c3C(C)(C)c3cccc(c31)O2 ; Cc1c(-c2ccccc2)ccc2c1N1c3ccc(N(c4ccc(-c5ccccc5)cc4)c4ccc(-c5ccccc5)cc4)cc3C(C)(C)c3cccc(c31)O2 ; Cc1c(-c2ccccc2)ccc2c1N1c3cc(N(c4ccc(-c5ccccc5)cc4)c4ccccc4-c4ccccc4)ccc3C(C)(C)c3cccc(c31)O2]

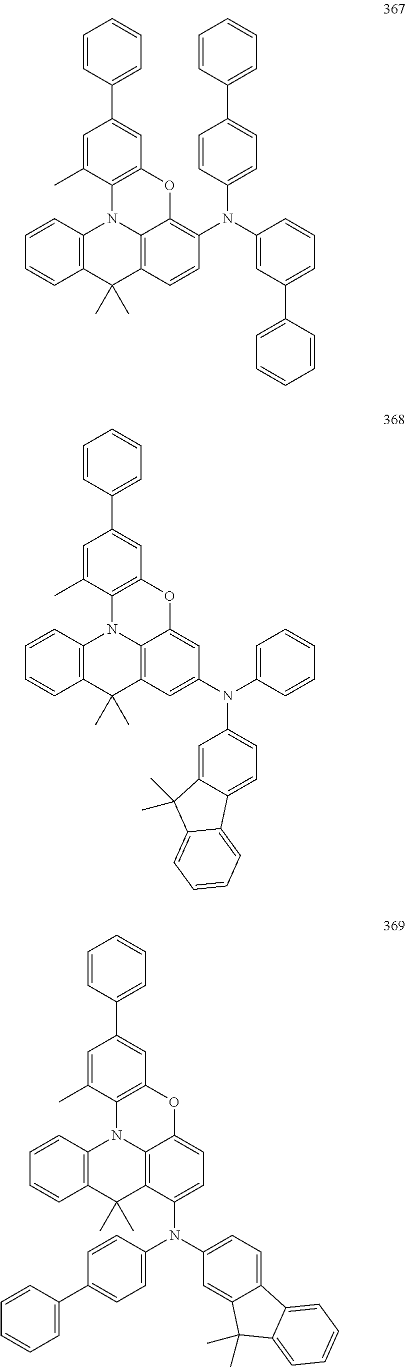 [SMILES: Cc1cc(-c2ccccc2)cc2c1N1c3ccccc3C(C)(C)c3ccc(N(c4ccc(-c5ccccc5)cc4)c4cccc(-c5ccccc5)c4)c(c31)O2 ; Cc1cc(-c2ccccc2)cc2c1N1c3ccccc3C(C)(C)c3cc(N(c4ccccc4)c4ccc5c(c4)C(C)(C)c4ccccc4-5)cc(c31)O2 ; Cc1cc(-c2ccccc2)cc2c1N1c3ccccc3C(C)(C)c3c(N(c4ccc(-c5ccccc5)cc4)c4ccc5c(c4)C(C)(C)c4ccccc4-5)ccc(c31)O2]

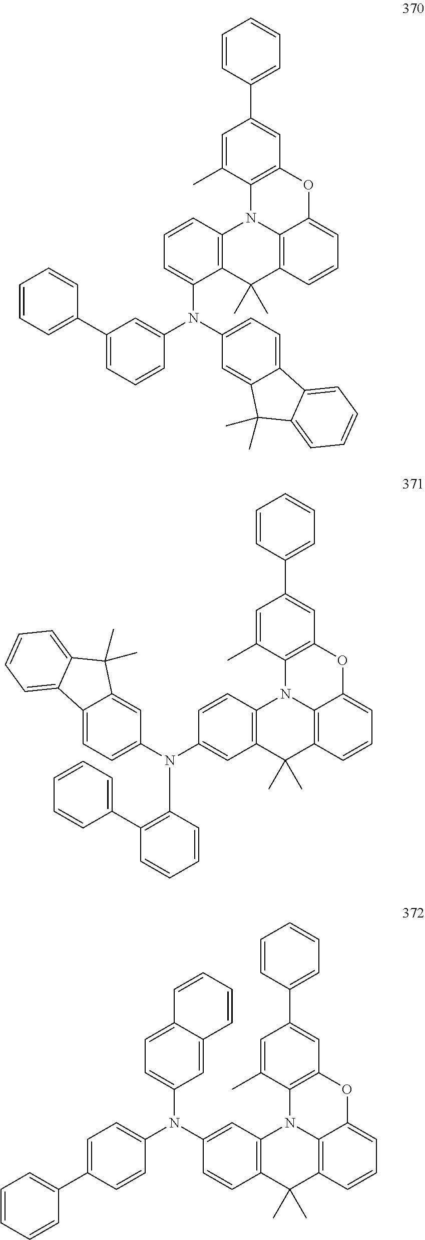 [SMILES: Cc1cc(-c2ccccc2)cc2c1N1c3cccc(N(c4cccc(-c5ccccc5)c4)c4ccc5c(c4)C(C)(C)c4ccccc4-5)c3C(C)(C)c3cccc(c31)O2 ; Cc1cc(-c2ccccc2)cc2c1N1c3ccc(N(c4ccc5c(c4)C(C)(C)c4ccccc4-5)c4ccccc4-c4ccccc4)cc3C(C)(C)c3cccc(c31)O2 ; Cc1cc(-c2ccccc2)cc2c1N1c3cc(N(c4ccc(-c5ccccc5)cc4)c4ccc5ccccc5c4)ccc3C(C)(C)c3cccc(c31)O2]

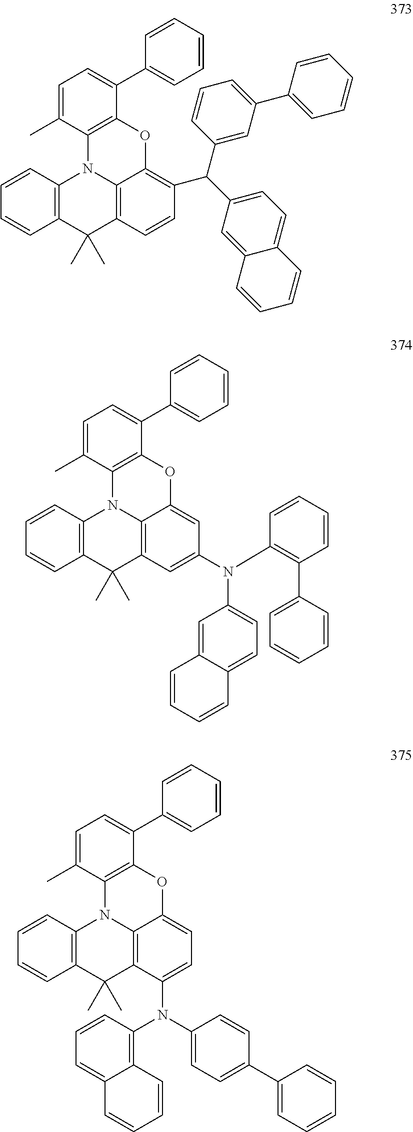

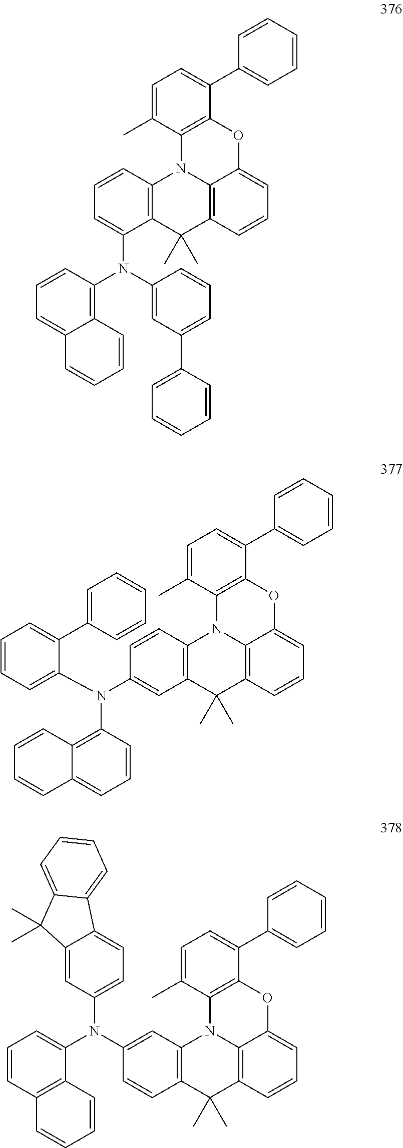

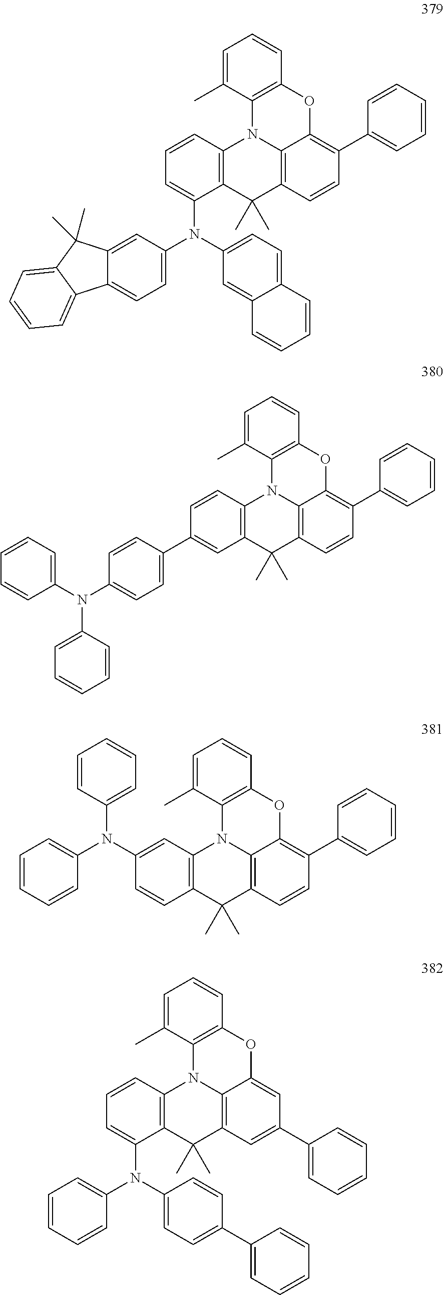

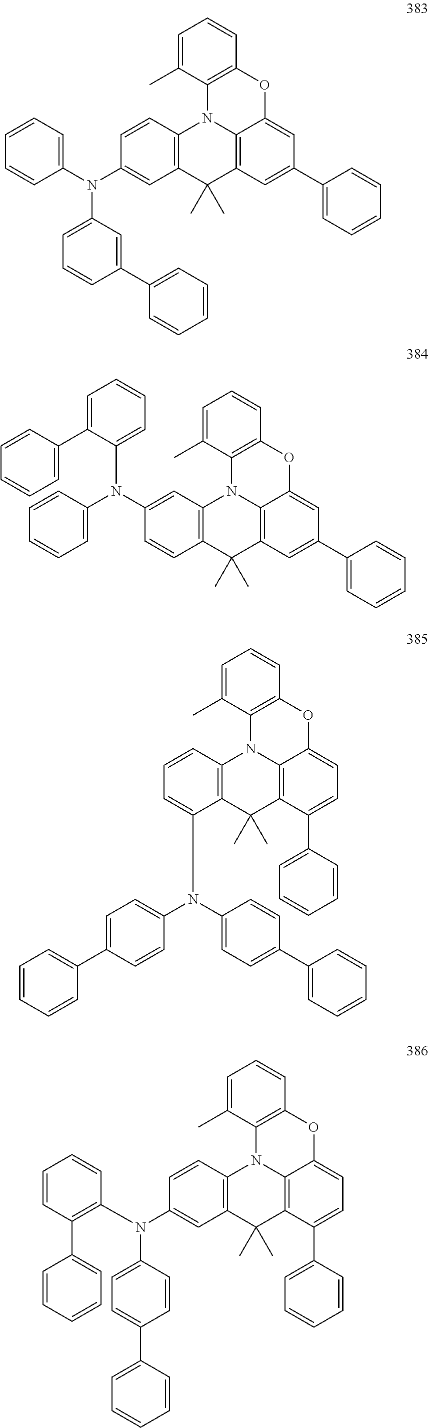 [SMILES: Cc1cccc2c1N1c3ccc(N(c4ccccc4)c4cccc(-c5ccccc5)c4)cc3C(C)(C)c3cc(-c4ccccc4)cc(c31)O2 ; Cc1cccc2c1N1c3cc(N(c4ccccc4)c4ccccc4-c4ccccc4)ccc3C(C)(C)c3cc(-c4ccccc4)cc(c31)O2 ; Cc1cccc2c1N1c3cccc(N(c4ccc(-c5ccccc5)cc4)c4ccc(-c5ccccc5)cc4)c3C(C)(C)c3c(-c4ccccc4)ccc(c31)O2 ; Cc1cccc2c1N1c3ccc(N(c4ccc(-c5ccccc5)cc4)c4ccccc4-c4ccccc4)cc3C(C)(C)c3c(-c4ccccc4)ccc(c31)O2]

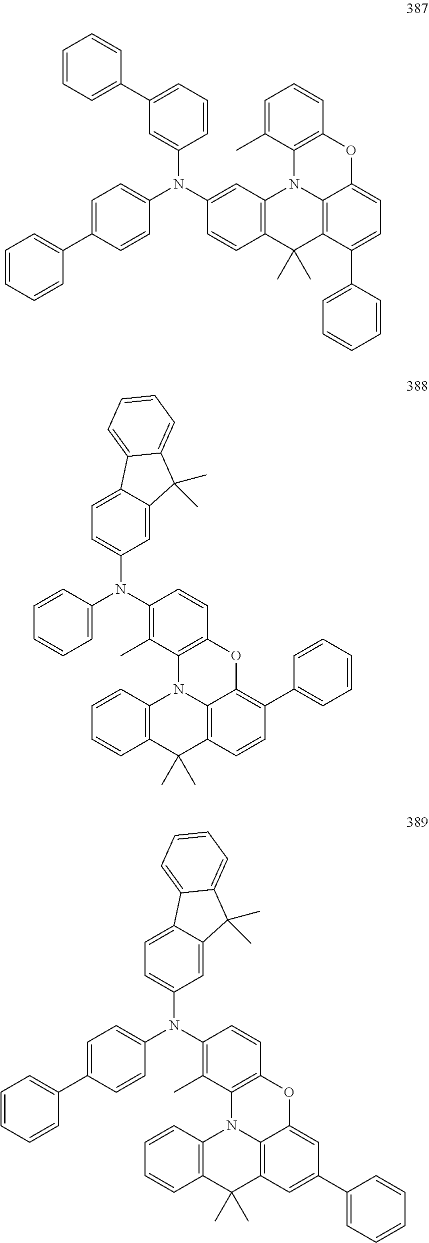 [SMILES: Cc1cccc2c1N1c3cc(N(c4ccc(-c5ccccc5)cc4)c4cccc(-c5ccccc5)c4)ccc3C(C)(C)c3c(-c4ccccc4)ccc(c31)O2 ; Cc1c(N(c2ccccc2)c2ccc3c(c2)C(C)(C)c2ccccc2-3)ccc2c1N1c3ccccc3C(C)(C)c3ccc(-c4ccccc4)c(c31)O2 ; Cc1c(N(c2ccc(-c3ccccc3)cc2)c2ccc3c(c2)C(C)(C)c2ccccc2-3)ccc2c1N1c3ccccc3C(C)(C)c3cc(-c4ccccc4)cc(c31)O2]

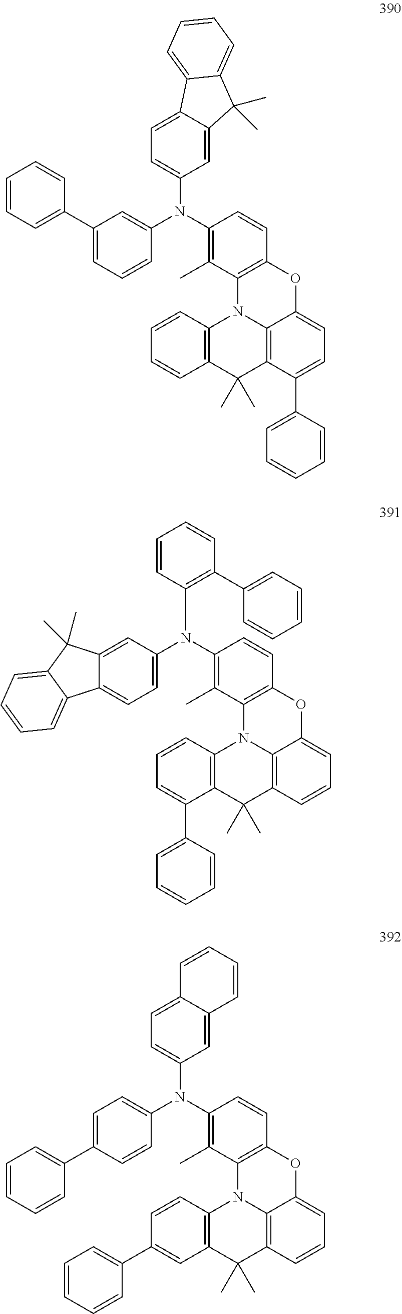 [SMILES: Cc1c(N(c2cccc(-c3ccccc3)c2)c2ccc3c(c2)C(C)(C)c2ccccc2-3)ccc2c1N1c3ccccc3C(C)(C)c3c(-c4ccccc4)ccc(c31)O2 ; Cc1c(N(c2ccc3c(c2)C(C)(C)c2ccccc2-3)c2ccccc2-c2ccccc2)ccc2c1N1c3cccc(-c4ccccc4)c3C(C)(C)c3cccc(c31)O2 ; Cc1c(N(c2ccc(-c3ccccc3)cc2)c2ccc3ccccc3c2)ccc2c1N1c3ccc(-c4ccccc4)cc3C(C)(C)c3cccc(c31)O2]

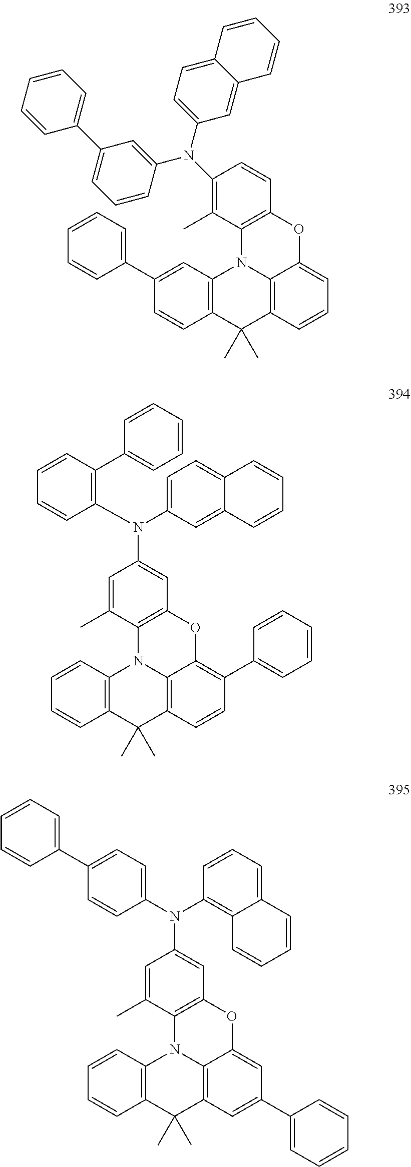 [SMILES: Cc1c(N(c2cccc(-c3ccccc3)c2)c2ccc3ccccc3c2)ccc2c1N1c3cc(-c4ccccc4)ccc3C(C)(C)c3cccc(c31)O2 ; Cc1cc(N(c2ccc3ccccc3c2)c2ccccc2-c2ccccc2)cc2c1N1c3ccccc3C(C)(C)c3ccc(-c4ccccc4)c(c31)O2 ; Cc1cc(N(c2ccc(-c3ccccc3)cc2)c2cccc3ccccc23)cc2c1N1c3ccccc3C(C)(C)c3cc(-c4ccccc4)cc(c31)O2]

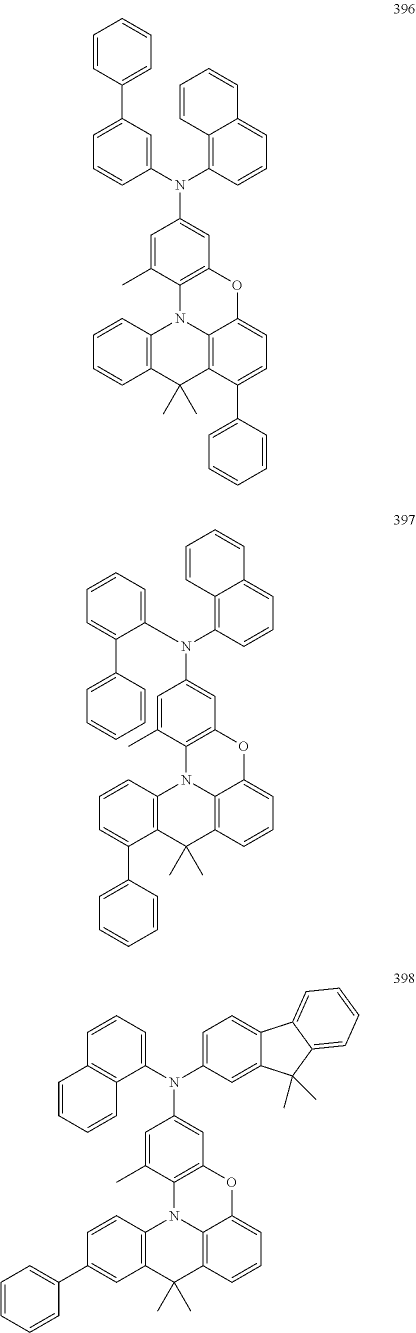 [SMILES: Cc1cc(N(c2cccc(-c3ccccc3)c2)c2cccc3ccccc23)cc2c1N1c3ccccc3C(C)(C)c3c(-c4ccccc4)ccc(c31)O2 ; Cc1cc(N(c2ccccc2-c2ccccc2)c2cccc3ccccc23)cc2c1N1c3cccc(-c4ccccc4)c3C(C)(C)c3cccc(c31)O2 ; Cc1cc(N(c2ccc3c(c2)C(C)(C)c2ccccc2-3)c2cccc3ccccc23)cc2c1N1c3ccc(-c4ccccc4)cc3C(C)(C)c3cccc(c31)O2]

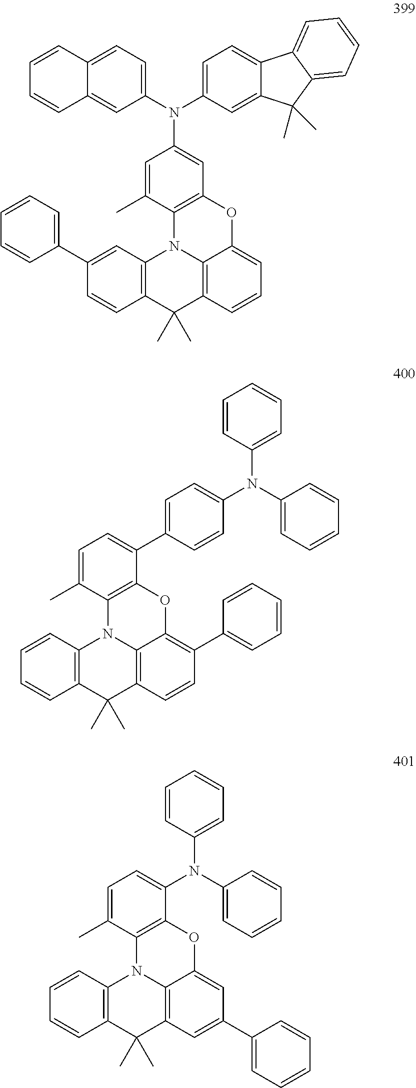 [SMILES: Cc1cc(N(c2ccc3c(c2)C(C)(C)c2ccccc2-3)c2ccc3ccccc3c2)cc2c1N1c3cc(-c4ccccc4)ccc3C(C)(C)c3cccc(c31)O2 ; Cc1ccc(-c2ccc(N(c3ccccc3)c3ccccc3)cc2)c2c1N1c3ccccc3C(C)(C)c3ccc(-c4ccccc4)c(c31)O2 ; Cc1ccc(N(c2ccccc2)c2ccccc2)c2c1N1c3ccccc3C(C)(C)c3cc(-c4ccccc4)cc(c31)O2]

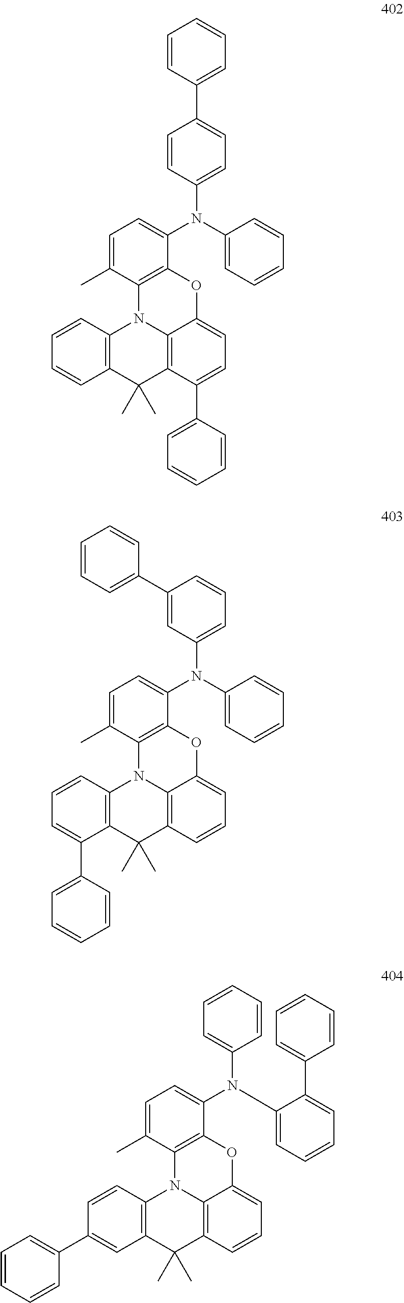 [SMILES: Cc1ccc(N(c2ccccc2)c2ccc(-c3ccccc3)cc2)c2c1N1c3ccccc3C(C)(C)c3c(-c4ccccc4)ccc(c31)O2 ; Cc1ccc(N(c2ccccc2)c2cccc(-c3ccccc3)c2)c2c1N1c3cccc(-c4ccccc4)c3C(C)(C)c3cccc(c31)O2 ; Cc1ccc(N(c2ccccc2)c2ccccc2-c2ccccc2)c2c1N1c3ccc(-c4ccccc4)cc3C(C)(C)c3cccc(c31)O2]

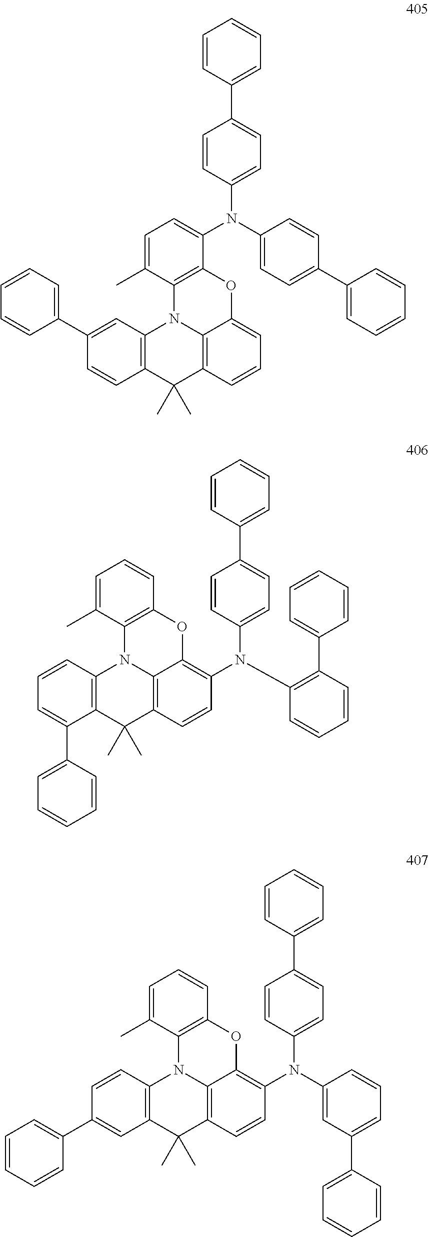 [SMILES: Cc1ccc(N(c2ccc(-c3ccccc3)cc2)c2ccc(-c3ccccc3)cc2)c2c1N1c3cc(-c4ccccc4)ccc3C(C)(C)c3cccc(c31)O2 ; Cc1cccc2c1N1c3cccc(-c4ccccc4)c3C(C)(C)c3ccc(N(c4ccc(-c5ccccc5)cc4)c4ccccc4-c4ccccc4)c(c31)O2 ; Cc1cccc2c1N1c3ccc(-c4ccccc4)cc3C(C)(C)c3ccc(N(c4ccc(-c5ccccc5)cc4)c4cccc(-c5ccccc5)c4)c(c31)O2]

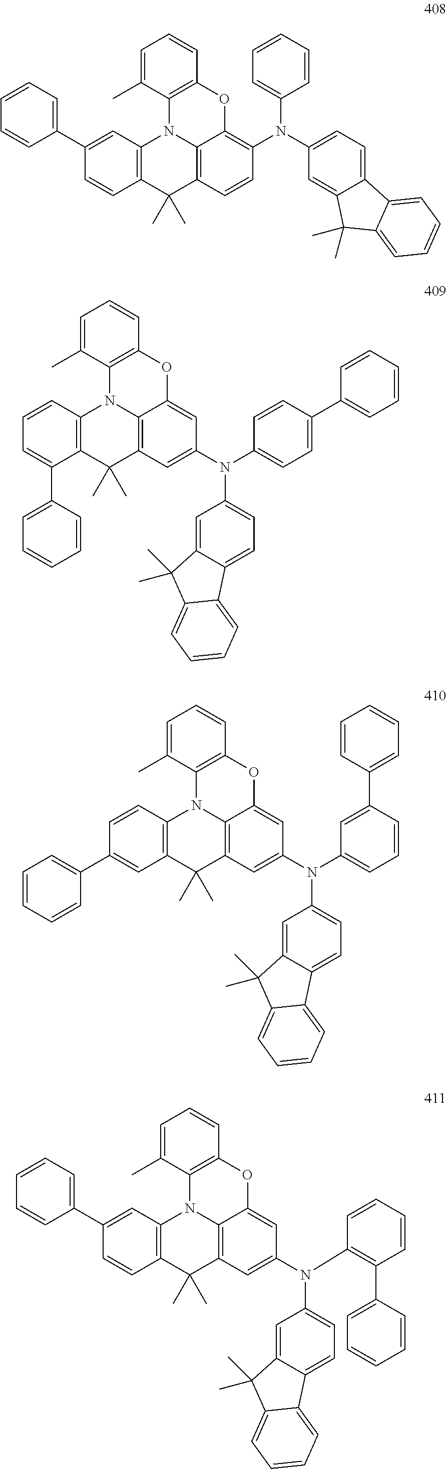

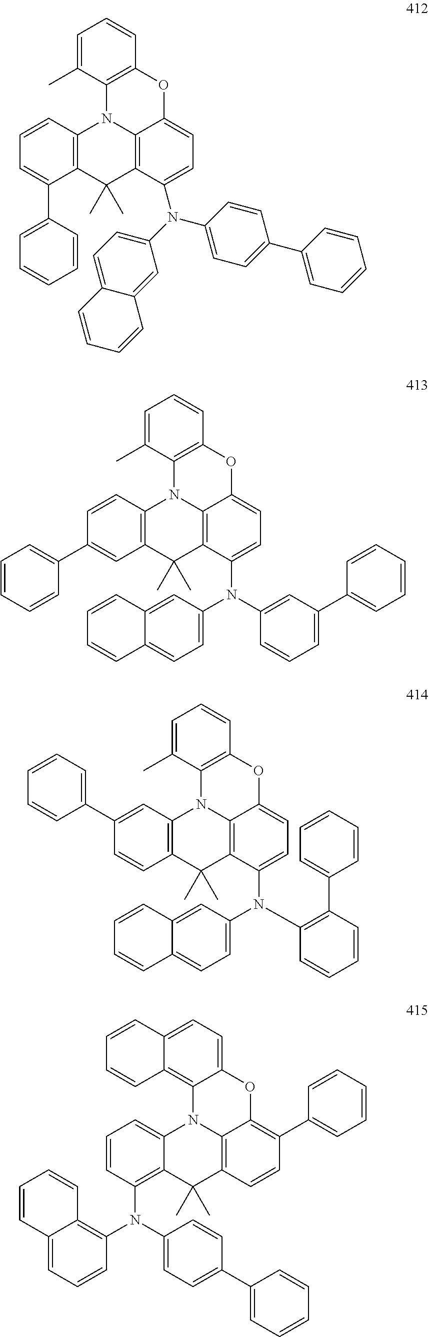 [SMILES: Cc1cccc2c1N1c3cccc(-c4ccccc4)c3C(C)(C)c3c(N(c4ccc(-c5ccccc5)cc4)c4ccc5ccccc5c4)ccc(c31)O2 ; Cc1cccc2c1N1c3ccc(-c4ccccc4)cc3C(C)(C)c3c(N(c4cccc(-c5ccccc5)c4)c4ccc5ccccc5c4)ccc(c31)O2 ; Cc1cccc2c1N1c3cc(-c4ccccc4)ccc3C(C)(C)c3c(N(c4ccc5ccccc5c4)c4ccccc4-c4ccccc4)ccc(c31)O2 ; CC1(C)c2ccc(-c3ccccc3)c3c2N(c2cccc(N(c4ccc(-c5ccccc5)cc4)c4cccc5ccccc45)c21)c1c(ccc2ccccc12)O3]

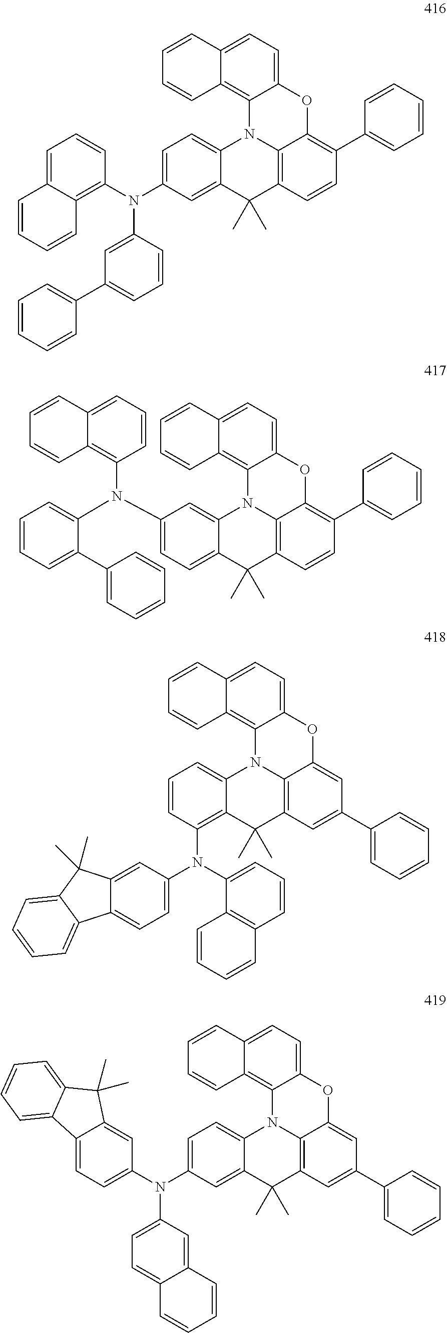 [SMILES: CC1(C)c2cc(N(c3cccc(-c4ccccc4)c3)c3cccc4ccccc34)ccc2N2c3c1ccc(-c1ccccc1)c3Oc1ccc3ccccc3c12 ; CC1(C)c2ccc(N(c3ccccc3-c3ccccc3)c3cccc4ccccc34)cc2N2c3c1ccc(-c1ccccc1)c3Oc1ccc3ccccc3c12 ; CC1(C)c2ccccc2-c2ccc(N(c3cccc4c3C(C)(C)c3cc(-c5ccccc5)cc5c3N4c3c(ccc4ccccc34)O5)c3cccc4ccccc34)cc21 ; CC1(C)c2ccccc2-c2ccc(N(c3ccc4c(c3)C(C)(C)c3cc(-c5ccccc5)cc5c3N4c3c(ccc4ccccc34)O5)c3ccc4ccccc4c3)cc21]

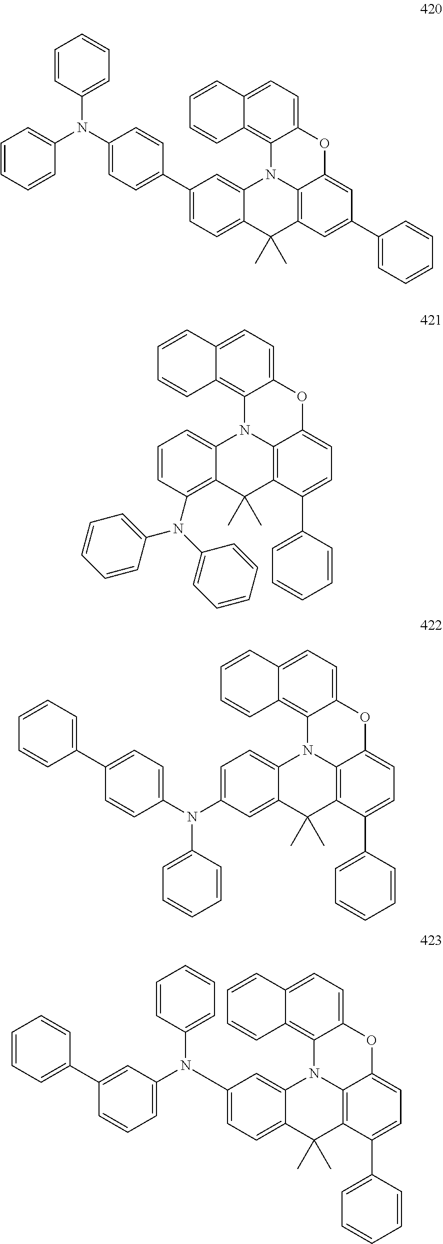 [SMILES: CC1(C)c2ccc(-c3ccc(N(c4ccccc4)c4ccccc4)cc3)cc2N2c3c(cc(-c4ccccc4)cc31)Oc1ccc3ccccc3c12 ; CC1(C)c2c(N(c3ccccc3)c3ccccc3)cccc2N2c3c(ccc(-c4ccccc4)c31)Oc1ccc3ccccc3c12 ; CC1(C)c2cc(N(c3ccccc3)c3ccc(-c4ccccc4)cc3)ccc2N2c3c(ccc(-c4ccccc4)c31)Oc1ccc3ccccc3c12 ; CC1(C)c2ccc(N(c3ccccc3)c3cccc(-c4ccccc4)c3)cc2N2c3c(ccc(-c4ccccc4)c31)Oc1ccc3ccccc3c12]

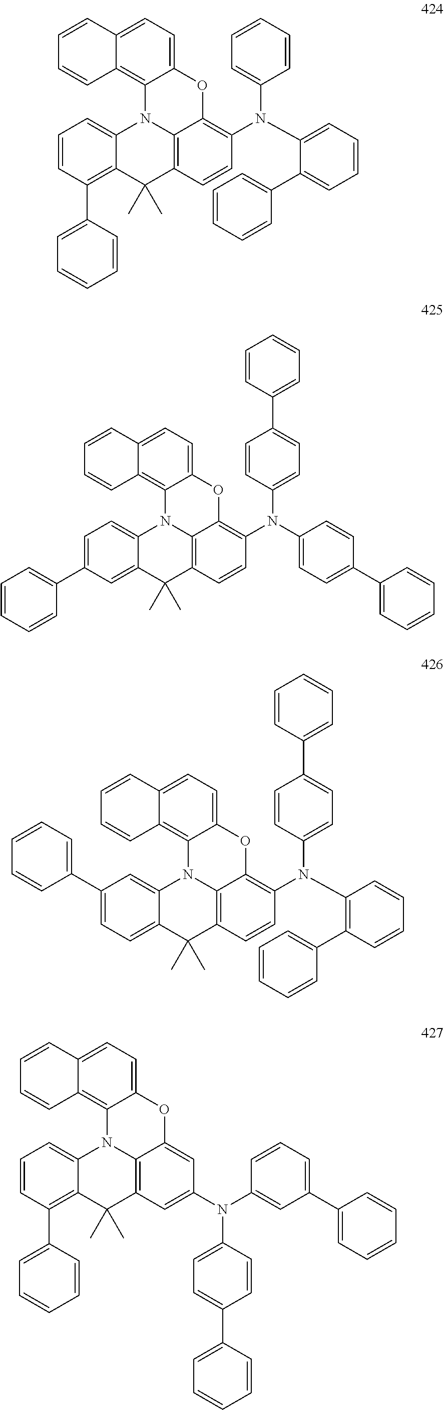 [SMILES: CC1(C)c2ccc(N(c3ccccc3)c3ccccc3-c3ccccc3)c3c2N(c2cccc(-c4ccccc4)c21)c1c(ccc2ccccc12)O3 ; CC1(C)c2cc(-c3ccccc3)ccc2N2c3c1ccc(N(c1ccc(-c4ccccc4)cc1)c1ccc(-c4ccccc4)cc1)c3Oc1ccc3ccccc3c12 ; CC1(C)c2ccc(-c3ccccc3)cc2N2c3c1ccc(N(c1ccc(-c4ccccc4)cc1)c1ccccc1-c1ccccc1)c3Oc1ccc3ccccc3c12 ; CC1(C)c2cc(N(c3ccc(-c4ccccc4)cc3)c3cccc(-c4ccccc4)c3)cc3c2N(c2cccc(-c4ccccc4)c21)c1c(ccc2ccccc12)O3]

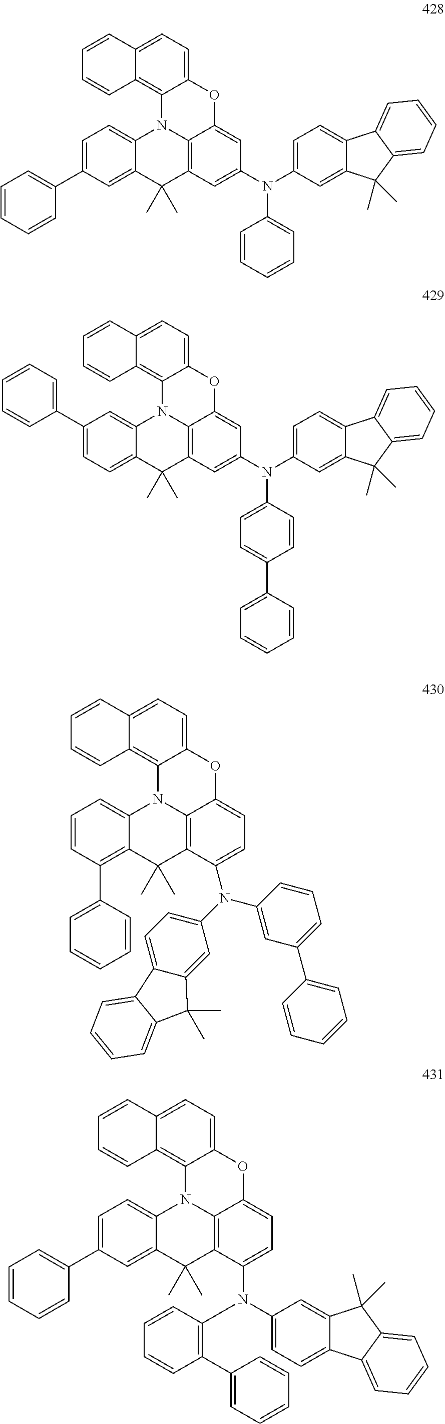

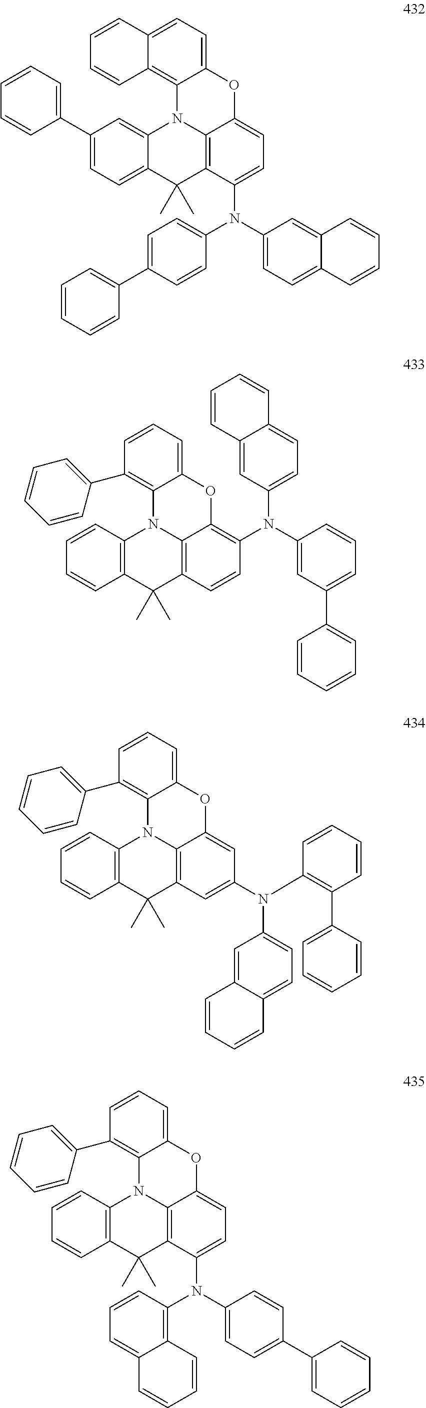 [SMILES: CC1(C)c2ccc(-c3ccccc3)cc2N2c3c(ccc(N(c4ccc(-c5ccccc5)cc4)c4ccc5ccccc5c4)c31)Oc1ccc3ccccc3c12 ; CC1(C)c2ccccc2N2c3c(cccc3-c3ccccc3)Oc3c(N(c4cccc(-c5ccccc5)c4)c4ccc5ccccc5c4)ccc1c32 ; CC1(C)c2ccccc2N2c3c(cccc3-c3ccccc3)Oc3cc(N(c4ccc5ccccc5c4)c4ccccc4-c4ccccc4)cc1c32 ; CC1(C)c2ccccc2N2c3c(cccc3-c3ccccc3)Oc3ccc(N(c4ccc(-c5ccccc5)cc4)c4cccc5ccccc45)c1c32]

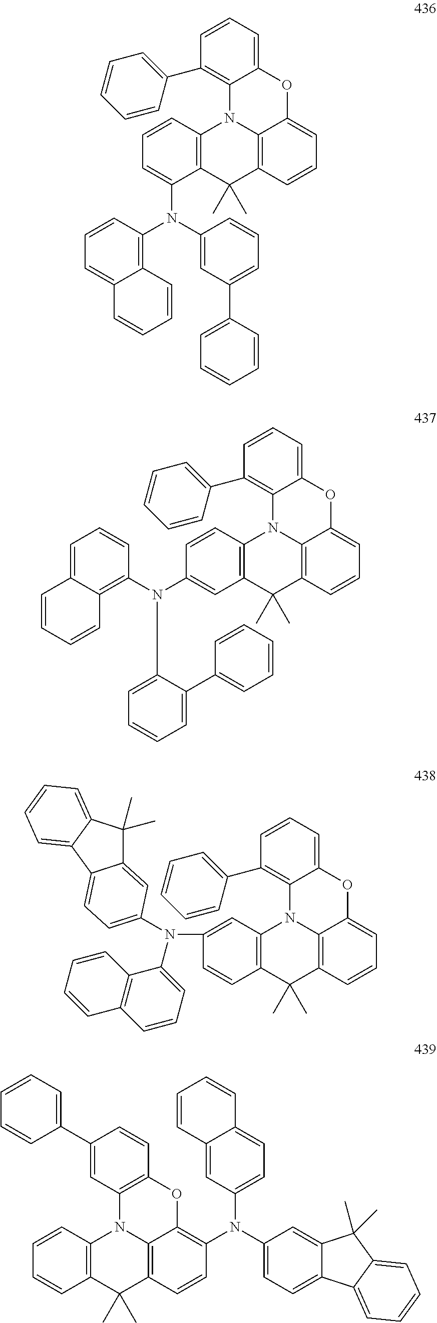 [SMILES: CC1(C)c2cccc3c2N(c2cccc(N(c4cccc(-c5ccccc5)c4)c4cccc5ccccc45)c21)c1c(cccc1-c1ccccc1)O3 ; CC1(C)c2cc(N(c3ccccc3-c3ccccc3)c3cccc4ccccc34)ccc2N2c3c(cccc3-c3ccccc3)Oc3cccc1c32 ; CC1(C)c2ccccc2-c2ccc(N(c3ccc4c(c3)N3c5c(cccc5-c5ccccc5)Oc5cccc(c53)C4(C)C)c3cccc4ccccc34)cc21 ; CC1(C)c2ccccc2-c2ccc(N(c3ccc4ccccc4c3)c3ccc4c5c3Oc3ccc(-c6ccccc6)cc3N5c3ccccc3C4(C)C)cc21]

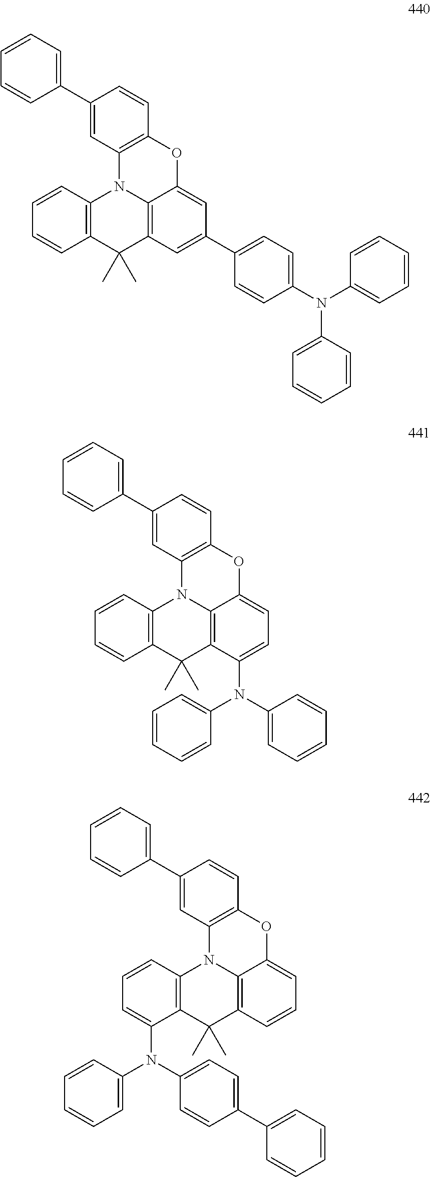 [SMILES: CC1(C)c2ccccc2N2c3cc(-c4ccccc4)ccc3Oc3cc(-c4ccc(N(c5ccccc5)c5ccccc5)cc4)cc1c32 ; CC1(C)c2ccccc2N2c3cc(-c4ccccc4)ccc3Oc3ccc(N(c4ccccc4)c4ccccc4)c1c32 ; CC1(C)c2cccc3c2N(c2cc(-c4ccccc4)ccc2O3)c2cccc(N(c3ccccc3)c3ccc(-c4ccccc4)cc3)c21]

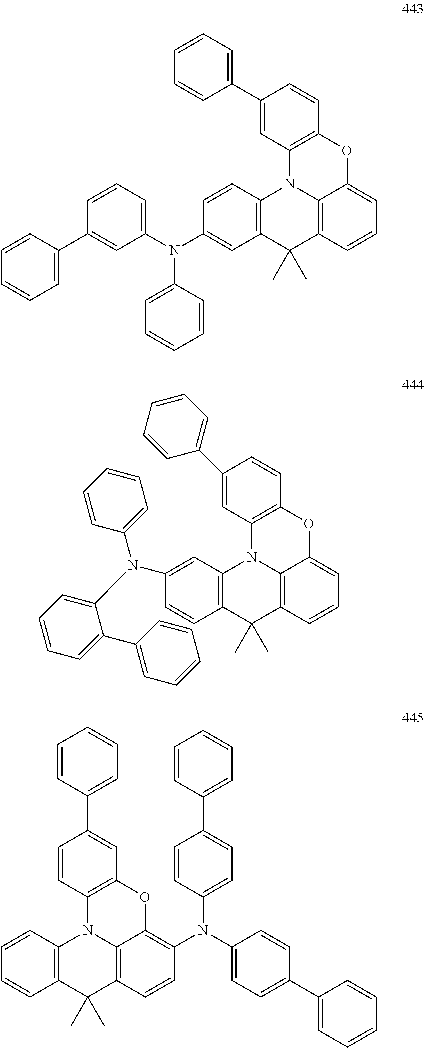

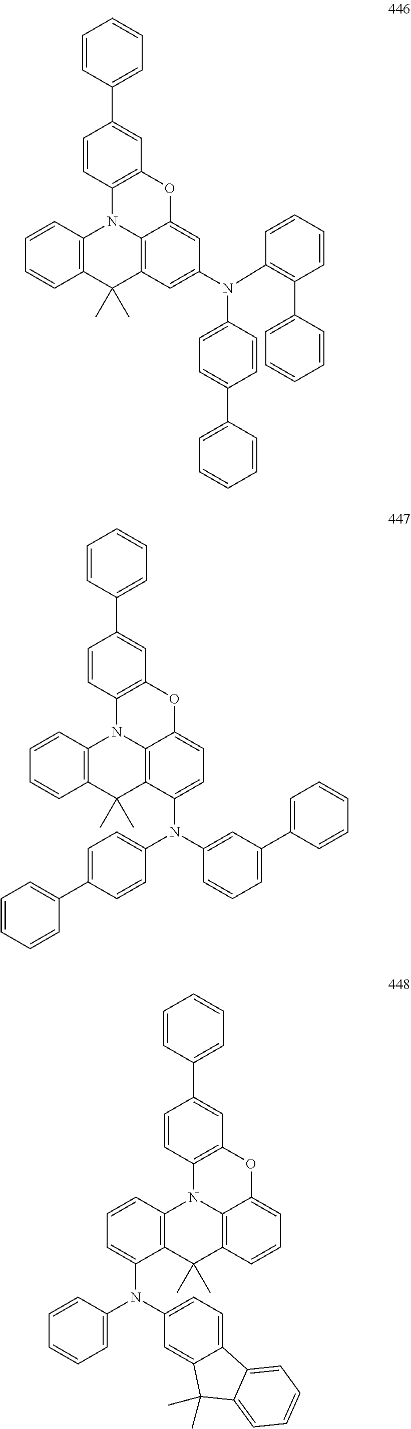 [SMILES: CC1(C)c2ccccc2N2c3ccc(-c4ccccc4)cc3Oc3cc(N(c4ccc(-c5ccccc5)cc4)c4ccccc4-c4ccccc4)cc1c32 ; CC1(C)c2ccccc2N2c3ccc(-c4ccccc4)cc3Oc3ccc(N(c4ccc(-c5ccccc5)cc4)c4cccc(-c5ccccc5)c4)c1c32 ; CC1(C)c2ccccc2-c2ccc(N(c3ccccc3)c3cccc4c3C(C)(C)c3cccc5c3N4c3ccc(-c4ccccc4)cc3O5)cc21]

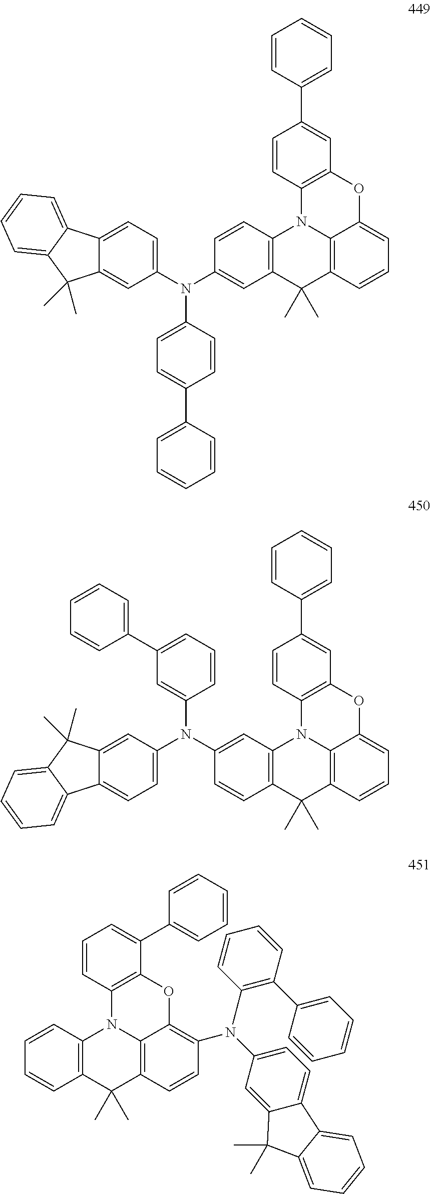 [SMILES: CC1(C)c2ccccc2-c2ccc(N(c3ccc(-c4ccccc4)cc3)c3ccc4c(c3)C(C)(C)c3cccc5c3N4c3ccc(-c4ccccc4)cc3O5)cc21 ; CC1(C)c2ccccc2-c2ccc(N(c3cccc(-c4ccccc4)c3)c3ccc4c(c3)N3c5ccc(-c6ccccc6)cc5Oc5cccc(c53)C4(C)C)cc21 ; CC1(C)c2ccccc2-c2ccc(N(c3ccccc3-c3ccccc3)c3ccc4c5c3Oc3c(-c6ccccc6)cccc3N5c3ccccc3C4(C)C)cc21]

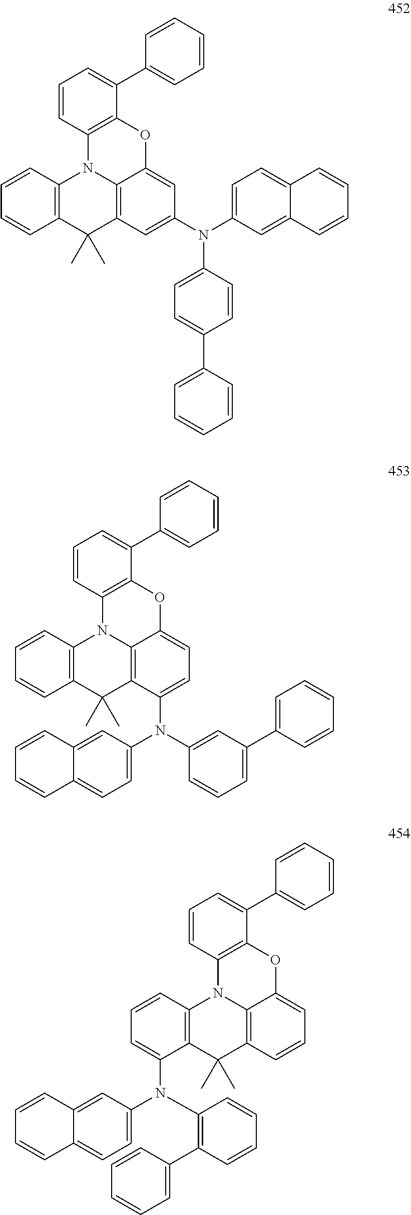 [SMILES: CC1(C)c2ccccc2N2c3cccc(-c4ccccc4)c3Oc3cc(N(c4ccc(-c5ccccc5)cc4)c4ccc5ccccc5c4)cc1c32 ; CC1(C)c2ccccc2N2c3cccc(-c4ccccc4)c3Oc3ccc(N(c4cccc(-c5ccccc5)c4)c4ccc5ccccc5c4)c1c32 ; CC1(C)c2cccc3c2N(c2cccc(-c4ccccc4)c2O3)c2cccc(N(c3ccc4ccccc4c3)c3ccccc3-c3ccccc3)c21]

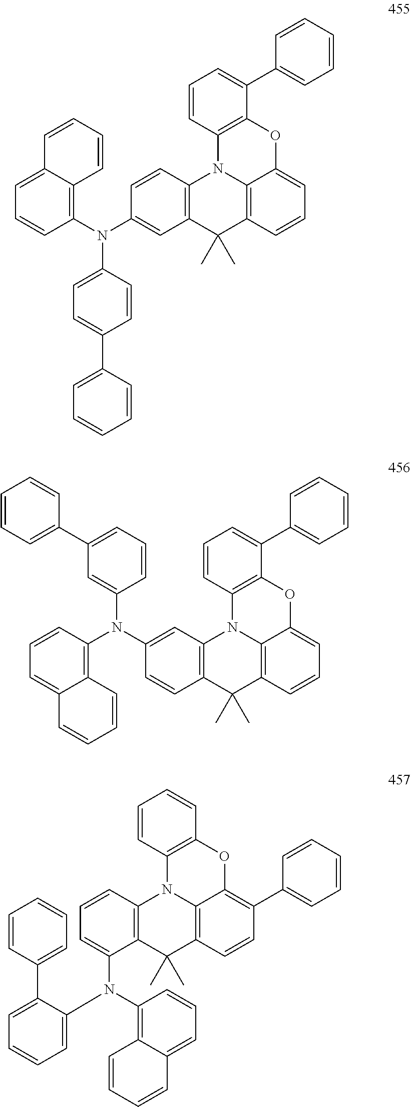 [SMILES: CC1(C)c2cc(N(c3ccc(-c4ccccc4)cc3)c3cccc4ccccc34)ccc2N2c3cccc(-c4ccccc4)c3Oc3cccc1c32 ; CC1(C)c2ccc(N(c3cccc(-c4ccccc4)c3)c3cccc4ccccc34)cc2N2c3cccc(-c4ccccc4)c3Oc3cccc1c32 ; CC1(C)c2ccc(-c3ccccc3)c3c2N(c2ccccc2O3)c2cccc(N(c3ccccc3-c3ccccc3)c3cccc4ccccc34)c21]

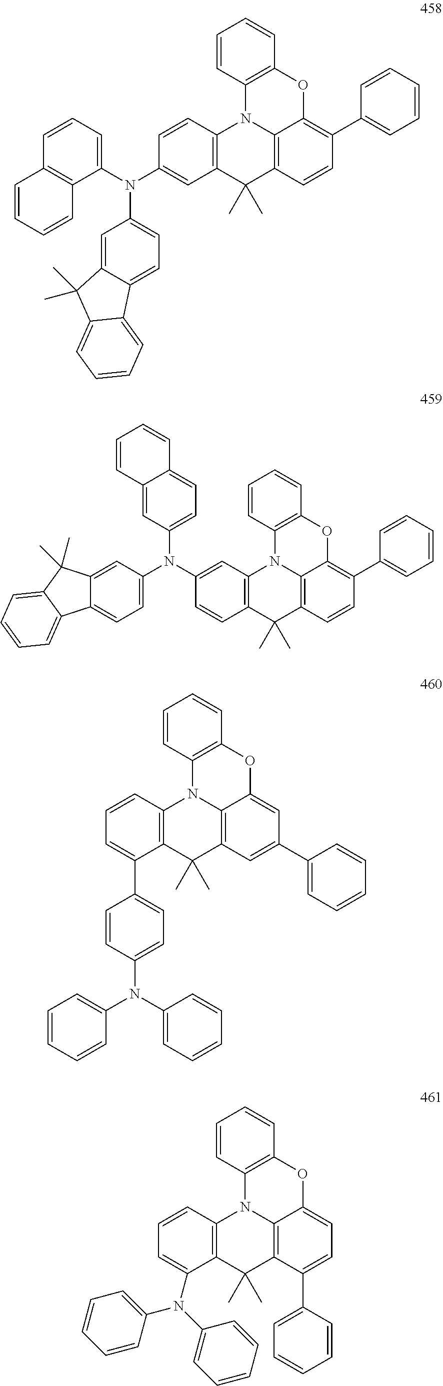 [SMILES: CC1(C)c2ccccc2-c2ccc(N(c3ccc4c(c3)C(C)(C)c3ccc(-c5ccccc5)c5c3N4c3ccccc3O5)c3cccc4ccccc34)cc21 ; CC1(C)c2ccccc2-c2ccc(N(c3ccc4c(c3)N3c5ccccc5Oc5c(-c6ccccc6)ccc(c53)C4(C)C)c3ccc4ccccc4c3)cc21 ; CC1(C)c2cc(-c3ccccc3)cc3c2N(c2ccccc2O3)c2cccc(-c3ccc(N(c4ccccc4)c4ccccc4)cc3)c21 ; CC1(C)c2c(N(c3ccccc3)c3ccccc3)cccc2N2c3ccccc3Oc3ccc(-c4ccccc4)c1c32]

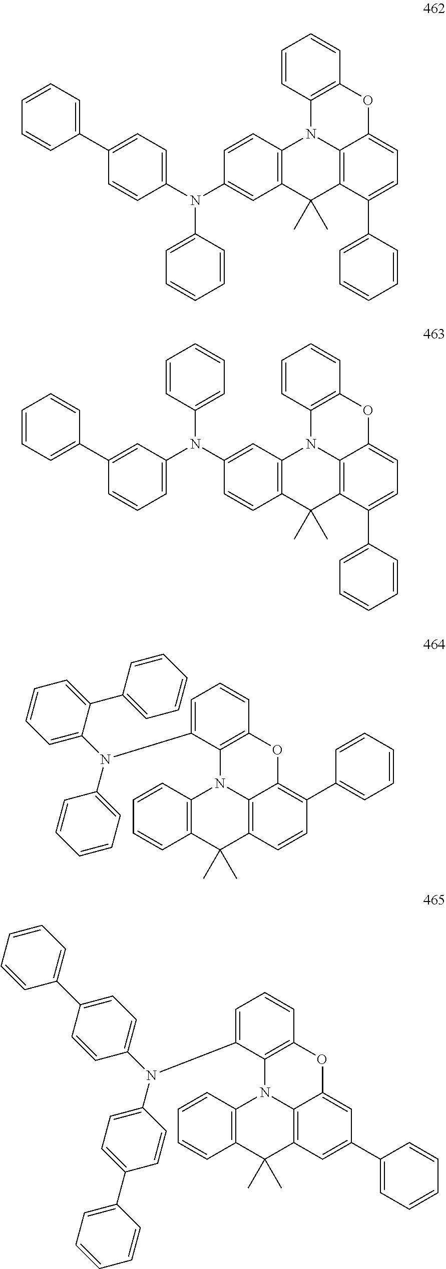

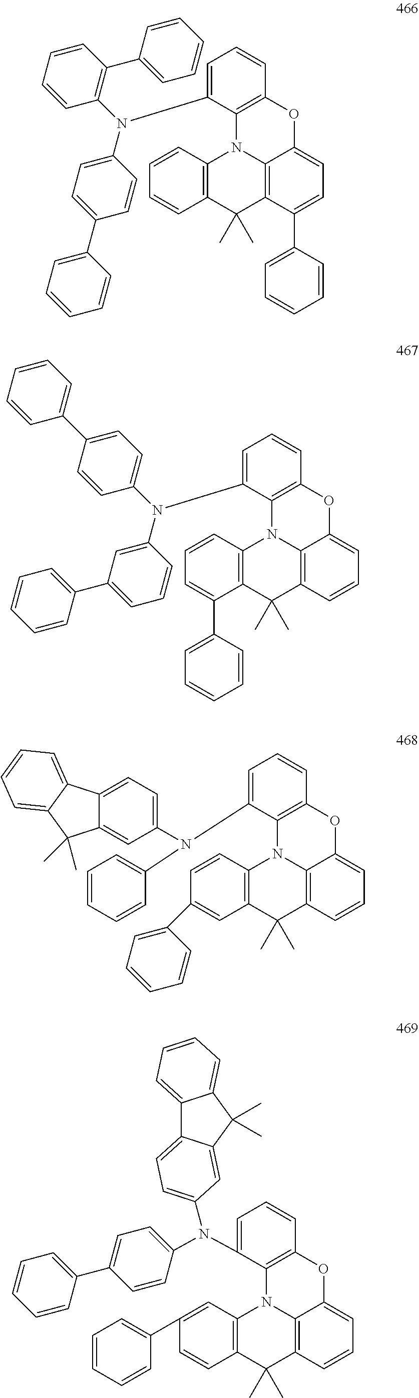 [SMILES: CC1(C)c2ccccc2N2c3c(cccc3N(c3ccc(-c4ccccc4)cc3)c3ccccc3-c3ccccc3)Oc3ccc(-c4ccccc4)c1c32 ; CC1(C)c2cccc3c2N(c2cccc(-c4ccccc4)c21)c1c(cccc1N(c1ccc(-c2ccccc2)cc1)c1cccc(-c2ccccc2)c1)O3 ; CC1(C)c2ccccc2-c2ccc(N(c3ccccc3)c3cccc4c3N3c5ccc(-c6ccccc6)cc5C(C)(C)c5cccc(c53)O4)cc21 ; CC1(C)c2ccccc2-c2ccc(N(c3ccc(-c4ccccc4)cc3)c3cccc4c3N3c5cc(-c6ccccc6)ccc5C(C)(C)c5cccc(c53)O4)cc21]

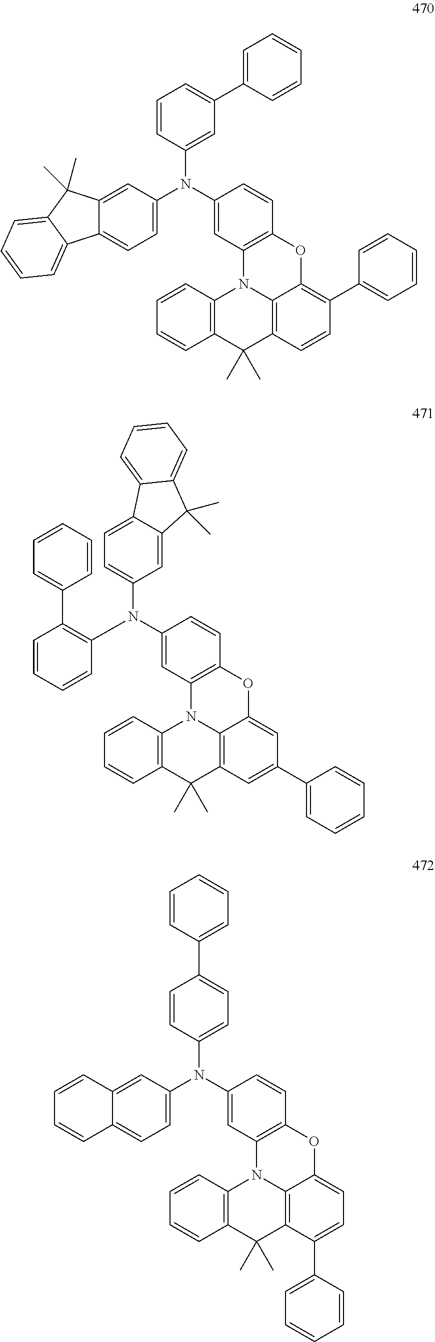 [SMILES: CC1(C)c2ccccc2-c2ccc(N(c3cccc(-c4ccccc4)c3)c3ccc4c(c3)N3c5ccccc5C(C)(C)c5ccc(-c6ccccc6)c(c53)O4)cc21 ; CC1(C)c2ccccc2-c2ccc(N(c3ccc4c(c3)N3c5ccccc5C(C)(C)c5cc(-c6ccccc6)cc(c53)O4)c3ccccc3-c3ccccc3)cc21 ; CC1(C)c2ccccc2N2c3cc(N(c4ccc(-c5ccccc5)cc4)c4ccc5ccccc5c4)ccc3Oc3ccc(-c4ccccc4)c1c32]

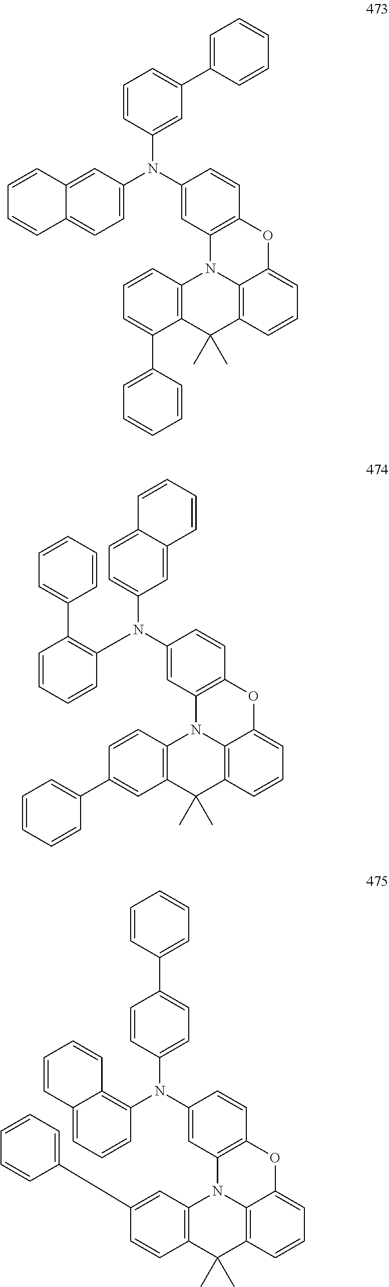

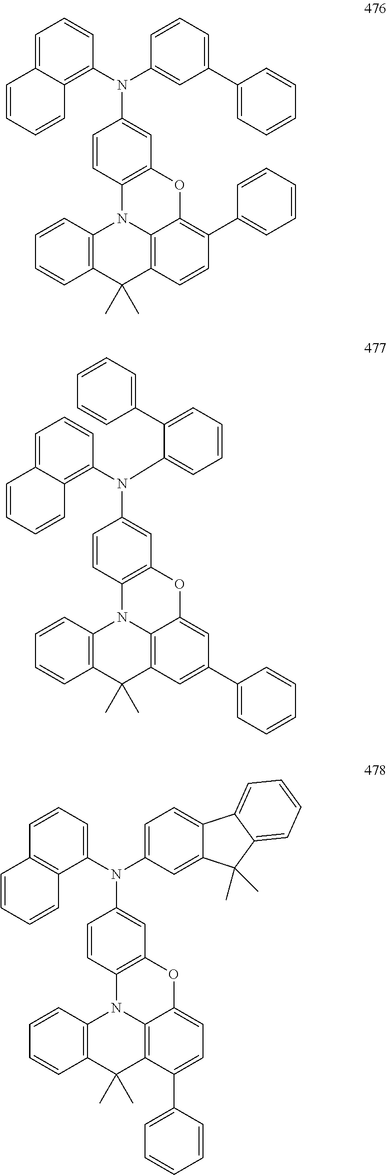

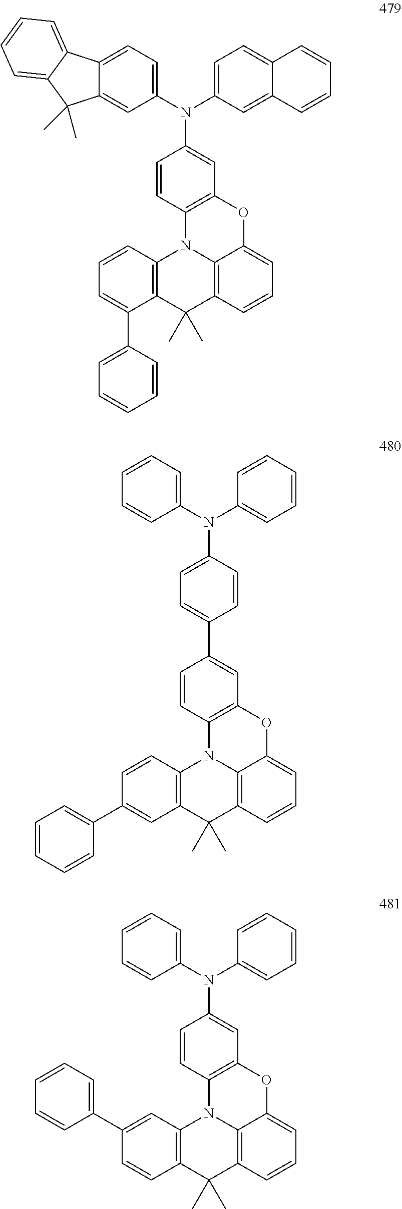 [SMILES: CC1(C)c2ccccc2-c2ccc(N(c3ccc4c(c3)Oc3cccc5c3N4c3cccc(-c4ccccc4)c3C5(C)C)c3ccc4ccccc4c3)cc21 ; CC1(C)c2cc(-c3ccccc3)ccc2N2c3ccc(-c4ccc(N(c5ccccc5)c5ccccc5)cc4)cc3Oc3cccc1c32 ; CC1(C)c2ccc(-c3ccccc3)cc2N2c3ccc(N(c4ccccc4)c4ccccc4)cc3Oc3cccc1c32]

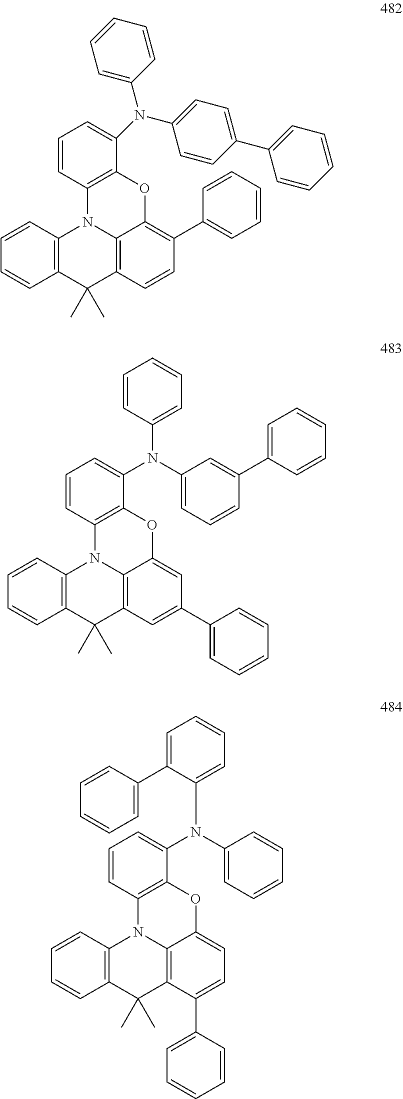 [SMILES: CC1(C)c2ccccc2N2c3cccc(N(c4ccccc4)c4ccc(-c5ccccc5)cc4)c3Oc3c(-c4ccccc4)ccc1c32 ; CC1(C)c2ccccc2N2c3cccc(N(c4ccccc4)c4cccc(-c5ccccc5)c4)c3Oc3cc(-c4ccccc4)cc1c32 ; CC1(C)c2ccccc2N2c3cccc(N(c4ccccc4)c4ccccc4-c4ccccc4)c3Oc3ccc(-c4ccccc4)c1c32]

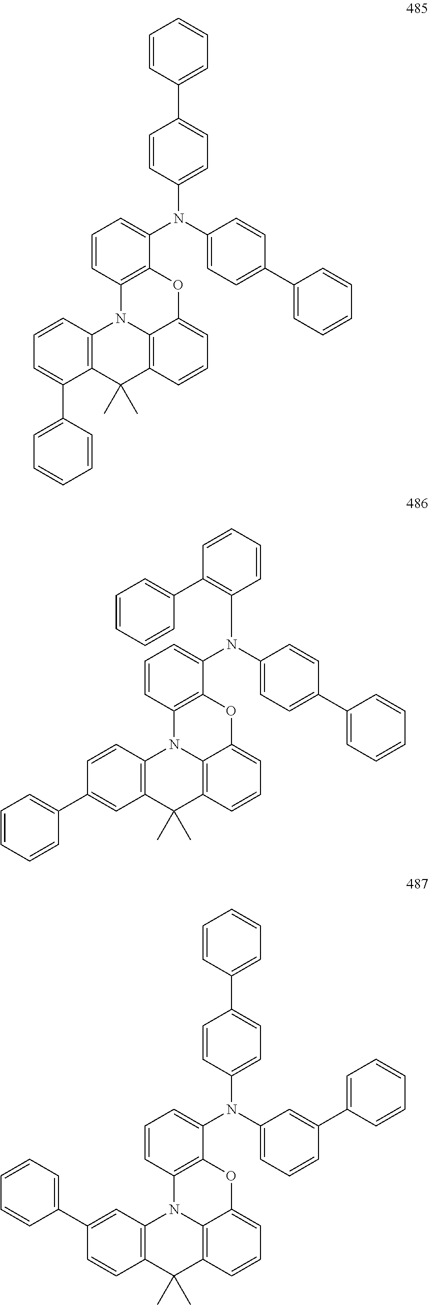 [SMILES: CC1(C)c2cccc3c2N(c2cccc(N(c4ccc(-c5ccccc5)cc4)c4ccc(-c5ccccc5)cc4)c2O3)c2cccc(-c3ccccc3)c21 ; CC1(C)c2cc(-c3ccccc3)ccc2N2c3cccc(N(c4ccc(-c5ccccc5)cc4)c4ccccc4-c4ccccc4)c3Oc3cccc1c32 ; CC1(C)c2ccc(-c3ccccc3)cc2N2c3cccc(N(c4ccc(-c5ccccc5)cc4)c4cccc(-c5ccccc5)c4)c3Oc3cccc1c32]

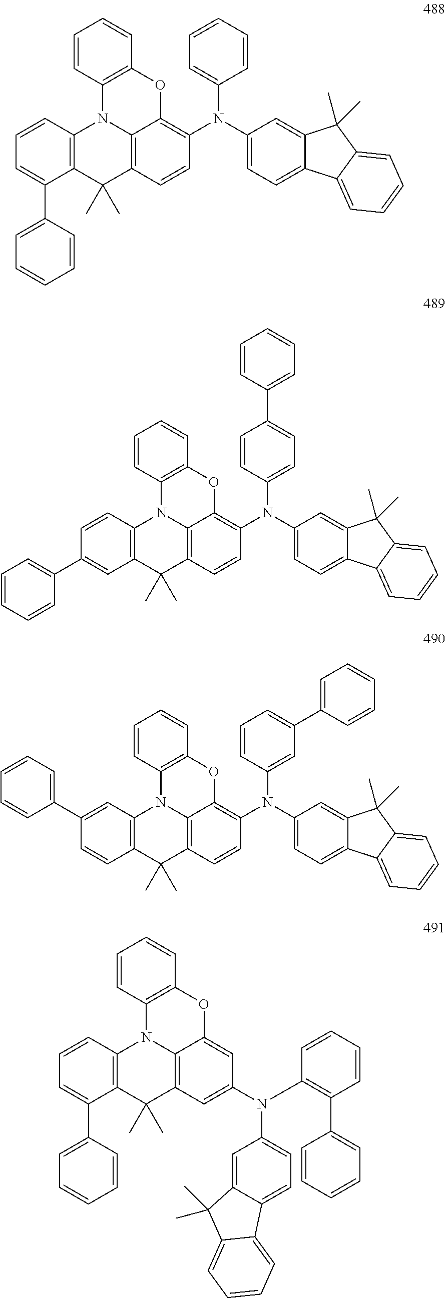 [SMILES: CC1(C)c2ccccc2-c2ccc(N(c3ccccc3)c3ccc4c5c3Oc3ccccc3N5c3cccc(-c5ccccc5)c3C4(C)C)cc21 ; CC1(C)c2ccccc2-c2ccc(N(c3ccc(-c4ccccc4)cc3)c3ccc4c5c3Oc3ccccc3N5c3ccc(-c5ccccc5)cc3C4(C)C)cc21 ; CC1(C)c2ccccc2-c2ccc(N(c3cccc(-c4ccccc4)c3)c3ccc4c5c3Oc3ccccc3N5c3cc(-c5ccccc5)ccc3C4(C)C)cc21 ; CC1(C)c2ccccc2-c2ccc(N(c3cc4c5c(c3)C(C)(C)c3c(-c6ccccc6)cccc3N5c3ccccc3O4)c3ccccc3-c3ccccc3)cc21]

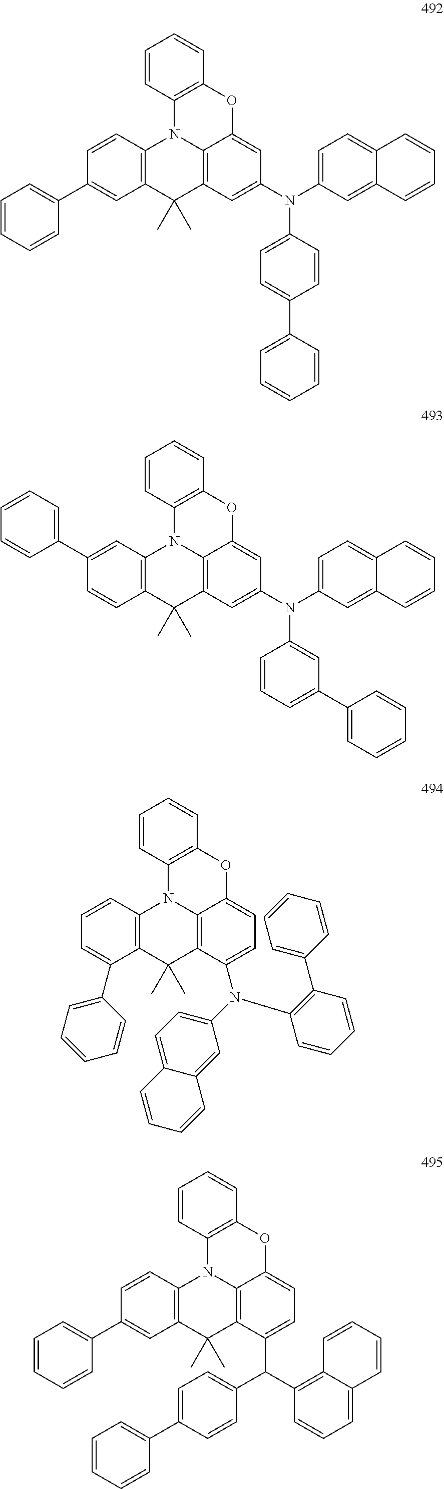 [SMILES: CC1(C)c2cc(-c3ccccc3)ccc2N2c3ccccc3Oc3cc(N(c4ccc(-c5ccccc5)cc4)c4ccc5ccccc5c4)cc1c32 ; CC1(C)c2ccc(-c3ccccc3)cc2N2c3ccccc3Oc3cc(N(c4cccc(-c5ccccc5)c4)c4ccc5ccccc5c4)cc1c32 ; CC1(C)c2c(-c3ccccc3)cccc2N2c3ccccc3Oc3ccc(N(c4ccc5ccccc5c4)c4ccccc4-c4ccccc4)c1c32 ; CC1(C)c2cc(-c3ccccc3)ccc2N2c3ccccc3Oc3ccc(C(c4ccc(-c5ccccc5)cc4)c4cccc5ccccc45)c1c32]

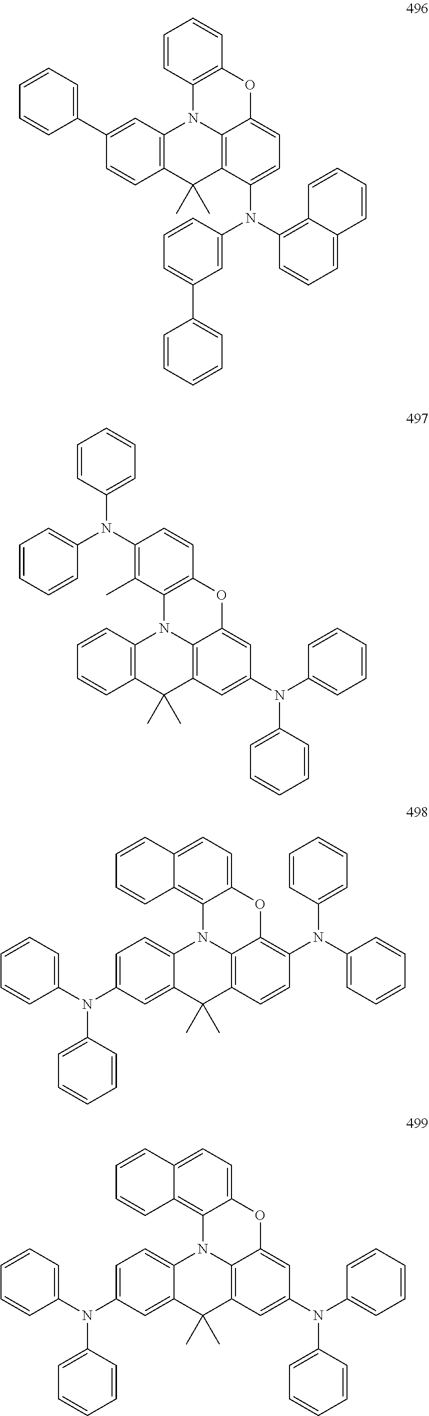 [SMILES: CC1(C)c2ccc(-c3ccccc3)cc2N2c3ccccc3Oc3ccc(N(c4cccc(-c5ccccc5)c4)c4cccc5ccccc45)c1c32 ; Cc1c(N(c2ccccc2)c2ccccc2)ccc2c1N1c3ccccc3C(C)(C)c3cc(N(c4ccccc4)c4ccccc4)cc(c31)O2 ; CC1(C)c2cc(N(c3ccccc3)c3ccccc3)ccc2N2c3c1ccc(N(c1ccccc1)c1ccccc1)c3Oc1ccc3ccccc3c12 ; CC1(C)c2cc(N(c3ccccc3)c3ccccc3)ccc2N2c3c(cc(N(c4ccccc4)c4ccccc4)cc31)Oc1ccc3ccccc3c12]

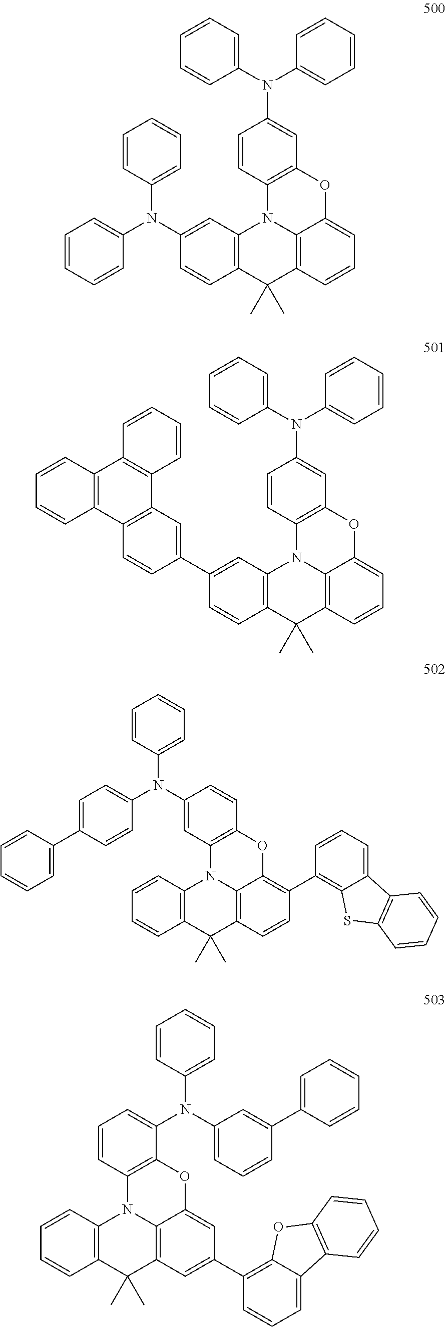

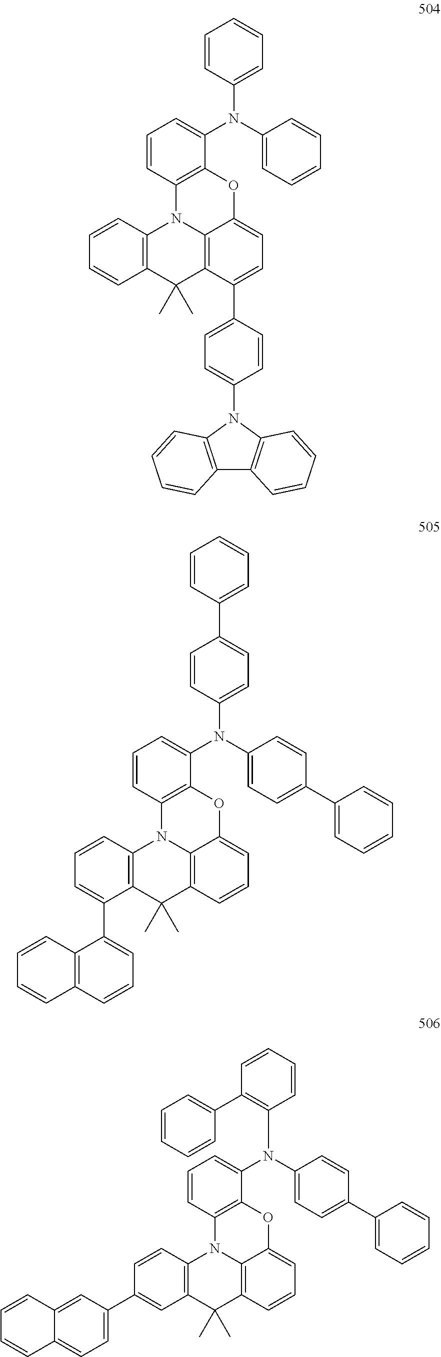 [SMILES: CC1(C)c2ccccc2N2c3cccc(N(c4ccccc4)c4ccccc4)c3Oc3ccc(-c4ccc(-n5c6ccccc6c6ccccc65)cc4)c1c32 ; CC1(C)c2cccc3c2N(c2cccc(N(c4ccc(-c5ccccc5)cc4)c4ccc(-c5ccccc5)cc4)c2O3)c2cccc(-c3cccc4ccccc34)c21 ; CC1(C)c2cc(-c3ccc4ccccc4c3)ccc2N2c3cccc(N(c4ccc(-c5ccccc5)cc4)c4ccccc4-c4ccccc4)c3Oc3cccc1c32]

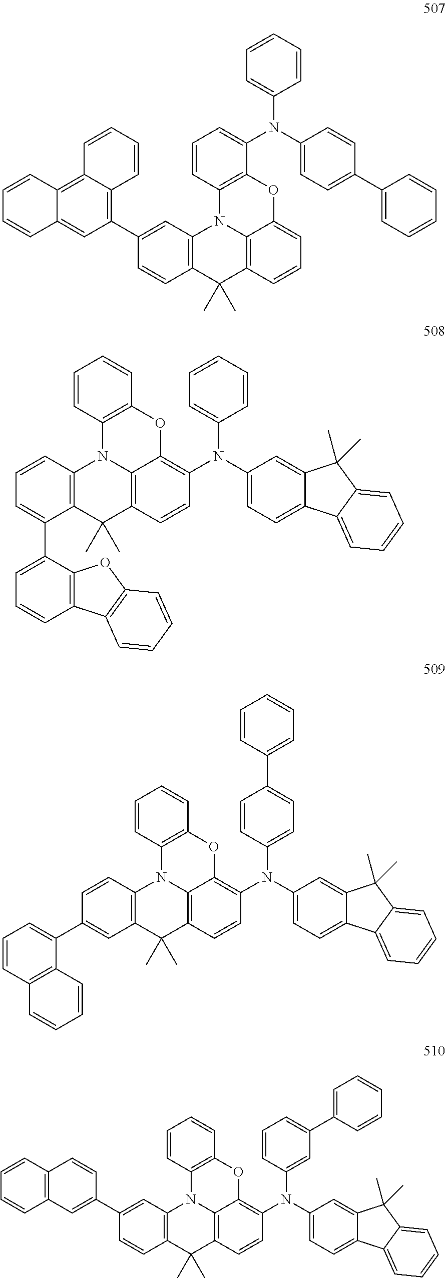 [SMILES: CC1(C)c2ccc(-c3cc4ccccc4c4ccccc34)cc2N2c3cccc(N(c4ccccc4)c4ccc(-c5ccccc5)cc4)c3Oc3cccc1c32 ; CC1(C)c2ccccc2-c2ccc(N(c3ccccc3)c3ccc4c5c3Oc3ccccc3N5c3cccc(-c5cccc6c5oc5ccccc56)c3C4(C)C)cc21 ; CC1(C)c2ccccc2-c2ccc(N(c3ccc(-c4ccccc4)cc3)c3ccc4c5c3Oc3ccccc3N5c3ccc(-c5cccc6ccccc56)cc3C4(C)C)cc21 ; CC1(C)c2ccccc2-c2ccc(N(c3cccc(-c4ccccc4)c3)c3ccc4c5c3Oc3ccccc3N5c3cc(-c5ccc6ccccc6c5)ccc3C4(C)C)cc21]

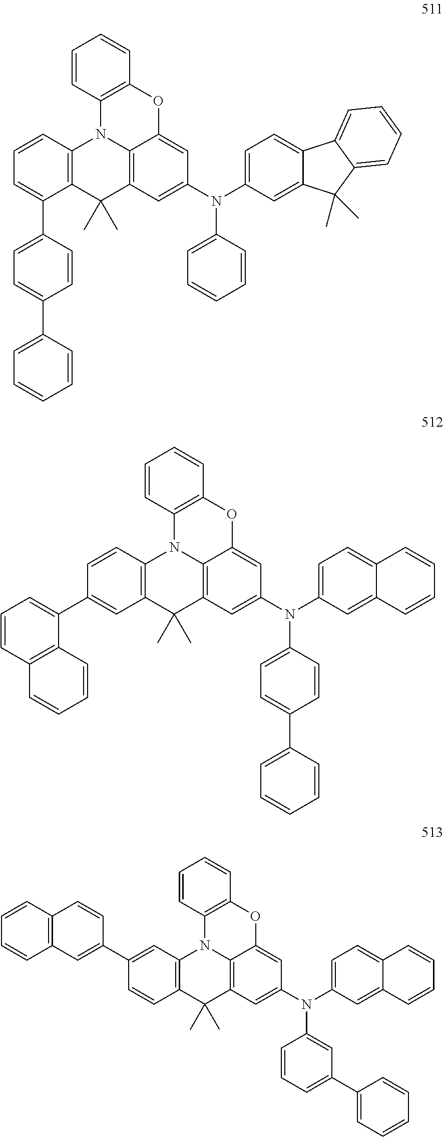 [SMILES: CC1(C)c2ccccc2-c2ccc(N(c3ccccc3)c3cc4c5c(c3)C(C)(C)c3c(-c6ccc(-c7ccccc7)cc6)cccc3N5c3ccccc3O4)cc21 ; CC1(C)c2cc(-c3cccc4ccccc34)ccc2N2c3ccccc3Oc3cc(N(c4ccc(-c5ccccc5)cc4)c4ccc5ccccc5c4)cc1c32 ; CC1(C)c2ccc(-c3ccc4ccccc4c3)cc2N2c3ccccc3Oc3cc(N(c4cccc(-c5ccccc5)c4)c4ccc5ccccc5c4)cc1c32]

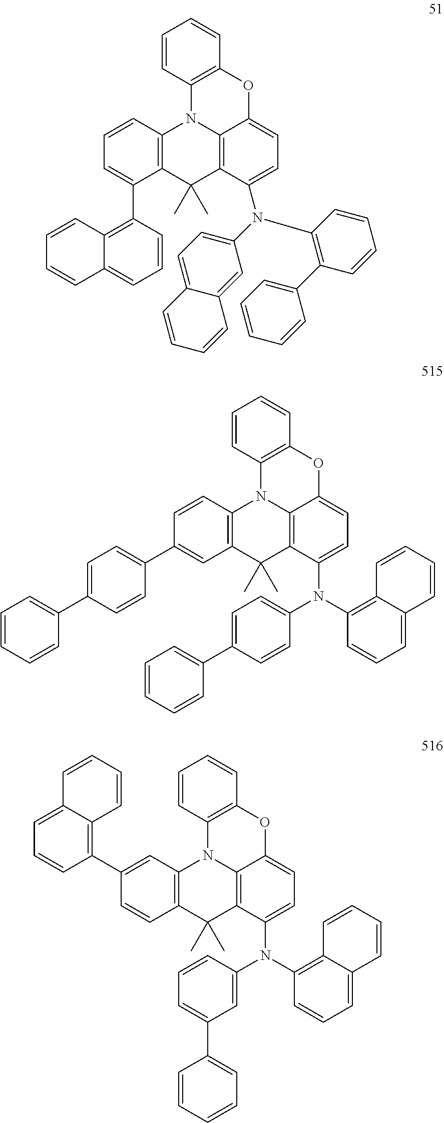 [SMILES: CC1(C)c2c(-c3cccc4ccccc34)cccc2N2c3ccccc3Oc3ccc(N(c4ccc5ccccc5c4)c4ccccc4-c4ccccc4)c1c32 ; CC1(C)c2cc(-c3ccc(-c4ccccc4)cc3)ccc2N2c3ccccc3Oc3ccc(N(c4ccc(-c5ccccc5)cc4)c4cccc5ccccc45)c1c32 ; CC1(C)c2ccc(-c3cccc4ccccc34)cc2N2c3ccccc3Oc3ccc(N(c4cccc(-c5ccccc5)c4)c4cccc5ccccc45)c1c32]

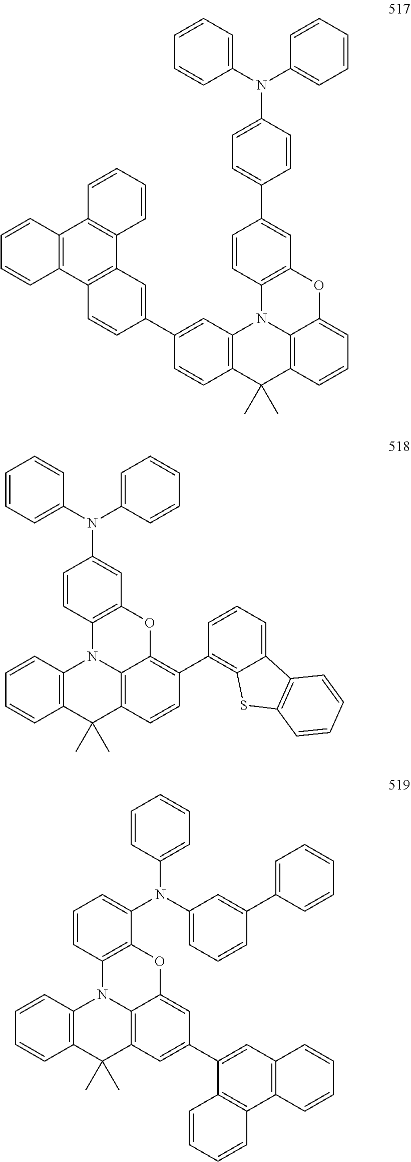 [SMILES: CC1(C)c2ccc(-c3ccc4c5ccccc5c5ccccc5c4c3)cc2N2c3ccc(-c4ccc(N(c5ccccc5)c5ccccc5)cc4)cc3Oc3cccc1c32 ; CC1(C)c2ccccc2N2c3ccc(N(c4ccccc4)c4ccccc4)cc3Oc3c(-c4cccc5c4sc4ccccc45)ccc1c32 ; CC1(C)c2ccccc2N2c3cccc(N(c4ccccc4)c4cccc(-c5ccccc5)c4)c3Oc3cc(-c4cc5ccccc5c5ccccc45)cc1c32]

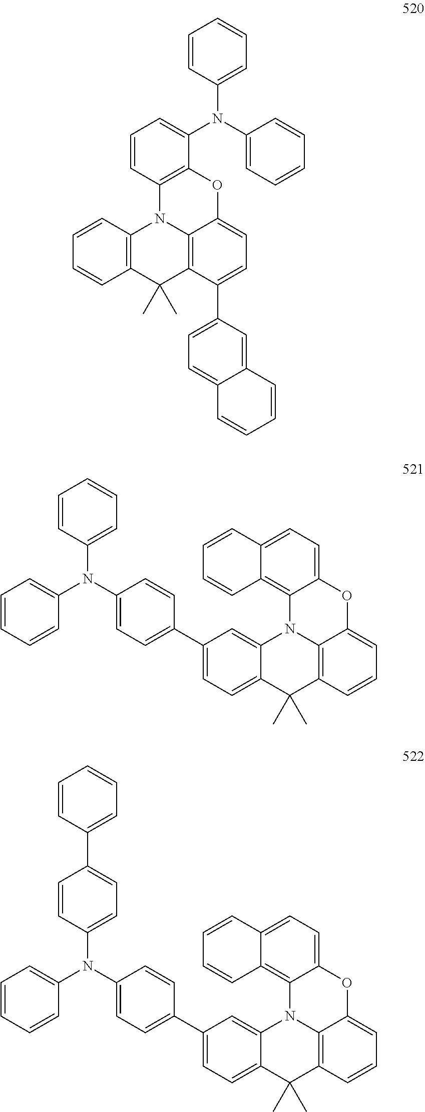 [SMILES: CC1(C)c2ccccc2N2c3cccc(N(c4ccccc4)c4ccccc4)c3Oc3ccc(-c4ccc5ccccc5c4)c1c32 ; CC1(C)c2ccc(-c3ccc(N(c4ccccc4)c4ccccc4)cc3)cc2N2c3c(cccc31)Oc1ccc3ccccc3c12 ; CC1(C)c2ccc(-c3ccc(N(c4ccccc4)c4ccc(-c5ccccc5)cc4)cc3)cc2N2c3c(cccc31)Oc1ccc3ccccc3c12]

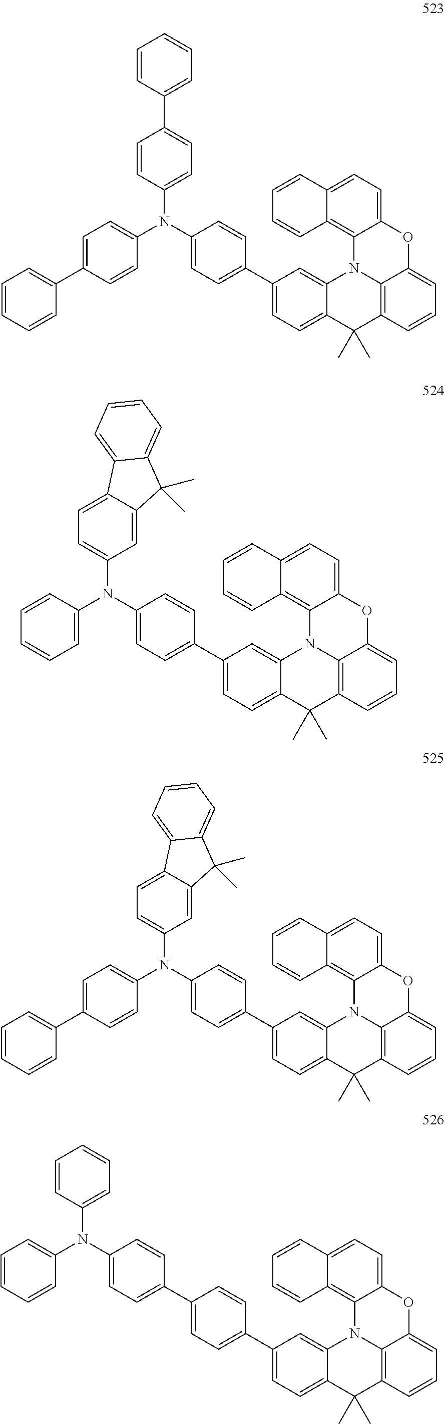 [SMILES: CC1(C)c2ccc(-c3ccc(N(c4ccc(-c5ccccc5)cc4)c4ccc(-c5ccccc5)cc4)cc3)cc2N2c3c(cccc31)Oc1ccc3ccccc3c12 ; CC1(C)c2ccccc2-c2ccc(N(c3ccccc3)c3ccc(-c4ccc5c(c4)N4c6c(cccc6C5(C)C)Oc5ccc6ccccc6c54)cc3)cc21 ; CC1(C)c2ccccc2-c2ccc(N(c3ccc(-c4ccccc4)cc3)c3ccc(-c4ccc5c(c4)N4c6c(cccc6C5(C)C)Oc5ccc6ccccc6c54)cc3)cc21 ; CC1(C)c2ccc(-c3ccc(-c4ccc(N(c5ccccc5)c5ccccc5)cc4)cc3)cc2N2c3c(cccc31)Oc1ccc3ccccc3c12]

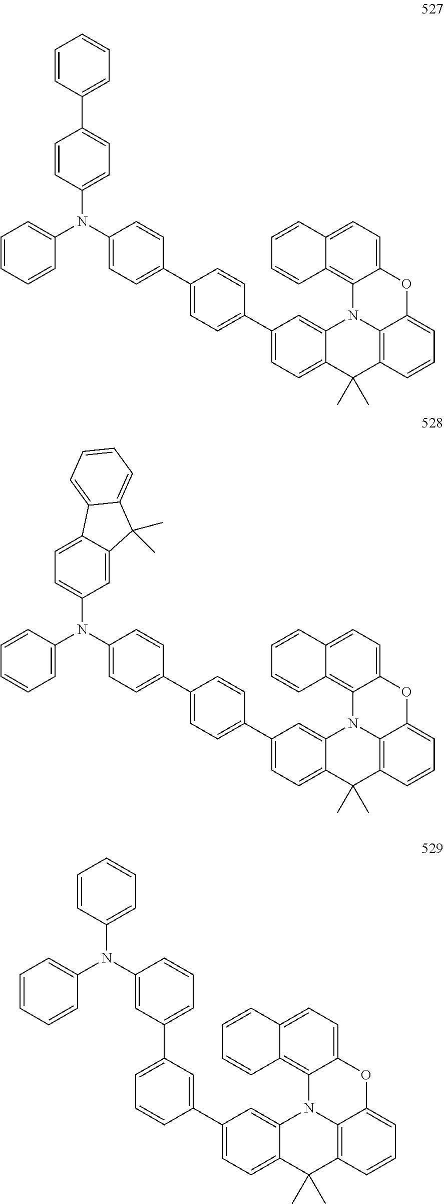 [SMILES: CC1(C)c2ccc(-c3ccc(-c4ccc(N(c5ccccc5)c5ccc(-c6ccccc6)cc5)cc4)cc3)cc2N2c3c(cccc31)Oc1ccc3ccccc3c12 ; CC1(C)c2ccccc2-c2ccc(N(c3ccccc3)c3ccc(-c4ccc(-c5ccc6c(c5)N5c7c(cccc7C6(C)C)Oc6ccc7ccccc7c65)cc4)cc3)cc21 ; CC1(C)c2ccc(-c3cccc(-c4cccc(N(c5ccccc5)c5ccccc5)c4)c3)cc2N2c3c(cccc31)Oc1ccc3ccccc3c12]

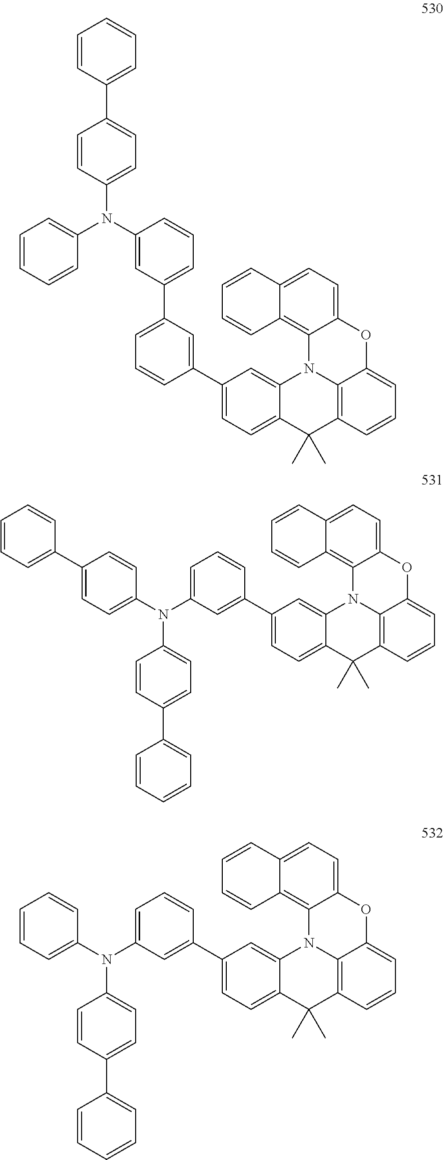

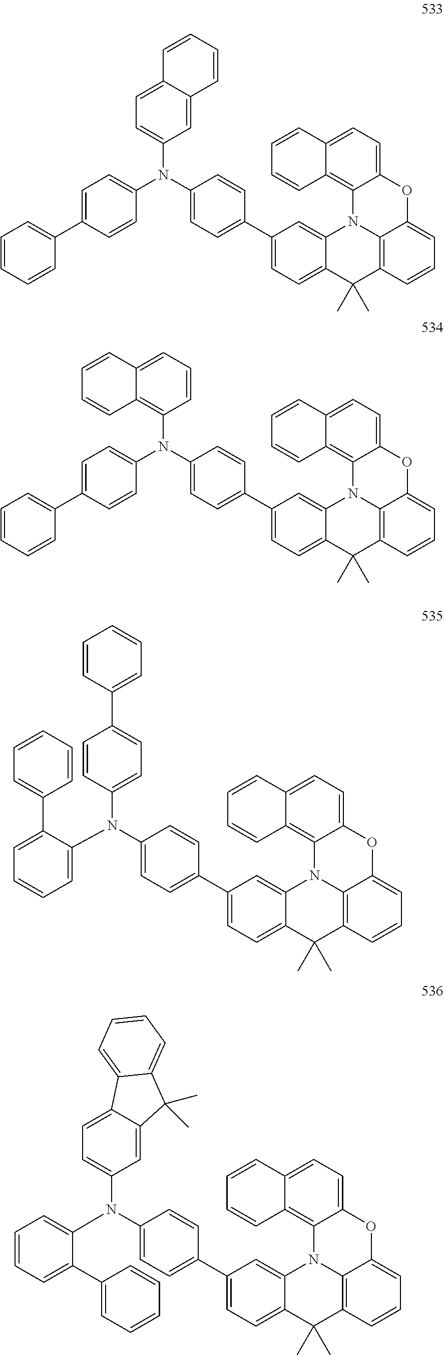 [SMILES: CC1(C)c2ccc(-c3ccc(N(c4ccc(-c5ccccc5)cc4)c4ccc5ccccc5c4)cc3)cc2N2c3c(cccc31)Oc1ccc3ccccc3c12 ; CC1(C)c2ccc(-c3ccc(N(c4ccc(-c5ccccc5)cc4)c4cccc5ccccc45)cc3)cc2N2c3c(cccc31)Oc1ccc3ccccc3c12 ; CC1(C)c2ccc(-c3ccc(N(c4ccc(-c5ccccc5)cc4)c4ccccc4-c4ccccc4)cc3)cc2N2c3c(cccc31)Oc1ccc3ccccc3c12 ; CC1(C)c2ccccc2-c2ccc(N(c3ccc(-c4ccc5c(c4)N4c6c(cccc6C5(C)C)Oc5ccc6ccccc6c54)cc3)c3ccccc3-c3ccccc3)cc21]

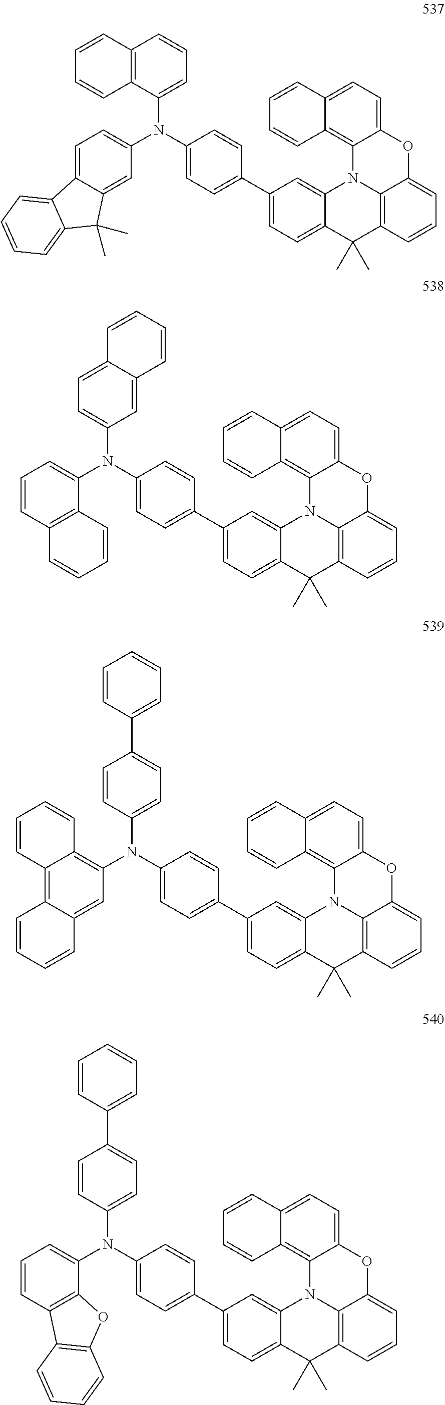 [SMILES: CC1(C)c2ccccc2-c2ccc(N(c3ccc(-c4ccc5c(c4)N4c6c(cccc6C5(C)C)Oc5ccc6ccccc6c54)cc3)c3cccc4ccccc34)cc21 ; CC1(C)c2ccc(-c3ccc(N(c4ccc5ccccc5c4)c4cccc5ccccc45)cc3)cc2N2c3c(cccc31)Oc1ccc3ccccc3c12 ; CC1(C)c2ccc(-c3ccc(N(c4ccc(-c5ccccc5)cc4)c4cc5ccccc5c5ccccc45)cc3)cc2N2c3c(cccc31)Oc1ccc3ccccc3c12 ; CC1(C)c2ccc(-c3ccc(N(c4ccc(-c5ccccc5)cc4)c4cccc5c4oc4ccccc45)cc3)cc2N2c3c(cccc31)Oc1ccc3ccccc3c12]

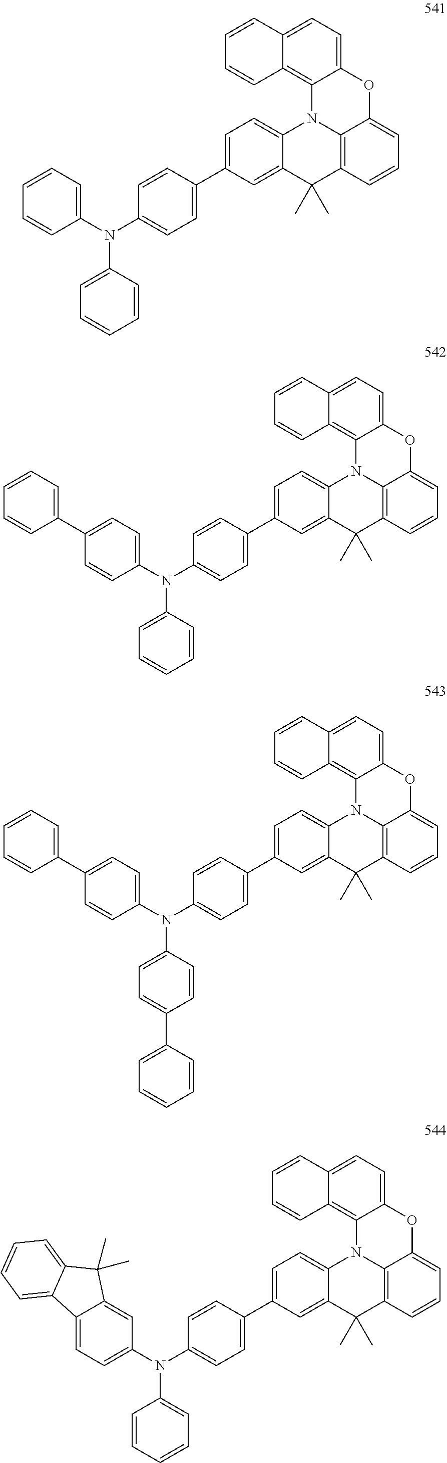 [SMILES: CC1(C)c2cc(-c3ccc(N(c4ccccc4)c4ccccc4)cc3)ccc2N2c3c(cccc31)Oc1ccc3ccccc3c12 ; CC1(C)c2cc(-c3ccc(N(c4ccccc4)c4ccc(-c5ccccc5)cc4)cc3)ccc2N2c3c(cccc31)Oc1ccc3ccccc3c12 ; CC1(C)c2cc(-c3ccc(N(c4ccc(-c5ccccc5)cc4)c4ccc(-c5ccccc5)cc4)cc3)ccc2N2c3c(cccc31)Oc1ccc3ccccc3c12 ; CC1(C)c2ccccc2-c2ccc(N(c3ccccc3)c3ccc(-c4ccc5c(c4)C(C)(C)c4cccc6c4N5c4c(ccc5ccccc45)O6)cc3)cc21]

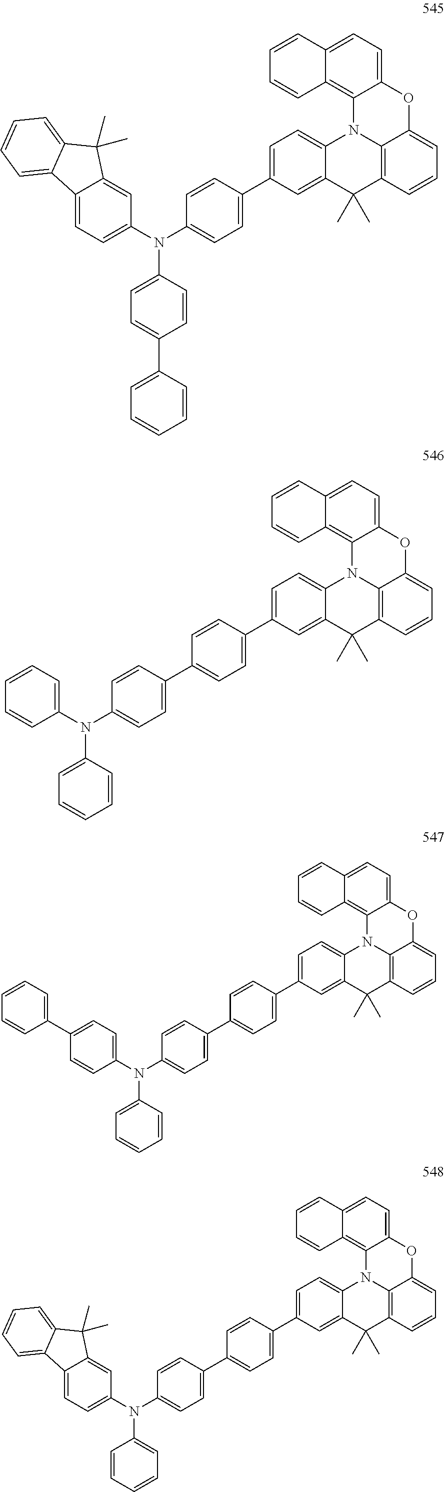 [SMILES: CC1(C)c2ccccc2-c2ccc(N(c3ccc(-c4ccccc4)cc3)c3ccc(-c4ccc5c(c4)C(C)(C)c4cccc6c4N5c4c(ccc5ccccc45)O6)cc3)cc21 ; CC1(C)c2cc(-c3ccc(-c4ccc(N(c5ccccc5)c5ccccc5)cc4)cc3)ccc2N2c3c(cccc31)Oc1ccc3ccccc3c12 ; CC1(C)c2cc(-c3ccc(-c4ccc(N(c5ccccc5)c5ccc(-c6ccccc6)cc5)cc4)cc3)ccc2N2c3c(cccc31)Oc1ccc3ccccc3c12 ; CC1(C)c2ccccc2-c2ccc(N(c3ccccc3)c3ccc(-c4ccc(-c5ccc6c(c5)C(C)(C)c5cccc7c5N6c5c(ccc6ccccc56)O7)cc4)cc3)cc21]

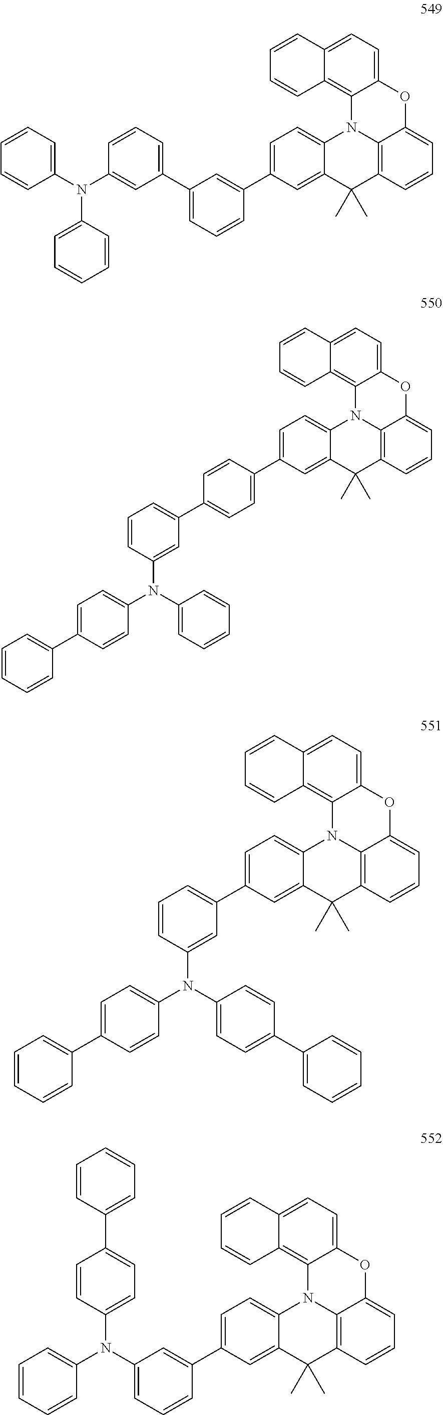 [SMILES: CC1(C)c2cc(-c3cccc(-c4cccc(N(c5ccccc5)c5ccccc5)c4)c3)ccc2N2c3c(cccc31)Oc1ccc3ccccc3c12 ; CC1(C)c2cc(-c3ccc(-c4cccc(N(c5ccccc5)c5ccc(-c6ccccc6)cc5)c4)cc3)ccc2N2c3c(cccc31)Oc1ccc3ccccc3c12 ; CC1(C)c2cc(-c3cccc(N(c4ccc(-c5ccccc5)cc4)c4ccc(-c5ccccc5)cc4)c3)ccc2N2c3c(cccc31)Oc1ccc3ccccc3c12 ; CC1(C)c2cc(-c3cccc(N(c4ccccc4)c4ccc(-c5ccccc5)cc4)c3)ccc2N2c3c(cccc31)Oc1ccc3ccccc3c12]

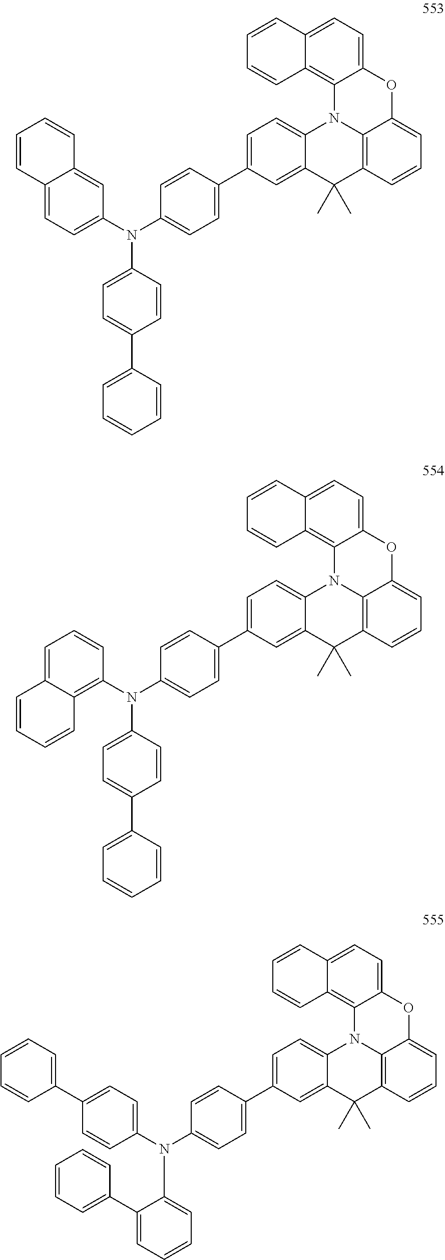 [SMILES: CC1(C)c2cc(-c3ccc(N(c4ccc(-c5ccccc5)cc4)c4ccc5ccccc5c4)cc3)ccc2N2c3c(cccc31)Oc1ccc3ccccc3c12 ; CC1(C)c2cc(-c3ccc(N(c4ccc(-c5ccccc5)cc4)c4cccc5ccccc45)cc3)ccc2N2c3c(cccc31)Oc1ccc3ccccc3c12 ; CC1(C)c2cc(-c3ccc(N(c4ccc(-c5ccccc5)cc4)c4ccccc4-c4ccccc4)cc3)ccc2N2c3c(cccc31)Oc1ccc3ccccc3c12]

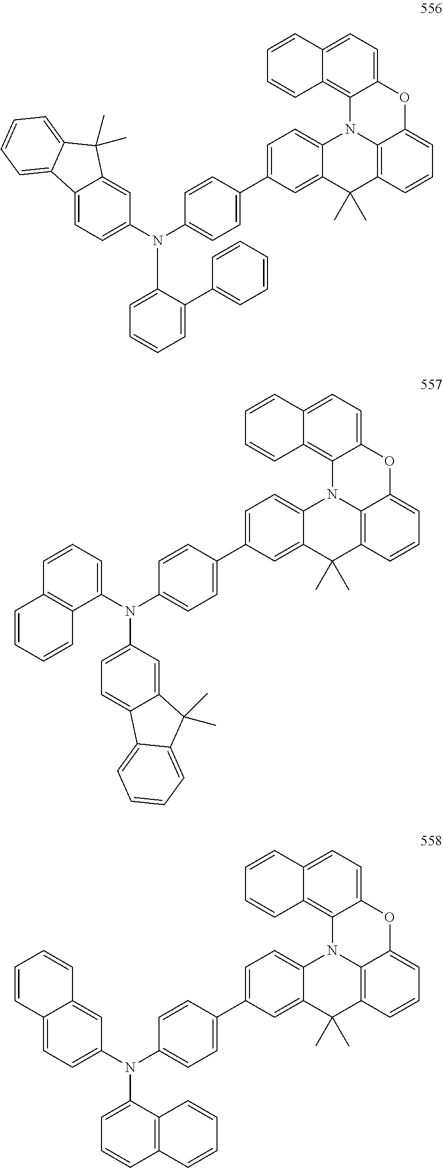 [SMILES: CC1(C)c2ccccc2-c2ccc(N(c3ccc(-c4ccc5c(c4)C(C)(C)c4cccc6c4N5c4c(ccc5ccccc45)O6)cc3)c3ccccc3-c3ccccc3)cc21 ; CC1(C)c2ccccc2-c2ccc(N(c3ccc(-c4ccc5c(c4)C(C)(C)c4cccc6c4N5c4c(ccc5ccccc45)O6)cc3)c3cccc4ccccc34)cc21 ; CC1(C)c2cc(-c3ccc(N(c4ccc5ccccc5c4)c4cccc5ccccc45)cc3)ccc2N2c3c(cccc31)Oc1ccc3ccccc3c12]

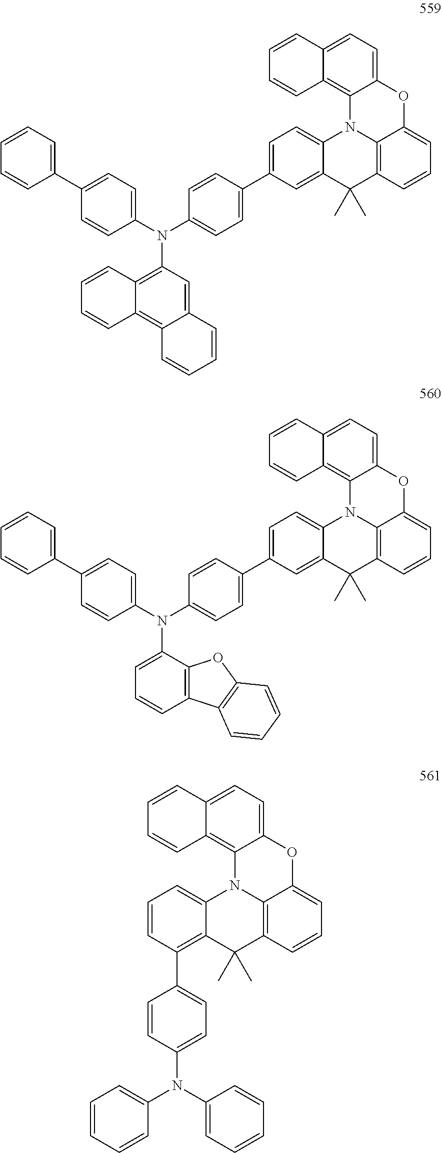 [SMILES: CC1(C)c2cc(-c3ccc(N(c4ccc(-c5ccccc5)cc4)c4cc5ccccc5c5ccccc45)cc3)ccc2N2c3c(cccc31)Oc1ccc3ccccc3c12 ; CC1(C)c2cc(-c3ccc(N(c4ccc(-c5ccccc5)cc4)c4cccc5c4oc4ccccc45)cc3)ccc2N2c3c(cccc31)Oc1ccc3ccccc3c12 ; CC1(C)c2cccc3c2N(c2cccc(-c4ccc(N(c5ccccc5)c5ccccc5)cc4)c21)c1c(ccc2ccccc12)O3]

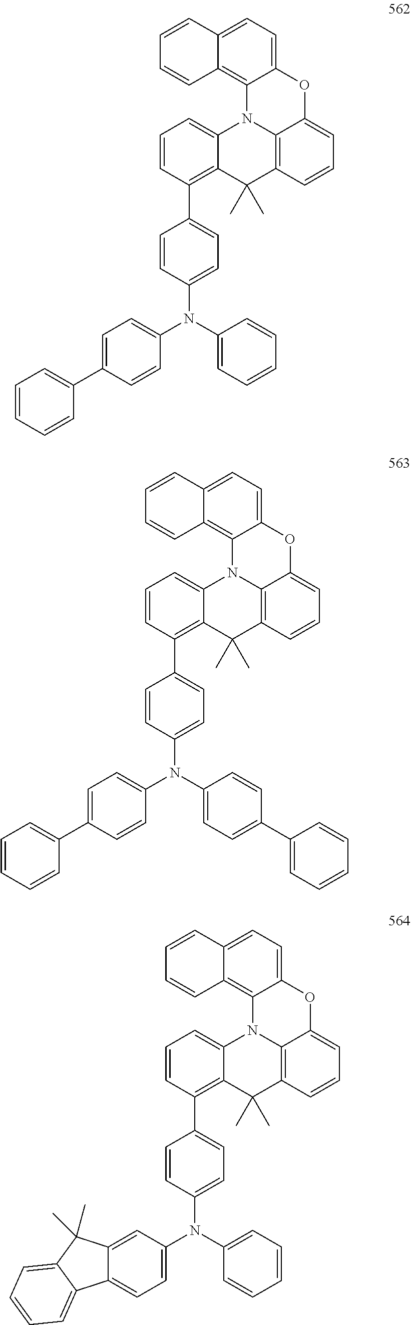 [SMILES: CC1(C)c2cccc3c2N(c2cccc(-c4ccc(N(c5ccccc5)c5ccc(-c6ccccc6)cc5)cc4)c21)c1c(ccc2ccccc12)O3 ; CC1(C)c2cccc3c2N(c2cccc(-c4ccc(N(c5ccc(-c6ccccc6)cc5)c5ccc(-c6ccccc6)cc5)cc4)c21)c1c(ccc2ccccc12)O3 ; CC1(C)c2ccccc2-c2ccc(N(c3ccccc3)c3ccc(-c4cccc5c4C(C)(C)c4cccc6c4N5c4c(ccc5ccccc45)O6)cc3)cc21]

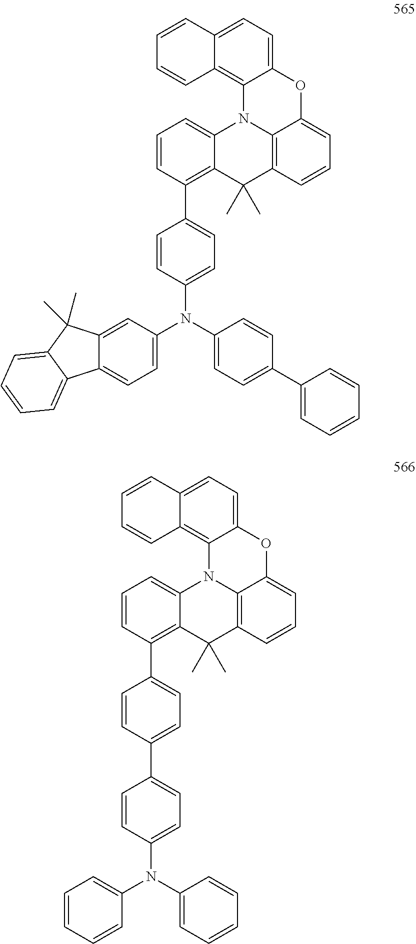 [SMILES: CC1(C)c2ccccc2-c2ccc(N(c3ccc(-c4ccccc4)cc3)c3ccc(-c4cccc5c4C(C)(C)c4cccc6c4N5c4c(ccc5ccccc45)O6)cc3)cc21 ; CC1(C)c2cccc3c2N(c2cccc(-c4ccc(-c5ccc(N(c6ccccc6)c6ccccc6)cc5)cc4)c21)c1c(ccc2ccccc12)O3]

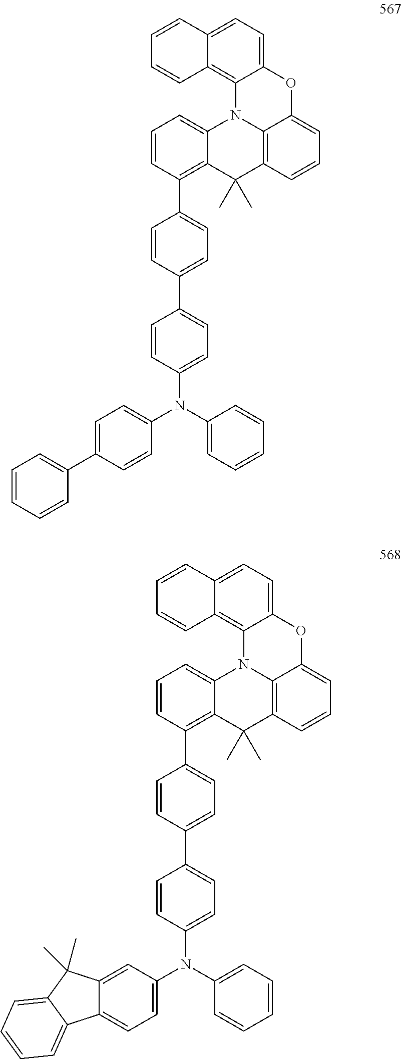 [SMILES: CC1(C)c2cccc3c2N(c2cccc(-c4ccc(-c5ccc(N(c6ccccc6)c6ccc(-c7ccccc7)cc6)cc5)cc4)c21)c1c(ccc2ccccc12)O3 ; CC1(C)c2ccccc2-c2ccc(N(c3ccccc3)c3ccc(-c4ccc(-c5cccc6c5C(C)(C)c5cccc7c5N6c5c(ccc6ccccc56)O7)cc4)cc3)cc21]

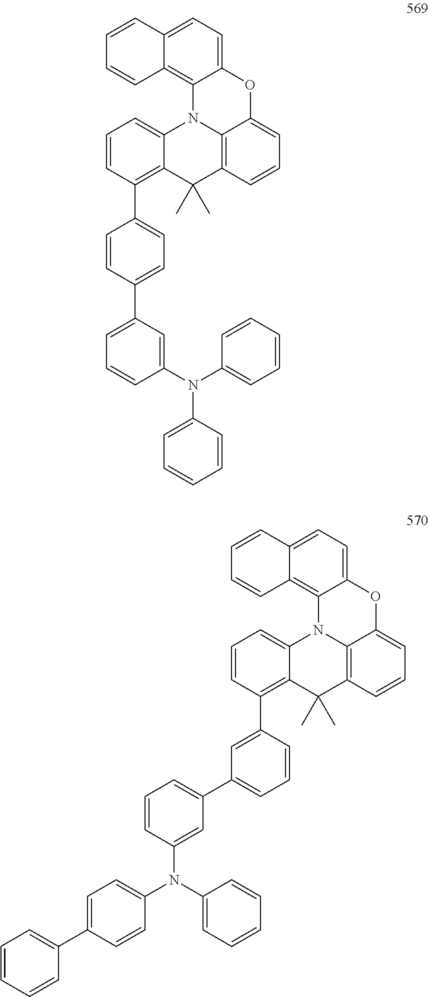 [SMILES: CC1(C)c2cccc3c2N(c2cccc(-c4ccc(-c5cccc(N(c6ccccc6)c6ccccc6)c5)cc4)c21)c1c(ccc2ccccc12)O3 ; CC1(C)c2cccc3c2N(c2cccc(-c4cccc(-c5cccc(N(c6ccccc6)c6ccc(-c7ccccc7)cc6)c5)c4)c21)c1c(ccc2ccccc12)O3]

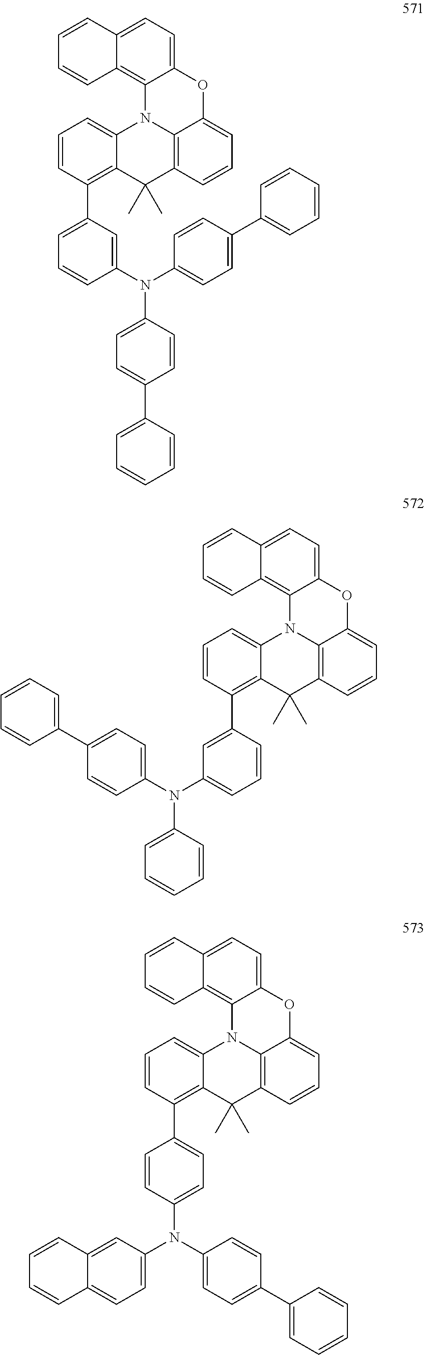

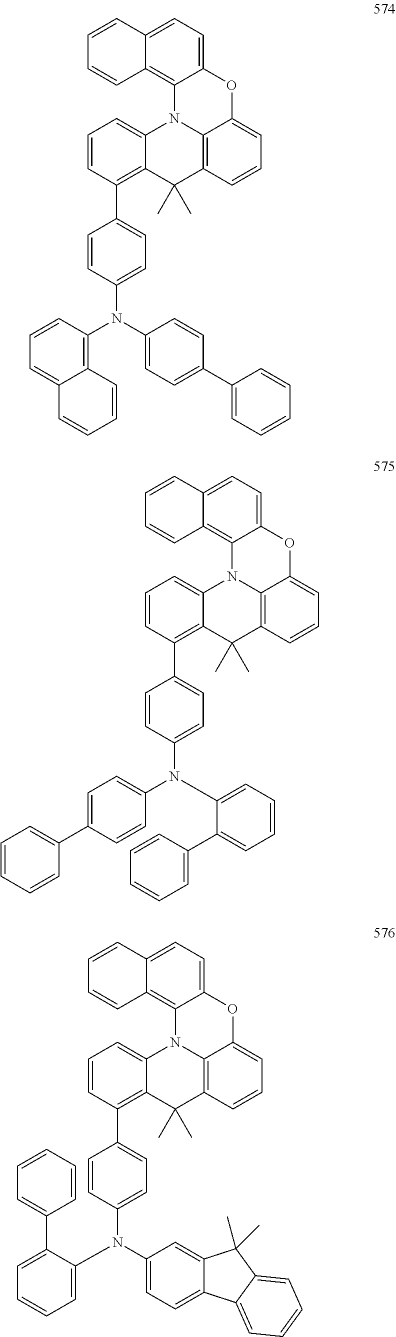

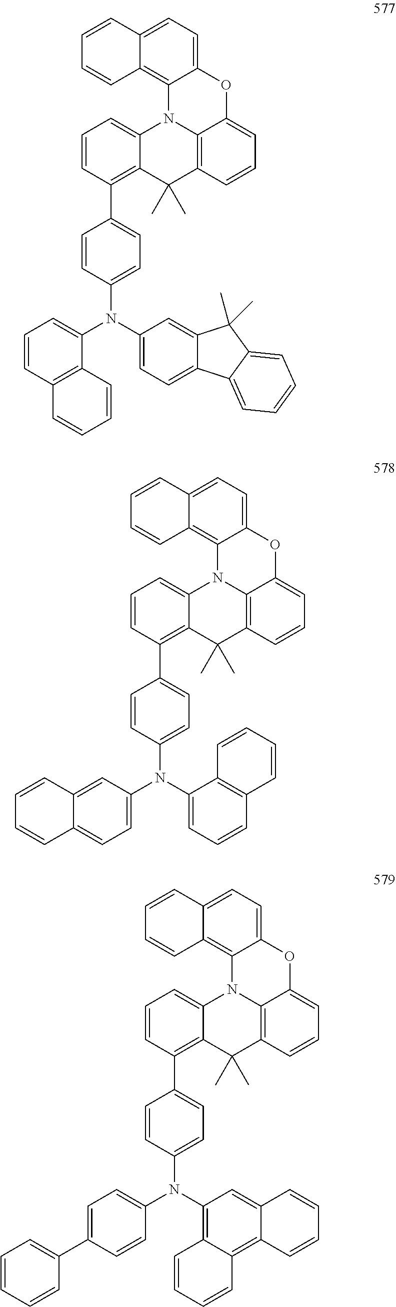 [SMILES: CC1(C)c2ccccc2-c2ccc(N(c3ccc(-c4cccc5c4C(C)(C)c4cccc6c4N5c4c(ccc5ccccc45)O6)cc3)c3cccc4ccccc34)cc21 ; CC1(C)c2cccc3c2N(c2cccc(-c4ccc(N(c5ccc6ccccc6c5)c5cccc6ccccc56)cc4)c21)c1c(ccc2ccccc12)O3 ; CC1(C)c2cccc3c2N(c2cccc(-c4ccc(N(c5ccc(-c6ccccc6)cc5)c5cc6ccccc6c6ccccc56)cc4)c21)c1c(ccc2ccccc12)O3]

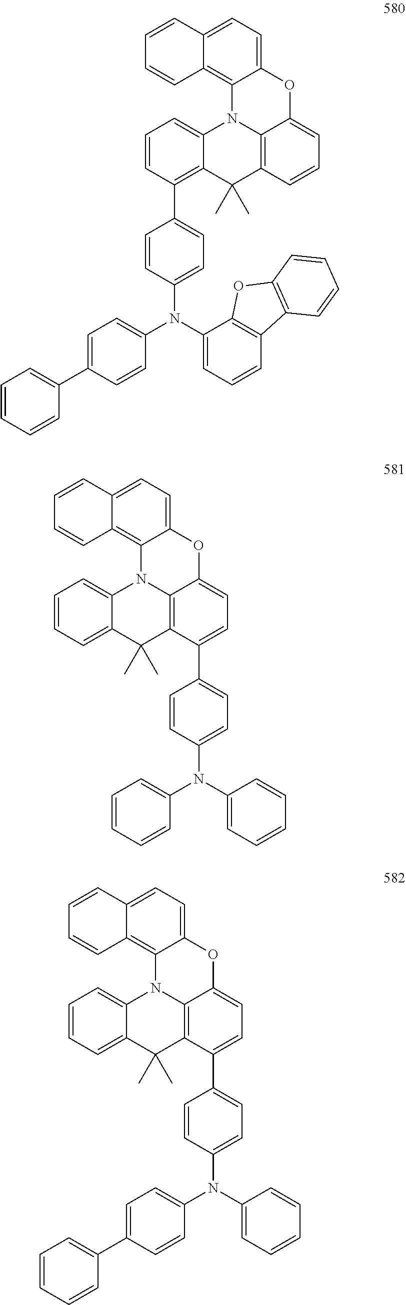

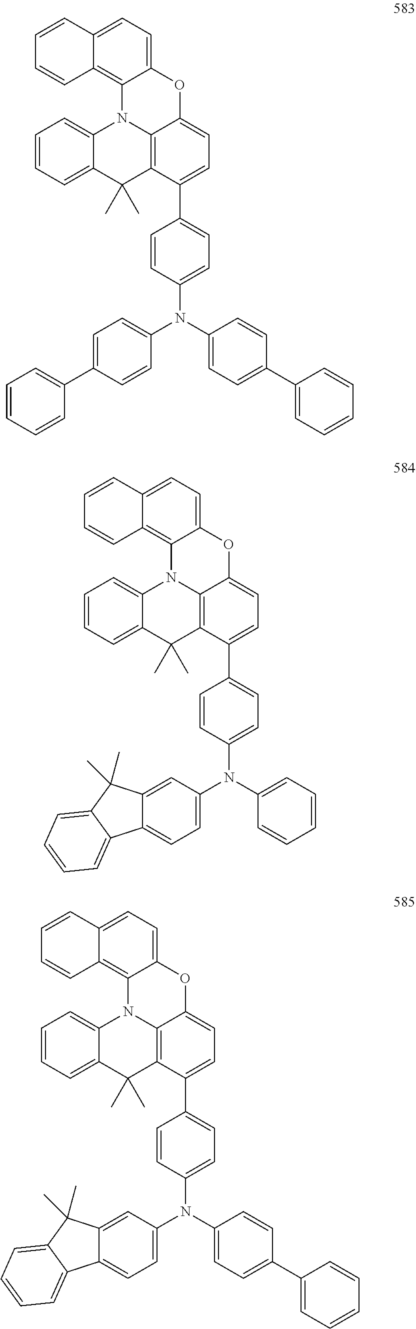

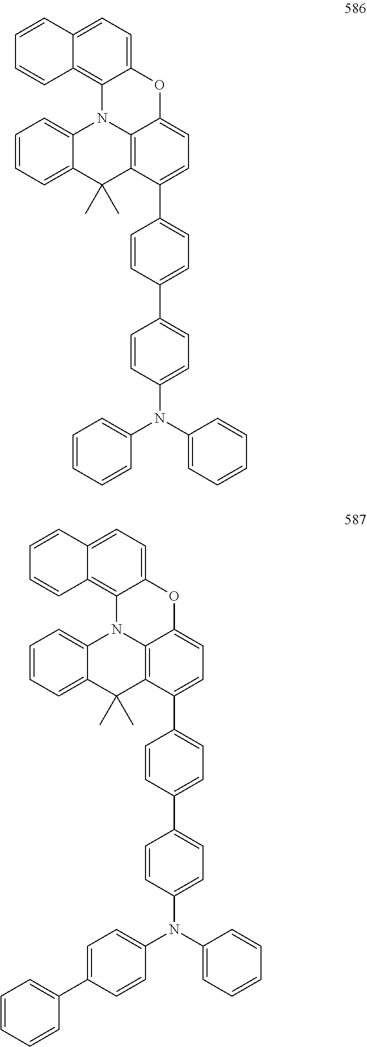

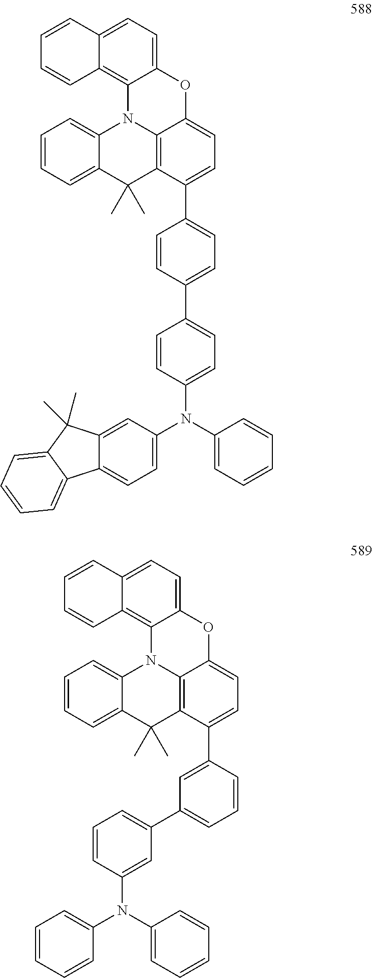 [SMILES: CC1(C)c2ccccc2-c2ccc(N(c3ccccc3)c3ccc(-c4ccc(-c5ccc6c7c5C(C)(C)c5ccccc5N7c5c(ccc7ccccc57)O6)cc4)cc3)cc21 ; CC1(C)c2ccccc2N2c3c(ccc(-c4cccc(-c5cccc(N(c6ccccc6)c6ccccc6)c5)c4)c31)Oc1ccc3ccccc3c12]

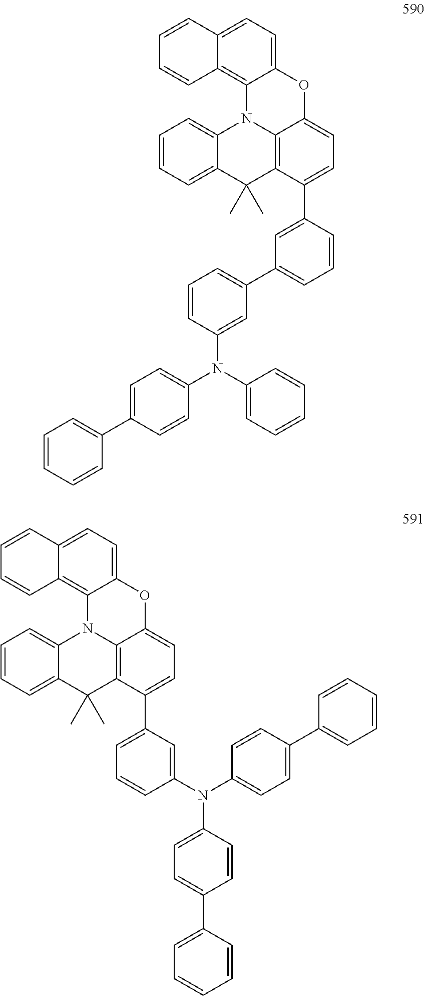

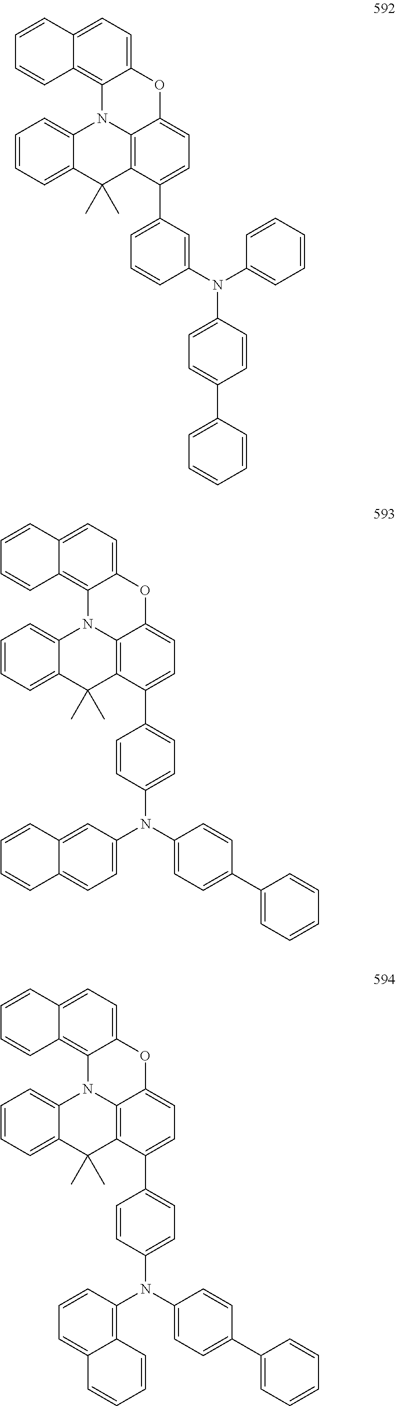 [SMILES: CC1(C)c2ccccc2N2c3c(ccc(-c4cccc(N(c5ccccc5)c5ccc(-c6ccccc6)cc5)c4)c31)Oc1ccc3ccccc3c12 ; CC1(C)c2ccccc2N2c3c(ccc(-c4ccc(N(c5ccc(-c6ccccc6)cc5)c5ccc6ccccc6c5)cc4)c31)Oc1ccc3ccccc3c12 ; CC1(C)c2ccccc2N2c3c(ccc(-c4ccc(N(c5ccc(-c6ccccc6)cc5)c5cccc6ccccc56)cc4)c31)Oc1ccc3ccccc3c12]

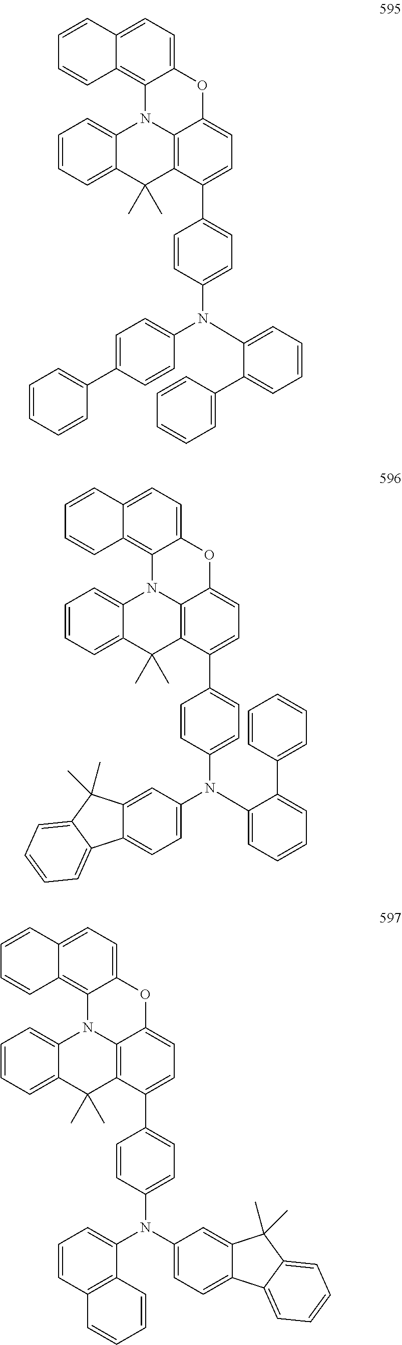 [SMILES: CC1(C)c2ccccc2N2c3c(ccc(-c4ccc(N(c5ccc(-c6ccccc6)cc5)c5ccccc5-c5ccccc5)cc4)c31)Oc1ccc3ccccc3c12 ; CC1(C)c2ccccc2-c2ccc(N(c3ccc(-c4ccc5c6c4C(C)(C)c4ccccc4N6c4c(ccc6ccccc46)O5)cc3)c3ccccc3-c3ccccc3)cc21 ; CC1(C)c2ccccc2-c2ccc(N(c3ccc(-c4ccc5c6c4C(C)(C)c4ccccc4N6c4c(ccc6ccccc46)O5)cc3)c3cccc4ccccc34)cc21]

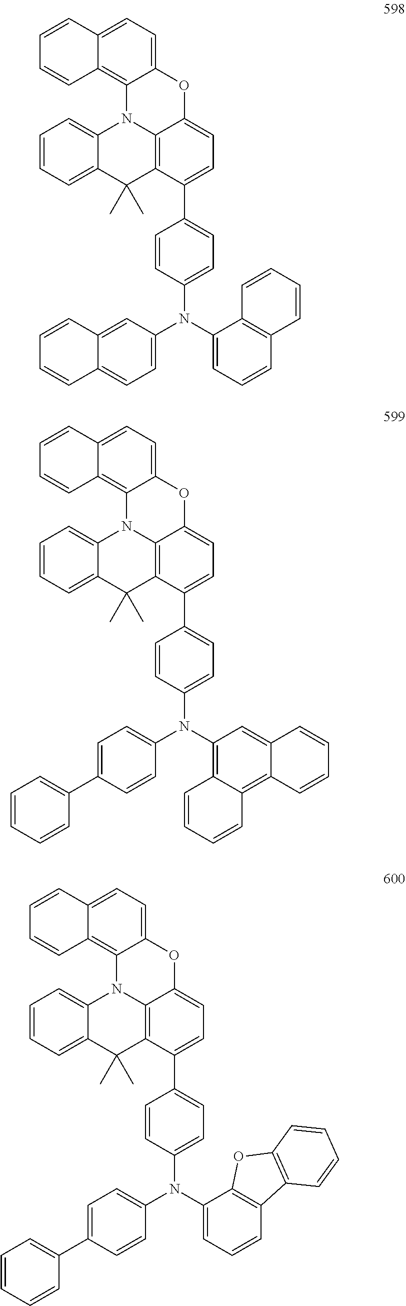

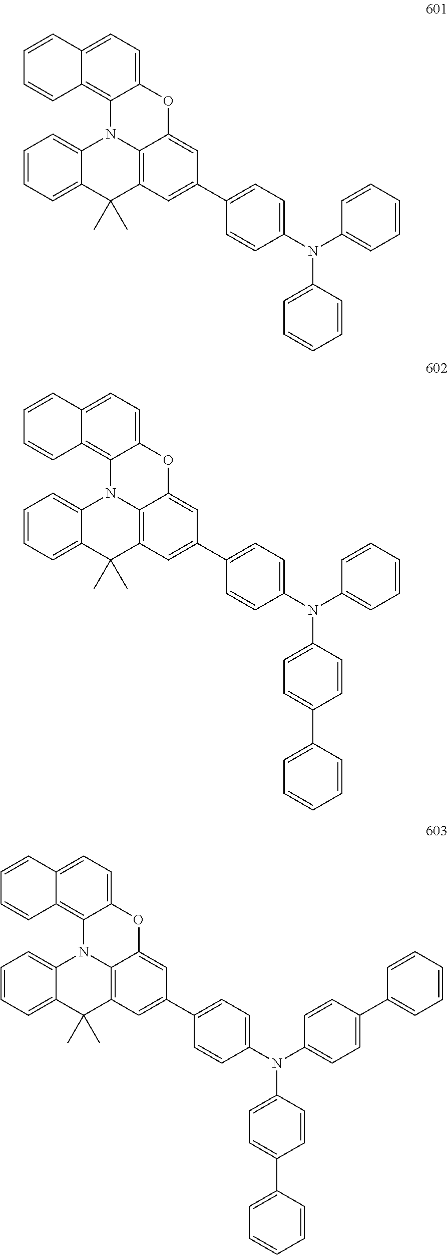 [SMILES: CC1(C)c2ccccc2N2c3c(cc(-c4ccc(N(c5ccccc5)c5ccccc5)cc4)cc31)Oc1ccc3ccccc3c12 ; CC1(C)c2ccccc2N2c3c(cc(-c4ccc(N(c5ccccc5)c5ccc(-c6ccccc6)cc5)cc4)cc31)Oc1ccc3ccccc3c12 ; CC1(C)c2ccccc2N2c3c(cc(-c4ccc(N(c5ccc(-c6ccccc6)cc5)c5ccc(-c6ccccc6)cc5)cc4)cc31)Oc1ccc3ccccc3c12]

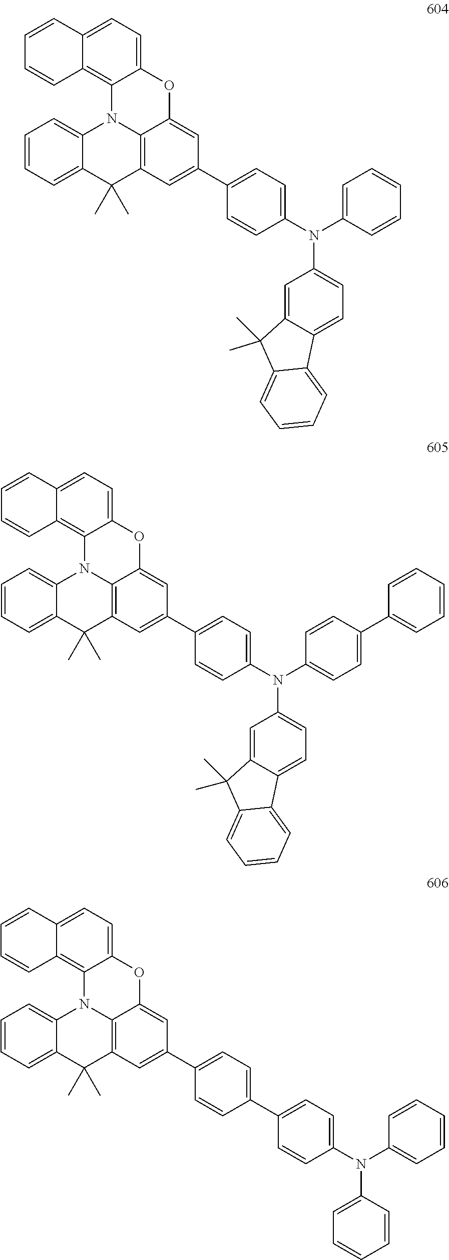 [SMILES: CC1(C)c2ccccc2-c2ccc(N(c3ccccc3)c3ccc(-c4cc5c6c(c4)C(C)(C)c4ccccc4N6c4c(ccc6ccccc46)O5)cc3)cc21 ; CC1(C)c2ccccc2-c2ccc(N(c3ccc(-c4ccccc4)cc3)c3ccc(-c4cc5c6c(c4)C(C)(C)c4ccccc4N6c4c(ccc6ccccc46)O5)cc3)cc21 ; CC1(C)c2ccccc2N2c3c(cc(-c4ccc(-c5ccc(N(c6ccccc6)c6ccccc6)cc5)cc4)cc31)Oc1ccc3ccccc3c12]

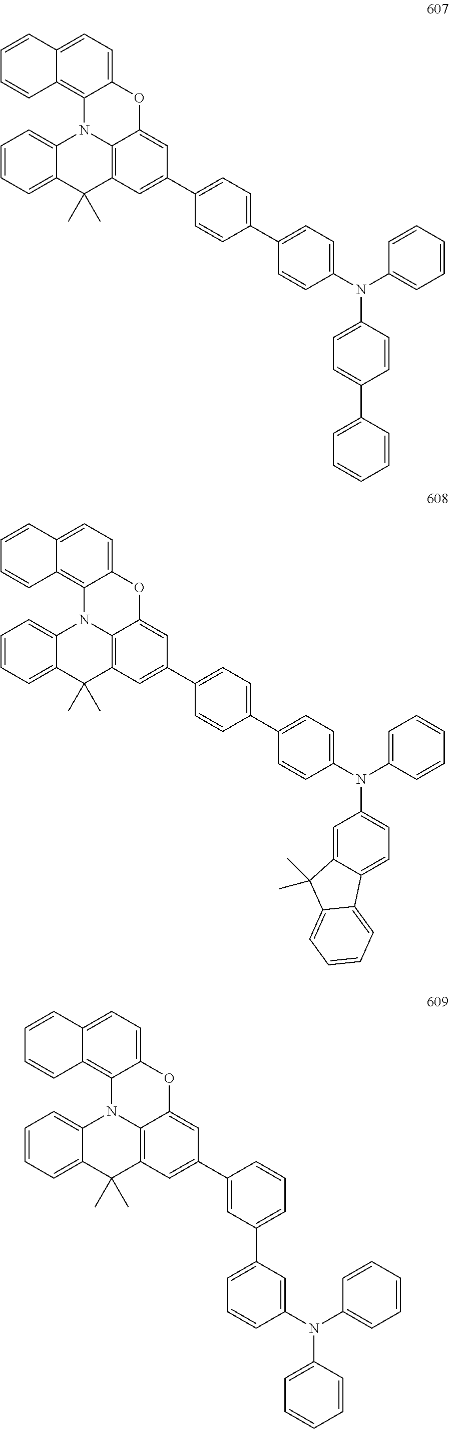 [SMILES: CC1(C)c2ccccc2N2c3c(cc(-c4ccc(-c5ccc(N(c6ccccc6)c6ccc(-c7ccccc7)cc6)cc5)cc4)cc31)Oc1ccc3ccccc3c12 ; CC1(C)c2ccccc2-c2ccc(N(c3ccccc3)c3ccc(-c4ccc(-c5cc6c7c(c5)C(C)(C)c5ccccc5N7c5c(ccc7ccccc57)O6)cc4)cc3)cc21 ; CC1(C)c2ccccc2N2c3c(cc(-c4cccc(-c5cccc(N(c6ccccc6)c6ccccc6)c5)c4)cc31)Oc1ccc3ccccc3c12]

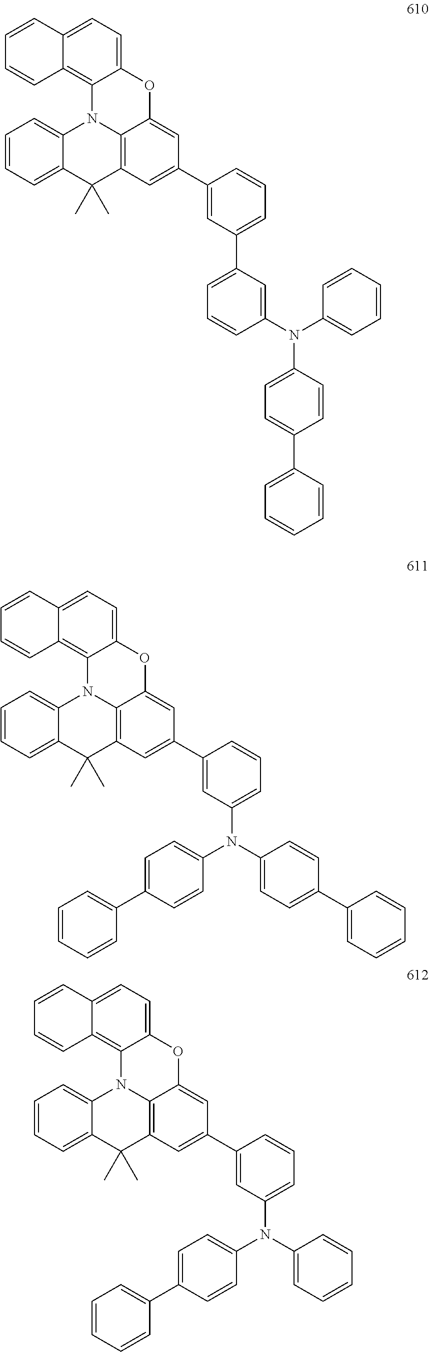

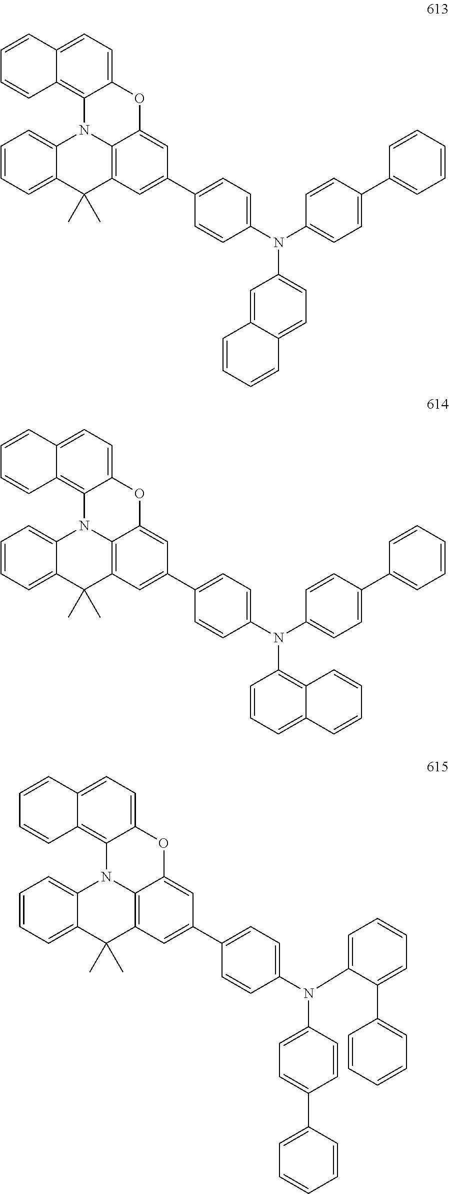 [SMILES: CC1(C)c2ccccc2N2c3c(cc(-c4ccc(N(c5ccc(-c6ccccc6)cc5)c5ccc6ccccc6c5)cc4)cc31)Oc1ccc3ccccc3c12 ; CC1(C)c2ccccc2N2c3c(cc(-c4ccc(N(c5ccc(-c6ccccc6)cc5)c5cccc6ccccc56)cc4)cc31)Oc1ccc3ccccc3c12 ; CC1(C)c2ccccc2N2c3c(cc(-c4ccc(N(c5ccc(-c6ccccc6)cc5)c5ccccc5-c5ccccc5)cc4)cc31)Oc1ccc3ccccc3c12]

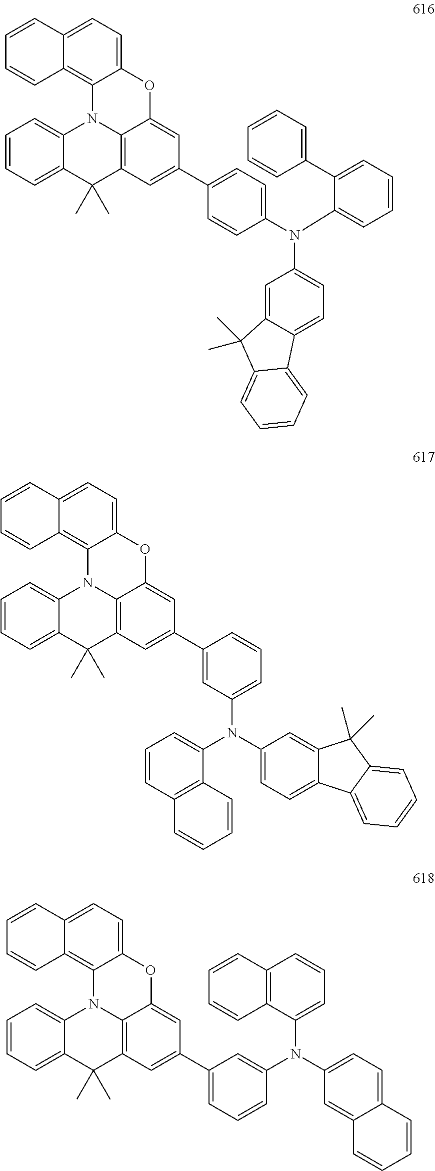 [SMILES: CC1(C)c2ccccc2-c2ccc(N(c3ccc(-c4cc5c6c(c4)C(C)(C)c4ccccc4N6c4c(ccc6ccccc46)O5)cc3)c3ccccc3-c3ccccc3)cc21 ; CC1(C)c2ccccc2-c2ccc(N(c3cccc(-c4cc5c6c(c4)C(C)(C)c4ccccc4N6c4c(ccc6ccccc46)O5)c3)c3cccc4ccccc34)cc21 ; CC1(C)c2ccccc2N2c3c(cc(-c4cccc(N(c5ccc6ccccc6c5)c5cccc6ccccc56)c4)cc31)Oc1ccc3ccccc3c12]

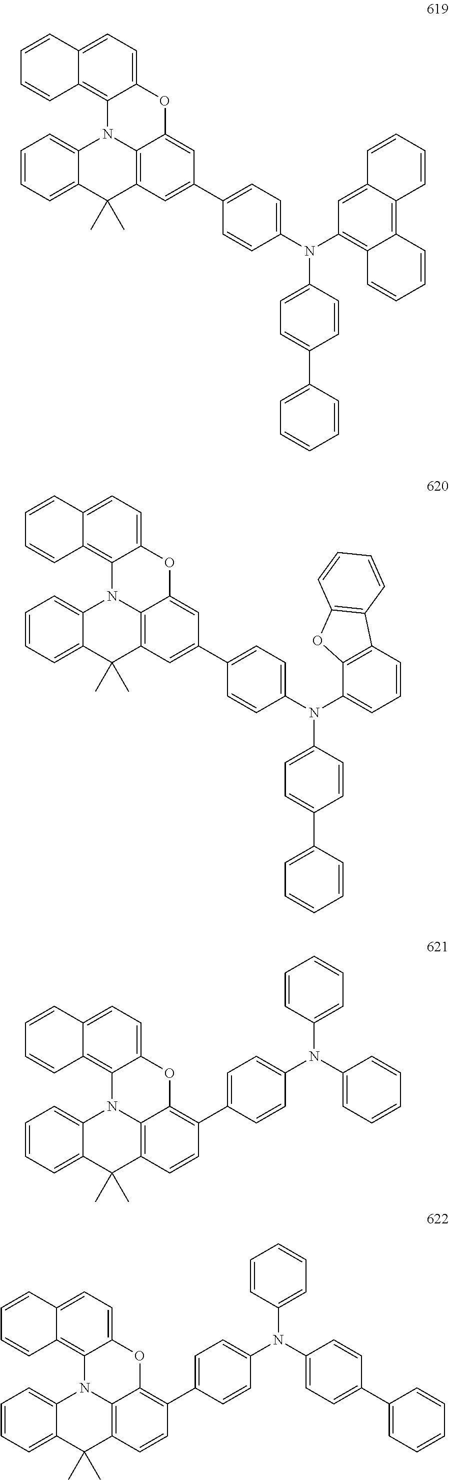

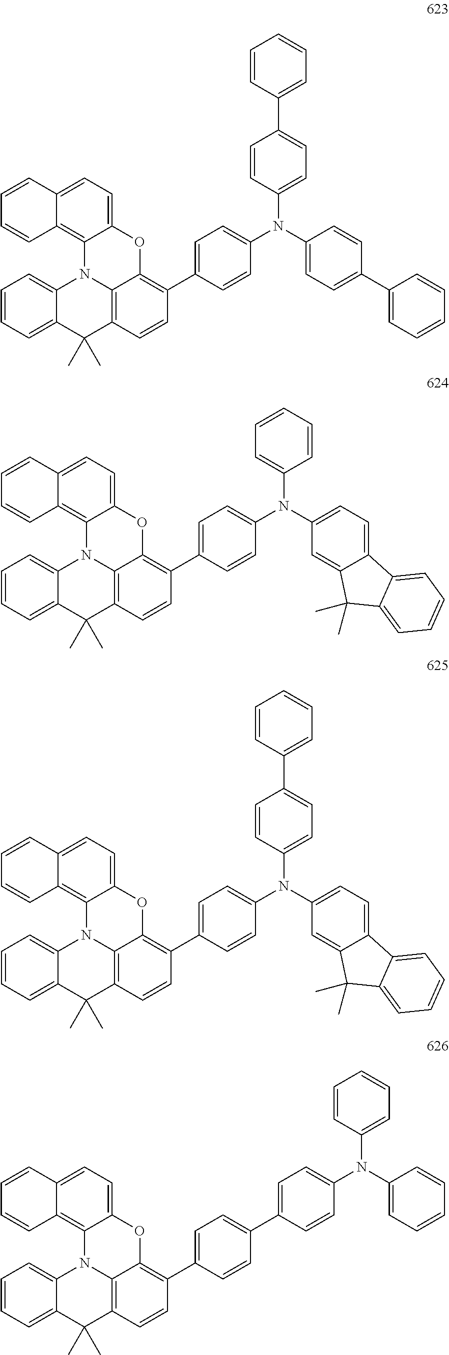 [SMILES: CC1(C)c2ccccc2N2c3c1ccc(-c1ccc(N(c4ccc(-c5ccccc5)cc4)c4ccc(-c5ccccc5)cc4)cc1)c3Oc1ccc3ccccc3c12 ; CC1(C)c2ccccc2-c2ccc(N(c3ccccc3)c3ccc(-c4ccc5c6c4Oc4ccc7ccccc7c4N6c4ccccc4C5(C)C)cc3)cc21 ; CC1(C)c2ccccc2-c2ccc(N(c3ccc(-c4ccccc4)cc3)c3ccc(-c4ccc5c6c4Oc4ccc7ccccc7c4N6c4ccccc4C5(C)C)cc3)cc21 ; CC1(C)c2ccccc2N2c3c1ccc(-c1ccc(-c4ccc(N(c5ccccc5)c5ccccc5)cc4)cc1)c3Oc1ccc3ccccc3c12]

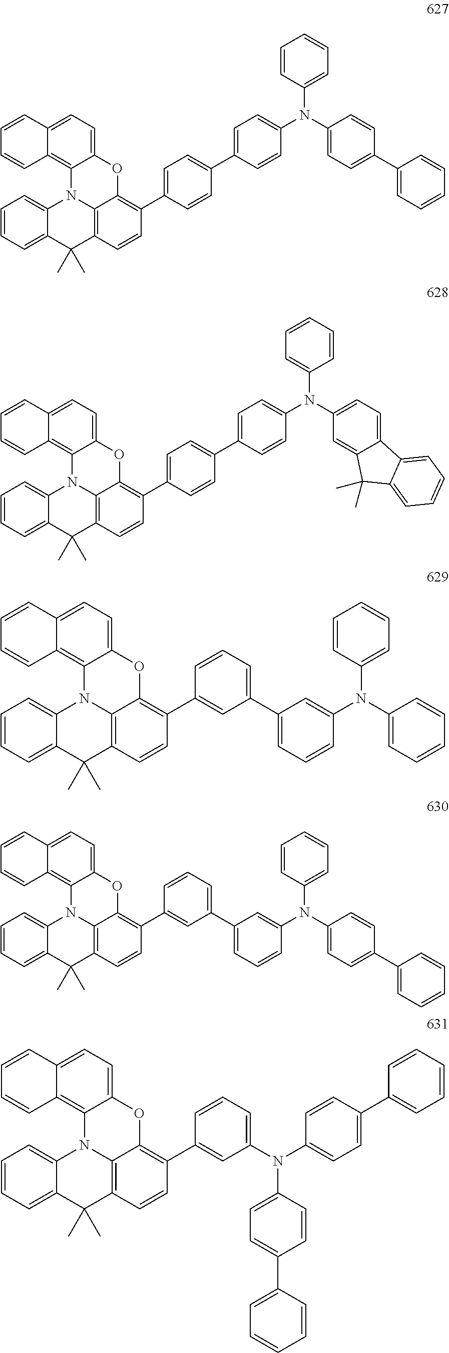 [SMILES: CC1(C)c2ccccc2N2c3c1ccc(-c1ccc(-c4ccc(N(c5ccccc5)c5ccc(-c6ccccc6)cc5)cc4)cc1)c3Oc1ccc3ccccc3c12 ; CC1(C)c2ccccc2-c2ccc(N(c3ccccc3)c3ccc(-c4ccc(-c5ccc6c7c5Oc5ccc8ccccc8c5N7c5ccccc5C6(C)C)cc4)cc3)cc21 ; CC1(C)c2ccccc2N2c3c1ccc(-c1cccc(-c4cccc(N(c5ccccc5)c5ccccc5)c4)c1)c3Oc1ccc3ccccc3c12 ; CC1(C)c2ccccc2N2c3c1ccc(-c1cccc(-c4cccc(N(c5ccccc5)c5ccc(-c6ccccc6)cc5)c4)c1)c3Oc1ccc3ccccc3c12 ; CC1(C)c2ccccc2N2c3c1ccc(-c1cccc(N(c4ccc(-c5ccccc5)cc4)c4ccc(-c5ccccc5)cc4)c1)c3Oc1ccc3ccccc3c12]

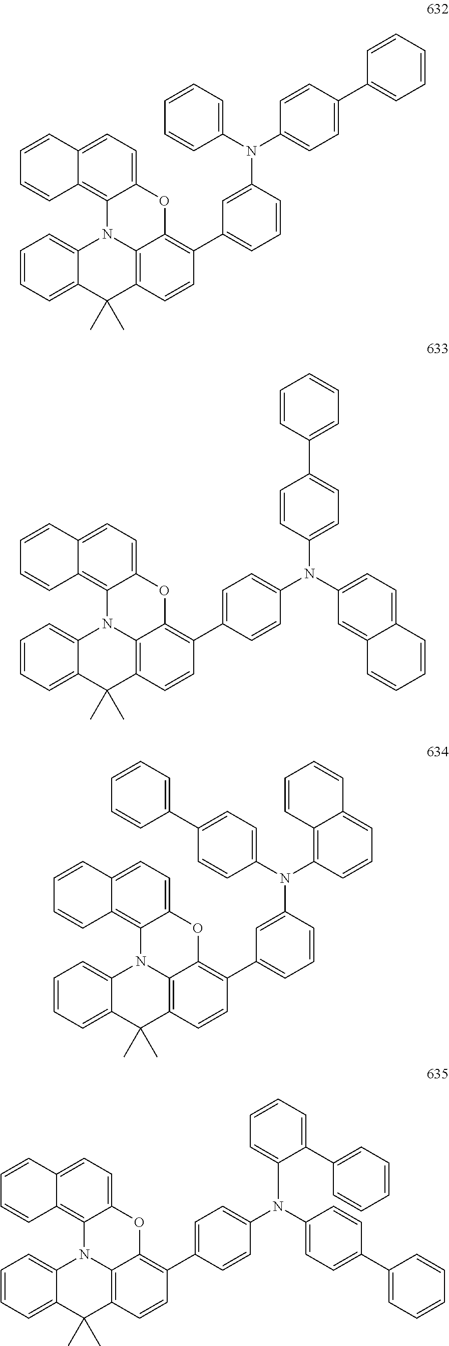

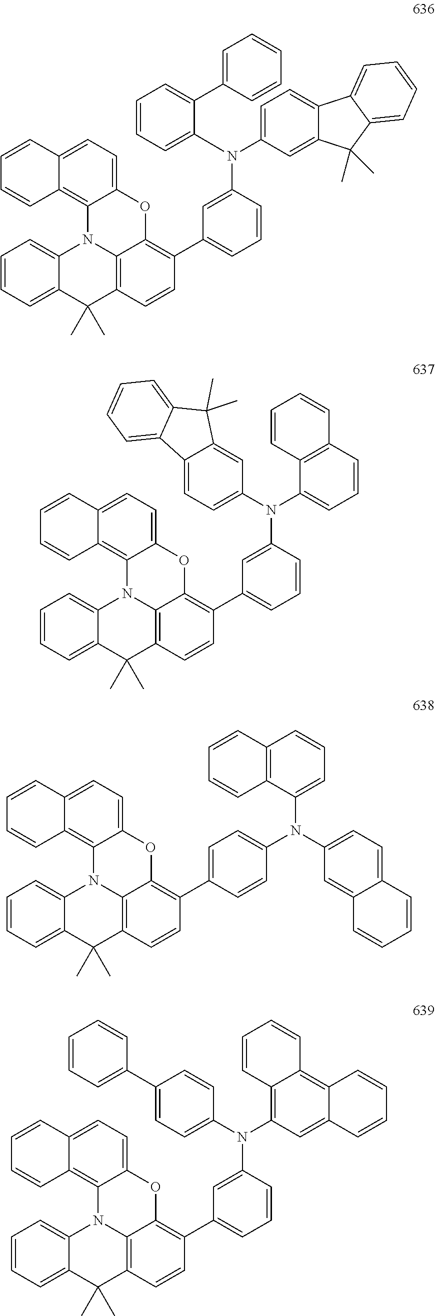

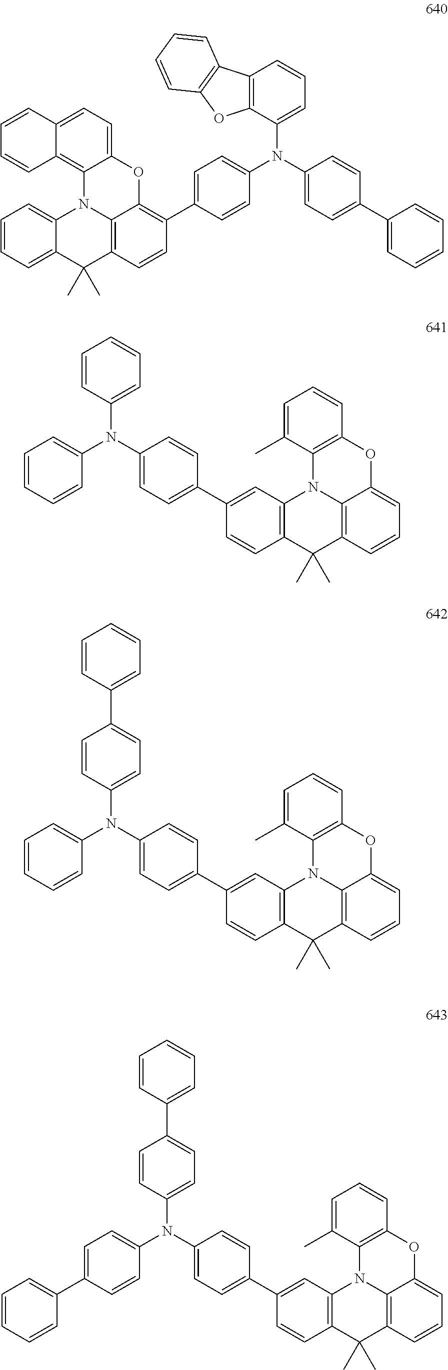

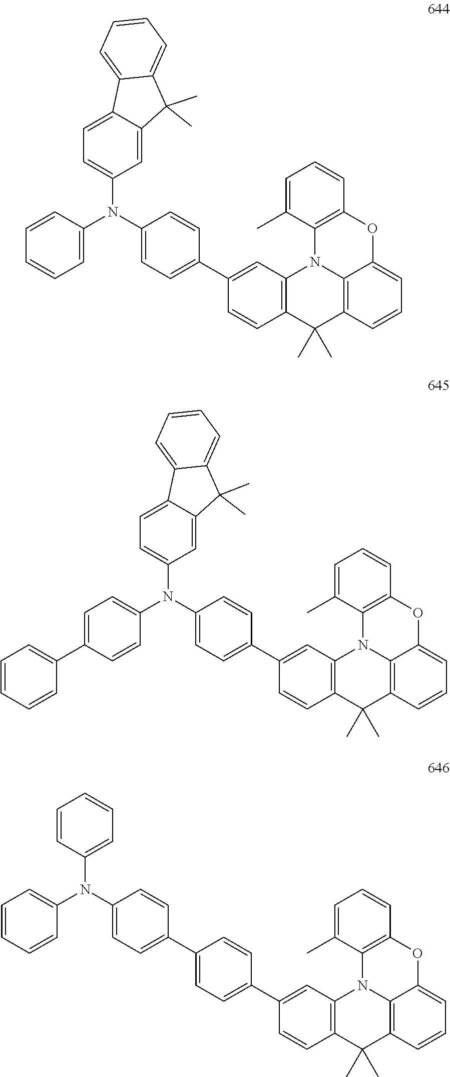 [SMILES: Cc1cccc2c1N1c3cc(-c4ccc(N(c5ccccc5)c5ccc6c(c5)C(C)(C)c5ccccc5-6)cc4)ccc3C(C)(C)c3cccc(c31)O2 ; Cc1cccc2c1N1c3cc(-c4ccc(N(c5ccc(-c6ccccc6)cc5)c5ccc6c(c5)C(C)(C)c5ccccc5-6)cc4)ccc3C(C)(C)c3cccc(c31)O2 ; Cc1cccc2c1N1c3cc(-c4ccc(-c5ccc(N(c6ccccc6)c6ccccc6)cc5)cc4)ccc3C(C)(C)c3cccc(c31)O2]

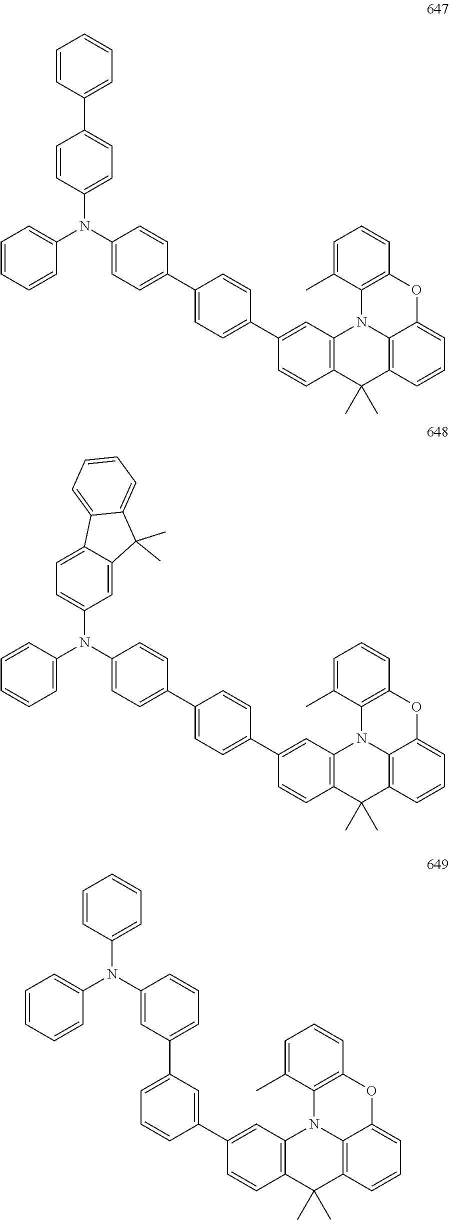 [SMILES: Cc1cccc2c1N1c3cc(-c4ccc(-c5ccc(N(c6ccccc6)c6ccc(-c7ccccc7)cc6)cc5)cc4)ccc3C(C)(C)c3cccc(c31)O2 ; Cc1cccc2c1N1c3cc(-c4ccc(-c5ccc(N(c6ccccc6)c6ccc7c(c6)C(C)(C)c6ccccc6-7)cc5)cc4)ccc3C(C)(C)c3cccc(c31)O2 ; Cc1cccc2c1N1c3cc(-c4cccc(-c5cccc(N(c6ccccc6)c6ccccc6)c5)c4)ccc3C(C)(C)c3cccc(c31)O2]

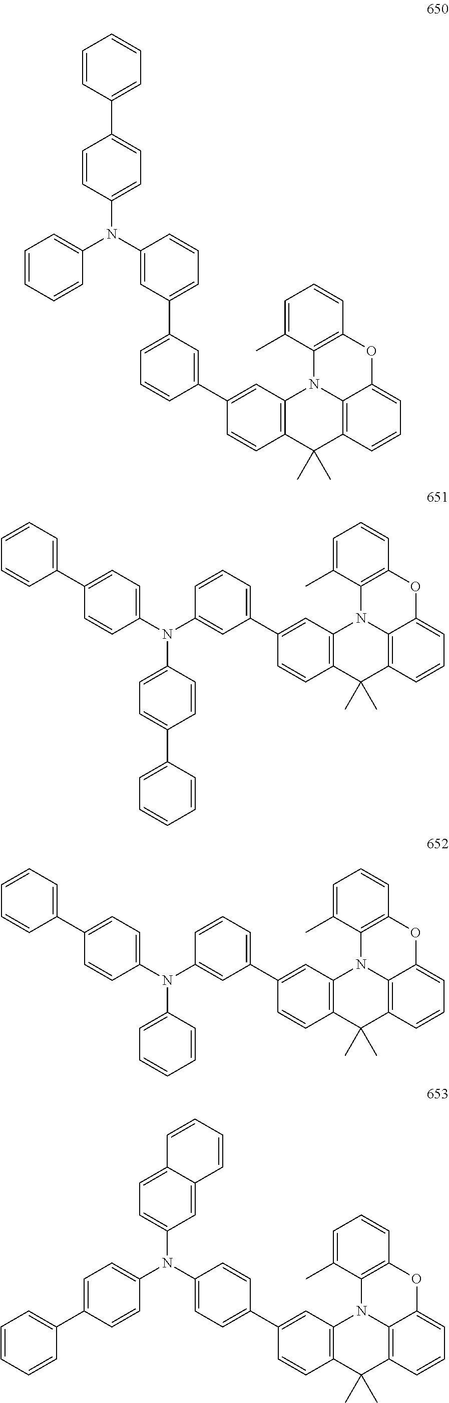 [SMILES: Cc1cccc2c1N1c3cc(-c4cccc(-c5cccc(N(c6ccccc6)c6ccc(-c7ccccc7)cc6)c5)c4)ccc3C(C)(C)c3cccc(c31)O2 ; Cc1cccc2c1N1c3cc(-c4cccc(N(c5ccc(-c6ccccc6)cc5)c5ccc(-c6ccccc6)cc5)c4)ccc3C(C)(C)c3cccc(c31)O2 ; Cc1cccc2c1N1c3cc(-c4cccc(N(c5ccccc5)c5ccc(-c6ccccc6)cc5)c4)ccc3C(C)(C)c3cccc(c31)O2 ; Cc1cccc2c1N1c3cc(-c4ccc(N(c5ccc(-c6ccccc6)cc5)c5ccc6ccccc6c5)cc4)ccc3C(C)(C)c3cccc(c31)O2]

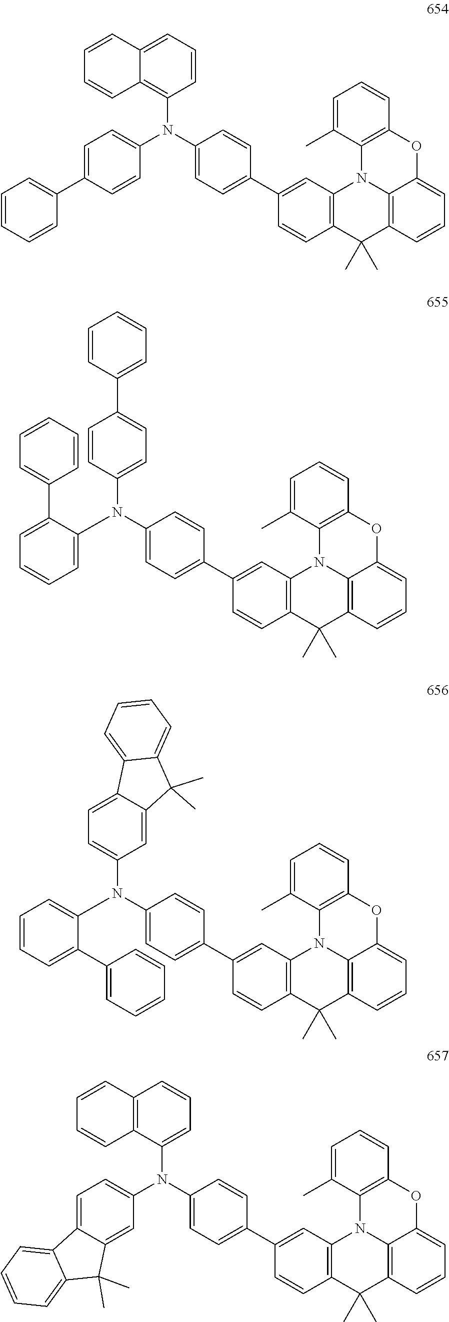 [SMILES: Cc1cccc2c1N1c3cc(-c4ccc(N(c5ccc(-c6ccccc6)cc5)c5cccc6ccccc56)cc4)ccc3C(C)(C)c3cccc(c31)O2 ; Cc1cccc2c1N1c3cc(-c4ccc(N(c5ccc(-c6ccccc6)cc5)c5ccccc5-c5ccccc5)cc4)ccc3C(C)(C)c3cccc(c31)O2 ; Cc1cccc2c1N1c3cc(-c4ccc(N(c5ccc6c(c5)C(C)(C)c5ccccc5-6)c5ccccc5-c5ccccc5)cc4)ccc3C(C)(C)c3cccc(c31)O2 ; Cc1cccc2c1N1c3cc(-c4ccc(N(c5ccc6c(c5)C(C)(C)c5ccccc5-6)c5cccc6ccccc56)cc4)ccc3C(C)(C)c3cccc(c31)O2]

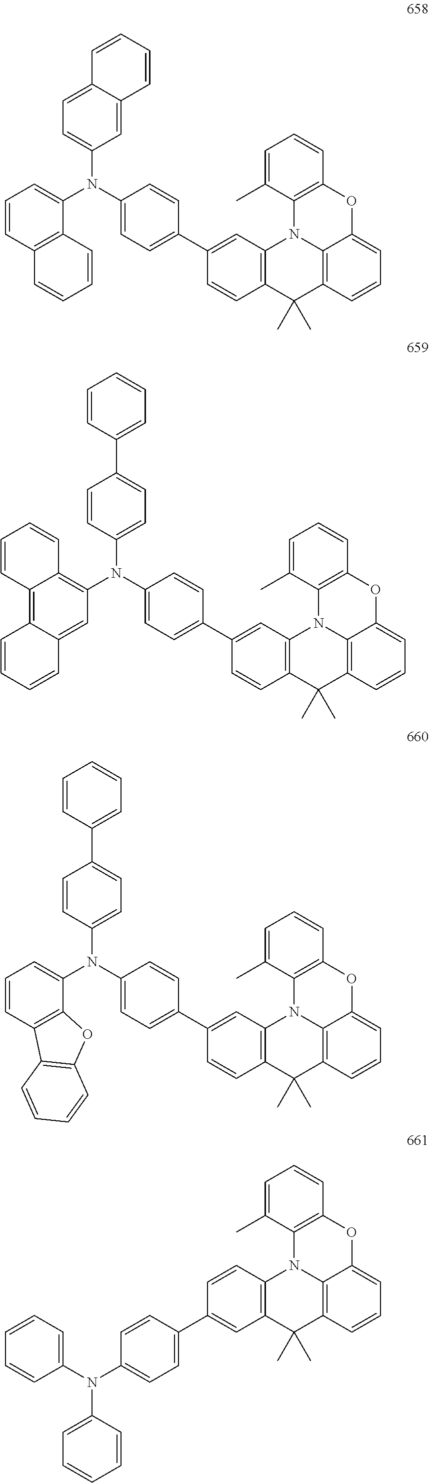 [SMILES: Cc1cccc2c1N1c3cc(-c4ccc(N(c5ccc6ccccc6c5)c5cccc6ccccc56)cc4)ccc3C(C)(C)c3cccc(c31)O2 ; Cc1cccc2c1N1c3cc(-c4ccc(N(c5ccc(-c6ccccc6)cc5)c5cc6ccccc6c6ccccc56)cc4)ccc3C(C)(C)c3cccc(c31)O2 ; Cc1cccc2c1N1c3cc(-c4ccc(N(c5ccc(-c6ccccc6)cc5)c5cccc6c5oc5ccccc56)cc4)ccc3C(C)(C)c3cccc(c31)O2 ; Cc1cccc2c1N1c3ccc(-c4ccc(N(c5ccccc5)c5ccccc5)cc4)cc3C(C)(C)c3cccc(c31)O2]

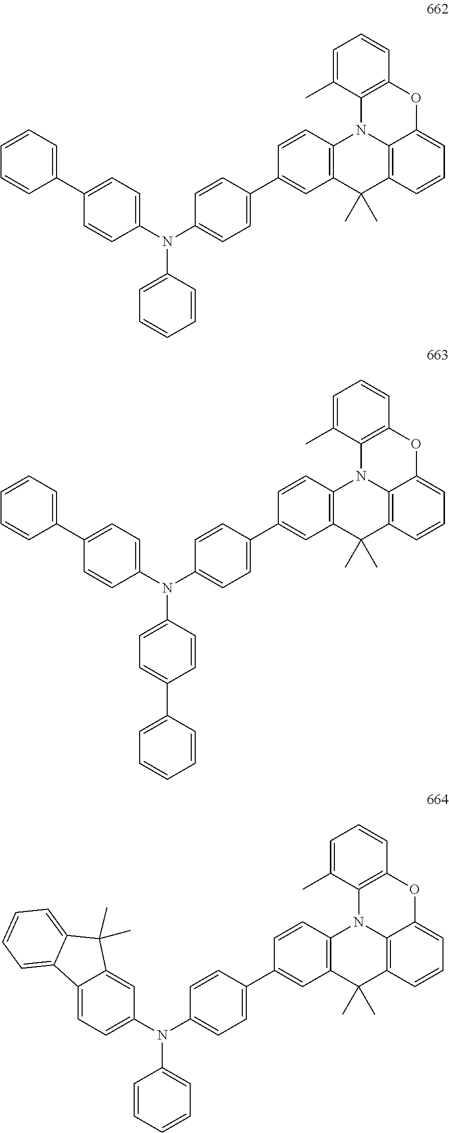 [SMILES: Cc1cccc2c1N1c3ccc(-c4ccc(N(c5ccccc5)c5ccc(-c6ccccc6)cc5)cc4)cc3C(C)(C)c3cccc(c31)O2 ; Cc1cccc2c1N1c3ccc(-c4ccc(N(c5ccc(-c6ccccc6)cc5)c5ccc(-c6ccccc6)cc5)cc4)cc3C(C)(C)c3cccc(c31)O2 ; Cc1cccc2c1N1c3ccc(-c4ccc(N(c5ccccc5)c5ccc6c(c5)C(C)(C)c5ccccc5-6)cc4)cc3C(C)(C)c3cccc(c31)O2]

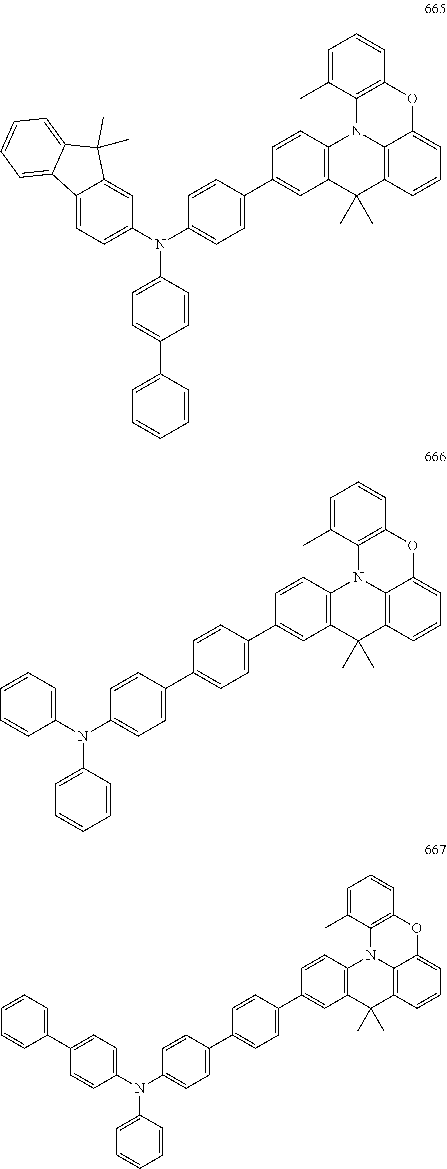 [SMILES: Cc1cccc2c1N1c3ccc(-c4ccc(N(c5ccc(-c6ccccc6)cc5)c5ccc6c(c5)C(C)(C)c5ccccc5-6)cc4)cc3C(C)(C)c3cccc(c31)O2 ; Cc1cccc2c1N1c3ccc(-c4ccc(-c5ccc(N(c6ccccc6)c6ccccc6)cc5)cc4)cc3C(C)(C)c3cccc(c31)O2 ; Cc1cccc2c1N1c3ccc(-c4ccc(-c5ccc(N(c6ccccc6)c6ccc(-c7ccccc7)cc6)cc5)cc4)cc3C(C)(C)c3cccc(c31)O2]

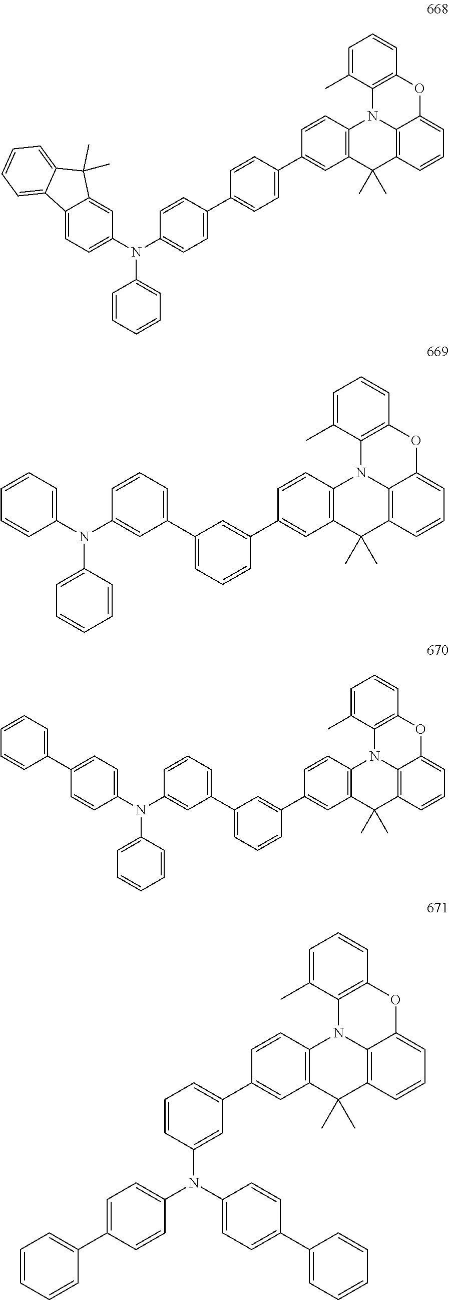 [SMILES: Cc1cccc2c1N1c3ccc(-c4ccc(-c5ccc(N(c6ccccc6)c6ccc7c(c6)C(C)(C)c6ccccc6-7)cc5)cc4)cc3C(C)(C)c3cccc(c31)O2 ; Cc1cccc2c1N1c3ccc(-c4cccc(-c5cccc(N(c6ccccc6)c6ccccc6)c5)c4)cc3C(C)(C)c3cccc(c31)O2 ; Cc1cccc2c1N1c3ccc(-c4cccc(-c5cccc(N(c6ccccc6)c6ccc(-c7ccccc7)cc6)c5)c4)cc3C(C)(C)c3cccc(c31)O2 ; Cc1cccc2c1N1c3ccc(-c4cccc(N(c5ccc(-c6ccccc6)cc5)c5ccc(-c6ccccc6)cc5)c4)cc3C(C)(C)c3cccc(c31)O2]

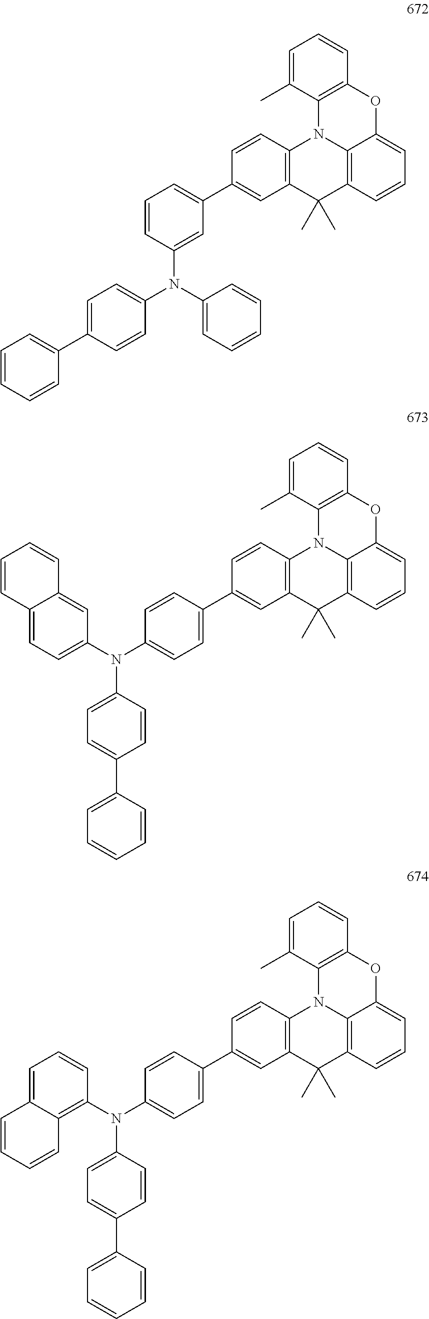 [SMILES: Cc1cccc2c1N1c3ccc(-c4cccc(N(c5ccccc5)c5ccc(-c6ccccc6)cc5)c4)cc3C(C)(C)c3cccc(c31)O2 ; Cc1cccc2c1N1c3ccc(-c4ccc(N(c5ccc(-c6ccccc6)cc5)c5ccc6ccccc6c5)cc4)cc3C(C)(C)c3cccc(c31)O2 ; Cc1cccc2c1N1c3ccc(-c4ccc(N(c5ccc(-c6ccccc6)cc5)c5cccc6ccccc56)cc4)cc3C(C)(C)c3cccc(c31)O2]

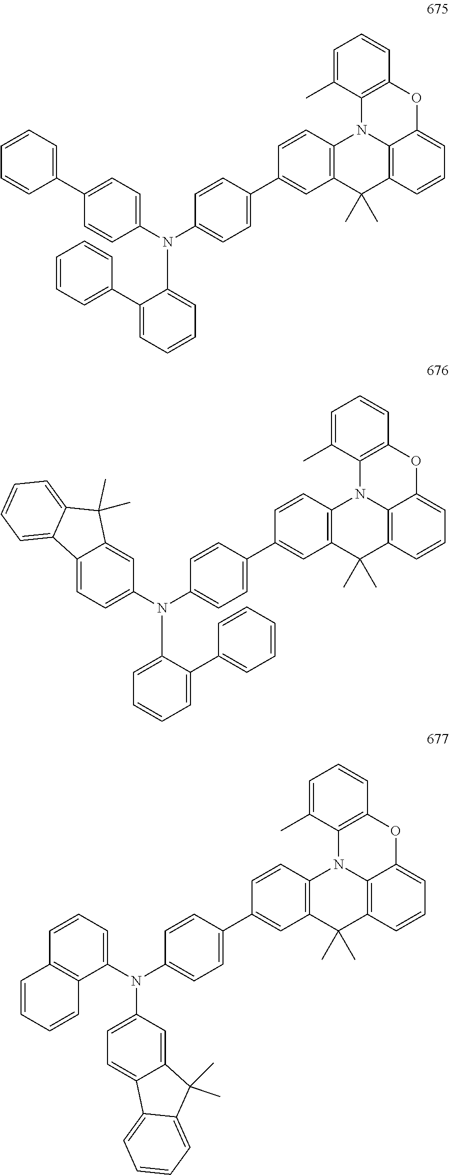 [SMILES: Cc1cccc2c1N1c3ccc(-c4ccc(N(c5ccc(-c6ccccc6)cc5)c5ccccc5-c5ccccc5)cc4)cc3C(C)(C)c3cccc(c31)O2 ; Cc1cccc2c1N1c3ccc(-c4ccc(N(c5ccc6c(c5)C(C)(C)c5ccccc5-6)c5ccccc5-c5ccccc5)cc4)cc3C(C)(C)c3cccc(c31)O2 ; Cc1cccc2c1N1c3ccc(-c4ccc(N(c5ccc6c(c5)C(C)(C)c5ccccc5-6)c5cccc6ccccc56)cc4)cc3C(C)(C)c3cccc(c31)O2]

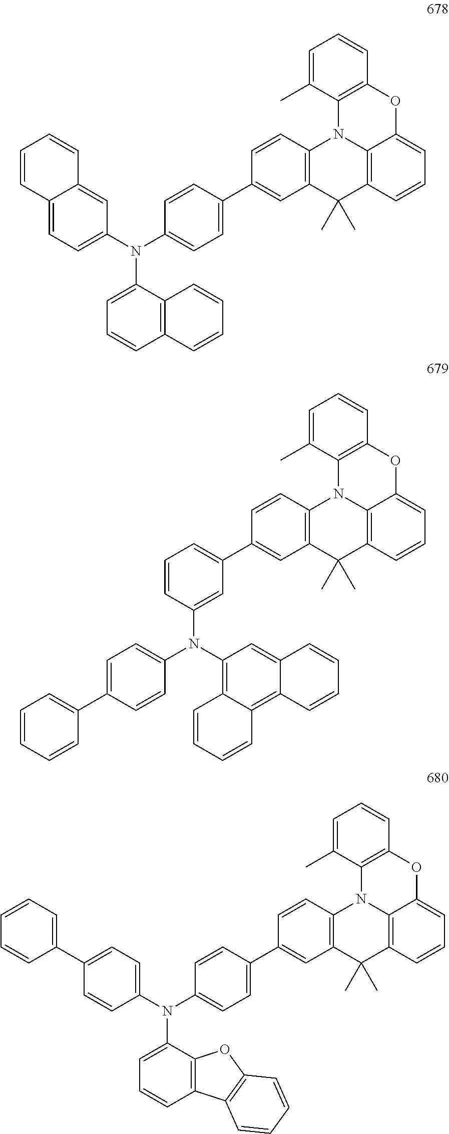 [SMILES: Cc1cccc2c1N1c3ccc(-c4ccc(N(c5ccc6ccccc6c5)c5cccc6ccccc56)cc4)cc3C(C)(C)c3cccc(c31)O2 ; Cc1cccc2c1N1c3ccc(-c4cccc(N(c5ccc(-c6ccccc6)cc5)c5cc6ccccc6c6ccccc56)c4)cc3C(C)(C)c3cccc(c31)O2 ; Cc1cccc2c1N1c3ccc(-c4ccc(N(c5ccc(-c6ccccc6)cc5)c5cccc6c5oc5ccccc56)cc4)cc3C(C)(C)c3cccc(c31)O2]

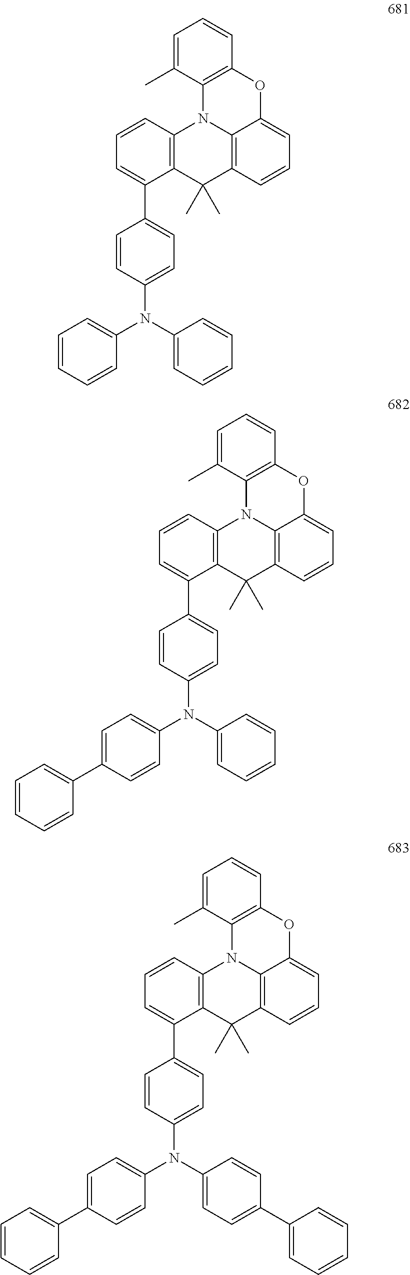 [SMILES: Cc1cccc2c1N1c3cccc(-c4ccc(N(c5ccccc5)c5ccccc5)cc4)c3C(C)(C)c3cccc(c31)O2 ; Cc1cccc2c1N1c3cccc(-c4ccc(N(c5ccccc5)c5ccc(-c6ccccc6)cc5)cc4)c3C(C)(C)c3cccc(c31)O2 ; Cc1cccc2c1N1c3cccc(-c4ccc(N(c5ccc(-c6ccccc6)cc5)c5ccc(-c6ccccc6)cc5)cc4)c3C(C)(C)c3cccc(c31)O2]

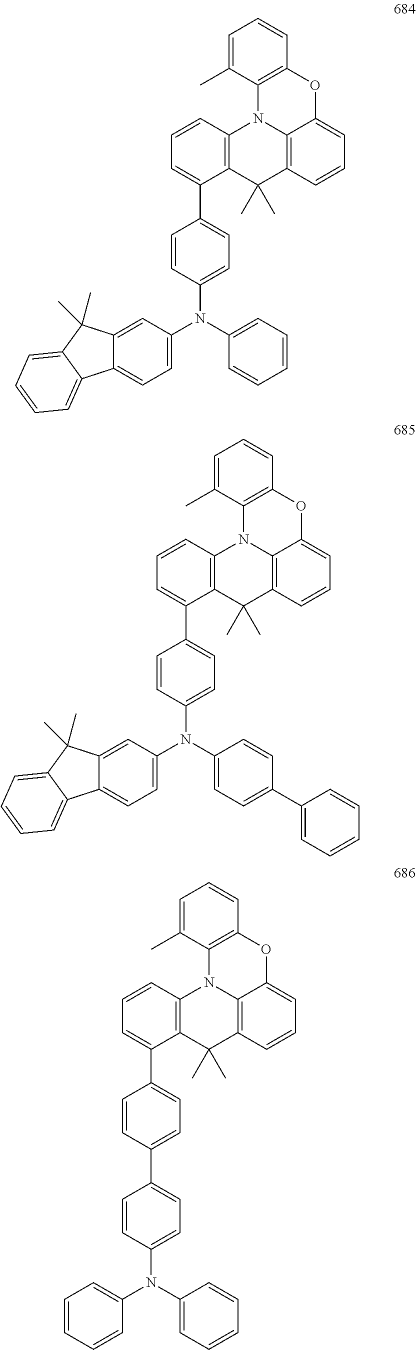 [SMILES: Cc1cccc2c1N1c3cccc(-c4ccc(N(c5ccccc5)c5ccc6c(c5)C(C)(C)c5ccccc5-6)cc4)c3C(C)(C)c3cccc(c31)O2 ; Cc1cccc2c1N1c3cccc(-c4ccc(N(c5ccc(-c6ccccc6)cc5)c5ccc6c(c5)C(C)(C)c5ccccc5-6)cc4)c3C(C)(C)c3cccc(c31)O2 ; Cc1cccc2c1N1c3cccc(-c4ccc(-c5ccc(N(c6ccccc6)c6ccccc6)cc5)cc4)c3C(C)(C)c3cccc(c31)O2]

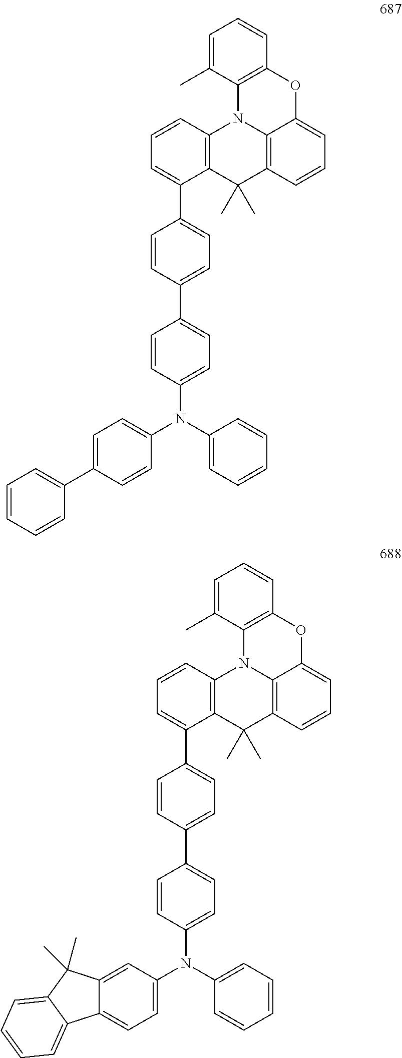 [SMILES: Cc1cccc2c1N1c3cccc(-c4ccc(-c5ccc(N(c6ccccc6)c6ccc(-c7ccccc7)cc6)cc5)cc4)c3C(C)(C)c3cccc(c31)O2 ; Cc1cccc2c1N1c3cccc(-c4ccc(-c5ccc(N(c6ccccc6)c6ccc7c(c6)C(C)(C)c6ccccc6-7)cc5)cc4)c3C(C)(C)c3cccc(c31)O2]

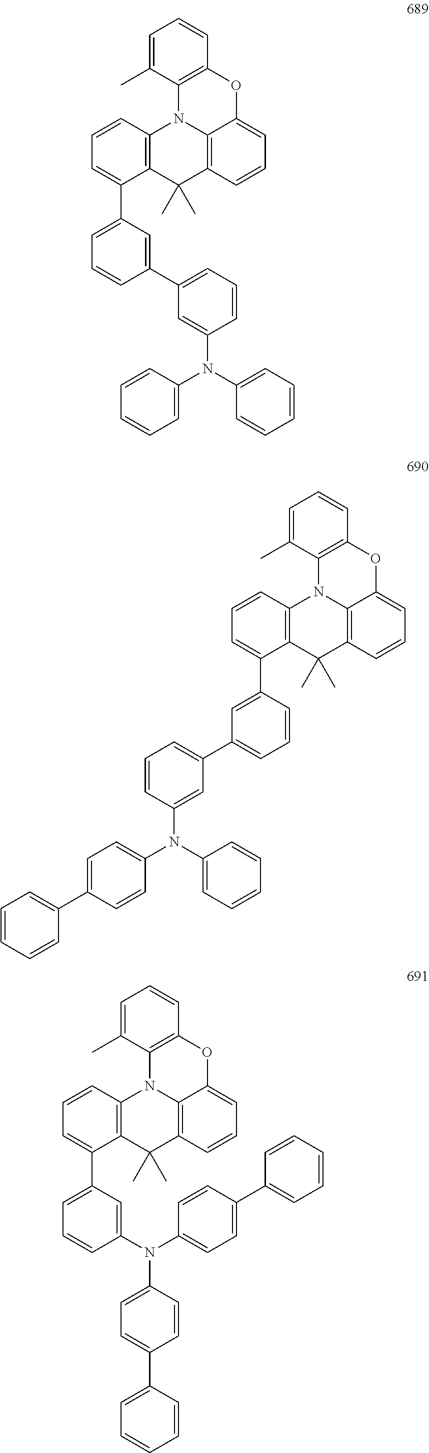 [SMILES: Cc1cccc2c1N1c3cccc(-c4cccc(-c5cccc(N(c6ccccc6)c6ccccc6)c5)c4)c3C(C)(C)c3cccc(c31)O2 ; Cc1cccc2c1N1c3cccc(-c4cccc(-c5cccc(N(c6ccccc6)c6ccc(-c7ccccc7)cc6)c5)c4)c3C(C)(C)c3cccc(c31)O2 ; Cc1cccc2c1N1c3cccc(-c4cccc(N(c5ccc(-c6ccccc6)cc5)c5ccc(-c6ccccc6)cc5)c4)c3C(C)(C)c3cccc(c31)O2]

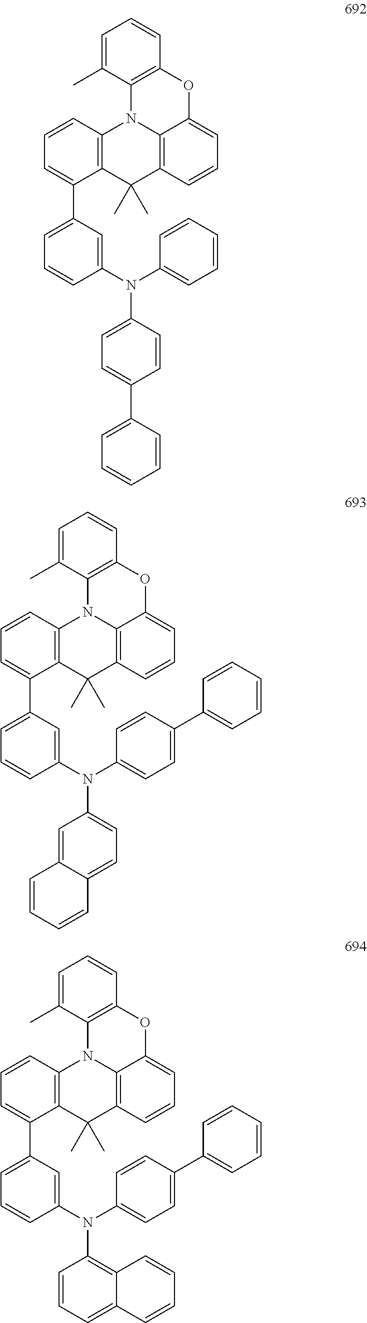

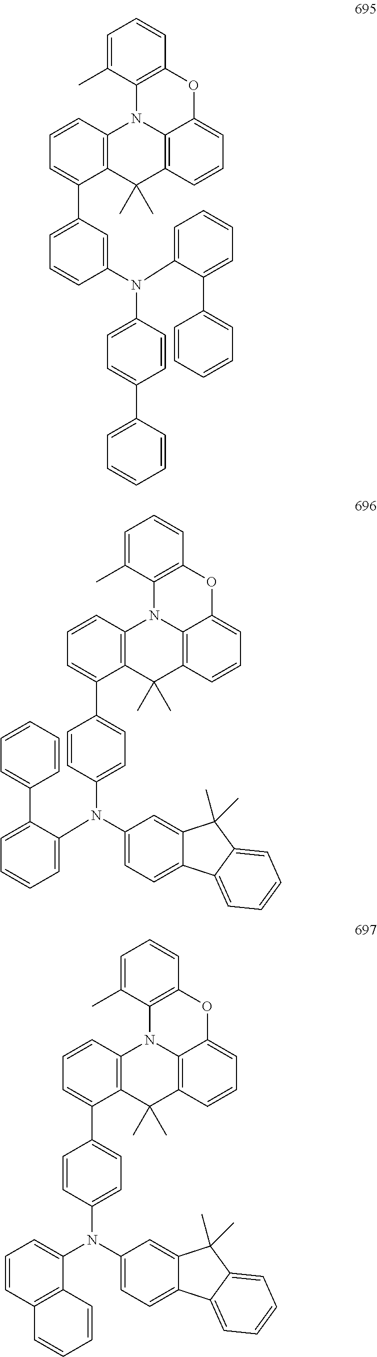

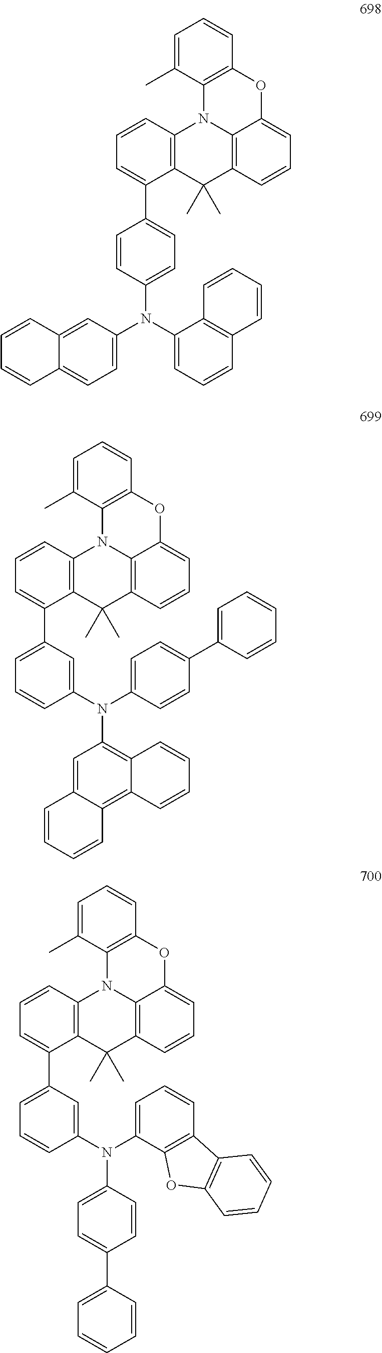

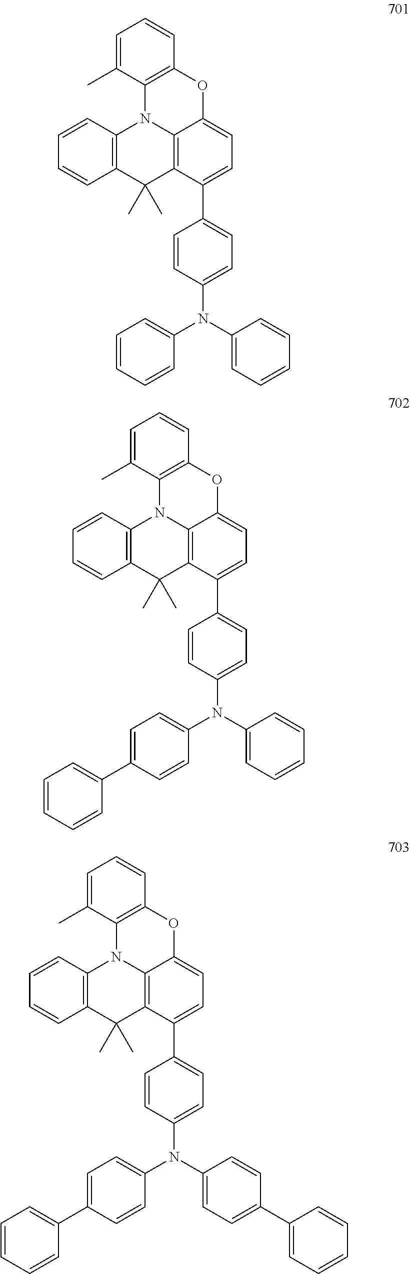

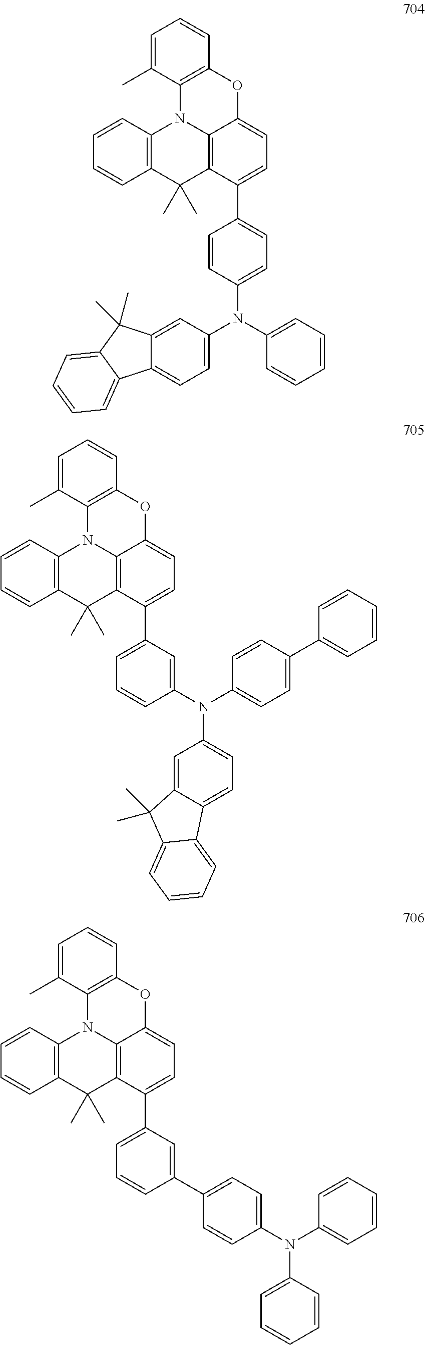

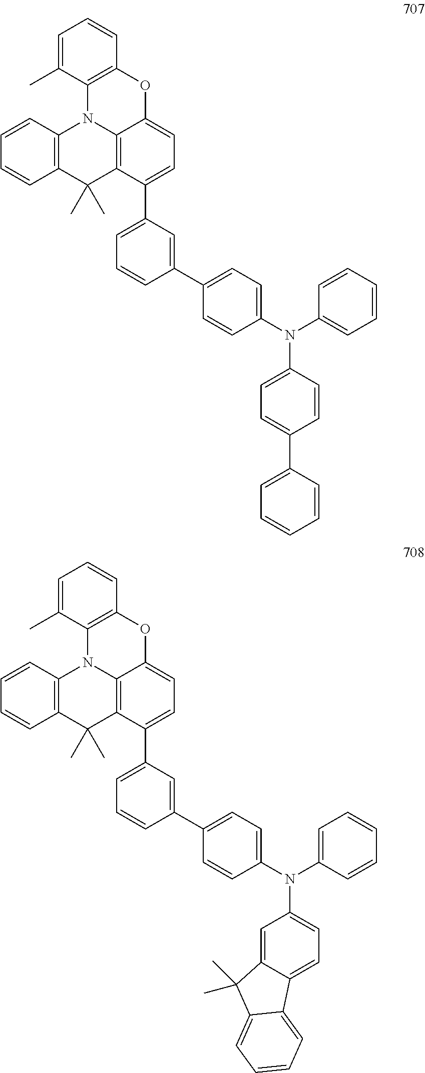 [SMILES: Cc1cccc2c1N1c3ccccc3C(C)(C)c3c(-c4cccc(-c5ccc(N(c6ccccc6)c6ccc(-c7ccccc7)cc6)cc5)c4)ccc(c31)O2 ; Cc1cccc2c1N1c3ccccc3C(C)(C)c3c(-c4cccc(-c5ccc(N(c6ccccc6)c6ccc7c(c6)C(C)(C)c6ccccc6-7)cc5)c4)ccc(c31)O2]

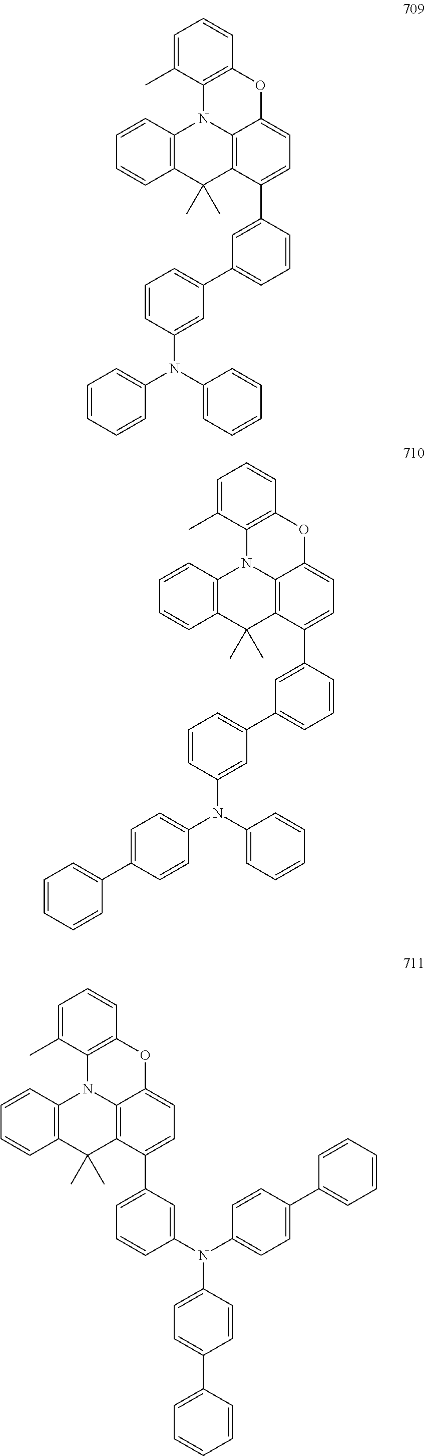

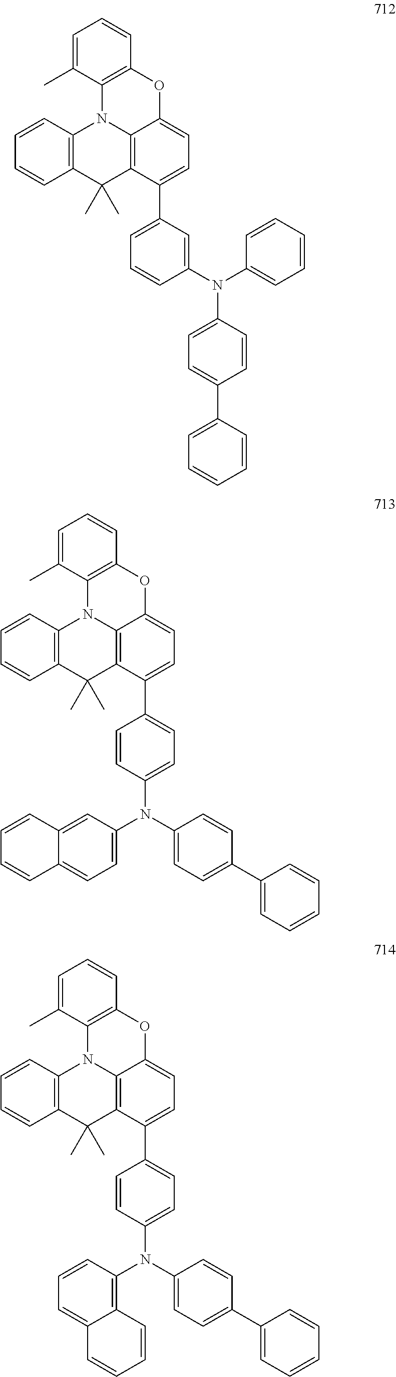 [SMILES: Cc1cccc2c1N1c3ccccc3C(C)(C)c3c(-c4cccc(N(c5ccccc5)c5ccc(-c6ccccc6)cc5)c4)ccc(c31)O2 ; Cc1cccc2c1N1c3ccccc3C(C)(C)c3c(-c4ccc(N(c5ccc(-c6ccccc6)cc5)c5ccc6ccccc6c5)cc4)ccc(c31)O2 ; Cc1cccc2c1N1c3ccccc3C(C)(C)c3c(-c4ccc(N(c5ccc(-c6ccccc6)cc5)c5cccc6ccccc56)cc4)ccc(c31)O2]

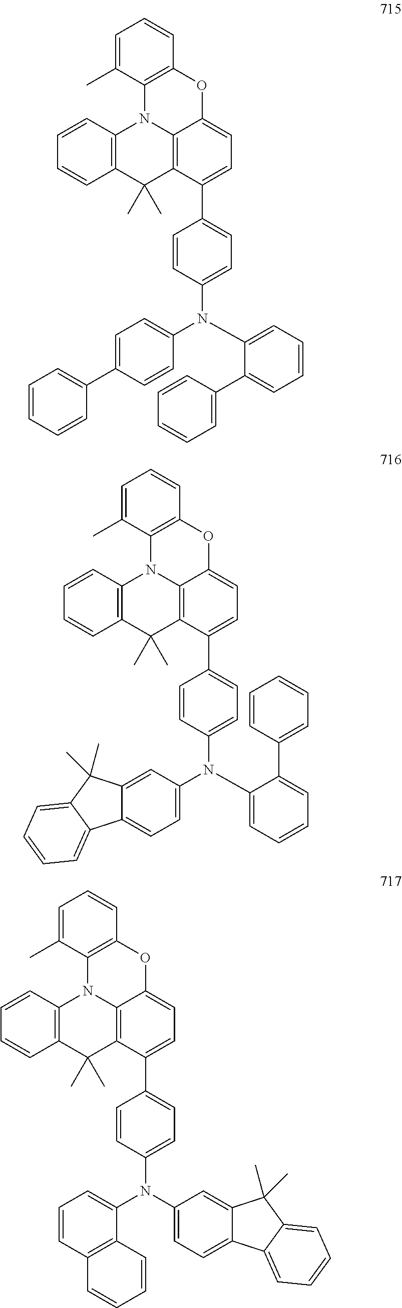

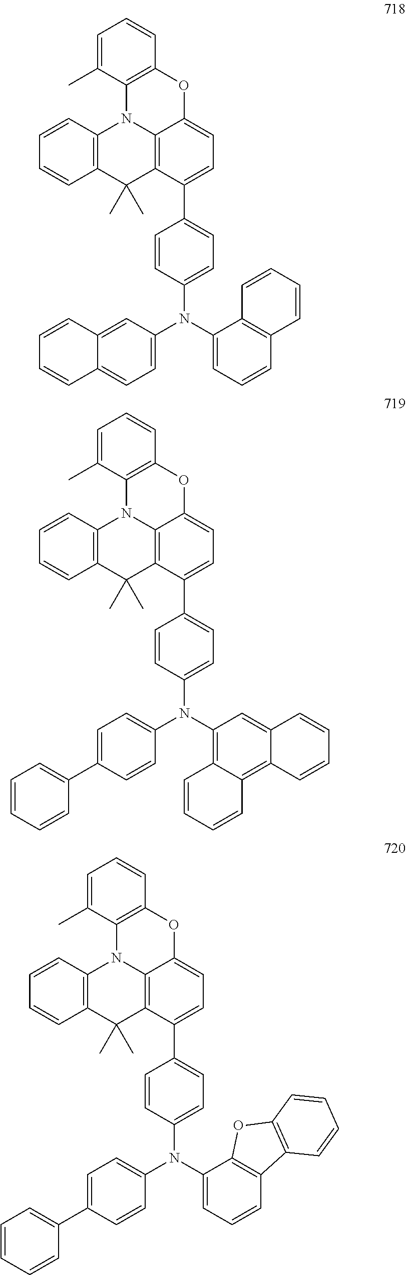

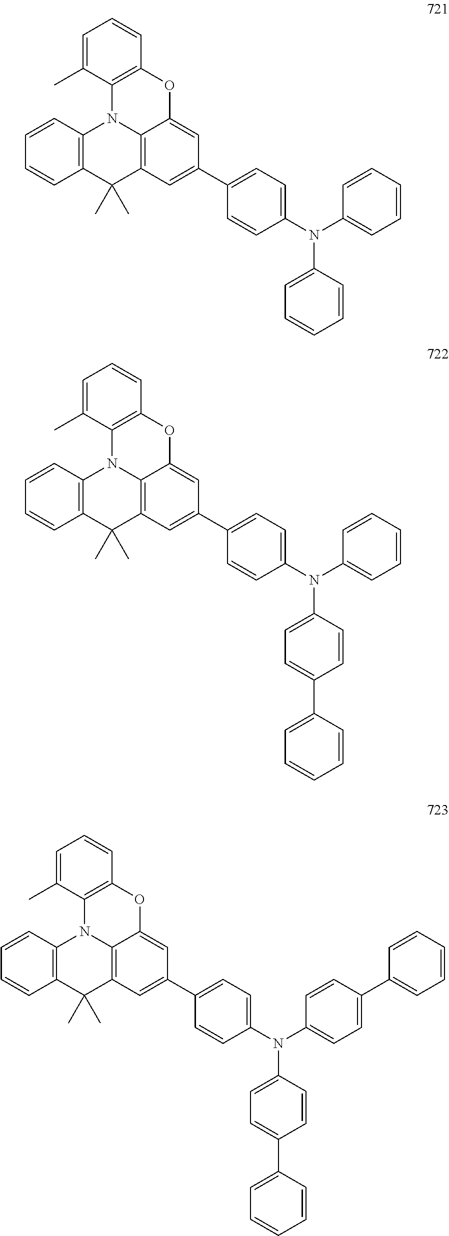 [SMILES: Cc1cccc2c1N1c3ccccc3C(C)(C)c3cc(-c4ccc(N(c5ccccc5)c5ccccc5)cc4)cc(c31)O2 ; Cc1cccc2c1N1c3ccccc3C(C)(C)c3cc(-c4ccc(N(c5ccccc5)c5ccc(-c6ccccc6)cc5)cc4)cc(c31)O2 ; Cc1cccc2c1N1c3ccccc3C(C)(C)c3cc(-c4ccc(N(c5ccc(-c6ccccc6)cc5)c5ccc(-c6ccccc6)cc5)cc4)cc(c31)O2]

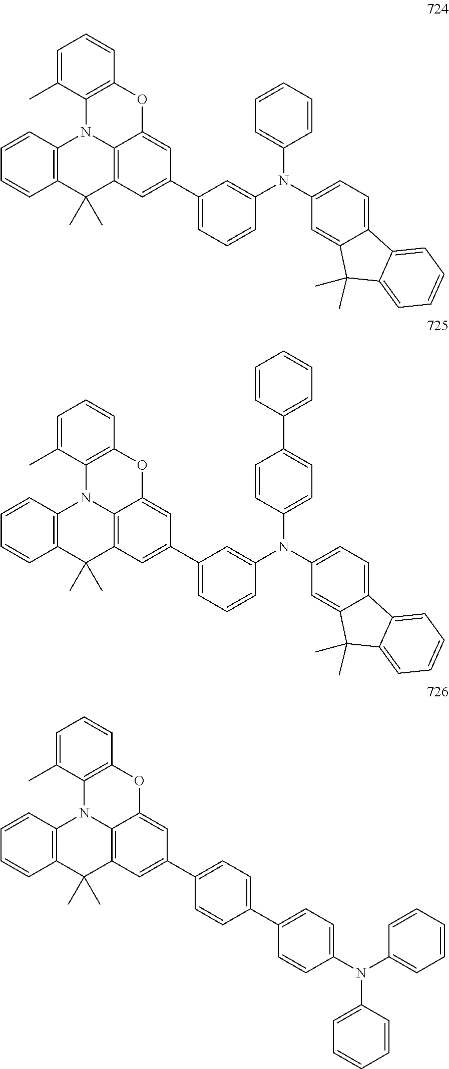 [SMILES: Cc1cccc2c1N1c3ccccc3C(C)(C)c3cc(-c4cccc(N(c5ccccc5)c5ccc6c(c5)C(C)(C)c5ccccc5-6)c4)cc(c31)O2 ; Cc1cccc2c1N1c3ccccc3C(C)(C)c3cc(-c4cccc(N(c5ccc(-c6ccccc6)cc5)c5ccc6c(c5)C(C)(C)c5ccccc5-6)c4)cc(c31)O2 ; Cc1cccc2c1N1c3ccccc3C(C)(C)c3cc(-c4ccc(-c5ccc(N(c6ccccc6)c6ccccc6)cc5)cc4)cc(c31)O2]

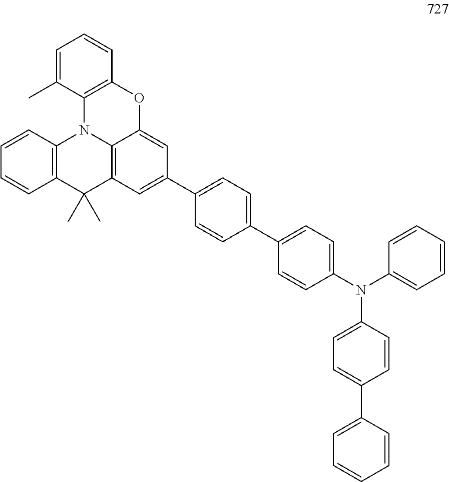 [SMILES: Cc1cccc2c1N1c3ccccc3C(C)(C)c3cc(-c4ccc(-c5ccc(N(c6ccccc6)c6ccc(-c7ccccc7)cc6)cc5)cc4)cc(c31)O2]

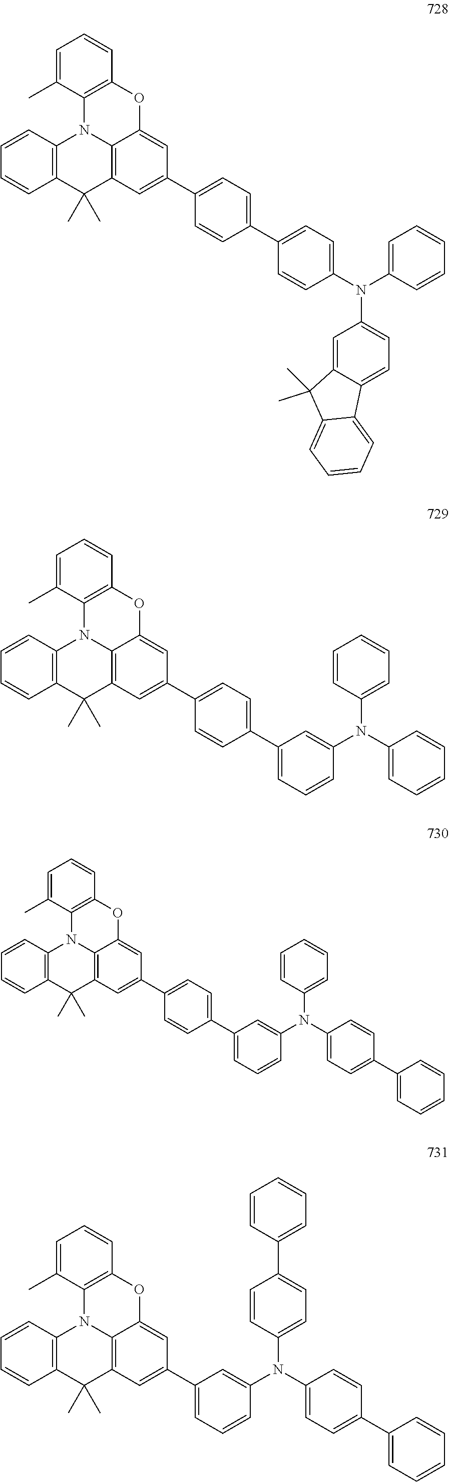 [SMILES: Cc1cccc2c1N1c3ccccc3C(C)(C)c3cc(-c4ccc(-c5ccc(N(c6ccccc6)c6ccc7c(c6)C(C)(C)c6ccccc6-7)cc5)cc4)cc(c31)O2 ; Cc1cccc2c1N1c3ccccc3C(C)(C)c3cc(-c4ccc(-c5cccc(N(c6ccccc6)c6ccccc6)c5)cc4)cc(c31)O2 ; Cc1cccc2c1N1c3ccccc3C(C)(C)c3cc(-c4ccc(-c5cccc(N(c6ccccc6)c6ccc(-c7ccccc7)cc6)c5)cc4)cc(c31)O2 ; Cc1cccc2c1N1c3ccccc3C(C)(C)c3cc(-c4cccc(N(c5ccc(-c6ccccc6)cc5)c5ccc(-c6ccccc6)cc5)c4)cc(c31)O2]

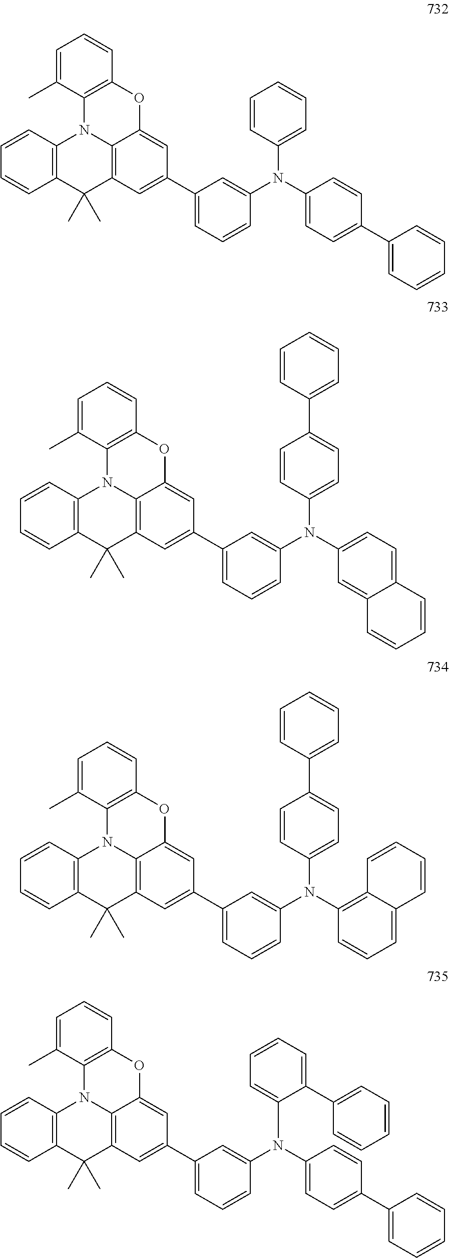 [SMILES: Cc1cccc2c1N1c3ccccc3C(C)(C)c3cc(-c4cccc(N(c5ccccc5)c5ccc(-c6ccccc6)cc5)c4)cc(c31)O2 ; Cc1cccc2c1N1c3ccccc3C(C)(C)c3cc(-c4cccc(N(c5ccc(-c6ccccc6)cc5)c5ccc6ccccc6c5)c4)cc(c31)O2 ; Cc1cccc2c1N1c3ccccc3C(C)(C)c3cc(-c4cccc(N(c5ccc(-c6ccccc6)cc5)c5cccc6ccccc56)c4)cc(c31)O2 ; Cc1cccc2c1N1c3ccccc3C(C)(C)c3cc(-c4cccc(N(c5ccc(-c6ccccc6)cc5)c5ccccc5-c5ccccc5)c4)cc(c31)O2]

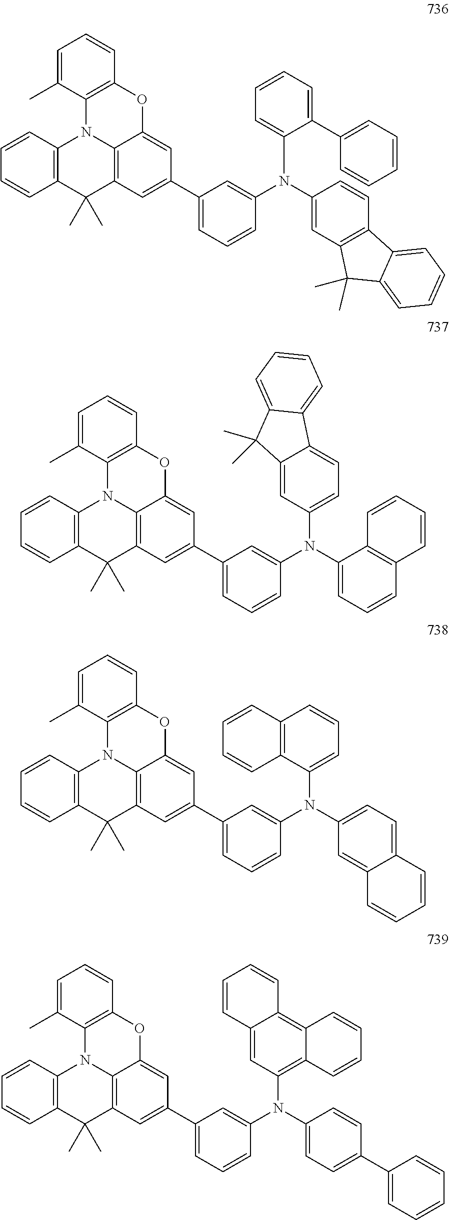 [SMILES: Cc1cccc2c1N1c3ccccc3C(C)(C)c3cc(-c4cccc(N(c5ccc6c(c5)C(C)(C)c5ccccc5-6)c5ccccc5-c5ccccc5)c4)cc(c31)O2 ; Cc1cccc2c1N1c3ccccc3C(C)(C)c3cc(-c4cccc(N(c5ccc6c(c5)C(C)(C)c5ccccc5-6)c5cccc6ccccc56)c4)cc(c31)O2 ; Cc1cccc2c1N1c3ccccc3C(C)(C)c3cc(-c4cccc(N(c5ccc6ccccc6c5)c5cccc6ccccc56)c4)cc(c31)O2 ; Cc1cccc2c1N1c3ccccc3C(C)(C)c3cc(-c4cccc(N(c5ccc(-c6ccccc6)cc5)c5cc6ccccc6c6ccccc56)c4)cc(c31)O2]

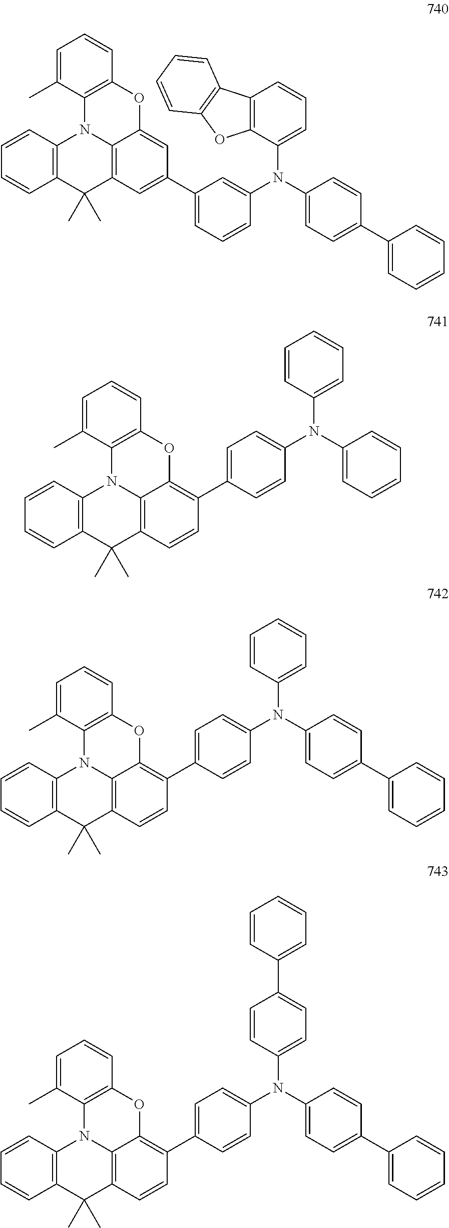 [SMILES: Cc1cccc2c1N1c3ccccc3C(C)(C)c3cc(-c4cccc(N(c5ccc(-c6ccccc6)cc5)c5cccc6c5oc5ccccc56)c4)cc(c31)O2 ; Cc1cccc2c1N1c3ccccc3C(C)(C)c3ccc(-c4ccc(N(c5ccccc5)c5ccccc5)cc4)c(c31)O2 ; Cc1cccc2c1N1c3ccccc3C(C)(C)c3ccc(-c4ccc(N(c5ccccc5)c5ccc(-c6ccccc6)cc5)cc4)c(c31)O2 ; Cc1cccc2c1N1c3ccccc3C(C)(C)c3ccc(-c4ccc(N(c5ccc(-c6ccccc6)cc5)c5ccc(-c6ccccc6)cc5)cc4)c(c31)O2]

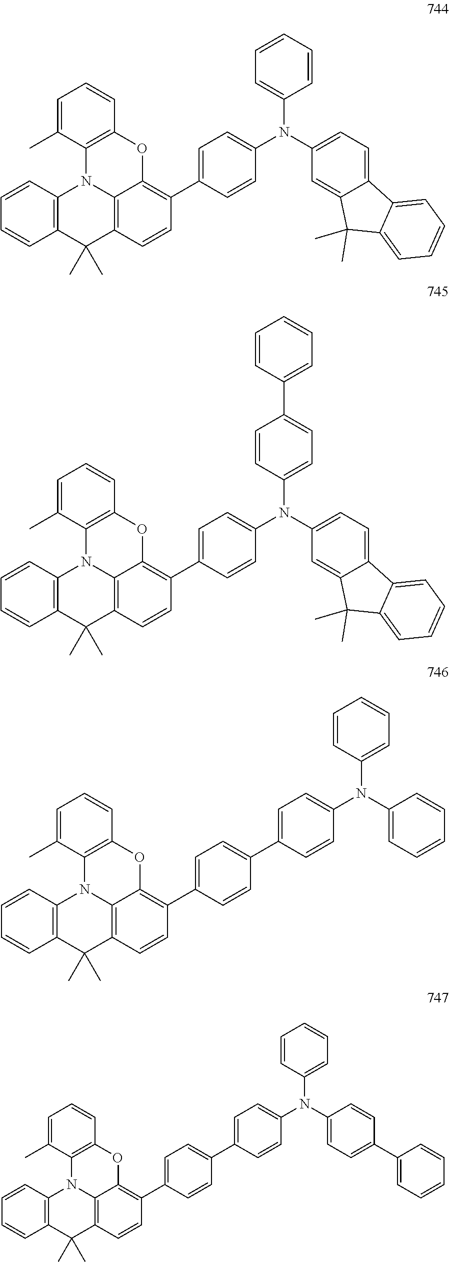 [SMILES: Cc1cccc2c1N1c3ccccc3C(C)(C)c3ccc(-c4ccc(N(c5ccccc5)c5ccc6c(c5)C(C)(C)c5ccccc5-6)cc4)c(c31)O2 ; Cc1cccc2c1N1c3ccccc3C(C)(C)c3ccc(-c4ccc(N(c5ccc(-c6ccccc6)cc5)c5ccc6c(c5)C(C)(C)c5ccccc5-6)cc4)c(c31)O2 ; Cc1cccc2c1N1c3ccccc3C(C)(C)c3ccc(-c4ccc(-c5ccc(N(c6ccccc6)c6ccccc6)cc5)cc4)c(c31)O2 ; Cc1cccc2c1N1c3ccccc3C(C)(C)c3ccc(-c4ccc(-c5ccc(N(c6ccccc6)c6ccc(-c7ccccc7)cc6)cc5)cc4)c(c31)O2]

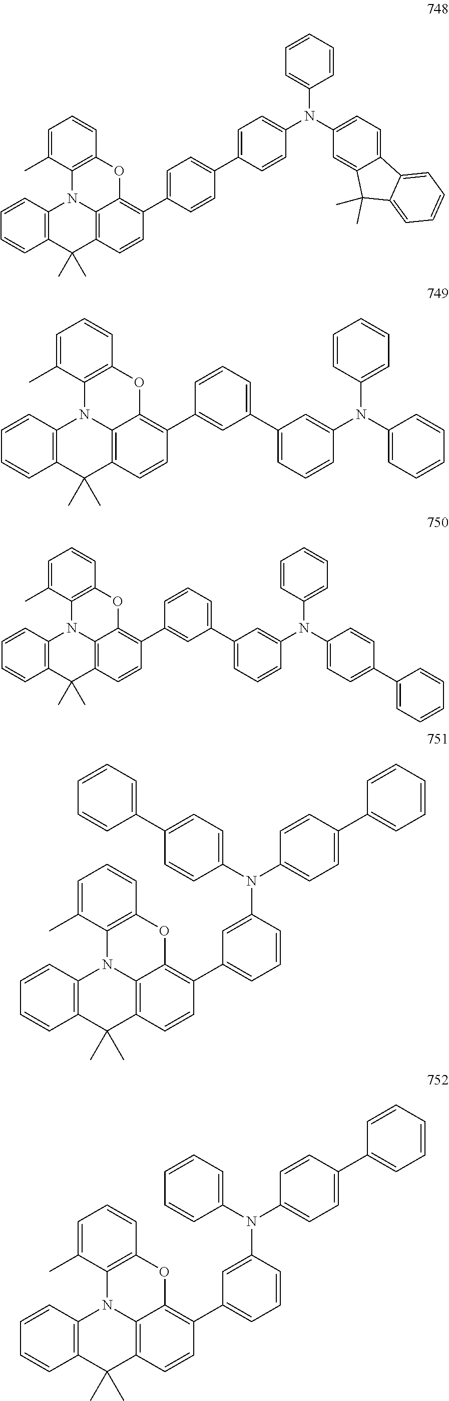

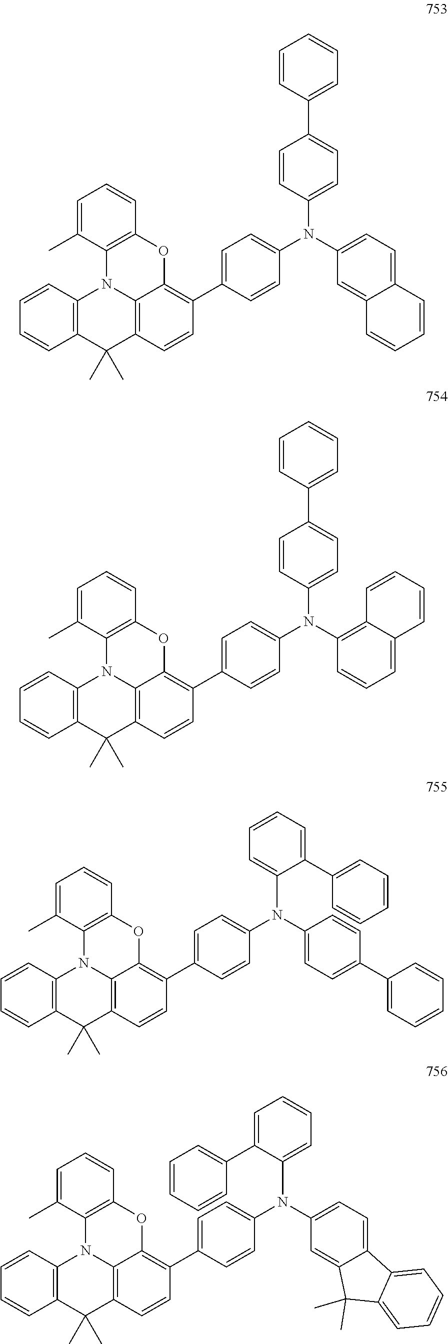 [SMILES: Cc1cccc2c1N1c3ccccc3C(C)(C)c3ccc(-c4ccc(N(c5ccc(-c6ccccc6)cc5)c5ccc6ccccc6c5)cc4)c(c31)O2 ; Cc1cccc2c1N1c3ccccc3C(C)(C)c3ccc(-c4ccc(N(c5ccc(-c6ccccc6)cc5)c5cccc6ccccc56)cc4)c(c31)O2 ; Cc1cccc2c1N1c3ccccc3C(C)(C)c3ccc(-c4ccc(N(c5ccc(-c6ccccc6)cc5)c5ccccc5-c5ccccc5)cc4)c(c31)O2 ; Cc1cccc2c1N1c3ccccc3C(C)(C)c3ccc(-c4ccc(N(c5ccc6c(c5)C(C)(C)c5ccccc5-6)c5ccccc5-c5ccccc5)cc4)c(c31)O2]

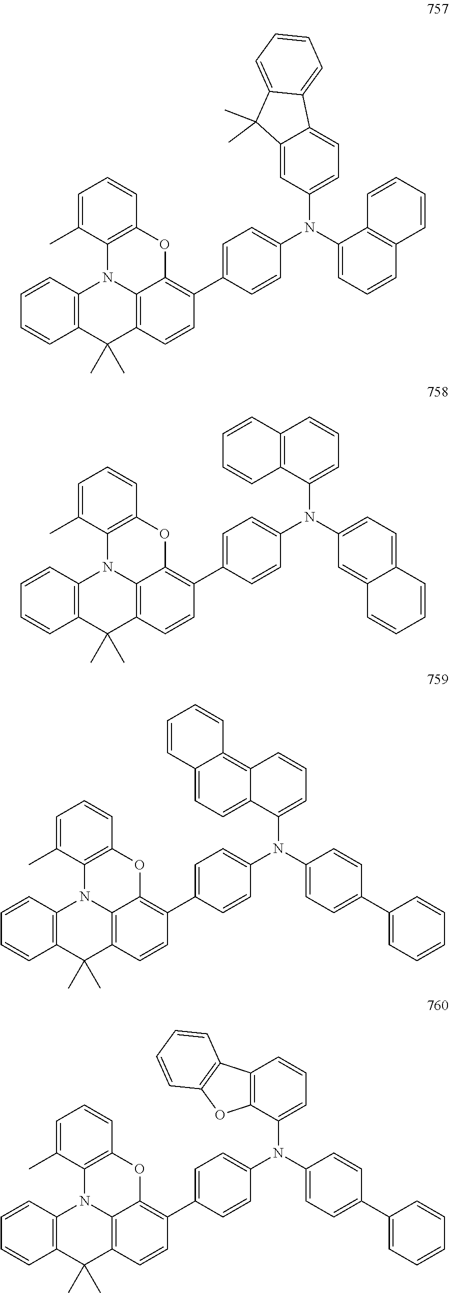 [SMILES: Cc1cccc2c1N1c3ccccc3C(C)(C)c3ccc(-c4ccc(N(c5ccc6c(c5)C(C)(C)c5ccccc5-6)c5cccc6ccccc56)cc4)c(c31)O2 ; Cc1cccc2c1N1c3ccccc3C(C)(C)c3ccc(-c4ccc(N(c5ccc6ccccc6c5)c5cccc6ccccc56)cc4)c(c31)O2 ; Cc1cccc2c1N1c3ccccc3C(C)(C)c3ccc(-c4ccc(N(c5ccc(-c6ccccc6)cc5)c5cccc6c5ccc5ccccc56)cc4)c(c31)O2 ; Cc1cccc2c1N1c3ccccc3C(C)(C)c3ccc(-c4ccc(N(c5ccc(-c6ccccc6)cc5)c5cccc6c5oc5ccccc56)cc4)c(c31)O2]

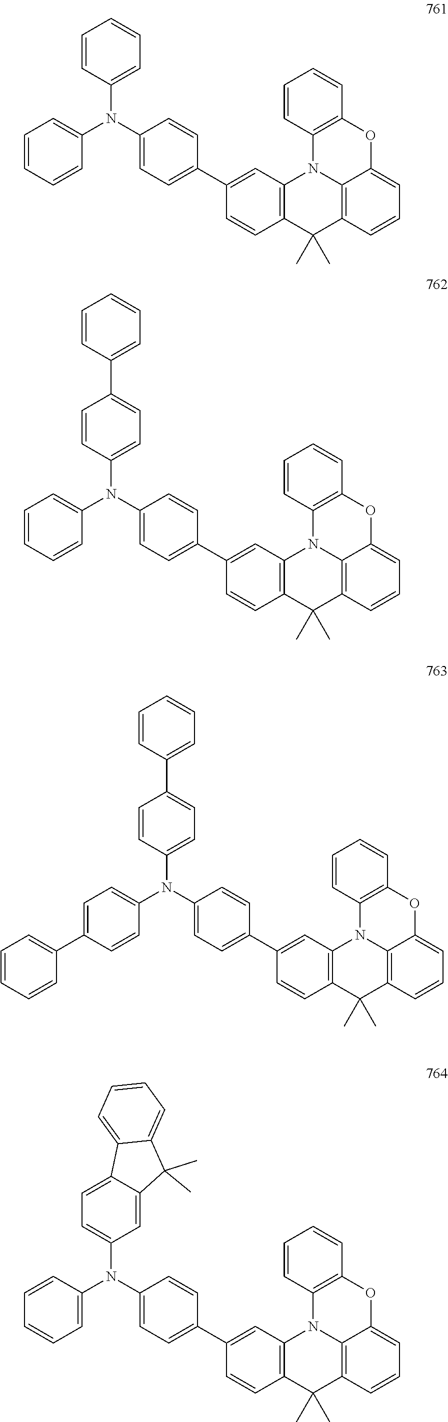 [SMILES: CC1(C)c2ccc(-c3ccc(N(c4ccccc4)c4ccccc4)cc3)cc2N2c3ccccc3Oc3cccc1c32 ; CC1(C)c2ccc(-c3ccc(N(c4ccccc4)c4ccc(-c5ccccc5)cc4)cc3)cc2N2c3ccccc3Oc3cccc1c32 ; CC1(C)c2ccc(-c3ccc(N(c4ccc(-c5ccccc5)cc4)c4ccc(-c5ccccc5)cc4)cc3)cc2N2c3ccccc3Oc3cccc1c32 ; CC1(C)c2ccccc2-c2ccc(N(c3ccccc3)c3ccc(-c4ccc5c(c4)N4c6ccccc6Oc6cccc(c64)C5(C)C)cc3)cc21]

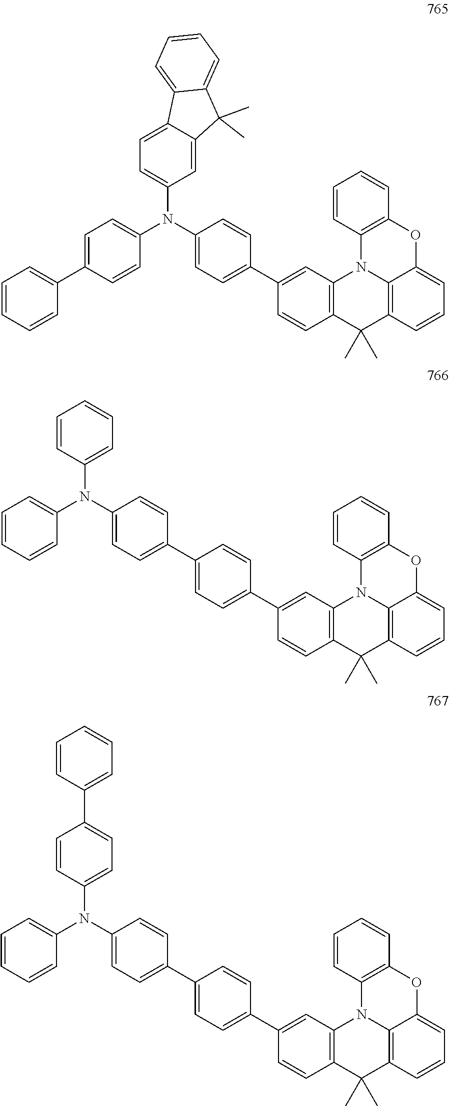 [SMILES: CC1(C)c2ccccc2-c2ccc(N(c3ccc(-c4ccccc4)cc3)c3ccc(-c4ccc5c(c4)N4c6ccccc6Oc6cccc(c64)C5(C)C)cc3)cc21 ; CC1(C)c2ccc(-c3ccc(-c4ccc(N(c5ccccc5)c5ccccc5)cc4)cc3)cc2N2c3ccccc3Oc3cccc1c32 ; CC1(C)c2ccc(-c3ccc(-c4ccc(N(c5ccccc5)c5ccc(-c6ccccc6)cc5)cc4)cc3)cc2N2c3ccccc3Oc3cccc1c32]

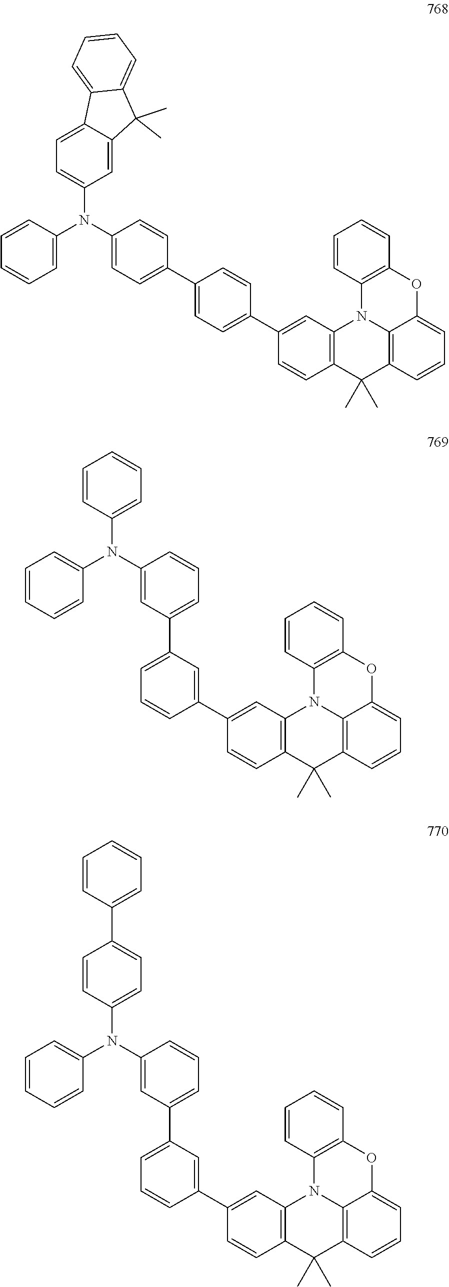 [SMILES: CC1(C)c2ccccc2-c2ccc(N(c3ccccc3)c3ccc(-c4ccc(-c5ccc6c(c5)N5c7ccccc7Oc7cccc(c75)C6(C)C)cc4)cc3)cc21 ; CC1(C)c2ccc(-c3cccc(-c4cccc(N(c5ccccc5)c5ccccc5)c4)c3)cc2N2c3ccccc3Oc3cccc1c32 ; CC1(C)c2ccc(-c3cccc(-c4cccc(N(c5ccccc5)c5ccc(-c6ccccc6)cc5)c4)c3)cc2N2c3ccccc3Oc3cccc1c32]

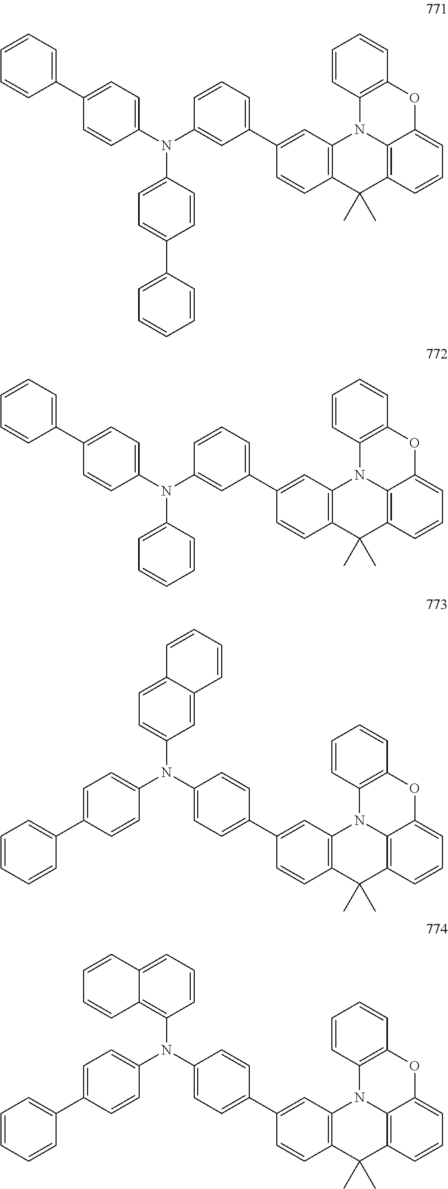 [SMILES: CC1(C)c2ccc(-c3cccc(N(c4ccc(-c5ccccc5)cc4)c4ccc(-c5ccccc5)cc4)c3)cc2N2c3ccccc3Oc3cccc1c32 ; CC1(C)c2ccc(-c3cccc(N(c4ccccc4)c4ccc(-c5ccccc5)cc4)c3)cc2N2c3ccccc3Oc3cccc1c32 ; CC1(C)c2ccc(-c3ccc(N(c4ccc(-c5ccccc5)cc4)c4ccc5ccccc5c4)cc3)cc2N2c3ccccc3Oc3cccc1c32 ; CC1(C)c2ccc(-c3ccc(N(c4ccc(-c5ccccc5)cc4)c4cccc5ccccc45)cc3)cc2N2c3ccccc3Oc3cccc1c32]

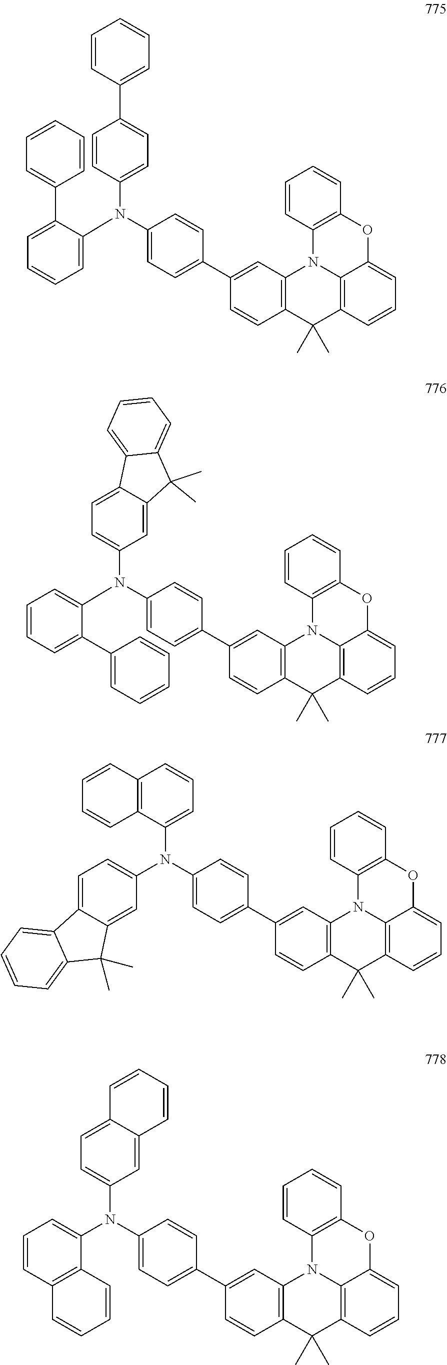 [SMILES: CC1(C)c2ccc(-c3ccc(N(c4ccc(-c5ccccc5)cc4)c4ccccc4-c4ccccc4)cc3)cc2N2c3ccccc3Oc3cccc1c32 ; CC1(C)c2ccccc2-c2ccc(N(c3ccc(-c4ccc5c(c4)N4c6ccccc6Oc6cccc(c64)C5(C)C)cc3)c3ccccc3-c3ccccc3)cc21 ; CC1(C)c2ccccc2-c2ccc(N(c3ccc(-c4ccc5c(c4)N4c6ccccc6Oc6cccc(c64)C5(C)C)cc3)c3cccc4ccccc34)cc21 ; CC1(C)c2ccc(-c3ccc(N(c4ccc5ccccc5c4)c4cccc5ccccc45)cc3)cc2N2c3ccccc3Oc3cccc1c32]

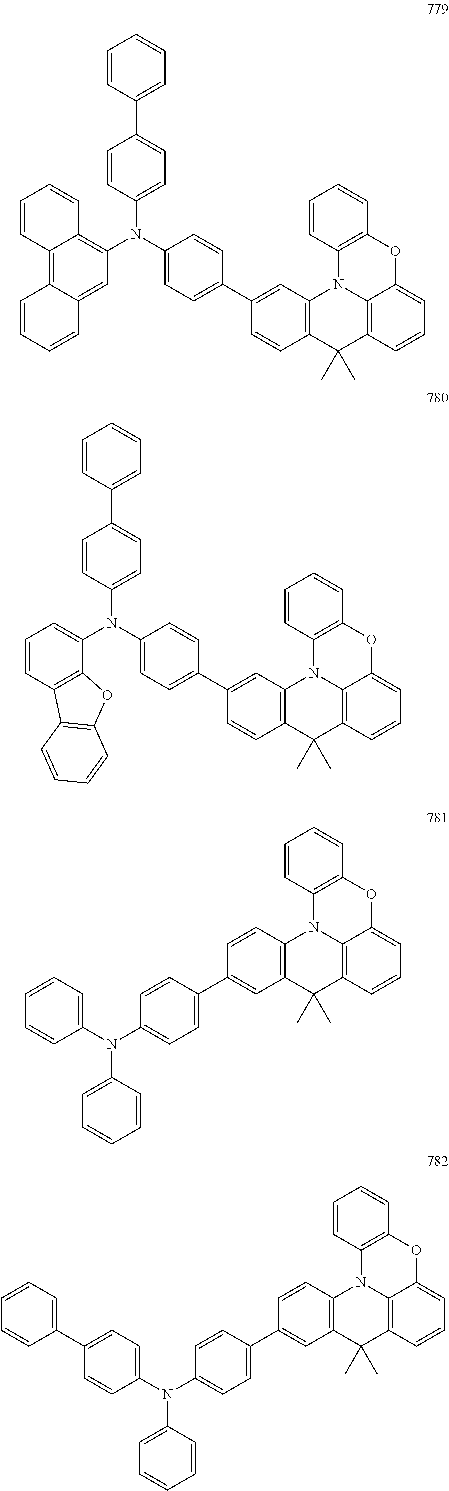 [SMILES: CC1(C)c2ccc(-c3ccc(N(c4ccc(-c5ccccc5)cc4)c4cc5ccccc5c5ccccc45)cc3)cc2N2c3ccccc3Oc3cccc1c32 ; CC1(C)c2ccc(-c3ccc(N(c4ccc(-c5ccccc5)cc4)c4cccc5c4oc4ccccc45)cc3)cc2N2c3ccccc3Oc3cccc1c32 ; CC1(C)c2cc(-c3ccc(N(c4ccccc4)c4ccccc4)cc3)ccc2N2c3ccccc3Oc3cccc1c32 ; CC1(C)c2cc(-c3ccc(N(c4ccccc4)c4ccc(-c5ccccc5)cc4)cc3)ccc2N2c3ccccc3Oc3cccc1c32]

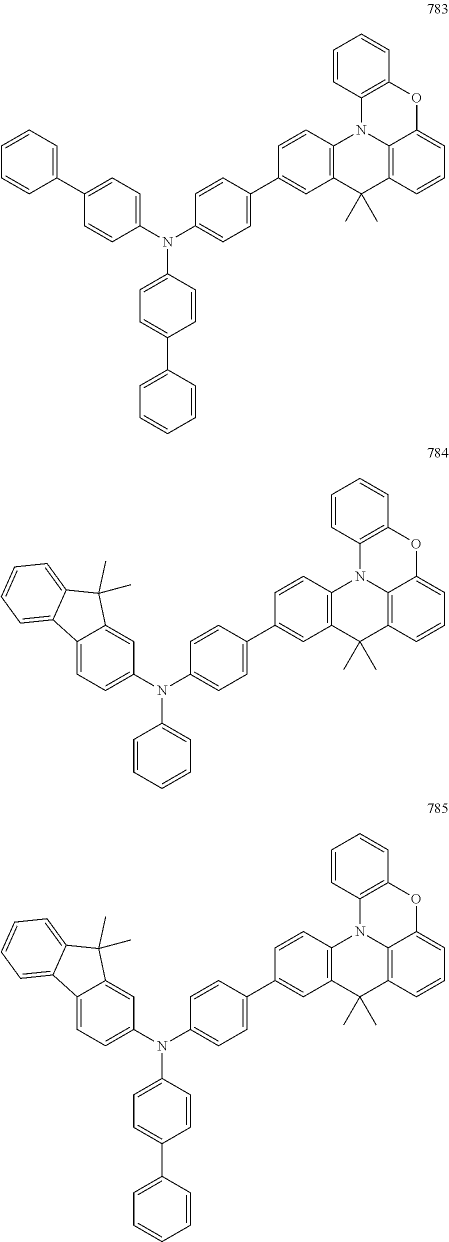 [SMILES: CC1(C)c2cc(-c3ccc(N(c4ccc(-c5ccccc5)cc4)c4ccc(-c5ccccc5)cc4)cc3)ccc2N2c3ccccc3Oc3cccc1c32 ; CC1(C)c2ccccc2-c2ccc(N(c3ccccc3)c3ccc(-c4ccc5c(c4)C(C)(C)c4cccc6c4N5c4ccccc4O6)cc3)cc21 ; CC1(C)c2ccccc2-c2ccc(N(c3ccc(-c4ccccc4)cc3)c3ccc(-c4ccc5c(c4)C(C)(C)c4cccc6c4N5c4ccccc4O6)cc3)cc21]

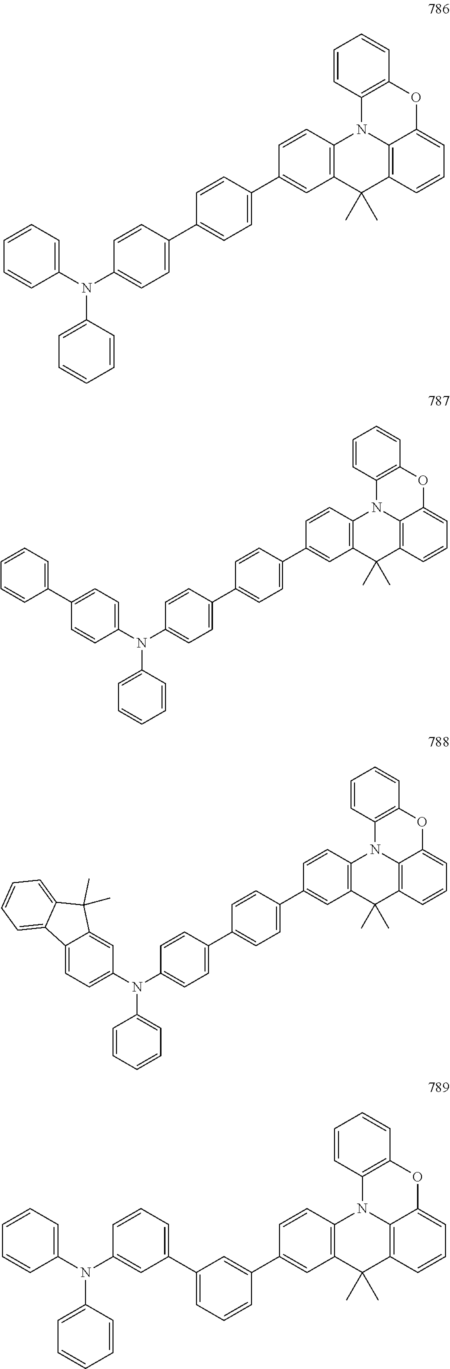 [SMILES: CC1(C)c2cc(-c3ccc(-c4ccc(N(c5ccccc5)c5ccccc5)cc4)cc3)ccc2N2c3ccccc3Oc3cccc1c32 ; CC1(C)c2cc(-c3ccc(-c4ccc(N(c5ccccc5)c5ccc(-c6ccccc6)cc5)cc4)cc3)ccc2N2c3ccccc3Oc3cccc1c32 ; CC1(C)c2ccccc2-c2ccc(N(c3ccccc3)c3ccc(-c4ccc(-c5ccc6c(c5)C(C)(C)c5cccc7c5N6c5ccccc5O7)cc4)cc3)cc21 ; CC1(C)c2cc(-c3cccc(-c4cccc(N(c5ccccc5)c5ccccc5)c4)c3)ccc2N2c3ccccc3Oc3cccc1c32]

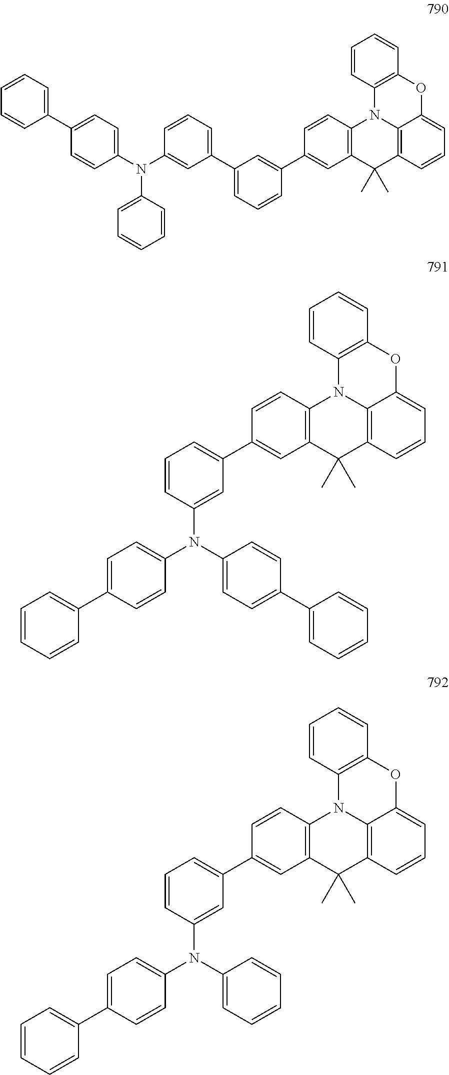 [SMILES: CC1(C)c2cc(-c3cccc(-c4cccc(N(c5ccccc5)c5ccc(-c6ccccc6)cc5)c4)c3)ccc2N2c3ccccc3Oc3cccc1c32 ; CC1(C)c2cc(-c3cccc(N(c4ccc(-c5ccccc5)cc4)c4ccc(-c5ccccc5)cc4)c3)ccc2N2c3ccccc3Oc3cccc1c32 ; CC1(C)c2cc(-c3cccc(N(c4ccccc4)c4ccc(-c5ccccc5)cc4)c3)ccc2N2c3ccccc3Oc3cccc1c32]

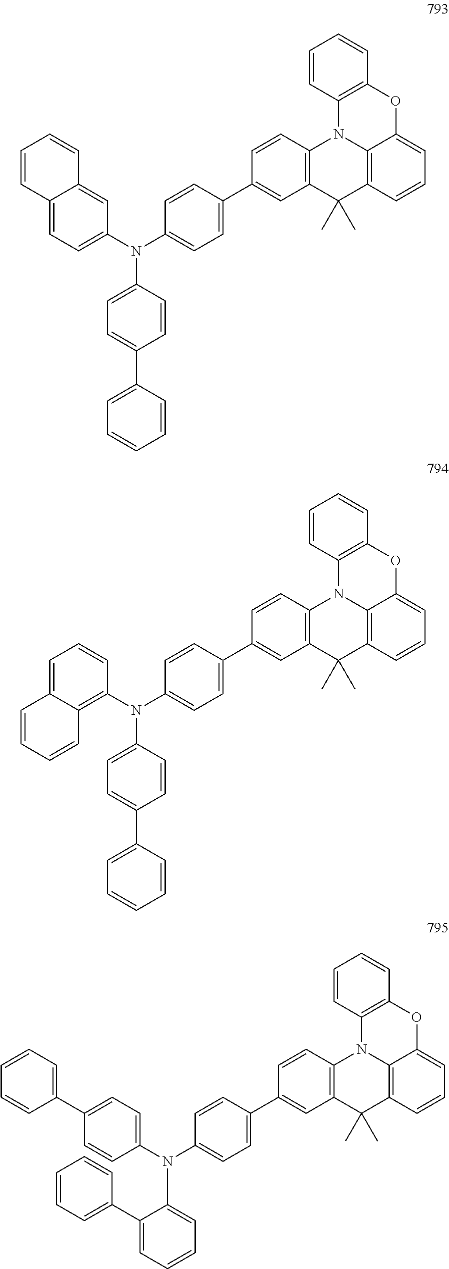 [SMILES: CC1(C)c2cc(-c3ccc(N(c4ccc(-c5ccccc5)cc4)c4ccc5ccccc5c4)cc3)ccc2N2c3ccccc3Oc3cccc1c32 ; CC1(C)c2cc(-c3ccc(N(c4ccc(-c5ccccc5)cc4)c4cccc5ccccc45)cc3)ccc2N2c3ccccc3Oc3cccc1c32 ; CC1(C)c2cc(-c3ccc(N(c4ccc(-c5ccccc5)cc4)c4ccccc4-c4ccccc4)cc3)ccc2N2c3ccccc3Oc3cccc1c32]

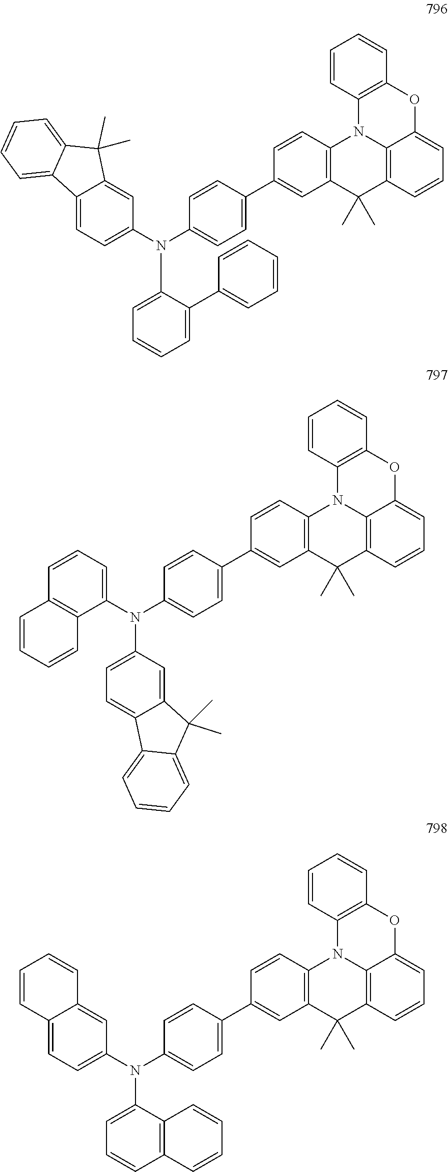 [SMILES: CC1(C)c2ccccc2-c2ccc(N(c3ccc(-c4ccc5c(c4)C(C)(C)c4cccc6c4N5c4ccccc4O6)cc3)c3ccccc3-c3ccccc3)cc21 ; CC1(C)c2ccccc2-c2ccc(N(c3ccc(-c4ccc5c(c4)C(C)(C)c4cccc6c4N5c4ccccc4O6)cc3)c3cccc4ccccc34)cc21 ; CC1(C)c2cc(-c3ccc(N(c4ccc5ccccc5c4)c4cccc5ccccc45)cc3)ccc2N2c3ccccc3Oc3cccc1c32]

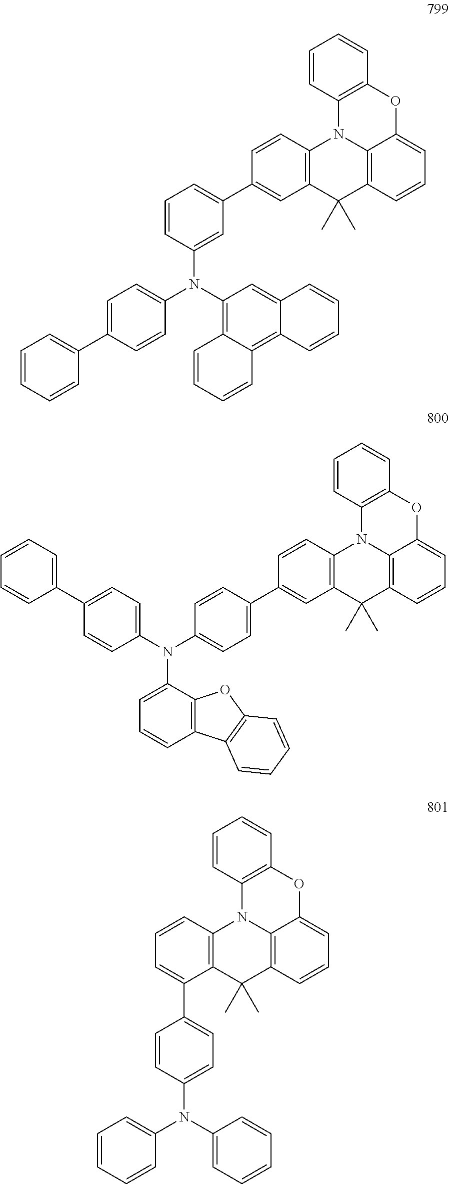

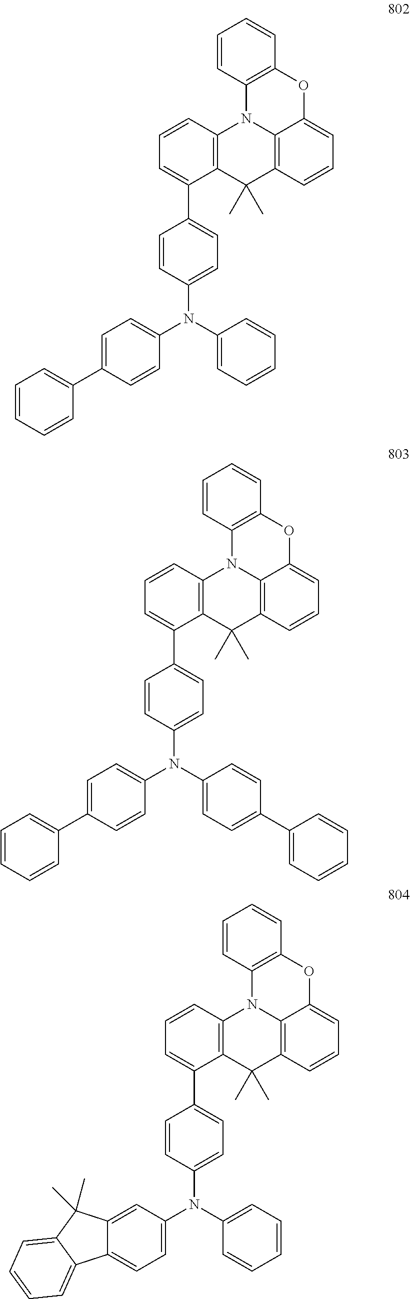 [SMILES: CC1(C)c2cccc3c2N(c2ccccc2O3)c2cccc(-c3ccc(N(c4ccccc4)c4ccc(-c5ccccc5)cc4)cc3)c21 ; CC1(C)c2cccc3c2N(c2ccccc2O3)c2cccc(-c3ccc(N(c4ccc(-c5ccccc5)cc4)c4ccc(-c5ccccc5)cc4)cc3)c21 ; CC1(C)c2ccccc2-c2ccc(N(c3ccccc3)c3ccc(-c4cccc5c4C(C)(C)c4cccc6c4N5c4ccccc4O6)cc3)cc21]

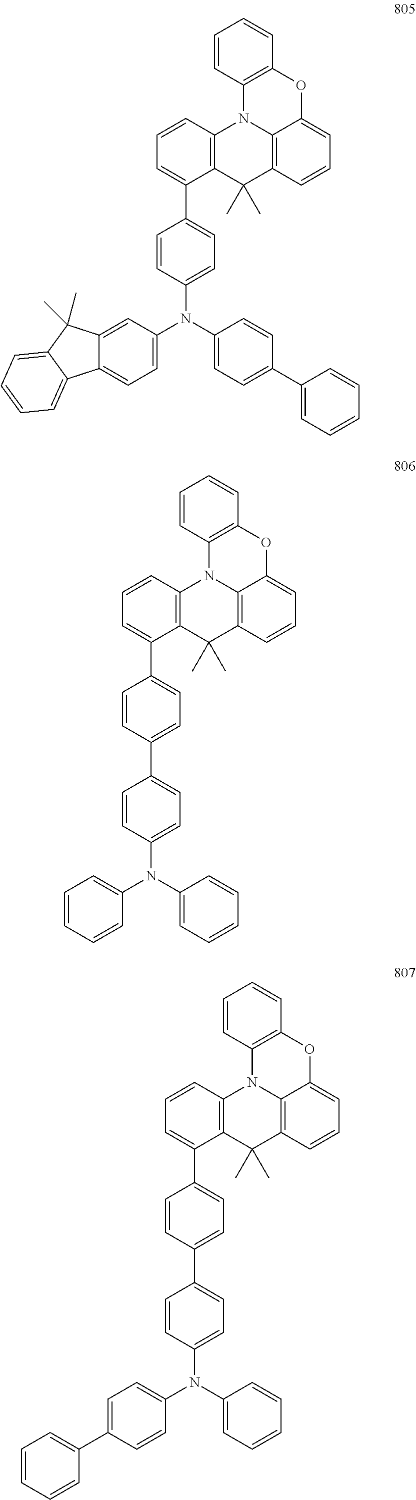 [SMILES: CC1(C)c2ccccc2-c2ccc(N(c3ccc(-c4ccccc4)cc3)c3ccc(-c4cccc5c4C(C)(C)c4cccc6c4N5c4ccccc4O6)cc3)cc21 ; CC1(C)c2cccc3c2N(c2ccccc2O3)c2cccc(-c3ccc(-c4ccc(N(c5ccccc5)c5ccccc5)cc4)cc3)c21 ; CC1(C)c2cccc3c2N(c2ccccc2O3)c2cccc(-c3ccc(-c4ccc(N(c5ccccc5)c5ccc(-c6ccccc6)cc5)cc4)cc3)c21]

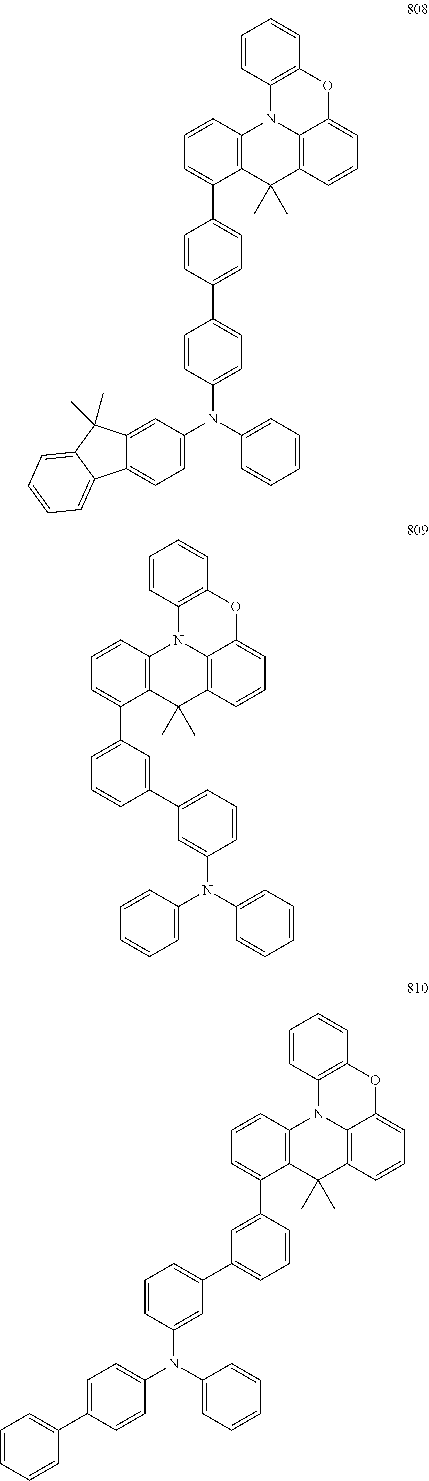 [SMILES: CC1(C)c2ccccc2-c2ccc(N(c3ccccc3)c3ccc(-c4ccc(-c5cccc6c5C(C)(C)c5cccc7c5N6c5ccccc5O7)cc4)cc3)cc21 ; CC1(C)c2cccc3c2N(c2ccccc2O3)c2cccc(-c3cccc(-c4cccc(N(c5ccccc5)c5ccccc5)c4)c3)c21 ; CC1(C)c2cccc3c2N(c2ccccc2O3)c2cccc(-c3cccc(-c4cccc(N(c5ccccc5)c5ccc(-c6ccccc6)cc5)c4)c3)c21]

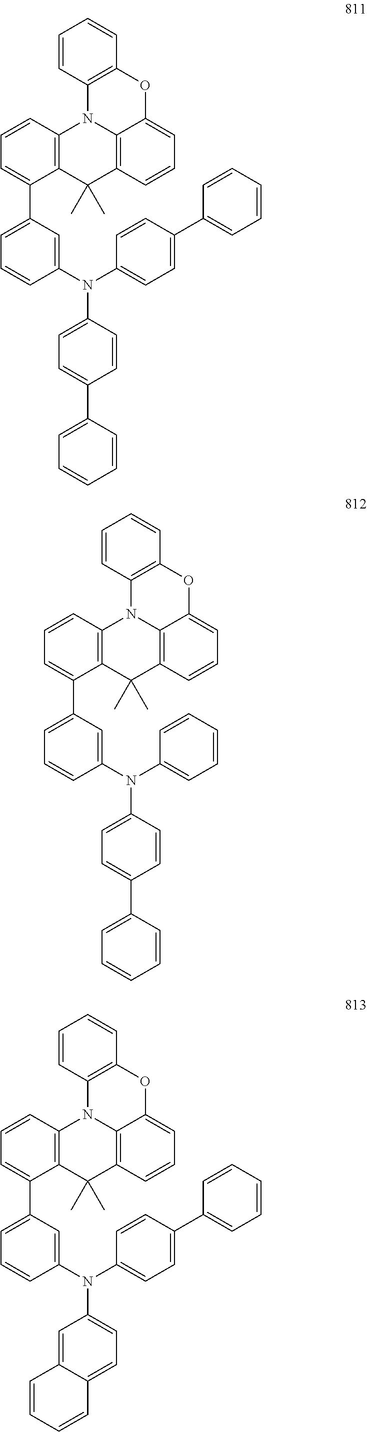 [SMILES: CC1(C)c2cccc3c2N(c2ccccc2O3)c2cccc(-c3cccc(N(c4ccc(-c5ccccc5)cc4)c4ccc(-c5ccccc5)cc4)c3)c21 ; CC1(C)c2cccc3c2N(c2ccccc2O3)c2cccc(-c3cccc(N(c4ccccc4)c4ccc(-c5ccccc5)cc4)c3)c21 ; CC1(C)c2cccc3c2N(c2ccccc2O3)c2cccc(-c3cccc(N(c4ccc(-c5ccccc5)cc4)c4ccc5ccccc5c4)c3)c21]

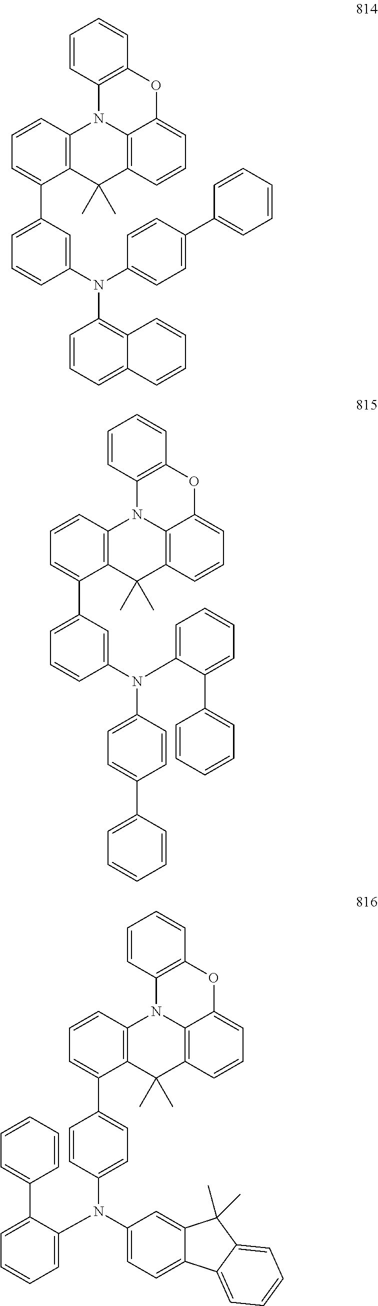

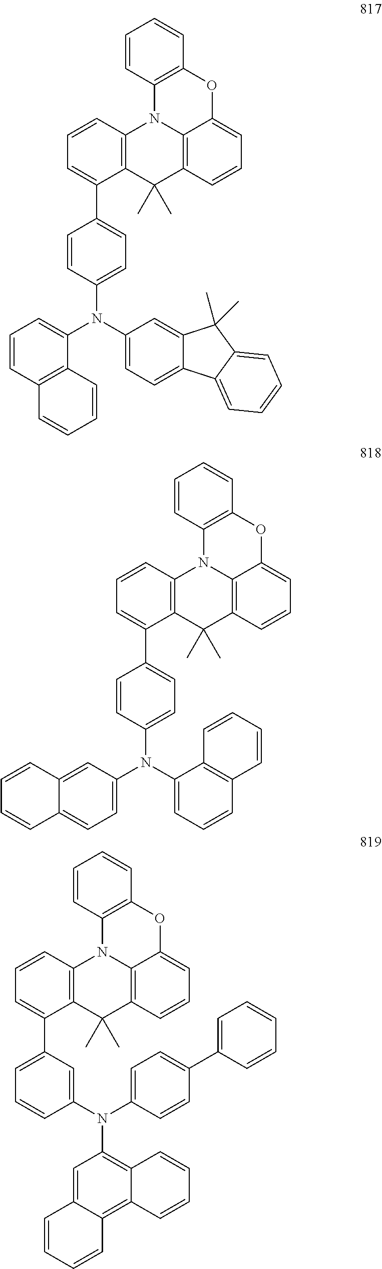

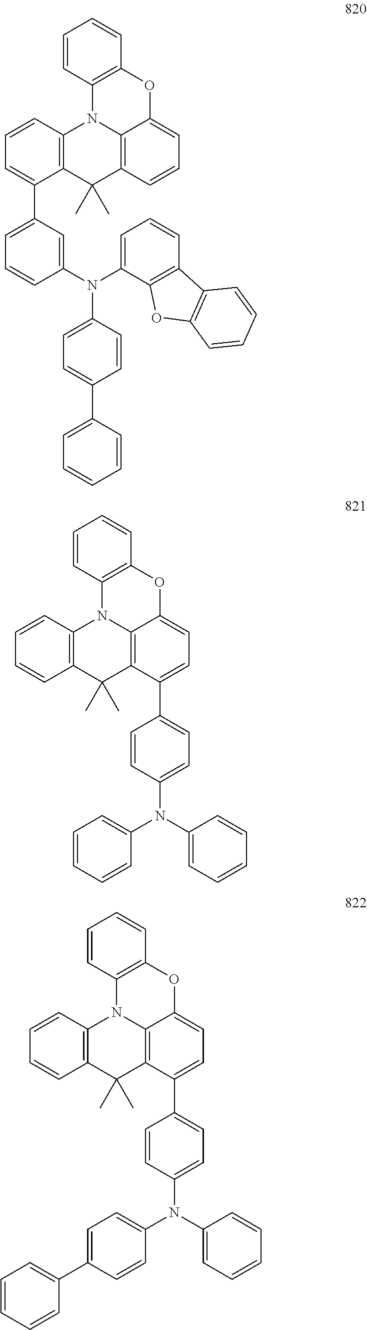

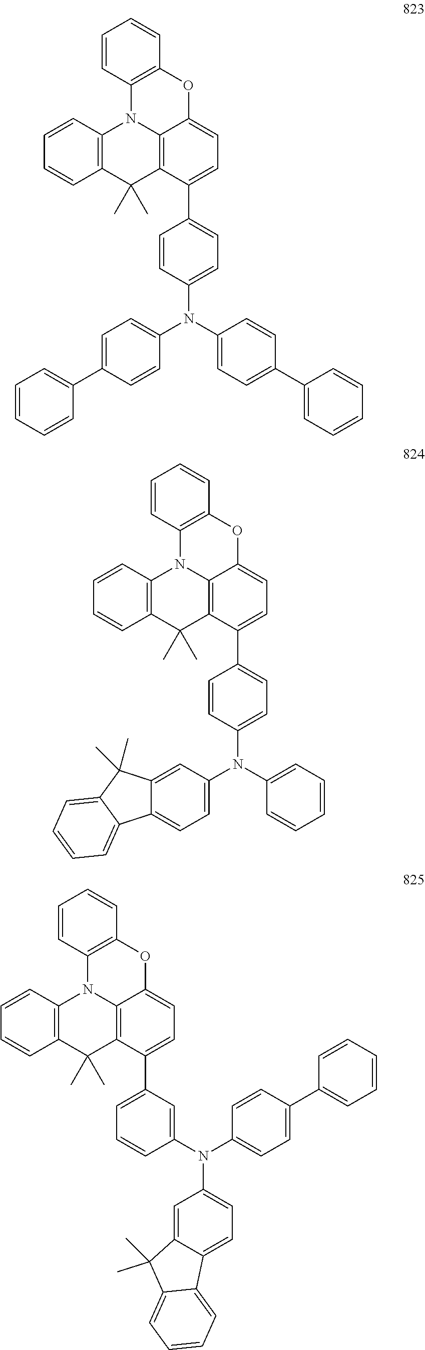

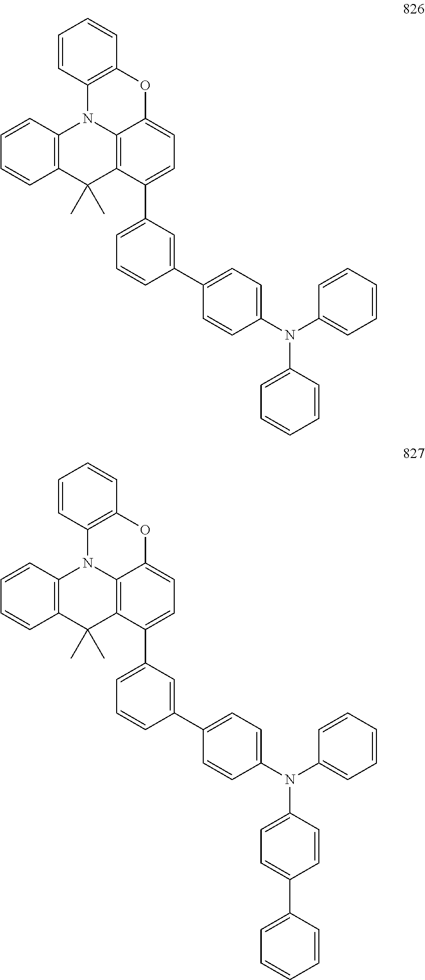 [SMILES: CC1(C)c2ccccc2N2c3ccccc3Oc3ccc(-c4cccc(-c5ccc(N(c6ccccc6)c6ccccc6)cc5)c4)c1c32 ; CC1(C)c2ccccc2N2c3ccccc3Oc3ccc(-c4cccc(-c5ccc(N(c6ccccc6)c6ccc(-c7ccccc7)cc6)cc5)c4)c1c32]

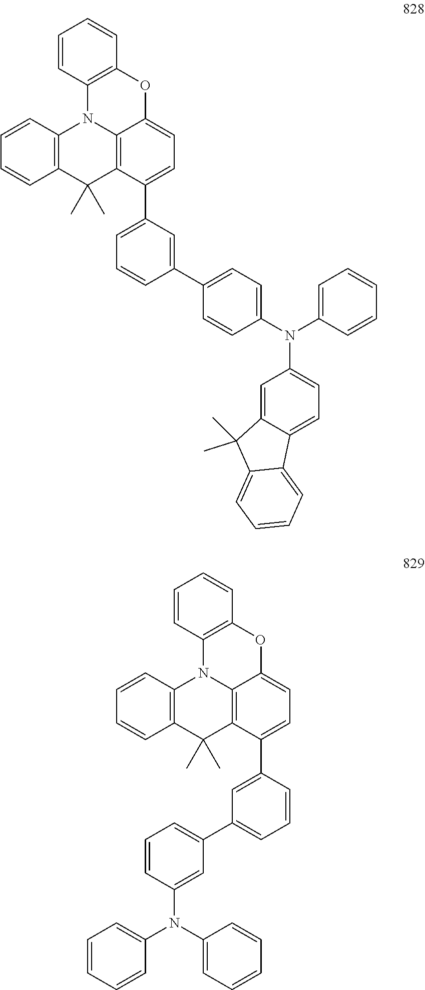 [SMILES: CC1(C)c2ccccc2-c2ccc(N(c3ccccc3)c3ccc(-c4cccc(-c5ccc6c7c5C(C)(C)c5ccccc5N7c5ccccc5O6)c4)cc3)cc21 ; CC1(C)c2ccccc2N2c3ccccc3Oc3ccc(-c4cccc(-c5cccc(N(c6ccccc6)c6ccccc6)c5)c4)c1c32]

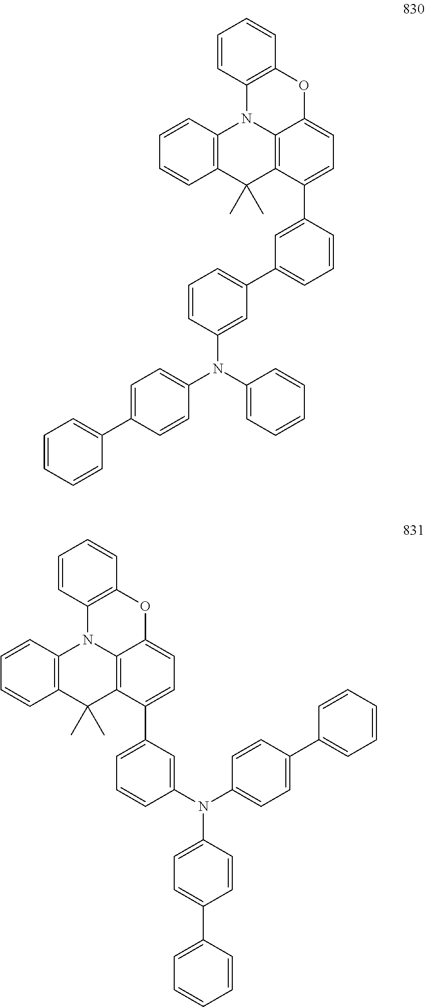 [SMILES: CC1(C)c2ccccc2N2c3ccccc3Oc3ccc(-c4cccc(-c5cccc(N(c6ccccc6)c6ccc(-c7ccccc7)cc6)c5)c4)c1c32 ; CC1(C)c2ccccc2N2c3ccccc3Oc3ccc(-c4cccc(N(c5ccc(-c6ccccc6)cc5)c5ccc(-c6ccccc6)cc5)c4)c1c32]

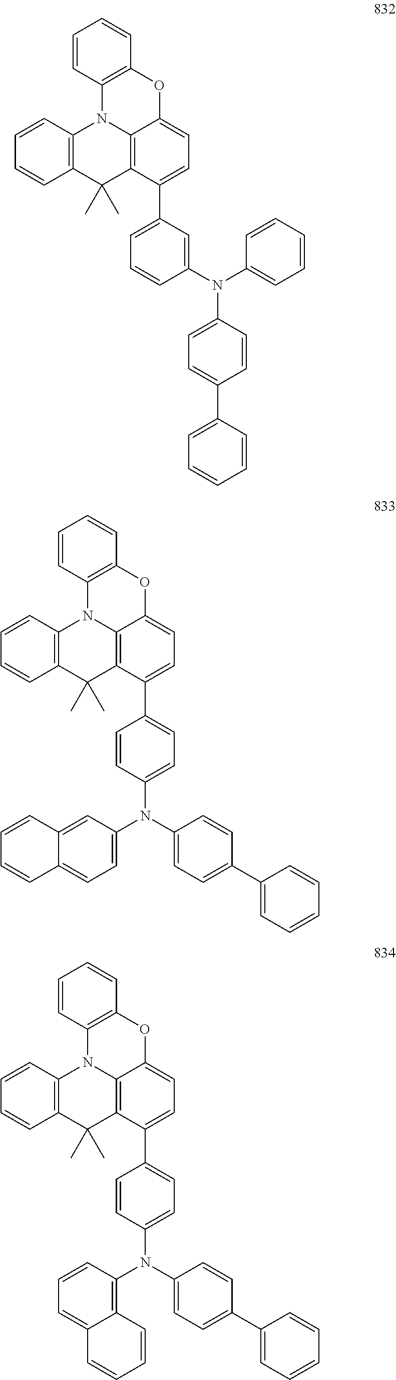 [SMILES: CC1(C)c2ccccc2N2c3ccccc3Oc3ccc(-c4cccc(N(c5ccccc5)c5ccc(-c6ccccc6)cc5)c4)c1c32 ; CC1(C)c2ccccc2N2c3ccccc3Oc3ccc(-c4ccc(N(c5ccc(-c6ccccc6)cc5)c5ccc6ccccc6c5)cc4)c1c32 ; CC1(C)c2ccccc2N2c3ccccc3Oc3ccc(-c4ccc(N(c5ccc(-c6ccccc6)cc5)c5cccc6ccccc56)cc4)c1c32]

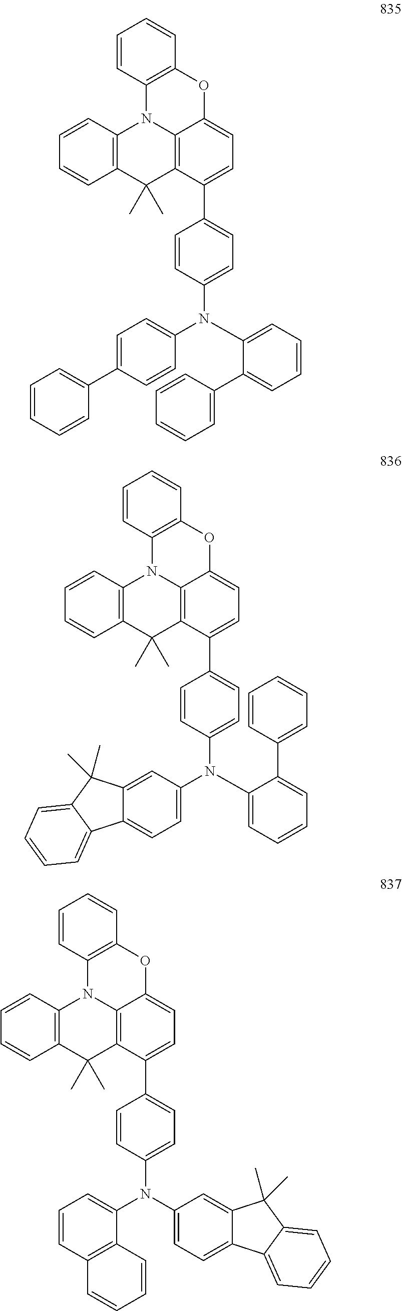

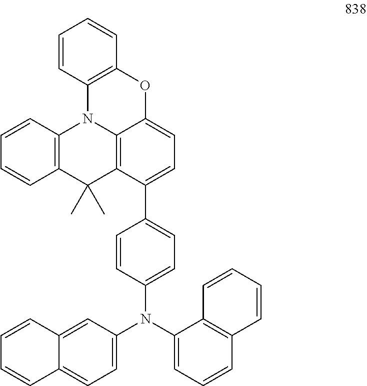 [SMILES: CC1(C)c2ccccc2N2c3ccccc3Oc3ccc(-c4ccc(N(c5ccc6ccccc6c5)c5cccc6ccccc56)cc4)c1c32]

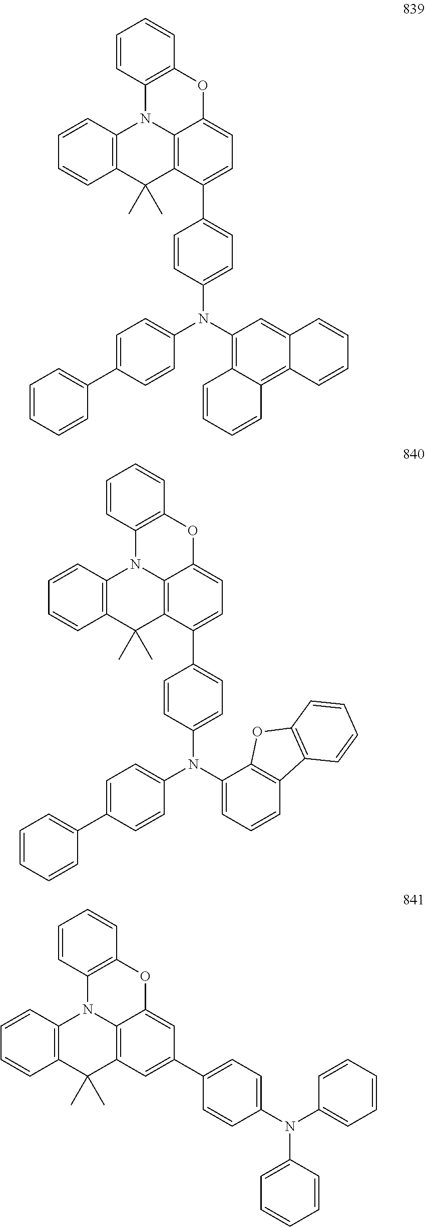

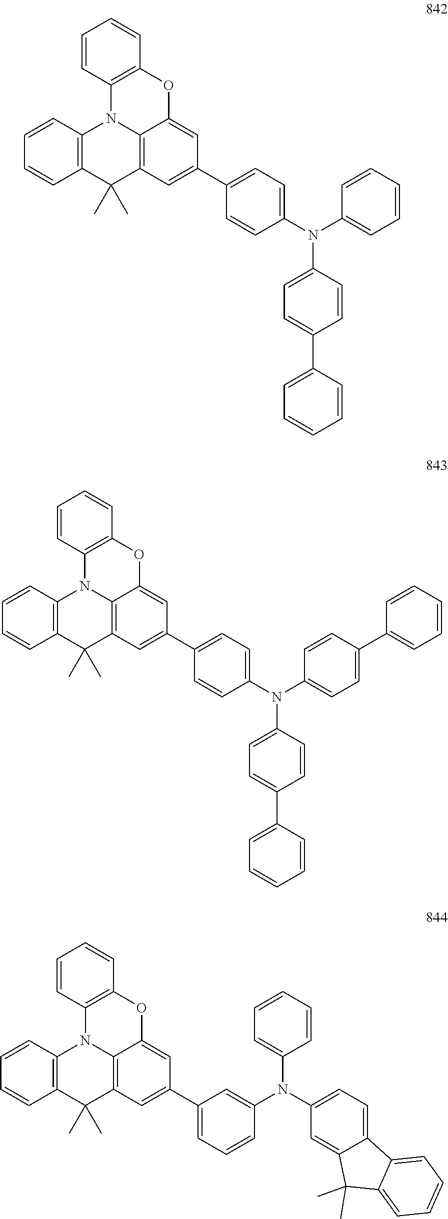 [SMILES: CC1(C)c2ccccc2N2c3ccccc3Oc3cc(-c4ccc(N(c5ccccc5)c5ccc(-c6ccccc6)cc5)cc4)cc1c32 ; CC1(C)c2ccccc2N2c3ccccc3Oc3cc(-c4ccc(N(c5ccc(-c6ccccc6)cc5)c5ccc(-c6ccccc6)cc5)cc4)cc1c32 ; CC1(C)c2ccccc2-c2ccc(N(c3ccccc3)c3cccc(-c4cc5c6c(c4)C(C)(C)c4ccccc4N6c4ccccc4O5)c3)cc21]

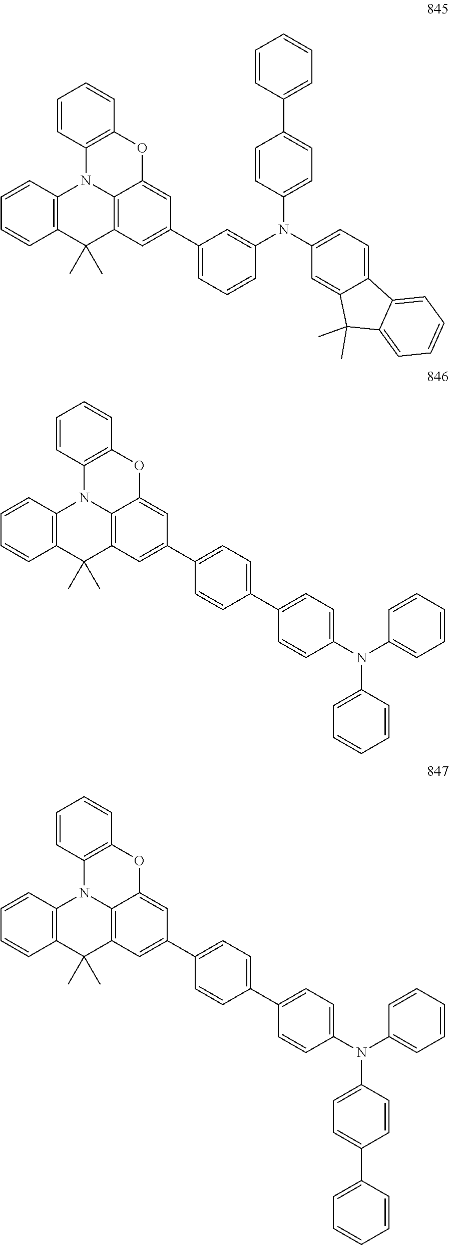 [SMILES: CC1(C)c2ccccc2-c2ccc(N(c3ccc(-c4ccccc4)cc3)c3cccc(-c4cc5c6c(c4)C(C)(C)c4ccccc4N6c4ccccc4O5)c3)cc21 ; CC1(C)c2ccccc2N2c3ccccc3Oc3cc(-c4ccc(-c5ccc(N(c6ccccc6)c6ccccc6)cc5)cc4)cc1c32 ; CC1(C)c2ccccc2N2c3ccccc3Oc3cc(-c4ccc(-c5ccc(N(c6ccccc6)c6ccc(-c7ccccc7)cc6)cc5)cc4)cc1c32]

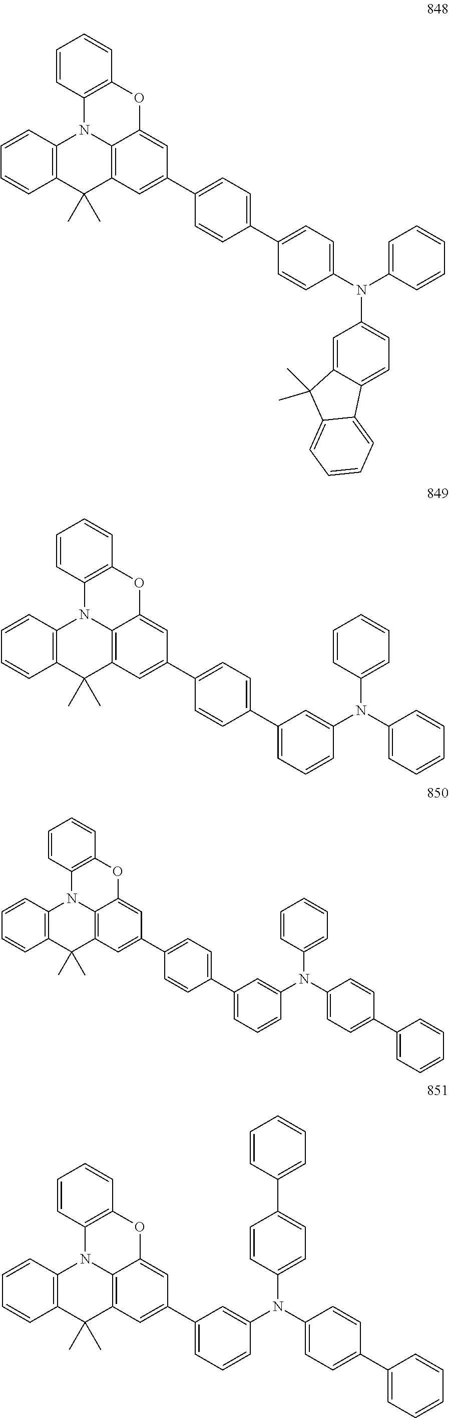 [SMILES: CC1(C)c2ccccc2-c2ccc(N(c3ccccc3)c3ccc(-c4ccc(-c5cc6c7c(c5)C(C)(C)c5ccccc5N7c5ccccc5O6)cc4)cc3)cc21 ; CC1(C)c2ccccc2N2c3ccccc3Oc3cc(-c4ccc(-c5cccc(N(c6ccccc6)c6ccccc6)c5)cc4)cc1c32 ; CC1(C)c2ccccc2N2c3ccccc3Oc3cc(-c4ccc(-c5cccc(N(c6ccccc6)c6ccc(-c7ccccc7)cc6)c5)cc4)cc1c32 ; CC1(C)c2ccccc2N2c3ccccc3Oc3cc(-c4cccc(N(c5ccc(-c6ccccc6)cc5)c5ccc(-c6ccccc6)cc5)c4)cc1c32]

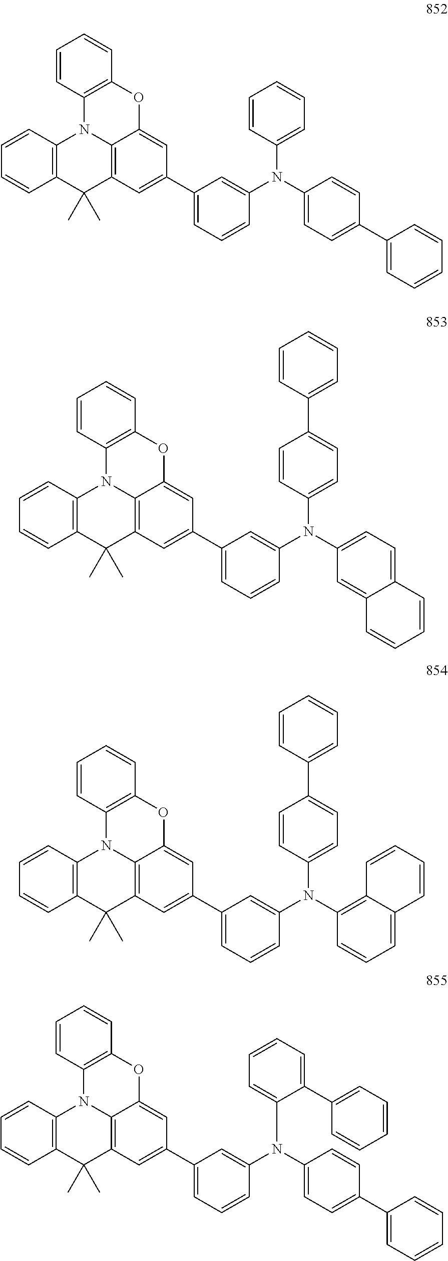 [SMILES: CC1(C)c2ccccc2N2c3ccccc3Oc3cc(-c4cccc(N(c5ccccc5)c5ccc(-c6ccccc6)cc5)c4)cc1c32 ; CC1(C)c2ccccc2N2c3ccccc3Oc3cc(-c4cccc(N(c5ccc(-c6ccccc6)cc5)c5ccc6ccccc6c5)c4)cc1c32 ; CC1(C)c2ccccc2N2c3ccccc3Oc3cc(-c4cccc(N(c5ccc(-c6ccccc6)cc5)c5cccc6ccccc56)c4)cc1c32 ; CC1(C)c2ccccc2N2c3ccccc3Oc3cc(-c4cccc(N(c5ccc(-c6ccccc6)cc5)c5ccccc5-c5ccccc5)c4)cc1c32]

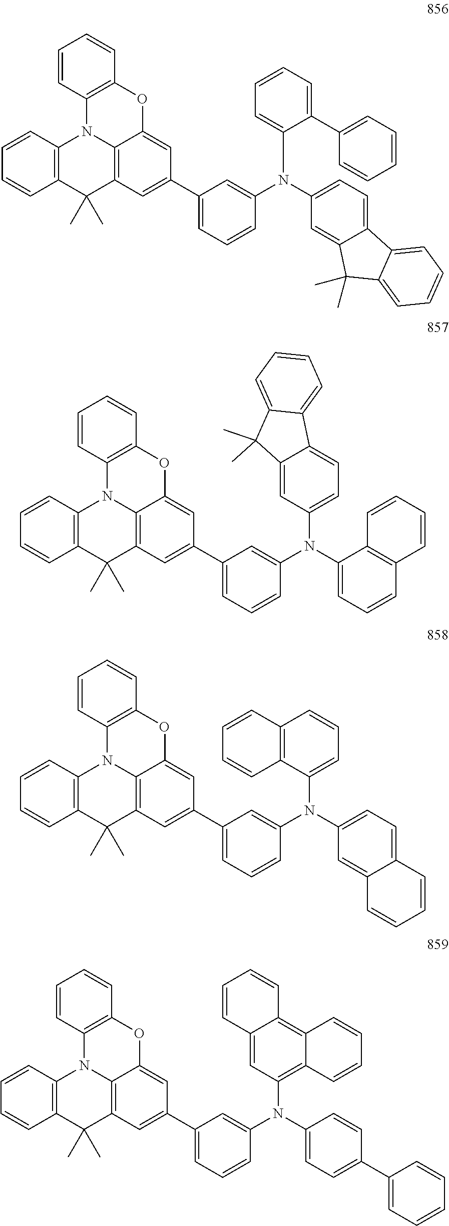 [SMILES: CC1(C)c2ccccc2-c2ccc(N(c3cccc(-c4cc5c6c(c4)C(C)(C)c4ccccc4N6c4ccccc4O5)c3)c3ccccc3-c3ccccc3)cc21 ; CC1(C)c2ccccc2-c2ccc(N(c3cccc(-c4cc5c6c(c4)C(C)(C)c4ccccc4N6c4ccccc4O5)c3)c3cccc4ccccc34)cc21 ; CC1(C)c2ccccc2N2c3ccccc3Oc3cc(-c4cccc(N(c5ccc6ccccc6c5)c5cccc6ccccc56)c4)cc1c32 ; CC1(C)c2ccccc2N2c3ccccc3Oc3cc(-c4cccc(N(c5ccc(-c6ccccc6)cc5)c5cc6ccccc6c6ccccc56)c4)cc1c32]

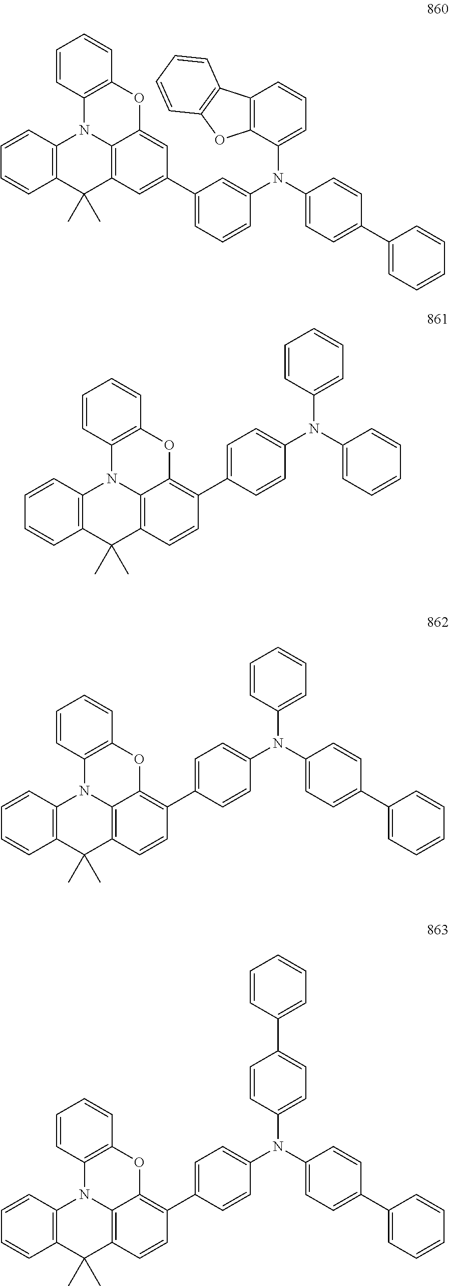 [SMILES: CC1(C)c2ccccc2N2c3ccccc3Oc3cc(-c4cccc(N(c5ccc(-c6ccccc6)cc5)c5cccc6c5oc5ccccc56)c4)cc1c32 ; CC1(C)c2ccccc2N2c3ccccc3Oc3c(-c4ccc(N(c5ccccc5)c5ccccc5)cc4)ccc1c32 ; CC1(C)c2ccccc2N2c3ccccc3Oc3c(-c4ccc(N(c5ccccc5)c5ccc(-c6ccccc6)cc5)cc4)ccc1c32 ; CC1(C)c2ccccc2N2c3ccccc3Oc3c(-c4ccc(N(c5ccc(-c6ccccc6)cc5)c5ccc(-c6ccccc6)cc5)cc4)ccc1c32]

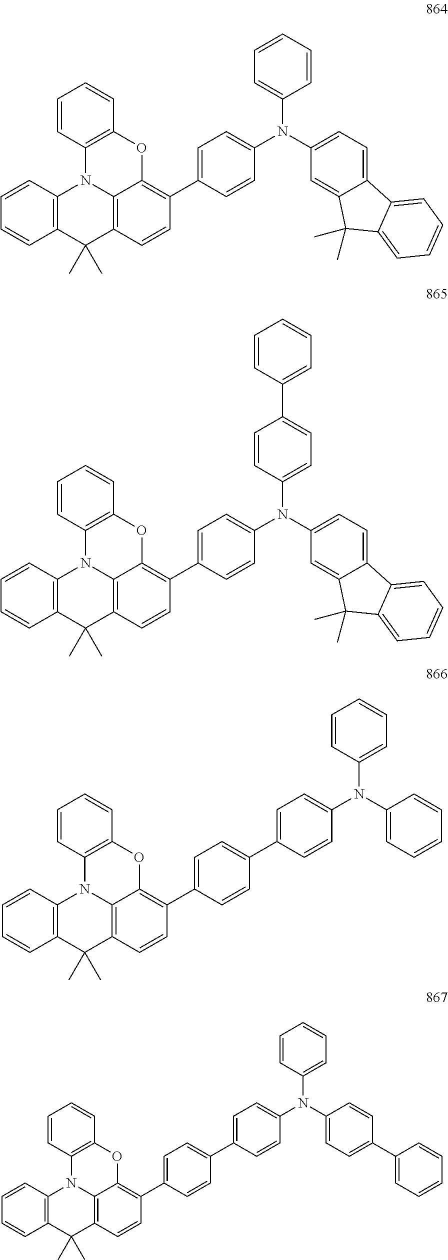 [SMILES: CC1(C)c2ccccc2-c2ccc(N(c3ccccc3)c3ccc(-c4ccc5c6c4Oc4ccccc4N6c4ccccc4C5(C)C)cc3)cc21 ; CC1(C)c2ccccc2-c2ccc(N(c3ccc(-c4ccccc4)cc3)c3ccc(-c4ccc5c6c4Oc4ccccc4N6c4ccccc4C5(C)C)cc3)cc21 ; CC1(C)c2ccccc2N2c3ccccc3Oc3c(-c4ccc(-c5ccc(N(c6ccccc6)c6ccccc6)cc5)cc4)ccc1c32 ; CC1(C)c2ccccc2N2c3ccccc3Oc3c(-c4ccc(-c5ccc(N(c6ccccc6)c6ccc(-c7ccccc7)cc6)cc5)cc4)ccc1c32]

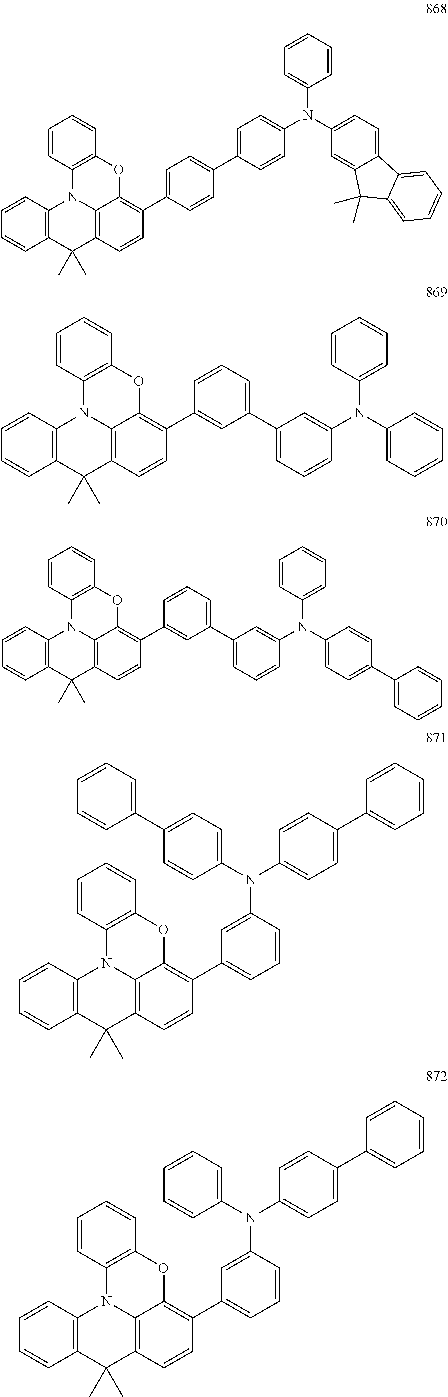

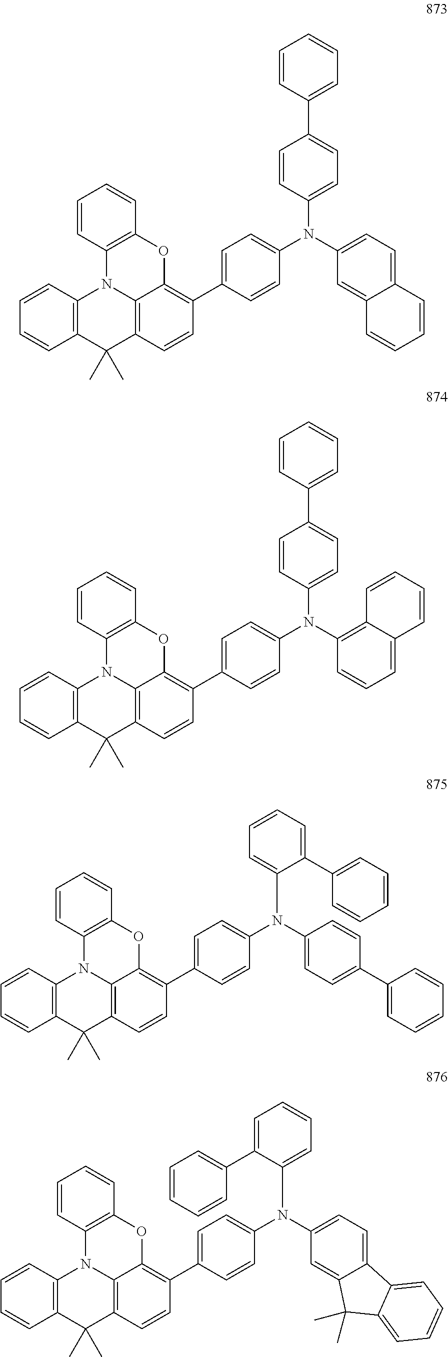 [SMILES: CC1(C)c2ccccc2N2c3ccccc3Oc3c(-c4ccc(N(c5ccc(-c6ccccc6)cc5)c5ccc6ccccc6c5)cc4)ccc1c32 ; CC1(C)c2ccccc2N2c3ccccc3Oc3c(-c4ccc(N(c5ccc(-c6ccccc6)cc5)c5cccc6ccccc56)cc4)ccc1c32 ; CC1(C)c2ccccc2N2c3ccccc3Oc3c(-c4ccc(N(c5ccc(-c6ccccc6)cc5)c5ccccc5-c5ccccc5)cc4)ccc1c32 ; CC1(C)c2ccccc2-c2ccc(N(c3ccc(-c4ccc5c6c4Oc4ccccc4N6c4ccccc4C5(C)C)cc3)c3ccccc3-c3ccccc3)cc21]

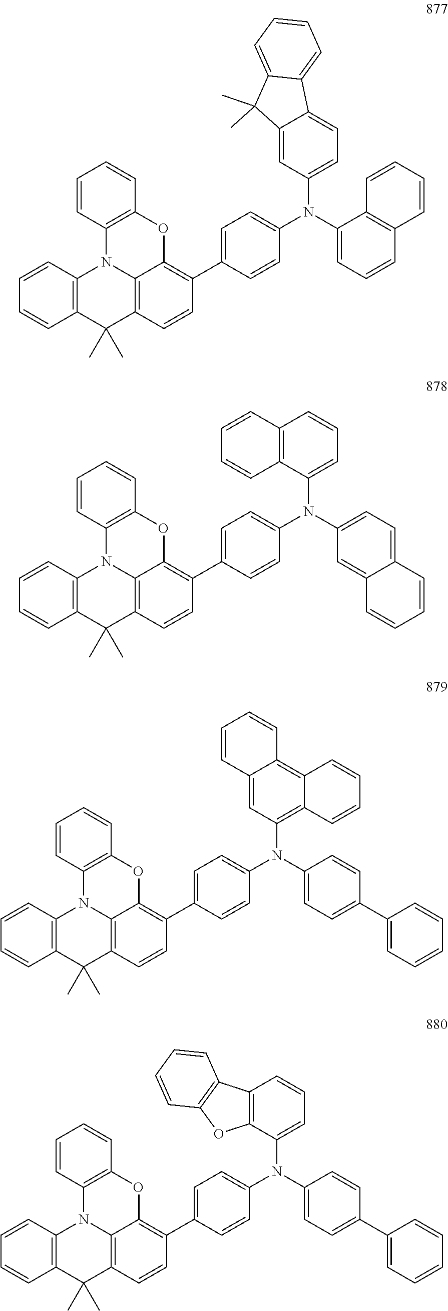 [SMILES: CC1(C)c2ccccc2-c2ccc(N(c3ccc(-c4ccc5c6c4Oc4ccccc4N6c4ccccc4C5(C)C)cc3)c3cccc4ccccc34)cc21 ; CC1(C)c2ccccc2N2c3ccccc3Oc3c(-c4ccc(N(c5ccc6ccccc6c5)c5cccc6ccccc56)cc4)ccc1c32 ; CC1(C)c2ccccc2N2c3ccccc3Oc3c(-c4ccc(N(c5ccc(-c6ccccc6)cc5)c5cc6ccccc6c6ccccc56)cc4)ccc1c32 ; CC1(C)c2ccccc2N2c3ccccc3Oc3c(-c4ccc(N(c5ccc(-c6ccccc6)cc5)c5cccc6c5oc5ccccc56)cc4)ccc1c32]

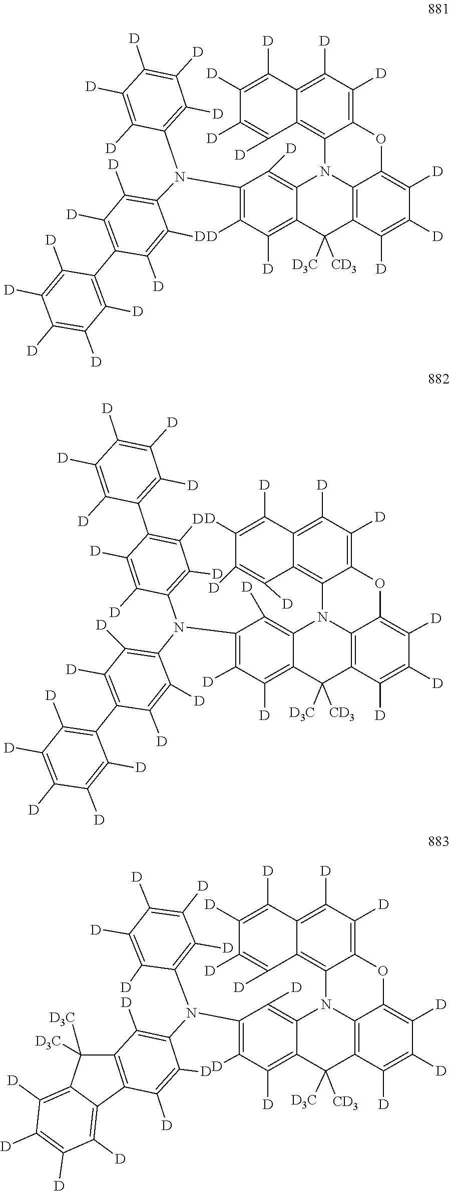

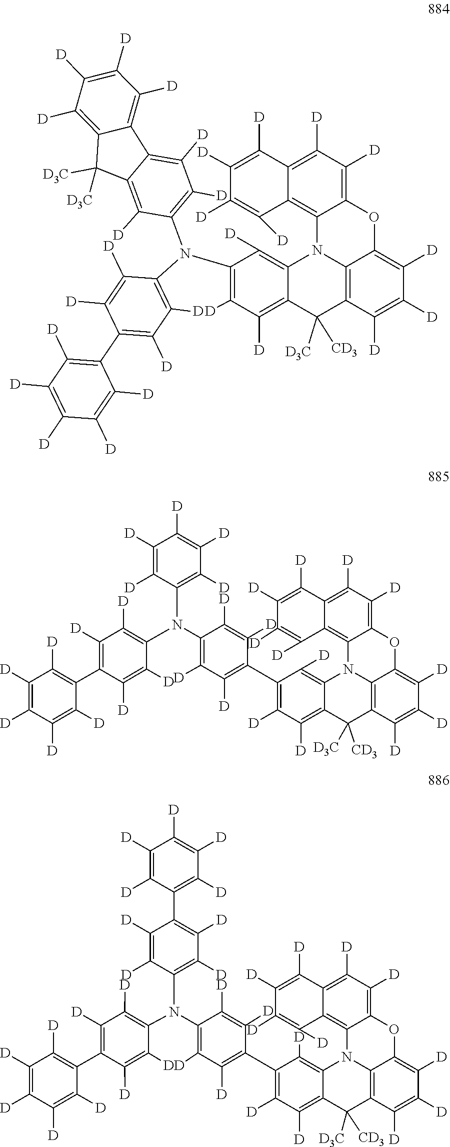

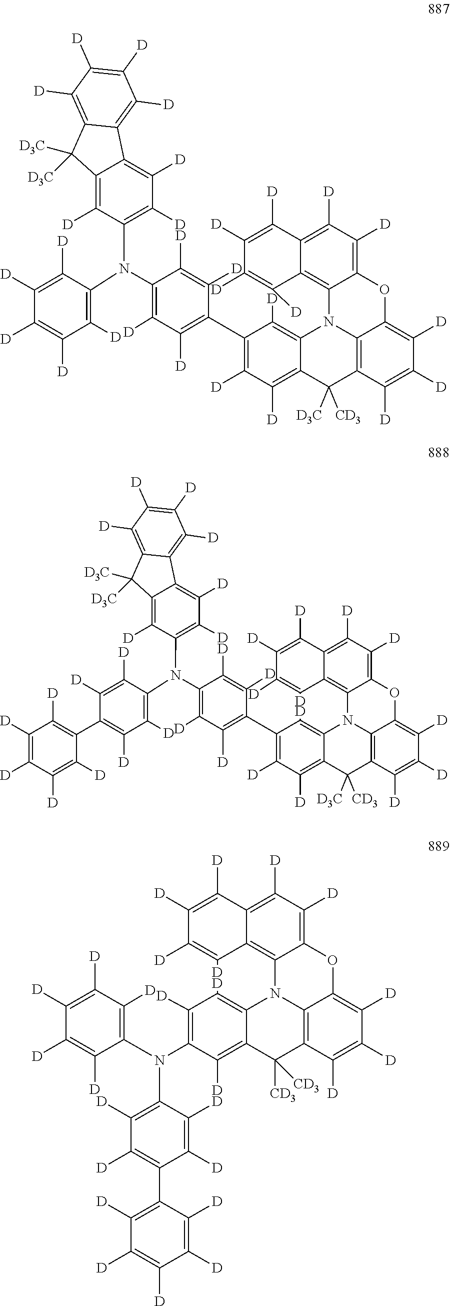

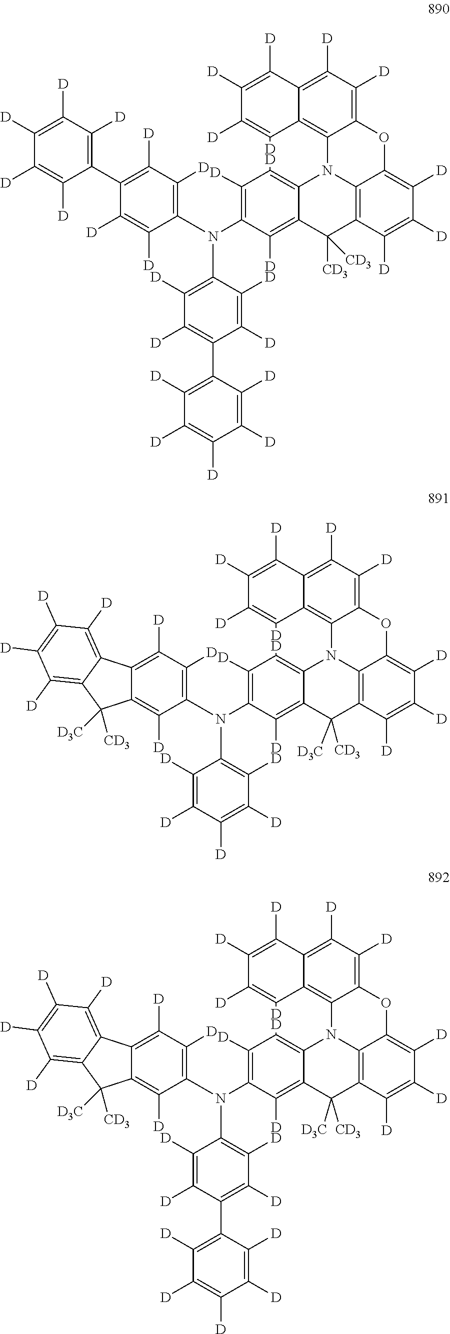

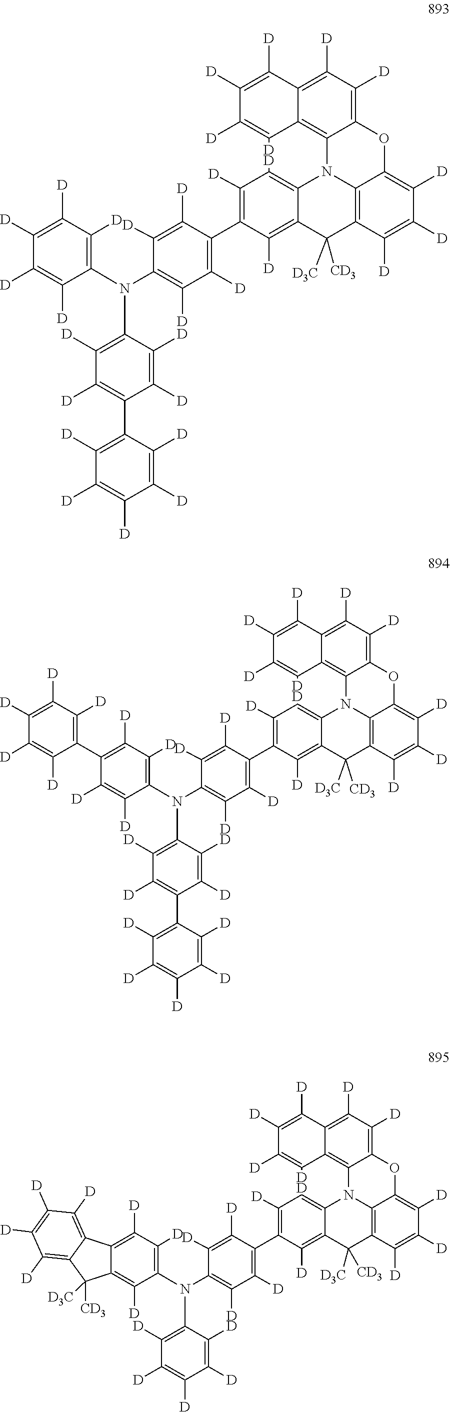

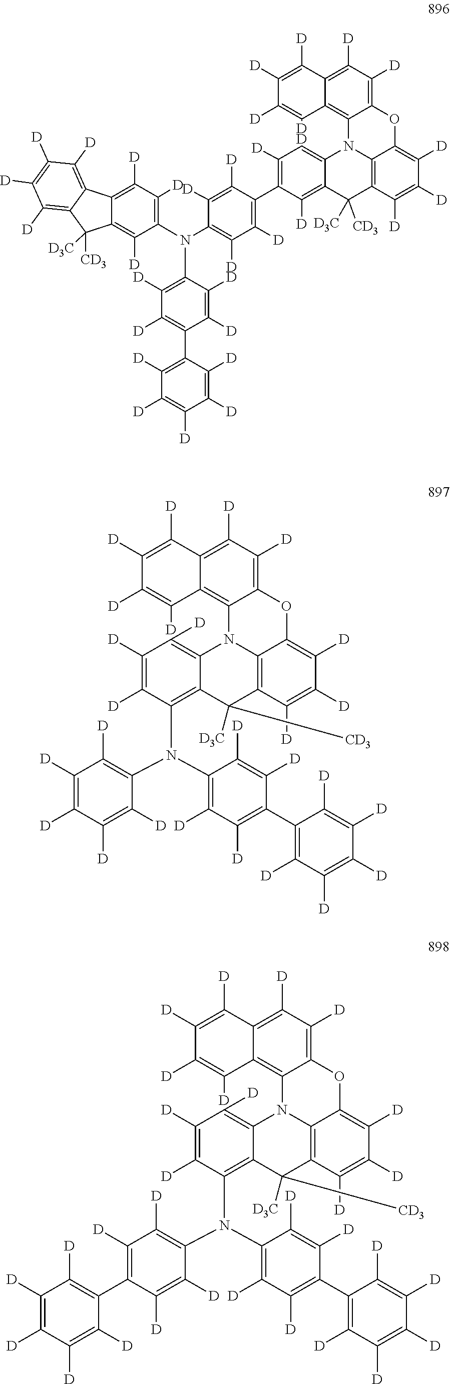

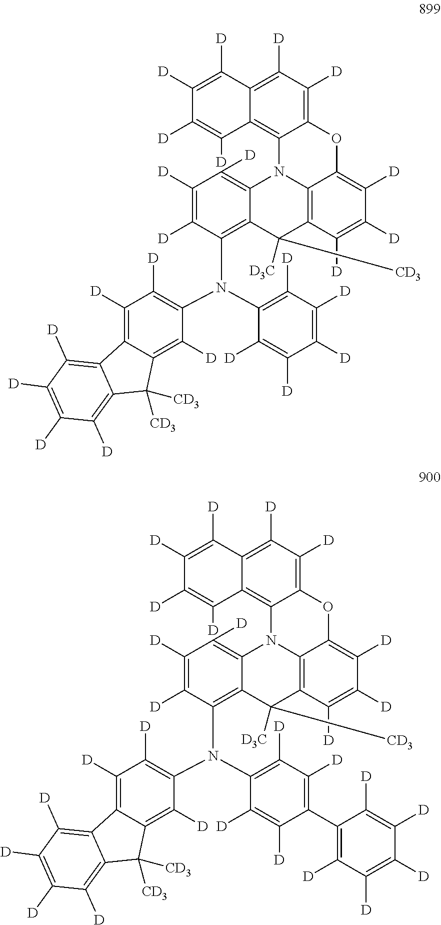

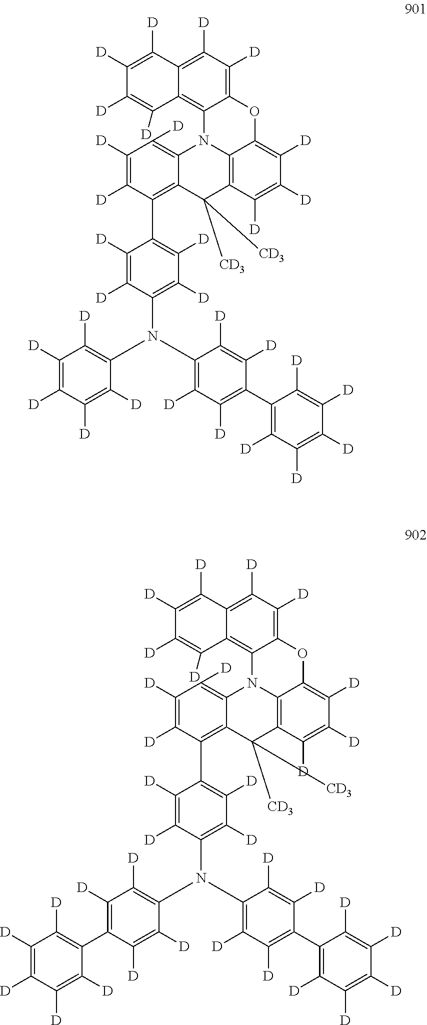

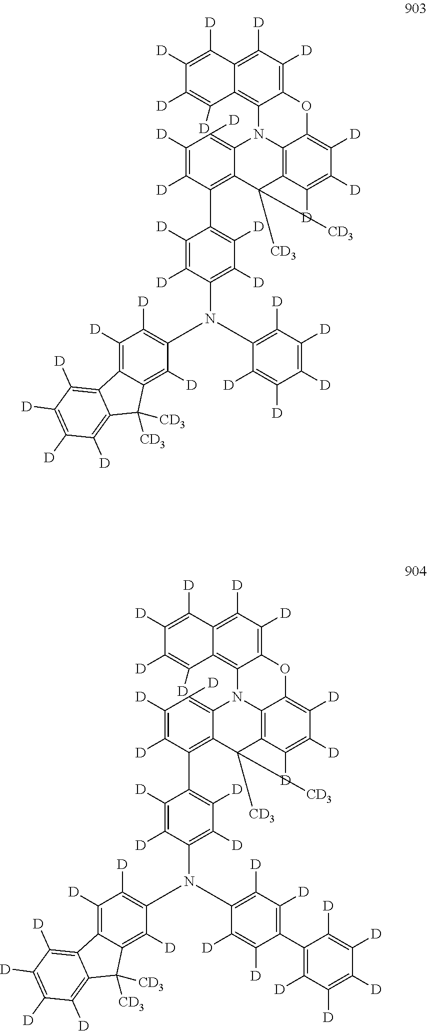

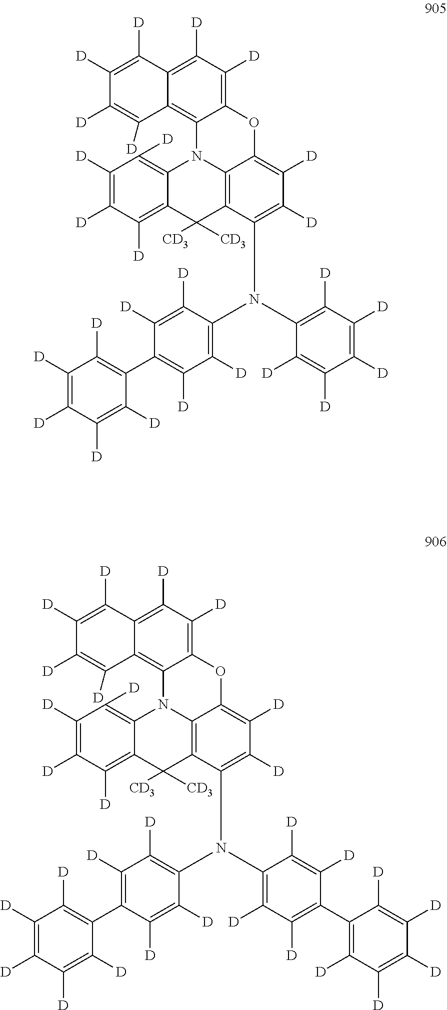

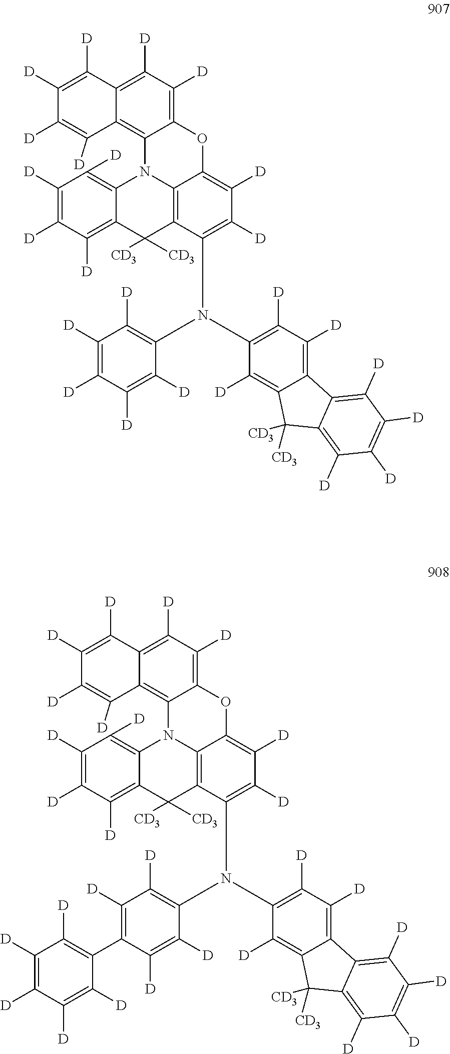

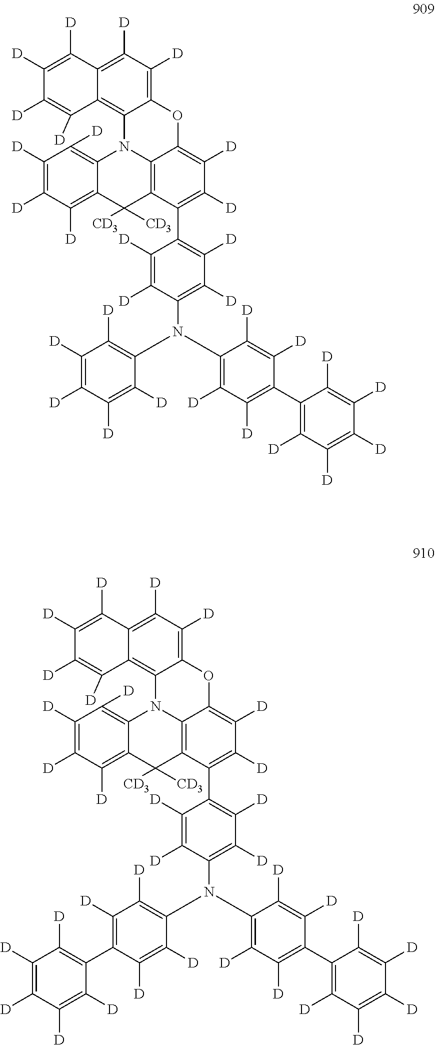

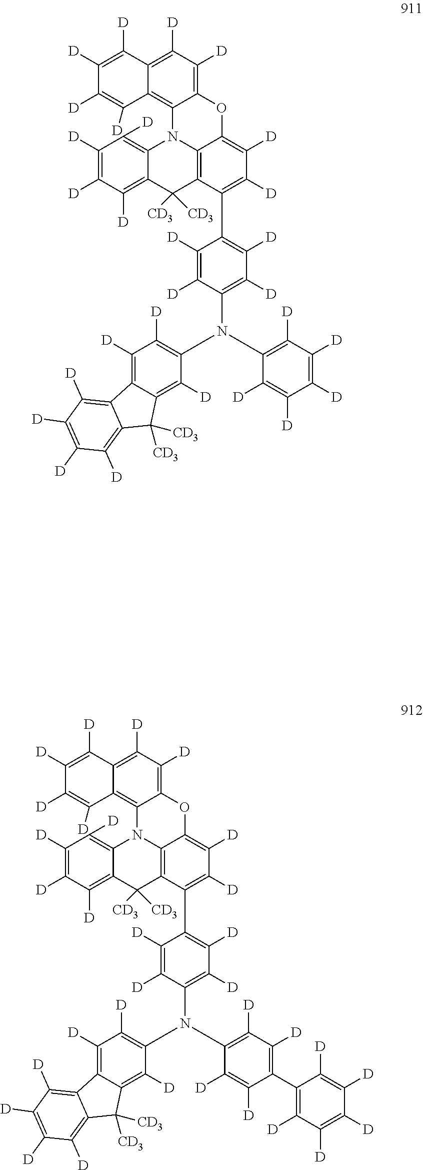

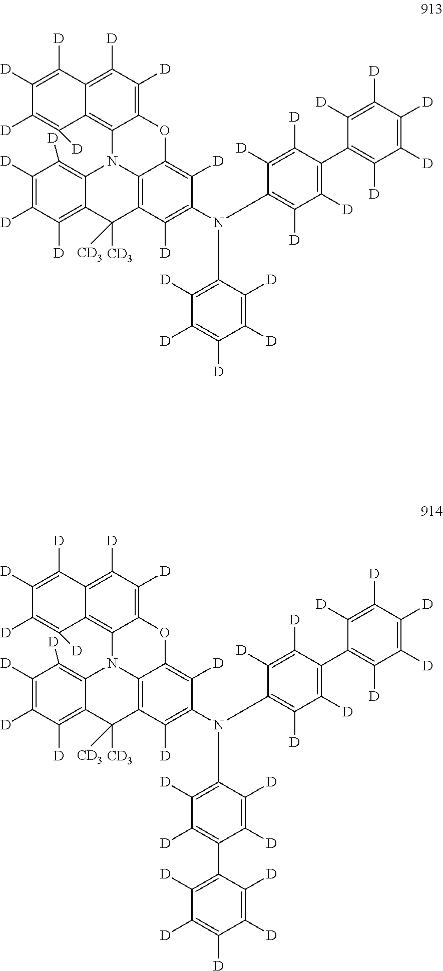

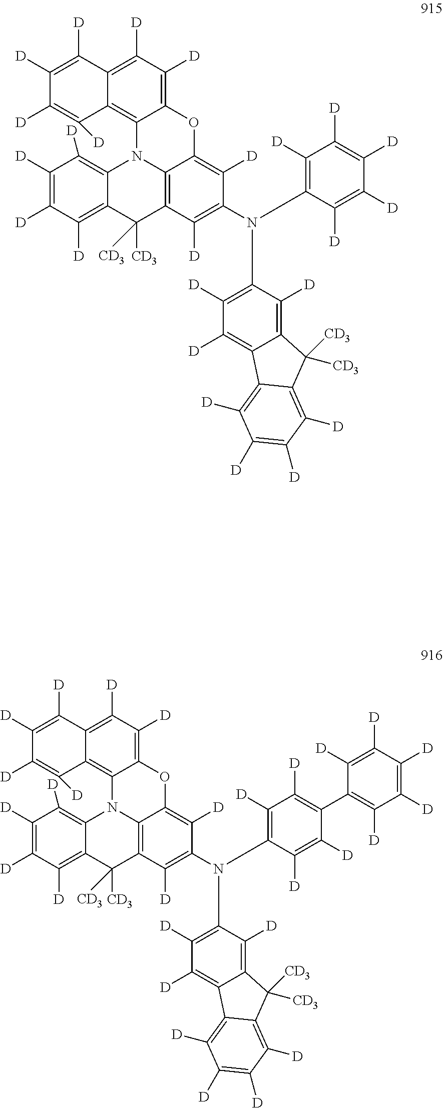

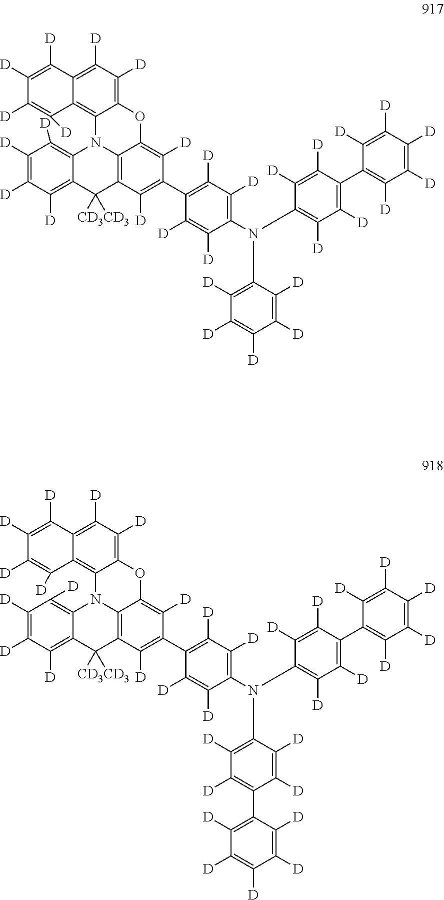

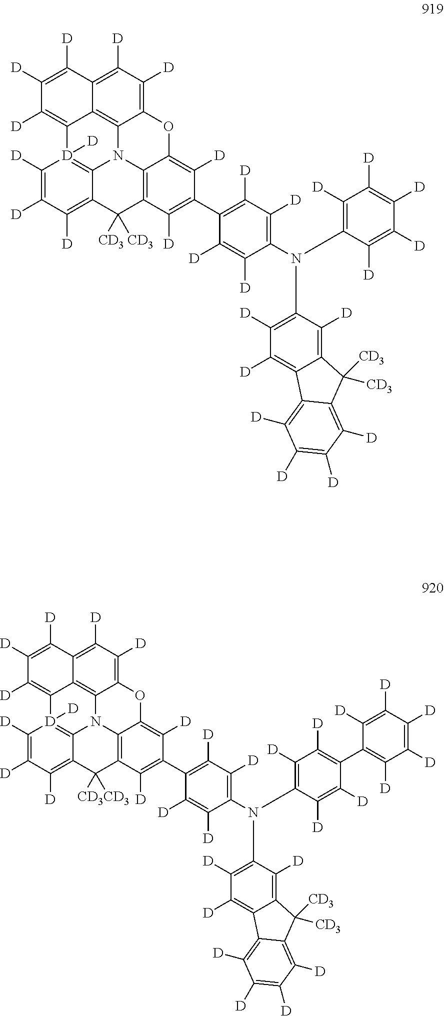

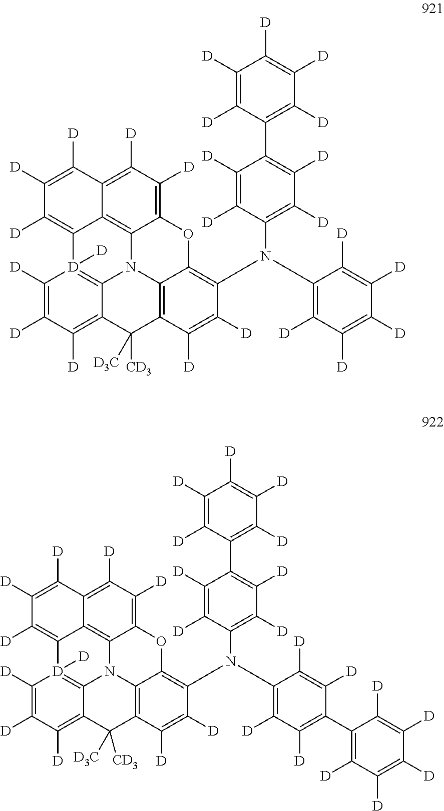

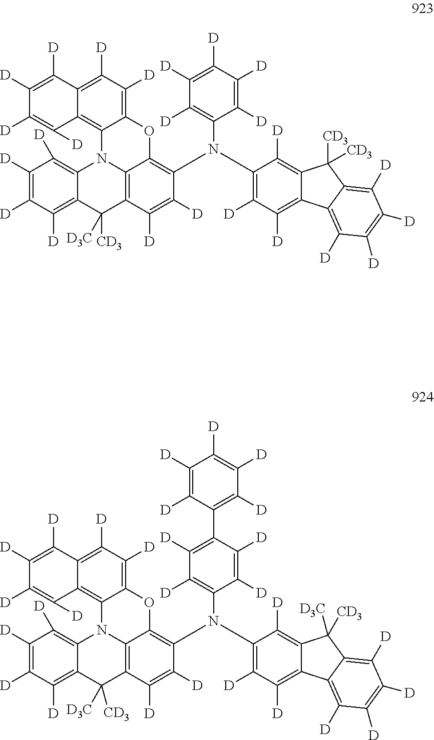

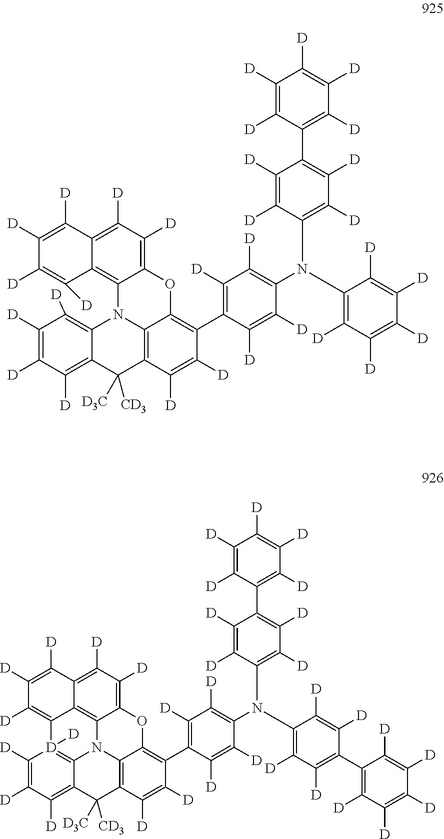

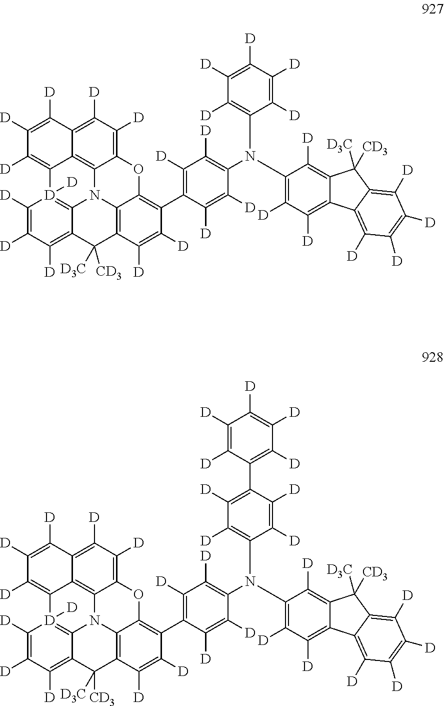

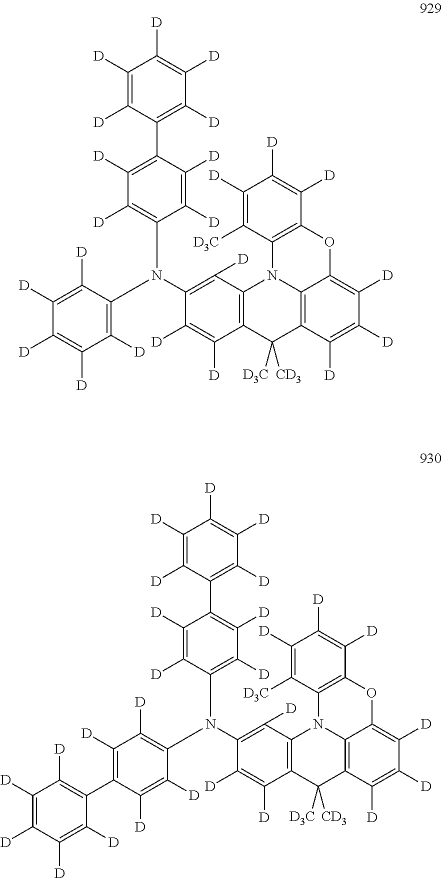

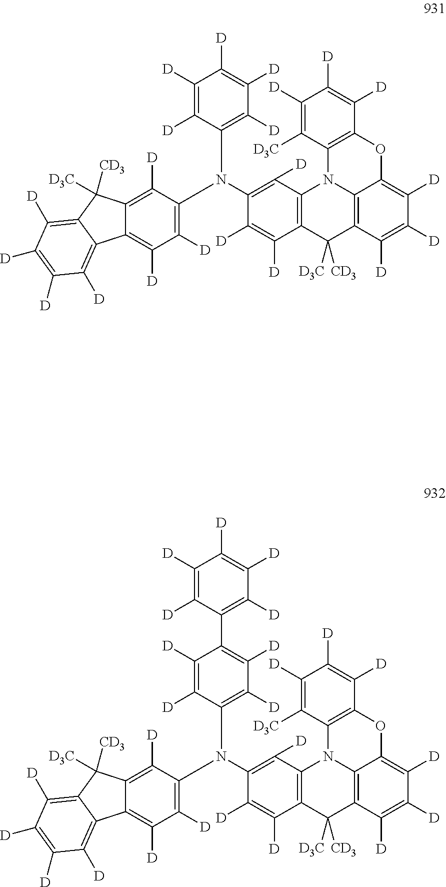

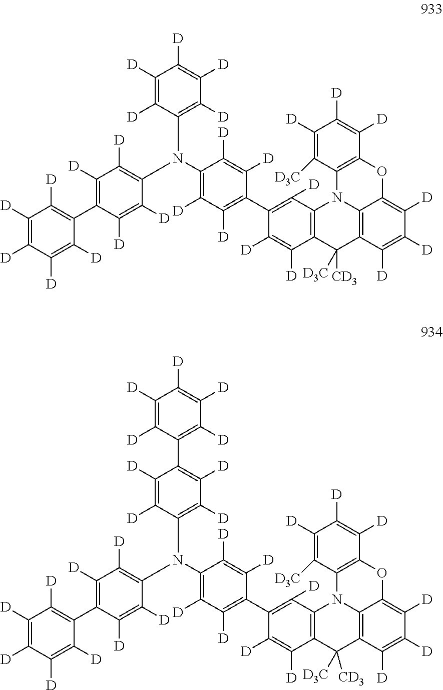

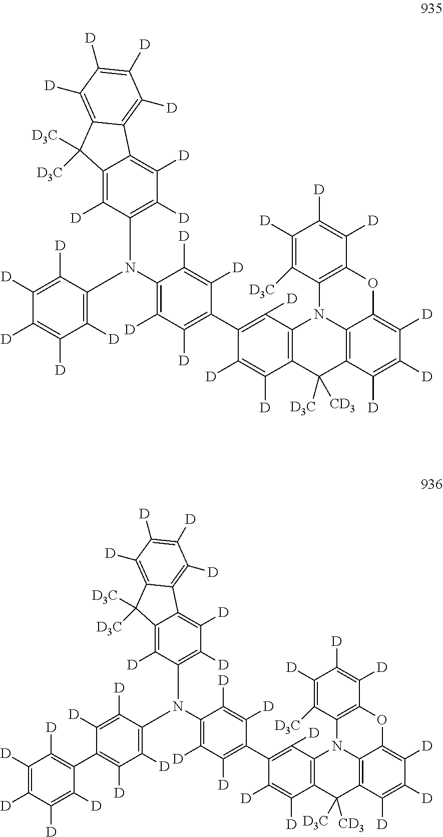

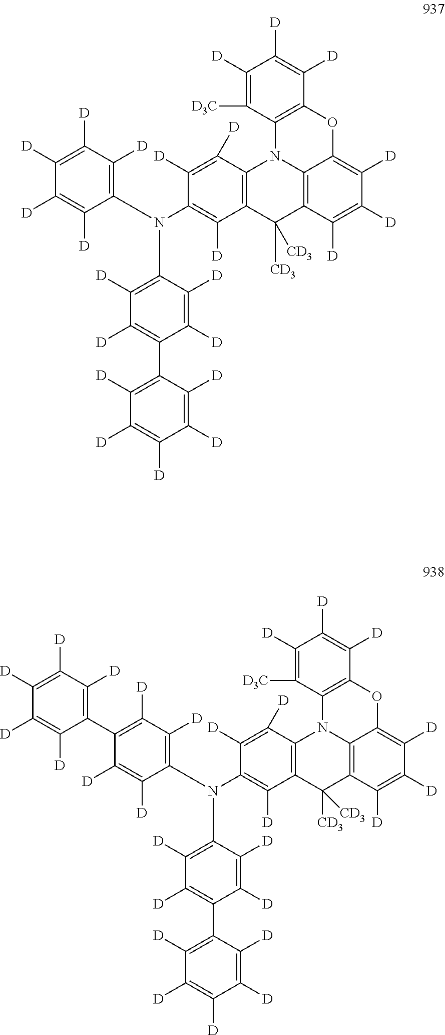

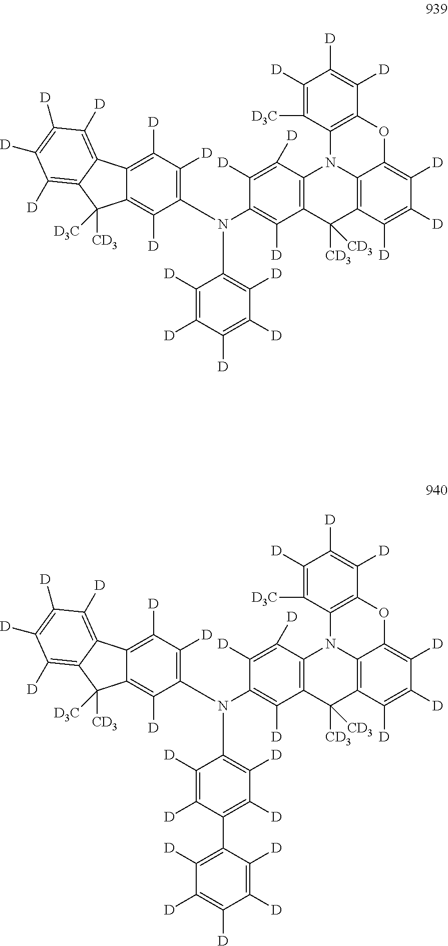

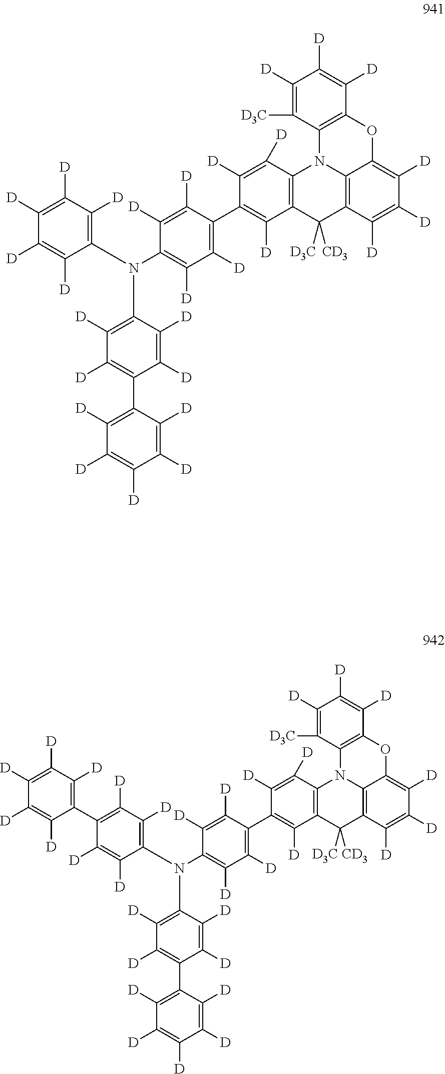

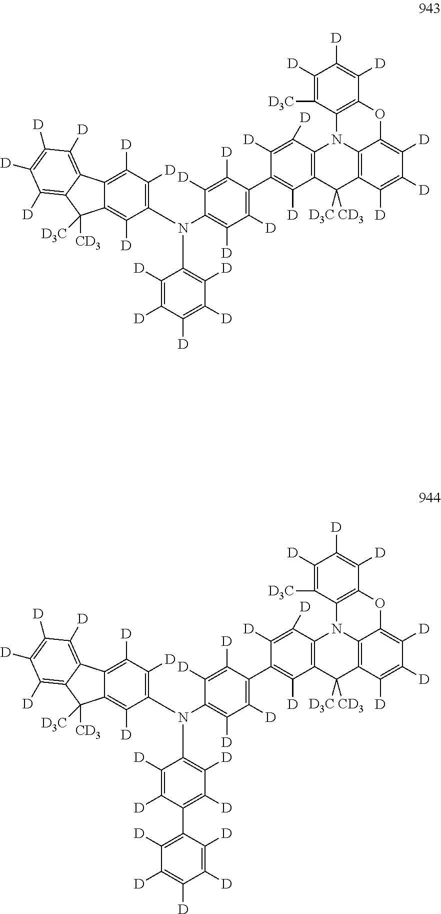

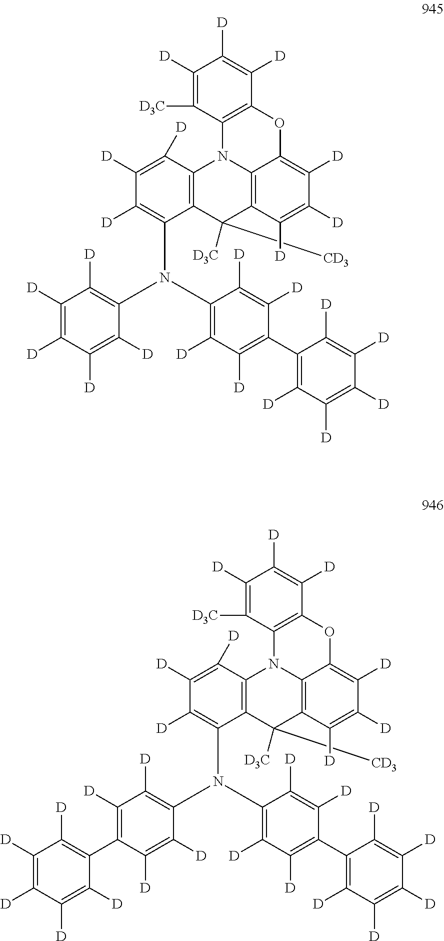

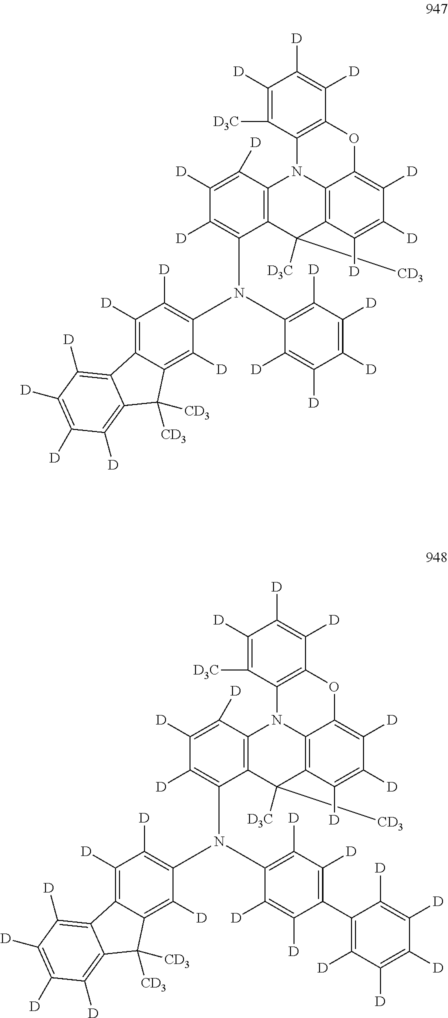

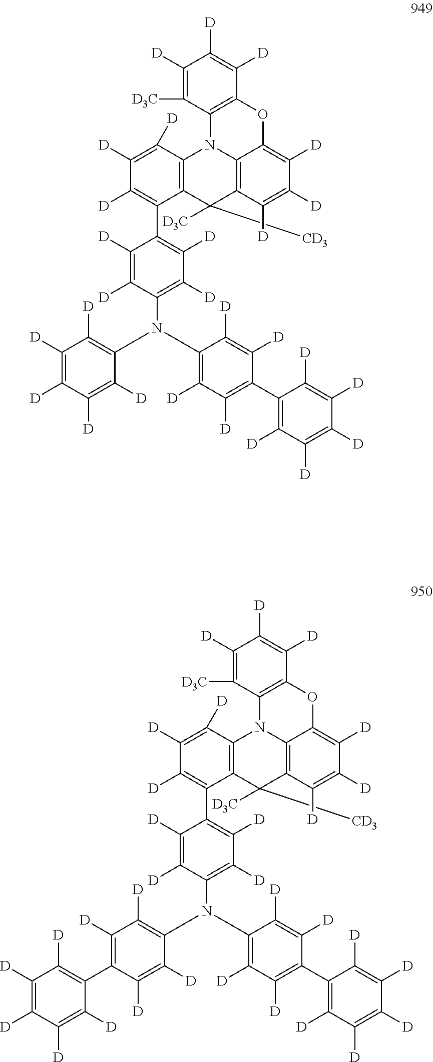

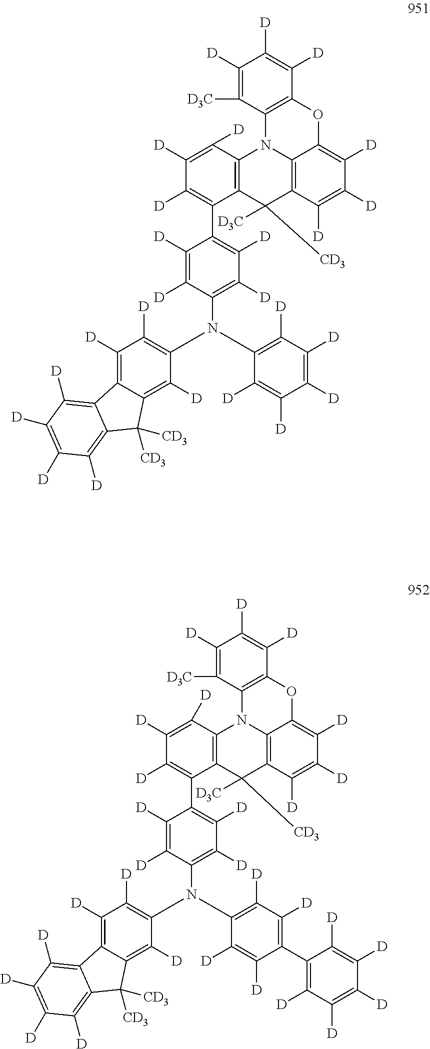

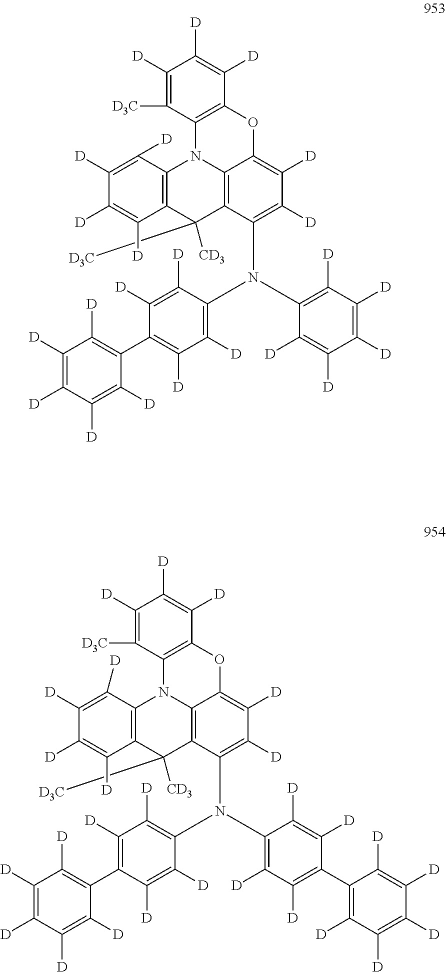

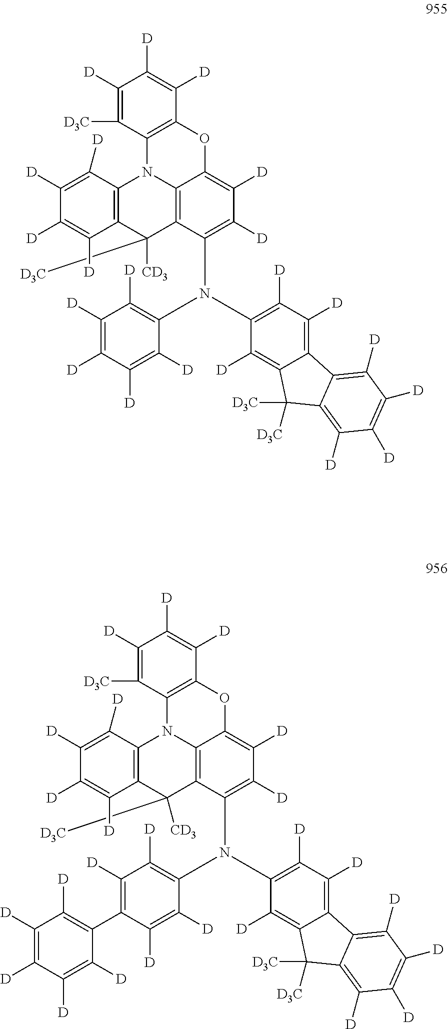

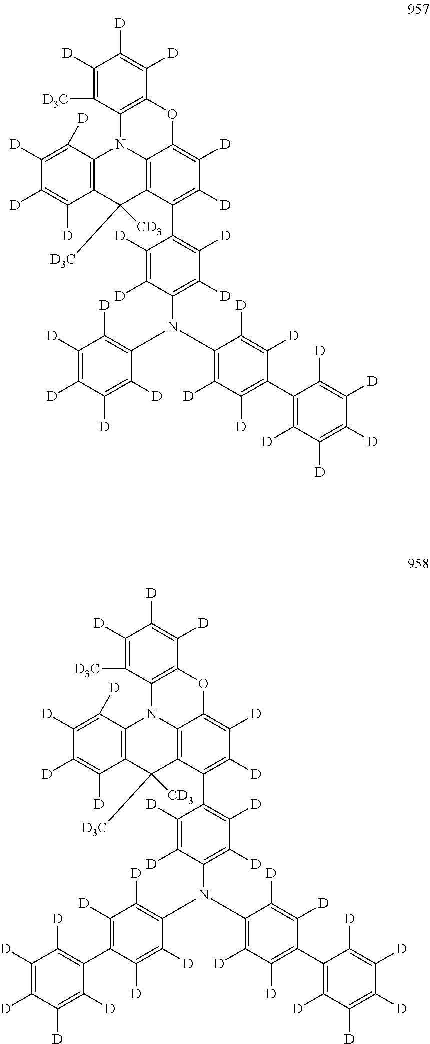

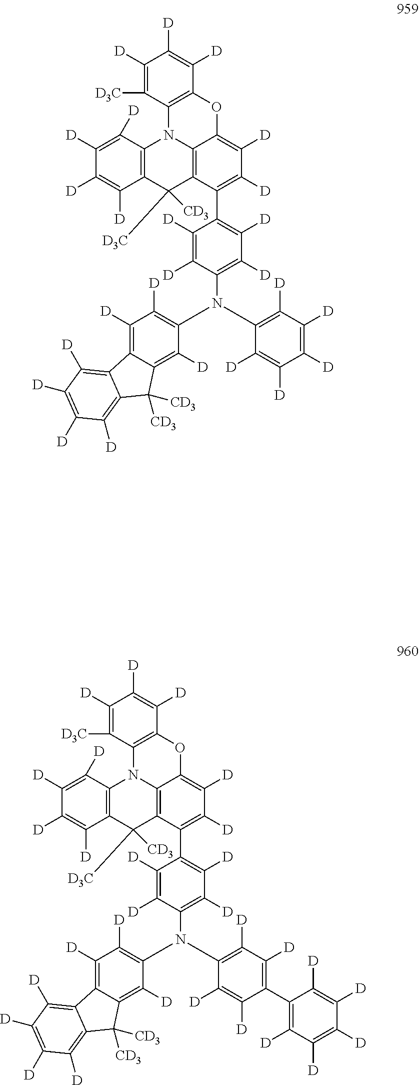

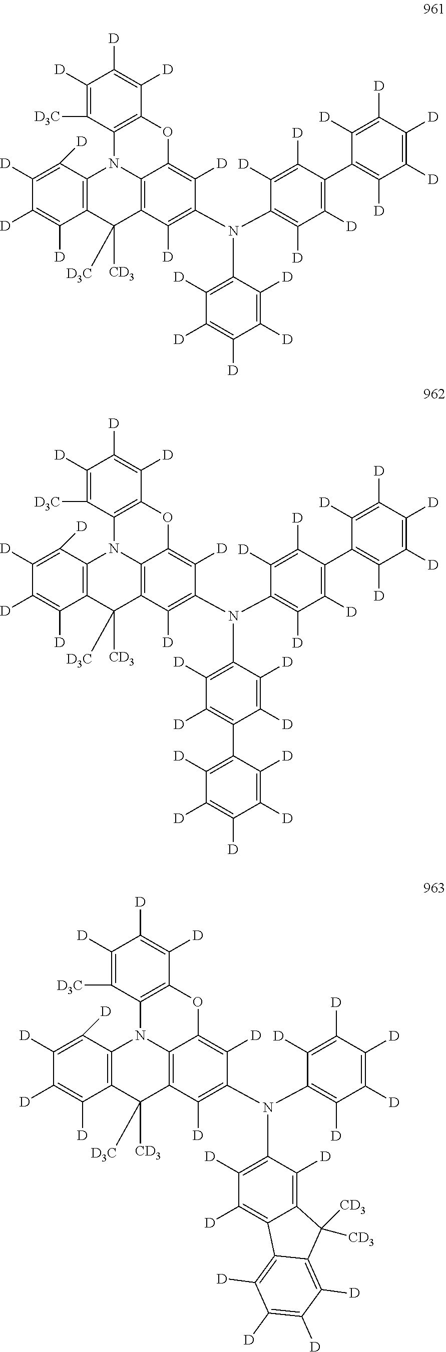

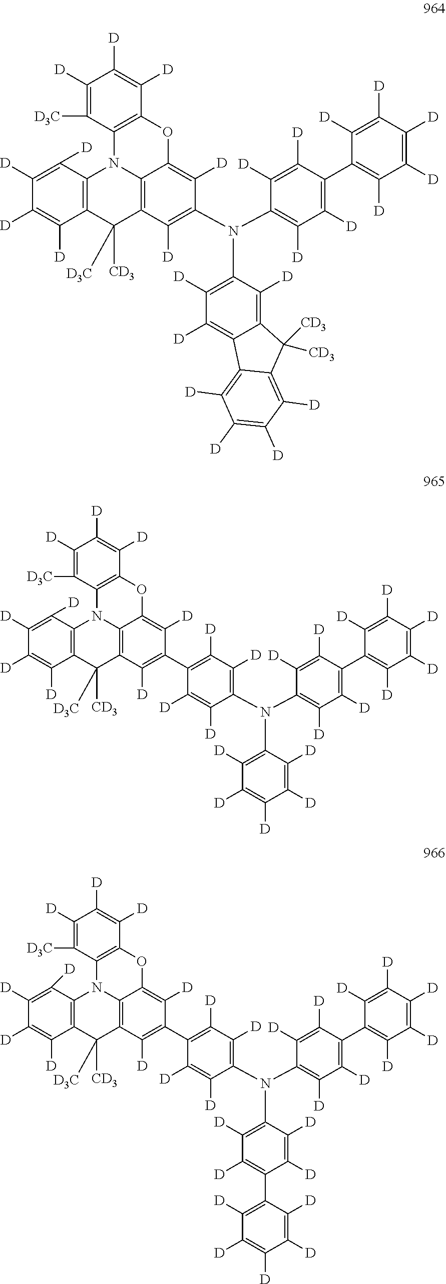

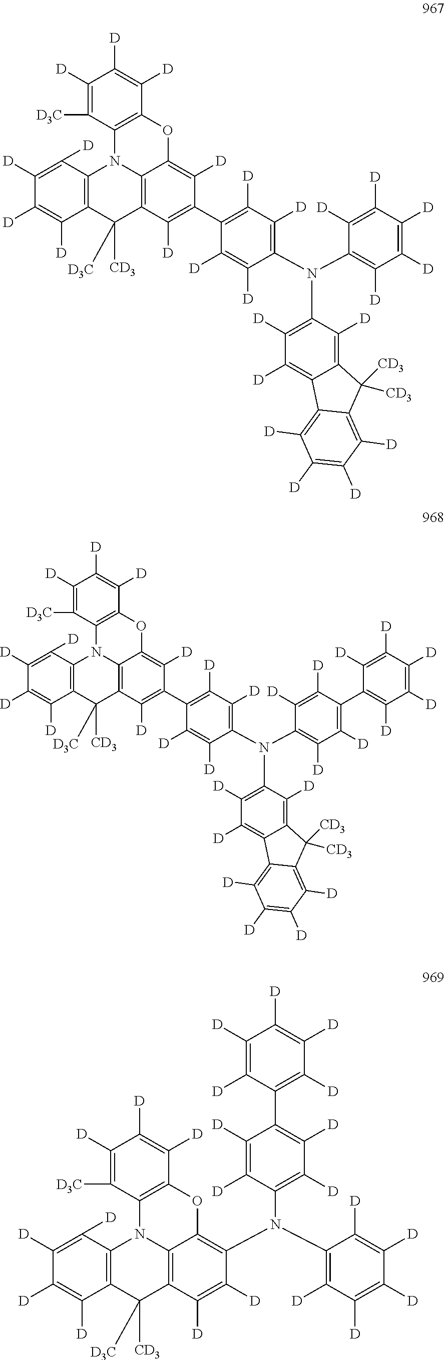

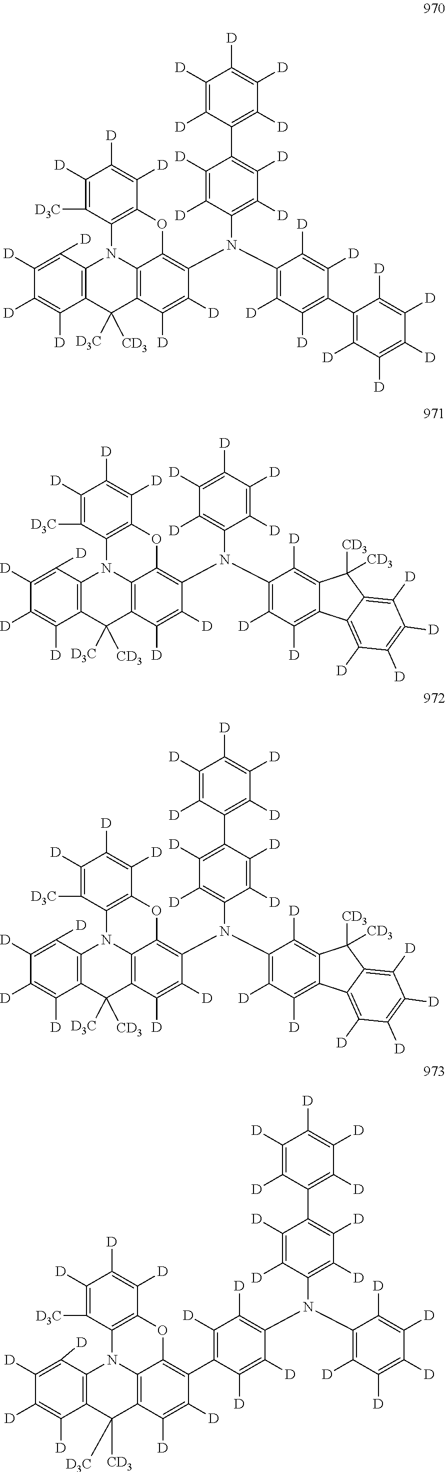

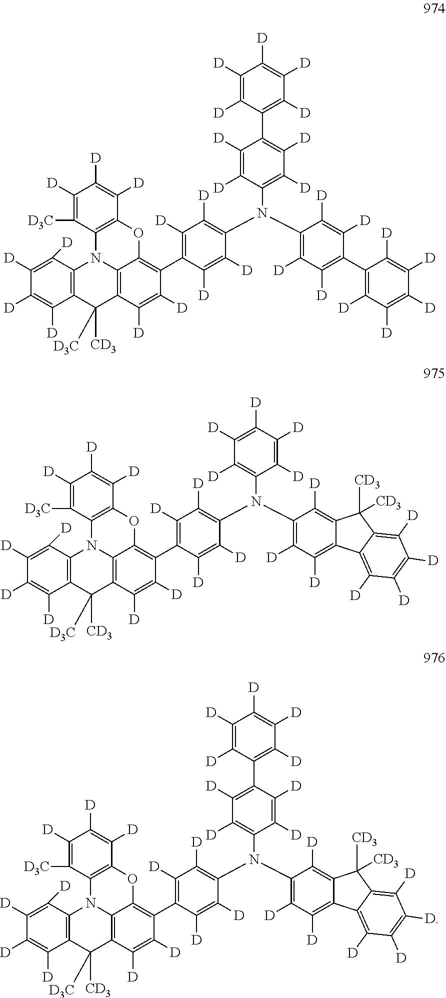

12. An organic light emitting device comprising:
a first electrode;
a second electrode provided to face the first electrode; and
one or more organic material layers provided between the first electrode and the second electrode,
wherein one or more layers of the organic material layers comprise the heterocyclic compound of claim 1.

13. The organic light emitting device of claim 12, further comprising one or more layers selected from the group consisting of a light emitting layer, a hole injection layer, a hole transfer layer, an electron injection layer, an electron transfer layer, an electron blocking layer and a hole blocking layer.

14. A method for manufacturing an organic light emitting device, the method comprising:
preparing a substrate;
forming a first electrode on the substrate;
forming one or more organic material layers on the first electrode; and
forming a second electrode on the organic material layers,
wherein the forming of one or more organic material layers comprises forming the one or more organic material layers using a composition for an organic material layer comprising the heterocyclic compound of claim 1.
